(19) United States
(12) Patent Application Publication
(10) Pub. No.: US 2004/0018957 A1
(43) Pub. Date: Jan. 29, 2004

(54) Crystal structure of a Ras-P13K complex

(76) Inventors: John Eccleston, London (GB); Michael Pacold, Boston, MA (US); Len Stephens, Cambridge (GB); Roger Williams, Cambridge (GB)

Correspondence Address: PALMER & DODGE, LLP, KATHLEEN M. WILLIAMS, 111 HUNTINGTON AVENUE, BOSTON, MA, 02199, US

(21) Appl. No.: 10/371,617

(22) Filed: Feb. 21, 2003

Related U.S. Application Data
(63) Continuation of application No. PCT/GB01/03810, filed on Aug. 24, 2001.

(30) Foreign Application Priority Data
Aug. 24, 2000 (GB) 0020958.5

(51) Int. Cl. A61K 31/00; G06F 19/00; G01N 33/48; G01N 33/50; C12N 9/12; C12N 9/10

(52) U.S. Cl. 514/1; 435/194; 702/19; 435/193

(57) ABSTRACT

The invention relates to a crystal comprising P13K and Ras, and to the use of such crystals in modelling the Ras-P13K interaction and in the design and/or screening of ligands capable of modulating this interaction

### RELATED APPLICATIONS

This application claims priority under 35 U.S.C. §120 to International Application No. PCT/GB01/03810, filed Aug. 24, 2001, which, in turn claims priority to GB0020958.5, filed Aug. 24, 2000.

### BACKGROUND

The present invention relates to a crystal structure for phosphoinositide (PI) 3-kinase (PI3K) and/or Ras. In particular, the present invention relates to a crystal structure for an PI3K:Ras complex and the use of the crystal structure to identify ligands capable of binding to PI3K and/or Ras, and/or capable of modulating the interaction between PI3K and Ras.

The phosphoinositide 3-kinases (PI3Ks) are a family of ubiquitous multidomain signalling proteins which phosphorylate the 3-hydroxyl of phosphoinositides. Mammalian PI3Ks are divided into three classes based on their structure and substrate specificity (Domin and Waterfield, 1997). Class I enzymes are divided into two subclasses by the nature of a tightly associated regulatory subunit. The Class IA PI3K enzymes (p110, α, β, and δ isozymes) interact with an adaptor/regulatory subunit essential for their activation by receptor or non-receptor tyrosine kinases (reviewed in (Vanhaesebroeck and Waterfield, 1999). The sole class IB enzyme, PI3Kγ, binds a p101 subunit that is necessary for heterotrimeric G protein stimulation of phosphatidylinositol (3,4,5)-trisphosphate (PtdIns(3,4,5)P₃) production (Stoyanov et al., 1995; Stephens et al., 1997).

The products of PI3Ks, phosphatidylinositol 3-phosphate (PtdIns(3)P), phosphatidylinositol 3,4-bisphosphate (PtdIns(3,4)P₂) and PtdIns(3,4,5)P₃ affect a variety of targets (Toker and Cantley, 1997). In many cases, the 3-phosphorylated lipids act as tethers to recruit downstream effectors to the membrane. Cellular processes in which PI3Ks play an essential role include suppression of apoptosis (Franke et al., 1997), reorganisation of the actin cytoskeleton (Rodriguez-Viciana et al., 1997), cardiac myocyte growth (Shioi et al., 2000), glycogen synthase stimulation by insulin (reviewed in (Shepherd et al., 1998), TNFα-mediated neutrophil priming and superoxide generation (Condliffe et al., 1998), and leukocyte migration and adhesion to endothelial cells (reviewed in (Wymann et al., 2000).

Because of the significant physiological consequences of PI3K activation, these enzymes are under tight regulatory control, which affects both their substrate specificity and activity. For class I PI3Ks this regulation is achieved by direct interaction of the p110 catalytic subunit with a small number of regulatory proteins. The α, β, and δ PI3Ks associate with an SH2 domain-containing subunit (p85α and splice variants of it, p85β or p55γ) that is indispensable for activation by phosphorylated tyrosine kinases (Carpenter et al., 1993). PI3Kγ is regulated by association with Gβγ subunits of heterotrimeric G proteins. Although Gβγ can bind directly to the p110γ catalytic subunit, a p101 regulatory subunit that tightly associates with p110γ is critical for Gβγ-activated PtdIns(3,4,5)P₃ production (Stephens et al., 1997; Krugmann et al., 1999; Maier et al., 1999). PI3Kβ can also be activated by interaction with Gβγ subunits, and this activation is synergistic with activation by phosphotyrosine peptides (Kurosu et al., 1997; Maier et al., 1999; Murga et al., 2000). Another class of potent activators of PI3Ks is the Ras family of small G proteins. All class I PI3Ks bind Ras in a GTP-dependent manner (Rodriguez-Viciana et al., 1994; Rubio et al., 1997; Vanhaesebroeck et al., 1997; Deora et al., 1998).

The Ras family of membrane localised GTPases transduce a variety of signals in eukaryotic cells (Lowy and Willumsen, 1993; Shields et al., 2000). The exchange of GDP for GTP causes two regions of Ras, known as switch I (residues 32-40) and switch II (residues 60-76), to change conformation (Pai et al., 1989; Milburn et al., 1990). This GTP-induced conformational change allows Ras to bind and activate multiple downstream effectors, which can cooperate to produce the diverse phenotypes characteristic of Ras transformation (White et al., 1995; Marshall, 1996) or function independently in activating survival pathways (Xue et al., 2000). Most of the Ras-dependent signalling is mediated by three downstream effectors: the protein kinase Raf, the exchange factor RalGDS, and PI3K (reviewed in (Wittinghofer and Herrmann, 1995; Feig et al., 1996; Rodriguez-Viciana et al., 1996a).

Although all class I PI3Ks bind Ras, only PI3Kα has been shown to be directly stimulated by Ras.GTP interaction (Rodriguez-Viciana et al., 1994; Rodriguez-Viciana et al., 1996b). Ras-induced activation is synergistic with p85-mediated stimulation of PI3Kα activity by phosphotyrosine peptides. Both stimuli are probably required to achieve optimum PI3K activation in response to extracellular signals. Direct Ras activation of PI3Kα stimulates actin rearrangement and inhibits programmed cell death upon detachment from the extracellular matrix (anoikis) (Khwaja et al., 1997; Rodriguez-Viciana et al., 1997). Ras mutants that activated Raf and RalGDS but not PI3K were incapable of blocking anoikis or stimulating cytoskeletal rearrangement. Other consequences of Ras activation of PI3Ks include cell transformation (Gire et al., 2000), T cell adhesion and migration (Tanaka et al., 1999) and blocking of apoptosis induced by c-Myc (Kauffmann-Zeh et al., 1997) or by NGF deprivation (Xue et al., 2000).

Accordingly, control of Ras activation of PI3K has implications in the control of leukaemias, metastatic cancer and other tumours. The use of small molecules for the inhibition of oncogenic Ras-Raf association has been discussed previously (Barnard et al., (1998) BBRC 247:176-180) and the use of short peptides derived from the H-Ras effector region for this purpose demonstrated. However, the inhibition of Ras-PI3K interaction has not been investigated previously.

Understanding the mechanism of Ras activation has generally been limited by the use of isolated domains to structurally characterise Ras-effector interactions. A recent crystal structure of PI3Kγ (Walker et al., 1999) showed that the Ras binding domain (RBD) of the kinase packs against the catalytic domain. However, no cocrystallisation of PI3K and Ras has been available in the art.

### SUMMARY OF THE INVENTION

We have shown that Ras forms a transient complex with PI3Kγ and activates it in vitro and in vivo. To characterise the PI3K interaction with Ras, we have trapped a Ras.PI3Kγ complex and determined its structure by X-ray crystallography. This structure of Ras in complex with PI3Kγ shows interactions that are unique to PI3Ks, and is a good model for the interaction of PI3Kα with Ras. The conformational changes that take place in PI3Kγ upon Ras binding suggest that an allosteric mechanism, in addition to membrane recruitment, may be important in the Ras-mediated activation of this class of effectors. Ras activation of PI3Kγ is synergistic with Gβγ stimulation. The activation of PI3Kγ by both Ras and by Gβγ is probably necessary for the pivotal role of PI3Kγ in fMLP-dependent neutrophil activation.

The present invention thus provides a crystal comprising PI3K and Ras.

In a further aspect, the present invention provides a crystal structure for a complex comprising PI3K and Ras. The structure of this crystal has been determined and is set forth in Table 2.

Optionally, the crystal according to the invention may comprise one or more molecules which bind to Ras and/or PI3K, or otherwise cocrystallise with the Ras/PI3K complex. Such molecules include ligands, which may be candidate pharmaceutical agents intended to modulate the interaction between Ras and PI3K, or other factors, such as P101, which may be involved in the interaction between Ras and PI3K.

“Ras”, as referred to herein, may be any Ras polypeptide molecule as defined in the art. Particularly preferred is human p21 Ras. The p21 protein is the product of the proto-oncogene ras. Three isozymes of Ras, Ha-Ras (or H-Ras), N-Ras and Ki-Ras are subject to mutation to produce oncogenic variants. The polypeptide used in the crystal according to the invention may be a wild-type polypeptide or a mutant (oncogenic) polypeptide. The primary structure of Ras proteins is well known in the art and available from a number of sources, including GenBank.

Crystals according to the invention may be prepared using full-length Ras polypeptides; preferably, however, the effector domain is employed in isolation. Typically, the effector domain comprises residues 1-166 of human H-Ras, or the equivalent thereof.

“PI3K” may be any PI3K isozyme, of class 1A or 1B. Preferred is PI3Kα or γ. As with Ras, the primary structure of PI3K is known in the art and may be determined from a variety of sources, including GenBank.

The crystal may comprise the full-length PI3 K, but use of the catalytic subunit of PI3K in isolation, preferably a truncated catalytic subunit of PI13K, is preferred. The catalytic subunit or the truncated catalytic subunit typically includes residues 144 to 1102.

Crystals may be constructed with wild-type PI3K polypeptide sequences. However, the transient nature of Ras-PI3K binding makes crystallisation of such complexes more difficult. Accordingly, a PI3K mutant which stabilises the Ras/PI3K complex is advantageously employed in order to stabilise the Ras-PI3K interaction and facilitate crystallisation. Advantageously, the mutation is a V223K mutation.

In a preferred embodiment, the invention provides a crystal having the atomic coordinates set forth in Table 2. It will be understood by those skilled in the art that atomic coordinates may be varied, without affecting significantly the accuracy of models derived therefrom; thus, although the invention provides a very precise definition of a preferred atomic structure, it will be understood that minor variations are envisaged and the claims are intended to encompass such variations. Preferred are variants in which the r.m.s. deviation of the x, y and z co-ordinates for all atoms other than hydrogen is less than 2.5 Å (preferably less than 2 Å, advantageously less than 1 Å, and more preferably less than 0.5 Å or less than 0.1 Å) compared with the coordinates given in Table 2.

The crystals provided in accordance with the present invention may be used to develop models useful for drug design and in silico screening of candidate molecules.

Models and/or atomic coordinates are advantageously stored on computer-readable media, such as magnetic or optical media and random-access or read-only memory, including tapes, diskettes, hard disks, CD-ROMs and DVDs, flash memory cards or chips, severs and the internet. The computer capable of reading the medium according to the invention may be any suitable computer, such as a Windows®, Macintosh®, LINUX® or UNIX®-based system. The invention accordingly provides a computer-readable medium having stored thereon a model of PI3K/Ras interaction.

Models of PI3K/Ras interaction may be used to design or screen for drugs capable of modulating this interaction. Such drugs may be capable of or designed to alleviate the resistance to apoptosis, cytoskeletal rearrangement and anoilds conferred through PI3K activation by Ras. Methods for screening and designing drugs based on a crystal structure-derived model of a target are known in the art and described in more detail below. The invention extends to both operator-controlled modelling and automated modelling techniques based on a PI3K/Ras crystal structure-derived target.

Modelling may be carried out, in particular, on the regions of the target complex which are known to be required for PI3K-Ras interaction. As shown herein, the β-2 strand of Ras and the Rβ2 strand of PI3K are essential in the interaction of these two molecules. Hence, the model according to the invention advantageously comprises the β-2 strand of Ras and/or the Rβ2 strand of PI3K.

PI3K is shown herein to effect a unique interaction, amongst polypeptides activated by Ras, with both the switch I and the switch II region of Ras. Accordingly, modelling is advantageously carried out to include the interaction of PI3K with both switch I and switch II regions.

Furthermore, the 225-267 loop of PI3K is demonstrated to be essential for Ras-PI3K interaction. In a preferred aspect, therefore, modelling is carried out to include the PI3K 225-267 loop.

The invention moreover provides ligands capable of influencing Ras/PI3K interaction, which may be designed or selected by the methods according to the invention.

Ligands according to the invention are useful in the treatment of diseases associated with cell proliferation and transformation; the invention accordingly provides the use of a ligand as described above in the manufacture of a medicament to treat and/or prevent disease in a mammal, and a method for treating and/or preventing disease in a mammal comprising administering such ligands.

### BRIEF DESCRIPTION OF DRAWINGS

FIG. 1: (A) In vitro activation of PI3Kγ by H-Ras.GTP. The levels of [³²P]-labelled phosphoinositides were measured and data for [³²P]-PtdIns(3)P, [³²P]-PtdIns(3,4)P₂ and PtdIns(3,4,5)P₃ are shown. The DASAA PI3Kγ mutant contains the T232D, K251A, K254S, K255A, and K256A mutations. The data are representative of three experiments and were normalised by the amount of PI3Kγ used in the assay.

(B) In vivo activation of the p110γ/p101 heterodimer by H-Ras.GTP. COS-7 cells were transfected with expression vectors as indicated. Data were collected as above, and are representative of three experiments. (C) G12V H-Ras activation of the wild type and DASAA mutant p110γ in the absence of p101 (D) Synergistic activation of p110γ/p101 heterodimer with Gβγ and G12V H-Ras.

FIG. 2: (A) Solution binding of PI3Kγ RBD mutants to N-Ras.mant-GMPPNP. All mutants are in the 144-1102 construct. Titration of some of the mutants was limited by reduced mutant solubility. All error bars represent the standard deviation of at least two measurements. K_{d} values are listed in the adjacent table. For mutants with no detectable binding, the K_{d} is listed as >50. (B) Transient kinetic measurements of the association and dissociation rates of the PI3Kγ 144-1102 construct to N-Ras.mant-GMPPNP. The pseudo first order association rates were determined at the PI3Kγ concentrations shown. (C) Transient and steady-state measurements of mant-adenine nucleotide binding to PI3K. All data were collected by fluorescence energy transfer from tryptophan to mant. The K_{d} values were determined by equilibrium titration independently of the kinetic data.

(D) Steady-state binding of PI3Kγ 114-1102 to full length, unprocessed H-Ras mutants. The data were obtained as in figure A. For comparison, PI3Kγ binding to H-Ras G12V loaded with mant-GDP is shown.

FIG. 3: (A) Diagram of interactions between PI3Kγ and Ras. Also shown are the residues that hold the 255-267 loop in place. The RBD is coloured purple, the Ras is coloured orange, and the catalytic domain is yellow. Putative hydrogen bonds are indicated by dashed lines, and possible salt bridges by dotted lines. PI3Kγ residues that can be mutated to eliminate or attenuate binding are coloured red or blue, respectively. Ellipses indicate residues that were mutated to enhance binding. The V223K tighter binding mutant is shown hydrogen bonding to Ras Glu-37. (B) A closer view of the interface between the RBD (purple) and Ras (orange). The switch I and switch II regions of Ras are coloured pale and dark blue, respectively. Residues that were mutated or that form interactions between the two proteins are labelled. Boxes around residue labels denote mutations that abolish binding, and ellipses indicate mutations that enhance binding. The 255-267 loop that becomes ordered on binding is coloured dark green. The GMPPNP and Mg²⁺ in Ras are rendered in gray. (C) Solvent-accessible surface of the Ras.PI3Kγ complex. The Ras (orange) and four domains of the PI3Kγ, comprising the RBD purple), C2 domain (cyan), helical domain (green) and N and C-terminal lobes of the catalytic domain (red and yellow) are shown. The N-terminal linker is rendered in white. (D) Ribbon diagram of the Ras.PI3Kγ complex. The colour scheme is the same as the previous panel. The location of the γ phosphate of ATP/PI3Kγ structure is marked with a large gray sphere. This location roughly corresponds to the phosphoinositide headgroup binding site.

FIG. 4: (A) Stereo view of the PI3Kγ RBD.Ras complex. The molecules are coloured as in FIG. 3. Lines are traced from the centroid of the RBD through the Cα of Thr-232, then to the centroid of Ras bound to Raf (red sphere and dotted line), RalGDS (yellow sphere and dashed line), and PI3Kγ (cyan sphere and solid line). (B) The Rap1A.RafRBD complex (PDB:1gua). (C) The Ras.RalGDS RBD complex (PDB:11fd). The orientation of the Ras in this complex is more similar to that of Ras in the PI3Kγ complex than that of Rap1A in the RafRBD complex.

FIG. 5: (A) Overview of the conformational changes in the Ras.PI3Kγ structure. The structure of the enzyme bound to ATP is traced through the α carbons as a black dotted line. The structure of the Ras.PI3Kγ complex is traced in solid lines and the domains are coloured as in FIG. 3. A gray sphere marks the location of the γ-phosphate of ATP in the structure of the enzyme in the absence of Ras. This would presumably be adjacent to the phospholipid headgroup binding site. (B) A view of the conformational changes in the catalytic domain. The catalytic domains from free PI3K and kinase complexed with Ras were superimposed on the N-terminal lobe of the catalytic domain. The domain from the isolated PI3Kγ is traced as a dashed black line, and the C-α trace of the PI3K catalytic domain is displayed in solid lines.

FIG. 6: A model of the Ras.PI3Kγ complex at a putative membrane surface. All regions not visible in the structure are drawn as dashed lines. Lys-973 marks the substrate-binding loop. The 20-residue C-terminal tail of Ras was arbitrarily modelled to illustrate that this peptide could easily span the gap between the RBD-bound Ras and the putative membrane surface. The location of the farnesyl group is indicated schematically. Potential membrane-interacting residues at the tips of the catalytic domain loops are labelled.

FIG. 7 shows results of experiments showing inhibition of H-Ras G12V signalling by RBD-Ras Tail chimeras. Transfection of the Raf-1 RBD linked to an H-Ras tail reduces the level of oncogenic H-Ras G12V signalling (as measured by a luciferase assay). The Raf-1 RBD linked to a mutant H-Ras tail that casnnot ge lipid modified or to N- or K-Ras tails is incapable of inhibiting the H-Ras G12V signalling to the same extent.

### DETAILED DESCRIPTION OF THE INVENTION

In one aspect of the present invention, there is provided a crystal comprising Ras and PI3K.

As used herein, the term “crystal” means a structure (such as a three dimensional (3D) solid aggregate) in which the plane faces intersect at definite angles and in which there is a regular structure (such as internal structure) of the constituent chemical species. Thus, the term “crystal” can include any one of: a solid physical crystal form such as an experimentally prepared crystal, a 3D model based on the crystal structure, a representation thereof such as a schematic representation thereof or a diagrammatic representation thereof, a data set thereof for a computer.

The mammalian ras gene family consists of the harvey and kirsten ras genes (c-Hras1 and c-Kras2), an inactive pseudogene of each (c-Hras2 and c-Kras1) and the N-ras gene. They differ significantly only in the C-terminal 40 amino acids. These ras genes have GTP/GDP binding and GTPase activity, and their normal function may be as G-like regulatory proteins involved in the normal control of cell growth. Mutations which change amino acid residues 12, 13 or 61 activate the potential of Ras to transform cultured cells and are implicated in a variety of human tumours. The N-ras gene specifies two main transcripts of 2 Kb and 4.3 Kb. The difference between the two transcripts is a simple extension through the termination site of the 2 Kb transcript. The N-ras gene consists of seven exons (-I, I, II, III, IV, V, VI). The smaller 2 Kb transcript contains the VIa exon, and the larger 4.3 Kb. transcript contains the VIb exon which is just a longer form of the VIa exon. Both transcripts encode identical proteins as they differ only the 3′ untranslated region. The sequence of human N-Ras is available on GenBank under accession no. NM_{—}002524, gi 6006027.

c-Ha-ras1 is the normal progenitor of the transforming gene found in several human tumour cell lines (T24 bladder carcinoma; EJ bladder carcinoma; Hs242 lung carcinoma; SK2 melanoma; and HS578T mammary carcinosarcoma). The only difference within the coding exons of the c-Ha-ras1 proto-oncogene and the oncogene of the T24 and EJ cell lines is a ‘g’ to ‘t’ transversion within codon 12 that results in the substitution of valine for glycine at this position in the p21 protein encoded by c-Ha-ras1. The mutation responsible for transforming ability of the p²¹ protein in the SK2 and Hs242 cell lines is an ‘a’ to ‘t’ transversion within codon 61 that results in the substitution of leucine for glutamine at this position. The mutation responsible for transforming ability of the p21 protein in the HS578T cell line is a ‘g’ to ‘a’ transition within codon 12 that results in the substitution of Aspartic acid for glycine at this position. A region of repeated DNA consisting of the 28 bp consensus sequence ‘cactcccccttctctccaggggacgcca’ begins at position 4755. The repeat occurs 29 times in the plasmid used for this sequence but may occur more times in the native DNA. This region is known to be unessential for transforming activity. The sequence of H-Ras is available on GenBank, Accession No. J00277, gi 190890.

The sequence of human K-Ras is available under accession no. M54968, gi 1815608.

The sequence of PI3K is available in GenBank under accession no Y11312, gi 2808446; PI3Kγ is available under Accession no. P48736.

In a preferred embodiment of this aspect of the invention, the crystal comprises the effector domain of Ras and the catalytic subunit of PI3K, isolated form the remainder of the full-length polypeptides. The effector domain of Ras comprises residues 1-166 of human H-Ras, or the equivalent thereof in other ras polypeptides. The catalytic subunit of PI3K comprises residues 144 to 1102. Advantageously, it contains a V223K mutation.

In a highly preferred embodiment of this aspect of the invention, the crystal has the structural coordinates as shown in Table 2.

As used herein, the term “structural co-ordinates” refer to a set of values which define the position of one or more amino acid residues with reference to a system of axes.

The present invention also provides a crystal structure of a PI3K/Ras complex. The structure of the crystal may be solved by any known method, for example by X-ray diffraction followed by multiple anomalous dispersion (MAD).

The crystal structure of a PI3K/Ras complex of the present invention provides information about the overall structure of each constituent thereof and about the particular elements of secondary structure for each constituent which are involved in complex formation. In this respect, the crystal structure indicates that the regions which are important in the interaction include residues 25-41 and 60-74 of Ras and residues 220-267 of PI3K. This includes the switch I and switch II regions of Ras.

As used herein, the term “α-helix” means a helical or spiral configuration of a polypeptide chain in which successive turns of the helix are held together by hydrogen bonds between the amide (peptide) links, the carbonyl group of any given residue being hydrogen-bonded to the imino group of the third residue behind it in the chain. This is the case for all of the carbonyl and amide groups of the peptide bonds of the main chain. Typically, the α-helix has 3.6 residues per turn and the translation or pitch along the helical axis is 1.5 Å per residue and 5.4 Å per turn. The helix may be left- or right-handed, the latter being much more common. The α-helix is one of the two basic elements of the secondary structure adopted by the polypeptide chain within the hydrophobic core of a globular protein. The other basic element is the β strand.

β strands are usually from 5 to 10 residues long and are in an almost fully extended conformation with φ, Ψ angles within the broad structurally allowed region in the upper left quadrant of the Ramachandran plot. These β strands are aligned adjacent to each other such that hydrogen bonds can form between C'O groups of one β strand and NH groups on an adjacent β strand and vice versa. Parallel or antiparallel β sheets may be formed from several such β strands which are “pleated” with C_{α} atoms successively a little above and below the plane of the β sheet. The side chains follow this pattern such that within a β strand they also point alternatively above and below the β sheet.

By using known modelling techniques, the crystal structure of the present invention can be used to produce a model for at least part of the Ras effector domain and/or at least part of the PI3K catalytic subunit.

As used herein, the term “modelling” includes the quantitative and qualitative analysis of molecular structure and/or function based on atomic structural information and interaction models. The term “modelling” includes conventional numeric-based molecular dynamic and energy minimisation models, interactive computer graphic models, modified molecular mechanics models, distance geometry and other structure-based constraint models.

The crystal structure of the present invention can be used to generate a structural model such as a three dimensional (3D) structural model (or a representation thereof). Alternatively, the crystal structure may be used to generate a computer model for the structure.

Molecular modelling techniques can be applied to the atomic co-ordinates of the PI3K/Ras complex to derive a range of 3D models and to investigate the structure of ligand binding sites. A variety of molecular modelling methods are available to the skilled person for use according to the invention [e.g Rational drug design: novel methodology and practical applications, ACS Symposium Series vol. 719 (Parrill & Reddy eds., 1991].

At the simplest level, visual inspection of a computer model of the PI3K/Ras complex can be used, in association with manual docking of models of functional groups into its binding pockets.

Software for implementing molecular modelling techniques may also be used. Suitable software and algorithms are generally available, and examples are described below.

Modelling may include one or more steps of energy minimisation with standard molecular mechanics force fields, such as those used in CHARMM and AMBER (see below).

These molecular modelling techniques allow the construction of structural models that can be used for in silico drug design and modelling.

In another aspect of the present invention, the above-mentioned models may be used to screen for or design ligands which are capable of binding the PI3K/Ras complex and/or which are capable of modulating the ability of PI3K and Ras to form a functional complex.

The screen may employ a solid 3D screening system or a computational screening system. Using these systems, test compounds may be screened to find those which interact spatially and preferentially with PI3K and/or Ras, through either computational or manual docking.

The molecular modelling steps used in the methods of the invention may use the atomic co-ordinates of a PI3K/Ras complex, and models derived therefrom, to determine binding surfaces. This preferably reveals van der Waals contacts, electrostatic interactions, and/or hydrogen bonding opportunities.

These binding surfaces will typically be used by grid-based techniques (see below) to map favourable interaction positions for functional groups. This preferably reveals positions in the PI3K/Ras complex for interactions such as, but not limited to, those with protons, hydroxyl groups, amine groups, hydrophobic groups (e.g. methyl, ethyl, benzyl) and/or divalent cations.

Once functional groups or small molecule fragments which can interact with specific sites in the binding surface of the PI3K/Ras complex have been identified, they can be linked in a single compound using either bridging fragments with the correct size and geometry or frameworks which can support the functional groups at favourable orientations, thereby providing a test compound according to the invention. Whilst linking of functional groups in this way can be done manually, perhaps with the help of software such as QUANTA or SYBYL, the following software may be used for assistance: HOOK [Molecular Simulations, Inc], which links multiple functional groups with molecular templates taken from a database, and/or CAVEAT [Lauri & Bartlett (1994) Comp. Aided Mol. Design 8:51-66], which designs linking units to constrain acyclic molecules.

Other computer-based approaches to de novo compound design that can be used with the PI3K/Ras atomic co-ordinates include LUDI [Böhm (1992) J. Comp. Aided Molec. Design 6:61-78; Molecular Simulations Inc], SPROUT [http://chem.leeds.ac.uk/ICAMS/SPROUT.html] and LEAPFROG [Tripos Inc].

A pharmacophore of PI3K and/or Ras can be defined i.e. a collection of chemical features and 3D constraints that expresses specific characteristics responsible for biological activity. The pharmacophore preferably includes surface-accessible features, more preferably including hydrogen bond donors and acceptors, charged/ionisable groups, and/or hydrophobic patches. These may be weighted depending on their relative importance in conferring activity [Computer-Assisted Lead Finding and Optimization (eds. Testra & Folkers, 1997].

Pharmacophores can be determined using software such as CATALYST (including HypoGen or HipHop) [Molecular Simulations, Inc], CERIUS², or constructed by hand from a known conformation of a lead compound. The pharmacophore can be used to screen in silico compound libraries, using a program such as CATALYST [Molecular Simulations, Inc].

Suitable in silico libraries include the Available Chemical Directory (MDL Inc), the Derwent World Drug Index (WDI), BioByteMasterFile, the National Cancer Institute database (NCI), and the Maybridge catalog.

Compounds in these in silico libraries can also be screened for their ability to interact with the PI3K/Ras complex by using their respective atomic co-ordinates in automated docking algorithms.

Suitable docking algorithms are described below.

Docking algorithms can also be used to verify interactions with ligands designed de novo.

As used herein, the term “test compound” means a compound which may be tested for its capacity to interact with PI3K and/or Ras and/or to modulate the ability of PI3K and Ras to form a functional complex, i.e. a complex which is capable of resulting in the activation of PI3K. The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a structural or functional mimetic, a peptide, a peptidomimetics, a derivatised test compound, a peptide cleaved from a whole protein, or a peptides synthesised synthetically (such as, by way of example, either using a peptide synthesiser or by recombinant techniques or combinations thereof, a recombinant test compound, a natural or a non-natural test compound, a fusion protein or equivalent thereof and mutants, derivatives or combinations thereof.

Thus, the structural information from the crystal structure of the present invention is useful in the design of potential ligands capable of interacting with the PI3K/Ras complex and/or capable of modulating the ability of PI3K and Ras to form a functional complex, and the models of the present invention are useful to examine the effect such a ligand is likely to have on the structure and/or function of Ras and for PI3K. The present invention also relates to a ligand identified using such methods.

As used herein, the term “ligand” refers to a test compound capable of binding to Ras and/or PI3K and/or the ras/PI3K complex. Preferably the ligand modulates the interaction between Ras and PI3K.

The ligands may be natural or synthetic. The term “ligand” also refers to a chemically modified ligand.

In one aspect, the identified ligand may act as a ligand model (for example, a template) for the development of other compounds.

As described above, the β-2 strand of Ras and the Rβ2 strand of PI3K are important to the interaction between PI3K and Ras. Advantageously, therefore, test compounds bind to or modulate the interaction of the β-2 strand of Ras and the Rβ2 strand of PI3K.

Test compounds and ligands which are identified with the crystal of the present invention can be screened in assays such as are well known in the art. Screening can be, for example in vitro, in cell culture, and/or in vivo. Biological screening assays preferably centre on activity-based response models, binding assays (which measure how well a compound binds to Ras and/or PI3K and/or a complex thereof), and bacterial, yeast and animal cell lines (which measure the biological effect of a compound in a cell). The assays can be automated for high capacity-high throughput screening (HTS) in which large numbers of compounds can be tested to identify compounds with the desired activity. The biological assay, may also be an assay for ligand binding activity a compound that selectively binds to Ras and/or PI3K and/or a complex thereof.

The ligands may be peptide ligands. Preferred peptide ligands are derived from PI3Kγ 220-270 or Ras regions 25-41 or 60-73.

Advantageously, the peptide ligands are provided in such a manner that the concentration thereof is selectively increased in the region of the plasma membrane, where PI3K is known to be significantly concentrated due to its intrinsic affinity for lipid membranes. For example, peptide ligands may incorporate a flexible membrane anchor, such that they are flexibly tethered to the membrane surface. Such a membrane anchor may be, for example, CAAX (C=Cys, A=aliphatic amino acid, X=any amino acid). This allows the inhibitory peptide to exploit the same membrane-concentrating effect as full-length PI3K, and allow peptides having even low affinities to bind Ras at the membrane. The use of CAAX for membrane localisation is known in the art and described, for example, in Kato et al., PNAS (1992) 89:6403-6407.

This embodiment is described in further detail in Example 3 below.

Peptides may be delivered to cells by any known means, including delivery by cellular localisation peptides such as the protein transduction domains of HIV Tat, HSV VP22 or Drosophila Antannapedia, as reviewed by Schwarze et al in Trends in Cell Biol. (2000) 10:290-295.

The ligand of the present invention may be used in a pharmaceutical composition, optionally together with a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical., compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the pharmaceutical composition is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose or chalk, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The invention further provides a method of preventing and/or treating disease in a mammal, the method comprising administering to a mammal a ligand or pharmaceutical composition of the present invention.

Typically, a physician will determine the actual dosage which will be most suitable for an individual subject and it will vary with the age, weight and response of the particular patient and severity of the condition. The dosages below are exemplary of the average case. There can, of course, be individual instances where higher or lower dosage ranges are merited.

The compositions (or component parts thereof) of the present invention may be administered orally. In addition or in the alternative the compositions (or component parts thereof) of the present invention may be administered by direct injection. In addition or in the alternative the compositions (or component parts thereof) of the present invention may be administered topically. In addition or in the alternative the compositions (or component parts thereof) of the present invention may be administered by inhalation. In addition or in the alternative the compositions (or component parts thereof) of the present invention may also be administered by one or more of: parenteral, mucosal, intramuscular, intravenous, subcutaneous, intraocular or transdermal administration means, and are formulated for such administration.

By way of further example, the pharmaceutical composition of the present invention may be administered in accordance with a regimen of 1 to 10 times, per day, such as once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The term “administered” also includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Hence, the pharmaceutical composition of the present invention may be administered by one or more of the following routes: oral administration, injection (such as direct injection), topical, inhalation, parenteral administration, mucosal administration, intramuscular administration, intravenous administration, subcutaneous administration, intraocular administration or transdermal administration.

Production of the Crystal

The crystals of the present invention may be prepared by expressing a nucleotide sequence encoding Ras and PI3K, by the use of a suitable host cell, and then crystahising the purified protein(s).

As used herein, the term “nucleotide sequence” refers to nucleotide sequences, oligonucleotide sequences, polynucleotide sequences and variants, homologues, fragments and derivatives thereof (such as portions thereof) which comprise the nucleotide sequences encoding Ras and PI3K amino acid sequences. The nucleotide sequence may be DNA or RNA of genomic or synthetic or recombinant origin which may be double-stranded or single-stranded whether representing the sense or antisense strand or combinations thereof. Preferably, the term nucleotide sequence is prepared by use of recombinant DNA techniques (e.g. recombinant DNA). The nucleotide sequence may include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vitro activity or life span of nucleotide sequences of the invention.

To produce a Ras/PI3K complex, it is preferable for the individual polypeptides to be expressed separately and mixed for crystallisation, due to the very fast off rate of the complex.

The sequences expressed may be wild-type or mutant sequences. The term “wild type” refers to the phenotype that is characteristic of most of the members of a species occurring naturally and which contrasts with the phenotype of a mutant species.

The term “mutant” refers to any organism that has undergone mutation or that carries a mutant gene that is expressed in the phenotype of that organism. A mutation may arise due to a substitution of one nucleotide for another or from a deletion of a nucleotide or an insertion of a nucleotide relative to a referenced wild type sequence. These single nucleotide variations are sometimes referred to as single nucleotide polymorphisms (SNPs). Some SNPs may occur in protein-coding sequences, in which case, one of the polymorphic forms may give rise to the expression of a defective or other variant protein and, potentially, a genetic disease. Other SNPs may occur in noncoding regions. Some of these polymorphisms may also result in defective protein expression (e.g., as a result of defective splicing). Other SNPs may have no phenotypic effects.

As used herein, the term “mutant” refers to polypeptide comprising any one or more changes in the sequence (the structural co-ordinates) and of the amino acid residues in Ras and/or PI3K.

As used herein, the term “variant” refers to Ras and/or PI3K or a portion thereof which may have deletions, insertions or substitutions of amino acid residues as long as the binding specificity of the molecules for each other in a Ras/PI3K complex is retained to the extent desired. In this regard, deliberate amino acid substitutions may be made on the basis of similarity in polarity, charge, solubility, hydrophobicity, hydrophilicity, and/or the amphipathic nature of the residues.

A wide variety of host cells can be employed for expression of the nucleotide sequences encoding the Ras and/or PI3K proteins of the present invention. These cells may be both prokaryotic and eukaryotic host cells. Suitable host cells include bacteria such as E. coli, yeast, filamentous fungi, insect cells, mammalian cells, typically immortalised, e.g., mouse, CHO, human and monkey cell lines and derivatives thereof. Preferred host cells are able to process the expression products to produce an appropriate mature polypeptide(s). Processing includes but is not limited to glycosylation, ubiquitination, disulphide bond formation and general post-translational modification.

The proteins comprising Ras and/or PI3K, produced by a host recombinant cell, may be secreted or may be contained intracellularly depending on the nucleotide sequence and/or the vector used. As will be understood by those of skill in the art, expression vectors containing the Ras and/or PI3K encoding nucleotide sequences can be designed with signal sequences which direct secretion Ras and/or PI3K coding sequences through a particular prokaryotic or eukaryotic cell membrane.

Other recombinant constructions may join the Ras and/or PI3K encoding sequence to nucleotide sequence encoding a polypeptide domain which will facilitate purification of soluble proteins (Kroll D J et al (1993) DNA Cell Biol 12:441-53). Such purification facilitating domains include, but are not limited to, metal chelating peptides such as histidine-tryptophan modules that allow purification on immobilised metals (Porath J (1992) Protein Expr Purif 3-0.26328 1), protein A domains that allow purification on immobilised immunoglobulin, and the domain utilised in the FLAGS extension/affinity purification system (Immunex Corp, Seattle, Wash.). The inclusion of a cleavable linker sequence such as Factor XA or enterokinase (Invitrogen, San Diego, Calif.) between the purification domain and the AR and PR is useful to facilitate purification.

Ras and/or PI3K may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences.

Preferably the fusion protein will not hinder the complexing capacity of Ras and/or PI3K.

After expression, the proteins may be purified and/or concentrated, for example by immobilised metal affinity chromatography, ion-exchange chromatography, and/or gel filtration. For crystals of the Ras/PI3K complex, Ras and PI3K fragments may be purified and allowed to dimerise in solution.

The protein(s) may be crystallised using any of the known techniques. Usually, in a crystallisation process, a crystallisation buffer is prepared with a lower concentration of a precipitating agent necessary for crystal formation. For crystal formation, the concentration of the precipitating agent has to be increased, by addition of precipitating agent or by diffusion of the precipitating agent between the crystallisation buffer and a reservoir buffer. Diffusion may be achieved by known techniques such as the “hanging drop” or the “sitting drop” method. In these methods, a drop of crystallisation buffer containing the protein(s) is hanging above or sitting beside a much larger pool of reservoir buffer. Alternatively, the balancing of the precipitating agent can be achieved through a semi-permeable membrane that separates the crystallisation buffer and prevents dilution of the protein into the reservoir buffer.

Generating the Crystal Structure

Once the crystals have been obtained, the structure may be solved by known Xray diffraction techniques. Many techniques use chemically modified crystals, such as those modified by heavy atom derivatization. In practice, a crystal is soaked in a solution containing heavy metal atom salts, or organometallic compounds, e.g., lead chloride, gold thiomalate, thimerosal or uranyl acetate, which can diffuse through the crystal and bind to the surface of the protein. The location(s) of the bound heavy metal atom(s) can the n be determined by X-ray diffraction analysis of the soaked crystal. The patterns obtained on diffraction of a monochromatic beam of X-rays by the atoms (scattering centers) of the crystal can be solved by mathematical equations to give mathematical coordinates. The diffraction data are used to calculate an electron density map of the repeating unit of the crystal. The electron density maps are used to establish the positions of the individual atoms within the unit cell of the crystal. Blundel, T. L. and N. L. Johnson, Protein Crystallography, Academic Press (1976).

Those of skill in the art understand that a set of structural coordinates determined by X-ray crystallography is not without standard error. For the purpose of this invention, any set of structure coordinates for Ras:PI3K complexes that have a root mean square deviation of protein backbone atoms of less than 0.75 Å when superimposed (using backbone atoms) on the structure coordinates listed in Table 3 shall be considered identical.

Modelling

The three dimensional structure of a new crystal may be modelled using molecular replacement. The term “molecular replacement” refers to a method that involves generating a preliminary model of a molecule or complex whose structure coordinates are unknown, by orienting and positioning a molecule whose structure coordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, N.Y., (1972).

Other molecular modelling techniques may also be employed in accordance with this invention. See, e.g., Cohen, N. C. et al., “Molecular Modelling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Using the structure coordinates of the Ras:PI3K crystal complex provided by this invention, molecular replacement may be used to determine the structure coordinates of a crystalline mutant or homologue of Ras and/or PI3K or of a related protein (such as a Ras variant).

Ligand Screening

One skilled in the art may use one of several methods to test compounds for their ability to associate with Ras and/or PI3K. This process may begin by visual inspection of, for example, a target site on the computer screen based on the structure coordinates given in Table 3. Selected test compounds may then be positioned in a variety of orientations, or docked, within an individual target site of Ras and/or PI3K as defined supra. Docking may be accomplished using software such as Quanta and Sybyl, followed by energy minimisation and molecular dynamics with standard molecular mechanics forcefields, such as CHARMM and AMBER.

Specialised computer programs may also assist in the process of selecting potential ligands. These include:

1. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (Miranker, A. and M. Karplus, “Functionality Maps of Binding Sites: A Multiple Copy Simultaneous Search Method.” Proteins: Structure. Function and Genetics, 11, pp. 29-34 (1991)). MCSS is available from Molecular Simulations, Burlington, Mass.

3. AUTODOCK (Goodsell, D. S. and A. J. Olsen, “Automated Docking of Substrates to Proteins by Simulated Annealing”, Proteins: Structure. Function, and Genetics, 8, pp. 195-202 (1990)). AUTODOCK is available from Scripps Research Institute, La Jolla, Calif.

4. DOCK (Kuntz, I. D. et al., “A Geometric Approach to Macromolecule-Ligand Interactions”, J. Mol. Biol., 161, pp. 269-288 (1982)). DOCK is available from University of California, San Francisco, Calif.

Once a ligand has been optimally selected or designed, substitutions may then be made in some of its atoms or side groups in order to improve or modify its binding properties.

Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analysed for efficiency of fit to Ras and/or PI3K by the same computer methods described above.

### EXAMPLES

The following examples serve to illustrate the present invention, but should not be construed as a limitation thereof. The invention particularly relates to the specific embodiments described in these examples.

Experimental Procedures

Protein Cloning and Expression

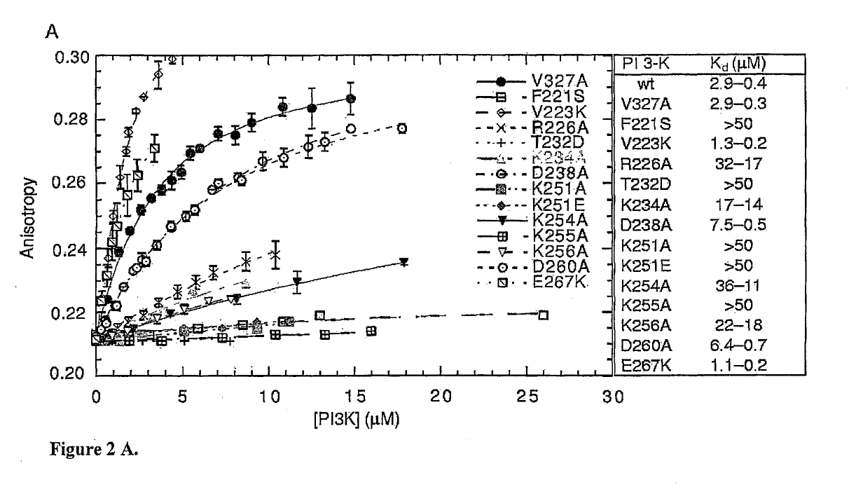

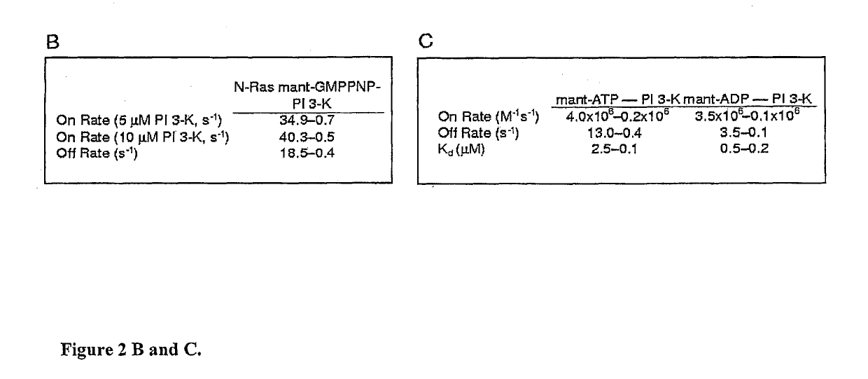

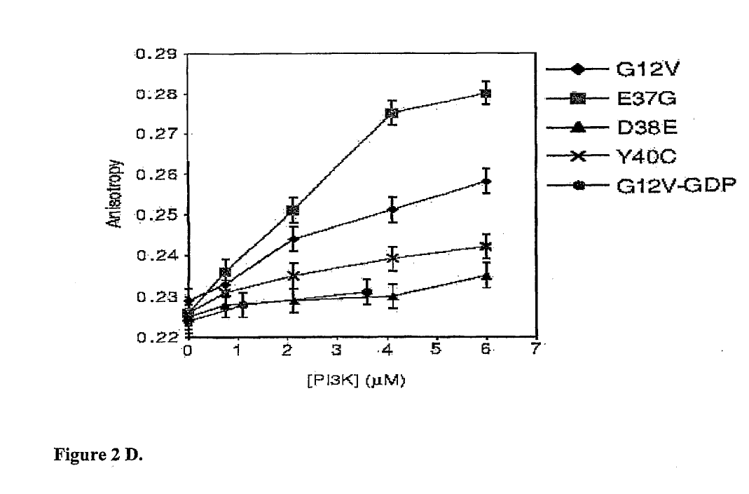

For crystallography, equilibrium binding, and kinetic measurements, a construct containing residues 144-1102 of the human PI3Kγ with a C-terminal His₆ tag was cloned into pVL1393 (InVitrogen) for baculovirus expression. The human enzyme has 1102 residues, not 1101 as indicated by the GenBank sequence P48736. The error in the database sequence is due to omission of an Ala following Ala-29. To make the RBD mutants, we used a cassette containing a V327A mutation, which encoded a NheI site used for subcloning. This V327A “pseudo wild type” construct has the same affinity for N-Ras as the wild type protein (FIG. 2A). The DASAA PI3Kγ mutant used for the in vitro and in vivo experiments contains the T232D, K251A, K254S, K255A, and K256A mutations in the context of the fall-length pseudo wild type p110γ subunit. For expression in COS-7 cells, the DASAA mutant was subcloned into pcDNA3 with no tags. For in vitro Ras activation studies, the DASAA mutant was subcloned into pVL1393 with a C-terminal His₆ tag.

PI3K constructs in pVL1393 were transfected into Sf9 cells with BaculoGold DNA (Pharmingen). Infected cells were incubated at 27° C. for 48 hrs. Harvested cells were washed in PBS, pelleted, frozen in liquid nitrogen, and stored at −80° C.

The DNA sequence encoding residues 1- 166 of H. sapiens H-Ras G12V was cloned as a NdeI/BamHI fragment into pET11a (Novagen) and transformed into C41([)E3) cells. For expression, bacteria were grown at 37° C. to an O.D.₆₀₀ of 0.7, and induced with 100 μM IPTG for 12 hours. Full-length, his-tagged H-Ras mutants (a gift from J. Downward) were grown in a similar manner and induced for 5 hours with 500 μM IPTG.

In Vitro and in Vivo Activity Measurements

H-Ras was loaded with GTPγS or GDP by incubation in exchange buffer (50 mM Tris pH 7.5, 40 mM EDTA, 200 mM (NH₄)₂SO₄) with a 20-fold molar excess of the nucleotide. The protein was gel-filtered on a PD-10 column (Pharmacia) and frozen at −80° C.

In vitro assays were carried out in 10 μL of ice-cold reaction buffer (0.12 M NaCl, 25 mM HEPES pH 7.5, 1 mM EGTA, 1 mM DTT, 1 mg/mL BSA, 1% betaihe (0.02% w/v) and 1% Tween 20) with 1 μM lipid-modified, full length H-Ras loaded with GTPγS or GDP, and 300 ng of p110γ/p101 complex. All assays contained 5 μg neutrophil memnbranes and 5-10 μCi of γ-³²P ATP. The reaction was ran for 16 minutes, quenched, and the lipids were extracted, deacylated and quantitated by HPLC as described (Stephens et al., 1991).

COS-7 cells were transfected with plasmid DNA (5 μg full-length PI3Kγ, 5 μg myc-tagged p101, 5 μg H-Ras G12V, or an unrelated control DNA for a total of 15 μg per transfection in a final volume of 0.5 mL) as described previously (Stephens et al., 1997). After electroporation, the cells were incubated for 48 hours, washed in HEPES-buffered DMEM containing 1 mM NaHCO₃ and 0.2% fatty acid-free BSA, and serum-starved for a further 10 hours. The cells were washed with phosphate-free medium and incubated for 60 minutes with 300-500 μCi γ-³²P ATP per dish. The cells were harvested and ³²P-labeled phospholipids measured as above.

Protein Purification

Sf9 cells from 0.9 L of culture were sonicated in buffer (50 mM KH₂PO₄, 10 mM Tris, pH 8, 0.1 M NaCl, 1 mM MgCl₂) containing protease inhibitors (complete EDTA-free tablets, Boehringer Mannheim/Roche). After centrifugation, the supernatant was incubated with Talon resin (Clontech) for an hour. The resin was washed with 100 mL of solution 1 (50 mM KH₂PO₄ pH 8.0, 20 mM Tris, pH 8, 0.1 M NaCl, 1% betaine, 0.05% Tween 20), 125 mL of solution 2 (50 mM KH₂PO₄ pH 7.0, 20 mM Tris pH 7.5, 0.1 M NaCl, 1% betaine, 0.05% Tween 20), and 50 mL of solution 3 (20 mM Tris, pH 7.5, 10 mM imidazole, 0.1 M NaCl, 1% betaine, 1% ethylene glycol). Protein was eluted with 30 mL of elution buffer (20 mM KH₂PO₄, pH 5.6, 0.1 M EDTA, 1% betaine, 1% ethlyene glycol, and 0.02% CHAPS). The protein was loaded onto a HiTrap SP column (Phannacia), washed with 0.1 M NaCl in 20 mM KH₂PO₄, pH 5.6, 1 mM DTT and eluted with 0.45 M NaCl. The protein was concentrated to 1 mL (Ultrafree-4, 30K cutoff filter, Millipore) and loaded onto a PD-10 colurnn equilibrated in gel filtration buffer (20 mM Tris pH 7.2, 50 mM (NH₄)₂SO₄, 1% betaine, 1% ethylene glycol, 0.02% CHAPS, 5 mM DTT), concentrated to 4-6 mg/mL and snap frozen.

H-Ras 1-166 was extracted from 1 L of bacterial culture with B-PER reagent (Pierce) and purified by two passages over a HiLoad Q column (Pharmacia) with a salt gradient (0-1 M KCl in 50 mM Tris pH 7.5, 1 mM MgCl₂, 5 μM GDP, and 1 mM DTT). The protein was gel filtered on a Superdex 75 column (Pharmacia) in storage buffer (50 mM Tris pH 7.5, 1 mM MgCl₂, 5 μM GDP, and 1 mM DTT), concentrated to 20 mg/mL, and snap frozen. Full-length H-Ras mutants were purified as previously described (Rodriguez-Viciana et al., 1997).

To load the H-Ras with GMPPNP, 200 nmol of protein were mixed with 3 μmol of GMPPNP (Sigma) in 1 mL of Ras loading buffer (50 mM Tris pH 8.5, 1 mM EDTA, 200 mM (NH₄)₂SO₄). The mixture was incubated for 1 hour with alkaline phosphatase (2 μL) (Boehringer Mannheim) and the exchange stopped by the addition of MgCl₂ to 50 mM. H-Ras was gel filtered on a Superdex 75 column in 20 mM Tris, pH 7.5, 2 mM MgCl₂, and 1 mM DTT. The protein was concentrated to ˜12 mg/mL and snap frozen.

Anisotropy Titrations

Mant-nucleotides were synthesised as described previously (Jameson and Eccleston, 1997). N-Ras and H-Ras mutants were loaded with mant-GMPPNP as in (Moore et al., 1993), gel filtered into running buffer (20 mM Tris pH 7.5, 2 mM MgCl₂, 1 mM DTT), and frozen at −80° C. The concentration of Ras.mant-GMPPNP was determined by measuring the absorbance of the mant group at 350 nM with ε=5700 M⁻¹ cm⁻¹ (Hiratsuka, 1983). For binding assays, the 144-1102 human PI3Kγ was titrated into a 1 μM solution of fill length, unmodified N-Ras.mant-GMPPNP. Data were collected with SLM 8000 S and ISS PC1 spectrofluorimeters, set up in the T-format and L-format, respectively, with prism polarizers. All measurements were taken at 20° C. with a λₑₓ of 366 nm. Both emission PMTs were fitted with 399 nm cutoff filters. Five measurements of each data point were obtained and averaged. Data were fitted to the binding equation.

Stopped Flow Anisotropy

A Hi-Tech MX61 instrument (Hi-Tech, Salisbury) set up in the T-format with prism polarizers was used to collect data at 20° C. The mant group was excited at 366 nm and emission monitored through 399 nm cutoff filters. The association kinetics were followed by rapid mixing of PI3Kγ with 1 μM N-Ras.mant-GMPPNP under pseudo first-order conditions (PI3Kγ in large excess). Specifically, equal proportions of N-Ras and PI3Kγ were mixed to a final Ras concentration of 1 μM and final PI3Kγ concentrations of 5 and 10 μM. The dissociation rate was measured by mixing a solution of 1 μM N-Ras.mant-GMPPNP and 5 μM PI3K with 9 μM N-Ras.GMPPNP. Attempts to determine association rates at higher PI3K concentrations were limited by solubility of the protein. Data were fitted to single exponential curves within the Hi-Tech software.

Steady State and Kinetic Measurements of ATP and ADP Binding

Equilibrium binding of mant-ATP to PI3K was measured in a SLM 8000 S spectrofluorimeter with λₑₓ=280 nm and λₑₓ=440 nm. This allowed monitoring of energy transfer from tryptophan in the PI3K to mant-ATP. Mant-ATP was titrated into a 1 μM solution of PI3K in running buffer. Each data point represents the average of data collected for 30 seconds with a 1 second integration time. For data collected in the presence of Ras, the solution also contained 6 μM H-ras.GMPPNP. The data were fitted to the binding equation with Kaleidagraph.

All kinetic data were collected in a Hi-Tech Na61 instrument set up in the single push mode with λₑₓ=280 nm and emission monitored with 399 nm cutoff filters. A solution of PI3K was mixed a solution of mant-ATP such that the final PI3K concentration was 1 μM and the ATP concentrations were 2.5, 5. 7.5, 10, 12.5, and 25 μM. The displacement experiment was carried out by mixing 1 μM PI3K and 2 μM mant-ATP with 50 or 200 μM ATP. Curve fits were carried out with the Hi-Tech software and Kaleidagraph. Equilibrium and kinetic data for mant-ADP were collected by the same methods.

Crystallography

H-Ras.GMPPNP and PI3Kγ (V223K in the context of the 144-1102 pseudo wild type construct) were mixed to final concentrations of 170 μM for H-Ras and 29 μM for PI3K. The mixture was incubated for 3 days at 17° C., and then set in hanging drops against a reservoir of 50 mM KH₂PO₄/K₂HPO₄ pH 6.3, 4.3% PEG 8000. Crystals were propagated by hair seeding and grew to dimensions of 120 μM×70 μM×50 μM in 10 days. SDS-PAGE analysis of the crystals showed that the PI3Kγ had been completely digested into four fragments (43, 32, 20, and 10 kDa) during the time course of crystallisation. The largest crystals were transferred to cryoprotectant (reservoir with 40% trehalose) in two steps, each lasting thirty seconds, and frozen in a liquid N₂ cryostream.

Data sets were collected at beamlines ID14-2 and ID14-1 at the European Synchotron Radiation Facility in Grenoble at a wavelength of 0.934 Å. The space group was P3₁21 with cell dimensions of a=b=113.6 Å, c=183.9 Å, α=β=90°, γ=120°, and one complex in the asymmetric unit. The reflections from two crystals were indexed and integrated with MOSFLM (Leslie, 1992), merged, and scaled with SCALA (CCP4, 1994). Molecular replacement was carried out with AmoRe. The initial model consisted of our PI3Kγ structure (PDB:1qmm). A second rotation/translation search with the structure of Ras (Pai et al., 1990) (PDB:5p21) located the Ras molecule. The partial model was rebuilt in O (Jones et al., 1991) and refined with CNS (Brunger et al., 1998). The average B factor is 70 Å². No residues are in the disallowed regions of the Ramachandran plot. Figures were prepared with MOLSCRIPT (Kraulis, 1991), GRASP (Nicholls et al., 1991), and Raster3D (Merritt and Bacon, 1997).

In the structure, all residues in the Ras are visible. As in the previous structure of the free PI3Kγ, several loops are not visible in the electron density. These include the RBD-C2 domain linker (residues 323-352), CBR3 in the C2 domain (residues 436-456), the loop between C2β7 and C2β8 (residues 489-497), the activation loop in the catalytic domain (residues 969-980), and residues 1085 to 1102 in the C-terminus. Several loops that were defined in the free PI3K are not visible in the electron density of the complex. These include CBR1 (residues 375-378), and loops between residues 754-760, residues 896-901, and residues 1038-1046. It is possible that some of the disordered loops not visible in the electron density also correspond to sites of proteolytic nicking (see above).

The N-terminal half of the helical domain (residues 527-620, containing helices hA1 to hB2′) is not present in the structure. Since the Ras of a symmetry-related complex packs in this space, and SDS-PAGE analysis of the crystals showed no full-length PI3Kγ, it appears that this half of the domain was lost to proteolysis during crystallisation.

The ordering of the 255-267 loop and better side-chain definition at places in the region from 228 to 255 showed that there was a register shift in the fit of the sequence to the electron density in the original structure of PI3K in the absence of Ras.

### Example 1

Ras/PI3K Interaction

Ras.GTP Directly Activates PI3Kγ

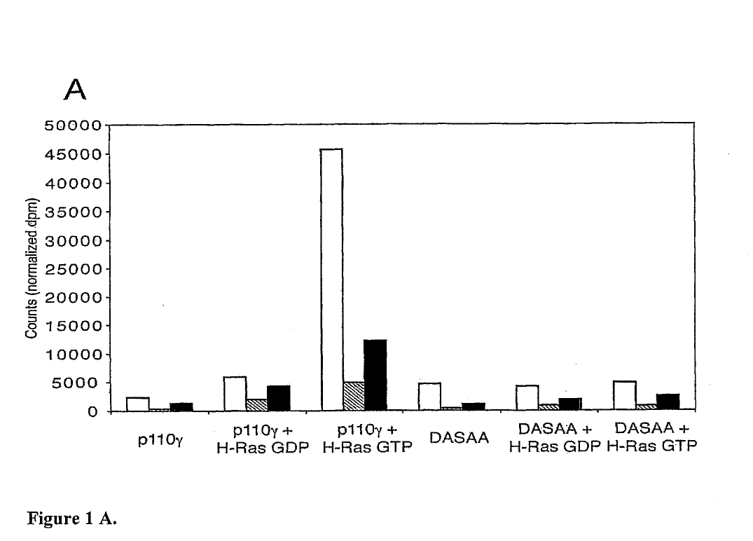

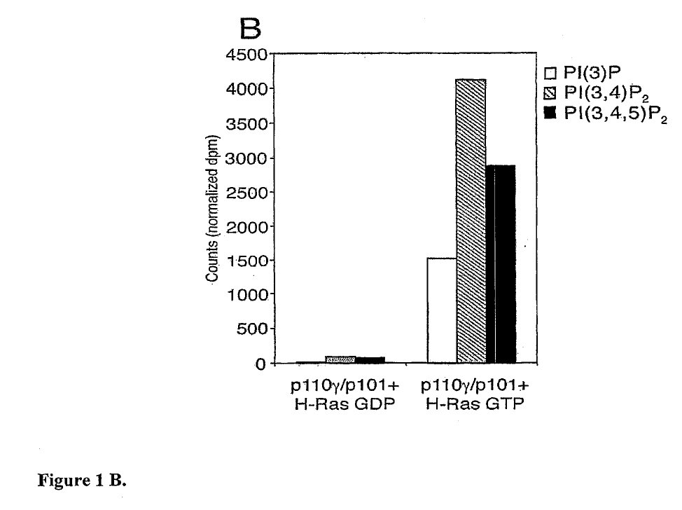

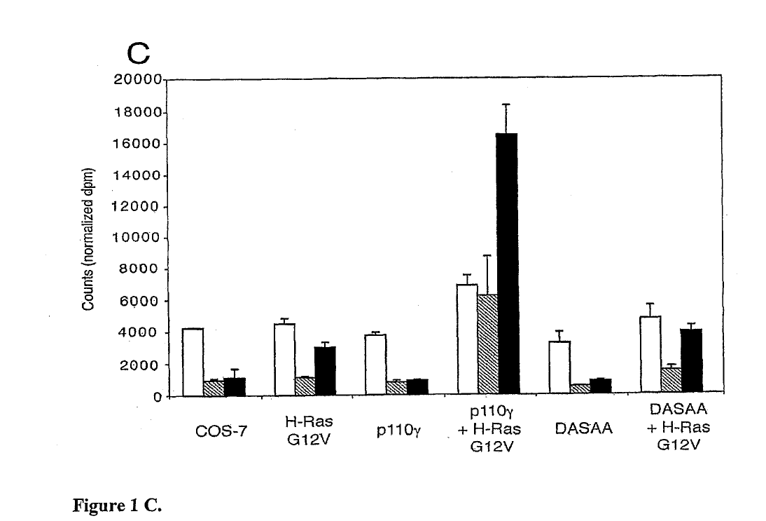

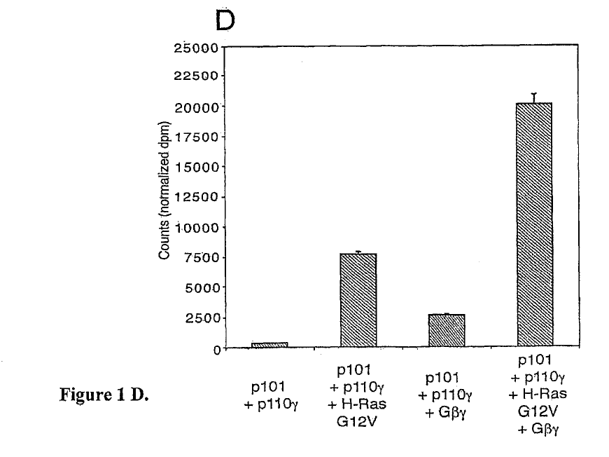

PI3Ks are able to integrate and amplify signals received from multiple signalling pathways. Tyrosine kinases and Ras synergistically activate PI3Kα (Rodriguez-Viciana et al., 1996b), while tyrosine kinases and the Gβγ subunits of heterotrineric G proteins synergistically activate PI3Kβ (Kurosu et al., 1997). Although binding of Gβγ strongly stimulates PI3Kγ (Stephens et al., 1997; Maier et al., 1999), the functional significance of Ras binding to PI3Kγ and its relationship with Gβγ activation have not been established. To determine the consequences of Ras binding to PI3Kγ, we investigated the effect of full-length, lipid modified, constitutively activated H-Ras (G12V) on PI3Kγ activity in vitro, using isolated neutrophil membranes. Our results indicate that H-Ras.GTP causes a 170-fold increase in the level of PtdIns(3,4)P₂ and a 48-fold increase in PtdIns(3,4,5)P₃ for the p110γ/p101 heterodimer (FIG. 1A). This activation is strictly dependent on Ras.GTP, and greatly exceeds previously reported stimulation of p110α/p85 heterodimer by Ras (Rodriguez-Viciana et al., 1996b). The p110γ subunit alone is also stimulated by H-Ras.GTP with a 3-fold and a 4-fold increase in the production of PtdIns(3,4)P₂ and PtdIns(3,4,5)P₃, respectively (FIG. 1A). Based on our structure of a complex of PI3Kγ with Ras (see below) we constructed a p110γ variant in which several residues essential for Ras binding were mutated. This multiple mutant (DASAA) has the same basal activity as the wild type enzyme in vitro, but is not activated by H-Ras.GTP (FIG. 1A). These results demonstrate that H-Ras directly activates PI3Kγ.

To demonstrate Ras activation of PI3Kγ in vivo, and its relationship to Gβγ stimulation, we transfected COS-7 cells with H-Ras (G12V), full-length p110γ, and p101. As shown in FIG. 1B, H-Ras stimulated the PI3Kγ activity by 4- to 7-fold. The level of activation is similar to the Ras activation of PI3Kα in COS-7 cells (Rodriguez-Viciana et al., 1994). Furthermore, Gβγ and Ras synergistically stimulate PI3Kγ activity (FIG. 1B). As in the in vitro experiments, Ras stimulates PI3Kγ activity in the absence of the p101 subunit (FIG. 1B). This stimulation of PI3Kγ by Ras is considerably greater than previously reported (Rubio et al., 1997) and may be due to differences in the constructs used. We have used a full length p110γ, whereas the previous work used a deletion variant. The previous work also used p110γ in the absence of p101. Our work shows that p101 is not essential for Ras activation, but greatly augments it.

PI3Kγ is abundant in neutrophils, and is essential for the production of reactive oxygen species (respiratory burst) that have an important role in the inflammatory response of these cells (Hirsch et al., 2000; Li et al., 2000; Sasaki et al., 2000). PI3Kγ activation by N-formyl-methionine-leucine-phenylalanine (fMLP) proceeds through G protein coupled receptors, which activate heterotrimeric G proteins. fMLP stimulation of neutrophils also causes a rapid activation of Ras (Worthen et al., 1994; Coffer et al., 1998). Because Ras activates PI3Kγ, it is possible that Ras and Gβγ synergistically participate in the fMLP-induced, PI3Kγ-dependent respiratory burst in neutrophils.

PI3Kγ Forms a Transient Complex with Ras.mant-GMPPNP

We characterised the solution binding of PI3Kγ to Ras by observing changes in the fluorescence anisotropy of full length, unmodified N-Ras.mant-GMPPNP (mant, 2′- (or -3′)-O-(N-methylanthraniloyl)). The K_{d} for wild type PI3Kγ and N-Ras.mant-GMPPNP is 2.8±0.4 μM. The K_{d} for wild type H-Ras (3.2±0.5 μM) is nearly identical to the K_{d} for N-Ras. This dissociation constant is higher than the 160 nM K_{d} for the Ras.Raf RBD complex (Herrmann et al., 1995; Sydor et al., 1998) and the 1 μM K_{d} for the Ras.RalGDS RBD complex (Herrmann et al., 1996). It is also approximately ten fold higher than the previously published dissociation constant for PI3Kγ and Ras (Rubio et al., 1999). The discrepancy between our results and those reported previously for p110γ may be due to differences in the constructs and methods used to determine the dissociation constants. The previously published work used pull-down assays with an immobilised GST-PI3Kγ construct.

Although the solution K_{d} for the PI3Kγ.Ras complex is high, it would be sufficient for interaction of the two proteins on lipid membranes. A similar situation is apparent for other signalling complexes that are active at membrane surfaces. For example, the estimated affinity of phospholipase Cβ₂ (PLCβ₂) for the Gβγ subunits in the bulk phase is about 3 μM, yet Gβγ is one of the principal activators of PLCβ₂ activity in cells (Runnels and Scarlata, 1998). Because PLCβ and Gβγ subunits have high affinities for membrane, these signalling components become concentrated at lipid membranes. The result of this concentration is that even fairly weak interactions lead to lateral association on the membrane. PI3Kα and PI3Kγ ((Barnett et al., 1995) and L. Stephens, unpublished data) have been shown to strongly bind to phosphoinositide-containing lipid membranes even in the absence of any other protein components. Consequently, the bulk affinities that we measure for soluble Ras translates into a significant interaction with membrane-localised Ras.

Dynamic studies of PI3Kγ and N-Ras indicate both rapid association and dissociation of the two proteins (FIG. 2B). While the dissociation rate of the complex is similar to that of Ras.Raf RBD, the association rate of PI3Kγ.Ras is an order of magnitude slower (Sydor et at., 1998). The association rate constant of the Ras.PI3K complex (18 s⁻¹) is two orders of magnitude higher than the catalytic turnover rate of PI3Kγ, which is about 0.1 s⁻¹ for PtdIns(3,4,5)P₃ (Maier et al., 1999). As with the Ras.Raf complex, the “activating” interaction of PI3Kγ with Ras is limited by the dissociation of the Ras.effector complex and not by the catalytic cycle of the effector enzyme. The transience of the complex ensures that PI3Kγ is not constitutively activated.

### Example 2

Crystallographic Studies

Structure of a Ras.PI3Kγ Complex

We reasoned that the transience of the Ras.PI3Kγ complex might be an impediment to the determination of its structure. Using a predicted model of the interaction of PI3Kγ with Ras (Walker et al., 1999), we constructed two tighter-binding PI3K mutants, V223K and E267K, for co-crystallisation with Ras. The K_{d}s for Ras.GMPPNP of these and other mutants discussed below are shown in FIG. 2A. Neither the wild-type protein, the pseudo wild-type (V327A, see methods), nor the V223K mutant exhibit detectable binding to N-Ras.GDP, consistent with the role of PI3Kγ as an effector. With the V223K mutant we obtained crystals of the Ras.PI3Kγ complex.

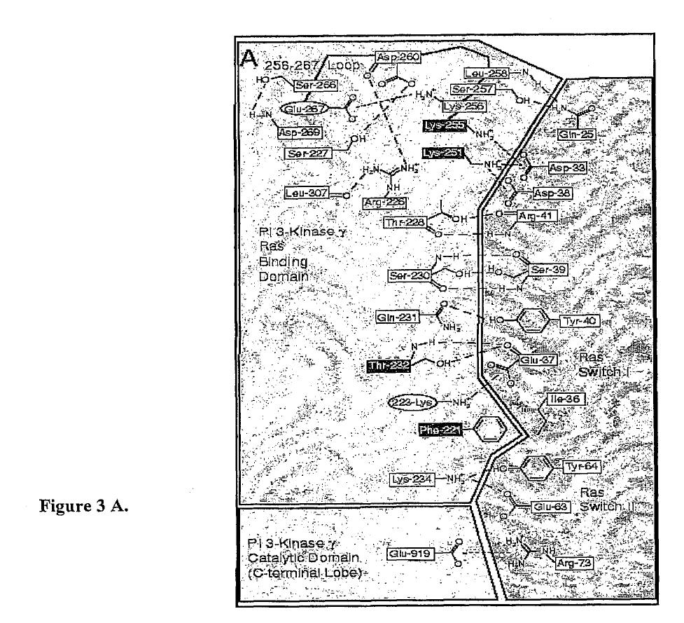

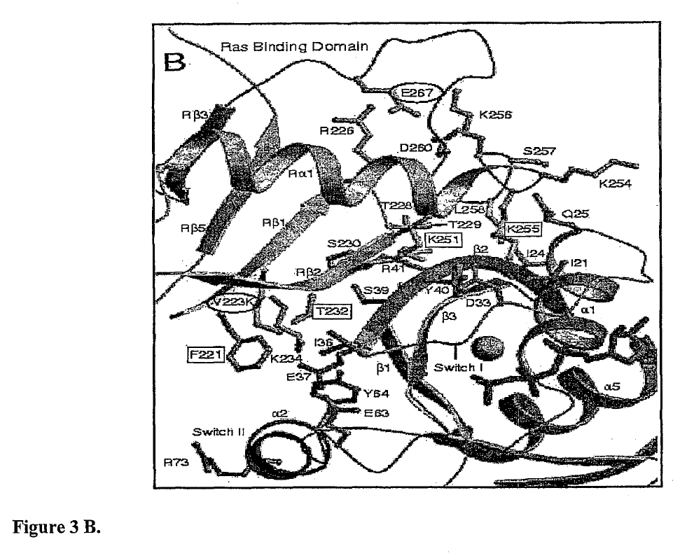

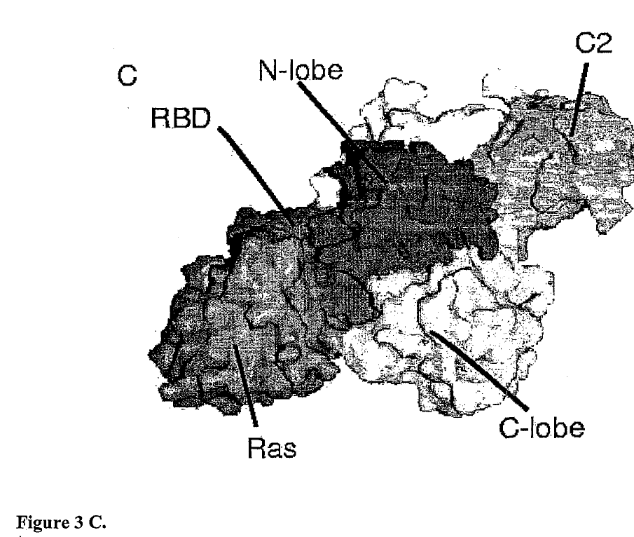

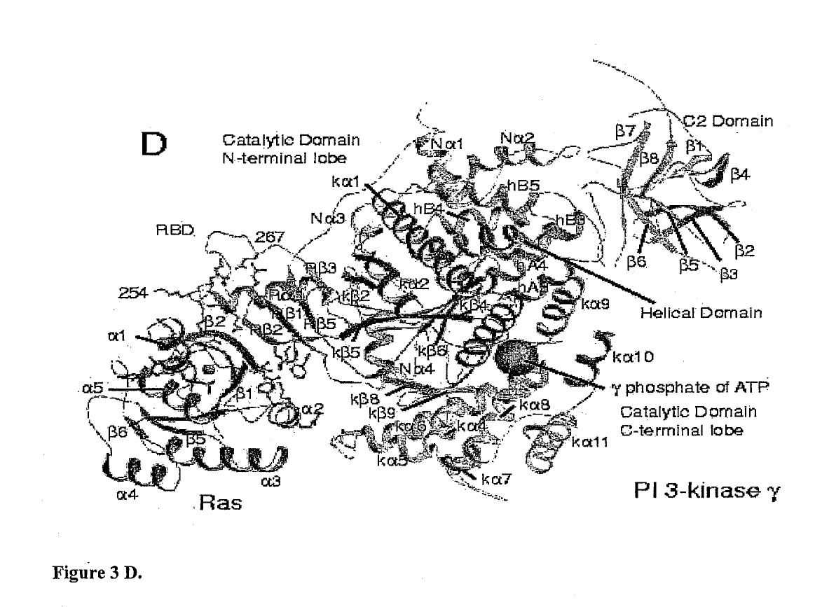

The overall organisation of the PI3Kγ catalytic subunit (Walker et al., 1999), which contains a Ras-binding domain (RBD), a C2 domain, a helical domain, and a catalytic domain, is preserved in the structure of the complex (FIG. 3). PI3Kγ interacts with many of the switch I residues of Ras with which the downstream effectors Raf and RalGDS also interact. However, a unique feature of the PI3Kγ.Ras complex is that the switch II region is also involved. Interactions with Ras switch II residues have previously only been observed in complexes of Ras with its upstream regulators, GAPs (Scheffzek et al., 1997) and GEFs (Boriack-Sjodin et al., 1998).

The most striking feature of the interaction with Ras is the ordering of a loop, residues 255 to 267, in the RBD (FIG. 3B), that is not visible in the free PI3Kγ structure, but is clearly defined in the electron density of the complex. This loop appears to act as a wedge that drastically rotates Ras with respect to Ras bound to other effectors, so that it establishes novel switch II contacts with the PI3Kγ RBD and catalytic domain.

Ras Binding Induces Ordering of a Critical Loop at the RBD.Ras Interface

The entropic cost of locking the PI3Kγ 255-267 loop into a single conformation might be offset by interactions formed between residues in the loop, and between the loop and the rest of the RBD. Eliminating these interactions or providing additional ones would affect the stability of the loop, and concomitantly decrease or increase the affinity of Ras for the kinase. In support of this hypothesis, the PI3Kγ mutants R226A, K256A, and D260A all eliminate hydrogen bonds or salt bridges that stabilise the 255-267 loop (FIG. 3A), and reduce binding by up to an order of magnitude (FIG. 2). The side chain of another attenuating mutant, Lys-254, points away from the Ras. Nevertheless, mutation of this residue to Ala greatly lowers the affinity of PI3Kγ for Ras, possibly by permitting more flexibility in the turn containing the critical residues Lys-255 and Lys-256. The complex-stabilising E267K mutant would permit the formation of an additional hydrogen bond from the mutant Lys-267 to the backbone amide oxygen of Phe-249, which could further stabilise the 255-267 loop and helix Ral. Apparently this also offsets any destabilisation that arises from placing Lys-267 and Lys-255 fairly near each other on the surface of the protein.

A predicted model of Ras bound to PI3Kγ (Walker et al., 1999) was based on the structure of the RalGDS RBD bound to Ras. In the current structure, with the 255-267 loop ordered and in place, steric clashes with this loop would prevent Ras from binding as predicted by our previous model. Instead, this loop wedges between Ras and the remainder of the RBD to rotate Ras toward the catalytic domain. The 255-267 loop is probably the primary determinant of the novel switch II contacts characteristic of the Ras.PI3K interaction (see below).

The Switch I Region of Ras Forms Typical Effector Interactions with PI3K

At the interface between Ras and the PI3K RBD, strand β2 of Ras is aligned with PI3K strand Rβ2 (FIG. 3B). This produces a continuous antiparallel β-sheet between the Ras and the RBD. The interaction is common to structures of Rap1A and Ras in complexes with Raf-RBD and RalGDS-RBD, respectively (Huang et al., 1998; Vetter et al., 1999). Most of the interactions between Ras and PI3Kγ are either hydrogen bonds or salt bridges (FIG. 3A), in agreement with the observation that the affinity of the two proteins is inversely related to the ionic strength of the buffer (data not shown). The area of the interface between the PI3Kγ RBD and Ras (1308 Å²) is similar to the surface areas of the Raf RBD (1333 Å²) and RalGDS RBD (1331 Å²) interfaces.

The effects of several PI3Kγ mutations that we made in the RBD.Ras interface are consistent with their apparent roles in forming switch I contacts. The T232D, K251A/E, and K255A mutations all eliminate binding by removing hydrogen bond donors or acceptors from the Ras switch I.PI3K interface, and in the case of the T232D and K251E mutations, by putting negatively charged PI3Kγ side chains into proximity with negatively charged side chains in Ras (FIG. 3A). K251E is the equivalent of the K227E mutant in PI3Kα, which also eliminates Ras binding (Rodriguez-Viciana et al., 1996b; Bondeva et al., 1998). The mechanism of the tighter binding V223K mutant can be explained in terms of interaction with the switch I region, because this mutation permits formation of an additional hydrogen bond between the RBD Lys-223 and Ras Glu-37.

Ras Makes Essential Interactions with PI3K using its Switch II Region

Unlike any other effectors downstream of Ras, PI3K makes critical interactions with the switch II region. Although early structural studies of Ras showed that both switch I and switch II regions of Ras changed conformation upon GTP binding and transforming mutants of Ras involving both of these regions have been reported, the structures of other effector RBDs in complexes with Ras showed only switch I interactions. In the PI3K complex, we observe extensive switch II contacts that are probably the consequence of the unique orientation of Ras relative to the RBD of PI3K. We have generated two mutants involving residues making switch II contacts, F221S and K234A. F221S eliminates hydrophobic interactions between PI3Kγ Phe-221 and Tyr-64 in switch II. Mutation of Lys-234 to Ala probably increases the K_{d} These mutants confirm the essential role of Ras Switch II in binding PI3Kγ. by eliminating hydrogen bonds to switch II residues Glu-63 and Tyr-64 (FIG. 3A). In addition, the Y64G Ras mutation abolishes a class IA PI3K binding (Moodie et al., 1995), suggesting that interaction with the Ras switch II is probably a general characteristic of PI3K.Ras complexes.

The PI3K Catalytic Domain Makes Direct Interactions with Switch II of Ras

Intriguingly, Ras directly contacts the C-terminal lobe of the PI3Kγ catalytic domain. Arg-73 in the switch II region of Ras is near enough to form a salt bridge with Glu-919 in the turn between helices kα5 and kα6. It was predicted that Arg-73 would be important in the interaction of Ras with other proteins (reviewed in (Polakis and McCormick, 1993), The structure of Ras with Sos confirmed the critical role of this residue in the interaction of Ras with nucleotide exchange factors (Boriack-Sjodin et al., 1998). This direct contact between the PI3K catalytic domain and Ras may be the consequence of the 255-267 loop of PI3K acting as a wedge to rotate the Ras toward the catalytic domain, and may be essential for induction of conformational change in the PI3Kγ (see below). This interaction may also be a feature of the Ras binding to PI3Kα, which has an even larger wedging loop in the location analogous to 255-267 of PI3Kγ.

Ras Binds to RBDs in Effector-Specific Orientations

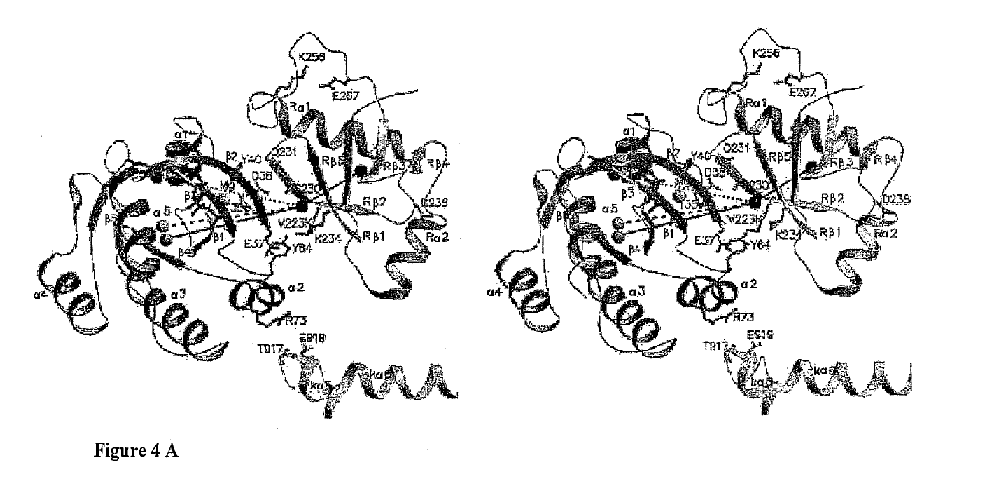

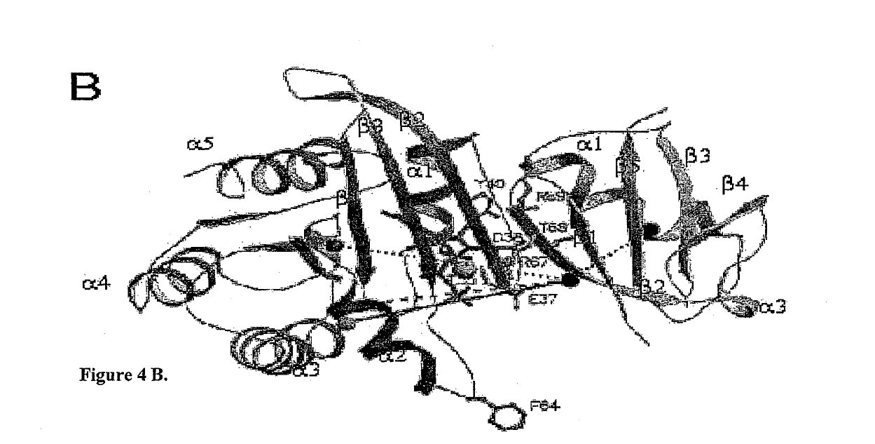

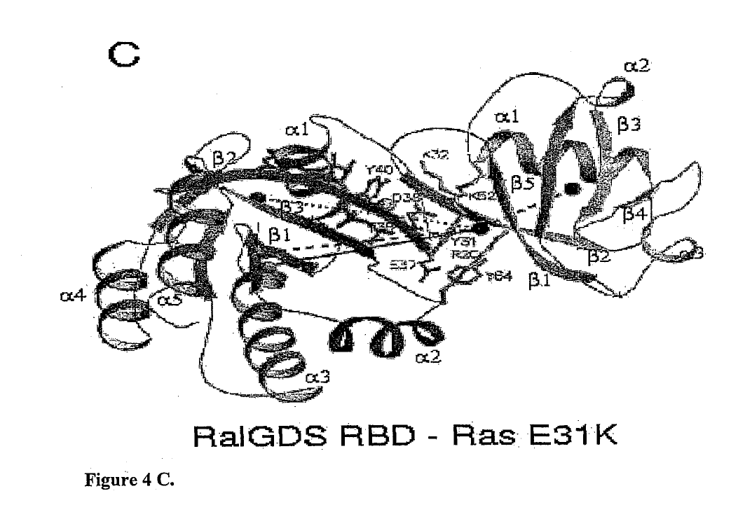

Two structures of isolated RBD complexes with Ras or the Ras-like protein Rap1A have been previously solved. In FIG. 4, the Raf RBD.Rap1A complex (Nassar et al., 1995) and the RalGDS RBD.Ras complex (Huang et al., 1998) are compared with the PI3Kγ.Ras complex.

All three Ras.RBD complexes use a similar general mode of Ras-effector interaction, in which a β-sheet in the Ras and a β-sheet in the RBD are aligned to form a single β-sheet connecting the two proteins. Contacts between the switch I region of Ras and the RBD stabilise the interaction and ensure its dependence on Ras-GTP.

Despite the common mode of the Ras-RBD interaction, Ras discriminates among its effectors by rotating with respect to the RBD of a given effector. Ras bound to PI3Kγ is rotated by 35° relative to the centroid of Rap1A bound to the Raf RBD, and by 17° relative to Ras bound to the RalGDS RBD (FIG. 4A). In PI3Kγ, the length of helix Rα1 and the size of the subsequent loop (255-267) cause the profound rotation of Ras relative to the PI3Kγ RBD and the switch II interactions, for which there are no equivalents in the other Ras effector RBDs. The 255-267 loop appears larger in PI3Kγ and α than in PI3Ks β and δ. This is in agreement with the observation that PI3Kδ appears to differ from PI3Kα in its interaction with Ras (Kinashi et al., 2000).

Structural Insights Into Signalling Pathway-Specific Ras Mutations

Five Ras mutants have been defined that differentially affect Raf, RalGDS and PI3Kα signalling pathways. These mutants, T35S, E37G, D38E, Y40C and Y64G, have served as useful tools for unravelling networks of signal transduction pathways (White et al., 1995; Rodriguez-Viciana et al., 1997). The selective activation displayed by the switch I mutants (T35S, E37G, D38E and Y40C) appears to be due to subtle differences in the network of interactions that the switch I region makes in the three Ras/effector complexes. For the switch II mutant (Y64G), PI3K activation is probably selectively abrogated simply because PI3K forms a switch II contact whereas the other effectors do not (Vetter et al., 1999).

E37G is a Ras mutant that activates RalGDS but not Raf Although this mutant does not bind to or activate PI3Kα, it was recently shown that it does activate PI3Kδ (Kinashi et al., 2000). We find that, as with PI3Kδ, PI3Kγ binds Ras E37G similarly to wild type Ras (FIG. 2D). Glu-37 of Ras forms only a weak ionic interaction with, RalGDS, while it forms two hydrogen bonds with the guanidinium group of Arg-59 in Raf. Although we see a salt link between PI3Kγ Lys-223 and Ras Glu-37 in the structure of the V223K mutant, this interaction would not be possible with the wild type PI3Kγ. The lack of an interaction at this position in the wild type PI3Kγ is consistent with the unimpaired binding of PI3Kγ to the Ras E37G mutant. The residue in PI3Kα at the position equivalent to PI3Kγ Val-223 is also a Val (Val-193). This suggests that in PI3Kα another residue in the vicinity of Val-193 interacts with Ras Glu-37. One candidate for such a residue is Lys-206 of PI3Kα. Neither PI3Kγ nor PI3Kδ have a basic residue at the equivalent position.

D38E is a Ras mutant that selectively activates the Raf signalling pathway. PI3Kα (Rodriguez-Viciana et al., 1997), PI3Kδ (Kinashi et al., 2000) and PI3Kγ (FIG. 2D) all fail to bind the Ras D38E mutant. Asp-38 makes important contacts with the RBDs of all three effectors and D38A prevents Ras activation of these pathways. Although the D38E mutation conserves the negative charge, it introduces a larger residue into the intricate interface. This is only tolerated in Raf because Thr-68 of Raf leaves sufficient space in the vicinity of the side chain of Ras residue 38. In PI3Kγ, the bulky Gln-231 occupies the space analogous to Thr-68 of Raf and prevents Ras D38E binding (data not shown), while in RalGDS, this space is filled by Lys-32. In PI3Kα and PI3Kδ a bulky residue (Tyr-207 and Phe-203, respectively) also occupies this space.

One Ras mutation that has been used widely to selectively activate PI3Kα is the Y40C mutation. Surprisingly, this mutation eliminates binding to PI3Kδ (Kinashi et al., 2000) and greatly attenuates binding to PI3Kγ (FIG. 2D). Tyr-40 forms a putative hydrogen bond to Gln-231. Mutation of Tyr-40 to Cys probably eliminates this potential hydrogen bond and reduces PI3Kγ binding. Since Gln-231 is not conserved in PI3Kα, the hydrogen bond between Ras Tyr-40 may not be present, and the Y40C mutation would not affect binding. In complexes with Raf and RalGDS, the role of Ras Tyr-40 appears to be to restrict the motion of a nearby basic residue in the RBD (Arg-89 in Raf and Lys-32 in RalGDS) which forms a critical salt bridge with Ras Asp-38.

The Ras mutant Y64G selectively inhibits PI3K and neurofibromin binding (Moodie et al., 1995). Ras Tyr-64 in switch II forms an apparent hydrogen bond to the side chain of Lys-234 and makes hydrophobic contacts with Phe-221 of PI3Kγ. These interactions would be lost in the Y64G mutant. Tyr-64 also stacks with the carboxylate of Ras Glu-63, which forms a hydrogen bond with Lys-234. In contrast, neither Raf nor RalGDS make any contact with any residue in switch II (Nassar et al., 1995; Vetter et al., 1999).

Conformational Change in the Ras.PI3Kγ Complex

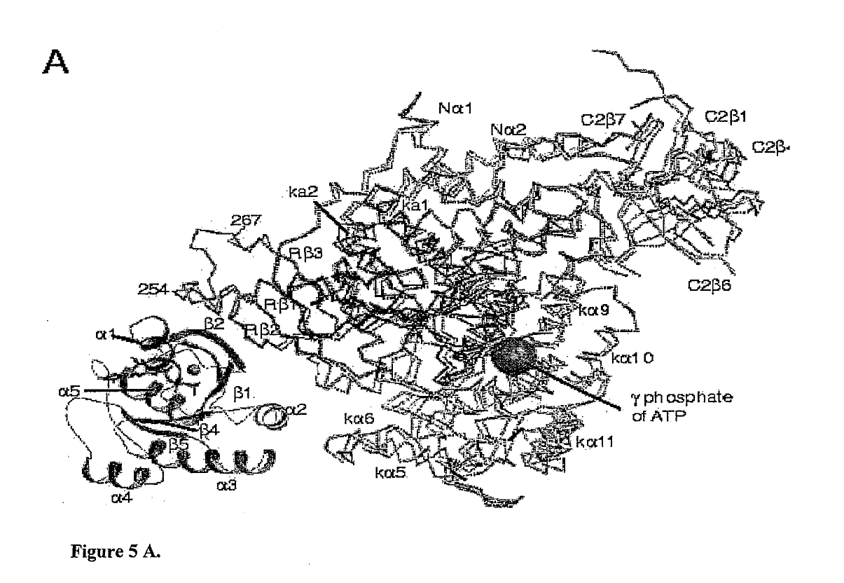

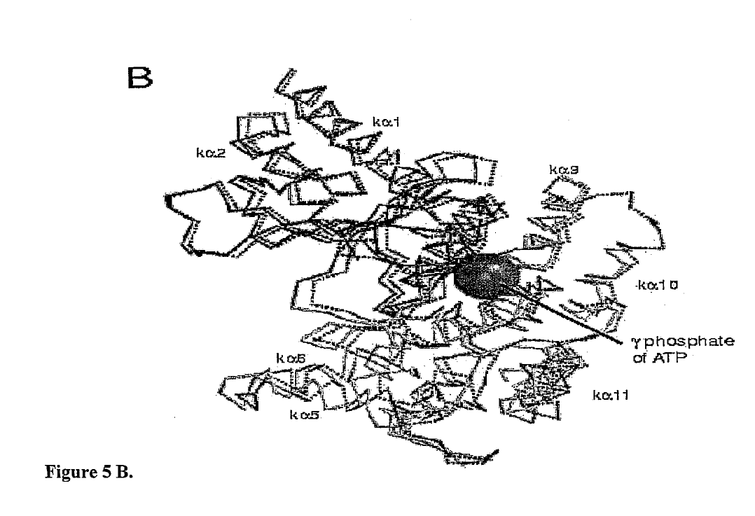

In addition to the induced fit of residues 255-267 at the interface with Ras, the structure of the Ras.PI3Kγ complex demonstrates that there are other more widespread conformational changes relative to the structure of the enzyme in the absence of Ras. Most of the conformational change, as illustrated in FIG. 5A, occurs in the C2 domain and the C-terminal lobe of the catalytic domain. When the structure of the free PB3Kγ and the structure of the complex are superimposed on the Ras-binding domain, the RMS deviation of the Cα atoms for the C2 domain is 1.7 Å. The RMS deviation of the Cα atoms for the C-terminal lobe of the catalytic domain is 1.9 Å. These values are well outside the cross-validated σ_{A} estimate of the error of the atom positions, which is 0.7 Å (based on the free set of reflections). Neither the N-terminal lobe of the catalytic domain nor the overall RBD show significant conformational change. The deviation of the helical domain in the structure of the complex from the free PI3Kγ structure is probably due to proteolysis in this domain (see methods).

In PI3Kγ, the ATP binding site is located between the N- and C-terminal lobes of the catalytic domain. As in the protein kinases (Zhou and Adams, 1997), mant-ADP release is slower than mant-ATP release (FIG. 2C). In the Ras.PI3Kγ complex, helix kα6 in the C-terminal lobe of the catalytic domain is pulled toward the Ras, and the entire ATP binding site moves with it, but apart from Asp-964 none of the residues in the ATP-binding site significantly change conformation. Consistent with this observation, neither the dissociation rate constants nor the K_{d} for either mant-ATP or mant-ADP change in the presence of Ras.

Unlike the ATP binding pocket, the putative phosphoinositide headgroup binding site appears to substantially change conformation upon Ras binding, as the C-terminal lobe of the catalytic domain pivots around the N-terminal lobe (FIG. 5B). Unfortunately, we have been unable to grow crystals in the presence of a phospholipid headgroup analogue, so we cannot be sure of the detailed structural changes relevant to headgroup binding. The backbones of helices kα9, kα10, and kα11, as well as kβ10 and kα7, which hold the activation loop in place, all substantially change position. Any allosteric mechanism of activation would probably involve a change in the affinity of phospholipid substrate binding, or perhaps an increase in the k_{cat} of the enzyme. Attempts to quantify binding of short chain phospholipid analogues by intrinsic tryptophan fluorescence were unsuccessful due to lack of significant change in the protein fluorescence.

The consistency of the biochemical and structural data suggests that this structure demonstrates a plausible mode of Ras-induced conformational changes in PI3Kγ.

A Model for Ras Activation of PI3K

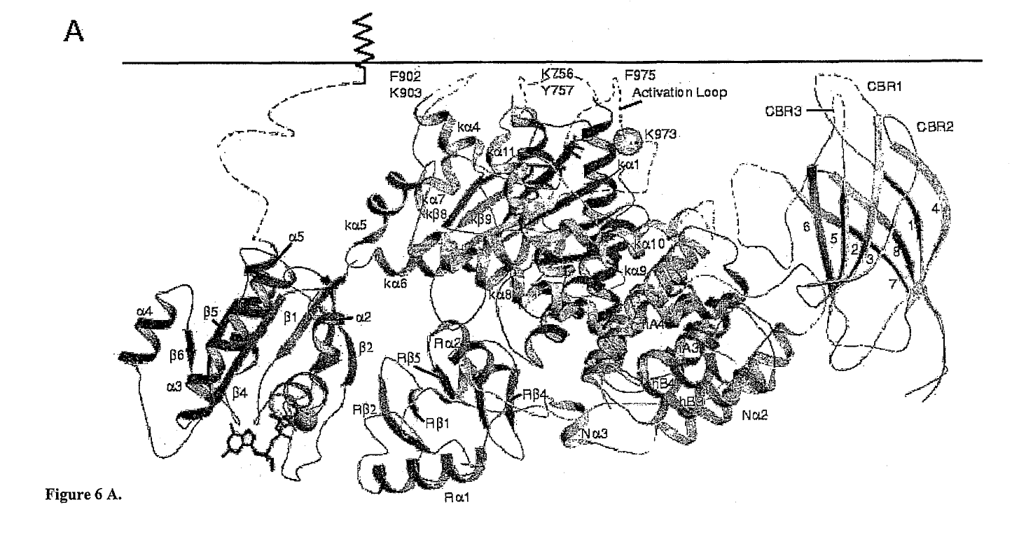

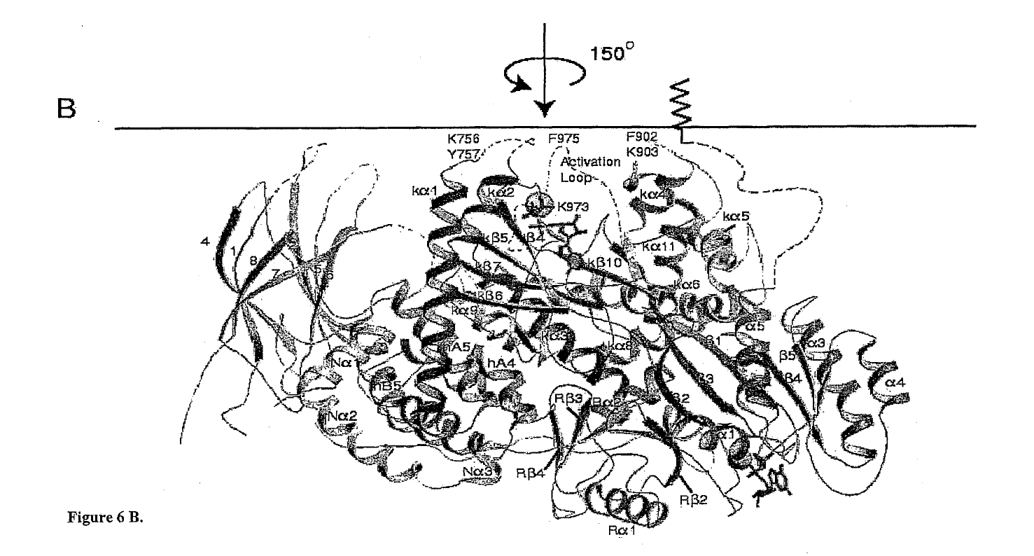

The structure of the Ras bound to PI3Kγ was used to construct a model of the activated complex on the plasma membrane. When the PI3Kγ active site is oriented towards the membrane surface and the C2 domain is binding phospholipids, the 20 residue tail of Ras (not present in the Ras construct used for crystallisation) has ample length to span the gap between the Ras effector domain and its farnesyl membrane anchor (FIG. 6). The loops of the PI3Kγ catalytic domain near the putative membrane interface all contain basic residues which could bind negatively charged phospholipid headgroups and hydrophobic residues capable of inserting into the membrane to help tether the enzyme in place.

In the Ras.PI3Kγ complex, the C2 and catalytic domains are spread apart, (FIG. 5A) presenting the active site to the membrane. This structural change may affect phosphoinositide binding in the active site, or increase the general affinity of the enzyme for the membrane interface. Although a substantial component of the Ras activation of PI3K appears to be via translocation to the membrane, an allosteric mechanism as suggested by the conformational change may also be a component of the Ras activation.

The synergistic stimulation of PI3Kγ by Gβγ and Ras is consistent with the emerging view of PI3Ks as integrators of multiple signalling pathways. The success of our design of both complex-stabilising and destabilising mutants, in conjunction with the novel interactions observed in the Ras.PI3Kγ structure, suggest new possibilities for the design of PI3K and Ras variants to control specific signal transduction pathways.

### Example 3

Flexible Membrane Anchors

As described above, peptide ligands may incorporate a flexible membrane anchor, such that they are flexibly tethered to the membrane surface, for example, CAAX fused to a Ras-binding domain. This Example shows that such a membrane anchor containing peptide inhibits Ras signaling to a greater degree than one without a membrane anchor.

An isolated Raf-1 Ras Binding Domain (RBD) is capable of blocking tumour cell growth in soft agar (Fridman et al., 1994). We describe here a novel modification of the isolated Raf-1 RBD that increases its ability to competitively inhibit Ras signalling and permits it to block signal propagation from specific Ras isotypes. We make chimeric proteins containing the Raf RBD fused to the hypervariable, 20 amino acid C-termini of H-, K-, and N-Ras. These Ras “tails” target the RBD to the plasma membrane, presumably increasing its effective concentration in the vicinity of the Ras isotype with the same tail.

We measure Ras signalling activity by using the PathDetect system (Stratagene). We transfecte CHO cells, which have low endogenous levels of Ras (Choy et al., 1999), with 150 ng of a reporter plasmid (pFR-Luc) containing a Photinus pyralis (firefly) luciferase gene driven by a GAL4 promoter, and 10 ng of a plasmid (pFA2-Elk1-dbd) containing a GAL4 dbd-Elk1 activation domain fusion protein behind a CMV promoter. Activation of the MAP kinase signalling cascade leads to phosporylation of the Elk1 domain, dimerization of the GAL4-Elk-1 fusion protein, and transcription of Photinus luciferase. To control for transfection efficiency and levels of expression, we also transfect 10 ng of a reporter control plasmid (pRL-CMV) containing the gene for Renilla reniformis (sea pansy) luciferase behind a CMV promoter, and measure the luminescence generated by both Photinus and Renilla luciferase using the DualLuciferase assay system (Promega). All luciferase data are expressed as the ratio of Photinus luminescence to Renilla luminescence. When indicated, cells are transfected with 20 ng of pcDNA3 containing an RBD-Ras tail chimera protein and 1 ng of H-Ras G12V in an EXV vector. The total amount of transfected DNA is kept constant at 210 ng by addition of empty pcDNA3 vector.

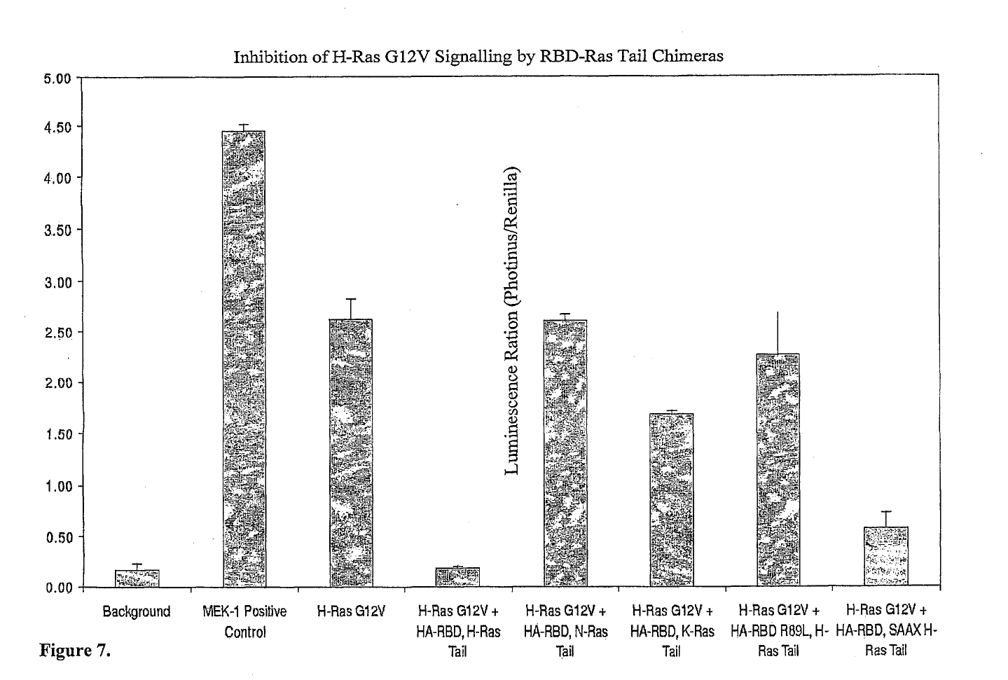

The results are shown in FIG. 7 below.

CHO cells transfected with the reporter system plasmids (pFR-Luc, pFA2-Elk1-dbd, and pRL-CMV) exhibit relatively low ratios of Photinus to Renilla luciferase activity (0.17±0.06). Transfection of H-Ras G12V raises this ratio substantially, to 2.6±0.2. Addition of Raf-1 RBD with an H-Ras tail reduces the luciferase ratio to background levels (0.18±0.02). This represents a 93% inhibition of Ras signalling activity via the MAP kinase cascade. Mutation of the cysteine in the CAAX box to a serine (SAAX) reduces the effectiveness of the inhibitor 3 fold, as represented by a luciferase ratio of 0.57±0.14. The R89L mutation in the Raf RBD, which eliminates binding of the Raf RBD to Ras as previously described (Block et al., 1996), does not block Ras simulation of luciferase production, as the ratio is 2.26±0.53. Interestingly, Raf RBD with a K-Ras tail only reduces the luciferase ratio by 36%, to 1.68±0.03, and Raf RBD with an N-Ras tail does not inhibit H-Ras G12V stimulation of the luciferase ratio. Positive and negative control transfections with pFC-MEK1 (containing MEK-1 behind a CMV promoter) and pFC2-dbd (containing the Gal4-DBD without the Elk1 fusion) indicate that the reporter system was functioning properly.

From these data we conclude that the H-Ras tail is capable of targeting the Raf-1 RBD to the regions of the plasma membrane where H-Ras is active, increasing the effective concentration of the Raf-1 RBD and its ability to competitively inhibit H-Ras signal transduction. Chimeras containing the Raf-1 RBD fused to K- or N-Ras tails do not inhibit R-Ras signalling effectively. Therefore, RBD-Ras tail chimeras function as isotype-specific inhibitors of Ras signalling. Isotype-specific inhibitors of Ras signalling are useful for determining the biological functions of the Ras isotypes. Furthermore, these more effective RBD-based inhibitors are able to specifically inhibit the Ras proteins in diseases such as cancer where they are improperly activated.

Reference is made to the following publications: Fridman, M., Tikoo, A., Varga, M., Murphy, A., Nur, E. K. M. S., and Maruta, H. (1994). The minimal fragments of c-Raf-1 and NF1 that can suppress v-Ha-Ras-induced malignant phenotype. J Biol. Chem. 269, 30105-30108; Choy, E., Chiu, V. K., Silletti, J., Feoktistov, M., Morimoto, T., Michaelson, D., Ivanov, I. E., and Philips, M. R. (1999). Endomembrane trafficking of ras: the CAAX motif targets proteins to the ER and Golgi. Cell 98, 69-80; and Block, C., Janknecht, R., Herrmann, C., Nassar, N., and Wittinghofer, A. (1996). Quantitative structure-activity analysis correlating Ras/Raf interaction in vitro to Raf activation in vivo. Nat. Struct. Biol. 3, 244-251.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or biology or related fields are intended to be covered by the present invention. All publications mentioned in the above specification are herein incorporated by reference.

References

Barnett, S. F., Ledder, L. M., Stirdivant, S. M., Ahern, J., Conroy, R, R., and Heimbrook, D. C. (1995). Interfacial catalysis by phosphoinositide 3′-hydroxykinase, Biochemistry 34, 14254-14262.

Bondeva, T., Pirola, L., Bulgarelli-Leva, G., Rubio, I., Wetzker, R., and Wymann, M. P. (1998). Bifurcation of lipid and protein kinase signals of PI3Kγ to the protein kinases PKB and MAPK, Science 282, 293-6.

Borlack-Sjodin, P. A., Margarit, S. M., Bar-Sagi, D., and Kuriyan, J. (1998). The structural basis of the activation of Ras by Sos, Nature 394, 337-343.

Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., et al. (1998). Crystallography & NMR system: A new software suite for macromolecular structure determination, Acta Crystallogr D Biol Crystalogr 54, 905-921.

Carpenter, C., Auger, K., Chanudhuri, M., Yoakim, M., Schaffhausen, B., Shoelson, S., and Cantley, L. (1993). Phosphoinositide 3-kinase is activated by phosphopeptides that bind to the SH2 domains of the 85-kDa subunit, J Biol Chem 268, 9478-9483.

CCP4 (1994). Collaborative Computing Project 4: A suite of programs for protein crystallography, Acta Crystallogr D 50, 760-763.

Coffer, P. J., Geijsen, N., M'Rabet, L., Schweizer, R. C., Maikoe, T., Raaijmakers, J. A., Lammers, J. W., and Koenderman, L. (1998). Comparison of the roles of mitogen-activated protein kinase kinase and phosphatidylinositol 3-kinase signal transduction in neutrophil effector function, Biochem J 329, 121-130.

Condliffe, A. M., Hawkins, P. T., Stephens, L. R., Haslett, C., and Chilvers, E. R. (1998). Priming of human neutrophil superoxide generation by tumour necrosis factor-alpha is signalled by enhanced phosphatidylinositol 3,4,5-trisphosphate but not inositol 1,4,5-trisphosphate accumulation, FEBS Lett 439, 147-151.

Deora, A. A., Win, T., Vanhaesebroeck, B., and Lander, H. M. (1998). A redox-triggered ras-effector interaction. Recruitment of phosphatidylinositol 3′-kinase to Ras by redox stress, J Biol Chem 273, 29923-29928.

Domin, J., and Waterfield, M. D. (1997). Using structure to define the function of phosphoinositide 3-kinase family members, FEBS Lett 410, 91-95.

Engh, R. A., and Huber, R. (1991). Accurate bond and angle parameters for x-ray protein structure refinement, Acta Crystallogr A 47, 392-400.

Feig, L. A., Urano, T., and Cantor, S. (1996). Evidence for a Ras/Ral signalling cascade, Trends Biochem Sci 21, 438-441.

Franke, T. F., Kaplan, D. R., and Cantley, L. C. (1997). PI3K: downstream AKTion blocks apoptosis, Cell 88,435-437.

Gire, V., Marshall, C., and Wynford-Thomas, D. (2000). PI-3-kinase is an essential anti-apoptotic effector in the proliferative response of primary human epithelial cells to mutant RAS, Oncogene 19, 2269-2276.

Herrmann, C., Horn, G., Spaargaren, M., and Wittinghofer, A. (1996). Differential interaction of the ras family GTP-binding proteins H-Ras, Rap1A, and R-Ras with the putative effector molecules Raf kinase and Ral-guanine nucleotide exchange factor, J Biol Chem 271, 6794-6800.

Herrmann, C., Martin, G. A., and Wittinghofer, A. (1995). Quantitative analysis of the complex between p21ras and the Ras-binding domain of the human Raf-1 protein Idnase, J Biol Chem 270, 2901-2905.

Hiratsuka, T. (1983). New ribose-modified fluorescent analogues of adenine and guanine nucleotides available as substrates for various enzymes, Biochim Biophys Acta 742, 496-508.

Hirsch, B., Katanaev, V. L., Garlanda, C., Azzolino, O., Pirola, L., Silengo, L., Sozzani, S., Mantovani, A., Altruda, F., and Wymann, M. P. (2000). Central role for G protein-coupled phosphoinositide 3-kinase γ in inflammation, Science 287, 1049-1053.

Huang, L., Hofer, F., Martin, G. S., and Kim, S. -H. (1998). Structural Basis for the Interaction of Ras with RalGDS, Nat Struct Biol 5, 422-426.

Jameson, D. M., and Eccleston, J. F. (1997). Fluorescent nucleotide analogues: synthesis and applications, Methods Enzymol 278, 363-390.

Jones, T. A., Zou, J. Y., Cowan, S. W., and Kjeldgaard (1991). Improved methods for binding protein models in electron density maps and the location of errors in these models, Acta Crystallogr A 47, 110-119.

Kauffmann-Zeh, A., Rodriguez-Viciana, P., Ulrich, E., Gilbert, C., Coffer, P., Downward, J., and Evan, G. (1997). Suppression of c-Myc-induced apoptosis by Ras signalling through PI(3)K and PKB, Nature 385, 544-548.

Khwaja, A., Rodriguez-Viciana, P., Wentstrom, S., Warne, P. H., and Downward, J. (1997). Matrix adhesion and Ras transformation both activate a phosphoinositide 3-OH kinase and protein kinase B/Akt cellular survival pathway, Embo J 16,2783-2793.

Kinashi, T., Katagiri, K., Watanabe, S. -i., Vanhaesebroeck, B., Downward, J., and Takatsu, K. (2000). Distinct mechanisms of α5β1 integrin activation by H-Ras and R-Ras, J Biol Chem 275, 22590-22596.

Kraulis, P. J. (1991). MOLSCRIPT: A program to produce both detailed and schematic plots of protein structures., J Appl Crystallogr 24, 946-950.

Krugmann, S., Hawkins, P. T., Pryer, N., and Braselmann, S. (1999). Characterizing the interactions between the two subunits of the p101/p110γ phosphoinositide 3-kinase and their role in the activation of this enzyme by Gβγ subunits, J Biol Chem 274, 17152-17158.

Kurosu, H., Maehama, T., Okada, T., Yamamoto, T., Hoshino, S., Fukui, Y., Ui, M., Hazeki, O., and Katada, T. (1997). Heterodimeric phosphoinositide 3-kinase consisting of p85 and p110beta is synergistically activated by tie betagamma subunits of G proteins and phosphotyrosyl peptide, J Biol Chem 272, 24252-24256.

Leslie, A. G. W. (1992). Recent changes to the MOSFLM package for film and image plate data. In Joint CCP4 and ESF-EACMB Newsletter on Protein Crystallography (Warrington, UK, Daresbury Laboratory).

Li, Z., Jiang, H., Xie, W., Zhang, Z., Smrka, A. V., and Wu, D. (2000). Roles of PLC-β2 and -β3 and PI3Kγ in Chemoattractant-Mediated Signal Transduction, Science 287, 1046-1049.

Lowy, D. R., and Willumsen, B. M. (1993). Function and regulation of Ras, Annu Rev Biochem 62, 851-891.

Maier, U., Babich, A., and Nurnberg, B. (1999). Roles of non-catalytic subunits in Gβγ-induced activation of class I phosphoinositide 3-kinase isoforms β and γ, J Biol Chem 274, 29311-29317.

Marshall, C. J. (1996). Ras effectors, Curr Opin Cell Biol 8, 197-204.

Merritt, E. A., and Bacon, D. J. (1997). Raster3D: Photorealistic Molecular Graphics, Meth Enzymol 277, 505-524.

Milburn, M. V., Tong, L., deVos, A. M., Brunger, A., Yamaizumi, Z., Nishimura, S., and Kim, S. H. (1990). Molecular switch for signal transduction: structural differences between active and inactive forms of protooncogenic ras proteins, Science 247, 939-945.

Moodie, S. A., Paris, M., Villafranca, E., Kirshmeier, P., Willumsen, B. M., and Wolfman, A. (1995). Different structural requirements within the switch II region of the Ras protein for interactions with specific downstream targets, Oncogene 11, 447-454.

Moore, K. J., Webb, M. R., and Eccleston, J. F. (1993). Mechanism of GTP hydrolysis by p21N-ras catalyzed by GAP: studies with a fluorescent GTP analogue, Biochemistry 32, 7451-7459.

Murga, C., Fukuhara, S., and Gutkind, J. S. (2000). A novel role for phosphatidylinositol 3-kinase β in signalling from G protein-coupled receptors to Akt, J Biol Chem 275, 12069-12073.

Nassar, N., Horn, G., Herrmann, C., Scherer, A., McCormick, P., and Wittinghofer, A. (1995). The 2.2 A crystal structure of the Ras-binding domain of the serine/threonine kinase c-Raft in complex with Rap1A and a GTP analogue, Nature 375, 554-560.

Nicholls, A., Sharp, K. A., and Honig, B. (1991). Protein folding and association: insights from the interfacial and thermodynamic properties of hydrocarbons, Proteins Struct Funct Genet 11, 281-296.

Pai, E. F., Kabsch, W., Krengel, U., Holmes, K. C., John, J., and Wittinghofer, A. (1989). Structure of the guanine-nucleotide-binding domain of the Ha-ras oncogene product p21 in the triphosphate conformation, Nature 341, 209-214.

Pai, E. F., Krengel, U., Petsko, G. A., Goody, R. S., Kabsch, W., and Wittinghofer, A. (1990). Refined crystal structure of the triphosphate conformation of H-ras p21 at 1.35 A resolution: implications for the mechanism of GTP hydrolysis, Embo J 9, 2351-2359.

Polakis, P., and McCormick, F. (1993). Structural requirements for the interaction of p21ras with GAP, exchange factors, and its biological effector target, J Biol Chem 268, 9157-9160.

Rodriguez-Viciana, P., Marte, B. M., Warne, P. H., and Downward, J. (1996a). Phosphatidylinositol 3′ kinase: one of the effectors of Ras, Philos Trans R Soc Lond B Biol Sci 351, 225-232.

Rodriguez-Viciana, P., Warne, P. H., Dhand, R., Vanhaesebroeck, B., Gout, I., Fry, M. J., Waterfield, M. D., and Downward, J. (1994). Phosphatidylinositol-3-OH kinase as a direct target of Ras, Nature 370, 527-532.

Rodriguez-Viciana, P., Warne, P. H., Khwaja, A., Marte, B. M., Pappin, D., Das, P., Waterfield, M. D., Ridley, A., and Downward, J. (1997). Role of phosphoinositide 3-OH kinase in cell transformation and control of the actin cytoskeleton by Ras, Cell 89, 457-467.

Rodriguez-Viciana, P., Warne, P. H., Vanhaesebroeck, B., Waterfield, M. D., and Downward, J. (1996b). Activation of phosphoinositide 3-kinase by interaction with Ras and by point mutation, Embo J 15, 2442-2451.

Rubio, I., Rodriguez-Viciana, P., Downward, I., and Wetzker, R. (1997). Interaction of Ras with phosphoinositide 3-kinase γ, Biochem J 326, 891-895.

Rubio, I., Wittig, U., Meyer, C., Heinze, R., Kadereit, D., Waldmann, H., Downward, J., and Wetzker, R. (1999). Farnesylation of Ras is important for the interaction with phosphoinositide 3-kinase γ, Eur J Biochem 266, 70-82.

Runnels, L. W., and Scarlata, S. F. (1998). Regulation of the rate and extent of phospholipase Cβ2 effector activation by the βγ subunits of heterotrimeric G proteins, Biochemistry 37, 15563-15574.

Sasaki, T., Irie-Sasaki, J., Jones, R. G., Oliveira-dos-Santos, A. J., Stanford, W. L., Bolon, B., Wakeham, A., Itie, A., Bouchard, D., Kozieradzkd, I., et al. (2000). Function of PI3Kγ in thymocyte development, T cell activation, and neutrophil migration, Science 287, 1040-1046.

Scheffzek K., Ahmadian, M. R., Kabsch, W., Wiesmuller, L., Lautwein, A., Schmitz, F., and Wittinghofer, A. (1997). The Ras-RasGAP complex: Structural basis for GTPase activation and its loss in oncogenic Ras mutants, Science 277, 333-338.

Shepherd, P. R., Withers, D. J., and Siddle, K. (1998). Phosphoinositide 3-kinase: the key switch mechanism in insulin signalling, Biochem J 333, 471-490.

Shields, J. M., Pruitt, K., McFall, A., Shaub, A., and Der, C. J. (2000). Understanding Ras: ‘it ain't over 'til it's over’, Trends Cell Biol 10, 147-154.

Shioi, T., Kang, P. M., Douglas, P. S., Hampe, J., Yballe, C. M., Lawitts, J., Cantley, L. C., and Izumo, S. (2000). The conserved phosphoinositide 3-kinase pathway determines heart size in mice, Embo J 19, 2537-2548.

Stephens, L. R., Eguinoa, A., Erdjument-Bromage, H., Lui, M., Cooke, F., Coadwell, J., Smrcka, A. S., Thelen, M., Cadwallader, K., Tempst, P., and Hawkins, P. T. (1997). The Gay sensitivity of a PI3K is dependent upon a tightly associated adaptor, p101, Cell 89, 105-114.

Stephens, L. R., Hughes, K. T., and Ivine, R. F. (1991). Pathway of phosphatidylinositol(3,4,5)-trisphosphate synthesis in activated neutrophils, Nature 351, 33-39.

Stoyanov, B., Volinia, S., Hanck, T., Rubio, I., Loubtchenkov, M., Malek, D., Stoyanova, S., Vanhaesebroeck, B., Dhand, R., Nurnberg, B., et al. (1995). Cloning and Characterization of a G Protein-Activated Human Phosphoinositide 3-Kinase, Science 269, 690-693.

Sydor, J. R., Engelhard, M., Wittinghofer, A., Goody, R. S., and Herrmann, C. (1998). Transient kinetic studies on the interaction of Ras and the Ras-binding domain of c-Raf-1 reveal rapid equilibration of the complex, Biochemistry 37, 14292-14299.

Tanaka, Y., Minami, Y., Mine, S., Hirano, H., Hu, C. D., Fujimoto, H., Fujii, K., Saito, K., Tsukada, J., van Kooyk, Y., et al. (1999). H-Ras signals to cytoskeletal machinery in induction of integrin-mediated adhesion of T cells, J Immunol 163, 6209-6216.

Toker, A., and Cantley, L. C. (1997). Signalling through the lipid products of phosphoinositide-3-OH kinase, Nature 387, 673-676.

Vanhaesebroeck, B., and Waterfield, M. D. (1999). Signaling by distinct classes of phosphoinositide 3-kinases, Exp Cell Res 253, 239-254.

Vanhaesebroeck, B., Welham, M. J., Kotani, K., Stein, R., Warne, P. H, Zvelebil, M. J., Higashi, K., Volinia, S., Downward, J., and Waterfield, M. D. (1997). P110δ, a novel phosphoinositide 3-kinase in leukocytes, Proc Natl Acad Sci U S A 94, 4330-4335.

Vetter, I. R., Linnemann, T., Wohlgemuth, S., Geyer, M., Kalbitzer, H. R, Herrmann, C., and Wittinghofer, A. (1999). Structural and biochemical analysis of Ras-effector signalling via RalGDS, FEBS Lett 451, 175-180.

Walker, E. H., Perisic, O., Ried, C., Stephens, L., and Williams, R. L. (1999). Structural insights into phosphoinositide 3-kinase catalysis and signalling, Nature 402, 313-320.

White, M. A., Nicolette, C., Minden, A., Polverino, A., Van Aelst, L., Karin, M., and Wigler, M. H. (1995). Multiple Ras functions can contribute to mammalian cell transformation, Cell 80, 533-541.

Wittinghofer, A., and Herrmann, C. (1995). Ras-effector interactions, the problem of specificity, FEBS Lett 369, 52-56.

Worthen, G. S., Avdi N., Buhl, A. M., Suzuki, N., and Johnson, G. L. (1994). FMLP activates Ras and Raf in human neutrophils. Potential role in activation of MAP kinase, J Clin Invest 94, 815-823.

Wymann, M. P., Sozzani, S., Altruda, F., Mantovani, A., and Hirsch, E. (2000). Lipids on the move: phosphoinositide 3-kinases in leukocyte function, Immunology Today 21, 260-264.

Xue, L., Murray, J. H., and Tolkovsky, A. M. (2000). The Ras/phosphatidylinositol 3-kinase and Ras/ERK pathways function as independent survival modules each of which inhibits a distinct apoptotic signalling pathway in sympathetic neurons, J Biol Chem 275, 8817-8924.

Zhou, J., and Adams, J. A. (1997). Participation of ADP dissociation in the rate-determining step in cAMP-dependent protein kinase, Biochemistry 36, 15733-15738.

TABLE 1
Data collection, structure determination and refinement statistics

| | Resolution | Observations/ | Completeness (last shell) | | |
|---|---|---|---|---|---|
| Data set³ | (Å) | unique reflections | (%) | R_{sym}⁴ | <I/σ> (last shell) |
| Crystal 1 | 50-3.2 | 146371/23318 | 100.0 (100.0) | 0.112 | 21.8 (3.6) |
| Crystal 2¹ | 50-3.0 | 270093/24791 | 82.4 (51.3) | 0.094 | 14.7 (1.7) |
| Merge² | 50.0-3.0 | 232825/25822 | 91.6 (50.5) | 0.102 | 20.0 (1.7) |

Refinement statistics

| | Resolution | | | Reflections | | | R_{free}⁵ | R.m.s.d. from ideality⁶ | | |
|---|---|---|---|---|---|---|---|---|---|---|
| Data set | (Å) | Protein atoms | Waters | Work | Free | R_{cryst}⁵ | (% data used) | Bonds | Angles | Dihedrals |
| Merge | 50.0-3.0 | 7405 | 6 | 24273 | 1510 | 0.213 | 0.282 (5.4) | 0.0093 Å | 1.29° | 22.7° |

¹Only 50 degrees were collected due to radiation damage caused by long exposure.
²The merged dataset was created by combining the data from crystals 1 and 2 in SCALA.
³The data from crystals 1 and 2 were collected at ESRF Beamline ID14-1.
⁴R_{sym} = ΣhklΣi|Iᵢ(hkl) − <I(hkl)>|/ΣhklΣi Iᵢ(hkl)
⁵R_{cryst} and R_{free} = Σ|F_{obs} − F_{calc}|/ΣF_{obs}; R_{free} calculated with the percentage of the data shown in parentheses
⁶R.m.s deviations for bond angles and lengths in regard to Engh & Huber parameters (Engh and Huber, 1991)

REMARK Written by O version 7.0.0
REMARK Fri Jun 2 20:32:07 2000

| | | | | | | |
|---|---|---|---|---|---|---|
| CRYST1 | 113.635 | 113.635 | 183.881 | 90.00 | 90.00 | 120.00 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.008800 | 0.005080 | 0.000000 | 0.00000 |
| SCALE2 | 0.000000 | 0.010161 | 0.000000 | 0.00000 |
| SCALE3 | 0.000000 | 0.000000 | 0.005438 | 0.00000 |

| | | | | | | | | | | | |
|---|---|---|---|---|---|---|---|---|---|---|---|
| ATOM | 1 | CB | ALA | A | 143 | 76.520 | 95.646 | 61.236 | 1.00 | 71.64 | 6 |
| ATOM | 2 | C | ALA | A | 143 | 76.296 | 97.493 | 62.914 | 1.00 | 71.01 | 6 |
| ATOM | 3 | O | ALA | A | 143 | 76.857 | 98.203 | 63.747 | 1.00 | 70.81 | 8 |
| ATOM | 4 | N | ALA | A | 143 | 78.505 | 96.948 | 61.921 | 1.00 | 71.67 | 7 |
| ATOM | 5 | CA | ALA | A | 143 | 77.037 | 97.016 | 61.667 | 1.00 | 71.57 | 6 |
| ATOM | 6 | N | SER | A | 144 | 75.033 | 97.096 | 63.036 | 1.00 | 70.60 | 7 |
| ATOM | 7 | CA | SER | A | 144 | 74.215 | 97.487 | 64.177 | 1.00 | 69.78 | 6 |
| ATOM | 8 | CB | SER | A | 144 | 72.792 | 96.942 | 64.017 | 1.00 | 70.14 | 6 |
| ATOM | 9 | OG | SER | A | 144 | 72.106 | 97.581 | 62.956 | 1.00 | 70.82 | 8 |
| ATOM | 10 | C | SER | A | 144 | 74.792 | 96.997 | 65.499 | 1.00 | 69.28 | 6 |
| ATOM | 11 | O | SER | A | 144 | 75.759 | 96.236 | 65.529 | 1.00 | 69.54 | 8 |
| ATOM | 12 | N | GLU | A | 145 | 74.190 | 97.447 | 66.595 | 1.00 | 68.35 | 7 |
| ATOM | 13 | CA | GLU | A | 145 | 74.620 | 97.041 | 67.924 | 1.00 | 67.09 | 6 |
| ATOM | 14 | CB | GLU | A | 145 | 73.886 | 97.865 | 68.985 | 1.00 | 68.59 | 6 |
| ATOM | 15 | CG | GLU | A | 145 | 74.608 | 97.968 | 70.326 | 1.00 | 70.94 | 6 |
| ATOM | 16 | CD | GLU | A | 145 | 74.801 | 96.625 | 71.002 | 1.00 | 71.79 | 6 |
| ATOM | 17 | OE1 | GLU | A | 145 | 73.790 | 96.009 | 71.403 | 1.00 | 71.80 | 8 |
| ATOM | 18 | OE2 | GLU | A | 145 | 75.966 | 96.186 | 71.129 | 1.00 | 72.30 | 8 |
| ATOM | 19 | C | GLU | A | 145 | 74.238 | 95.566 | 68.034 | 1.00 | 65.70 | 6 |
| ATOM | 20 | O | GLU | A | 145 | 74.774 | 94.825 | 68.857 | 1.00 | 64.95 | 8 |
| ATOM | 21 | N | GLU | A | 146 | 73.307 | 95.156 | 67.177 | 1.00 | 63.78 | 7 |
| ATOM | 22 | CA | GLU | A | 146 | 72.823 | 93.782 | 67.121 | 1.00 | 62.92 | 6 |
| ATOM | 23 | CB | GLU | A | 146 | 71.735 | 93.666 | 66.050 | 1.00 | 64.45 | 6 |
| ATOM | 24 | CG | GLU | A | 146 | 70.352 | 93.295 | 66.564 | 1.00 | 67.31 | 6 |
| ATOM | 25 | CD | GLU | A | 146 | 70.227 | 91.823 | 66.931 | 1.00 | 69.16 | 6 |
| ATOM | 26 | OE1 | GLU | A | 146 | 70.570 | 90.967 | 66.085 | 1.00 | 69.60 | 8 |
| ATOM | 27 | OE2 | GLU | A | 146 | 69.777 | 91.522 | 68.058 | 1.00 | 70.04 | 8 |
| ATOM | 28 | C | GLU | A | 146 | 73.964 | 92.827 | 66.785 | 1.00 | 61.15 | 6 |
| ATOM | 29 | O | GLU | A | 146 | 74.273 | 91.916 | 67.555 | 1.00 | 60.60 | 8 |
| ATOM | 30 | N | SER | A | 147 | 74.583 | 93.049 | 65.627 | 1.00 | 59.35 | 7 |
| ATOM | 31 | CA | SER | A | 147 | 75.686 | 92.221 | 65.153 | 1.00 | 57.82 | 6 |
| ATOM | 32 | CB | SER | A | 147 | 76.178 | 92.725 | 63.794 | 1.00 | 58.77 | 6 |
| ATOM | 33 | OG | SER | A | 147 | 76.730 | 94.023 | 63.902 | 1.00 | 60.62 | 8 |
| ATOM | 34 | C | SER | A | 147 | 76.850 | 92.198 | 66.136 | 1.00 | 55.79 | 6 |
| ATOM | 35 | O | SER | A | 147 | 77.498 | 91.169 | 66.314 | 1.00 | 54.64 | 8 |
| ATOM | 36 | N | GLN | A | 148 | 77.117 | 93.339 | 66.760 | 1.00 | 53.66 | 7 |
| ATOM | 37 | CA | GLN | A | 148 | 78.197 | 93.431 | 67.730 | 1.00 | 52.68 | 6 |
| ATOM | 38 | CB | GLN | A | 148 | 78.389 | 94.886 | 68.167 | 1.00 | 54.73 | 6 |
| ATOM | 39 | CG | GLN | A | 148 | 79.100 | 95.734 | 67.123 | 1.00 | 57.72 | 6 |
| ATOM | 40 | CD | GLN | A | 148 | 78.605 | 97.167 | 67.090 | 1.00 | 59.75 | 6 |
| ATOM | 41 | OE1 | GLN | A | 148 | 78.633 | 97.873 | 68.099 | 1.00 | 60.43 | 8 |
| ATOM | 42 | NE2 | GLN | A | 148 | 78.150 | 97.605 | 65.922 | 1.00 | 60.03 | 7 |
| ATOM | 43 | C | GLN | A | 148 | 77.886 | 92.553 | 68.933 | 1.00 | 50.70 | 6 |
| ATOM | 44 | O | GLN | A | 148 | 78.740 | 91.789 | 69.390 | 1.00 | 50.82 | 8 |
| ATOM | 45 | N | ALA | A | 149 | 76.661 | 92.659 | 69.440 | 1.00 | 47.06 | 7 |
| ATOM | 46 | CA | ALA | A | 149 | 76.237 | 91.862 | 70.584 | 1.00 | 44.11 | 6 |
| ATOM | 47 | CB | ALA | A | 149 | 74.816 | 92.231 | 70.982 | 1.00 | 43.91 | 6 |
| ATOM | 48 | C | ALA | A | 149 | 76.311 | 90.380 | 70.225 | 1.00 | 41.90 | 6 |
| ATOM | 49 | O | ALA | A | 149 | 76.597 | 89.542 | 71.077 | 1.00 | 40.82 | 8 |
| ATOM | 50 | N | PHE | A | 150 | 76.046 | 90.069 | 68.961 | 1.00 | 39.57 | 7 |
| ATOM | 51 | CA | PHE | A | 150 | 76.094 | 88.689 | 68.484 | 1.00 | 39.29 | 6 |
| ATOM | 52 | CB | PHE | A | 150 | 75.490 | 88.604 | 67.076 | 1.00 | 38.89 | 6 |
| ATOM | 53 | CG | PHE | A | 150 | 75.454 | 87.210 | 66.503 | 1.00 | 40.47 | 6 |
| ATOM | 54 | CD1 | PHE | A | 150 | 74.739 | 86.198 | 67.136 | 1.00 | 39.24 | 6 |
| ATOM | 55 | CD2 | PHE | A | 150 | 76.134 | 86.910 | 65.324 | 1.00 | 38.01 | 6 |
| ATOM | 56 | CE1 | PHE | A | 150 | 74.703 | 84.910 | 66.603 | 1.00 | 39.21 | 6 |
| ATOM | 57 | CE2 | PHE | A | 150 | 76.102 | 85.621 | 64.784 | 1.00 | 38.46 | 6 |
| ATOM | 58 | CZ | PHE | A | 150 | 75.387 | 84.624 | 65.425 | 1.00 | 37.98 | 6 |
| ATOM | 59 | C | PHE | A | 150 | 77.545 | 88.189 | 68.479 | 1.00 | 38.70 | 6 |
| ATOM | 60 | O | PHE | A | 150 | 77.841 | 87.125 | 69.017 | 1.00 | 36.79 | 8 |
| ATOM | 61 | N | GLN | A | 151 | 78.450 | 88.962 | 67.878 | 1.00 | 38.39 | 7 |
| ATOM | 62 | CA | GLN | A | 151 | 79.853 | 88.568 | 67.839 | 1.00 | 38.19 | 6 |
| ATOM | 63 | CB | GLN | A | 151 | 80.700 | 89.645 | 67.154 | 1.00 | 39.36 | 6 |
| ATOM | 64 | CG | GLN | A | 151 | 82.142 | 89.215 | 66.846 | 1.00 | 40.06 | 6 |
| ATOM | 65 | CD | GLN | A | 151 | 82.219 | 88.004 | 65.917 | 1.00 | 41.30 | 6 |
| ATOM | 66 | OE1 | GLN | A | 151 | 81.747 | 88.043 | 64.779 | 1.00 | 40.98 | 8 |
| ATOM | 67 | NE2 | GLN | A | 151 | 82.816 | 86.924 | 66.404 | 1.00 | 40.56 | 7 |
| ATOM | 68 | C | GLN | A | 151 | 80.307 | 88.370 | 69.281 | 1.00 | 38.33 | 6 |
| ATOM | 69 | O | GLN | A | 151 | 81.091 | 87.474 | 69.580 | 1.00 | 36.49 | 8 |
| ATOM | 70 | N | ARG | A | 152 | 79.792 | 89.210 | 70.173 | 1.00 | 38.32 | 7 |
| ATOM | 71 | CA | ARG | A | 152 | 80.107 | 89.125 | 71.591 | 1.00 | 39.98 | 6 |
| ATOM | 72 | CB | ARG | A | 152 | 79.411 | 90.269 | 72.346 | 1.00 | 44.06 | 6 |
| ATOM | 73 | CG | ARG | A | 152 | 79.327 | 90.103 | 73.862 | 1.00 | 48.48 | 6 |
| ATOM | 74 | CD | ARG | A | 152 | 78.764 | 91.371 | 74.532 | 1.00 | 54.07 | 6 |
| ATOM | 75 | NE | ARG | A | 152 | 78.504 | 91.182 | 75.963 | 1.00 | 56.58 | 7 |
| ATOM | 76 | CZ | ARG | A | 152 | 78.154 | 92.152 | 76.806 | 1.00 | 57.98 | 6 |
| ATOM | 77 | NH1 | ARG | A | 152 | 78.017 | 93.401 | 76.378 | 1.00 | 57.77 | 7 |
| ATOM | 78 | NH2 | ARG | A | 152 | 77.937 | 91.873 | 78.088 | 1.00 | 57.84 | 7 |
| ATOM | 79 | C | ARG | A | 152 | 79.668 | 87.772 | 72.153 | 1.00 | 39.21 | 6 |
| ATOM | 80 | O | ARG | A | 152 | 80.373 | 87.174 | 72.965 | 1.00 | 37.67 | 8 |
| ATOM | 81 | N | GLN | A | 153 | 78.502 | 87.291 | 71.725 | 1.00 | 37.26 | 7 |
| ATOM | 82 | CA | GLN | A | 153 | 78.008 | 86.004 | 72.204 | 1.00 | 37.34 | 6 |
| ATOM | 83 | CB | GLN | A | 153 | 76.561 | 85.778 | 71.760 | 1.00 | 39.17 | 6 |
| ATOM | 84 | CG | GLN | A | 153 | 75.949 | 84.483 | 72.295 | 1.00 | 44.50 | 6 |
| ATOM | 85 | CD | GLN | A | 153 | 74.608 | 84.148 | 71.656 | 1.00 | 48.57 | 6 |
| ATOM | 86 | OE1 | GLN | A | 153 | 73.986 | 83.131 | 71.981 | 1.00 | 49.71 | 8 |
| ATOM | 87 | NE2 | GLN | A | 153 | 74.157 | 85.002 | 70.742 | 1.00 | 49.54 | 7 |
| ATOM | 88 | C | GLN | A | 153 | 78.897 | 84.882 | 71.657 | 1.00 | 37.45 | 6 |
| ATOM | 89 | O | GLN | A | 153 | 79.309 | 83.987 | 72.399 | 1.00 | 35.62 | 8 |
| ATOM | 90 | N | LEU | A | 154 | 79.193 | 84.934 | 70.361 | 1.00 | 34.90 | 7 |
| ATOM | 91 | CA | LEU | A | 154 | 80.047 | 83.920 | 69.751 | 1.00 | 34.78 | 6 |
| ATOM | 92 | CB | LEU | A | 154 | 80.311 | 84.259 | 68.273 | 1.00 | 33.45 | 6 |
| ATOM | 93 | CG | LEU | A | 154 | 79.100 | 84.184 | 67.333 | 1.00 | 32.75 | 6 |
| ATOM | 94 | CD1 | LEU | A | 154 | 79.476 | 84.690 | 65.951 | 1.00 | 33.24 | 6 |
| ATOM | 95 | CD2 | LEU | A | 154 | 78.599 | 82.751 | 67.246 | 1.00 | 34.70 | 6 |
| ATOM | 96 | C | LEU | A | 154 | 81.364 | 83.853 | 70.532 | 1.00 | 32.82 | 6 |
| ATOM | 97 | O | LEU | A | 154 | 81.831 | 82.774 | 70.887 | 1.00 | 30.86 | 8 |
| ATOM | 98 | N | THR | A | 155 | 81.946 | 85.008 | 70.822 | 1.00 | 31.34 | 7 |
| ATOM | 99 | CA | THR | A | 155 | 83.200 | 85.051 | 71.573 | 1.00 | 31.85 | 6 |
| ATOM | 100 | CB | THR | A | 155 | 83.619 | 86.507 | 71.845 | 1.00 | 32.89 | 6 |
| ATOM | 101 | OG1 | THR | A | 155 | 83.931 | 87.137 | 70.598 | 1.00 | 32.79 | 8 |
| ATOM | 102 | CG2 | THR | A | 155 | 84.845 | 86.569 | 72.755 | 1.00 | 30.71 | 6 |
| ATOM | 103 | C | THR | A | 155 | 83.141 | 84.274 | 72.894 | 1.00 | 31.49 | 6 |
| ATOM | 104 | O | THR | A | 155 | 84.083 | 83.554 | 73.237 | 1.00 | 30.84 | 8 |
| ATOM | 105 | N | ALA | A | 156 | 82.037 | 84.397 | 73.625 | 1.00 | 29.88 | 7 |
| ATOM | 106 | CA | ALA | A | 156 | 81.901 | 83.696 | 74.900 | 1.00 | 30.23 | 6 |
| ATOM | 107 | CB | ALA | A | 156 | 80.731 | 84.274 | 75.700 | 1.00 | 31.61 | 6 |
| ATOM | 108 | C | ALA | A | 156 | 81.731 | 82.185 | 74.723 | 1.00 | 30.01 | 6 |
| ATOM | 109 | O | ALA | A | 156 | 82.153 | 81.401 | 75.578 | 1.00 | 30.85 | 8 |
| ATOM | 110 | N | LEU | A | 157 | 81.117 | 81.776 | 73.620 | 1.00 | 27.62 | 7 |
| ATOM | 111 | CA | LEU | A | 157 | 80.921 | 80.356 | 73.331 | 1.00 | 28.83 | 6 |
| ATOM | 112 | CB | LEU | A | 157 | 79.888 | 80.171 | 72.212 | 1.00 | 28.62 | 6 |
| ATOM | 113 | CG | LEU | A | 157 | 78.420 | 80.404 | 72.577 | 1.00 | 29.59 | 6 |
| ATOM | 114 | CD1 | LEU | A | 157 | 77.547 | 80.334 | 71.345 | 1.00 | 28.37 | 6 |
| ATOM | 115 | CD2 | LEU | A | 157 | 78.003 | 79.356 | 73.589 | 1.00 | 28.01 | 6 |
| ATOM | 116 | C | LEU | A | 157 | 82.238 | 79.718 | 72.895 | 1.00 | 29.34 | 6 |
| ATOM | 117 | O | LEU | A | 157 | 82.577 | 78.600 | 73.318 | 1.00 | 29.47 | 8 |
| ATOM | 118 | N | ILE | A | 158 | 82.962 | 80.444 | 72.045 | 1.00 | 28.70 | 7 |
| ATOM | 119 | CA | ILE | A | 158 | 84.241 | 80.014 | 71.493 | 1.00 | 28.13 | 6 |
| ATOM | 120 | CB | ILE | A | 158 | 84.735 | 81.004 | 70.410 | 1.00 | 31.27 | 6 |
| ATOM | 121 | CG2 | ILE | A | 158 | 86.089 | 80.550 | 69.859 | 1.00 | 29.81 | 6 |
| ATOM | 122 | CG1 | ILE | A | 158 | 83.706 | 81.127 | 69.269 | 1.00 | 29.92 | 6 |
| ATOM | 123 | CD1 | ILE | A | 158 | 83.645 | 79.951 | 68.361 | 1.00 | 36.11 | 6 |
| ATOM | 124 | C | ILE | A | 158 | 85.304 | 79.944 | 72.587 | 1.00 | 29.28 | 6 |
| ATOM | 125 | O | ILE | A | 158 | 86.031 | 78.957 | 72.703 | 1.00 | 26.55 | 8 |
| ATOM | 126 | N | GLY | A | 159 | 85.385 | 80.997 | 73.392 | 1.00 | 28.68 | 7 |
| ATOM | 127 | CA | GLY | A | 159 | 86.379 | 81.043 | 74.446 | 1.00 | 31.13 | 6 |
| ATOM | 128 | C | GLY | A | 159 | 87.588 | 81.769 | 73.898 | 1.00 | 31.36 | 6 |
| ATOM | 129 | O | GLY | A | 159 | 88.649 | 81.793 | 74.511 | 1.00 | 33.39 | 8 |
| ATOM | 130 | N | TYR | A | 160 | 87.415 | 82.358 | 72.720 | 1.00 | 31.49 | 7 |
| ATOM | 131 | CA | TYR | A | 160 | 88.476 | 83.102 | 72.052 | 1.00 | 31.09 | 6 |
| ATOM | 132 | CB | TYR | A | 160 | 89.356 | 82.146 | 71.232 | 1.00 | 30.13 | 6 |
| ATOM | 133 | CG | TYR | A | 160 | 90.676 | 82.752 | 70.792 | 1.00 | 30.00 | 6 |
| ATOM | 134 | CD1 | TYR | A | 160 | 91.763 | 82.821 | 71.673 | 1.00 | 31.76 | 6 |
| ATOM | 135 | CE1 | TYR | A | 160 | 92.967 | 83.423 | 71.293 | 1.00 | 29.41 | 6 |
| ATOM | 136 | CD2 | TYR | A | 160 | 90.823 | 83.299 | 69.520 | 1.00 | 29.39 | 6 |
| ATOM | 137 | CE2 | TYR | A | 160 | 92.015 | 83.904 | 69.128 | 1.00 | 31.70 | 6 |
| ATOM | 138 | CZ | TYR | A | 160 | 93.083 | 83.962 | 70.020 | 1.00 | 32.23 | 6 |
| ATOM | 139 | OH | TYR | A | 160 | 94.260 | 84.556 | 69.632 | 1.00 | 35.03 | 8 |
| ATOM | 140 | C | TYR | A | 160 | 87.821 | 84.120 | 71.117 | 1.00 | 31.93 | 6 |
| ATOM | 141 | O | TYR | A | 160 | 86.816 | 83.818 | 70.475 | 1.00 | 31.27 | 8 |
| ATOM | 142 | N | ASP | A | 161 | 88.383 | 85.321 | 71.033 | 1.00 | 33.22 | 7 |
| ATOM | 143 | CA | ASP | A | 161 | 87.815 | 86.341 | 70.153 | 1.00 | 36.53 | 6 |
| ATOM | 144 | CB | ASP | A | 161 | 87.940 | 87.731 | 70.784 | 1.00 | 36.69 | 6 |
| ATOM | 145 | CG | ASP | A | 161 | 87.509 | 88.843 | 69.838 | 1.00 | 39.02 | 6 |
| ATOM | 146 | OD1 | ASP | A | 161 | 86.627 | 88.612 | 68.981 | 1.00 | 34.49 | 8 |
| ATOM | 147 | OD2 | ASP | A | 161 | 88.049 | 89.961 | 69.963 | 1.00 | 43.82 | 8 |
| ATOM | 148 | C | ASP | A | 161 | 88.491 | 86.334 | 68.790 | 1.00 | 37.22 | 6 |
| ATOM | 149 | O | ASP | A | 161 | 89.610 | 86.830 | 68.640 | 1.00 | 37.03 | 8 |
| ATOM | 150 | N | VAL | A | 162 | 87.797 | 85.791 | 67.794 | 1.00 | 38.04 | 7 |
| ATOM | 151 | CA | VAL | A | 162 | 88.351 | 85.702 | 66.453 | 1.00 | 40.68 | 6 |
| ATOM | 152 | CB | VAL | A | 162 | 87.527 | 84.743 | 65.563 | 1.00 | 40.26 | 6 |
| ATOM | 153 | CG1 | VAL | A | 162 | 87.529 | 83.345 | 66.178 | 1.00 | 37.44 | 6 |
| ATOM | 154 | CG2 | VAL | A | 162 | 86.111 | 85.261 | 65.393 | 1.00 | 37.74 | 6 |
| ATOM | 155 | C | VAL | A | 162 | 88.505 | 87.038 | 65.737 | 1.00 | 43.93 | 6 |
| ATOM | 156 | O | VAL | A | 162 | 89.039 | 87.085 | 64.631 | 1.00 | 45.93 | 8 |
| ATOM | 157 | N | THR | A | 163 | 88.035 | 88.121 | 66.348 | 1.00 | 46.97 | 7 |
| ATOM | 158 | CA | THR | A | 163 | 88.196 | 89.435 | 65.728 | 1.00 | 49.88 | 6 |
| ATOM | 159 | CB | THR | A | 163 | 87.009 | 90.393 | 66.034 | 1.00 | 48.12 | 6 |
| ATOM | 160 | OG1 | THR | A | 163 | 87.036 | 90.782 | 67.412 | 1.00 | 47.75 | 8 |
| ATOM | 161 | CG2 | THR | A | 163 | 85.687 | 89.715 | 65.730 | 1.00 | 48.24 | 6 |
| ATOM | 162 | C | THR | A | 163 | 89.489 | 90.027 | 66.290 | 1.00 | 52.52 | 6 |
| ATOM | 163 | O | THR | A | 163 | 89.908 | 91.118 | 65.913 | 1.00 | 53.81 | 8 |
| ATOM | 164 | N | ASP | A | 164 | 90.116 | 89.281 | 67.194 | 1.00 | 55.39 | 7 |
| ATOM | 165 | CA | ASP | A | 164 | 91.369 | 89.700 | 67.813 | 1.00 | 57.64 | 6 |
| ATOM | 166 | CB | ASP | A | 164 | 91.578 | 88.950 | 69.132 | 1.00 | 60.50 | 6 |
| ATOM | 167 | CG | ASP | A | 164 | 92.927 | 89.244 | 69.769 | 1.00 | 62.49 | 6 |
| ATOM | 168 | OD1 | ASP | A | 164 | 93.114 | 90.369 | 70.283 | 1.00 | 63.75 | 8 |
| ATOM | 169 | OD2 | ASP | A | 164 | 93.801 | 88.349 | 69.750 | 1.00 | 63.20 | 8 |
| ATOM | 170 | C | ASP | A | 164 | 92.536 | 89.406 | 66.872 | 1.00 | 57.90 | 6 |
| ATOM | 171 | O | ASP | A | 164 | 92.810 | 88.247 | 66.557 | 1.00 | 58.81 | 8 |
| ATOM | 172 | N | VAL | A | 165 | 93.224 | 90.451 | 66.425 | 1.00 | 57.24 | 7 |
| ATOM | 173 | CA | VAL | A | 165 | 94.359 | 90.281 | 65.525 | 1.00 | 55.84 | 6 |
| ATOM | 174 | CB | VAL | A | 165 | 94.210 | 91.154 | 64.259 | 1.00 | 56.35 | 6 |
| ATOM | 175 | CG1 | VAL | A | 165 | 95.185 | 90.690 | 63.193 | 1.00 | 57.60 | 6 |
| ATOM | 176 | CG2 | VAL | A | 165 | 92.783 | 91.078 | 63.739 | 1.00 | 57.20 | 6 |
| ATOM | 177 | C | VAL | A | 165 | 95.624 | 90.683 | 66.263 | 1.00 | 54.43 | 6 |
| ATOM | 178 | O | VAL | A | 165 | 96.450 | 91.435 | 65.752 | 1.00 | 55.37 | 8 |
| ATOM | 179 | N | SER | A | 166 | 95.767 | 90.153 | 67.471 | 1.00 | 53.72 | 7 |
| ATOM | 180 | CA | SER | A | 166 | 96.904 | 90.447 | 68.336 | 1.00 | 52.28 | 6 |
| ATOM | 181 | CB | SER | A | 166 | 96.477 | 90.245 | 69.795 | 1.00 | 52.70 | 6 |
| ATOM | 182 | OG | SER | A | 166 | 97.472 | 90.694 | 70.694 | 1.00 | 56.25 | 8 |
| ATOM | 183 | C | SER | A | 166 | 98.179 | 89.633 | 68.057 | 1.00 | 50.66 | 6 |
| ATOM | 184 | O | SER | A | 166 | 99.297 | 90.128 | 68.245 | 1.00 | 51.68 | 8 |
| ATOM | 185 | N | ASN | A | 167 | 98.008 | 88.395 | 67.607 | 1.00 | 46.77 | 7 |
| ATOM | 186 | CA | ASN | A | 167 | 99.131 | 87.500 | 67.337 | 1.00 | 41.94 | 6 |
| ATOM | 187 | CB | ASN | A | 167 | 99.069 | 86.325 | 68.325 | 1.00 | 38.15 | 6 |
| ATOM | 188 | CG | ASN | A | 167 | 100.193 | 85.325 | 68.135 | 1.00 | 37.67 | 6 |
| ATOM | 189 | OD1 | ASN | A | 167 | 101.357 | 85.696 | 67.994 | 1.00 | 33.19 | 8 |
| ATOM | 190 | ND2 | ASN | A | 167 | 99.847 | 84.040 | 68.154 | 1.00 | 33.62 | 7 |
| ATOM | 191 | C | ASN | A | 167 | 99.077 | 87.000 | 65.893 | 1.00 | 40.79 | 6 |
| ATOM | 192 | O | ASN | A | 167 | 98.958 | 85.800 | 65.641 | 1.00 | 35.54 | 8 |
| ATOM | 193 | N | VAL | A | 168 | 99.164 | 87.932 | 64.946 | 1.00 | 40.77 | 7 |
| ATOM | 194 | CA | VAL | A | 168 | 99.113 | 87.590 | 63.531 | 1.00 | 41.64 | 6 |
| ATOM | 195 | CB | VAL | A | 168 | 97.714 | 87.865 | 62.954 | 1.00 | 41.95 | 6 |
| ATOM | 196 | CG1 | VAL | A | 168 | 97.589 | 87.243 | 61.595 | 1.00 | 44.58 | 6 |
| ATOM | 197 | CG2 | VAL | A | 168 | 96.650 | 87.315 | 63.873 | 1.00 | 43.63 | 6 |
| ATOM | 198 | C | VAL | A | 168 | 100.129 | 88.386 | 62.717 | 1.00 | 42.25 | 6 |
| ATOM | 199 | O | VAL | A | 168 | 100.522 | 89.488 | 63.103 | 1.00 | 43.63 | 8 |
| ATOM | 200 | N | HIS | A | 169 | 100.560 | 87.822 | 61.592 | 1.00 | 40.30 | 7 |
| ATOM | 201 | CA | HIS | A | 169 | 101.508 | 88.499 | 60.721 | 1.00 | 38.84 | 6 |
| ATOM | 202 | CB | HIS | A | 169 | 102.896 | 87.844 | 60.832 | 1.00 | 41.33 | 6 |
| ATOM | 203 | CG | HIS | A | 169 | 102.958 | 86.435 | 60.324 | 1.00 | 43.64 | 6 |
| ATOM | 204 | CD2 | HIS | A | 169 | 102.340 | 85.831 | 59.280 | 1.00 | 42.44 | 6 |
| ATOM | 205 | ND1 | HIS | A | 169 | 103.770 | 85.477 | 60.895 | 1.00 | 43.55 | 7 |
| ATOM | 206 | CE1 | HIS | A | 169 | 103.649 | 84.344 | 60.225 | 1.00 | 44.37 | 6 |
| ATOM | 207 | NE2 | HIS | A | 169 | 102.788 | 84.532 | 59.241 | 1.00 | 43.31 | 7 |
| ATOM | 208 | C | HIS | A | 169 | 101.015 | 88.514 | 59.265 | 1.00 | 36.27 | 6 |
| ATOM | 209 | O | HIS | A | 169 | 101.766 | 88.840 | 58.345 | 1.00 | 36.21 | 8 |
| ATOM | 210 | N | ASP | A | 170 | 99.748 | 88.154 | 59.068 | 1.00 | 32.58 | 7 |
| ATOM | 211 | CA | ASP | A | 170 | 99.137 | 88.144 | 57.742 | 1.00 | 31.52 | 6 |
| ATOM | 212 | CB | ASP | A | 170 | 99.689 | 86.984 | 56.890 | 1.00 | 33.07 | 6 |
| ATOM | 213 | CG | ASP | A | 170 | 99.198 | 85.612 | 57.342 | 1.00 | 35.23 | 6 |
| ATOM | 214 | OD1 | ASP | A | 170 | 98.628 | 85.483 | 58.449 | 1.00 | 35.14 | 8 |
| ATOM | 215 | OD2 | ASP | A | 170 | 99.398 | 84.645 | 56.577 | 1.00 | 39.13 | 8 |
| ATOM | 216 | C | ASP | A | 170 | 97.632 | 88.033 | 57.928 | 1.00 | 29.97 | 6 |
| ATOM | 217 | O | ASP | A | 170 | 97.155 | 88.027 | 59.056 | 1.00 | 31.02 | 8 |
| ATOM | 218 | N | ASP | A | 171 | 96.870 | 87.954 | 56.847 | 1.00 | 30.31 | 7 |
| ATOM | 219 | CA | ASP | A | 171 | 95.420 | 87.864 | 56.999 | 1.00 | 31.11 | 6 |
| ATOM | 220 | CB | ASP | A | 171 | 94.725 | 88.884 | 56.090 | 1.00 | 34.54 | 6 |
| ATOM | 221 | CG | ASP | A | 171 | 94.836 | 88.523 | 54.622 | 1.00 | 36.88 | 6 |
| ATOM | 222 | OD1 | ASP | A | 171 | 94.145 | 89.162 | 53.796 | 1.00 | 38.32 | 8 |
| ATOM | 223 | OD2 | ASP | A | 171 | 95.620 | 87.602 | 54.297 | 1.00 | 36.73 | 8 |
| ATOM | 224 | C | ASP | A | 171 | 94.854 | 86.474 | 56.704 | 1.00 | 29.95 | 6 |
| ATOM | 225 | O | ASP | A | 171 | 93.721 | 86.351 | 56.232 | 1.00 | 30.44 | 8 |
| ATOM | 226 | N | GLU | A | 172 | 95.604 | 85.416 | 56.983 | 1.00 | 28.56 | 7 |
| ATOM | 227 | CA | GLU | A | 172 | 95.044 | 84.104 | 56.670 | 1.00 | 26.65 | 6 |
| ATOM | 228 | CB | GLU | A | 172 | 96.064 | 82.982 | 56.871 | 1.00 | 26.23 | 6 |
| ATOM | 229 | CG | GLU | A | 172 | 95.606 | 81.689 | 56.190 | 1.00 | 25.27 | 6 |
| ATOM | 230 | CD | GLU | A | 172 | 96.448 | 80.479 | 56.538 | 1.00 | 26.71 | 6 |
| ATOM | 231 | OE1 | GLU | A | 172 | 97.515 | 80.637 | 57.162 | 1.00 | 25.24 | 8 |
| ATOM | 232 | OE2 | GLU | A | 172 | 96.034 | 79.360 | 56.174 | 1.00 | 24.99 | 8 |
| ATOM | 233 | C | GLU | A | 172 | 93.773 | 83.794 | 57.460 | 1.00 | 25.87 | 6 |
| ATOM | 234 | O | GLU | A | 172 | 92.801 | 83.279 | 56.897 | 1.00 | 24.66 | 8 |
| ATOM | 235 | N | LEU | A | 173 | 93.769 | 84.110 | 58.756 | 1.00 | 26.09 | 7 |
| ATOM | 236 | CA | LEU | A | 173 | 92.601 | 83.846 | 59.601 | 1.00 | 28.17 | 6 |
| ATOM | 237 | CB | LEU | A | 173 | 92.901 | 84.213 | 61.068 | 1.00 | 29.49 | 6 |
| ATOM | 238 | CG | LEU | A | 173 | 93.949 | 83.351 | 61.785 | 1.00 | 30.42 | 6 |
| ATOM | 239 | CD1 | LEU | A | 173 | 94.470 | 84.071 | 63.025 | 1.00 | 31.22 | 6 |
| ATOM | 240 | CD2 | LEU | A | 173 | 93.333 | 81.999 | 62.166 | 1.00 | 30.93 | 6 |
| ATOM | 241 | C | LEU | A | 173 | 91.358 | 84.588 | 59.106 | 1.00 | 29.38 | 6 |
| ATOM | 242 | O | LEU | A | 173 | 90.288 | 83.996 | 59.012 | 1.00 | 28.61 | 8 |
| ATOM | 243 | N | GLU | A | 174 | 91.486 | 85.876 | 58.785 | 1.00 | 30.24 | 7 |
| ATOM | 244 | CA | GLU | A | 174 | 90.329 | 86.625 | 58.277 | 1.00 | 32.47 | 6 |
| ATOM | 245 | CB | GLU | A | 174 | 90.679 | 88.099 | 58.050 | 1.00 | 35.00 | 6 |
| ATOM | 246 | CG | GLU | A | 174 | 89.984 | 89.076 | 58.994 | 1.00 | 40.36 | 6 |
| ATOM | 247 | CD | GLU | A | 174 | 88.492 | 88.813 | 59.146 | 1.00 | 40.72 | 6 |
| ATOM | 248 | OE1 | GLU | A | 174 | 87.765 | 88.774 | 58.124 | 1.00 | 41.42 | 8 |
| ATOM | 249 | OE2 | GLU | A | 174 | 88.043 | 88.656 | 60.299 | 1.00 | 40.30 | 8 |
| ATOM | 250 | C | GLU | A | 174 | 89.908 | 86.015 | 56.938 | 1.00 | 30.38 | 6 |
| ATOM | 251 | O | GLU | A | 174 | 88.728 | 85.878 | 56.639 | 1.00 | 31.05 | 8 |
| ATOM | 252 | N | PHE | A | 175 | 90.903 | 85.659 | 56.140 | 1.00 | 30.71 | 7 |
| ATOM | 253 | CA | PHE | A | 175 | 90.683 | 85.049 | 54.838 | 1.00 | 32.03 | 6 |
| ATOM | 254 | CB | PHE | A | 175 | 92.043 | 84.752 | 54.196 | 1.00 | 35.09 | 6 |
| ATOM | 255 | CG | PHE | A | 175 | 91.961 | 84.202 | 52.795 | 1.00 | 38.72 | 6 |
| ATOM | 256 | CD1 | PHE | A | 175 | 91.707 | 85.043 | 51.713 | 1.00 | 39.86 | 6 |
| ATOM | 257 | CD2 | PHE | A | 175 | 92.158 | 82.844 | 52.557 | 1.00 | 39.25 | 6 |
| ATOM | 258 | CE1 | PHE | A | 175 | 91.657 | 84.538 | 50.416 | 1.00 | 41.39 | 6 |
| ATOM | 259 | CE2 | PHE | A | 175 | 92.109 | 82.328 | 51.263 | 1.00 | 41.71 | 6 |
| ATOM | 260 | CZ | PHE | A | 175 | 91.858 | 83.176 | 50.191 | 1.00 | 41.41 | 6 |
| ATOM | 261 | C | PHE | A | 175 | 89.892 | 83.748 | 55.027 | 1.00 | 31.25 | 6 |
| ATOM | 262 | O | PHE | A | 175 | 88.981 | 83.432 | 54.263 | 1.00 | 30.51 | 8 |
| ATOM | 263 | N | THR | A | 176 | 90.248 | 82.992 | 56.055 | 1.00 | 29.53 | 7 |
| ATOM | 264 | CA | THR | A | 176 | 89.568 | 81.738 | 56.318 | 1.00 | 27.79 | 6 |
| ATOM | 265 | CB | THR | A | 176 | 90.375 | 80.904 | 57.319 | 1.00 | 27.60 | 6 |
| ATOM | 266 | OG1 | THR | A | 176 | 91.638 | 80.579 | 56.721 | 1.00 | 23.25 | 8 |
| ATOM | 267 | CG2 | THR | A | 176 | 89.628 | 79.619 | 57.685 | 1.00 | 25.40 | 6 |
| ATOM | 268 | C | THR | A | 176 | 88.127 | 81.953 | 56.785 | 1.00 | 27.11 | 6 |
| ATOM | 269 | O | THR | A | 176 | 87.219 | 81.252 | 56.337 | 1.00 | 27.55 | 8 |
| ATOM | 270 | N | ARG | A | 177 | 87.901 | 82.935 | 57.653 | 1.00 | 26.93 | 7 |
| ATOM | 271 | CA | ARG | A | 177 | 86.534 | 83.214 | 58.109 | 1.00 | 27.84 | 6 |
| ATOM | 272 | CB | ARG | A | 177 | 86.498 | 84.436 | 59.035 | 1.00 | 25.62 | 6 |
| ATOM | 273 | CG | ARG | A | 177 | 87.341 | 84.335 | 60.298 | 1.00 | 29.23 | 6 |
| ATOM | 274 | CD | ARG | A | 177 | 87.188 | 85.612 | 61.127 | 1.00 | 30.71 | 6 |
| ATOM | 275 | NE | ARG | A | 177 | 85.822 | 85.741 | 61.634 | 1.00 | 33.35 | 7 |
| ATOM | 276 | CZ | ARG | A | 177 | 85.200 | 86.896 | 61.860 | 1.00 | 36.50 | 6 |
| ATOM | 277 | NH1 | ARG | A | 177 | 85.812 | 88.052 | 61.627 | 1.00 | 36.25 | 7 |
| ATOM | 278 | NH2 | ARG | A | 177 | 83.956 | 86.895 | 62.320 | 1.00 | 36.96 | 7 |
| ATOM | 279 | C | ARG | A | 177 | 85.651 | 83.490 | 56.883 | 1.00 | 29.24 | 6 |
| ATOM | 280 | O | ARG | A | 177 | 84.533 | 82.976 | 56.778 | 1.00 | 29.65 | 8 |
| ATOM | 281 | N | ARG | A | 178 | 86.150 | 84.303 | 55.955 | 1.00 | 29.32 | 7 |
| ATOM | 282 | CA | ARG | A | 178 | 85.376 | 84.612 | 54.750 | 1.00 | 33.07 | 6 |
| ATOM | 283 | CB | ARG | A | 178 | 86.090 | 85.667 | 53.902 | 1.00 | 34.30 | 6 |
| ATOM | 284 | CG | ARG | A | 178 | 86.228 | 87.031 | 54.554 | 1.00 | 39.30 | 6 |
| ATOM | 285 | CD | ARG | A | 178 | 86.838 | 88.015 | 53.571 | 1.00 | 44.34 | 6 |
| ATOM | 286 | NE | ARG | A | 178 | 86.969 | 89.359 | 54.126 | 1.00 | 49.81 | 7 |
| ATOM | 287 | CZ | ARG | A | 178 | 87.777 | 89.675 | 55.133 | 1.00 | 52.73 | 6 |
| ATOM | 288 | NH1 | ARG | A | 178 | 88.527 | 88.737 | 55.699 | 1.00 | 54.48 | 7 |
| ATOM | 289 | NH2 | ARG | A | 178 | 87.848 | 90.929 | 55.566 | 1.00 | 52.35 | 7 |
| ATOM | 290 | C | ARG | A | 178 | 85.160 | 83.353 | 53.907 | 1.00 | 32.02 | 6 |
| ATOM | 291 | O | ARG | A | 178 | 84.057 | 83.083 | 53.431 | 1.00 | 31.99 | 8 |
| ATOM | 292 | N | GLY | A | 179 | 86.223 | 82.576 | 53.732 | 1.00 | 32.13 | 7 |
| ATOM | 293 | CA | GLY | A | 179 | 86.123 | 81.365 | 52.935 | 1.00 | 31.44 | 6 |
| ATOM | 294 | C | GLY | A | 179 | 85.145 | 80.311 | 53.424 | 1.00 | 30.96 | 6 |
| ATOM | 295 | O | GLY | A | 179 | 84.570 | 79.584 | 52.616 | 1.00 | 30.97 | 8 |
| ATOM | 296 | N | LEU | A | 180 | 84.947 | 80.211 | 54.737 | 1.00 | 29.65 | 7 |
| ATOM | 297 | CA | LEU | A | 180 | 84.042 | 79.192 | 55.264 | 1.00 | 30.64 | 6 |
| ATOM | 298 | CB | LEU | A | 180 | 84.433 | 78.842 | 56.707 | 1.00 | 28.91 | 6 |
| ATOM | 299 | CG | LEU | A | 180 | 85.874 | 78.336 | 56.827 | 1.00 | 27.68 | 6 |
| ATOM | 300 | CD1 | LEU | A | 180 | 86.207 | 78.064 | 58.290 | 1.00 | 22.85 | 6 |
| ATOM | 301 | CD2 | LEU | A | 180 | 86.052 | 77.064 | 55.992 | 1.00 | 24.81 | 6 |
| ATOM | 302 | C | LEU | A | 180 | 82.557 | 79.543 | 55.178 | 1.00 | 29.37 | 6 |
| ATOM | 303 | O | LEU | A | 180 | 81.698 | 78.706 | 55.461 | 1.00 | 29.31 | 8 |
| ATOM | 304 | N | VAL | A | 181 | 82.254 | 80.773 | 54.771 | 1.00 | 32.29 | 7 |
| ATOM | 305 | CA | VAL | A | 181 | 80.861 | 81.198 | 54.624 | 1.00 | 33.26 | 6 |
| ATOM | 306 | CB | VAL | A | 181 | 80.763 | 82.666 | 54.141 | 1.00 | 34.48 | 6 |
| ATOM | 307 | CG1 | VAL | A | 181 | 79.358 | 82.956 | 53.626 | 1.00 | 35.84 | 6 |
| ATOM | 308 | CG2 | VAL | A | 181 | 81.093 | 83.612 | 55.288 | 1.00 | 35.87 | 6 |
| ATOM | 309 | C | VAL | A | 181 | 80.118 | 80.301 | 53.633 | 1.00 | 32.12 | 6 |
| ATOM | 310 | O | VAL | A | 181 | 79.062 | 79.758 | 53.944 | 1.00 | 30.27 | 8 |
| ATOM | 311 | N | THR | A | 182 | 80.674 | 80.140 | 52.440 | 1.00 | 33.84 | 7 |
| ATOM | 312 | CA | THR | A | 182 | 80.047 | 79.304 | 51.418 | 1.00 | 34.31 | 6 |
| ATOM | 313 | CB | THR | A | 182 | 80.925 | 79.251 | 50.136 | 1.00 | 35.76 | 6 |
| ATOM | 314 | OG1 | THR | A | 182 | 80.967 | 80.551 | 49.534 | 1.00 | 34.92 | 8 |
| ATOM | 315 | CG2 | THR | A | 182 | 80.365 | 78.242 | 49.132 | 1.00 | 35.91 | 6 |
| ATOM | 316 | C | THR | A | 182 | 79.766 | 77.871 | 51.894 | 1.00 | 34.51 | 6 |
| ATOM | 317 | O | THR | A | 182 | 78.632 | 77.392 | 51.811 | 1.00 | 36.33 | 8 |
| ATOM | 318 | N | PRO | A | 183 | 80.794 | 77.165 | 52.401 | 1.00 | 33.19 | 7 |
| ATOM | 319 | CD | PRO | A | 183 | 82.198 | 77.585 | 52.542 | 1.00 | 33.25 | 6 |
| ATOM | 320 | CA | PRO | A | 183 | 80.616 | 75.787 | 52.878 | 1.00 | 30.93 | 6 |
| ATOM | 321 | CB | PRO | A | 183 | 81.976 | 75.458 | 53.488 | 1.00 | 33.18 | 6 |
| ATOM | 322 | CG | PRO | A | 183 | 82.912 | 76.261 | 52.661 | 1.00 | 34.43 | 6 |
| ATOM | 323 | C | PRO | A | 183 | 79.488 | 75.672 | 53.896 | 1.00 | 28.42 | 6 |
| ATOM | 324 | O | PRO | A | 183 | 78.671 | 74.746 | 53.846 | 1.00 | 27.52 | 8 |
| ATOM | 325 | N | ARG | A | 184 | 79.451 | 76.618 | 54.824 | 1.00 | 27.37 | 7 |
| ATOM | 326 | CA | ARG | A | 184 | 78.422 | 76.633 | 55.859 | 1.00 | 29.33 | 6 |
| ATOM | 327 | CB | ARG | A | 184 | 78.728 | 77.710 | 56.905 | 1.00 | 27.77 | 6 |
| ATOM | 328 | CG | ARG | A | 184 | 77.528 | 78.084 | 57.765 | 1.00 | 30.74 | 6 |
| ATOM | 329 | CD | ARG | A | 184 | 77.884 | 79.150 | 58.781 | 1.00 | 29.78 | 6 |
| ATOM | 330 | NE | ARG | A | 184 | 78.764 | 80.172 | 58.233 | 1.00 | 37.34 | 7 |
| ATOM | 331 | CZ | ARG | A | 184 | 78.375 | 81.414 | 57.960 | 1.00 | 38.97 | 6 |
| ATOM | 332 | NH1 | ARG | A | 184 | 77.122 | 81.777 | 58.196 | 1.00 | 46.41 | 7 |
| ATOM | 333 | NH2 | ARG | A | 184 | 79.222 | 82.290 | 57.442 | 1.00 | 35.65 | 7 |
| ATOM | 334 | C | ARG | A | 184 | 77.027 | 76.882 | 55.289 | 1.00 | 30.29 | 6 |
| ATOM | 335 | O | ARG | A | 184 | 76.105 | 76.101 | 55.526 | 1.00 | 30.35 | 8 |
| ATOM | 336 | N | MET | A | 185 | 76.868 | 77.976 | 54.549 | 1.00 | 32.41 | 7 |
| ATOM | 337 | CA | MET | A | 185 | 75.555 | 78.296 | 53.985 | 1.00 | 34.11 | 6 |
| ATOM | 338 | CB | MET | A | 185 | 75.618 | 79.595 | 53.175 | 1.00 | 35.65 | 6 |
| ATOM | 339 | CG | MET | A | 185 | 76.042 | 80.805 | 53.994 | 1.00 | 41.17 | 6 |
| ATOM | 340 | SD | MET | A | 185 | 75.773 | 82.377 | 53.135 | 1.00 | 49.08 | 16 |
| ATOM | 341 | CE | MET | A | 185 | 76.863 | 82.178 | 51.722 | 1.00 | 47.55 | 6 |
| ATOM | 342 | C | MET | A | 185 | 75.018 | 77.164 | 53.121 | 1.00 | 32.33 | 6 |
| ATOM | 343 | O | MET | A | 185 | 73.834 | 76.849 | 53.178 | 1.00 | 32.67 | 8 |
| ATOM | 344 | N | ALA | A | 186 | 75.894 | 76.541 | 52.337 | 1.00 | 31.91 | 7 |
| ATOM | 345 | CA | ALA | A | 186 | 75.488 | 75.444 | 51.465 | 1.00 | 31.35 | 6 |
| ATOM | 346 | CB | ALA | A | 186 | 76.654 | 75.007 | 50.600 | 1.00 | 31.31 | 6 |
| ATOM | 347 | C | ALA | A | 186 | 74.947 | 74.257 | 52.254 | 1.00 | 31.91 | 6 |
| ATOM | 348 | O | ALA | A | 186 | 73.943 | 73.655 | 51.866 | 1.00 | 32.67 | 8 |
| ATOM | 349 | N | GLU | A | 187 | 75.607 | 73.906 | 53.358 | 1.00 | 31.90 | 7 |
| ATOM | 350 | CA | GLU | A | 187 | 75.133 | 72.787 | 54.165 | 1.00 | 31.05 | 6 |
| ATOM | 351 | CB | GLU | A | 187 | 76.191 | 72.374 | 55.200 | 1.00 | 31.16 | 6 |
| ATOM | 352 | CG | GLU | A | 187 | 75.843 | 71.096 | 55.945 | 1.00 | 31.96 | 6 |
| ATOM | 353 | CD | GLU | A | 187 | 75.594 | 69.917 | 55.010 | 1.00 | 33.62 | 6 |
| ATOM | 354 | OE1 | GLU | A | 187 | 76.476 | 69.612 | 54.175 | 1.00 | 32.31 | 8 |
| ATOM | 355 | OE2 | GLU | A | 187 | 74.518 | 69.295 | 55.122 | 1.00 | 32.68 | 8 |
| ATOM | 356 | C | GLU | A | 187 | 73.825 | 73.162 | 54.872 | 1.00 | 31.33 | 6 |
| ATOM | 357 | O | GLU | A | 187 | 72.914 | 72.346 | 54.980 | 1.00 | 31.19 | 8 |
| ATOM | 358 | N | VAL | A | 188 | 73.731 | 74.402 | 55.343 | 1.00 | 31.77 | 7 |
| ATOM | 359 | CA | VAL | A | 188 | 72.517 | 74.865 | 56.020 | 1.00 | 31.65 | 6 |
| ATOM | 360 | CB | VAL | A | 188 | 72.678 | 76.322 | 56.520 | 1.00 | 32.04 | 6 |
| ATOM | 361 | CG1 | VAL | A | 188 | 71.320 | 76.886 | 56.961 | 1.00 | 32.24 | 6 |
| ATOM | 362 | CG2 | VAL | A | 188 | 73.669 | 76.366 | 57.671 | 1.00 | 30.03 | 6 |
| ATOM | 363 | C | VAL | A | 188 | 71.291 | 74.798 | 55.103 | 1.00 | 33.16 | 6 |
| ATOM | 364 | O | VAL | A | 188 | 70.210 | 74.406 | 55.530 | 1.00 | 34.73 | 8 |
| ATOM | 365 | N | ALA | A | 189 | 71.457 | 75.184 | 53.844 | 1.00 | 34.76 | 7 |
| ATOM | 366 | CA | ALA | A | 189 | 70.344 | 75.161 | 52.895 | 1.00 | 36.39 | 6 |
| ATOM | 367 | CB | ALA | A | 189 | 70.672 | 76.029 | 51.686 | 1.00 | 33.11 | 6 |
| ATOM | 368 | C | ALA | A | 189 | 70.041 | 73.745 | 52.438 | 1.00 | 37.88 | 6 |
| ATOM | 369 | O | ALA | A | 189 | 68.949 | 73.459 | 51.941 | 1.00 | 39.60 | 8 |
| ATOM | 370 | N | SER | A | 190 | 71.005 | 72.853 | 52.618 | 1.00 | 38.95 | 7 |
| ATOM | 371 | CA | SER | A | 190 | 70.847 | 71.474 | 52.177 | 1.00 | 39.35 | 6 |
| ATOM | 372 | CB | SER | A | 190 | 72.218 | 70.915 | 51.788 | 1.00 | 39.59 | 6 |
| ATOM | 373 | OG | SER | A | 190 | 72.109 | 69.590 | 51.311 | 1.00 | 40.87 | 8 |
| ATOM | 374 | C | SER | A | 190 | 70.174 | 70.525 | 53.165 | 1.00 | 39.40 | 6 |
| ATOM | 375 | O | SER | A | 190 | 69.494 | 69.580 | 52.761 | 1.00 | 39.50 | 8 |
| ATOM | 376 | N | ARG | A | 191 | 70.344 | 70.777 | 54.454 | 1.00 | 39.21 | 7 |
| ATOM | 377 | CA | ARG | A | 191 | 69.776 | 69.888 | 55.458 | 1.00 | 38.96 | 6 |
| ATOM | 378 | CB | ARG | A | 191 | 70.443 | 70.138 | 56.814 | 1.00 | 38.48 | 6 |
| ATOM | 379 | CG | ARG | A | 191 | 70.107 | 71.477 | 57.440 | 1.00 | 38.08 | 6 |
| ATOM | 380 | CD | ARG | A | 191 | 71.013 | 71.773 | 58.633 | 1.00 | 35.79 | 6 |
| ATOM | 381 | NE | ARG | A | 191 | 70.569 | 72.963 | 59.343 | 1.00 | 33.38 | 7 |
| ATOM | 382 | CZ | ARG | A | 191 | 71.342 | 73.711 | 60.120 | 1.00 | 33.32 | 6 |
| ATOM | 383 | NH1 | ARG | A | 191 | 72.621 | 73.394 | 60.298 | 1.00 | 32.02 | 7 |
| ATOM | 384 | NH2 | ARG | A | 191 | 70.833 | 74.783 | 60.716 | 1.00 | 30.46 | 7 |
| ATOM | 385 | C | ARG | A | 191 | 68.258 | 69.947 | 55.608 | 1.00 | 39.45 | 6 |
| ATOM | 386 | O | ARG | A | 191 | 67.636 | 71.006 | 55.476 | 1.00 | 39.71 | 8 |
| ATOM | 387 | N | ASP | A | 192 | 67.679 | 68.783 | 55.878 | 1.00 | 38.00 | 7 |
| ATOM | 388 | CA | ASP | A | 192 | 66.245 | 68.629 | 56.075 | 1.00 | 39.84 | 6 |
| ATOM | 389 | CB | ASP | A | 192 | 65.870 | 67.148 | 55.963 | 1.00 | 40.03 | 6 |
| ATOM | 390 | CG | ASP | A | 192 | 64.409 | 66.889 | 56.248 | 1.00 | 43.15 | 6 |
| ATOM | 391 | OD1 | ASP | A | 192 | 63.555 | 67.343 | 55.457 | 1.00 | 45.87 | 8 |
| ATOM | 392 | OD2 | ASP | A | 192 | 64.113 | 66.232 | 57.266 | 1.00 | 45.16 | 8 |
| ATOM | 393 | C | ASP | A | 192 | 65.891 | 69.152 | 57.467 | 1.00 | 40.43 | 6 |
| ATOM | 394 | O | ASP | A | 192 | 66.370 | 68.631 | 58.474 | 1.00 | 38.03 | 8 |
| ATOM | 395 | N | PRO | A | 193 | 65.044 | 70.189 | 57.541 | 1.00 | 41.55 | 7 |
| ATOM | 396 | CD | PRO | A | 193 | 64.320 | 70.847 | 56.441 | 1.00 | 42.01 | 6 |
| ATOM | 397 | CA | PRO | A | 193 | 64.656 | 70.756 | 58.836 | 1.00 | 40.65 | 6 |
| ATOM | 398 | CB | PRO | A | 193 | 63.666 | 71.858 | 58.453 | 1.00 | 41.29 | 6 |
| ATOM | 399 | CG | PRO | A | 193 | 63.107 | 71.385 | 57.148 | 1.00 | 43.85 | 6 |
| ATOM | 400 | C | PRO | A | 193 | 64.079 | 69.755 | 59.835 | 1.00 | 40.66 | 6 |
| ATOM | 401 | O | PRO | A | 193 | 64.408 | 69.802 | 61.025 | 1.00 | 39.99 | 8 |
| ATOM | 402 | N | LYS | A | 194 | 63.233 | 68.847 | 59.362 | 1.00 | 39.17 | 7 |
| ATOM | 403 | CA | LYS | A | 194 | 62.640 | 67.859 | 60.248 | 1.00 | 39.32 | 6 |
| ATOM | 404 | CB | LYS | A | 194 | 61.497 | 67.123 | 59.539 | 1.00 | 42.18 | 6 |
| ATOM | 405 | CG | LYS | A | 194 | 60.188 | 67.917 | 59.542 | 1.00 | 46.80 | 6 |
| ATOM | 406 | CD | LYS | A | 194 | 59.103 | 67.305 | 58.655 | 1.00 | 48.93 | 6 |
| ATOM | 407 | CE | LYS | A | 194 | 59.305 | 67.673 | 57.185 | 1.00 | 50.15 | 6 |
| ATOM | 408 | NZ | LYS | A | 194 | 58.293 | 67.047 | 56.289 | 1.00 | 49.54 | 7 |
| ATOM | 409 | C | LYS | A | 194 | 63.663 | 66.864 | 60.788 | 1.00 | 38.24 | 6 |
| ATOM | 410 | O | LYS | A | 194 | 63.715 | 66.626 | 61.998 | 1.00 | 38.44 | 8 |
| ATOM | 411 | N | LEU | A | 195 | 64.480 | 66.289 | 59.911 | 1.00 | 34.90 | 7 |
| ATOM | 412 | CA | LEU | A | 195 | 65.481 | 65.324 | 60.354 | 1.00 | 33.46 | 6 |
| ATOM | 413 | CB | LEU | A | 195 | 66.031 | 64.531 | 59.171 | 1.00 | 34.18 | 6 |
| ATOM | 414 | CG | LEU | A | 195 | 65.118 | 63.410 | 58.671 | 1.00 | 37.54 | 6 |
| ATOM | 415 | CD1 | LEU | A | 195 | 65.680 | 62.817 | 57.387 | 1.00 | 38.35 | 6 |
| ATOM | 416 | CD2 | LEU | A | 195 | 64.984 | 62.341 | 59.749 | 1.00 | 36.51 | 6 |
| ATOM | 417 | C | LEU | A | 195 | 66.631 | 65.968 | 61.114 | 1.00 | 30.93 | 6 |
| ATOM | 418 | O | LEU | A | 195 | 67.303 | 65.308 | 61.891 | 1.00 | 31.92 | 8 |
| ATOM | 419 | N | TYR | A | 196 | 66.861 | 67.254 | 60.892 | 1.00 | 30.29 | 7 |
| ATOM | 420 | CA | TYR | A | 196 | 67.938 | 67.938 | 61.585 | 1.00 | 30.25 | 6 |
| ATOM | 421 | CB | TYR | A | 196 | 68.242 | 69.280 | 60.920 | 1.00 | 28.95 | 6 |
| ATOM | 422 | CG | TYR | A | 196 | 69.341 | 70.064 | 61.619 | 1.00 | 30.60 | 6 |
| ATOM | 423 | CD1 | TYR | A | 196 | 70.633 | 69.538 | 61.742 | 1.00 | 27.81 | 6 |
| ATOM | 424 | CE1 | TYR | A | 196 | 71.649 | 70.252 | 62.383 | 1.00 | 25.05 | 6 |
| ATOM | 425 | CD2 | TYR | A | 196 | 69.092 | 71.328 | 62.155 | 1.00 | 26.98 | 6 |
| ATOM | 426 | CE2 | TYR | A | 196 | 70.103 | 72.054 | 62.797 | 1.00 | 26.28 | 6 |
| ATOM | 427 | CZ | TYR | A | 196 | 71.380 | 71.510 | 62.908 | 1.00 | 26.79 | 6 |
| ATOM | 428 | OH | TYR | A | 196 | 72.386 | 72.219 | 63.542 | 1.00 | 26.23 | 8 |
| ATOM | 429 | C | TYR | A | 196 | 67.504 | 68.168 | 63.025 | 1.00 | 31.75 | 6 |
| ATOM | 430 | O | TYR | A | 196 | 68.302 | 68.067 | 63.962 | 1.00 | 29.91 | 8 |
| ATOM | 431 | N | ALA | A | 197 | 66.216 | 68.457 | 63.185 | 1.00 | 31.94 | 7 |
| ATOM | 432 | CA | ALA | A | 197 | 65.631 | 68.725 | 64.495 | 1.00 | 32.73 | 6 |
| ATOM | 433 | CB | ALA | A | 197 | 64.210 | 69.303 | 64.318 | 1.00 | 30.99 | 6 |
| ATOM | 434 | C | ALA | A | 197 | 65.590 | 67.510 | 65.415 | 1.00 | 31.74 | 6 |
| ATOM | 435 | O | ALA | A | 197 | 65.821 | 67.627 | 66.620 | 1.00 | 30.59 | 8 |
| ATOM | 436 | N | MET | A | 198 | 65.306 | 66.342 | 64.851 | 1.00 | 31.75 | 7 |
| ATOM | 437 | CA | MET | A | 198 | 65.204 | 65.134 | 65.656 | 1.00 | 30.86 | 6 |
| ATOM | 438 | CB | MET | A | 198 | 63.992 | 64.321 | 65.194 | 1.00 | 35.44 | 6 |
| ATOM | 439 | CG | MET | A | 198 | 62.678 | 65.078 | 65.343 | 1.00 | 38.40 | 6 |
| ATOM | 440 | SD | MET | A | 198 | 61.238 | 64.104 | 64.876 | 1.00 | 41.71 | 16 |
| ATOM | 441 | CE | MET | A | 198 | 61.265 | 64.311 | 63.089 | 1.00 | 42.99 | 6 |
| ATOM | 442 | C | MET | A | 198 | 66.441 | 64.247 | 65.698 | 1.00 | 30.86 | 6 |
| ATOM | 443 | O | MET | A | 198 | 66.467 | 63.267 | 66.435 | 1.00 | 28.18 | 8 |
| ATOM | 444 | N | HIS | A | 199 | 67.454 | 64.593 | 64.905 | 1.00 | 31.12 | 7 |
| ATOM | 445 | CA | HIS | A | 199 | 68.723 | 63.858 | 64.842 | 1.00 | 31.16 | 6 |
| ATOM | 446 | CB | HIS | A | 199 | 69.744 | 64.500 | 65.795 | 1.00 | 31.34 | 6 |
| ATOM | 447 | CG | HIS | A | 199 | 69.302 | 64.525 | 67.224 | 1.00 | 32.13 | 6 |
| ATOM | 448 | CD2 | HIS | A | 199 | 69.604 | 63.713 | 68.265 | 1.00 | 34.85 | 6 |
| ATOM | 449 | ND1 | HIS | A | 199 | 68.393 | 65.443 | 67.707 | 1.00 | 33.92 | 7 |
| ATOM | 450 | CE1 | HIS | A | 199 | 68.155 | 65.193 | 68.982 | 1.00 | 34.56 | 6 |
| ATOM | 451 | NE2 | HIS | A | 199 | 68.875 | 64.148 | 69.345 | 1.00 | 33.68 | 7 |
| ATOM | 452 | C | HIS | A | 199 | 68.681 | 62.354 | 65.126 | 1.00 | 31.00 | 6 |
| ATOM | 453 | O | HIS | A | 199 | 69.395 | 61.858 | 66.005 | 1.00 | 32.05 | 8 |
| ATOM | 454 | N | PRO | A | 200 | 67.527 | 62.048 | 64.298 | 1.00 | 29.80 | 7 |
| ATOM | 455 | CD | PRO | A | 200 | 66.759 | 62.440 | 63.104 | 1.00 | 28.73 | 6 |
| ATOM | 456 | CA | PRO | A | 200 | 67.429 | 60.608 | 64.547 | 1.00 | 29.05 | 6 |
| ATOM | 457 | CB | PRO | A | 200 | 66.495 | 60.122 | 63.433 | 1.00 | 30.89 | 6 |
| ATOM | 458 | CG | PRO | A | 200 | 66.679 | 61.138 | 62.345 | 1.00 | 30.13 | 6 |
| ATOM | 459 | C | PRO | A | 200 | 68.797 | 59.926 | 64.502 | 1.00 | 30.31 | 6 |
| ATOM | 460 | O | PRO | A | 200 | 69.615 | 60.220 | 63.633 | 1.00 | 30.28 | 8 |
| ATOM | 461 | N | TRP | A | 201 | 69.054 | 59.028 | 65.445 | 1.00 | 32.21 | 7 |
| ATOM | 462 | CA | TRP | A | 201 | 70.336 | 58.329 | 65.476 | 1.00 | 33.69 | 6 |
| ATOM | 463 | CB | TRP | A | 201 | 70.875 | 58.230 | 66.910 | 1.00 | 34.08 | 6 |
| ATOM | 464 | CG | TRP | A | 201 | 72.292 | 57.711 | 66.993 | 1.00 | 37.06 | 6 |
| ATOM | 465 | CD2 | TRP | A | 201 | 73.040 | 57.428 | 68.185 | 1.00 | 35.36 | 6 |
| ATOM | 466 | CE2 | TRP | A | 201 | 74.314 | 56.971 | 67.782 | 1.00 | 37.70 | 6 |
| ATOM | 467 | CE3 | TRP | A | 201 | 72.757 | 57.519 | 69.553 | 1.00 | 36.21 | 6 |
| ATOM | 468 | CD1 | TRP | A | 201 | 73.126 | 57.420 | 65.943 | 1.00 | 37.66 | 6 |
| ATOM | 469 | NE1 | TRP | A | 201 | 74.339 | 56.974 | 66.412 | 1.00 | 38.14 | 7 |
| ATOM | 470 | CZ2 | TRP | A | 201 | 75.307 | 56.602 | 68.702 | 1.00 | 36.84 | 6 |
| ATOM | 471 | CZ3 | TRP | A | 201 | 73.745 | 57.152 | 70.471 | 1.00 | 37.14 | 6 |
| ATOM | 472 | CH2 | TRP | A | 201 | 75.003 | 56.700 | 70.038 | 1.00 | 37.10 | 6 |
| ATOM | 473 | C | TRP | A | 201 | 70.097 | 56.952 | 64.882 | 1.00 | 34.33 | 6 |
| ATOM | 474 | O | TRP | A | 201 | 69.510 | 56.080 | 65.517 | 1.00 | 33.11 | 8 |
| ATOM | 475 | N | VAL | A | 202 | 70.559 | 56.775 | 63.647 | 1.00 | 36.85 | 7 |
| ATOM | 476 | CA | VAL | A | 202 | 70.367 | 55.529 | 62.913 | 1.00 | 38.96 | 6 |
| ATOM | 477 | CB | VAL | A | 202 | 69.350 | 55.750 | 61.767 | 1.00 | 40.01 | 6 |
| ATOM | 478 | CG1 | VAL | A | 202 | 69.085 | 54.449 | 61.033 | 1.00 | 43.41 | 6 |
| ATOM | 479 | CG2 | VAL | A | 202 | 68.055 | 56.316 | 62.328 | 1.00 | 40.66 | 6 |
| ATOM | 480 | C | VAL | A | 202 | 71.664 | 54.993 | 62.314 | 1.00 | 39.12 | 6 |
| ATOM | 481 | O | VAL | A | 202 | 72.680 | 55.690 | 62.268 | 1.00 | 37.87 | 8 |
| ATOM | 482 | N | THR | A | 203 | 71.613 | 53.747 | 61.856 | 1.00 | 38.84 | 7 |
| ATOM | 483 | CA | THR | A | 203 | 72.754 | 53.082 | 61.239 | 1.00 | 40.24 | 6 |
| ATOM | 484 | CB | THR | A | 203 | 73.549 | 52.242 | 62.276 | 1.00 | 39.40 | 6 |
| ATOM | 485 | OG1 | THR | A | 203 | 74.603 | 51.531 | 61.616 | 1.00 | 38.31 | 8 |
| ATOM | 486 | CG2 | THR | A | 203 | 72.636 | 51.249 | 62.976 | 1.00 | 41.20 | 6 |
| ATOM | 487 | C | THR | A | 203 | 72.254 | 52.161 | 60.124 | 1.00 | 40.15 | 6 |
| ATOM | 488 | O | THR | A | 203 | 71.137 | 51.646 | 60.188 | 1.00 | 40.78 | 8 |
| ATOM | 489 | N | SER | A | 204 | 73.081 | 51.969 | 59.101 | 1.00 | 40.08 | 7 |
| ATOM | 490 | CA | SER | A | 204 | 72.728 | 51.110 | 57.972 | 1.00 | 41.48 | 6 |
| ATOM | 491 | CB | SER | A | 204 | 73.236 | 51.712 | 56.665 | 1.00 | 40.66 | 6 |
| ATOM | 492 | OG | SER | A | 204 | 72.486 | 52.859 | 56.328 | 1.00 | 46.94 | 8 |
| ATOM | 493 | C | SER | A | 204 | 73.317 | 49.722 | 58.133 | 1.00 | 39.55 | 6 |
| ATOM | 494 | O | SER | A | 204 | 73.119 | 48.857 | 57.285 | 1.00 | 41.01 | 8 |
| ATOM | 495 | N | LYS | A | 205 | 74.051 | 49.522 | 59.219 | 1.00 | 38.82 | 7 |
| ATOM | 496 | CA | LYS | A | 205 | 74.681 | 48.242 | 59.493 | 1.00 | 37.56 | 6 |
| ATOM | 497 | CB | LYS | A | 205 | 75.671 | 48.394 | 60.645 | 1.00 | 40.15 | 6 |
| ATOM | 498 | CG | LYS | A | 205 | 76.658 | 49.543 | 60.463 | 1.00 | 42.40 | 6 |
| ATOM | 499 | CD | LYS | A | 205 | 77.596 | 49.663 | 61.662 | 1.00 | 46.90 | 6 |
| ATOM | 500 | CE | LYS | A | 205 | 78.471 | 50.916 | 61.582 | 1.00 | 47.18 | 6 |
| ATOM | 501 | NZ | LYS | A | 205 | 77.679 | 52.178 | 61.696 | 1.00 | 46.85 | 7 |
| ATOM | 502 | C | LYS | A | 205 | 73.644 | 47.175 | 59.837 | 1.00 | 37.13 | 6 |
| ATOM | 503 | O | LYS | A | 205 | 72.578 | 47.474 | 60.389 | 1.00 | 32.51 | 8 |
| ATOM | 504 | N | PRO | A | 206 | 73.943 | 45.908 | 59.516 | 1.00 | 37.22 | 7 |
| ATOM | 505 | CD | PRO | A | 206 | 75.141 | 45.368 | 58.852 | 1.00 | 37.51 | 6 |
| ATOM | 506 | CA | PRO | A | 206 | 72.990 | 44.840 | 59.820 | 1.00 | 39.41 | 6 |
| ATOM | 507 | CB | PRO | A | 206 | 73.560 | 43.652 | 59.054 | 1.00 | 39.48 | 6 |
| ATOM | 508 | CG | PRO | A | 206 | 75.033 | 43.886 | 59.144 | 1.00 | 37.54 | 6 |
| ATOM | 509 | C | PRO | A | 206 | 72.915 | 44.587 | 61.319 | 1.00 | 40.78 | 6 |
| ATOM | 510 | O | PRO | A | 206 | 73.866 | 44.881 | 62.049 | 1.00 | 39.86 | 8 |
| ATOM | 511 | N | LEU | A | 207 | 71.782 | 44.063 | 61.777 | 1.00 | 42.43 | 7 |
| ATOM | 512 | CA | LEU | A | 207 | 71.614 | 43.759 | 63.192 | 1.00 | 44.83 | 6 |
| ATOM | 513 | CB | LEU | A | 207 | 70.167 | 43.360 | 63.503 | 1.00 | 45.74 | 6 |
| ATOM | 514 | CG | LEU | A | 207 | 69.444 | 43.977 | 64.712 | 1.00 | 46.64 | 6 |
| ATOM | 515 | CD1 | LEU | A | 207 | 68.219 | 43.127 | 65.034 | 1.00 | 47.31 | 6 |
| ATOM | 516 | CD2 | LEU | A | 207 | 70.355 | 44.039 | 65.923 | 1.00 | 46.33 | 6 |
| ATOM | 517 | C | LEU | A | 207 | 72.527 | 42.572 | 63.466 | 1.00 | 45.91 | 6 |
| ATOM | 518 | O | LEU | A | 207 | 72.424 | 41.536 | 62.805 | 1.00 | 44.13 | 8 |
| ATOM | 519 | N | PRO | A | 208 | 73.446 | 42.713 | 64.433 | 1.00 | 47.69 | 7 |
| ATOM | 520 | CD | PRO | A | 208 | 73.684 | 43.874 | 65.308 | 1.00 | 48.31 | 6 |
| ATOM | 521 | CA | PRO | A | 208 | 74.360 | 41.615 | 64.756 | 1.00 | 48.38 | 6 |
| ATOM | 522 | CB | PRO | A | 208 | 75.243 | 42.210 | 65.855 | 1.00 | 49.10 | 6 |
| ATOM | 523 | CG | PRO | A | 208 | 74.358 | 43.241 | 66.496 | 1.00 | 48.65 | 6 |
| ATOM | 524 | C | PRO | A | 208 | 73.626 | 40.352 | 65.202 | 1.00 | 49.69 | 6 |
| ATOM | 525 | O | PRO | A | 208 | 72.506 | 40.420 | 65.719 | 1.00 | 48.02 | 8 |
| ATOM | 526 | N | GLU | A | 209 | 74.263 | 39.203 | 64.989 | 1.00 | 51.42 | 7 |
| ATOM | 527 | CA | GLU | A | 209 | 73.683 | 37.918 | 65.359 | 1.00 | 54.40 | 6 |
| ATOM | 528 | CB | GLU | A | 209 | 74.666 | 36.787 | 65.045 | 1.00 | 56.31 | 6 |
| ATOM | 529 | CG | GLU | A | 209 | 74.951 | 36.592 | 63.562 | 1.00 | 59.24 | 6 |
| ATOM | 530 | CD | GLU | A | 209 | 75.847 | 35.394 | 63.293 | 1.00 | 60.98 | 6 |
| ATOM | 531 | OE1 | GLU | A | 209 | 75.487 | 34.274 | 63.720 | 1.00 | 62.38 | 8 |
| ATOM | 532 | OE2 | GLU | A | 209 | 76.908 | 35.571 | 62.655 | 1.00 | 61.16 | 8 |
| ATOM | 533 | C | GLU | A | 209 | 73.290 | 37.844 | 66.833 | 1.00 | 54.96 | 6 |
| ATOM | 534 | O | GLU | A | 209 | 72.205 | 37.368 | 67.170 | 1.00 | 54.11 | 8 |
| ATOM | 535 | N | TYR | A | 210 | 74.173 | 38.324 | 67.704 | 1.00 | 55.27 | 7 |
| ATOM | 536 | CA | TYR | A | 210 | 73.921 | 38.285 | 69.138 | 1.00 | 55.72 | 6 |
| ATOM | 537 | CB | TYR | A | 210 | 75.199 | 38.615 | 69.909 | 1.00 | 54.97 | 6 |
| ATOM | 538 | CG | TYR | A | 210 | 75.837 | 39.934 | 69.545 | 1.00 | 53.50 | 6 |
| ATOM | 539 | CD1 | TYR | A | 210 | 75.246 | 41.146 | 69.902 | 1.00 | 52.23 | 6 |
| ATOM | 540 | CE1 | TYR | A | 210 | 75.852 | 42.359 | 69.576 | 1.00 | 51.04 | 6 |
| ATOM | 541 | CD2 | TYR | A | 210 | 77.043 | 39.968 | 68.852 | 1.00 | 52.93 | 6 |
| ATOM | 542 | CE2 | TYR | A | 210 | 77.650 | 41.167 | 68.521 | 1.00 | 51.58 | 6 |
| ATOM | 543 | CZ | TYR | A | 210 | 77.054 | 42.356 | 68.885 | 1.00 | 50.20 | 6 |
| ATOM | 544 | OH | TYR | A | 210 | 77.670 | 43.537 | 68.547 | 1.00 | 49.63 | 8 |
| ATOM | 545 | C | TYR | A | 210 | 72.786 | 39.173 | 69.617 | 1.00 | 57.31 | 6 |
| ATOM | 546 | O | TYR | A | 210 | 72.507 | 39.227 | 70.812 | 1.00 | 58.22 | 8 |
| ATOM | 547 | N | LEU | A | 211 | 72.136 | 39.878 | 68.699 | 1.00 | 58.25 | 7 |
| ATOM | 548 | CA | LEU | A | 211 | 71.010 | 40.715 | 69.081 | 1.00 | 59.76 | 6 |
| ATOM | 549 | CB | LEU | A | 211 | 71.127 | 42.120 | 68.480 | 1.00 | 58.89 | 6 |
| ATOM | 550 | CG | LEU | A | 211 | 72.043 | 43.077 | 69.252 | 1.00 | 58.32 | 6 |
| ATOM | 551 | CD1 | LEU | A | 211 | 71.811 | 44.503 | 68.782 | 1.00 | 59.00 | 6 |
| ATOM | 552 | CD2 | LEU | A | 211 | 71.755 | 42.981 | 70.740 | 1.00 | 57.43 | 6 |
| ATOM | 553 | C | LEU | A | 211 | 69.705 | 40.055 | 68.650 | 1.00 | 61.58 | 6 |
| ATOM | 554 | O | LEU | A | 211 | 68.665 | 40.244 | 69.287 | 1.00 | 61.40 | 8 |
| ATOM | 555 | N | TRP | A | 212 | 69.797 | 39.276 | 67.573 | 1.00 | 63.39 | 7 |
| ATOM | 556 | CA | TRP | A | 212 | 68.622 | 38.564 | 67.087 | 1.00 | 65.26 | 6 |
| ATOM | 557 | CB | TRP | A | 212 | 68.908 | 37.881 | 65.748 | 1.00 | 64.31 | 6 |
| ATOM | 558 | CG | TRP | A | 212 | 68.828 | 38.786 | 64.552 | 1.00 | 63.93 | 6 |
| ATOM | 559 | CD2 | TRP | A | 212 | 67.659 | 39.382 | 63.970 | 1.00 | 63.86 | 6 |
| ATOM | 560 | CE2 | TRP | A | 212 | 68.080 | 40.100 | 62.829 | 1.00 | 63.73 | 6 |
| ATOM | 561 | CE3 | TRP | A | 212 | 66.296 | 39.375 | 64.300 | 1.00 | 63.75 | 6 |
| ATOM | 562 | CD1 | TRP | A | 212 | 69.879 | 39.162 | 63.767 | 1.00 | 63.25 | 6 |
| ATOM | 563 | NE1 | TRP | A | 212 | 69.438 | 39.950 | 62.729 | 1.00 | 63.95 | 7 |
| ATOM | 564 | CZ2 | TRP | A | 212 | 67.186 | 40.806 | 62.015 | 1.00 | 63.72 | 6 |
| ATOM | 565 | CZ3 | TRP | A | 212 | 65.406 | 40.078 | 63.488 | 1.00 | 63.47 | 6 |
| ATOM | 566 | CH2 | TRP | A | 212 | 65.857 | 40.783 | 62.359 | 1.00 | 63.40 | 6 |
| ATOM | 567 | C | TRP | A | 212 | 68.156 | 37.503 | 68.116 | 1.00 | 67.11 | 6 |
| ATOM | 568 | O | TRP | A | 212 | 66.974 | 37.411 | 68.406 | 1.00 | 67.04 | 8 |
| ATOM | 569 | N | LYS | A | 213 | 69.071 | 36.687 | 68.684 | 1.00 | 69.60 | 7 |
| ATOM | 570 | CA | LYS | A | 213 | 68.615 | 35.692 | 69.657 | 1.00 | 72.45 | 6 |
| ATOM | 571 | CB | LYS | A | 213 | 69.795 | 35.083 | 70.434 | 1.00 | 72.91 | 6 |
| ATOM | 572 | CG | LYS | A | 213 | 71.151 | 35.178 | 69.720 | 1.00 | 73.41 | 6 |
| ATOM | 573 | CD | LYS | A | 213 | 72.265 | 34.534 | 70.547 | 1.00 | 74.50 | 6 |
| ATOM | 574 | CE | LYS | A | 213 | 73.571 | 34.451 | 69.782 | 1.00 | 74.02 | 6 |
| ATOM | 575 | NZ | LYS | A | 213 | 74.610 | 33.685 | 70.527 | 1.00 | 74.26 | 7 |
| ATOM | 576 | C | LYS | A | 213 | 67.622 | 36.344 | 70.646 | 1.00 | 73.83 | 6 |
| ATOM | 577 | O | LYS | A | 213 | 67.038 | 35.716 | 71.535 | 1.00 | 74.79 | 8 |
| ATOM | 578 | N | LYS | A | 214 | 67.504 | 37.656 | 70.401 | 1.00 | 74.78 | 7 |
| ATOM | 579 | CA | LYS | A | 214 | 66.777 | 38.671 | 71.168 | 1.00 | 75.80 | 6 |
| ATOM | 580 | CB | LYS | A | 214 | 67.666 | 39.911 | 71.249 | 1.00 | 76.17 | 6 |
| ATOM | 581 | CG | LYS | A | 214 | 67.624 | 40.622 | 72.596 | 1.00 | 76.65 | 6 |
| ATOM | 582 | CD | LYS | A | 214 | 68.876 | 40.438 | 73.455 | 1.00 | 77.27 | 6 |
| ATOM | 583 | CE | LYS | A | 214 | 68.722 | 41.169 | 74.789 | 1.00 | 77.92 | 6 |
| ATOM | 584 | NZ | LYS | A | 214 | 69.856 | 42.099 | 75.032 | 1.00 | 77.36 | 7 |
| ATOM | 585 | C | LYS | A | 214 | 65.424 | 39.034 | 70.615 | 1.00 | 76.65 | 6 |
| ATOM | 586 | O | LYS | A | 214 | 64.431 | 39.088 | 71.336 | 1.00 | 76.72 | 8 |
| ATOM | 587 | N | ILE | A | 215 | 65.404 | 39.272 | 69.299 | 1.00 | 53.16 | 7 |
| ATOM | 588 | CA | ILE | A | 215 | 64.168 | 39.541 | 68.633 | 1.00 | 53.16 | 6 |
| ATOM | 589 | CB | ILE | A | 215 | 64.360 | 40.201 | 67.242 | 1.00 | 80.75 | 6 |
| ATOM | 590 | CG2 | ILE | A | 215 | 63.026 | 40.546 | 66.618 | 1.00 | 81.62 | 6 |
| ATOM | 591 | CG1 | ILE | A | 215 | 65.234 | 41.449 | 67.375 | 1.00 | 80.02 | 6 |
| ATOM | 592 | CD1 | ILE | A | 215 | 64.621 | 42.693 | 66.755 | 1.00 | 53.16 | 6 |
| ATOM | 593 | C | ILE | A | 215 | 63.509 | 38.190 | 68.511 | 1.00 | 53.16 | 6 |
| ATOM | 594 | O | ILE | A | 215 | 64.121 | 37.253 | 68.018 | 1.00 | 53.16 | 8 |
| ATOM | 595 | N | ALA | A | 216 | 62.338 | 37.988 | 69.040 | 1.00 | 169.91 | 7 |
| ATOM | 596 | CA | ALA | A | 216 | 61.822 | 36.612 | 69.010 | 1.00 | 170.88 | 6 |
| ATOM | 597 | CB | ALA | A | 216 | 60.943 | 36.337 | 70.214 | 1.00 | 117.32 | 6 |
| ATOM | 598 | C | ALA | A | 216 | 61.069 | 36.378 | 67.730 | 1.00 | 171.23 | 6 |
| ATOM | 599 | O | ALA | A | 216 | 61.535 | 36.689 | 66.641 | 1.00 | 171.61 | 8 |
| ATOM | 600 | N | ASN | A | 217 | 59.879 | 35.833 | 67.882 | 1.00 | 91.06 | 7 |
| ATOM | 601 | CA | ASN | A | 217 | 59.083 | 35.804 | 66.679 | 1.00 | 90.58 | 6 |
| ATOM | 602 | CB | ASN | A | 217 | 57.590 | 35.479 | 66.957 | 1.00 | 96.28 | 6 |
| ATOM | 603 | CG | ASN | A | 217 | 57.130 | 34.159 | 66.328 | 1.00 | 96.73 | 6 |
| ATOM | 604 | OD1 | ASN | A | 217 | 57.642 | 33.759 | 65.285 | 1.00 | 96.34 | 8 |
| ATOM | 605 | ND2 | ASN | A | 217 | 56.163 | 33.497 | 66.963 | 1.00 | 97.05 | 7 |
| ATOM | 606 | C | ASN | A | 217 | 59.252 | 37.263 | 66.204 | 1.00 | 90.98 | 6 |
| ATOM | 607 | O | ASN | A | 217 | 59.471 | 38.146 | 67.033 | 1.00 | 91.44 | 8 |
| ATOM | 608 | N | ASN | A | 218 | 59.175 | 37.549 | 64.915 | 1.00 | 53.16 | 7 |
| ATOM | 609 | CA | ASN | A | 218 | 59.400 | 38.896 | 64.425 | 1.00 | 53.16 | 6 |
| ATOM | 610 | CB | ASN | A | 218 | 58.601 | 39.086 | 63.147 | 1.00 | 92.18 | 6 |
| ATOM | 611 | CG | ASN | A | 218 | 59.490 | 39.810 | 62.161 | 1.00 | 92.63 | 6 |
| ATOM | 612 | OD1 | ASN | A | 218 | 59.313 | 41.001 | 61.880 | 1.00 | 94.15 | 8 |
| ATOM | 613 | ND2 | ASN | A | 218 | 60.460 | 39.089 | 61.616 | 1.00 | 93.35 | 7 |
| ATOM | 614 | C | ASN | A | 218 | 59.165 | 40.079 | 65.362 | 1.00 | 53.16 | 6 |
| ATOM | 615 | O | ASN | A | 218 | 59.884 | 41.074 | 65.328 | 1.00 | 53.16 | 8 |
| ATOM | 616 | N | CYS | A | 219 | 58.144 | 39.953 | 66.190 | 1.00 | 70.98 | 7 |
| ATOM | 617 | CA | CYS | A | 219 | 57.667 | 40.983 | 67.144 | 1.00 | 70.17 | 6 |
| ATOM | 618 | CB | CYS | A | 219 | 56.550 | 40.386 | 68.019 | 1.00 | 132.50 | 6 |
| ATOM | 619 | SG | CYS | A | 219 | 57.144 | 39.614 | 69.557 | 1.00 | 138.89 | 16 |
| ATOM | 620 | C | CYS | A | 219 | 58.679 | 41.623 | 68.105 | 1.00 | 68.14 | 6 |
| ATOM | 621 | O | CYS | A | 219 | 59.373 | 40.961 | 68.866 | 1.00 | 67.32 | 8 |
| ATOM | 622 | N | ILE | A | 220 | 58.709 | 42.949 | 68.042 | 1.00 | 53.16 | 7 |
| ATOM | 623 | CA | ILE | A | 220 | 59.562 | 43.695 | 69.014 | 1.00 | 53.16 | 6 |
| ATOM | 624 | CB | ILE | A | 220 | 60.555 | 44.678 | 68.343 | 1.00 | 53.16 | 6 |
| ATOM | 625 | CG2 | ILE | A | 220 | 61.212 | 45.590 | 69.370 | 1.00 | 53.16 | 6 |
| ATOM | 626 | CG1 | ILE | A | 220 | 61.621 | 43.904 | 67.577 | 1.00 | 53.16 | 6 |
| ATOM | 627 | CD1 | ILE | A | 220 | 62.779 | 44.756 | 67.102 | 1.00 | 53.16 | 6 |
| ATOM | 628 | C | ILE | A | 220 | 58.660 | 44.470 | 69.915 | 1.00 | 53.16 | 6 |
| ATOM | 629 | O | ILE | A | 220 | 57.905 | 45.331 | 69.472 | 1.00 | 53.16 | 8 |
| ATOM | 630 | N | PHE | A | 221 | 58.777 | 44.312 | 71.214 | 1.00 | 53.16 | 7 |
| ATOM | 631 | CA | PHE | A | 221 | 57.907 | 45.054 | 72.121 | 1.00 | 53.16 | 6 |
| ATOM | 632 | CB | PHE | A | 221 | 57.840 | 44.180 | 73.398 | 1.00 | 124.59 | 6 |
| ATOM | 633 | CG | PHE | A | 221 | 57.154 | 42.817 | 73.251 | 1.00 | 126.22 | 6 |
| ATOM | 634 | CD1 | PHE | A | 221 | 55.807 | 42.771 | 72.864 | 1.00 | 127.67 | 6 |
| ATOM | 635 | CD2 | PHE | A | 221 | 57.792 | 41.616 | 73.511 | 1.00 | 126.30 | 6 |
| ATOM | 636 | CE1 | PHE | A | 221 | 55.134 | 41.579 | 72.769 | 1.00 | 128.32 | 6 |
| ATOM | 637 | CE2 | PHE | A | 221 | 57.125 | 40.411 | 73.421 | 1.00 | 126.64 | 6 |
| ATOM | 638 | CZ | PHE | A | 221 | 55.780 | 40.407 | 73.062 | 1.00 | 126.98 | 6 |
| ATOM | 639 | C | PHE | A | 221 | 58.270 | 46.582 | 72.404 | 1.00 | 53.16 | 6 |
| ATOM | 640 | O | PHE | A | 221 | 59.406 | 46.877 | 72.751 | 1.00 | 53.16 | 8 |
| ATOM | 641 | N | ILE | A | 222 | 57.296 | 47.537 | 72.275 | 1.00 | 64.18 | 7 |
| ATOM | 642 | CA | ILE | A | 222 | 57.527 | 48.977 | 72.591 | 1.00 | 63.35 | 6 |
| ATOM | 643 | CB | ILE | A | 222 | 57.687 | 49.872 | 71.360 | 1.00 | 62.62 | 6 |
| ATOM | 644 | CG2 | ILE | A | 222 | 57.824 | 51.334 | 71.762 | 1.00 | 63.22 | 6 |
| ATOM | 645 | CG1 | ILE | A | 222 | 58.895 | 49.449 | 70.528 | 1.00 | 63.17 | 6 |
| ATOM | 646 | CD1 | ILE | A | 222 | 59.350 | 50.496 | 69.533 | 1.00 | 63.67 | 6 |
| ATOM | 647 | C | ILE | A | 222 | 56.389 | 49.591 | 73.382 | 1.00 | 63.29 | 6 |
| ATOM | 648 | O | ILE | A | 222 | 55.250 | 49.597 | 72.942 | 1.00 | 63.66 | 8 |
| ATOM | 649 | N | LYS | A | 223 | 56.724 | 50.106 | 74.566 | 1.00 | 62.68 | 7 |
| ATOM | 650 | CA | LYS | A | 223 | 55.767 | 50.701 | 75.477 | 1.00 | 61.51 | 6 |
| ATOM | 651 | C | LYS | A | 223 | 55.561 | 52.184 | 75.171 | 1.00 | 61.43 | 6 |
| ATOM | 652 | O | LYS | A | 223 | 56.486 | 52.986 | 75.271 | 1.00 | 60.49 | 8 |
| ATOM | 653 | CB | LYS | A | 223 | 56.191 | 50.472 | 76.934 | 1.00 | 61.52 | 6 |
| ATOM | 654 | CG | LYS | A | 223 | 56.257 | 49.000 | 77.312 | 1.00 | 20.00 | 6 |
| ATOM | 655 | CD | LYS | A | 223 | 56.630 | 48.818 | 78.776 | 1.00 | 20.00 | 6 |
| ATOM | 656 | CE | LYS | A | 223 | 56.336 | 47.411 | 79.256 | 1.00 | 20.00 | 6 |
| ATOM | 657 | NZ | LYS | A | 223 | 56.692 | 47.216 | 80.692 | 1.00 | 20.00 | 7 |
| ATOM | 658 | N | ILE | A | 224 | 54.343 | 52.475 | 74.785 | 1.00 | 61.21 | 7 |
| ATOM | 659 | CA | ILE | A | 224 | 53.980 | 53.836 | 74.483 | 1.00 | 61.68 | 6 |
| ATOM | 660 | CB | ILE | A | 224 | 53.124 | 53.900 | 73.226 | 1.00 | 61.28 | 6 |
| ATOM | 661 | CG2 | ILE | A | 224 | 52.807 | 55.328 | 72.841 | 1.00 | 60.55 | 6 |
| ATOM | 662 | CG1 | ILE | A | 224 | 53.843 | 53.185 | 72.098 | 1.00 | 61.68 | 6 |
| ATOM | 663 | CD1 | ILE | A | 224 | 53.077 | 53.192 | 70.790 | 1.00 | 61.94 | 6 |
| ATOM | 664 | C | ILE | A | 224 | 53.302 | 54.497 | 75.675 | 1.00 | 62.84 | 6 |
| ATOM | 665 | O | ILE | A | 224 | 52.370 | 53.959 | 76.259 | 1.00 | 63.59 | 8 |
| ATOM | 666 | N | HIS | A | 225 | 53.810 | 55.666 | 76.003 | 1.00 | 62.46 | 7 |
| ATOM | 667 | CA | HIS | A | 225 | 53.317 | 56.449 | 77.081 | 1.00 | 63.71 | 6 |
| ATOM | 668 | CB | HIS | A | 225 | 54.424 | 56.828 | 78.041 | 1.00 | 60.91 | 6 |
| ATOM | 669 | CG | HIS | A | 225 | 55.144 | 55.689 | 78.771 | 1.00 | 59.21 | 6 |
| ATOM | 670 | CD2 | HIS | A | 225 | 55.295 | 55.498 | 80.107 | 1.00 | 58.46 | 6 |
| ATOM | 671 | ND1 | HIS | A | 225 | 55.833 | 54.649 | 78.182 | 1.00 | 58.01 | 7 |
| ATOM | 672 | CE1 | HIS | A | 225 | 56.345 | 53.866 | 79.121 | 1.00 | 56.19 | 6 |
| ATOM | 673 | NE2 | HIS | A | 225 | 56.033 | 54.377 | 80.297 | 1.00 | 56.41 | 7 |
| ATOM | 674 | C | HIS | A | 225 | 52.915 | 57.789 | 76.571 | 1.00 | 65.64 | 6 |
| ATOM | 675 | O | HIS | A | 225 | 53.732 | 58.470 | 75.943 | 1.00 | 65.89 | 8 |
| ATOM | 676 | N | ARG | A | 226 | 51.711 | 58.180 | 76.825 | 1.00 | 68.03 | 7 |
| ATOM | 677 | CA | ARG | A | 226 | 51.375 | 59.527 | 76.547 | 1.00 | 70.15 | 6 |
| ATOM | 678 | CB | ARG | A | 226 | 49.902 | 59.709 | 76.234 | 1.00 | 70.72 | 6 |
| ATOM | 679 | CG | ARG | A | 226 | 49.451 | 61.148 | 76.148 | 1.00 | 71.54 | 6 |
| ATOM | 680 | CD | ARG | A | 226 | 50.433 | 61.894 | 75.294 | 1.00 | 72.11 | 6 |
| ATOM | 681 | NE | ARG | A | 226 | 50.061 | 63.273 | 75.164 | 1.00 | 72.14 | 7 |
| ATOM | 682 | CZ | ARG | A | 226 | 50.533 | 64.052 | 74.195 | 1.00 | 72.31 | 6 |
| ATOM | 683 | NH1 | ARG | A | 226 | 51.372 | 63.557 | 73.295 | 1.00 | 71.85 | 7 |
| ATOM | 684 | NH2 | ARG | A | 226 | 50.166 | 65.324 | 74.130 | 1.00 | 72.33 | 7 |
| ATOM | 685 | C | ARG | A | 226 | 51.695 | 60.103 | 77.866 | 1.00 | 71.32 | 6 |
| ATOM | 686 | O | ARG | A | 226 | 52.855 | 60.351 | 78.208 | 1.00 | 71.29 | 8 |
| ATOM | 687 | N | SER | A | 227 | 50.634 | 60.311 | 78.629 | 1.00 | 62.53 | 7 |
| ATOM | 688 | CA | SER | A | 227 | 50.974 | 60.710 | 79.967 | 1.00 | 64.13 | 6 |
| ATOM | 689 | CB | SER | A | 227 | 49.596 | 61.366 | 79.992 | 1.00 | 53.16 | 6 |
| ATOM | 690 | OG | SER | A | 227 | 49.340 | 61.994 | 81.234 | 1.00 | 53.16 | 8 |
| ATOM | 691 | C | SER | A | 227 | 51.142 | 59.742 | 81.096 | 1.00 | 64.53 | 6 |
| ATOM | 692 | O | SER | A | 227 | 52.147 | 59.037 | 81.220 | 1.00 | 66.11 | 8 |
| ATOM | 693 | N | THR | A | 228 | 50.127 | 59.706 | 81.913 | 1.00 | 121.38 | 7 |
| ATOM | 694 | CA | THR | A | 228 | 50.193 | 58.802 | 82.991 | 1.00 | 120.74 | 6 |
| ATOM | 695 | CB | THR | A | 228 | 49.150 | 59.125 | 84.047 | 1.00 | 92.83 | 6 |
| ATOM | 696 | OG1 | THR | A | 228 | 49.792 | 59.477 | 85.275 | 1.00 | 92.58 | 8 |
| ATOM | 697 | CG2 | THR | A | 228 | 48.231 | 57.926 | 84.258 | 1.00 | 92.58 | 6 |
| ATOM | 698 | C | THR | A | 228 | 49.976 | 57.402 | 82.439 | 1.00 | 120.59 | 6 |
| ATOM | 699 | O | THR | A | 228 | 50.293 | 56.402 | 83.079 | 1.00 | 120.30 | 8 |
| ATOM | 700 | N | THR | A | 229 | 49.425 | 57.343 | 81.229 | 1.00 | 84.64 | 7 |
| ATOM | 701 | CA | THR | A | 229 | 49.104 | 56.081 | 80.575 | 1.00 | 84.19 | 6 |
| ATOM | 702 | CB | THR | A | 229 | 47.948 | 56.271 | 79.580 | 1.00 | 81.94 | 6 |
| ATOM | 703 | OG1 | THR | A | 229 | 46.819 | 56.838 | 80.259 | 1.00 | 82.50 | 8 |
| ATOM | 704 | CG2 | THR | A | 229 | 47.538 | 54.941 | 78.972 | 1.00 | 81.21 | 6 |
| ATOM | 705 | C | THR | A | 229 | 50.289 | 55.447 | 79.844 | 1.00 | 83.50 | 6 |
| ATOM | 706 | O | THR | A | 229 | 51.187 | 56.137 | 79.371 | 1.00 | 83.04 | 8 |
| ATOM | 707 | N | SER | A | 230 | 50.262 | 54.123 | 79.754 | 1.00 | 53.16 | 7 |
| ATOM | 708 | CA | SER | A | 230 | 51.310 | 53.346 | 79.101 | 1.00 | 53.16 | 6 |
| ATOM | 709 | CB | SER | A | 230 | 52.357 | 52.922 | 80.140 | 1.00 | 53.16 | 6 |
| ATOM | 710 | OG | SER | A | 230 | 52.750 | 51.562 | 79.994 | 1.00 | 53.16 | 8 |
| ATOM | 711 | C | SER | A | 230 | 50.691 | 52.107 | 78.454 | 1.00 | 53.16 | 6 |
| ATOM | 712 | O | SER | A | 230 | 49.986 | 51.341 | 79.113 | 1.00 | 53.16 | 8 |
| ATOM | 713 | N | GLN | A | 231 | 50.946 | 51.916 | 77.162 | 1.00 | 53.16 | 7 |
| ATOM | 714 | CA | GLN | A | 231 | 50.414 | 50.765 | 76.438 | 1.00 | 53.16 | 6 |
| ATOM | 715 | CB | GLN | A | 231 | 49.521 | 51.221 | 75.281 | 1.00 | 53.16 | 6 |
| ATOM | 716 | CG | GLN | A | 231 | 48.241 | 51.912 | 75.690 | 1.00 | 53.16 | 6 |
| ATOM | 717 | CD | GLN | A | 231 | 47.446 | 51.115 | 76.708 | 1.00 | 53.16 | 6 |
| ATOM | 718 | OE1 | GLN | A | 231 | 47.753 | 51.139 | 77.897 | 1.00 | 53.16 | 8 |
| ATOM | 719 | NE2 | GLN | A | 231 | 46.425 | 50.402 | 76.245 | 1.00 | 53.16 | 7 |
| ATOM | 720 | C | GLN | A | 231 | 51.554 | 49.920 | 75.879 | 1.00 | 53.16 | 6 |
| ATOM | 721 | O | GLN | A | 231 | 52.649 | 50.428 | 75.659 | 1.00 | 53.16 | 8 |
| ATOM | 722 | N | THR | A | 232 | 51.295 | 48.634 | 75.651 | 1.00 | 53.16 | 7 |
| ATOM | 723 | CA | THR | A | 232 | 52.299 | 47.740 | 75.091 | 1.00 | 53.16 | 6 |
| ATOM | 724 | CB | THR | A | 232 | 52.481 | 46.480 | 75.928 | 1.00 | 53.16 | 6 |
| ATOM | 725 | OG1 | THR | A | 232 | 53.037 | 46.821 | 77.202 | 1.00 | 53.16 | 8 |
| ATOM | 726 | CG2 | THR | A | 232 | 53.408 | 45.493 | 75.220 | 1.00 | 53.16 | 6 |
| ATOM | 727 | C | THR | A | 232 | 51.875 | 47.300 | 73.701 | 1.00 | 53.16 | 6 |
| ATOM | 728 | O | THR | A | 232 | 50.908 | 46.556 | 73.552 | 1.00 | 53.16 | 8 |
| ATOM | 729 | N | ILE | A | 233 | 52.603 | 47.753 | 72.688 | 1.00 | 53.16 | 7 |
| ATOM | 730 | CA | ILE | A | 233 | 52.291 | 47.371 | 71.309 | 1.00 | 53.16 | 6 |
| ATOM | 731 | CB | ILE | A | 233 | 52.186 | 48.638 | 70.407 | 1.00 | 58.58 | 6 |
| ATOM | 732 | CG2 | ILE | A | 233 | 51.945 | 48.248 | 68.965 | 1.00 | 59.33 | 6 |
| ATOM | 733 | CG1 | ILE | A | 233 | 51.071 | 49.560 | 70.899 | 1.00 | 59.50 | 6 |
| ATOM | 734 | CD1 | ILE | A | 233 | 51.041 | 50.904 | 70.208 | 1.00 | 53.16 | 6 |
| ATOM | 735 | C | ILE | A | 233 | 53.343 | 46.425 | 70.763 | 1.00 | 53.16 | 6 |
| ATOM | 736 | O | ILE | A | 233 | 54.537 | 46.696 | 70.812 | 1.00 | 53.16 | 8 |
| ATOM | 737 | N | LYS | A | 234 | 52.863 | 45.312 | 70.244 | 1.00 | 53.16 | 7 |
| ATOM | 738 | CA | LYS | A | 234 | 53.700 | 44.308 | 69.621 | 1.00 | 53.16 | 6 |
| ATOM | 739 | CB | LYS | A | 234 | 52.937 | 42.991 | 69.423 | 1.00 | 153.67 | 6 |
| ATOM | 740 | CG | LYS | A | 234 | 53.812 | 41.802 | 69.054 | 1.00 | 154.03 | 6 |
| ATOM | 741 | CD | LYS | A | 234 | 53.043 | 40.486 | 69.163 | 1.00 | 153.67 | 6 |
| ATOM | 742 | CE | LYS | A | 234 | 51.748 | 40.495 | 68.360 | 1.00 | 151.85 | 6 |
| ATOM | 743 | NZ | LYS | A | 234 | 50.929 | 39.268 | 68.584 | 1.00 | 148.73 | 7 |
| ATOM | 744 | C | LYS | A | 234 | 54.139 | 44.871 | 68.260 | 1.00 | 53.16 | 6 |
| ATOM | 745 | O | LYS | A | 234 | 53.341 | 44.959 | 67.332 | 1.00 | 53.16 | 8 |
| ATOM | 746 | N | VAL | A | 235 | 55.409 | 45.239 | 68.124 | 1.00 | 53.16 | 7 |
| ATOM | 747 | CA | VAL | A | 235 | 55.919 | 45.799 | 66.875 | 1.00 | 53.16 | 6 |
| ATOM | 748 | CB | VAL | A | 235 | 56.650 | 47.137 | 67.151 | 1.00 | 65.81 | 6 |
| ATOM | 749 | CG1 | VAL | A | 235 | 57.350 | 47.641 | 65.897 | 1.00 | 66.09 | 6 |
| ATOM | 750 | CG2 | VAL | A | 235 | 55.659 | 48.165 | 67.663 | 1.00 | 66.37 | 6 |
| ATOM | 751 | C | VAL | A | 235 | 56.867 | 44.827 | 66.163 | 1.00 | 53.16 | 6 |
| ATOM | 752 | O | VAL | A | 235 | 57.549 | 44.034 | 66.810 | 1.00 | 53.16 | 8 |
| ATOM | 753 | N | SER | A | 236 | 56.895 | 44.913 | 64.828 | 1.00 | 53.16 | 7 |
| ATOM | 754 | CA | SER | A | 236 | 57.790 | 44.092 | 63.963 | 1.00 | 53.16 | 6 |
| ATOM | 755 | CB | SER | A | 236 | 57.070 | 43.625 | 62.697 | 1.00 | 115.18 | 6 |
| ATOM | 756 | OG | SER | A | 236 | 57.927 | 42.848 | 61.879 | 1.00 | 113.12 | 8 |
| ATOM | 757 | C | SER | A | 236 | 59.047 | 44.874 | 63.630 | 1.00 | 53.16 | 6 |
| ATOM | 758 | O | SER | A | 236 | 58.964 | 46.085 | 63.480 | 1.00 | 53.16 | 8 |
| ATOM | 759 | N | PRO | A | 237 | 60.223 | 44.222 | 63.506 | 1.00 | 53.16 | 7 |
| ATOM | 760 | CD | PRO | A | 237 | 60.487 | 42.980 | 64.241 | 1.00 | 53.16 | 6 |
| ATOM | 761 | CA | PRO | A | 237 | 61.478 | 44.999 | 63.328 | 1.00 | 53.16 | 6 |
| ATOM | 762 | CB | PRO | A | 237 | 62.519 | 43.892 | 63.176 | 1.00 | 53.16 | 6 |
| ATOM | 763 | CG | PRO | A | 237 | 61.980 | 42.840 | 64.137 | 1.00 | 53.16 | 6 |
| ATOM | 764 | C | PRO | A | 237 | 61.363 | 46.153 | 62.358 | 1.00 | 53.16 | 6 |
| ATOM | 765 | O | PRO | A | 237 | 61.456 | 47.338 | 62.730 | 1.00 | 53.16 | 8 |
| ATOM | 766 | N | ASP | A | 238 | 61.164 | 45.795 | 61.122 | 1.00 | 100.58 | 7 |
| ATOM | 767 | CA | ASP | A | 238 | 61.080 | 46.782 | 60.063 | 1.00 | 102.15 | 6 |
| ATOM | 768 | CB | ASP | A | 238 | 61.036 | 46.079 | 58.742 | 1.00 | 155.58 | 6 |
| ATOM | 769 | CG | ASP | A | 238 | 61.774 | 44.779 | 58.877 | 1.00 | 157.20 | 6 |
| ATOM | 770 | OD1 | ASP | A | 238 | 62.858 | 44.780 | 59.496 | 1.00 | 158.13 | 8 |
| ATOM | 771 | OD2 | ASP | A | 238 | 61.278 | 43.759 | 58.352 | 1.00 | 156.13 | 8 |
| ATOM | 772 | C | ASP | A | 238 | 59.925 | 47.733 | 60.234 | 1.00 | 102.94 | 6 |
| ATOM | 773 | O | ASP | A | 238 | 59.843 | 48.719 | 59.502 | 1.00 | 101.97 | 8 |
| ATOM | 774 | N | ASP | A | 239 | 59.049 | 47.472 | 61.162 | 1.00 | 65.88 | 7 |
| ATOM | 775 | CA | ASP | A | 239 | 58.022 | 48.441 | 61.351 | 1.00 | 66.83 | 6 |
| ATOM | 776 | CB | ASP | A | 239 | 57.198 | 48.181 | 62.590 | 1.00 | 111.82 | 6 |
| ATOM | 777 | CG | ASP | A | 239 | 55.839 | 47.561 | 62.260 | 1.00 | 113.49 | 6 |
| ATOM | 778 | OD1 | ASP | A | 239 | 55.168 | 48.042 | 61.313 | 1.00 | 115.54 | 8 |
| ATOM | 779 | OD2 | ASP | A | 239 | 55.462 | 46.594 | 62.965 | 1.00 | 113.11 | 8 |
| ATOM | 780 | C | ASP | A | 239 | 58.750 | 49.788 | 61.364 | 1.00 | 67.46 | 6 |
| ATOM | 781 | O | ASP | A | 239 | 59.905 | 49.866 | 61.807 | 1.00 | 67.62 | 8 |
| ATOM | 782 | N | THR | A | 240 | 58.101 | 50.837 | 60.869 | 1.00 | 53.16 | 7 |
| ATOM | 783 | CA | THR | A | 240 | 58.733 | 52.143 | 60.709 | 1.00 | 53.16 | 6 |
| ATOM | 784 | CB | THR | A | 240 | 58.371 | 52.694 | 59.359 | 1.00 | 53.16 | 6 |
| ATOM | 785 | OG1 | THR | A | 240 | 59.402 | 52.381 | 58.413 | 1.00 | 53.16 | 8 |
| ATOM | 786 | CG2 | THR | A | 240 | 58.197 | 54.214 | 59.453 | 1.00 | 53.16 | 6 |
| ATOM | 787 | C | THR | A | 240 | 58.325 | 53.169 | 61.761 | 1.00 | 53.16 | 6 |
| ATOM | 788 | O | THR | A | 240 | 57.178 | 53.195 | 62.221 | 1.00 | 53.16 | 8 |
| ATOM | 789 | N | PRO | A | 241 | 59.293 | 54.026 | 62.161 | 1.00 | 74.34 | 7 |
| ATOM | 790 | CD | PRO | A | 241 | 60.562 | 53.484 | 62.624 | 1.00 | 164.98 | 6 |
| ATOM | 791 | CA | PRO | A | 241 | 58.925 | 55.110 | 63.095 | 1.00 | 74.73 | 6 |
| ATOM | 792 | CB | PRO | A | 241 | 60.232 | 55.793 | 63.407 | 1.00 | 165.88 | 6 |
| ATOM | 793 | CG | PRO | A | 241 | 61.155 | 54.605 | 63.438 | 1.00 | 165.59 | 6 |
| ATOM | 794 | C | PRO | A | 241 | 57.808 | 55.959 | 62.548 | 1.00 | 74.42 | 6 |
| ATOM | 795 | O | PRO | A | 241 | 57.985 | 57.166 | 62.367 | 1.00 | 74.77 | 8 |
| ATOM | 796 | N | GLY | A | 242 | 56.661 | 55.393 | 62.276 | 1.00 | 68.38 | 7 |
| ATOM | 797 | CA | GLY | A | 242 | 55.589 | 56.174 | 61.729 | 1.00 | 67.15 | 6 |
| ATOM | 798 | C | GLY | A | 242 | 54.524 | 55.190 | 61.371 | 1.00 | 66.64 | 6 |
| ATOM | 799 | O | GLY | A | 242 | 53.344 | 55.419 | 61.633 | 1.00 | 65.15 | 8 |
| ATOM | 800 | N | ALA | A | 243 | 54.948 | 54.089 | 60.762 | 1.00 | 86.81 | 7 |
| ATOM | 801 | CA | ALA | A | 243 | 54.034 | 53.020 | 60.440 | 1.00 | 86.33 | 6 |
| ATOM | 802 | CB | ALA | A | 243 | 54.733 | 51.967 | 59.623 | 1.00 | 149.66 | 6 |
| ATOM | 803 | C | ALA | A | 243 | 53.618 | 52.462 | 61.789 | 1.00 | 85.73 | 6 |
| ATOM | 804 | O | ALA | A | 243 | 52.541 | 51.893 | 61.932 | 1.00 | 85.52 | 8 |
| ATOM | 805 | N | ILE | A | 244 | 54.486 | 52.635 | 62.783 | 1.00 | 53.16 | 7 |
| ATOM | 806 | CA | ILE | A | 244 | 54.190 | 52.165 | 64.130 | 1.00 | 53.16 | 6 |
| ATOM | 807 | CB | ILE | A | 244 | 55.429 | 52.229 | 65.046 | 1.00 | 53.16 | 6 |
| ATOM | 808 | CG2 | ILE | A | 244 | 55.010 | 52.083 | 66.510 | 1.00 | 53.16 | 6 |
| ATOM | 809 | CG1 | ILE | A | 244 | 56.415 | 51.137 | 64.641 | 1.00 | 53.16 | 6 |
| ATOM | 810 | CD1 | ILE | A | 244 | 57.621 | 51.015 | 65.545 | 1.00 | 53.16 | 6 |
| ATOM | 811 | C | ILE | A | 244 | 53.087 | 53.014 | 64.739 | 1.00 | 53.16 | 6 |
| ATOM | 812 | O | ILE | A | 244 | 52.226 | 52.498 | 65.445 | 1.00 | 53.16 | 8 |
| ATOM | 813 | N | LEU | A | 245 | 53.110 | 54.314 | 64.453 | 1.00 | 78.57 | 7 |
| ATOM | 814 | CA | LEU | A | 245 | 52.093 | 55.221 | 64.973 | 1.00 | 79.43 | 6 |
| ATOM | 815 | CB | LEU | A | 245 | 52.455 | 56.675 | 64.674 | 1.00 | 53.16 | 6 |
| ATOM | 816 | CG | LEU | A | 245 | 53.461 | 57.352 | 65.612 | 1.00 | 53.16 | 6 |
| ATOM | 817 | CD1 | LEU | A | 245 | 53.857 | 58.706 | 65.043 | 1.00 | 53.16 | 6 |
| ATOM | 818 | CD2 | LEU | A | 245 | 52.850 | 57.518 | 66.996 | 1.00 | 53.16 | 6 |
| ATOM | 819 | C | LEU | A | 245 | 50.733 | 54.911 | 64.379 | 1.00 | 80.99 | 6 |
| ATOM | 820 | O | LEU | A | 245 | 49.728 | 54.899 | 65.086 | 1.00 | 81.22 | 8 |
| ATOM | 821 | N | GLN | A | 246 | 50.705 | 54.658 | 63.074 | 1.00 | 53.16 | 7 |
| ATOM | 822 | CA | GLN | A | 246 | 49.441 | 54.343 | 62.420 | 1.00 | 53.16 | 6 |
| ATOM | 823 | CB | GLN | A | 246 | 49.652 | 54.030 | 60.941 | 1.00 | 53.88 | 6 |
| ATOM | 824 | CG | GLN | A | 246 | 49.815 | 55.280 | 60.086 | 1.00 | 54.99 | 6 |
| ATOM | 825 | CD | GLN | A | 246 | 49.986 | 54.971 | 58.611 | 1.00 | 56.11 | 6 |
| ATOM | 826 | OE1 | GLN | A | 246 | 49.273 | 54.133 | 58.054 | 1.00 | 56.70 | 8 |
| ATOM | 827 | NE2 | GLN | A | 246 | 50.927 | 55.656 | 57.966 | 1.00 | 55.61 | 7 |
| ATOM | 828 | C | GLN | A | 246 | 48.810 | 53.165 | 63.124 | 1.00 | 53.16 | 6 |
| ATOM | 829 | O | GLN | A | 246 | 47.637 | 53.218 | 63.498 | 1.00 | 53.16 | 8 |
| ATOM | 830 | N | SER | A | 247 | 49.604 | 52.119 | 63.323 | 1.00 | 53.16 | 7 |
| ATOM | 831 | CA | SER | A | 247 | 49.123 | 50.919 | 63.996 | 1.00 | 53.16 | 6 |
| ATOM | 832 | CB | SER | A | 247 | 50.215 | 49.840 | 64.022 | 1.00 | 114.03 | 6 |
| ATOM | 833 | OG | SER | A | 247 | 51.328 | 50.258 | 64.782 | 1.00 | 114.92 | 8 |
| ATOM | 834 | C | SER | A | 247 | 48.684 | 51.234 | 65.419 | 1.00 | 53.16 | 6 |
| ATOM | 835 | O | SER | A | 247 | 47.872 | 50.512 | 65.990 | 1.00 | 53.16 | 8 |
| ATOM | 836 | N | PHE | A | 248 | 49.204 | 52.322 | 65.981 | 1.00 | 53.16 | 7 |
| ATOM | 837 | CA | PHE | A | 248 | 48.845 | 52.698 | 67.347 | 1.00 | 53.16 | 6 |
| ATOM | 838 | CB | PHE | A | 248 | 50.040 | 53.328 | 68.067 | 1.00 | 53.16 | 6 |
| ATOM | 839 | CG | PHE | A | 248 | 49.689 | 53.730 | 69.474 | 1.00 | 53.16 | 6 |
| ATOM | 840 | CD1 | PHE | A | 248 | 49.549 | 52.748 | 70.442 | 1.00 | 53.16 | 6 |
| ATOM | 841 | CD2 | PHE | A | 248 | 49.470 | 55.057 | 69.819 | 1.00 | 53.16 | 6 |
| ATOM | 842 | CE1 | PHE | A | 248 | 49.184 | 53.073 | 71.735 | 1.00 | 53.16 | 6 |
| ATOM | 843 | CE2 | PHE | A | 248 | 49.103 | 55.394 | 71.113 | 1.00 | 53.16 | 6 |
| ATOM | 844 | CZ | PHE | A | 248 | 48.963 | 54.395 | 72.072 | 1.00 | 53.16 | 6 |
| ATOM | 845 | C | PHE | A | 248 | 47.688 | 53.688 | 67.405 | 1.00 | 53.16 | 6 |
| ATOM | 846 | O | PHE | A | 248 | 47.189 | 53.984 | 68.483 | 1.00 | 53.16 | 8 |
| ATOM | 847 | N | PHE | A | 249 | 47.245 | 54.182 | 66.290 | 1.00 | 60.39 | 7 |
| ATOM | 848 | CA | PHE | A | 249 | 46.200 | 55.188 | 66.325 | 1.00 | 61.38 | 6 |
| ATOM | 849 | CB | PHE | A | 249 | 46.528 | 56.257 | 65.372 | 1.00 | 53.16 | 6 |
| ATOM | 850 | CG | PHE | A | 249 | 47.073 | 57.342 | 66.161 | 1.00 | 53.16 | 6 |
| ATOM | 851 | CD1 | PHE | A | 249 | 47.840 | 58.306 | 65.526 | 1.00 | 53.16 | 6 |
| ATOM | 852 | CD2 | PHE | A | 249 | 46.826 | 57.411 | 67.525 | 1.00 | 53.16 | 6 |
| ATOM | 853 | CE1 | PHE | A | 249 | 48.379 | 59.322 | 66.238 | 1.00 | 53.16 | 6 |
| ATOM | 854 | CE2 | PHE | A | 249 | 47.359 | 58.452 | 68.242 | 1.00 | 53.16 | 6 |
| ATOM | 855 | CZ | PHE | A | 249 | 48.149 | 59.407 | 67.600 | 1.00 | 53.16 | 6 |
| ATOM | 856 | C | PHE | A | 249 | 44.902 | 54.655 | 65.961 | 1.00 | 62.53 | 6 |
| ATOM | 857 | O | PHE | A | 249 | 43.835 | 55.234 | 66.208 | 1.00 | 63.10 | 8 |
| ATOM | 858 | N | THR | A | 250 | 44.996 | 53.506 | 65.358 | 1.00 | 53.16 | 7 |
| ATOM | 859 | CA | THR | A | 250 | 43.840 | 52.787 | 64.960 | 1.00 | 53.16 | 6 |
| ATOM | 860 | CB | THR | A | 250 | 44.154 | 51.844 | 63.817 | 1.00 | 158.58 | 6 |
| ATOM | 861 | OG1 | THR | A | 250 | 45.165 | 50.916 | 64.236 | 1.00 | 159.30 | 8 |
| ATOM | 862 | CG2 | THR | A | 250 | 44.625 | 52.621 | 62.592 | 1.00 | 158.25 | 6 |
| ATOM | 863 | C | THR | A | 250 | 43.356 | 52.062 | 66.189 | 1.00 | 53.16 | 6 |
| ATOM | 864 | O | THR | A | 250 | 42.141 | 51.945 | 66.436 | 1.00 | 53.16 | 8 |
| ATOM | 865 | N | LYS | A | 251 | 44.295 | 51.571 | 66.956 | 1.00 | 180.93 | 7 |
| ATOM | 866 | CA | LYS | A | 251 | 43.885 | 50.880 | 68.154 | 1.00 | 181.15 | 6 |
| ATOM | 867 | CB | LYS | A | 251 | 45.071 | 50.171 | 68.811 | 1.00 | 108.05 | 6 |
| ATOM | 868 | CG | LYS | A | 251 | 44.676 | 49.099 | 69.815 | 1.00 | 107.68 | 6 |
| ATOM | 869 | CD | LYS | A | 251 | 44.075 | 47.885 | 69.121 | 1.00 | 107.38 | 6 |
| ATOM | 870 | CE | LYS | A | 251 | 43.240 | 47.056 | 70.083 | 1.00 | 108.75 | 6 |
| ATOM | 871 | NZ | LYS | A | 251 | 43.397 | 45.596 | 69.838 | 1.00 | 108.71 | 7 |
| ATOM | 872 | C | LYS | A | 251 | 43.280 | 51.868 | 69.129 | 1.00 | 181.42 | 6 |
| ATOM | 873 | O | LYS | A | 251 | 42.121 | 51.777 | 69.552 | 1.00 | 182.21 | 8 |
| ATOM | 874 | N | MET | A | 252 | 44.108 | 52.817 | 69.466 | 1.00 | 87.91 | 7 |
| ATOM | 875 | CA | MET | A | 252 | 43.717 | 53.881 | 70.330 | 1.00 | 87.58 | 6 |
| ATOM | 876 | CB | MET | A | 252 | 44.960 | 54.609 | 70.833 | 1.00 | 67.67 | 6 |
| ATOM | 877 | CG | MET | A | 252 | 45.206 | 54.465 | 72.328 | 1.00 | 68.16 | 6 |
| ATOM | 878 | SD | MET | A | 252 | 45.636 | 52.774 | 72.808 | 1.00 | 70.52 | 16 |
| ATOM | 879 | CE | MET | A | 252 | 44.066 | 52.189 | 73.452 | 1.00 | 67.73 | 6 |
| ATOM | 880 | C | MET | A | 252 | 42.819 | 54.868 | 69.599 | 1.00 | 87.42 | 6 |
| ATOM | 881 | O | MET | A | 252 | 42.812 | 56.054 | 69.912 | 1.00 | 87.91 | 8 |
| ATOM | 882 | N | ALA | A | 253 | 42.053 | 54.379 | 68.635 | 1.00 | 172.74 | 7 |
| ATOM | 883 | CA | ALA | A | 253 | 41.171 | 55.251 | 67.875 | 1.00 | 172.74 | 6 |
| ATOM | 884 | CB | ALA | A | 253 | 40.640 | 54.551 | 66.631 | 1.00 | 123.46 | 6 |
| ATOM | 885 | C | ALA | A | 253 | 40.012 | 55.738 | 68.728 | 1.00 | 172.74 | 6 |
| ATOM | 886 | O | ALA | A | 253 | 39.680 | 56.919 | 68.731 | 1.00 | 172.74 | 8 |
| ATOM | 887 | N | LYS | A | 254 | 39.399 | 54.811 | 69.455 | 1.00 | 92.29 | 7 |
| ATOM | 888 | CA | LYS | A | 254 | 38.260 | 55.141 | 70.294 | 1.00 | 92.11 | 6 |
| ATOM | 889 | CB | LYS | A | 254 | 37.090 | 54.194 | 69.994 | 1.00 | 73.18 | 6 |
| ATOM | 890 | CG | LYS | A | 254 | 36.636 | 54.219 | 68.532 | 1.00 | 71.96 | 6 |
| ATOM | 891 | CD | LYS | A | 254 | 35.922 | 52.927 | 68.170 | 1.00 | 71.34 | 6 |
| ATOM | 892 | CE | LYS | A | 254 | 34.725 | 52.690 | 69.072 | 1.00 | 71.22 | 6 |
| ATOM | 893 | NZ | LYS | A | 254 | 34.107 | 51.358 | 68.821 | 1.00 | 70.34 | 7 |
| ATOM | 894 | C | LYS | A | 254 | 38.623 | 55.112 | 71.774 | 1.00 | 92.50 | 6 |
| ATOM | 895 | O | LYS | A | 254 | 37.756 | 54.993 | 72.643 | 1.00 | 92.80 | 8 |
| ATOM | 896 | N | LYS | A | 255 | 39.919 | 55.207 | 72.046 | 1.00 | 53.16 | 7 |
| ATOM | 897 | CA | LYS | A | 255 | 40.428 | 55.234 | 73.413 | 1.00 | 53.16 | 6 |
| ATOM | 898 | CB | LYS | A | 255 | 41.284 | 54.000 | 73.727 | 1.00 | 53.16 | 6 |
| ATOM | 899 | CG | LYS | A | 255 | 40.721 | 52.666 | 73.295 | 1.00 | 53.16 | 6 |
| ATOM | 900 | CD | LYS | A | 255 | 40.372 | 51.822 | 74.492 | 1.00 | 53.16 | 6 |
| ATOM | 901 | CE | LYS | A | 255 | 40.403 | 50.334 | 74.164 | 1.00 | 53.16 | 6 |
| ATOM | 902 | NZ | LYS | A | 255 | 41.787 | 49.779 | 74.221 | 1.00 | 53.16 | 7 |
| ATOM | 903 | C | LYS | A | 255 | 41.316 | 56.474 | 73.494 | 1.00 | 53.16 | 6 |
| ATOM | 904 | O | LYS | A | 255 | 42.066 | 56.651 | 74.455 | 1.00 | 53.16 | 8 |
| ATOM | 905 | N | LYS | A | 256 | 41.232 | 57.319 | 72.464 | 1.00 | 53.16 | 7 |
| ATOM | 906 | CA | LYS | A | 256 | 42.021 | 58.547 | 72.390 | 1.00 | 53.16 | 6 |
| ATOM | 907 | CB | LYS | A | 256 | 41.447 | 59.494 | 71.342 | 1.00 | 53.16 | 6 |
| ATOM | 908 | CG | LYS | A | 256 | 41.676 | 59.068 | 69.910 | 1.00 | 53.16 | 6 |
| ATOM | 909 | CD | LYS | A | 256 | 43.109 | 59.304 | 69.470 | 1.00 | 53.16 | 6 |
| ATOM | 910 | CE | LYS | A | 256 | 43.177 | 60.341 | 68.339 | 1.00 | 53.16 | 6 |
| ATOM | 911 | NZ | LYS | A | 256 | 42.430 | 59.965 | 67.094 | 1.00 | 53.16 | 7 |
| ATOM | 912 | C | LYS | A | 256 | 42.037 | 59.252 | 73.723 | 1.00 | 53.16 | 6 |
| ATOM | 913 | O | LYS | A | 256 | 43.026 | 59.878 | 74.095 | 1.00 | 53.16 | 8 |
| ATOM | 914 | N | SER | A | 257 | 40.927 | 59.175 | 74.443 | 1.00 | 53.16 | 7 |
| ATOM | 915 | CA | SER | A | 257 | 40.866 | 59.809 | 75.739 | 1.00 | 53.16 | 6 |
| ATOM | 916 | CB | SER | A | 257 | 39.502 | 59.580 | 76.379 | 1.00 | 53.16 | 6 |
| ATOM | 917 | OG | SER | A | 257 | 39.479 | 60.095 | 77.695 | 1.00 | 53.16 | 8 |
| ATOM | 918 | C | SER | A | 257 | 41.961 | 59.174 | 76.583 | 1.00 | 53.16 | 6 |
| ATOM | 919 | O | SER | A | 257 | 42.863 | 59.859 | 77.046 | 1.00 | 53.16 | 8 |
| ATOM | 920 | N | LEU | A | 258 | 41.889 | 57.858 | 76.743 | 1.00 | 53.16 | 7 |
| ATOM | 921 | CA | LEU | A | 258 | 42.867 | 57.115 | 77.524 | 1.00 | 53.16 | 6 |
| ATOM | 922 | CB | LEU | A | 258 | 42.796 | 55.635 | 77.165 | 1.00 | 53.16 | 6 |
| ATOM | 923 | CG | LEU | A | 258 | 41.960 | 54.814 | 78.139 | 1.00 | 53.16 | 6 |
| ATOM | 924 | CD1 | LEU | A | 258 | 41.899 | 53.379 | 77.679 | 1.00 | 53.16 | 6 |
| ATOM | 925 | CD2 | LEU | A | 258 | 42.572 | 54.923 | 79.527 | 1.00 | 53.16 | 6 |
| ATOM | 926 | C | LEU | A | 258 | 44.312 | 57.600 | 77.413 | 1.00 | 53.16 | 6 |
| ATOM | 927 | O | LEU | A | 258 | 45.032 | 57.613 | 78.412 | 1.00 | 53.16 | 8 |
| ATOM | 928 | N | MET | A | 259 | 44.743 | 57.980 | 76.211 | 1.00 | 70.41 | 7 |
| ATOM | 929 | CA | MET | A | 259 | 46.114 | 58.459 | 76.000 | 1.00 | 70.41 | 6 |
| ATOM | 930 | CB | MET | A | 259 | 46.663 | 57.947 | 74.657 | 1.00 | 53.16 | 6 |
| ATOM | 931 | CG | MET | A | 259 | 46.826 | 56.423 | 74.575 | 1.00 | 53.16 | 6 |
| ATOM | 932 | SD | MET | A | 259 | 47.979 | 55.757 | 75.793 | 1.00 | 53.16 | 16 |
| ATOM | 933 | CE | MET | A | 259 | 49.500 | 55.743 | 74.845 | 1.00 | 53.16 | 6 |
| ATOM | 934 | C | MET | A | 259 | 46.190 | 59.972 | 76.048 | 1.00 | 70.41 | 6 |
| ATOM | 935 | O | MET | A | 259 | 47.024 | 60.575 | 75.385 | 1.00 | 70.41 | 8 |
| ATOM | 936 | N | ASP | A | 260 | 45.315 | 60.576 | 76.838 | 1.00 | 53.16 | 7 |
| ATOM | 937 | CA | ASP | A | 260 | 45.276 | 62.020 | 76.986 | 1.00 | 53.16 | 6 |
| ATOM | 938 | CB | ASP | A | 260 | 46.281 | 62.462 | 78.056 | 1.00 | 86.22 | 6 |
| ATOM | 939 | CG | ASP | A | 260 | 46.150 | 63.927 | 78.406 | 1.00 | 86.22 | 6 |
| ATOM | 940 | OD1 | ASP | A | 260 | 45.017 | 64.363 | 78.687 | 1.00 | 86.22 | 8 |
| ATOM | 941 | OD2 | ASP | A | 260 | 47.169 | 64.647 | 78.404 | 1.00 | 86.22 | 8 |
| ATOM | 942 | C | ASP | A | 260 | 45.535 | 62.779 | 75.672 | 1.00 | 53.16 | 6 |
| ATOM | 943 | O | ASP | A | 260 | 46.038 | 63.890 | 75.670 | 1.00 | 53.16 | 8 |
| ATOM | 944 | N | ILE | A | 261 | 45.179 | 62.123 | 74.563 | 1.00 | 70.71 | 7 |
| ATOM | 945 | CA | ILE | A | 261 | 45.388 | 62.669 | 73.212 | 1.00 | 70.71 | 6 |
| ATOM | 946 | CB | ILE | A | 261 | 45.736 | 61.519 | 72.246 | 1.00 | 58.77 | 6 |
| ATOM | 947 | CG2 | ILE | A | 261 | 45.890 | 62.032 | 70.836 | 1.00 | 58.77 | 6 |
| ATOM | 948 | CG1 | ILE | A | 261 | 47.024 | 60.811 | 72.696 | 1.00 | 58.77 | 6 |
| ATOM | 949 | CD1 | ILE | A | 261 | 47.300 | 59.507 | 71.973 | 1.00 | 58.77 | 6 |
| ATOM | 950 | C | ILE | A | 261 | 44.182 | 63.383 | 72.642 | 1.00 | 70.71 | 6 |
| ATOM | 951 | O | ILE | A | 261 | 43.158 | 62.747 | 72.357 | 1.00 | 70.71 | 8 |
| ATOM | 952 | N | PRO | A | 262 | 44.248 | 64.720 | 72.472 | 1.00 | 94.39 | 7 |
| ATOM | 953 | CD | PRO | A | 262 | 44.677 | 65.532 | 73.619 | 1.00 | 89.42 | 6 |
| ATOM | 954 | CA | PRO | A | 262 | 43.062 | 65.465 | 71.858 | 1.00 | 94.39 | 6 |
| ATOM | 955 | CB | PRO | A | 262 | 43.490 | 66.901 | 71.981 | 1.00 | 89.42 | 6 |
| ATOM | 956 | CG | PRO | A | 262 | 44.189 | 66.916 | 73.321 | 1.00 | 89.42 | 6 |
| ATOM | 957 | C | PRO | A | 262 | 42.588 | 65.001 | 70.483 | 1.00 | 94.39 | 6 |
| ATOM | 958 | O | PRO | A | 262 | 43.405 | 64.811 | 69.590 | 1.00 | 94.39 | 8 |
| ATOM | 959 | N | GLU | A | 263 | 41.272 | 64.795 | 70.303 | 1.00 | 70.58 | 7 |
| ATOM | 960 | CA | GLU | A | 263 | 40.741 | 64.404 | 68.992 | 1.00 | 70.58 | 6 |
| ATOM | 961 | CB | GLU | A | 263 | 39.219 | 64.420 | 68.993 | 1.00 | 95.56 | 6 |
| ATOM | 962 | CG | GLU | A | 263 | 38.570 | 63.855 | 70.245 | 1.00 | 95.56 | 6 |
| ATOM | 963 | CD | GLU | A | 263 | 38.528 | 62.348 | 70.223 | 1.00 | 95.56 | 6 |
| ATOM | 964 | OE1 | GLU | A | 263 | 38.663 | 61.758 | 69.139 | 1.00 | 95.56 | 8 |
| ATOM | 965 | OE2 | GLU | A | 263 | 38.354 | 61.749 | 71.297 | 1.00 | 95.56 | 8 |
| ATOM | 966 | C | GLU | A | 263 | 41.266 | 65.369 | 67.929 | 1.00 | 70.58 | 6 |
| ATOM | 967 | O | GLU | A | 263 | 41.859 | 64.941 | 66.932 | 1.00 | 70.58 | 8 |
| ATOM | 968 | N | SER | A | 264 | 41.029 | 66.663 | 68.129 | 1.00 | 102.57 | 7 |
| ATOM | 969 | CA | SER | A | 264 | 41.445 | 67.661 | 67.174 | 1.00 | 102.57 | 6 |
| ATOM | 970 | CB | SER | A | 264 | 41.303 | 69.051 | 67.795 | 1.00 | 68.04 | 6 |
| ATOM | 971 | OG | SER | A | 264 | 39.960 | 69.498 | 67.733 | 1.00 | 68.04 | 8 |
| ATOM | 972 | C | SER | A | 264 | 42.878 | 67.424 | 66.667 | 1.00 | 102.57 | 6 |
| ATOM | 973 | O | SER | A | 264 | 43.139 | 67.552 | 65.481 | 1.00 | 102.57 | 8 |
| ATOM | 974 | N | GLN | A | 265 | 43.826 | 67.079 | 67.528 | 1.00 | 160.37 | 7 |
| ATOM | 975 | CA | GLN | A | 265 | 45.236 | 67.031 | 67.091 | 1.00 | 161.10 | 6 |
| ATOM | 976 | CB | GLN | A | 265 | 46.184 | 67.115 | 68.299 | 1.00 | 190.73 | 6 |
| ATOM | 977 | CG | GLN | A | 265 | 47.663 | 66.915 | 67.888 | 1.00 | 126.22 | 6 |
| ATOM | 978 | CD | GLN | A | 265 | 48.637 | 67.978 | 68.380 | 1.00 | 126.22 | 6 |
| ATOM | 979 | OE1 | GLN | A | 265 | 48.932 | 68.964 | 67.691 | 1.00 | 126.22 | 8 |
| ATOM | 980 | NE2 | GLN | A | 265 | 49.243 | 67.977 | 69.567 | 1.00 | 126.22 | 7 |
| ATOM | 981 | C | GLN | A | 265 | 45.717 | 65.885 | 66.174 | 1.00 | 161.75 | 6 |
| ATOM | 982 | O | GLN | A | 265 | 45.428 | 64.697 | 66.348 | 1.00 | 161.28 | 8 |
| ATOM | 983 | N | SER | A | 266 | 46.481 | 66.421 | 65.205 | 1.00 | 204.74 | 7 |
| ATOM | 984 | CA | SER | A | 266 | 47.205 | 65.811 | 64.110 | 1.00 | 204.74 | 6 |
| ATOM | 985 | CB | SER | A | 266 | 48.410 | 66.680 | 63.768 | 1.00 | 130.68 | 6 |
| ATOM | 986 | OG | SER | A | 266 | 49.254 | 66.836 | 64.896 | 1.00 | 130.37 | 8 |
| ATOM | 987 | C | SER | A | 266 | 47.728 | 64.448 | 64.391 | 1.00 | 204.74 | 6 |
| ATOM | 988 | O | SER | A | 266 | 48.889 | 64.327 | 64.802 | 1.00 | 204.74 | 8 |
| ATOM | 989 | N | GLU | A | 267 | 46.940 | 63.412 | 64.183 | 1.00 | 80.49 | 7 |
| ATOM | 990 | CA | GLU | A | 267 | 47.537 | 62.139 | 64.436 | 1.00 | 80.85 | 6 |
| ATOM | 991 | CB | GLU | A | 267 | 46.562 | 61.012 | 64.004 | 1.00 | 81.22 | 6 |
| ATOM | 992 | CG | GLU | A | 267 | 45.234 | 60.915 | 64.773 | 1.00 | 81.37 | 6 |
| ATOM | 993 | CD | GLU | A | 267 | 44.793 | 59.479 | 65.084 | 1.00 | 81.83 | 6 |
| ATOM | 994 | OE1 | GLU | A | 267 | 44.955 | 58.587 | 64.219 | 1.00 | 81.59 | 8 |
| ATOM | 995 | OE2 | GLU | A | 267 | 44.277 | 59.239 | 66.208 | 1.00 | 81.54 | 8 |
| ATOM | 996 | C | GLU | A | 267 | 48.899 | 62.037 | 63.721 | 1.00 | 80.71 | 6 |
| ATOM | 997 | O | GLU | A | 267 | 49.680 | 61.107 | 63.990 | 1.00 | 80.77 | 8 |
| ATOM | 998 | N | GLN | A | 268 | 49.193 | 62.974 | 62.817 | 1.00 | 80.11 | 7 |
| ATOM | 999 | CA | GLN | A | 268 | 50.456 | 62.978 | 62.072 | 1.00 | 79.66 | 6 |
| ATOM | 1000 | CB | GLN | A | 268 | 50.261 | 63.635 | 60.731 | 1.00 | 80.70 | 6 |
| ATOM | 1001 | CG | GLN | A | 268 | 49.596 | 62.784 | 59.694 | 1.00 | 81.84 | 6 |
| ATOM | 1002 | CD | GLN | A | 268 | 49.893 | 63.318 | 58.328 | 1.00 | 82.96 | 6 |
| ATOM | 1003 | OE1 | GLN | A | 268 | 51.053 | 63.479 | 57.938 | 1.00 | 84.17 | 8 |
| ATOM | 1004 | NE2 | GLN | A | 268 | 49.001 | 63.659 | 57.408 | 1.00 | 83.16 | 7 |
| ATOM | 1005 | C | GLN | A | 268 | 51.496 | 63.795 | 62.741 | 1.00 | 78.49 | 6 |
| ATOM | 1006 | O | GLN | A | 268 | 52.677 | 63.461 | 62.782 | 1.00 | 78.84 | 8 |
| ATOM | 1007 | N | ASP | A | 269 | 51.024 | 64.875 | 63.269 | 1.00 | 76.14 | 7 |
| ATOM | 1008 | CA | ASP | A | 269 | 51.981 | 65.681 | 63.862 | 1.00 | 73.43 | 6 |
| ATOM | 1009 | CB | ASP | A | 269 | 51.373 | 66.979 | 64.315 | 1.00 | 74.13 | 6 |
| ATOM | 1010 | CG | ASP | A | 269 | 52.417 | 68.048 | 64.076 | 1.00 | 74.74 | 6 |
| ATOM | 1011 | OD1 | ASP | A | 269 | 53.629 | 67.729 | 64.220 | 1.00 | 74.86 | 8 |
| ATOM | 1012 | OD2 | ASP | A | 269 | 52.048 | 69.201 | 63.751 | 1.00 | 74.73 | 8 |
| ATOM | 1013 | C | ASP | A | 269 | 52.729 | 64.999 | 64.974 | 1.00 | 71.26 | 6 |
| ATOM | 1014 | O | ASP | A | 269 | 53.845 | 65.386 | 65.272 | 1.00 | 71.35 | 8 |
| ATOM | 1015 | N | PHE | A | 270 | 52.132 | 63.998 | 65.610 | 1.00 | 68.36 | 7 |
| ATOM | 1016 | CA | PHE | A | 270 | 52.842 | 63.401 | 66.710 | 1.00 | 65.37 | 6 |
| ATOM | 1017 | CB | PHE | A | 270 | 51.947 | 62.398 | 67.453 | 1.00 | 66.66 | 6 |
| ATOM | 1018 | CG | PHE | A | 270 | 50.812 | 63.021 | 68.212 | 1.00 | 68.15 | 6 |
| ATOM | 1019 | CD1 | PHE | A | 270 | 49.713 | 62.244 | 68.574 | 1.00 | 68.82 | 6 |
| ATOM | 1020 | CD2 | PHE | A | 270 | 50.816 | 64.372 | 68.552 | 1.00 | 68.34 | 6 |
| ATOM | 1021 | CE1 | PHE | A | 270 | 48.638 | 62.802 | 69.264 | 1.00 | 68.27 | 6 |
| ATOM | 1022 | CE2 | PHE | A | 270 | 49.746 | 64.940 | 69.241 | 1.00 | 68.55 | 6 |
| ATOM | 1023 | CZ | PHE | A | 270 | 48.656 | 64.153 | 69.597 | 1.00 | 68.84 | 6 |
| ATOM | 1024 | C | PHE | A | 270 | 54.132 | 62.708 | 66.272 | 1.00 | 62.71 | 6 |
| ATOM | 1025 | O | PHE | A | 270 | 54.320 | 62.386 | 65.095 | 1.00 | 61.87 | 8 |
| ATOM | 1026 | N | VAL | A | 271 | 55.009 | 62.475 | 67.244 | 1.00 | 58.53 | 7 |
| ATOM | 1027 | CA | VAL | A | 271 | 56.285 | 61.808 | 67.014 | 1.00 | 53.95 | 6 |
| ATOM | 1028 | CB | VAL | A | 271 | 57.404 | 62.828 | 66.714 | 1.00 | 54.38 | 6 |
| ATOM | 1029 | CG1 | VAL | A | 271 | 57.447 | 63.897 | 67.792 | 1.00 | 53.06 | 6 |
| ATOM | 1030 | CG2 | VAL | A | 271 | 58.734 | 62.119 | 66.648 | 1.00 | 54.45 | 6 |
| ATOM | 1031 | C | VAL | A | 271 | 56.691 | 60.985 | 68.237 | 1.00 | 50.72 | 6 |
| ATOM | 1032 | O | VAL | A | 271 | 56.392 | 61.354 | 69.371 | 1.00 | 50.38 | 8 |
| ATOM | 1033 | N | LEU | A | 272 | 57.365 | 59.864 | 68.004 | 1.00 | 47.85 | 7 |
| ATOM | 1034 | CA | LEU | A | 272 | 57.819 | 59.017 | 69.101 | 1.00 | 44.84 | 6 |
| ATOM | 1035 | CB | LEU | A | 272 | 57.971 | 57.569 | 68.627 | 1.00 | 44.61 | 6 |
| ATOM | 1036 | CG | LEU | A | 272 | 56.682 | 56.807 | 68.306 | 1.00 | 43.88 | 6 |
| ATOM | 1037 | CD1 | LEU | A | 272 | 57.017 | 55.504 | 67.615 | 1.00 | 43.57 | 6 |
| ATOM | 1038 | CD2 | LEU | A | 272 | 55.897 | 56.555 | 69.588 | 1.00 | 42.73 | 6 |
| ATOM | 1039 | C | LEU | A | 272 | 59.158 | 59.523 | 69.647 | 1.00 | 44.26 | 6 |
| ATOM | 1040 | O | LEU | A | 272 | 60.149 | 59.607 | 68.917 | 1.00 | 41.38 | 8 |
| ATOM | 1041 | N | ARG | A | 273 | 59.176 | 59.873 | 70.928 | 1.00 | 42.50 | 7 |
| ATOM | 1042 | CA | ARG | A | 273 | 60.393 | 60.355 | 71.569 | 1.00 | 43.47 | 6 |
| ATOM | 1043 | CB | ARG | A | 273 | 60.203 | 61.791 | 72.053 | 1.00 | 44.05 | 6 |
| ATOM | 1044 | CG | ARG | A | 273 | 61.485 | 62.460 | 72.513 | 1.00 | 47.87 | 6 |
| ATOM | 1045 | CD | ARG | A | 273 | 61.175 | 63.590 | 73.463 | 1.00 | 50.27 | 6 |
| ATOM | 1046 | NE | ARG | A | 273 | 60.604 | 63.071 | 74.701 | 1.00 | 54.75 | 7 |
| ATOM | 1047 | CZ | ARG | A | 273 | 60.029 | 63.816 | 75.637 | 1.00 | 55.37 | 6 |
| ATOM | 1048 | NH1 | ARG | A | 273 | 59.942 | 65.128 | 75.482 | 1.00 | 55.33 | 7 |
| ATOM | 1049 | NH2 | ARG | A | 273 | 59.540 | 63.243 | 76.728 | 1.00 | 56.47 | 7 |
| ATOM | 1050 | C | ARG | A | 273 | 60.709 | 59.457 | 72.758 | 1.00 | 42.89 | 6 |
| ATOM | 1051 | O | ARG | A | 273 | 59.823 | 59.123 | 73.539 | 1.00 | 44.55 | 8 |
| ATOM | 1052 | N | VAL | A | 274 | 61.970 | 59.090 | 72.859 | 1.00 | 42.34 | 7 |
| ATOM | 1053 | CA | VAL | A | 274 | 62.418 | 58.232 | 73.932 | 1.00 | 41.88 | 6 |
| ATOM | 1054 | CB | VAL | A | 274 | 63.895 | 57.879 | 73.869 | 1.00 | 41.66 | 6 |
| ATOM | 1055 | CG1 | VAL | A | 274 | 64.286 | 57.021 | 75.053 | 1.00 | 40.47 | 6 |
| ATOM | 1056 | CG2 | VAL | A | 274 | 64.225 | 57.170 | 72.573 | 1.00 | 39.63 | 6 |
| ATOM | 1057 | C | VAL | A | 274 | 62.157 | 58.910 | 75.244 | 1.00 | 43.05 | 6 |
| ATOM | 1058 | O | VAL | A | 274 | 62.500 | 60.079 | 75.449 | 1.00 | 43.12 | 8 |
| ATOM | 1059 | N | CYS | A | 275 | 61.540 | 58.168 | 76.159 | 1.00 | 42.16 | 7 |
| ATOM | 1060 | CA | CYS | A | 275 | 61.182 | 58.697 | 77.478 | 1.00 | 42.64 | 6 |
| ATOM | 1061 | CB | CYS | A | 275 | 60.332 | 57.677 | 78.224 | 1.00 | 44.30 | 6 |
| ATOM | 1062 | SG | CYS | A | 275 | 58.933 | 57.020 | 77.262 | 1.00 | 47.39 | 16 |
| ATOM | 1063 | C | CYS | A | 275 | 62.418 | 59.149 | 78.325 | 1.00 | 40.81 | 6 |
| ATOM | 1064 | O | CYS | A | 275 | 63.347 | 58.390 | 78.563 | 1.00 | 39.56 | 8 |
| ATOM | 1065 | N | GLY | A | 276 | 62.377 | 60.401 | 78.749 | 1.00 | 40.36 | 7 |
| ATOM | 1066 | CA | GLY | A | 276 | 63.420 | 61.007 | 79.575 | 1.00 | 39.95 | 6 |
| ATOM | 1067 | C | GLY | A | 276 | 64.673 | 61.357 | 78.792 | 1.00 | 40.02 | 6 |
| ATOM | 1068 | O | GLY | A | 276 | 65.709 | 61.648 | 79.382 | 1.00 | 39.08 | 8 |
| ATOM | 1069 | N | ARG | A | 277 | 64.616 | 61.337 | 77.466 | 1.00 | 39.68 | 7 |
| ATOM | 1070 | CA | ARG | A | 277 | 65.768 | 61.654 | 76.630 | 1.00 | 39.22 | 6 |
| ATOM | 1071 | CB | ARG | A | 277 | 66.454 | 60.371 | 76.160 | 1.00 | 38.38 | 6 |
| ATOM | 1072 | CG | ARG | A | 277 | 67.282 | 59.681 | 77.234 | 1.00 | 40.38 | 6 |
| ATOM | 1073 | CD | ARG | A | 277 | 68.093 | 58.554 | 76.625 | 1.00 | 40.52 | 6 |
| ATOM | 1074 | NE | ARG | A | 277 | 69.370 | 58.361 | 77.304 | 1.00 | 40.00 | 7 |
| ATOM | 1075 | CZ | ARG | A | 277 | 70.483 | 59.028 | 77.012 | 1.00 | 41.64 | 6 |
| ATOM | 1076 | NH1 | ARG | A | 277 | 70.488 | 59.932 | 76.042 | 1.00 | 40.07 | 7 |
| ATOM | 1077 | NH2 | ARG | A | 277 | 71.591 | 58.807 | 77.708 | 1.00 | 40.48 | 7 |
| ATOM | 1078 | C | ARG | A | 277 | 65.366 | 62.435 | 75.411 | 1.00 | 38.17 | 6 |
| ATOM | 1079 | O | ARG | A | 277 | 64.262 | 62.246 | 74.878 | 1.00 | 38.28 | 8 |
| ATOM | 1080 | N | ASP | A | 278 | 66.226 | 63.283 | 74.952 | 1.00 | 37.36 | 7 |
| ATOM | 1081 | CA | ASP | A | 278 | 65.881 | 63.981 | 73.768 | 1.00 | 36.42 | 6 |
| ATOM | 1082 | CB | ASP | A | 278 | 66.308 | 65.420 | 73.858 | 1.00 | 39.00 | 6 |
| ATOM | 1083 | CG | ASP | A | 278 | 65.410 | 66.155 | 74.818 | 1.00 | 42.30 | 6 |
| ATOM | 1084 | OD1 | ASP | A | 278 | 64.339 | 65.620 | 75.192 | 1.00 | 44.85 | 8 |
| ATOM | 1085 | OD2 | ASP | A | 278 | 65.766 | 67.290 | 75.194 | 1.00 | 46.12 | 8 |
| ATOM | 1086 | C | ASP | A | 278 | 66.468 | 63.275 | 72.568 | 1.00 | 35.36 | 6 |
| ATOM | 1087 | O | ASP | A | 278 | 67.387 | 63.766 | 71.923 | 1.00 | 34.32 | 8 |
| ATOM | 1088 | N | GLU | A | 279 | 65.916 | 62.091 | 72.305 | 1.00 | 34.50 | 7 |
| ATOM | 1089 | CA | GLU | A | 279 | 66.262 | 61.216 | 71.206 | 1.00 | 34.76 | 6 |
| ATOM | 1090 | CB | GLU | A | 279 | 66.870 | 59.893 | 71.722 | 1.00 | 33.94 | 6 |
| ATOM | 1091 | CG | GLU | A | 279 | 68.073 | 59.342 | 70.977 | 1.00 | 37.49 | 6 |
| ATOM | 1092 | CD | GLU | A | 279 | 68.351 | 60.086 | 69.691 | 1.00 | 40.49 | 6 |
| ATOM | 1093 | OE1 | GLU | A | 279 | 67.717 | 59.750 | 68.662 | 1.00 | 38.56 | 8 |
| ATOM | 1094 | OE2 | GLU | A | 279 | 69.197 | 61.004 | 69.699 | 1.00 | 41.09 | 8 |
| ATOM | 1095 | C | GLU | A | 279 | 64.968 | 60.917 | 70.485 | 1.00 | 34.06 | 6 |
| ATOM | 1096 | O | GLU | A | 279 | 63.935 | 60.673 | 71.128 | 1.00 | 33.71 | 8 |
| ATOM | 1097 | N | TYR | A | 280 | 64.976 | 60.942 | 69.181 | 1.00 | 33.18 | 7 |
| ATOM | 1098 | CA | TYR | A | 280 | 63.713 | 60.696 | 68.473 | 1.00 | 33.28 | 6 |
| ATOM | 1099 | CB | TYR | A | 280 | 63.303 | 61.944 | 67.676 | 1.00 | 32.81 | 6 |
| ATOM | 1100 | CG | TYR | A | 280 | 62.972 | 63.145 | 68.511 | 1.00 | 32.69 | 6 |
| ATOM | 1101 | CD1 | TYR | A | 280 | 63.947 | 64.038 | 68.946 | 1.00 | 33.03 | 6 |
| ATOM | 1102 | CE1 | TYR | A | 280 | 63.613 | 65.154 | 69.708 | 1.00 | 33.47 | 6 |
| ATOM | 1103 | CD2 | TYR | A | 280 | 61.648 | 63.394 | 68.865 | 1.00 | 34.08 | 6 |
| ATOM | 1104 | CE2 | TYR | A | 280 | 61.299 | 64.507 | 69.631 | 1.00 | 33.65 | 6 |
| ATOM | 1105 | CZ | TYR | A | 280 | 62.282 | 65.383 | 70.046 | 1.00 | 33.08 | 6 |
| ATOM | 1106 | OH | TYR | A | 280 | 61.939 | 66.497 | 70.779 | 1.00 | 31.24 | 8 |
| ATOM | 1107 | C | TYR | A | 280 | 63.814 | 59.540 | 67.488 | 1.00 | 34.74 | 6 |
| ATOM | 1108 | O | TYR | A | 280 | 64.790 | 59.413 | 66.749 | 1.00 | 33.01 | 8 |
| ATOM | 1109 | N | LEU | A | 281 | 62.781 | 58.708 | 67.476 | 1.00 | 36.65 | 7 |
| ATOM | 1110 | CA | LEU | A | 281 | 62.699 | 57.578 | 66.561 | 1.00 | 39.88 | 6 |
| ATOM | 1111 | CB | LEU | A | 281 | 62.079 | 56.369 | 67.273 | 1.00 | 39.41 | 6 |
| ATOM | 1112 | CG | LEU | A | 281 | 62.991 | 55.495 | 68.144 | 1.00 | 40.83 | 6 |
| ATOM | 1113 | CD1 | LEU | A | 281 | 63.950 | 56.356 | 68.937 | 1.00 | 39.99 | 6 |
| ATOM | 1114 | CD2 | LEU | A | 281 | 62.137 | 54.619 | 69.065 | 1.00 | 39.72 | 6 |
| ATOM | 1115 | C | LEU | A | 281 | 61.796 | 58.045 | 65.420 | 1.00 | 40.27 | 6 |
| ATOM | 1116 | O | LEU | A | 281 | 60.581 | 57.865 | 65.475 | 1.00 | 42.08 | 8 |
| ATOM | 1117 | N | VAL | A | 282 | 62.394 | 58.673 | 64.409 | 1.00 | 42.06 | 7 |
| ATOM | 1118 | CA | VAL | A | 282 | 61.658 | 59.195 | 63.253 | 1.00 | 42.12 | 6 |
| ATOM | 1119 | CB | VAL | A | 282 | 61.428 | 60.722 | 63.353 | 1.00 | 42.25 | 6 |
| ATOM | 1120 | CG1 | VAL | A | 282 | 60.639 | 61.046 | 64.575 | 1.00 | 42.55 | 6 |
| ATOM | 1121 | CG2 | VAL | A | 282 | 62.767 | 61.458 | 63.379 | 1.00 | 40.56 | 6 |
| ATOM | 1122 | C | VAL | A | 282 | 62.413 | 58.958 | 61.955 | 1.00 | 43.62 | 6 |
| ATOM | 1123 | O | VAL | A | 282 | 63.556 | 58.503 | 61.966 | 1.00 | 45.05 | 8 |
| ATOM | 1124 | N | GLY | A | 283 | 61.768 | 59.295 | 60.840 | 1.00 | 44.62 | 7 |
| ATOM | 1125 | CA | GLY | A | 283 | 62.377 | 59.122 | 59.533 | 1.00 | 44.64 | 6 |
| ATOM | 1126 | C | GLY | A | 283 | 62.015 | 57.793 | 58.905 | 1.00 | 45.32 | 6 |
| ATOM | 1127 | O | GLY | A | 283 | 61.517 | 56.898 | 59.589 | 1.00 | 44.71 | 8 |
| ATOM | 1128 | N | GLU | A | 284 | 62.254 | 57.669 | 57.599 | 1.00 | 46.10 | 7 |
| ATOM | 1129 | CA | GLU | A | 284 | 61.971 | 56.430 | 56.866 | 1.00 | 47.03 | 6 |
| ATOM | 1130 | CB | GLU | A | 284 | 61.955 | 56.683 | 55.354 | 1.00 | 49.05 | 6 |
| ATOM | 1131 | CG | GLU | A | 284 | 60.899 | 57.658 | 54.873 | 1.00 | 51.36 | 6 |
| ATOM | 1132 | CD | GLU | A | 284 | 59.524 | 57.321 | 55.402 | 1.00 | 54.24 | 6 |
| ATOM | 1133 | OE1 | GLU | A | 284 | 59.141 | 56.130 | 55.358 | 1.00 | 56.01 | 8 |
| ATOM | 1134 | OE2 | GLU | A | 284 | 58.824 | 58.249 | 55.860 | 1.00 | 54.92 | 8 |
| ATOM | 1135 | C | GLU | A | 284 | 63.064 | 55.413 | 57.180 | 1.00 | 46.09 | 6 |
| ATOM | 1136 | O | GLU | A | 284 | 63.938 | 55.154 | 56.352 | 1.00 | 45.83 | 8 |
| ATOM | 1137 | N | THR | A | 285 | 63.011 | 54.841 | 58.376 | 1.00 | 44.86 | 7 |
| ATOM | 1138 | CA | THR | A | 285 | 64.015 | 53.872 | 58.800 | 1.00 | 43.90 | 6 |
| ATOM | 1139 | CB | THR | A | 285 | 65.075 | 54.526 | 59.712 | 1.00 | 41.58 | 6 |
| ATOM | 1140 | OG1 | THR | A | 285 | 65.645 | 55.663 | 59.062 | 1.00 | 40.03 | 8 |
| ATOM | 1141 | CG2 | THR | A | 285 | 66.160 | 53.503 | 60.046 | 1.00 | 41.20 | 6 |
| ATOM | 1142 | C | THR | A | 285 | 63.402 | 52.744 | 59.609 | 1.00 | 43.48 | 6 |
| ATOM | 1143 | O | THR | A | 285 | 62.503 | 52.965 | 60.416 | 1.00 | 43.85 | 8 |
| ATOM | 1144 | N | PRO | A | 286 | 63.915 | 51.545 | 59.376 | 1.00 | 44.37 | 7 |
| ATOM | 1145 | CD | PRO | A | 286 | 63.948 | 51.035 | 58.017 | 1.00 | 42.72 | 6 |
| ATOM | 1146 | CA | PRO | A | 286 | 63.478 | 50.406 | 60.228 | 1.00 | 45.32 | 6 |
| ATOM | 1147 | CB | PRO | A | 286 | 64.215 | 49.225 | 59.621 | 1.00 | 44.93 | 6 |
| ATOM | 1148 | CG | PRO | A | 286 | 64.145 | 49.557 | 58.161 | 1.00 | 43.50 | 6 |
| ATOM | 1149 | C | PRO | A | 286 | 63.692 | 50.628 | 61.703 | 1.00 | 46.39 | 6 |
| ATOM | 1150 | O | PRO | A | 286 | 64.668 | 51.291 | 62.078 | 1.00 | 46.08 | 8 |
| ATOM | 1151 | N | ILE | A | 287 | 62.843 | 50.114 | 62.578 | 1.00 | 45.93 | 7 |
| ATOM | 1152 | CA | ILE | A | 287 | 63.083 | 50.352 | 63.996 | 1.00 | 46.28 | 6 |
| ATOM | 1153 | CB | ILE | A | 287 | 61.917 | 49.846 | 64.865 | 1.00 | 48.30 | 6 |
| ATOM | 1154 | CG2 | ILE | A | 287 | 61.816 | 48.330 | 64.790 | 1.00 | 50.48 | 6 |
| ATOM | 1155 | CG1 | ILE | A | 287 | 62.091 | 50.310 | 66.313 | 1.00 | 49.83 | 6 |
| ATOM | 1156 | CD1 | ILE | A | 287 | 60.884 | 50.051 | 67.187 | 1.00 | 53.01 | 6 |
| ATOM | 1157 | C | ILE | A | 287 | 64.371 | 49.685 | 64.462 | 1.00 | 44.46 | 6 |
| ATOM | 1158 | O | ILE | A | 287 | 65.080 | 50.222 | 65.302 | 1.00 | 44.38 | 8 |
| ATOM | 1159 | N | LYS | A | 288 | 64.652 | 48.506 | 63.918 | 1.00 | 42.78 | 7 |
| ATOM | 1160 | CA | LYS | A | 288 | 65.853 | 47.778 | 64.305 | 1.00 | 41.72 | 6 |
| ATOM | 1161 | CB | LYS | A | 288 | 65.871 | 46.396 | 63.660 | 1.00 | 41.81 | 6 |
| ATOM | 1162 | CG | LYS | A | 288 | 65.721 | 46.417 | 62.153 | 1.00 | 41.25 | 6 |
| ATOM | 1163 | CD | LYS | A | 288 | 65.938 | 45.027 | 61.571 | 1.00 | 43.20 | 6 |
| ATOM | 1164 | CE | LYS | A | 288 | 65.909 | 45.062 | 60.049 | 1.00 | 45.14 | 6 |
| ATOM | 1165 | NZ | LYS | A | 288 | 66.198 | 43.730 | 59.450 | 1.00 | 48.71 | 7 |
| ATOM | 1166 | C | LYS | A | 288 | 67.125 | 48.522 | 63.909 | 1.00 | 41.30 | 6 |
| ATOM | 1167 | O | LYS | A | 288 | 68.220 | 48.123 | 64.291 | 1.00 | 41.37 | 8 |
| ATOM | 1168 | N | ASN | A | 289 | 66.976 | 49.590 | 63.130 | 1.00 | 40.14 | 7 |
| ATOM | 1169 | CA | ASN | A | 289 | 68.124 | 50.371 | 62.689 | 1.00 | 39.86 | 6 |
| ATOM | 1170 | CB | ASN | A | 289 | 67.995 | 50.713 | 61.202 | 1.00 | 40.93 | 6 |
| ATOM | 1171 | CG | ASN | A | 289 | 68.413 | 49.559 | 60.313 | 1.00 | 41.80 | 6 |
| ATOM | 1172 | OD1 | ASN | A | 289 | 68.649 | 48.449 | 60.794 | 1.00 | 41.87 | 8 |
| ATOM | 1173 | ND2 | ASN | A | 289 | 68.507 | 49.812 | 59.014 | 1.00 | 43.28 | 7 |
| ATOM | 1174 | C | ASN | A | 289 | 68.348 | 51.632 | 63.505 | 1.00 | 39.26 | 6 |
| ATOM | 1175 | O | ASN | A | 289 | 69.151 | 52.484 | 63.133 | 1.00 | 40.41 | 8 |
| ATOM | 1176 | N | PHE | A | 290 | 67.639 | 51.754 | 64.619 | 1.00 | 37.80 | 7 |
| ATOM | 1177 | CA | PHE | A | 290 | 67.826 | 52.899 | 65.490 | 1.00 | 38.62 | 6 |
| ATOM | 1178 | CB | PHE | A | 290 | 66.497 | 53.384 | 66.070 | 1.00 | 38.47 | 6 |
| ATOM | 1179 | CG | PHE | A | 290 | 65.724 | 54.279 | 65.140 | 1.00 | 39.34 | 6 |
| ATOM | 1180 | CD1 | PHE | A | 290 | 64.924 | 53.742 | 64.136 | 1.00 | 37.04 | 6 |
| ATOM | 1181 | CD2 | PHE | A | 290 | 65.809 | 55.666 | 65.261 | 1.00 | 38.75 | 6 |
| ATOM | 1182 | CE1 | PHE | A | 290 | 64.218 | 54.570 | 63.271 | 1.00 | 37.47 | 6 |
| ATOM | 1183 | CE2 | PHE | A | 290 | 65.106 | 56.505 | 64.396 | 1.00 | 39.72 | 6 |
| ATOM | 1184 | CZ | PHE | A | 290 | 64.308 | 55.955 | 63.397 | 1.00 | 38.28 | 6 |
| ATOM | 1185 | C | PHE | A | 290 | 68.764 | 52.448 | 66.597 | 1.00 | 38.83 | 6 |
| ATOM | 1186 | O | PHE | A | 290 | 68.576 | 51.384 | 67.190 | 1.00 | 37.19 | 8 |
| ATOM | 1187 | N | GLN | A | 291 | 69.789 | 53.249 | 66.858 | 1.00 | 38.72 | 7 |
| ATOM | 1188 | CA | GLN | A | 291 | 70.768 | 52.905 | 67.880 | 1.00 | 40.85 | 6 |
| ATOM | 1189 | CB | GLN | A | 291 | 71.880 | 53.953 | 67.922 | 1.00 | 42.17 | 6 |
| ATOM | 1190 | CG | GLN | A | 291 | 73.116 | 53.520 | 67.160 | 1.00 | 47.34 | 6 |
| ATOM | 1191 | CD | GLN | A | 291 | 73.516 | 52.096 | 67.505 | 1.00 | 49.93 | 6 |
| ATOM | 1192 | OE1 | GLN | A | 291 | 73.022 | 51.137 | 66.909 | 1.00 | 50.65 | 8 |
| ATOM | 1193 | NE2 | GLN | A | 291 | 74.397 | 51.951 | 68.490 | 1.00 | 51.43 | 7 |
| ATOM | 1194 | C | GLN | A | 291 | 70.202 | 52.692 | 69.277 | 1.00 | 37.98 | 6 |
| ATOM | 1195 | O | GLN | A | 291 | 70.640 | 51.791 | 69.985 | 1.00 | 38.50 | 8 |
| ATOM | 1196 | N | TRP | A | 292 | 69.233 | 53.509 | 69.673 | 1.00 | 36.46 | 7 |
| ATOM | 1197 | CA | TRP | A | 292 | 68.633 | 53.363 | 70.997 | 1.00 | 37.72 | 6 |
| ATOM | 1198 | CB | TRP | A | 292 | 67.615 | 54.475 | 71.270 | 1.00 | 38.01 | 6 |
| ATOM | 1199 | CG | TRP | A | 292 | 67.055 | 54.394 | 72.658 | 1.00 | 40.83 | 6 |
| ATOM | 1200 | CD2 | TRP | A | 292 | 67.757 | 54.662 | 73.878 | 1.00 | 40.51 | 6 |
| ATOM | 1201 | CE2 | TRP | A | 292 | 66.870 | 54.390 | 74.940 | 1.00 | 41.33 | 6 |
| ATOM | 1202 | CE3 | TRP | A | 292 | 69.054 | 55.104 | 74.175 | 1.00 | 42.32 | 6 |
| ATOM | 1203 | CD1 | TRP | A | 292 | 65.803 | 53.987 | 73.019 | 1.00 | 39.80 | 6 |
| ATOM | 1204 | NE1 | TRP | A | 292 | 65.685 | 53.981 | 74.389 | 1.00 | 39.96 | 7 |
| ATOM | 1205 | CZ2 | TRP | A | 292 | 67.238 | 54.544 | 76.284 | 1.00 | 41.81 | 6 |
| ATOM | 1206 | CZ3 | TRP | A | 292 | 69.420 | 55.259 | 75.512 | 1.00 | 41.42 | 6 |
| ATOM | 1207 | CH2 | TRP | A | 292 | 68.512 | 54.978 | 76.549 | 1.00 | 41.81 | 6 |
| ATOM | 1208 | C | TRP | A | 292 | 67.941 | 52.016 | 71.100 | 1.00 | 37.56 | 6 |
| ATOM | 1209 | O | TRP | A | 292 | 67.944 | 51.378 | 72.153 | 1.00 | 38.94 | 8 |
| ATOM | 1210 | N | VAL | A | 293 | 67.336 | 51.591 | 69.996 | 1.00 | 37.67 | 7 |
| ATOM | 1211 | CA | VAL | A | 293 | 66.648 | 50.309 | 69.952 | 1.00 | 38.63 | 6 |
| ATOM | 1212 | CB | VAL | A | 293 | 65.887 | 50.142 | 68.618 | 1.00 | 38.35 | 6 |
| ATOM | 1213 | CG1 | VAL | A | 293 | 65.294 | 48.743 | 68.518 | 1.00 | 38.72 | 6 |
| ATOM | 1214 | CG2 | VAL | A | 293 | 64.783 | 51.187 | 68.523 | 1.00 | 38.53 | 6 |
| ATOM | 1215 | C | VAL | A | 293 | 67.654 | 49.169 | 70.123 | 1.00 | 38.97 | 6 |
| ATOM | 1216 | O | VAL | A | 293 | 67.391 | 48.199 | 70.843 | 1.00 | 38.45 | 8 |
| ATOM | 1217 | N | ARG | A | 294 | 68.808 | 49.290 | 69.471 | 1.00 | 37.88 | 7 |
| ATOM | 1218 | CA | ARG | A | 294 | 69.836 | 48.265 | 69.569 | 1.00 | 38.87 | 6 |
| ATOM | 1219 | CB | ARG | A | 294 | 70.891 | 48.458 | 68.479 | 1.00 | 37.83 | 6 |
| ATOM | 1220 | CG | ARG | A | 294 | 70.336 | 48.304 | 67.073 | 1.00 | 37.55 | 6 |
| ATOM | 1221 | CD | ARG | A | 294 | 71.443 | 48.244 | 66.033 | 1.00 | 38.44 | 6 |
| ATOM | 1222 | NE | ARG | A | 294 | 70.898 | 48.121 | 64.685 | 1.00 | 37.10 | 7 |
| ATOM | 1223 | CZ | ARG | A | 294 | 71.592 | 47.719 | 63.627 | 1.00 | 36.10 | 6 |
| ATOM | 1224 | NH1 | ARG | A | 294 | 72.876 | 47.399 | 63.754 | 1.00 | 33.60 | 7 |
| ATOM | 1225 | NH2 | ARG | A | 294 | 70.992 | 47.616 | 62.447 | 1.00 | 36.18 | 7 |
| ATOM | 1226 | C | ARG | A | 294 | 70.481 | 48.327 | 70.947 | 1.00 | 40.31 | 6 |
| ATOM | 1227 | O | ARG | A | 294 | 70.912 | 47.312 | 71.495 | 1.00 | 38.94 | 8 |
| ATOM | 1228 | N | HIS | A | 295 | 70.537 | 49.532 | 71.504 | 1.00 | 41.82 | 7 |
| ATOM | 1229 | CA | HIS | A | 295 | 71.102 | 49.723 | 72.829 | 1.00 | 43.55 | 6 |
| ATOM | 1230 | CB | HIS | A | 295 | 71.117 | 51.208 | 73.175 | 1.00 | 44.90 | 6 |
| ATOM | 1231 | CG | HIS | A | 295 | 71.411 | 51.485 | 74.613 | 1.00 | 47.15 | 6 |
| ATOM | 1232 | CD2 | HIS | A | 295 | 70.627 | 51.970 | 75.605 | 1.00 | 48.79 | 6 |
| ATOM | 1233 | ND1 | HIS | A | 295 | 72.644 | 51.245 | 75.181 | 1.00 | 47.09 | 7 |
| ATOM | 1234 | CE1 | HIS | A | 295 | 72.607 | 51.571 | 76.461 | 1.00 | 50.25 | 6 |
| ATOM | 1235 | NE2 | HIS | A | 295 | 71.394 | 52.013 | 76.744 | 1.00 | 50.75 | 7 |
| ATOM | 1236 | C | HIS | A | 295 | 70.244 | 48.964 | 73.842 | 1.00 | 45.30 | 6 |
| ATOM | 1237 | O | HIS | A | 295 | 70.765 | 48.230 | 74.684 | 1.00 | 45.38 | 8 |
| ATOM | 1238 | N | CYS | A | 296 | 68.927 | 49.149 | 73.718 | 1.00 | 45.70 | 7 |
| ATOM | 1239 | CA | CYS | A | 296 | 67.980 | 48.485 | 74.610 | 1.00 | 46.87 | 6 |
| ATOM | 1240 | CB | CYS | A | 296 | 66.571 | 49.043 | 74.400 | 1.00 | 45.82 | 6 |
| ATOM | 1241 | SG | CYS | A | 296 | 66.342 | 50.748 | 75.001 | 1.00 | 45.53 | 16 |
| ATOM | 1242 | C | CYS | A | 296 | 67.967 | 46.972 | 74.453 | 1.00 | 48.70 | 6 |
| ATOM | 1243 | O | CYS | A | 296 | 67.840 | 46.266 | 75.460 | 1.00 | 49.54 | 8 |
| ATOM | 1244 | N | LEU | A | 297 | 68.087 | 46.462 | 73.232 | 1.00 | 49.73 | 7 |
| ATOM | 1245 | CA | LEU | A | 297 | 68.093 | 45.017 | 73.052 | 1.00 | 51.79 | 6 |
| ATOM | 1246 | CB | LEU | A | 297 | 68.026 | 44.653 | 71.561 | 1.00 | 50.94 | 6 |
| ATOM | 1247 | CG | LEU | A | 297 | 66.761 | 44.998 | 70.763 | 1.00 | 51.64 | 6 |
| ATOM | 1248 | CD1 | LEU | A | 297 | 66.978 | 44.721 | 69.287 | 1.00 | 51.47 | 6 |
| ATOM | 1249 | CD2 | LEU | A | 297 | 65.549 | 44.235 | 71.296 | 1.00 | 51.62 | 6 |
| ATOM | 1250 | C | LEU | A | 297 | 69.340 | 44.421 | 73.705 | 1.00 | 53.48 | 6 |
| ATOM | 1251 | O | LEU | A | 297 | 69.276 | 43.455 | 74.474 | 1.00 | 53.56 | 8 |
| ATOM | 1252 | N | LYS | A | 298 | 70.495 | 45.016 | 73.394 | 1.00 | 55.12 | 7 |
| ATOM | 1253 | CA | LYS | A | 298 | 71.793 | 44.624 | 73.919 | 1.00 | 57.32 | 6 |
| ATOM | 1254 | CB | LYS | A | 298 | 72.874 | 45.596 | 73.448 | 1.00 | 57.96 | 6 |
| ATOM | 1255 | CG | LYS | A | 298 | 74.270 | 45.263 | 73.948 | 1.00 | 59.97 | 6 |
| ATOM | 1256 | CD | LYS | A | 298 | 75.291 | 46.268 | 73.441 | 1.00 | 60.96 | 6 |
| ATOM | 1257 | CE | LYS | A | 298 | 76.688 | 45.927 | 73.931 | 1.00 | 62.69 | 6 |
| ATOM | 1258 | NZ | LYS | A | 298 | 77.704 | 46.893 | 73.427 | 1.00 | 62.31 | 7 |
| ATOM | 1259 | C | LYS | A | 298 | 71.770 | 44.545 | 75.440 | 1.00 | 58.03 | 6 |
| ATOM | 1260 | O | LYS | A | 298 | 72.358 | 43.651 | 76.045 | 1.00 | 58.09 | 8 |
| ATOM | 1261 | N | ASN | A | 299 | 71.065 | 45.486 | 76.053 | 1.00 | 59.02 | 7 |
| ATOM | 1262 | CA | ASN | A | 299 | 70.964 | 45.516 | 77.505 | 1.00 | 60.84 | 6 |
| ATOM | 1263 | CB | ASN | A | 299 | 71.235 | 46.938 | 78.002 | 1.00 | 60.80 | 6 |
| ATOM | 1264 | CG | ASN | A | 299 | 72.637 | 47.414 | 77.656 | 1.00 | 61.76 | 6 |
| ATOM | 1265 | OD1 | ASN | A | 299 | 73.630 | 46.856 | 78.129 | 1.00 | 62.07 | 8 |
| ATOM | 1266 | ND2 | ASN | A | 299 | 72.725 | 48.443 | 76.822 | 1.00 | 62.96 | 7 |
| ATOM | 1267 | C | ASN | A | 299 | 69.630 | 45.007 | 78.041 | 1.00 | 61.49 | 6 |
| ATOM | 1268 | O | ASN | A | 299 | 69.166 | 45.429 | 79.093 | 1.00 | 62.24 | 8 |
| ATOM | 1269 | N | GLY | A | 300 | 68.994 | 44.091 | 77.310 | 1.00 | 62.34 | 7 |
| ATOM | 1270 | CA | GLY | A | 300 | 67.722 | 43.521 | 77.748 | 1.00 | 62.14 | 6 |
| ATOM | 1271 | C | GLY | A | 300 | 66.754 | 44.525 | 78.376 | 1.00 | 62.98 | 6 |
| ATOM | 1272 | O | GLY | A | 300 | 66.082 | 44.242 | 79.369 | 1.00 | 63.06 | 8 |
| ATOM | 1273 | N | GLU | A | 301 | 66.696 | 45.710 | 77.779 | 1.00 | 63.26 | 7 |
| ATOM | 1274 | CA | GLU | A | 301 | 65.804 | 46.766 | 78.244 | 1.00 | 63.42 | 6 |
| ATOM | 1275 | CB | GLU | A | 301 | 66.504 | 48.114 | 78.189 | 1.00 | 65.10 | 6 |
| ATOM | 1276 | CG | GLU | A | 301 | 67.648 | 48.272 | 79.164 | 1.00 | 68.21 | 6 |
| ATOM | 1277 | CD | GLU | A | 301 | 68.199 | 49.677 | 79.165 | 1.00 | 70.41 | 6 |
| ATOM | 1278 | OE1 | GLU | A | 301 | 69.349 | 49.878 | 79.606 | 1.00 | 71.99 | 8 |
| ATOM | 1279 | OE2 | GLU | A | 301 | 67.463 | 50.578 | 78.735 | 1.00 | 71.25 | 8 |
| ATOM | 1280 | C | GLU | A | 301 | 64.565 | 46.886 | 77.376 | 1.00 | 62.94 | 6 |
| ATOM | 1281 | O | GLU | A | 301 | 64.585 | 46.628 | 76.178 | 1.00 | 63.20 | 8 |
| ATOM | 1282 | N | GLU | A | 302 | 63.506 | 47.287 | 78.043 | 1.00 | 61.90 | 7 |
| ATOM | 1283 | CA | GLU | A | 302 | 62.251 | 47.509 | 77.434 | 1.00 | 61.43 | 6 |
| ATOM | 1284 | CB | GLU | A | 302 | 61.139 | 47.250 | 78.436 | 1.00 | 62.82 | 6 |
| ATOM | 1285 | CG | GLU | A | 302 | 61.434 | 46.050 | 79.331 | 1.00 | 64.81 | 6 |
| ATOM | 1286 | CD | GLU | A | 302 | 60.351 | 45.864 | 80.346 | 1.00 | 66.45 | 6 |
| ATOM | 1287 | OE1 | GLU | A | 302 | 60.040 | 46.831 | 81.076 | 1.00 | 66.95 | 8 |
| ATOM | 1288 | OE2 | GLU | A | 302 | 59.794 | 44.746 | 80.418 | 1.00 | 67.18 | 8 |
| ATOM | 1289 | C | GLU | A | 302 | 62.286 | 48.918 | 76.830 | 1.00 | 59.72 | 6 |
| ATOM | 1290 | O | GLU | A | 302 | 62.725 | 49.880 | 77.466 | 1.00 | 60.18 | 8 |
| ATOM | 1291 | N | ILE | A | 303 | 61.799 | 49.030 | 75.585 | 1.00 | 56.79 | 7 |
| ATOM | 1292 | CA | ILE | A | 303 | 61.780 | 50.286 | 74.798 | 1.00 | 54.83 | 6 |
| ATOM | 1293 | CB | ILE | A | 303 | 61.713 | 49.968 | 73.285 | 1.00 | 54.08 | 6 |
| ATOM | 1294 | CG2 | ILE | A | 303 | 61.716 | 51.259 | 72.476 | 1.00 | 53.73 | 6 |
| ATOM | 1295 | CG1 | ILE | A | 303 | 62.897 | 49.081 | 72.889 | 1.00 | 53.15 | 6 |
| ATOM | 1296 | CD1 | ILE | A | 303 | 62.862 | 48.615 | 71.449 | 1.00 | 51.54 | 6 |
| ATOM | 1297 | C | ILE | A | 303 | 60.629 | 51.219 | 75.164 | 1.00 | 53.89 | 6 |
| ATOM | 1298 | O | ILE | A | 303 | 59.505 | 51.042 | 74.694 | 1.00 | 54.29 | 8 |
| ATOM | 1299 | N | HIS | A | 304 | 60.913 | 52.211 | 76.002 | 1.00 | 52.88 | 7 |
| ATOM | 1300 | CA | HIS | A | 304 | 59.898 | 53.172 | 76.422 | 1.00 | 51.29 | 6 |
| ATOM | 1301 | CB | HIS | A | 304 | 60.037 | 53.503 | 77.911 | 1.00 | 52.76 | 6 |
| ATOM | 1302 | CG | HIS | A | 304 | 59.715 | 52.359 | 78.818 | 1.00 | 54.08 | 6 |
| ATOM | 1303 | CD2 | HIS | A | 304 | 58.615 | 52.094 | 79.562 | 1.00 | 54.23 | 6 |
| ATOM | 1304 | ND1 | HIS | A | 304 | 60.577 | 51.303 | 79.022 | 1.00 | 55.17 | 7 |
| ATOM | 1305 | CE1 | HIS | A | 304 | 60.023 | 50.437 | 79.853 | 1.00 | 55.51 | 6 |
| ATOM | 1306 | NE2 | HIS | A | 304 | 58.832 | 50.895 | 80.195 | 1.00 | 54.22 | 7 |
| ATOM | 1307 | C | HIS | A | 304 | 59.960 | 54.464 | 75.623 | 1.00 | 50.52 | 6 |
| ATOM | 1308 | O | HIS | A | 304 | 61.018 | 55.086 | 75.490 | 1.00 | 48.43 | 8 |
| ATOM | 1309 | N | VAL | A | 305 | 58.809 | 54.868 | 75.105 | 1.00 | 49.76 | 7 |
| ATOM | 1310 | CA | VAL | A | 305 | 58.718 | 56.079 | 74.317 | 1.00 | 49.67 | 6 |
| ATOM | 1311 | CB | VAL | A | 305 | 58.760 | 55.749 | 72.810 | 1.00 | 50.96 | 6 |
| ATOM | 1312 | CG1 | VAL | A | 305 | 58.515 | 57.002 | 71.989 | 1.00 | 51.45 | 6 |
| ATOM | 1313 | CG2 | VAL | A | 305 | 60.100 | 55.116 | 72.430 | 1.00 | 50.42 | 6 |
| ATOM | 1314 | C | VAL | A | 305 | 57.472 | 56.846 | 74.628 | 1.00 | 49.29 | 6 |
| ATOM | 1315 | O | VAL | A | 305 | 56.403 | 56.272 | 74.903 | 1.00 | 47.84 | 8 |
| ATOM | 1316 | N | VAL | A | 306 | 57.680 | 58.124 | 74.641 | 1.00 | 50.50 | 7 |
| ATOM | 1317 | CA | VAL | A | 306 | 56.583 | 58.986 | 74.822 | 1.00 | 51.75 | 6 |
| ATOM | 1318 | CB | VAL | A | 306 | 56.845 | 60.116 | 75.822 | 1.00 | 51.55 | 6 |
| ATOM | 1319 | CG1 | VAL | A | 306 | 58.048 | 60.941 | 75.407 | 1.00 | 51.41 | 6 |
| ATOM | 1320 | CG2 | VAL | A | 306 | 55.618 | 60.995 | 75.969 | 1.00 | 51.51 | 6 |
| ATOM | 1321 | C | VAL | A | 306 | 56.317 | 59.521 | 73.489 | 1.00 | 53.05 | 6 |
| ATOM | 1322 | O | VAL | A | 306 | 57.150 | 59.543 | 72.588 | 1.00 | 53.60 | 8 |
| ATOM | 1323 | N | LEU | A | 307 | 55.089 | 59.936 | 73.386 | 1.00 | 54.30 | 7 |
| ATOM | 1324 | CA | LEU | A | 307 | 54.641 | 60.453 | 72.150 | 1.00 | 54.99 | 6 |
| ATOM | 1325 | CB | LEU | A | 307 | 53.542 | 59.555 | 71.616 | 1.00 | 55.78 | 6 |
| ATOM | 1326 | CG | LEU | A | 307 | 52.238 | 60.189 | 71.225 | 1.00 | 56.64 | 6 |
| ATOM | 1327 | CD1 | LEU | A | 307 | 52.363 | 60.868 | 69.868 | 1.00 | 57.75 | 6 |
| ATOM | 1328 | CD2 | LEU | A | 307 | 51.125 | 59.161 | 71.200 | 1.00 | 57.94 | 6 |
| ATOM | 1329 | C | LEU | A | 307 | 54.257 | 61.869 | 72.358 | 1.00 | 55.54 | 6 |
| ATOM | 1330 | O | LEU | A | 307 | 53.246 | 62.177 | 72.990 | 1.00 | 55.97 | 8 |
| ATOM | 1331 | N | ASP | A | 308 | 55.063 | 62.716 | 71.857 | 1.00 | 56.07 | 7 |
| ATOM | 1332 | CA | ASP | A | 308 | 54.682 | 64.064 | 72.043 | 1.00 | 56.52 | 6 |
| ATOM | 1333 | CB | ASP | A | 308 | 55.602 | 64.748 | 73.045 | 1.00 | 58.12 | 6 |
| ATOM | 1334 | CG | ASP | A | 308 | 56.883 | 65.264 | 72.398 | 1.00 | 59.98 | 6 |
| ATOM | 1335 | OD1 | ASP | A | 308 | 57.109 | 64.963 | 71.212 | 1.00 | 60.97 | 8 |
| ATOM | 1336 | OD2 | ASP | A | 308 | 57.646 | 65.967 | 73.086 | 1.00 | 61.57 | 8 |
| ATOM | 1337 | C | ASP | A | 308 | 54.632 | 64.802 | 70.756 | 1.00 | 55.87 | 6 |
| ATOM | 1338 | O | ASP | A | 308 | 54.464 | 64.261 | 69.678 | 1.00 | 54.53 | 8 |
| ATOM | 1339 | N | THR | A | 309 | 54.791 | 66.053 | 70.940 | 1.00 | 55.96 | 7 |
| ATOM | 1340 | CA | THR | A | 309 | 54.712 | 66.969 | 69.877 | 1.00 | 57.44 | 6 |
| ATOM | 1341 | CB | THR | A | 309 | 53.688 | 68.047 | 70.261 | 1.00 | 58.70 | 6 |
| ATOM | 1342 | OG1 | THR | A | 309 | 53.666 | 69.060 | 69.262 | 1.00 | 62.01 | 8 |
| ATOM | 1343 | CG2 | THR | A | 309 | 54.038 | 68.650 | 71.600 | 1.00 | 59.88 | 6 |
| ATOM | 1344 | C | THR | A | 309 | 56.069 | 67.500 | 69.560 | 1.00 | 56.32 | 6 |
| ATOM | 1345 | O | THR | A | 309 | 56.795 | 68.001 | 70.416 | 1.00 | 56.02 | 8 |
| ATOM | 1346 | N | PRO | A | 310 | 56.378 | 67.396 | 68.283 | 1.00 | 55.23 | 7 |
| ATOM | 1347 | CD | PRO | A | 310 | 55.980 | 66.187 | 67.528 | 1.00 | 54.52 | 6 |
| ATOM | 1348 | CA | PRO | A | 310 | 57.695 | 67.827 | 67.794 | 1.00 | 53.52 | 6 |
| ATOM | 1349 | CB | PRO | A | 310 | 57.591 | 67.539 | 66.321 | 1.00 | 53.15 | 6 |
| ATOM | 1350 | CG | PRO | A | 310 | 56.819 | 66.260 | 66.275 | 1.00 | 54.43 | 6 |
| ATOM | 1351 | C | PRO | A | 310 | 58.313 | 69.167 | 68.107 | 1.00 | 51.85 | 6 |
| ATOM | 1352 | O | PRO | A | 310 | 57.680 | 70.126 | 68.527 | 1.00 | 52.18 | 8 |
| ATOM | 1353 | N | PRO | A | 311 | 59.619 | 69.160 | 67.855 | 1.00 | 49.76 | 7 |
| ATOM | 1354 | CD | PRO | A | 311 | 60.454 | 68.196 | 68.582 | 1.00 | 48.77 | 6 |
| ATOM | 1355 | CA | PRO | A | 311 | 60.323 | 70.419 | 67.909 | 1.00 | 47.64 | 6 |
| ATOM | 1356 | CB | PRO | A | 311 | 61.764 | 70.035 | 67.723 | 1.00 | 46.84 | 6 |
| ATOM | 1357 | CG | PRO | A | 311 | 61.843 | 68.768 | 68.487 | 1.00 | 47.27 | 6 |
| ATOM | 1358 | C | PRO | A | 311 | 59.737 | 71.345 | 66.845 | 1.00 | 46.76 | 6 |
| ATOM | 1359 | O | PRO | A | 311 | 58.810 | 70.970 | 66.136 | 1.00 | 47.65 | 8 |
| ATOM | 1360 | N | ASP | A | 312 | 60.300 | 72.540 | 66.741 | 1.00 | 45.62 | 7 |
| ATOM | 1361 | CA | ASP | A | 312 | 59.994 | 73.491 | 65.675 | 1.00 | 45.14 | 6 |
| ATOM | 1362 | CB | ASP | A | 312 | 59.123 | 74.725 | 66.077 | 1.00 | 46.67 | 6 |
| ATOM | 1363 | CG | ASP | A | 312 | 58.272 | 75.363 | 64.945 | 1.00 | 48.73 | 6 |
| ATOM | 1364 | OD1 | ASP | A | 312 | 57.944 | 74.661 | 63.978 | 1.00 | 51.04 | 8 |
| ATOM | 1365 | OD2 | ASP | A | 312 | 57.954 | 76.568 | 65.049 | 1.00 | 50.35 | 8 |
| ATOM | 1366 | C | ASP | A | 312 | 61.354 | 73.897 | 65.130 | 1.00 | 43.48 | 6 |
| ATOM | 1367 | O | ASP | A | 312 | 62.254 | 74.345 | 65.832 | 1.00 | 41.15 | 8 |
| ATOM | 1368 | N | PRO | A | 313 | 61.431 | 73.684 | 63.821 | 1.00 | 43.57 | 7 |
| ATOM | 1369 | CD | PRO | A | 313 | 60.894 | 72.424 | 63.257 | 1.00 | 44.66 | 6 |
| ATOM | 1370 | CA | PRO | A | 313 | 62.636 | 74.035 | 63.099 | 1.00 | 42.37 | 6 |
| ATOM | 1371 | CB | PRO | A | 313 | 62.296 | 73.637 | 61.660 | 1.00 | 44.00 | 6 |
| ATOM | 1372 | CG | PRO | A | 313 | 61.504 | 72.369 | 61.889 | 1.00 | 45.07 | 6 |
| ATOM | 1373 | C | PRO | A | 313 | 63.069 | 75.443 | 63.373 | 1.00 | 41.67 | 6 |
| ATOM | 1374 | O | PRO | A | 313 | 64.245 | 75.780 | 63.557 | 1.00 | 40.31 | 8 |
| ATOM | 1375 | N | ALA | A | 314 | 61.993 | 76.241 | 63.376 | 1.00 | 40.40 | 7 |
| ATOM | 1376 | CA | ALA | A | 314 | 62.038 | 77.684 | 63.573 | 1.00 | 41.14 | 6 |
| ATOM | 1377 | CB | ALA | A | 314 | 60.673 | 78.235 | 63.965 | 1.00 | 42.55 | 6 |
| ATOM | 1378 | C | ALA | A | 314 | 63.062 | 78.016 | 64.614 | 1.00 | 40.28 | 6 |
| ATOM | 1379 | O | ALA | A | 314 | 63.717 | 79.066 | 64.558 | 1.00 | 40.81 | 8 |
| ATOM | 1380 | N | LEU | A | 315 | 63.177 | 77.141 | 65.548 | 1.00 | 39.76 | 7 |
| ATOM | 1381 | CA | LEU | A | 315 | 64.094 | 77.421 | 66.601 | 1.00 | 39.55 | 6 |
| ATOM | 1382 | CB | LEU | A | 315 | 63.711 | 76.577 | 67.813 | 1.00 | 39.74 | 6 |
| ATOM | 1383 | CG | LEU | A | 315 | 62.193 | 76.479 | 68.084 | 1.00 | 40.73 | 6 |
| ATOM | 1384 | CD1 | LEU | A | 315 | 61.924 | 75.629 | 69.314 | 1.00 | 41.09 | 6 |
| ATOM | 1385 | CD2 | LEU | A | 315 | 61.579 | 77.862 | 68.250 | 1.00 | 40.50 | 6 |
| ATOM | 1386 | C | LEU | A | 315 | 65.539 | 77.292 | 66.181 | 1.00 | 39.90 | 6 |
| ATOM | 1387 | O | LEU | A | 315 | 66.426 | 77.920 | 66.773 | 1.00 | 38.56 | 8 |
| ATOM | 1388 | N | ASP | A | 316 | 65.805 | 76.488 | 65.158 | 1.00 | 39.78 | 7 |
| ATOM | 1389 | CA | ASP | A | 316 | 67.155 | 76.290 | 64.729 | 1.00 | 40.86 | 6 |
| ATOM | 1390 | CB | ASP | A | 316 | 67.293 | 74.918 | 64.082 | 1.00 | 40.22 | 6 |
| ATOM | 1391 | CG | ASP | A | 316 | 67.031 | 73.773 | 65.044 | 1.00 | 40.97 | 6 |
| ATOM | 1392 | OD1 | ASP | A | 316 | 67.547 | 73.832 | 66.184 | 1.00 | 40.43 | 8 |
| ATOM | 1393 | OD2 | ASP | A | 316 | 66.324 | 72.813 | 64.666 | 1.00 | 38.10 | 8 |
| ATOM | 1394 | C | ASP | A | 316 | 67.630 | 77.379 | 63.790 | 1.00 | 40.87 | 6 |
| ATOM | 1395 | O | ASP | A | 316 | 68.686 | 77.268 | 63.171 | 1.00 | 40.07 | 8 |
| ATOM | 1396 | N | GLU | A | 317 | 66.861 | 78.466 | 63.699 | 1.00 | 43.50 | 7 |
| ATOM | 1397 | CA | GLU | A | 317 | 67.114 | 79.574 | 62.764 | 1.00 | 45.32 | 6 |
| ATOM | 1398 | CB | GLU | A | 317 | 66.135 | 80.700 | 63.077 | 1.00 | 48.53 | 6 |
| ATOM | 1399 | CG | GLU | A | 317 | 64.863 | 80.574 | 62.271 | 1.00 | 53.85 | 6 |
| ATOM | 1400 | CD | GLU | A | 317 | 63.842 | 81.630 | 62.609 | 1.00 | 55.53 | 6 |
| ATOM | 1401 | OE1 | GLU | A | 317 | 64.221 | 82.817 | 62.677 | 1.00 | 56.76 | 8 |
| ATOM | 1402 | OE2 | GLU | A | 317 | 62.662 | 81.269 | 62.820 | 1.00 | 57.08 | 8 |
| ATOM | 1403 | C | GLU | A | 317 | 68.568 | 80.069 | 62.657 | 1.00 | 45.87 | 6 |
| ATOM | 1404 | O | GLU | A | 317 | 69.258 | 80.246 | 63.674 | 1.00 | 44.16 | 8 |
| ATOM | 1405 | N | VAL | A | 318 | 68.999 | 80.265 | 61.425 | 1.00 | 45.46 | 7 |
| ATOM | 1406 | CA | VAL | A | 318 | 70.330 | 80.776 | 61.230 | 1.00 | 46.54 | 6 |
| ATOM | 1407 | CB | VAL | A | 318 | 70.952 | 80.279 | 59.906 | 1.00 | 45.15 | 6 |
| ATOM | 1408 | CG1 | VAL | A | 318 | 72.182 | 81.100 | 59.557 | 1.00 | 42.41 | 6 |
| ATOM | 1409 | CG2 | VAL | A | 318 | 71.285 | 78.800 | 60.002 | 1.00 | 42.47 | 6 |
| ATOM | 1410 | C | VAL | A | 318 | 70.199 | 82.260 | 61.218 | 1.00 | 49.57 | 6 |
| ATOM | 1411 | O | VAL | A | 318 | 69.080 | 82.786 | 61.204 | 1.00 | 49.13 | 8 |
| ATOM | 1412 | N | ARG | A | 319 | 71.354 | 82.981 | 61.240 | 1.00 | 52.58 | 7 |
| ATOM | 1413 | CA | ARG | A | 319 | 71.411 | 84.444 | 61.150 | 1.00 | 55.97 | 6 |
| ATOM | 1414 | CB | ARG | A | 319 | 72.163 | 85.077 | 62.340 | 1.00 | 57.05 | 6 |
| ATOM | 1415 | CG | ARG | A | 319 | 71.952 | 86.580 | 62.518 | 1.00 | 58.76 | 6 |
| ATOM | 1416 | CD | ARG | A | 319 | 71.761 | 86.996 | 63.966 | 1.00 | 60.55 | 6 |
| ATOM | 1417 | NE | ARG | A | 319 | 71.877 | 88.434 | 64.157 | 1.00 | 62.82 | 7 |
| ATOM | 1418 | CZ | ARG | A | 319 | 72.333 | 89.004 | 65.256 | 1.00 | 64.04 | 6 |
| ATOM | 1419 | NH1 | ARG | A | 319 | 72.732 | 88.253 | 66.283 | 1.00 | 64.96 | 7 |
| ATOM | 1420 | NH2 | ARG | A | 319 | 72.390 | 90.323 | 65.326 | 1.00 | 64.36 | 7 |
| ATOM | 1421 | C | ARG | A | 319 | 72.029 | 84.853 | 59.801 | 1.00 | 58.67 | 6 |
| ATOM | 1422 | O | ARG | A | 319 | 72.970 | 84.249 | 59.283 | 1.00 | 58.69 | 8 |
| ATOM | 1423 | N | LYS | A | 320 | 71.412 | 85.914 | 59.252 | 1.00 | 61.11 | 7 |
| ATOM | 1424 | CA | LYS | A | 320 | 71.675 | 86.527 | 57.933 | 1.00 | 63.48 | 6 |
| ATOM | 1425 | CB | LYS | A | 320 | 70.771 | 87.770 | 57.854 | 1.00 | 63.99 | 6 |
| ATOM | 1426 | CG | LYS | A | 320 | 69.631 | 87.801 | 58.886 | 1.00 | 65.14 | 6 |
| ATOM | 1427 | CD | LYS | A | 320 | 68.303 | 87.312 | 58.330 | 1.00 | 65.59 | 6 |
| ATOM | 1428 | CE | LYS | A | 320 | 67.348 | 86.893 | 59.451 | 1.00 | 66.14 | 6 |
| ATOM | 1429 | NZ | LYS | A | 320 | 66.382 | 87.976 | 59.787 | 1.00 | 64.88 | 7 |
| ATOM | 1430 | C | LYS | A | 320 | 73.160 | 86.782 | 57.653 | 1.00 | 64.80 | 6 |
| ATOM | 1431 | O | LYS | A | 320 | 73.949 | 85.840 | 57.816 | 1.00 | 64.57 | 8 |
| ATOM | 1432 | N | GLU | A | 321 | 73.592 | 87.975 | 57.236 | 1.00 | 67.34 | 7 |
| ATOM | 1433 | CA | GLU | A | 321 | 75.021 | 88.148 | 56.989 | 1.00 | 69.48 | 6 |
| ATOM | 1434 | CB | GLU | A | 321 | 75.291 | 88.804 | 55.607 | 1.00 | 70.55 | 6 |
| ATOM | 1435 | CG | GLU | A | 321 | 76.510 | 88.309 | 54.810 | 1.00 | 73.06 | 6 |
| ATOM | 1436 | CD | GLU | A | 321 | 76.926 | 89.208 | 53.625 | 1.00 | 73.59 | 6 |
| ATOM | 1437 | OE1 | GLU | A | 321 | 77.733 | 90.136 | 53.841 | 1.00 | 74.86 | 8 |
| ATOM | 1438 | OE2 | GLU | A | 321 | 76.426 | 88.982 | 52.504 | 1.00 | 73.72 | 8 |
| ATOM | 1439 | C | GLU | A | 321 | 75.612 | 89.000 | 58.104 | 1.00 | 70.05 | 6 |
| ATOM | 1440 | O | GLU | A | 321 | 75.030 | 89.989 | 58.547 | 1.00 | 70.61 | 8 |
| ATOM | 1441 | N | GLY | A | 322 | 76.802 | 88.604 | 58.568 | 1.00 | 70.18 | 7 |
| ATOM | 1442 | CA | GLY | A | 322 | 77.464 | 89.337 | 59.616 | 1.00 | 70.44 | 6 |
| ATOM | 1443 | C | GLY | A | 322 | 78.955 | 89.516 | 59.308 | 1.00 | 69.97 | 6 |
| ATOM | 1444 | O | GLY | A | 322 | 79.350 | 90.440 | 58.592 | 1.00 | 69.63 | 8 |
| ATOM | 1445 | N | VAL | A | 352 | 103.335 | 84.397 | 36.291 | 1.00 | 26.24 | 7 |
| ATOM | 1446 | CA | VAL | A | 352 | 103.442 | 85.790 | 36.752 | 1.00 | 26.24 | 6 |
| ATOM | 1447 | CB | VAL | A | 352 | 102.751 | 86.747 | 35.740 | 1.00 | 26.24 | 6 |
| ATOM | 1448 | CG1 | VAL | A | 352 | 101.328 | 87.062 | 36.186 | 1.00 | 26.24 | 6 |
| ATOM | 1449 | CG2 | VAL | A | 352 | 103.556 | 88.031 | 35.568 | 1.00 | 26.24 | 6 |
| ATOM | 1450 | C | VAL | A | 352 | 102.862 | 85.928 | 38.164 | 1.00 | 26.24 | 6 |
| ATOM | 1451 | O | VAL | A | 352 | 101.646 | 85.967 | 38.372 | 1.00 | 26.24 | 8 |
| ATOM | 1452 | N | SER | A | 353 | 103.818 | 86.014 | 39.126 | 1.00 | 26.24 | 7 |
| ATOM | 1453 | CA | SER | A | 353 | 103.604 | 86.226 | 40.578 | 1.00 | 26.24 | 6 |
| ATOM | 1454 | CB | SER | A | 353 | 103.572 | 84.921 | 41.381 | 1.00 | 26.24 | 6 |
| ATOM | 1455 | OG | SER | A | 353 | 103.350 | 85.181 | 42.757 | 1.00 | 26.24 | 8 |
| ATOM | 1456 | C | SER | A | 353 | 104.740 | 87.100 | 41.071 | 1.00 | 26.24 | 6 |
| ATOM | 1457 | O | SER | A | 353 | 104.486 | 88.186 | 41.577 | 1.00 | 26.24 | 8 |
| ATOM | 1458 | N | LEU | A | 354 | 106.046 | 86.657 | 40.926 | 1.00 | 103.21 | 7 |
| ATOM | 1459 | CA | LEU | A | 354 | 107.143 | 87.565 | 41.352 | 1.00 | 103.21 | 6 |
| ATOM | 1460 | CB | LEU | A | 354 | 107.153 | 87.678 | 42.858 | 1.00 | 65.64 | 6 |
| ATOM | 1461 | CG | LEU | A | 354 | 106.487 | 88.947 | 43.392 | 1.00 | 26.24 | 6 |
| ATOM | 1462 | CD1 | LEU | A | 354 | 106.818 | 90.135 | 42.493 | 1.00 | 26.24 | 6 |
| ATOM | 1463 | CD2 | LEU | A | 354 | 104.982 | 88.775 | 43.508 | 1.00 | 26.24 | 6 |
| ATOM | 1464 | C | LEU | A | 354 | 108.570 | 87.324 | 40.796 | 1.00 | 103.21 | 6 |
| ATOM | 1465 | O | LEU | A | 354 | 108.800 | 86.446 | 39.979 | 1.00 | 103.21 | 8 |
| ATOM | 1466 | N | TRP | A | 355 | 109.518 | 88.186 | 41.296 | 1.00 | 63.62 | 7 |
| ATOM | 1467 | CA | TRP | A | 355 | 110.939 | 88.231 | 40.899 | 1.00 | 63.62 | 6 |
| ATOM | 1468 | CB | TRP | A | 355 | 111.746 | 89.071 | 41.965 | 1.00 | 95.68 | 6 |
| ATOM | 1469 | CG | TRP | A | 355 | 112.853 | 90.012 | 41.430 | 1.00 | 26.24 | 6 |
| ATOM | 1470 | CD2 | TRP | A | 355 | 113.385 | 91.245 | 41.996 | 1.00 | 26.24 | 6 |
| ATOM | 1471 | CE2 | TRP | A | 355 | 114.462 | 91.655 | 41.183 | 1.00 | 26.24 | 6 |
| ATOM | 1472 | CE3 | TRP | A | 355 | 113.056 | 92.011 | 43.116 | 1.00 | 26.24 | 6 |
| ATOM | 1473 | CD1 | TRP | A | 355 | 113.568 | 89.818 | 40.284 | 1.00 | 26.24 | 6 |
| ATOM | 1474 | NE1 | TRP | A | 355 | 114.533 | 90.789 | 40.122 | 1.00 | 26.24 | 7 |
| ATOM | 1475 | CZ2 | TRP | A | 355 | 115.277 | 92.735 | 41.517 | 1.00 | 26.24 | 6 |
| ATOM | 1476 | CZ3 | TRP | A | 355 | 113.834 | 93.114 | 43.414 | 1.00 | 26.24 | 6 |
| ATOM | 1477 | CH2 | TRP | A | 355 | 114.931 | 93.465 | 42.619 | 1.00 | 26.24 | 6 |
| ATOM | 1478 | C | TRP | A | 355 | 111.378 | 86.785 | 40.595 | 1.00 | 63.62 | 6 |
| ATOM | 1479 | O | TRP | A | 355 | 110.563 | 85.867 | 40.683 | 1.00 | 63.62 | 8 |
| ATOM | 1480 | N | ASP | A | 356 | 112.637 | 86.598 | 40.233 | 1.00 | 65.79 | 7 |
| ATOM | 1481 | CA | ASP | A | 356 | 113.195 | 85.304 | 39.834 | 1.00 | 65.79 | 6 |
| ATOM | 1482 | CB | ASP | A | 356 | 114.713 | 85.375 | 39.816 | 1.00 | 53.16 | 6 |
| ATOM | 1483 | CG | ASP | A | 356 | 115.243 | 85.665 | 38.423 | 1.00 | 26.24 | 6 |
| ATOM | 1484 | OD1 | ASP | A | 356 | 114.524 | 86.306 | 37.632 | 1.00 | 26.24 | 8 |
| ATOM | 1485 | OD2 | ASP | A | 356 | 116.384 | 85.261 | 38.134 | 1.00 | 26.24 | 8 |
| ATOM | 1486 | C | ASP | A | 356 | 112.715 | 84.103 | 40.653 | 1.00 | 65.79 | 6 |
| ATOM | 1487 | O | ASP | A | 356 | 112.717 | 84.078 | 41.883 | 1.00 | 65.79 | 8 |
| ATOM | 1488 | N | CYS | A | 357 | 112.316 | 83.118 | 39.876 | 1.00 | 92.44 | 7 |
| ATOM | 1489 | CA | CYS | A | 357 | 111.897 | 81.852 | 40.399 | 1.00 | 91.69 | 6 |
| ATOM | 1490 | CB | CYS | A | 357 | 110.882 | 81.158 | 39.481 | 1.00 | 110.85 | 6 |
| ATOM | 1491 | SG | CYS | A | 357 | 110.491 | 82.091 | 37.960 | 1.00 | 115.10 | 16 |
| ATOM | 1492 | C | CYS | A | 357 | 113.107 | 80.990 | 40.577 | 1.00 | 89.52 | 6 |
| ATOM | 1493 | O | CYS | A | 357 | 113.078 | 80.005 | 41.336 | 1.00 | 89.38 | 8 |
| ATOM | 1494 | N | ASP | A | 358 | 114.175 | 81.339 | 39.880 | 1.00 | 56.98 | 7 |
| ATOM | 1495 | CA | ASP | A | 358 | 115.329 | 80.501 | 39.998 | 1.00 | 56.17 | 6 |
| ATOM | 1496 | CB | ASP | A | 358 | 115.914 | 80.267 | 38.614 | 1.00 | 57.77 | 6 |
| ATOM | 1497 | CG | ASP | A | 358 | 114.860 | 79.603 | 37.761 | 1.00 | 58.50 | 6 |
| ATOM | 1498 | OD1 | ASP | A | 358 | 113.921 | 80.315 | 37.351 | 1.00 | 58.37 | 8 |
| ATOM | 1499 | OD2 | ASP | A | 358 | 114.965 | 78.381 | 37.498 | 1.00 | 59.34 | 8 |
| ATOM | 1500 | C | ASP | A | 358 | 116.355 | 80.998 | 40.994 | 1.00 | 55.12 | 6 |
| ATOM | 1501 | O | ASP | A | 358 | 117.469 | 80.497 | 41.014 | 1.00 | 55.96 | 8 |
| ATOM | 1502 | N | ARG | A | 359 | 116.010 | 81.983 | 41.819 | 1.00 | 52.67 | 7 |
| ATOM | 1503 | CA | ARG | A | 359 | 116.956 | 82.480 | 42.833 | 1.00 | 50.51 | 6 |
| ATOM | 1504 | CB | ARG | A | 359 | 116.553 | 83.870 | 43.293 | 1.00 | 53.35 | 6 |
| ATOM | 1505 | CG | ARG | A | 359 | 116.439 | 84.924 | 42.201 | 1.00 | 58.31 | 6 |
| ATOM | 1506 | CD | ARG | A | 359 | 116.130 | 86.308 | 42.786 | 1.00 | 61.85 | 6 |
| ATOM | 1507 | NE | ARG | A | 359 | 116.144 | 87.347 | 41.763 | 1.00 | 65.28 | 7 |
| ATOM | 1508 | CZ | ARG | A | 359 | 115.973 | 88.639 | 42.014 | 1.00 | 66.54 | 6 |
| ATOM | 1509 | NH1 | ARG | A | 359 | 115.774 | 89.051 | 43.259 | 1.00 | 67.51 | 7 |
| ATOM | 1510 | NH2 | ARG | A | 359 | 116.002 | 89.518 | 41.022 | 1.00 | 66.69 | 7 |
| ATOM | 1511 | C | ARG | A | 359 | 117.021 | 81.539 | 44.060 | 1.00 | 46.31 | 6 |
| ATOM | 1512 | O | ARG | A | 359 | 115.983 | 81.023 | 44.486 | 1.00 | 44.79 | 8 |
| ATOM | 1513 | N | LYS | A | 360 | 118.200 | 81.309 | 44.609 | 1.00 | 43.23 | 7 |
| ATOM | 1514 | CA | LYS | A | 360 | 118.175 | 80.438 | 45.764 | 1.00 | 41.85 | 6 |
| ATOM | 1515 | CB | LYS | A | 360 | 119.578 | 79.931 | 46.094 | 1.00 | 41.65 | 6 |
| ATOM | 1516 | CG | LYS | A | 360 | 120.087 | 78.851 | 45.166 | 1.00 | 44.19 | 6 |
| ATOM | 1517 | CD | LYS | A | 360 | 121.244 | 78.095 | 45.801 | 1.00 | 46.49 | 6 |
| ATOM | 1518 | CE | LYS | A | 360 | 121.715 | 76.970 | 44.903 | 1.00 | 49.88 | 6 |
| ATOM | 1519 | NZ | LYS | A | 360 | 122.727 | 76.116 | 45.581 | 1.00 | 48.76 | 7 |
| ATOM | 1520 | C | LYS | A | 360 | 117.549 | 81.192 | 46.931 | 1.00 | 40.20 | 6 |
| ATOM | 1521 | O | LYS | A | 360 | 117.792 | 82.397 | 47.109 | 1.00 | 38.60 | 8 |
| ATOM | 1522 | N | PHE | A | 361 | 116.740 | 80.490 | 47.694 | 1.00 | 37.87 | 7 |
| ATOM | 1523 | CA | PHE | A | 361 | 116.031 | 81.087 | 48.836 | 1.00 | 36.51 | 6 |
| ATOM | 1524 | CB | PHE | A | 361 | 115.009 | 80.087 | 49.396 | 1.00 | 35.26 | 6 |
| ATOM | 1525 | CG | PHE | A | 361 | 114.312 | 80.560 | 50.648 | 1.00 | 32.01 | 6 |
| ATOM | 1526 | CD1 | PHE | A | 361 | 113.179 | 81.363 | 50.574 | 1.00 | 33.24 | 6 |
| ATOM | 1527 | CD2 | PHE | A | 361 | 114.796 | 80.198 | 51.905 | 1.00 | 33.24 | 6 |
| ATOM | 1528 | CE1 | PHE | A | 361 | 112.529 | 81.800 | 51.737 | 1.00 | 28.76 | 6 |
| ATOM | 1529 | CE2 | PHE | A | 361 | 114.155 | 80.629 | 53.073 | 1.00 | 31.87 | 6 |
| ATOM | 1530 | CZ | PHE | A | 361 | 113.018 | 81.432 | 52.988 | 1.00 | 27.51 | 6 |
| ATOM | 1531 | C | PHE | A | 361 | 116.997 | 81.482 | 49.939 | 1.00 | 35.70 | 6 |
| ATOM | 1532 | O | PHE | A | 361 | 117.973 | 80.785 | 50.206 | 1.00 | 36.25 | 8 |
| ATOM | 1533 | N | ARG | A | 362 | 116.730 | 82.618 | 50.566 | 1.00 | 34.72 | 7 |
| ATOM | 1534 | CA | ARG | A | 362 | 117.564 | 83.108 | 51.659 | 1.00 | 33.83 | 6 |
| ATOM | 1535 | CB | ARG | A | 362 | 118.721 | 83.948 | 51.121 | 1.00 | 34.92 | 6 |
| ATOM | 1536 | CG | ARG | A | 362 | 118.292 | 85.259 | 50.487 | 1.00 | 38.15 | 6 |
| ATOM | 1537 | CD | ARG | A | 362 | 119.496 | 86.139 | 50.209 | 1.00 | 40.29 | 6 |
| ATOM | 1538 | NE | ARG | A | 362 | 119.076 | 87.443 | 49.705 | 1.00 | 40.41 | 7 |
| ATOM | 1539 | CZ | ARG | A | 362 | 119.900 | 88.461 | 49.468 | 1.00 | 41.94 | 6 |
| ATOM | 1540 | NH1 | ARG | A | 362 | 121.204 | 88.336 | 49.687 | 1.00 | 39.16 | 7 |
| ATOM | 1541 | NH2 | ARG | A | 362 | 119.415 | 89.609 | 49.016 | 1.00 | 39.73 | 7 |
| ATOM | 1542 | C | ARG | A | 362 | 116.708 | 83.860 | 52.641 | 1.00 | 31.15 | 6 |
| ATOM | 1543 | O | ARG | A | 362 | 115.603 | 84.297 | 52.333 | 1.00 | 29.33 | 8 |
| ATOM | 1544 | N | VAL | A | 363 | 117.226 | 83.999 | 53.816 | 1.00 | 30.99 | 7 |
| ATOM | 1545 | CA | VAL | A | 363 | 116.528 | 84.616 | 54.908 | 1.00 | 31.35 | 6 |
| ATOM | 1546 | CB | VAL | A | 363 | 115.815 | 83.535 | 55.758 | 1.00 | 30.32 | 6 |
| ATOM | 1547 | CG1 | VAL | A | 363 | 116.811 | 82.521 | 56.305 | 1.00 | 33.75 | 6 |
| ATOM | 1548 | CG2 | VAL | A | 363 | 115.038 | 84.176 | 56.888 | 1.00 | 30.23 | 6 |
| ATOM | 1549 | C | VAL | A | 363 | 117.523 | 85.292 | 55.763 | 1.00 | 30.54 | 6 |
| ATOM | 1550 | O | VAL | A | 363 | 118.559 | 84.701 | 56.066 | 1.00 | 30.53 | 8 |
| ATOM | 1551 | N | LYS | A | 364 | 117.279 | 86.523 | 56.179 | 1.00 | 29.22 | 7 |
| ATOM | 1552 | CA | LYS | A | 364 | 118.227 | 87.171 | 57.094 | 1.00 | 29.03 | 6 |
| ATOM | 1553 | CB | LYS | A | 364 | 118.440 | 88.651 | 56.751 | 1.00 | 30.92 | 6 |
| ATOM | 1554 | CG | LYS | A | 364 | 119.319 | 89.358 | 57.751 | 1.00 | 31.02 | 6 |
| ATOM | 1555 | CD | LYS | A | 364 | 119.440 | 90.844 | 57.427 | 1.00 | 34.24 | 6 |
| ATOM | 1556 | CE | LYS | A | 364 | 120.285 | 91.075 | 56.179 | 1.00 | 37.73 | 6 |
| ATOM | 1557 | NZ | LYS | A | 364 | 120.579 | 92.520 | 55.963 | 1.00 | 39.20 | 7 |
| ATOM | 1558 | C | LYS | A | 364 | 117.741 | 87.062 | 58.525 | 1.00 | 27.98 | 6 |
| ATOM | 1559 | O | LYS | A | 364 | 116.591 | 87.344 | 58.825 | 1.00 | 29.30 | 8 |
| ATOM | 1560 | N | ILE | A | 365 | 118.637 | 86.640 | 59.399 | 1.00 | 29.27 | 7 |
| ATOM | 1561 | CA | ILE | A | 365 | 118.338 | 86.520 | 60.807 | 1.00 | 27.96 | 6 |
| ATOM | 1562 | CB | ILE | A | 365 | 119.054 | 85.308 | 61.413 | 1.00 | 29.40 | 6 |
| ATOM | 1563 | CG2 | ILE | A | 365 | 118.698 | 85.179 | 62.882 | 1.00 | 26.96 | 6 |
| ATOM | 1564 | CG1 | ILE | A | 365 | 118.703 | 84.040 | 60.618 | 1.00 | 27.17 | 6 |
| ATOM | 1565 | CD1 | ILE | A | 365 | 117.230 | 83.699 | 60.586 | 1.00 | 20.52 | 6 |
| ATOM | 1566 | C | ILE | A | 365 | 118.932 | 87.800 | 61.395 | 1.00 | 29.99 | 6 |
| ATOM | 1567 | O | ILE | A | 365 | 120.154 | 87.952 | 61.469 | 1.00 | 30.68 | 8 |
| ATOM | 1568 | N | ARG | A | 366 | 118.076 | 88.736 | 61.778 | 1.00 | 30.08 | 7 |
| ATOM | 1569 | CA | ARG | A | 366 | 118.564 | 89.989 | 62.346 | 1.00 | 31.76 | 6 |
| ATOM | 1570 | CB | ARG | A | 366 | 117.450 | 91.039 | 62.319 | 1.00 | 32.42 | 6 |
| ATOM | 1571 | CG | ARG | A | 366 | 116.968 | 91.327 | 60.888 | 1.00 | 36.06 | 6 |
| ATOM | 1572 | CD | ARG | A | 366 | 115.798 | 92.308 | 60.817 | 1.00 | 41.26 | 6 |
| ATOM | 1573 | NE | ARG | A | 366 | 116.190 | 93.544 | 60.147 | 1.00 | 45.95 | 7 |
| ATOM | 1574 | CZ | ARG | A | 366 | 115.444 | 94.189 | 59.256 | 1.00 | 45.65 | 6 |
| ATOM | 1575 | NH1 | ARG | A | 366 | 114.250 | 93.722 | 58.907 | 1.00 | 44.27 | 7 |
| ATOM | 1576 | NH2 | ARG | A | 366 | 115.896 | 95.310 | 58.714 | 1.00 | 48.75 | 7 |
| ATOM | 1577 | C | ARG | A | 366 | 119.054 | 89.734 | 63.767 | 1.00 | 31.02 | 6 |
| ATOM | 1578 | O | ARG | A | 366 | 120.202 | 90.034 | 64.100 | 1.00 | 32.25 | 8 |
| ATOM | 1579 | N | GLY | A | 367 | 118.197 | 89.178 | 64.596 | 1.00 | 30.10 | 7 |
| ATOM | 1580 | CA | GLY | A | 367 | 118.627 | 88.859 | 65.930 | 1.00 | 28.56 | 6 |
| ATOM | 1581 | C | GLY | A | 367 | 117.463 | 88.303 | 66.735 | 1.00 | 27.96 | 6 |
| ATOM | 1582 | O | GLY | A | 367 | 116.318 | 88.239 | 66.260 | 1.00 | 25.01 | 8 |
| ATOM | 1583 | N | ILE | A | 368 | 117.780 | 87.914 | 67.939 | 1.00 | 28.62 | 7 |
| ATOM | 1584 | CA | ILE | A | 368 | 116.773 | 87.417 | 68.842 | 1.00 | 30.61 | 6 |
| ATOM | 1585 | CB | ILE | A | 368 | 117.103 | 86.020 | 69.378 | 1.00 | 30.60 | 6 |
| ATOM | 1586 | CG2 | ILE | A | 368 | 117.745 | 85.161 | 68.291 | 1.00 | 28.95 | 6 |
| ATOM | 1587 | CG1 | ILE | A | 368 | 118.041 | 86.185 | 70.575 | 1.00 | 30.79 | 6 |
| ATOM | 1588 | CD1 | ILE | A | 368 | 118.404 | 84.887 | 71.251 | 1.00 | 34.89 | 6 |
| ATOM | 1589 | C | ILE | A | 368 | 116.657 | 88.397 | 69.958 | 1.00 | 31.31 | 6 |
| ATOM | 1590 | O | ILE | A | 368 | 117.427 | 89.346 | 70.067 | 1.00 | 31.06 | 8 |
| ATOM | 1591 | N | ASP | A | 369 | 115.677 | 88.141 | 70.817 | 1.00 | 32.79 | 7 |
| ATOM | 1592 | CA | ASP | A | 369 | 115.449 | 89.089 | 71.882 | 1.00 | 35.22 | 6 |
| ATOM | 1593 | CB | ASP | A | 369 | 115.003 | 90.393 | 71.215 | 1.00 | 38.87 | 6 |
| ATOM | 1594 | CG | ASP | A | 369 | 114.768 | 91.503 | 72.180 | 1.00 | 44.73 | 6 |
| ATOM | 1595 | OD1 | ASP | A | 369 | 114.885 | 92.680 | 71.800 | 1.00 | 46.24 | 8 |
| ATOM | 1596 | OD2 | ASP | A | 369 | 114.462 | 91.190 | 73.365 | 1.00 | 45.44 | 8 |
| ATOM | 1597 | C | ASP | A | 369 | 114.470 | 88.625 | 72.965 | 1.00 | 34.43 | 6 |
| ATOM | 1598 | O | ASP | A | 369 | 113.347 | 88.203 | 72.673 | 1.00 | 31.45 | 8 |
| ATOM | 1599 | N | ILE | A | 370 | 114.960 | 88.709 | 74.239 | 1.00 | 35.01 | 7 |
| ATOM | 1600 | CA | ILE | A | 370 | 114.230 | 88.359 | 75.475 | 1.00 | 37.27 | 6 |
| ATOM | 1601 | CB | ILE | A | 370 | 114.564 | 86.923 | 75.936 | 1.00 | 38.85 | 6 |
| ATOM | 1602 | CG2 | ILE | A | 370 | 115.710 | 86.933 | 76.928 | 1.00 | 36.03 | 6 |
| ATOM | 1603 | CG1 | ILE | A | 370 | 113.342 | 86.204 | 76.529 | 1.00 | 41.90 | 6 |
| ATOM | 1604 | CD1 | ILE | A | 370 | 113.626 | 84.793 | 76.983 | 1.00 | 45.17 | 6 |
| ATOM | 1605 | C | ILE | A | 370 | 114.597 | 89.286 | 76.632 | 1.00 | 38.32 | 6 |
| ATOM | 1606 | O | ILE | A | 370 | 115.743 | 89.681 | 76.847 | 1.00 | 35.74 | 8 |
| ATOM | 1607 | N | PRO | A | 371 | 113.527 | 89.603 | 77.371 | 1.00 | 39.96 | 7 |
| ATOM | 1608 | CD | PRO | A | 371 | 112.354 | 89.990 | 76.612 | 1.00 | 41.59 | 6 |
| ATOM | 1609 | CA | PRO | A | 371 | 113.628 | 90.500 | 78.541 | 1.00 | 43.08 | 6 |
| ATOM | 1610 | CB | PRO | A | 371 | 112.158 | 90.662 | 78.932 | 1.00 | 41.63 | 6 |
| ATOM | 1611 | CG | PRO | A | 371 | 111.477 | 90.709 | 77.596 | 1.00 | 42.26 | 6 |
| ATOM | 1612 | C | PRO | A | 371 | 114.586 | 90.083 | 79.647 | 1.00 | 45.58 | 6 |
| ATOM | 1613 | O | PRO | A | 371 | 115.585 | 90.726 | 79.949 | 1.00 | 46.13 | 8 |
| ATOM | 1614 | N | VAL | A | 372 | 114.174 | 88.933 | 80.342 | 1.00 | 48.89 | 7 |
| ATOM | 1615 | CA | VAL | A | 372 | 114.756 | 88.235 | 81.573 | 1.00 | 53.86 | 6 |
| ATOM | 1616 | CB | VAL | A | 372 | 113.615 | 87.914 | 82.553 | 1.00 | 53.36 | 6 |
| ATOM | 1617 | CG1 | VAL | A | 372 | 114.068 | 88.133 | 83.994 | 1.00 | 52.96 | 6 |
| ATOM | 1618 | CG2 | VAL | A | 372 | 112.375 | 88.762 | 82.246 | 1.00 | 53.07 | 6 |
| ATOM | 1619 | C | VAL | A | 372 | 115.461 | 86.906 | 81.301 | 1.00 | 57.32 | 6 |
| ATOM | 1620 | O | VAL | A | 372 | 114.961 | 86.092 | 80.524 | 1.00 | 57.52 | 8 |
| ATOM | 1621 | N | LEU | A | 373 | 116.634 | 86.586 | 81.970 | 1.00 | 61.39 | 7 |
| ATOM | 1622 | CA | LEU | A | 373 | 117.302 | 85.295 | 81.627 | 1.00 | 65.98 | 6 |
| ATOM | 1623 | CB | LEU | A | 373 | 118.033 | 85.504 | 80.327 | 1.00 | 66.33 | 6 |
| ATOM | 1624 | CG | LEU | A | 373 | 117.894 | 84.349 | 79.349 | 1.00 | 66.68 | 6 |
| ATOM | 1625 | CD1 | LEU | A | 373 | 118.007 | 84.848 | 77.922 | 1.00 | 66.83 | 6 |
| ATOM | 1626 | CD2 | LEU | A | 373 | 118.925 | 83.275 | 79.626 | 1.00 | 67.73 | 6 |
| ATOM | 1627 | C | LEU | A | 373 | 118.268 | 84.517 | 82.594 | 1.00 | 68.85 | 6 |
| ATOM | 1628 | O | LEU | A | 373 | 119.487 | 84.553 | 82.403 | 1.00 | 70.08 | 8 |
| ATOM | 1629 | N | PRO | A | 374 | 117.715 | 83.820 | 83.628 | 1.00 | 70.66 | 7 |
| ATOM | 1630 | CD | PRO | A | 374 | 116.863 | 84.557 | 84.590 | 1.00 | 70.73 | 6 |
| ATOM | 1631 | CA | PRO | A | 374 | 118.574 | 82.994 | 84.481 | 1.00 | 72.57 | 6 |
| ATOM | 1632 | CB | PRO | A | 374 | 117.659 | 82.530 | 85.596 | 1.00 | 72.65 | 6 |
| ATOM | 1633 | CG | PRO | A | 374 | 116.831 | 83.735 | 85.842 | 1.00 | 72.24 | 6 |
| ATOM | 1634 | C | PRO | A | 374 | 119.394 | 81.873 | 83.848 | 1.00 | 74.37 | 6 |
| ATOM | 1635 | O | PRO | A | 374 | 119.253 | 80.714 | 84.239 | 1.00 | 74.01 | 8 |
| ATOM | 1636 | N | LEU | A | 379 | 125.852 | 80.376 | 79.051 | 1.00 | 70.76 | 7 |
| ATOM | 1637 | CA | LEU | A | 379 | 125.910 | 79.427 | 77.945 | 1.00 | 67.57 | 6 |
| ATOM | 1638 | CB | LEU | A | 379 | 124.700 | 78.480 | 77.977 | 1.00 | 68.98 | 6 |
| ATOM | 1639 | CG | LEU | A | 379 | 125.018 | 76.977 | 78.094 | 1.00 | 69.23 | 6 |
| ATOM | 1640 | CD1 | LEU | A | 379 | 123.846 | 76.153 | 77.581 | 1.00 | 68.26 | 6 |
| ATOM | 1641 | CD2 | LEU | A | 379 | 126.294 | 76.623 | 77.330 | 1.00 | 69.30 | 6 |
| ATOM | 1642 | C | LEU | A | 379 | 125.879 | 80.290 | 76.704 | 1.00 | 64.19 | 6 |
| ATOM | 1643 | O | LEU | A | 379 | 125.563 | 81.464 | 76.812 | 1.00 | 62.46 | 8 |
| ATOM | 1644 | N | THR | A | 380 | 126.168 | 79.780 | 75.556 | 1.00 | 60.90 | 7 |
| ATOM | 1645 | CA | THR | A | 380 | 126.059 | 80.712 | 74.441 | 1.00 | 56.99 | 6 |
| ATOM | 1646 | CB | THR | A | 380 | 127.293 | 80.607 | 73.549 | 1.00 | 57.62 | 6 |
| ATOM | 1647 | OG1 | THR | A | 380 | 127.314 | 81.702 | 72.636 | 1.00 | 59.02 | 8 |
| ATOM | 1648 | CG2 | THR | A | 380 | 127.266 | 79.310 | 72.768 | 1.00 | 57.22 | 6 |
| ATOM | 1649 | C | THR | A | 380 | 124.716 | 80.465 | 73.714 | 1.00 | 53.64 | 6 |
| ATOM | 1650 | O | THR | A | 380 | 123.714 | 80.286 | 74.412 | 1.00 | 52.87 | 8 |
| ATOM | 1651 | N | VAL | A | 381 | 124.647 | 80.448 | 72.371 | 1.00 | 49.68 | 7 |
| ATOM | 1652 | CA | VAL | A | 381 | 123.384 | 80.225 | 71.649 | 1.00 | 46.32 | 6 |
| ATOM | 1653 | CB | VAL | A | 381 | 122.244 | 81.114 | 72.197 | 1.00 | 46.20 | 6 |
| ATOM | 1654 | CG1 | VAL | A | 381 | 121.275 | 81.493 | 71.081 | 1.00 | 47.99 | 6 |
| ATOM | 1655 | CG2 | VAL | A | 381 | 121.513 | 80.423 | 73.340 | 1.00 | 45.82 | 6 |
| ATOM | 1656 | C | VAL | A | 381 | 123.523 | 80.553 | 70.139 | 1.00 | 42.87 | 6 |
| ATOM | 1657 | O | VAL | A | 381 | 124.247 | 81.478 | 69.776 | 1.00 | 40.64 | 8 |
| ATOM | 1658 | N | PHE | A | 382 | 122.831 | 79.802 | 69.291 | 1.00 | 40.36 | 7 |
| ATOM | 1659 | CA | PHE | A | 382 | 122.857 | 80.050 | 67.856 | 1.00 | 38.03 | 6 |
| ATOM | 1660 | CB | PHE | A | 382 | 123.894 | 79.171 | 67.134 | 1.00 | 37.65 | 6 |
| ATOM | 1661 | CG | PHE | A | 382 | 123.518 | 77.722 | 67.034 | 1.00 | 38.05 | 6 |
| ATOM | 1662 | CD1 | PHE | A | 382 | 123.868 | 76.826 | 68.040 | 1.00 | 39.20 | 6 |
| ATOM | 1663 | CD2 | PHE | A | 382 | 122.830 | 77.245 | 65.926 | 1.00 | 37.75 | 6 |
| ATOM | 1664 | CE1 | PHE | A | 382 | 123.538 | 75.470 | 67.937 | 1.00 | 38.75 | 6 |
| ATOM | 1665 | CE2 | PHE | A | 382 | 122.497 | 75.895 | 65.814 | 1.00 | 38.59 | 6 |
| ATOM | 1666 | CZ | PHE | A | 382 | 122.851 | 75.006 | 66.818 | 1.00 | 37.07 | 6 |
| ATOM | 1667 | C | PHE | A | 382 | 121.458 | 79.783 | 67.300 | 1.00 | 36.13 | 6 |
| ATOM | 1668 | O | PHE | A | 382 | 120.610 | 79.201 | 67.980 | 1.00 | 35.72 | 8 |
| ATOM | 1669 | N | VAL | A | 383 | 121.210 | 80.229 | 66.076 | 1.00 | 33.99 | 7 |
| ATOM | 1670 | CA | VAL | A | 383 | 119.910 | 80.025 | 65.461 | 1.00 | 32.05 | 6 |
| ATOM | 1671 | CB | VAL | A | 383 | 119.338 | 81.345 | 64.896 | 1.00 | 31.18 | 6 |
| ATOM | 1672 | CG1 | VAL | A | 383 | 118.195 | 81.060 | 63.932 | 1.00 | 31.69 | 6 |
| ATOM | 1673 | CG2 | VAL | A | 383 | 118.840 | 82.219 | 66.035 | 1.00 | 33.40 | 6 |
| ATOM | 1674 | C | VAL | A | 383 | 119.987 | 79.002 | 64.334 | 1.00 | 31.98 | 6 |
| ATOM | 1675 | O | VAL | A | 383 | 120.869 | 79.064 | 63.475 | 1.00 | 31.58 | 8 |
| ATOM | 1676 | N | GLU | A | 384 | 119.058 | 78.057 | 64.358 | 1.00 | 31.24 | 7 |
| ATOM | 1677 | CA | GLU | A | 384 | 118.969 | 77.037 | 63.328 | 1.00 | 31.91 | 6 |
| ATOM | 1678 | CB | GLU | A | 384 | 118.814 | 75.656 | 63.962 | 1.00 | 33.82 | 6 |
| ATOM | 1679 | CG | GLU | A | 384 | 118.062 | 74.656 | 63.086 | 1.00 | 37.35 | 6 |
| ATOM | 1680 | CD | GLU | A | 384 | 118.373 | 73.218 | 63.459 | 1.00 | 39.05 | 6 |
| ATOM | 1681 | OE1 | GLU | A | 384 | 117.426 | 72.408 | 63.621 | 1.00 | 38.26 | 8 |
| ATOM | 1682 | OE2 | GLU | A | 384 | 119.572 | 72.905 | 63.604 | 1.00 | 39.27 | 8 |
| ATOM | 1683 | C | GLU | A | 384 | 117.747 | 77.325 | 62.457 | 1.00 | 30.72 | 6 |
| ATOM | 1684 | O | GLU | A | 384 | 116.632 | 77.448 | 62.963 | 1.00 | 30.78 | 8 |
| ATOM | 1685 | N | ALA | A | 385 | 117.958 | 77.441 | 61.152 | 1.00 | 30.13 | 7 |
| ATOM | 1686 | CA | ALA | A | 385 | 116.860 | 77.689 | 60.227 | 1.00 | 28.42 | 6 |
| ATOM | 1687 | CB | ALA | A | 385 | 117.171 | 78.899 | 59.368 | 1.00 | 27.92 | 6 |
| ATOM | 1688 | C | ALA | A | 385 | 116.684 | 76.450 | 59.351 | 1.00 | 28.43 | 6 |
| ATOM | 1689 | O | ALA | A | 385 | 117.615 | 76.034 | 58.670 | 1.00 | 29.37 | 8 |
| ATOM | 1690 | N | ASN | A | 386 | 115.490 | 75.867 | 59.372 | 1.00 | 29.34 | 7 |
| ATOM | 1691 | CA | ASN | A | 386 | 115.191 | 74.668 | 58.591 | 1.00 | 28.74 | 6 |
| ATOM | 1692 | CB | ASN | A | 386 | 114.808 | 73.495 | 59.510 | 1.00 | 28.36 | 6 |
| ATOM | 1693 | CG | ASN | A | 386 | 115.867 | 73.178 | 60.542 | 1.00 | 29.12 | 6 |
| ATOM | 1694 | OD1 | ASN | A | 386 | 116.983 | 72.773 | 60.209 | 1.00 | 27.30 | 8 |
| ATOM | 1695 | ND2 | ASN | A | 386 | 115.525 | 73.366 | 61.808 | 1.00 | 30.29 | 7 |
| ATOM | 1696 | C | ASN | A | 386 | 114.016 | 74.900 | 57.660 | 1.00 | 28.79 | 6 |
| ATOM | 1697 | O | ASN | A | 386 | 113.052 | 75.586 | 58.030 | 1.00 | 29.45 | 8 |
| ATOM | 1698 | N | ILE | A | 387 | 114.120 | 74.367 | 56.457 | 1.00 | 27.43 | 7 |
| ATOM | 1699 | CA | ILE | A | 387 | 113.006 | 74.430 | 55.515 | 1.00 | 27.21 | 6 |
| ATOM | 1700 | CB | ILE | A | 387 | 113.435 | 74.626 | 54.056 | 1.00 | 26.51 | 6 |
| ATOM | 1701 | CG2 | ILE | A | 387 | 112.221 | 74.764 | 53.161 | 1.00 | 26.17 | 6 |
| ATOM | 1702 | CG1 | ILE | A | 387 | 114.338 | 75.861 | 53.917 | 1.00 | 29.16 | 6 |
| ATOM | 1703 | CD1 | ILE | A | 387 | 113.643 | 77.175 | 54.213 | 1.00 | 32.00 | 6 |
| ATOM | 1704 | C | ILE | A | 387 | 112.352 | 73.091 | 55.654 | 1.00 | 30.82 | 6 |
| ATOM | 1705 | O | ILE | A | 387 | 112.972 | 72.056 | 55.460 | 1.00 | 30.07 | 8 |
| ATOM | 1706 | N | GLN | A | 388 | 111.092 | 73.068 | 55.996 | 1.00 | 30.30 | 7 |
| ATOM | 1707 | CA | GLN | A | 388 | 110.508 | 71.775 | 56.198 | 1.00 | 30.67 | 6 |
| ATOM | 1708 | CB | GLN | A | 388 | 110.180 | 71.563 | 57.658 | 1.00 | 31.79 | 6 |
| ATOM | 1709 | CG | GLN | A | 388 | 111.354 | 71.455 | 58.573 | 1.00 | 37.81 | 6 |
| ATOM | 1710 | CD | GLN | A | 388 | 110.834 | 71.422 | 59.981 | 1.00 | 40.42 | 6 |
| ATOM | 1711 | OE1 | GLN | A | 388 | 109.785 | 70.822 | 60.251 | 1.00 | 44.02 | 8 |
| ATOM | 1712 | NE2 | GLN | A | 388 | 111.391 | 71.986 | 61.056 | 1.00 | 38.20 | 7 |
| ATOM | 1713 | C | GLN | A | 388 | 109.212 | 71.541 | 55.538 | 1.00 | 30.14 | 6 |
| ATOM | 1714 | O | GLN | A | 388 | 108.450 | 72.469 | 55.265 | 1.00 | 30.76 | 8 |
| ATOM | 1715 | N | HIS | A | 389 | 108.940 | 70.302 | 55.232 | 1.00 | 30.27 | 7 |
| ATOM | 1716 | CA | HIS | A | 389 | 107.650 | 70.074 | 54.699 | 1.00 | 30.60 | 6 |
| ATOM | 1717 | CB | HIS | A | 389 | 107.658 | 69.573 | 53.299 | 1.00 | 28.19 | 6 |
| ATOM | 1718 | CG | HIS | A | 389 | 106.352 | 70.080 | 52.657 | 1.00 | 25.37 | 6 |
| ATOM | 1719 | CD2 | HIS | A | 389 | 106.116 | 71.228 | 51.980 | 1.00 | 26.46 | 6 |
| ATOM | 1720 | ND1 | HIS | A | 389 | 105.163 | 69.381 | 52.677 | 1.00 | 26.07 | 7 |
| ATOM | 1721 | CE1 | HIS | A | 389 | 104.253 | 70.072 | 52.045 | 1.00 | 27.68 | 6 |
| ATOM | 1722 | NE2 | HIS | A | 389 | 104.805 | 71.196 | 51.598 | 1.00 | 28.24 | 7 |
| ATOM | 1723 | C | HIS | A | 389 | 106.902 | 69.118 | 55.566 | 1.00 | 32.78 | 6 |
| ATOM | 1724 | O | HIS | A | 389 | 107.334 | 67.990 | 55.770 | 1.00 | 30.39 | 8 |
| ATOM | 1725 | N | GLY | A | 390 | 105.776 | 69.557 | 56.067 | 1.00 | 35.56 | 7 |
| ATOM | 1726 | CA | GLY | A | 390 | 105.201 | 68.680 | 57.000 | 1.00 | 38.01 | 6 |
| ATOM | 1727 | C | GLY | A | 390 | 106.420 | 68.450 | 57.847 | 1.00 | 41.02 | 6 |
| ATOM | 1728 | O | GLY | A | 390 | 107.028 | 69.421 | 58.304 | 1.00 | 40.60 | 8 |
| ATOM | 1729 | N | GLN | A | 391 | 106.786 | 67.263 | 58.132 | 1.00 | 43.40 | 7 |
| ATOM | 1730 | CA | GLN | A | 391 | 107.975 | 67.225 | 58.950 | 1.00 | 46.51 | 6 |
| ATOM | 1731 | CB | GLN | A | 391 | 107.657 | 66.763 | 60.363 | 1.00 | 48.37 | 6 |
| ATOM | 1732 | CG | GLN | A | 391 | 108.243 | 65.451 | 60.804 | 1.00 | 50.47 | 6 |
| ATOM | 1733 | CD | GLN | A | 391 | 107.373 | 64.832 | 61.863 | 1.00 | 51.81 | 6 |
| ATOM | 1734 | OE1 | GLN | A | 391 | 106.131 | 64.863 | 61.773 | 1.00 | 52.47 | 8 |
| ATOM | 1735 | NE2 | GLN | A | 391 | 107.802 | 64.209 | 62.960 | 1.00 | 53.23 | 7 |
| ATOM | 1736 | C | GLN | A | 391 | 109.008 | 66.432 | 58.214 | 1.00 | 48.25 | 6 |
| ATOM | 1737 | O | GLN | A | 391 | 109.019 | 65.190 | 58.254 | 1.00 | 49.70 | 8 |
| ATOM | 1738 | N | GLN | A | 392 | 109.880 | 67.169 | 57.546 | 1.00 | 49.28 | 7 |
| ATOM | 1739 | CA | GLN | A | 392 | 110.924 | 66.621 | 56.735 | 1.00 | 48.95 | 6 |
| ATOM | 1740 | CB | GLN | A | 392 | 110.326 | 65.774 | 55.615 | 1.00 | 50.57 | 6 |
| ATOM | 1741 | CG | GLN | A | 392 | 110.730 | 66.192 | 54.225 | 1.00 | 52.12 | 6 |
| ATOM | 1742 | CD | GLN | A | 392 | 109.666 | 65.874 | 53.175 | 1.00 | 54.36 | 6 |
| ATOM | 1743 | OE1 | GLN | A | 392 | 108.941 | 66.762 | 52.730 | 1.00 | 55.58 | 8 |
| ATOM | 1744 | NE2 | GLN | A | 392 | 109.420 | 64.675 | 52.657 | 1.00 | 55.46 | 7 |
| ATOM | 1745 | C | GLN | A | 392 | 111.774 | 67.748 | 56.193 | 1.00 | 47.23 | 6 |
| ATOM | 1746 | O | GLN | A | 392 | 111.452 | 68.338 | 55.167 | 1.00 | 45.76 | 8 |
| ATOM | 1747 | N | VAL | A | 393 | 112.871 | 68.052 | 56.893 | 1.00 | 45.37 | 7 |
| ATOM | 1748 | CA | VAL | A | 393 | 113.758 | 69.159 | 56.572 | 1.00 | 43.58 | 6 |
| ATOM | 1749 | CB | VAL | A | 393 | 114.556 | 69.515 | 57.832 | 1.00 | 44.03 | 6 |
| ATOM | 1750 | CG1 | VAL | A | 393 | 113.617 | 69.951 | 58.950 | 1.00 | 43.96 | 6 |
| ATOM | 1751 | CG2 | VAL | A | 393 | 115.434 | 68.352 | 58.278 | 1.00 | 45.17 | 6 |
| ATOM | 1752 | C | VAL | A | 393 | 114.622 | 68.974 | 55.323 | 1.00 | 41.16 | 6 |
| ATOM | 1753 | O | VAL | A | 393 | 115.622 | 68.269 | 55.309 | 1.00 | 41.10 | 8 |
| ATOM | 1754 | N | LEU | A | 394 | 114.147 | 69.659 | 54.256 | 1.00 | 39.17 | 7 |
| ATOM | 1755 | CA | LEU | A | 394 | 114.723 | 69.708 | 52.896 | 1.00 | 38.62 | 6 |
| ATOM | 1756 | CB | LEU | A | 394 | 113.703 | 70.307 | 51.928 | 1.00 | 37.59 | 6 |
| ATOM | 1757 | CG | LEU | A | 394 | 112.316 | 70.509 | 52.493 | 1.00 | 36.77 | 6 |
| ATOM | 1758 | CD1 | LEU | A | 394 | 111.440 | 71.303 | 51.542 | 1.00 | 35.32 | 6 |
| ATOM | 1759 | CD2 | LEU | A | 394 | 111.676 | 69.175 | 52.814 | 1.00 | 36.94 | 6 |
| ATOM | 1760 | C | LEU | A | 394 | 116.028 | 70.483 | 52.857 | 1.00 | 40.82 | 6 |
| ATOM | 1761 | O | LEU | A | 394 | 116.926 | 70.159 | 52.081 | 1.00 | 40.75 | 8 |
| ATOM | 1762 | N | CYS | A | 395 | 116.157 | 71.516 | 53.701 | 1.00 | 39.53 | 7 |
| ATOM | 1763 | CA | CYS | A | 395 | 117.378 | 72.337 | 53.771 | 1.00 | 39.94 | 6 |
| ATOM | 1764 | CB | CYS | A | 395 | 117.305 | 73.510 | 52.785 | 1.00 | 41.74 | 6 |
| ATOM | 1765 | SG | CYS | A | 395 | 118.855 | 74.445 | 52.597 | 1.00 | 52.80 | 16 |
| ATOM | 1766 | C | CYS | A | 395 | 117.595 | 72.817 | 55.178 | 1.00 | 37.07 | 6 |
| ATOM | 1767 | O | CYS | A | 395 | 116.680 | 72.867 | 56.005 | 1.00 | 35.84 | 8 |
| ATOM | 1768 | N | GLN | A | 396 | 118.845 | 73.175 | 55.468 | 1.00 | 35.22 | 7 |
| ATOM | 1769 | CA | GLN | A | 396 | 119.196 | 73.573 | 56.821 | 1.00 | 35.93 | 6 |
| ATOM | 1770 | CB | GLN | A | 396 | 119.464 | 72.326 | 57.643 | 1.00 | 38.55 | 6 |
| ATOM | 1771 | CG | GLN | A | 396 | 119.684 | 72.569 | 59.124 | 1.00 | 41.41 | 6 |
| ATOM | 1772 | CD | GLN | A | 396 | 119.848 | 71.278 | 59.898 | 1.00 | 46.21 | 6 |
| ATOM | 1773 | OE1 | GLN | A | 396 | 120.849 | 70.573 | 59.739 | 1.00 | 48.98 | 8 |
| ATOM | 1774 | NE2 | GLN | A | 396 | 118.996 | 70.759 | 60.783 | 1.00 | 44.86 | 7 |
| ATOM | 1775 | C | GLN | A | 396 | 120.408 | 74.468 | 56.863 | 1.00 | 36.84 | 6 |
| ATOM | 1776 | O | GLN | A | 396 | 121.418 | 74.200 | 56.236 | 1.00 | 35.42 | 8 |
| ATOM | 1777 | N | ARG | A | 397 | 120.272 | 75.554 | 57.617 | 1.00 | 35.45 | 7 |
| ATOM | 1778 | CA | ARG | A | 397 | 121.361 | 76.512 | 57.755 | 1.00 | 34.90 | 6 |
| ATOM | 1779 | CB | ARG | A | 397 | 121.161 | 77.711 | 56.834 | 1.00 | 34.19 | 6 |
| ATOM | 1780 | CG | ARG | A | 397 | 121.377 | 77.394 | 55.362 | 1.00 | 36.90 | 6 |
| ATOM | 1781 | CD | ARG | A | 397 | 122.816 | 77.021 | 55.082 | 1.00 | 37.11 | 6 |
| ATOM | 1782 | NE | ARG | A | 397 | 123.025 | 76.637 | 53.696 | 1.00 | 41.65 | 7 |
| ATOM | 1783 | CZ | ARG | A | 397 | 122.695 | 75.444 | 53.199 | 1.00 | 41.03 | 6 |
| ATOM | 1784 | NH1 | ARG | A | 397 | 122.139 | 74.521 | 53.968 | 1.00 | 41.60 | 7 |
| ATOM | 1785 | NH2 | ARG | A | 397 | 122.936 | 75.181 | 51.919 | 1.00 | 43.71 | 7 |
| ATOM | 1786 | C | ARG | A | 397 | 121.433 | 76.961 | 59.215 | 1.00 | 35.76 | 6 |
| ATOM | 1787 | O | ARG | A | 397 | 120.413 | 77.011 | 59.913 | 1.00 | 34.91 | 8 |
| ATOM | 1788 | N | ARG | A | 398 | 122.633 | 77.300 | 59.678 | 1.00 | 34.74 | 7 |
| ATOM | 1789 | CA | ARG | A | 398 | 122.832 | 77.746 | 61.056 | 1.00 | 37.25 | 6 |
| ATOM | 1790 | CB | ARG | A | 398 | 123.533 | 76.658 | 61.876 | 1.00 | 38.07 | 6 |
| ATOM | 1791 | CG | ARG | A | 398 | 122.731 | 75.391 | 62.105 | 1.00 | 41.08 | 6 |
| ATOM | 1792 | CD | ARG | A | 398 | 123.521 | 74.400 | 62.957 | 1.00 | 43.72 | 6 |
| ATOM | 1793 | NE | ARG | A | 398 | 122.644 | 73.464 | 63.657 | 1.00 | 48.05 | 7 |
| ATOM | 1794 | CZ | ARG | A | 398 | 123.064 | 72.526 | 64.505 | 1.00 | 49.64 | 6 |
| ATOM | 1795 | NH1 | ARG | A | 398 | 124.360 | 72.386 | 64.760 | 1.00 | 50.42 | 7 |
| ATOM | 1796 | NH2 | ARG | A | 398 | 122.185 | 71.746 | 65.124 | 1.00 | 49.74 | 7 |
| ATOM | 1797 | C | ARG | A | 398 | 123.677 | 79.019 | 61.121 | 1.00 | 38.18 | 6 |
| ATOM | 1798 | O | ARG | A | 398 | 124.371 | 79.364 | 60.162 | 1.00 | 37.91 | 8 |
| ATOM | 1799 | N | THR | A | 399 | 123.600 | 79.714 | 62.253 | 1.00 | 38.85 | 7 |
| ATOM | 1800 | CA | THR | A | 399 | 124.385 | 80.929 | 62.484 | 1.00 | 38.12 | 6 |
| ATOM | 1801 | CB | THR | A | 399 | 123.580 | 82.041 | 63.207 | 1.00 | 37.52 | 6 |
| ATOM | 1802 | OG1 | THR | A | 399 | 123.278 | 81.609 | 64.543 | 1.00 | 36.49 | 8 |
| ATOM | 1803 | CG2 | THR | A | 399 | 122.290 | 82.360 | 62.465 | 1.00 | 34.49 | 6 |
| ATOM | 1804 | C | THR | A | 399 | 125.505 | 80.536 | 63.442 | 1.00 | 39.63 | 6 |
| ATOM | 1805 | O | THR | A | 399 | 125.491 | 79.446 | 64.009 | 1.00 | 38.64 | 8 |
| ATOM | 1806 | N | SER | A | 400 | 126.394 | 81.470 | 63.668 | 1.00 | 41.50 | 7 |
| ATOM | 1807 | CA | SER | A | 400 | 127.499 | 81.300 | 64.585 | 1.00 | 43.23 | 6 |
| ATOM | 1808 | CB | SER | A | 400 | 128.623 | 82.307 | 64.287 | 1.00 | 44.49 | 6 |
| ATOM | 1809 | OG | SER | A | 400 | 128.117 | 83.618 | 64.133 | 1.00 | 47.82 | 8 |
| ATOM | 1810 | C | SER | A | 400 | 127.046 | 81.478 | 66.051 | 1.00 | 44.15 | 6 |
| ATOM | 1811 | O | SER | A | 400 | 126.212 | 82.314 | 66.387 | 1.00 | 44.13 | 8 |
| ATOM | 1812 | N | PRO | A | 401 | 127.674 | 80.621 | 66.890 | 1.00 | 45.15 | 7 |
| ATOM | 1813 | CD | PRO | A | 401 | 127.723 | 79.218 | 66.449 | 1.00 | 46.04 | 6 |
| ATOM | 1814 | CA | PRO | A | 401 | 127.425 | 80.603 | 68.361 | 1.00 | 45.95 | 6 |
| ATOM | 1815 | CB | PRO | A | 401 | 128.236 | 79.435 | 68.855 | 1.00 | 46.61 | 6 |
| ATOM | 1816 | CG | PRO | A | 401 | 128.048 | 78.475 | 67.719 | 1.00 | 46.93 | 6 |
| ATOM | 1817 | C | PRO | A | 401 | 127.718 | 81.942 | 69.000 | 1.00 | 46.44 | 6 |
| ATOM | 1818 | O | PRO | A | 401 | 128.783 | 82.496 | 68.751 | 1.00 | 47.15 | 8 |
| ATOM | 1819 | N | LYS | A | 402 | 126.816 | 82.413 | 69.839 | 1.00 | 46.22 | 7 |
| ATOM | 1820 | CA | LYS | A | 402 | 127.087 | 83.695 | 70.440 | 1.00 | 46.23 | 6 |
| ATOM | 1821 | CB | LYS | A | 402 | 126.356 | 84.811 | 69.699 | 1.00 | 43.82 | 6 |
| ATOM | 1822 | CG | LYS | A | 402 | 127.013 | 85.235 | 68.398 | 1.00 | 44.50 | 6 |
| ATOM | 1823 | CD | LYS | A | 402 | 126.507 | 86.588 | 67.930 | 1.00 | 43.79 | 6 |
| ATOM | 1824 | CE | LYS | A | 402 | 127.343 | 87.120 | 66.770 | 1.00 | 43.04 | 6 |
| ATOM | 1825 | NZ | LYS | A | 402 | 126.653 | 88.220 | 66.050 | 1.00 | 43.86 | 7 |
| ATOM | 1826 | C | LYS | A | 402 | 126.687 | 83.774 | 71.876 | 1.00 | 46.53 | 6 |
| ATOM | 1827 | O | LYS | A | 402 | 126.202 | 82.832 | 72.478 | 1.00 | 47.86 | 8 |
| ATOM | 1828 | N | PRO | A | 403 | 126.916 | 84.969 | 72.388 | 1.00 | 46.77 | 7 |
| ATOM | 1829 | CD | PRO | A | 403 | 128.230 | 85.487 | 72.078 | 1.00 | 44.23 | 6 |
| ATOM | 1830 | CA | PRO | A | 403 | 126.562 | 85.274 | 73.780 | 1.00 | 45.45 | 6 |
| ATOM | 1831 | CB | PRO | A | 403 | 127.165 | 86.668 | 73.937 | 1.00 | 45.28 | 6 |
| ATOM | 1832 | CG | PRO | A | 403 | 128.410 | 86.565 | 73.108 | 1.00 | 44.61 | 6 |
| ATOM | 1833 | C | PRO | A | 403 | 125.100 | 85.105 | 74.182 | 1.00 | 44.00 | 6 |
| ATOM | 1834 | O | PRO | A | 403 | 124.249 | 85.785 | 73.602 | 1.00 | 42.44 | 8 |
| ATOM | 1835 | N | PHE | A | 404 | 124.744 | 84.267 | 75.121 | 1.00 | 43.96 | 7 |
| ATOM | 1836 | CA | PHE | A | 404 | 123.313 | 84.318 | 75.410 | 1.00 | 43.12 | 6 |
| ATOM | 1837 | CB | PHE | A | 404 | 122.736 | 83.017 | 75.981 | 1.00 | 42.95 | 6 |
| ATOM | 1838 | CG | PHE | A | 404 | 121.226 | 82.983 | 75.922 | 1.00 | 41.24 | 6 |
| ATOM | 1839 | CD1 | PHE | A | 404 | 120.498 | 83.924 | 75.194 | 1.00 | 42.33 | 6 |
| ATOM | 1840 | CD2 | PHE | A | 404 | 120.532 | 81.992 | 76.614 | 1.00 | 41.66 | 6 |
| ATOM | 1841 | CE1 | PHE | A | 404 | 119.103 | 83.884 | 75.156 | 1.00 | 40.05 | 6 |
| ATOM | 1842 | CE2 | PHE | A | 404 | 119.133 | 81.940 | 76.582 | 1.00 | 41.22 | 6 |
| ATOM | 1843 | CZ | PHE | A | 404 | 118.417 | 82.892 | 75.850 | 1.00 | 42.11 | 6 |
| ATOM | 1844 | C | PHE | A | 404 | 123.018 | 85.486 | 76.332 | 1.00 | 42.88 | 6 |
| ATOM | 1845 | O | PHE | A | 404 | 123.128 | 85.370 | 77.553 | 1.00 | 43.09 | 8 |
| ATOM | 1846 | N | THR | A | 405 | 122.645 | 86.613 | 75.728 | 1.00 | 43.06 | 7 |
| ATOM | 1847 | CA | THR | A | 405 | 122.267 | 87.829 | 76.454 | 1.00 | 42.09 | 6 |
| ATOM | 1848 | CB | THR | A | 405 | 123.201 | 89.020 | 76.127 | 1.00 | 42.12 | 6 |
| ATOM | 1849 | OG1 | THR | A | 405 | 123.041 | 89.396 | 74.751 | 1.00 | 41.22 | 8 |
| ATOM | 1850 | CG2 | THR | A | 405 | 124.658 | 88.646 | 76.395 | 1.00 | 41.76 | 6 |
| ATOM | 1851 | C | THR | A | 405 | 120.845 | 88.221 | 76.036 | 1.00 | 42.13 | 6 |
| ATOM | 1852 | O | THR | A | 405 | 120.341 | 87.752 | 75.016 | 1.00 | 41.19 | 8 |
| ATOM | 1853 | N | GLU | A | 406 | 120.215 | 89.091 | 76.823 | 1.00 | 40.87 | 7 |
| ATOM | 1854 | CA | GLU | A | 406 | 118.851 | 89.547 | 76.561 | 1.00 | 40.98 | 6 |
| ATOM | 1855 | CB | GLU | A | 406 | 118.527 | 90.718 | 77.484 | 1.00 | 43.33 | 6 |
| ATOM | 1856 | CG | GLU | A | 406 | 119.006 | 90.453 | 78.905 | 1.00 | 48.92 | 6 |
| ATOM | 1857 | CD | GLU | A | 406 | 118.657 | 91.566 | 79.861 | 1.00 | 51.91 | 6 |
| ATOM | 1858 | OE1 | GLU | A | 406 | 119.038 | 92.725 | 79.587 | 1.00 | 54.04 | 8 |
| ATOM | 1859 | OE2 | GLU | A | 406 | 118.007 | 91.272 | 80.889 | 1.00 | 53.97 | 8 |
| ATOM | 1860 | C | GLU | A | 406 | 118.643 | 89.933 | 75.097 | 1.00 | 39.83 | 6 |
| ATOM | 1861 | O | GLU | A | 406 | 117.528 | 89.866 | 74.579 | 1.00 | 40.00 | 8 |
| ATOM | 1862 | N | GLU | A | 407 | 119.710 | 90.351 | 74.433 | 1.00 | 37.21 | 7 |
| ATOM | 1863 | CA | GLU | A | 407 | 119.611 | 90.678 | 73.022 | 1.00 | 37.87 | 6 |
| ATOM | 1864 | CB | GLU | A | 407 | 119.462 | 92.184 | 72.797 | 1.00 | 38.69 | 6 |
| ATOM | 1865 | CG | GLU | A | 407 | 119.257 | 92.531 | 71.319 | 1.00 | 39.28 | 6 |
| ATOM | 1866 | CD | GLU | A | 407 | 119.528 | 93.990 | 70.995 | 1.00 | 40.65 | 6 |
| ATOM | 1867 | OE1 | GLU | A | 407 | 120.611 | 94.499 | 71.364 | 1.00 | 40.39 | 8 |
| ATOM | 1868 | OE2 | GLU | A | 407 | 118.663 | 94.626 | 70.359 | 1.00 | 41.98 | 8 |
| ATOM | 1869 | C | GLU | A | 407 | 120.856 | 90.173 | 72.294 | 1.00 | 38.31 | 6 |
| ATOM | 1870 | O | GLU | A | 407 | 121.982 | 90.521 | 72.652 | 1.00 | 37.11 | 8 |
| ATOM | 1871 | N | VAL | A | 408 | 120.642 | 89.331 | 71.288 | 1.00 | 37.85 | 7 |
| ATOM | 1872 | CA | VAL | A | 408 | 121.733 | 88.781 | 70.492 | 1.00 | 37.42 | 6 |
| ATOM | 1873 | CB | VAL | A | 408 | 121.811 | 87.252 | 70.604 | 1.00 | 37.35 | 6 |
| ATOM | 1874 | CG1 | VAL | A | 408 | 122.957 | 86.737 | 69.752 | 1.00 | 36.54 | 6 |
| ATOM | 1875 | CG2 | VAL | A | 408 | 121.988 | 86.845 | 72.051 | 1.00 | 36.25 | 6 |
| ATOM | 1876 | C | VAL | A | 408 | 121.480 | 89.133 | 69.038 | 1.00 | 38.09 | 6 |
| ATOM | 1877 | O | VAL | A | 408 | 120.413 | 88.844 | 68.506 | 1.00 | 36.33 | 8 |
| ATOM | 1878 | N | LEU | A | 409 | 122.471 | 89.752 | 68.401 | 1.00 | 39.25 | 7 |
| ATOM | 1879 | CA | LEU | A | 409 | 122.350 | 90.166 | 67.012 | 1.00 | 40.24 | 6 |
| ATOM | 1880 | CB | LEU | A | 409 | 122.640 | 91.665 | 66.889 | 1.00 | 41.40 | 6 |
| ATOM | 1881 | CG | LEU | A | 409 | 121.760 | 92.653 | 67.667 | 1.00 | 42.57 | 6 |
| ATOM | 1882 | CD1 | LEU | A | 409 | 122.310 | 94.062 | 67.555 | 1.00 | 41.86 | 6 |
| ATOM | 1883 | CD2 | LEU | A | 409 | 120.318 | 92.609 | 67.173 | 1.00 | 42.68 | 6 |
| ATOM | 1884 | C | LEU | A | 409 | 123.275 | 89.392 | 66.072 | 1.00 | 40.68 | 6 |
| ATOM | 1885 | O | LEU | A | 409 | 124.322 | 88.915 | 66.473 | 1.00 | 40.72 | 8 |
| ATOM | 1886 | N | TRP | A | 410 | 122.839 | 89.292 | 64.840 | 1.00 | 40.78 | 7 |
| ATOM | 1887 | CA | TRP | A | 410 | 123.626 | 88.600 | 63.867 | 1.00 | 39.67 | 6 |
| ATOM | 1888 | CB | TRP | A | 410 | 123.036 | 87.237 | 63.547 | 1.00 | 38.34 | 6 |
| ATOM | 1889 | CG | TRP | A | 410 | 123.524 | 86.178 | 64.454 | 1.00 | 35.21 | 6 |
| ATOM | 1890 | CD2 | TRP | A | 410 | 122.808 | 85.616 | 65.560 | 1.00 | 35.72 | 6 |
| ATOM | 1891 | CE2 | TRP | A | 410 | 123.658 | 84.659 | 66.163 | 1.00 | 34.68 | 6 |
| ATOM | 1892 | CE3 | TRP | A | 410 | 121.534 | 85.828 | 66.103 | 1.00 | 35.54 | 6 |
| ATOM | 1893 | CD1 | TRP | A | 410 | 124.740 | 85.560 | 64.430 | 1.00 | 34.64 | 6 |
| ATOM | 1894 | NE1 | TRP | A | 410 | 124.828 | 84.646 | 65.452 | 1.00 | 35.22 | 7 |
| ATOM | 1895 | CZ2 | TRP | A | 410 | 123.274 | 83.912 | 67.285 | 1.00 | 35.15 | 6 |
| ATOM | 1896 | CZ3 | TRP | A | 410 | 121.152 | 85.085 | 67.222 | 1.00 | 36.56 | 6 |
| ATOM | 1897 | CH2 | TRP | A | 410 | 122.022 | 84.138 | 67.801 | 1.00 | 35.91 | 6 |
| ATOM | 1898 | C | TRP | A | 410 | 123.594 | 89.432 | 62.637 | 1.00 | 39.35 | 6 |
| ATOM | 1899 | O | TRP | A | 410 | 124.597 | 89.960 | 62.168 | 1.00 | 39.93 | 8 |
| ATOM | 1900 | N | ASN | A | 411 | 122.406 | 89.531 | 62.137 | 1.00 | 39.32 | 7 |
| ATOM | 1901 | CA | ASN | A | 411 | 122.266 | 90.219 | 60.902 | 1.00 | 41.98 | 6 |
| ATOM | 1902 | CB | ASN | A | 411 | 122.567 | 91.680 | 61.016 | 1.00 | 44.32 | 6 |
| ATOM | 1903 | CG | ASN | A | 411 | 121.555 | 92.308 | 61.895 | 1.00 | 47.64 | 6 |
| ATOM | 1904 | OD1 | ASN | A | 411 | 121.449 | 91.971 | 63.062 | 1.00 | 50.47 | 8 |
| ATOM | 1905 | ND2 | ASN | A | 411 | 120.793 | 93.240 | 61.354 | 1.00 | 48.74 | 7 |
| ATOM | 1906 | C | ASN | A | 411 | 123.221 | 89.584 | 59.975 | 1.00 | 40.86 | 6 |
| ATOM | 1907 | O | ASN | A | 411 | 124.329 | 90.031 | 59.685 | 1.00 | 41.14 | 8 |
| ATOM | 1908 | N | VAL | A | 412 | 122.698 | 88.477 | 59.540 | 1.00 | 40.98 | 7 |
| ATOM | 1909 | CA | VAL | A | 412 | 123.397 | 87.656 | 58.631 | 1.00 | 41.10 | 6 |
| ATOM | 1910 | CB | VAL | A | 412 | 124.250 | 86.610 | 59.359 | 1.00 | 43.78 | 6 |
| ATOM | 1911 | CG1 | VAL | A | 412 | 124.947 | 85.712 | 58.347 | 1.00 | 44.32 | 6 |
| ATOM | 1912 | CG2 | VAL | A | 412 | 125.270 | 87.264 | 60.278 | 1.00 | 46.04 | 6 |
| ATOM | 1913 | C | VAL | A | 412 | 122.410 | 86.925 | 57.738 | 1.00 | 39.81 | 6 |
| ATOM | 1914 | O | VAL | A | 412 | 121.390 | 86.386 | 58.179 | 1.00 | 39.00 | 8 |
| ATOM | 1915 | N | TRP | A | 413 | 122.750 | 86.925 | 56.485 | 1.00 | 37.64 | 7 |
| ATOM | 1916 | CA | TRP | A | 413 | 121.929 | 86.216 | 55.510 | 1.00 | 37.95 | 6 |
| ATOM | 1917 | CB | TRP | A | 413 | 122.215 | 86.692 | 54.083 | 1.00 | 36.04 | 6 |
| ATOM | 1918 | CG | TRP | A | 413 | 121.594 | 88.000 | 53.711 | 1.00 | 36.62 | 6 |
| ATOM | 1919 | CD2 | TRP | A | 413 | 120.210 | 88.247 | 53.444 | 1.00 | 35.86 | 6 |
| ATOM | 1920 | CE2 | TRP | A | 413 | 120.085 | 89.614 | 53.118 | 1.00 | 37.12 | 6 |
| ATOM | 1921 | CE3 | TRP | A | 413 | 119.064 | 87.445 | 53.449 | 1.00 | 36.57 | 6 |
| ATOM | 1922 | CD1 | TRP | A | 413 | 122.236 | 89.197 | 53.540 | 1.00 | 37.75 | 6 |
| ATOM | 1923 | NE1 | TRP | A | 413 | 121.336 | 90.170 | 53.183 | 1.00 | 36.62 | 7 |
| ATOM | 1924 | CZ2 | TRP | A | 413 | 118.858 | 90.199 | 52.799 | 1.00 | 37.73 | 6 |
| ATOM | 1925 | CZ3 | TRP | A | 413 | 117.842 | 88.026 | 53.132 | 1.00 | 38.34 | 6 |
| ATOM | 1926 | CH2 | TRP | A | 413 | 117.749 | 89.391 | 52.813 | 1.00 | 38.59 | 6 |
| ATOM | 1927 | C | TRP | A | 413 | 122.242 | 84.727 | 55.578 | 1.00 | 38.27 | 6 |
| ATOM | 1928 | O | TRP | A | 413 | 123.410 | 84.333 | 55.609 | 1.00 | 39.21 | 8 |
| ATOM | 1929 | N | LEU | A | 414 | 121.200 | 83.905 | 55.623 | 1.00 | 39.34 | 7 |
| ATOM | 1930 | CA | LEU | A | 414 | 121.364 | 82.455 | 55.627 | 1.00 | 39.63 | 6 |
| ATOM | 1931 | CB | LEU | A | 414 | 120.519 | 81.804 | 56.725 | 1.00 | 38.33 | 6 |
| ATOM | 1932 | CG | LEU | A | 414 | 120.941 | 82.040 | 58.177 | 1.00 | 39.08 | 6 |
| ATOM | 1933 | CD1 | LEU | A | 414 | 120.055 | 81.217 | 59.092 | 1.00 | 35.65 | 6 |
| ATOM | 1934 | CD2 | LEU | A | 414 | 122.414 | 81.654 | 58.372 | 1.00 | 36.45 | 6 |
| ATOM | 1935 | C | LEU | A | 414 | 120.840 | 82.046 | 54.259 | 1.00 | 41.16 | 6 |
| ATOM | 1936 | O | LEU | A | 414 | 119.647 | 82.180 | 53.989 | 1.00 | 41.85 | 8 |
| ATOM | 1937 | N | GLU | A | 415 | 121.729 | 81.577 | 53.387 | 1.00 | 42.52 | 7 |
| ATOM | 1938 | CA | GLU | A | 415 | 121.326 | 81.187 | 52.042 | 1.00 | 42.42 | 6 |
| ATOM | 1939 | CB | GLU | A | 415 | 122.293 | 81.767 | 51.014 | 1.00 | 44.14 | 6 |
| ATOM | 1940 | CG | GLU | A | 415 | 121.856 | 81.528 | 49.581 | 1.00 | 46.84 | 6 |
| ATOM | 1941 | CD | GLU | A | 415 | 122.688 | 82.307 | 48.583 | 1.00 | 49.89 | 6 |
| ATOM | 1942 | OE1 | GLU | A | 415 | 123.886 | 81.985 | 48.415 | 1.00 | 50.42 | 8 |
| ATOM | 1943 | OE2 | GLU | A | 415 | 122.141 | 83.251 | 47.973 | 1.00 | 50.86 | 8 |
| ATOM | 1944 | C | GLU | A | 415 | 121.224 | 79.682 | 51.854 | 1.00 | 43.07 | 6 |
| ATOM | 1945 | O | GLU | A | 415 | 122.127 | 78.930 | 52.225 | 1.00 | 43.39 | 8 |
| ATOM | 1946 | N | PHE | A | 416 | 120.114 | 79.224 | 51.263 | 1.00 | 43.16 | 7 |
| ATOM | 1947 | CA | PHE | A | 416 | 119.863 | 77.809 | 51.028 | 1.00 | 44.50 | 6 |
| ATOM | 1948 | CB | PHE | A | 416 | 118.385 | 77.497 | 51.397 | 1.00 | 42.87 | 6 |
| ATOM | 1949 | CG | PHE | A | 416 | 118.078 | 77.783 | 52.834 | 1.00 | 43.14 | 6 |
| ATOM | 1950 | CD1 | PHE | A | 416 | 117.940 | 79.094 | 53.283 | 1.00 | 42.19 | 6 |
| ATOM | 1951 | CD2 | PHE | A | 416 | 117.940 | 76.745 | 53.748 | 1.00 | 40.55 | 6 |
| ATOM | 1952 | CE1 | PHE | A | 416 | 117.673 | 79.363 | 54.626 | 1.00 | 41.92 | 6 |
| ATOM | 1953 | CE2 | PHE | A | 416 | 117.673 | 77.005 | 55.091 | 1.00 | 41.15 | 6 |
| ATOM | 1954 | CZ | PHE | A | 416 | 117.542 | 78.316 | 55.529 | 1.00 | 39.46 | 6 |
| ATOM | 1955 | C | PHE | A | 416 | 120.200 | 77.346 | 49.611 | 1.00 | 45.13 | 6 |
| ATOM | 1956 | O | PHE | A | 416 | 120.489 | 78.166 | 48.727 | 1.00 | 44.88 | 8 |
| ATOM | 1957 | N | SER | A | 417 | 120.159 | 76.047 | 49.412 | 1.00 | 45.55 | 7 |
| ATOM | 1958 | CA | SER | A | 417 | 120.439 | 75.427 | 48.121 | 1.00 | 46.85 | 6 |
| ATOM | 1959 | CB | SER | A | 417 | 120.841 | 73.954 | 48.302 | 1.00 | 47.92 | 6 |
| ATOM | 1960 | OG | SER | A | 417 | 119.843 | 73.250 | 49.019 | 1.00 | 52.74 | 8 |
| ATOM | 1961 | C | SER | A | 417 | 119.219 | 75.473 | 47.247 | 1.00 | 44.96 | 6 |
| ATOM | 1962 | O | SER | A | 417 | 119.262 | 75.377 | 46.027 | 1.00 | 46.34 | 8 |
| ATOM | 1963 | N | ILE | A | 418 | 118.145 | 75.640 | 47.947 | 1.00 | 42.56 | 7 |
| ATOM | 1964 | CA | ILE | A | 418 | 116.852 | 75.654 | 47.342 | 1.00 | 38.93 | 6 |
| ATOM | 1965 | CB | ILE | A | 418 | 115.817 | 75.434 | 48.425 | 1.00 | 39.56 | 6 |
| ATOM | 1966 | CG2 | ILE | A | 418 | 114.431 | 75.275 | 47.845 | 1.00 | 39.65 | 6 |
| ATOM | 1967 | CG1 | ILE | A | 418 | 116.204 | 74.194 | 49.219 | 1.00 | 42.20 | 6 |
| ATOM | 1968 | CD1 | ILE | A | 418 | 115.040 | 73.489 | 49.863 | 1.00 | 44.49 | 6 |
| ATOM | 1969 | C | ILE | A | 418 | 116.549 | 76.884 | 46.557 | 1.00 | 37.83 | 6 |
| ATOM | 1970 | O | ILE | A | 418 | 116.855 | 78.012 | 46.941 | 1.00 | 36.31 | 8 |
| ATOM | 1971 | N | LYS | A | 419 | 115.940 | 76.598 | 45.435 | 1.00 | 34.70 | 7 |
| ATOM | 1972 | CA | LYS | A | 419 | 115.472 | 77.622 | 44.574 | 1.00 | 34.79 | 6 |
| ATOM | 1973 | CB | LYS | A | 419 | 115.507 | 77.200 | 43.106 | 1.00 | 33.82 | 6 |
| ATOM | 1974 | CG | LYS | A | 419 | 116.892 | 76.809 | 42.612 | 1.00 | 37.02 | 6 |
| ATOM | 1975 | CD | LYS | A | 419 | 116.914 | 76.613 | 41.108 | 1.00 | 37.07 | 6 |
| ATOM | 1976 | CE | LYS | A | 419 | 118.339 | 76.555 | 40.577 | 1.00 | 38.11 | 6 |
| ATOM | 1977 | NZ | LYS | A | 419 | 118.392 | 76.669 | 39.093 | 1.00 | 41.00 | 7 |
| ATOM | 1978 | C | LYS | A | 419 | 114.051 | 77.975 | 44.984 | 1.00 | 35.62 | 6 |
| ATOM | 1979 | O | LYS | A | 419 | 113.258 | 77.100 | 45.361 | 1.00 | 34.14 | 8 |
| ATOM | 1980 | N | ILE | A | 420 | 113.734 | 79.259 | 44.886 | 1.00 | 35.91 | 7 |
| ATOM | 1981 | CA | ILE | A | 420 | 112.412 | 79.761 | 45.236 | 1.00 | 36.91 | 6 |
| ATOM | 1982 | CB | ILE | A | 420 | 112.279 | 81.262 | 44.884 | 1.00 | 35.83 | 6 |
| ATOM | 1983 | CG2 | ILE | A | 420 | 110.873 | 81.744 | 45.187 | 1.00 | 35.04 | 6 |
| ATOM | 1984 | CG1 | ILE | A | 420 | 113.327 | 82.071 | 45.644 | 1.00 | 38.85 | 6 |
| ATOM | 1985 | CD1 | ILE | A | 420 | 113.192 | 82.005 | 47.153 | 1.00 | 40.77 | 6 |
| ATOM | 1986 | C | ILE | A | 420 | 111.334 | 78.991 | 44.483 | 1.00 | 36.51 | 6 |
| ATOM | 1987 | O | ILE | A | 420 | 110.290 | 78.647 | 45.040 | 1.00 | 37.31 | 8 |
| ATOM | 1988 | N | LYS | A | 421 | 111.596 | 78.734 | 43.209 | 1.00 | 36.28 | 7 |
| ATOM | 1989 | CA | LYS | A | 421 | 110.661 | 78.017 | 42.359 | 1.00 | 37.18 | 6 |
| ATOM | 1990 | CB | LYS | A | 421 | 111.237 | 77.916 | 40.942 | 1.00 | 39.99 | 6 |
| ATOM | 1991 | CG | LYS | A | 421 | 110.267 | 77.414 | 39.891 | 1.00 | 43.60 | 6 |
| ATOM | 1992 | CD | LYS | A | 421 | 109.189 | 78.458 | 39.576 | 1.00 | 47.47 | 6 |
| ATOM | 1993 | CE | LYS | A | 421 | 108.334 | 78.045 | 38.372 | 1.00 | 47.74 | 6 |
| ATOM | 1994 | NZ | LYS | A | 421 | 107.260 | 79.046 | 38.091 | 1.00 | 50.10 | 7 |
| ATOM | 1995 | C | LYS | A | 421 | 110.399 | 76.614 | 42.903 | 1.00 | 35.84 | 6 |
| ATOM | 1996 | O | LYS | A | 421 | 109.345 | 76.028 | 42.653 | 1.00 | 37.27 | 8 |
| ATOM | 1997 | N | ASP | A | 422 | 111.364 | 76.080 | 43.647 | 1.00 | 34.27 | 7 |
| ATOM | 1998 | CA | ASP | A | 422 | 111.254 | 74.734 | 44.203 | 1.00 | 32.80 | 6 |
| ATOM | 1999 | CB | ASP | A | 422 | 112.644 | 74.101 | 44.295 | 1.00 | 32.04 | 6 |
| ATOM | 2000 | CG | ASP | A | 422 | 113.176 | 73.670 | 42.936 | 1.00 | 34.49 | 6 |
| ATOM | 2001 | OD1 | ASP | A | 422 | 112.395 | 73.055 | 42.180 | 1.00 | 34.95 | 8 |
| ATOM | 2002 | OD2 | ASP | A | 422 | 114.363 | 73.931 | 42.628 | 1.00 | 33.45 | 8 |
| ATOM | 2003 | C | ASP | A | 422 | 110.537 | 74.608 | 45.550 | 1.00 | 31.18 | 6 |
| ATOM | 2004 | O | ASP | A | 422 | 110.361 | 73.496 | 46.059 | 1.00 | 28.58 | 8 |
| ATOM | 2005 | N | LEU | A | 423 | 110.117 | 75.734 | 46.115 | 1.00 | 30.39 | 7 |
| ATOM | 2006 | CA | LEU | A | 423 | 109.411 | 75.720 | 47.393 | 1.00 | 29.32 | 6 |
| ATOM | 2007 | CB | LEU | A | 423 | 109.322 | 77.135 | 47.978 | 1.00 | 29.35 | 6 |
| ATOM | 2008 | CG | LEU | A | 423 | 110.651 | 77.738 | 48.446 | 1.00 | 32.26 | 6 |
| ATOM | 2009 | CD1 | LEU | A | 423 | 110.424 | 79.150 | 48.985 | 1.00 | 30.48 | 6 |
| ATOM | 2010 | CD2 | LEU | A | 423 | 111.261 | 76.859 | 49.530 | 1.00 | 31.11 | 6 |
| ATOM | 2011 | C | LEU | A | 423 | 108.016 | 75.134 | 47.221 | 1.00 | 28.48 | 6 |
| ATOM | 2012 | O | LEU | A | 423 | 107.171 | 75.698 | 46.527 | 1.00 | 31.21 | 8 |
| ATOM | 2013 | N | PRO | A | 424 | 107.760 | 73.976 | 47.849 | 1.00 | 28.80 | 7 |
| ATOM | 2014 | CD | PRO | A | 424 | 108.689 | 73.196 | 48.691 | 1.00 | 27.10 | 6 |
| ATOM | 2015 | CA | PRO | A | 424 | 106.453 | 73.318 | 47.755 | 1.00 | 28.99 | 6 |
| ATOM | 2016 | CB | PRO | A | 424 | 106.755 | 71.900 | 48.215 | 1.00 | 28.81 | 6 |
| ATOM | 2017 | CG | PRO | A | 424 | 107.773 | 72.135 | 49.312 | 1.00 | 29.32 | 6 |
| ATOM | 2018 | C | PRO | A | 424 | 105.424 | 74.002 | 48.648 | 1.00 | 28.92 | 6 |
| ATOM | 2019 | O | PRO | A | 424 | 105.746 | 74.427 | 49.754 | 1.00 | 28.30 | 8 |
| ATOM | 2020 | N | LYS | A | 425 | 104.190 | 74.117 | 48.174 | 1.00 | 29.87 | 7 |
| ATOM | 2021 | CA | LYS | A | 425 | 103.161 | 74.738 | 48.986 | 1.00 | 32.47 | 6 |
| ATOM | 2022 | CB | LYS | A | 425 | 101.801 | 74.709 | 48.287 | 1.00 | 34.55 | 6 |
| ATOM | 2023 | CG | LYS | A | 425 | 100.707 | 75.409 | 49.104 | 1.00 | 38.26 | 6 |
| ATOM | 2024 | CD | LYS | A | 425 | 99.312 | 75.289 | 48.500 | 1.00 | 39.70 | 6 |
| ATOM | 2025 | CE | LYS | A | 425 | 98.309 | 76.052 | 49.369 | 1.00 | 41.40 | 6 |
| ATOM | 2026 | NZ | LYS | A | 425 | 96.896 | 75.917 | 48.915 | 1.00 | 40.74 | 7 |
| ATOM | 2027 | C | LYS | A | 425 | 103.091 | 73.924 | 50.277 | 1.00 | 32.44 | 6 |
| ATOM | 2028 | O | LYS | A | 425 | 103.188 | 72.696 | 50.250 | 1.00 | 31.50 | 8 |
| ATOM | 2029 | N | GLY | A | 426 | 102.940 | 74.614 | 51.400 | 1.00 | 31.94 | 7 |
| ATOM | 2030 | CA | GLY | A | 426 | 102.865 | 73.935 | 52.682 | 1.00 | 30.15 | 6 |
| ATOM | 2031 | C | GLY | A | 426 | 104.210 | 73.889 | 53.373 | 1.00 | 30.43 | 6 |
| ATOM | 2032 | O | GLY | A | 426 | 104.334 | 73.399 | 54.501 | 1.00 | 30.37 | 8 |
| ATOM | 2033 | N | ALA | A | 427 | 105.228 | 74.398 | 52.687 | 1.00 | 28.41 | 7 |
| ATOM | 2034 | CA | ALA | A | 427 | 106.571 | 74.424 | 53.237 | 1.00 | 28.07 | 6 |
| ATOM | 2035 | CB | ALA | A | 427 | 107.561 | 74.916 | 52.182 | 1.00 | 29.08 | 6 |
| ATOM | 2036 | C | ALA | A | 427 | 106.613 | 75.348 | 54.441 | 1.00 | 26.40 | 6 |
| ATOM | 2037 | O | ALA | A | 427 | 105.970 | 76.399 | 54.437 | 1.00 | 23.37 | 8 |
| ATOM | 2038 | N | LEU | A | 428 | 107.369 | 74.948 | 55.463 | 1.00 | 24.40 | 7 |
| ATOM | 2039 | CA | LEU | A | 428 | 107.537 | 75.744 | 56.676 | 1.00 | 24.90 | 6 |
| ATOM | 2040 | CB | LEU | A | 428 | 107.180 | 74.940 | 57.926 | 1.00 | 24.99 | 6 |
| ATOM | 2041 | CG | LEU | A | 428 | 105.739 | 74.646 | 58.315 | 1.00 | 24.59 | 6 |
| ATOM | 2042 | CD1 | LEU | A | 428 | 105.726 | 73.872 | 59.631 | 1.00 | 25.87 | 6 |
| ATOM | 2043 | CD2 | LEU | A | 428 | 104.989 | 75.960 | 58.439 | 1.00 | 24.90 | 6 |
| ATOM | 2044 | C | LEU | A | 428 | 108.975 | 76.190 | 56.875 | 1.00 | 26.32 | 6 |
| ATOM | 2045 | O | LEU | A | 428 | 109.908 | 75.418 | 56.623 | 1.00 | 27.28 | 8 |
| ATOM | 2046 | N | LEU | A | 429 | 109.160 | 77.433 | 57.307 | 1.00 | 23.04 | 7 |
| ATOM | 2047 | CA | LEU | A | 429 | 110.494 | 77.884 | 57.653 | 1.00 | 24.19 | 6 |
| ATOM | 2048 | CB | LEU | A | 429 | 110.719 | 79.370 | 57.356 | 1.00 | 23.52 | 6 |
| ATOM | 2049 | CG | LEU | A | 429 | 112.028 | 79.888 | 57.983 | 1.00 | 23.76 | 6 |
| ATOM | 2050 | CD1 | LEU | A | 429 | 113.212 | 79.151 | 57.357 | 1.00 | 22.16 | 6 |
| ATOM | 2051 | CD2 | LEU | A | 429 | 112.188 | 81.399 | 57.785 | 1.00 | 22.26 | 6 |
| ATOM | 2052 | C | LEU | A | 429 | 110.492 | 77.679 | 59.172 | 1.00 | 24.77 | 6 |
| ATOM | 2053 | O | LEU | A | 429 | 109.832 | 78.426 | 59.903 | 1.00 | 24.51 | 8 |
| ATOM | 2054 | N | ASN | A | 430 | 111.196 | 76.657 | 59.639 | 1.00 | 24.85 | 7 |
| ATOM | 2055 | CA | ASN | A | 430 | 111.279 | 76.356 | 61.068 | 1.00 | 25.15 | 6 |
| ATOM | 2056 | CB | ASN | A | 430 | 111.329 | 74.828 | 61.252 | 1.00 | 26.25 | 6 |
| ATOM | 2057 | CG | ASN | A | 430 | 111.692 | 74.404 | 62.659 | 1.00 | 27.16 | 6 |
| ATOM | 2058 | OD1 | ASN | A | 430 | 112.850 | 74.082 | 62.939 | 1.00 | 29.96 | 8 |
| ATOM | 2059 | ND2 | ASN | A | 430 | 110.704 | 74.389 | 63.557 | 1.00 | 25.59 | 7 |
| ATOM | 2060 | C | ASN | A | 430 | 112.525 | 77.037 | 61.651 | 1.00 | 26.13 | 6 |
| ATOM | 2061 | O | ASN | A | 430 | 113.633 | 76.857 | 61.142 | 1.00 | 26.42 | 8 |
| ATOM | 2062 | N | LEU | A | 431 | 112.341 | 77.833 | 62.703 | 1.00 | 22.85 | 7 |
| ATOM | 2063 | CA | LEU | A | 431 | 113.455 | 78.555 | 63.317 | 1.00 | 22.51 | 6 |
| ATOM | 2064 | CB | LEU | A | 431 | 113.202 | 80.062 | 63.267 | 1.00 | 22.56 | 6 |
| ATOM | 2065 | CG | LEU | A | 431 | 113.018 | 80.678 | 61.872 | 1.00 | 23.30 | 6 |
| ATOM | 2066 | CD1 | LEU | A | 431 | 112.410 | 82.071 | 62.025 | 1.00 | 22.19 | 6 |
| ATOM | 2067 | CD2 | LEU | A | 431 | 114.367 | 80.760 | 61.127 | 1.00 | 22.68 | 6 |
| ATOM | 2068 | C | LEU | A | 431 | 113.625 | 78.113 | 64.759 | 1.00 | 23.68 | 6 |
| ATOM | 2069 | O | LEU | A | 431 | 112.684 | 78.151 | 65.548 | 1.00 | 22.30 | 8 |
| ATOM | 2070 | N | GLN | A | 432 | 114.836 | 77.710 | 65.113 | 1.00 | 25.65 | 7 |
| ATOM | 2071 | CA | GLN | A | 432 | 115.068 | 77.238 | 66.464 | 1.00 | 24.30 | 6 |
| ATOM | 2072 | CB | GLN | A | 432 | 115.110 | 75.701 | 66.459 | 1.00 | 25.96 | 6 |
| ATOM | 2073 | CG | GLN | A | 432 | 113.728 | 75.109 | 66.179 | 1.00 | 26.25 | 6 |
| ATOM | 2074 | CD | GLN | A | 432 | 113.699 | 73.600 | 66.053 | 1.00 | 26.28 | 6 |
| ATOM | 2075 | OE1 | GLN | A | 432 | 112.644 | 72.999 | 66.179 | 1.00 | 29.03 | 8 |
| ATOM | 2076 | NE2 | GLN | A | 432 | 114.844 | 72.987 | 65.781 | 1.00 | 29.91 | 7 |
| ATOM | 2077 | C | GLN | A | 432 | 116.299 | 77.826 | 67.124 | 1.00 | 25.76 | 6 |
| ATOM | 2078 | O | GLN | A | 432 | 117.271 | 78.195 | 66.463 | 1.00 | 25.83 | 8 |
| ATOM | 2079 | N | ILE | A | 433 | 116.234 | 77.930 | 68.445 | 1.00 | 25.41 | 7 |
| ATOM | 2080 | CA | ILE | A | 433 | 117.331 | 78.483 | 69.209 | 1.00 | 29.08 | 6 |
| ATOM | 2081 | CB | ILE | A | 433 | 116.862 | 79.708 | 70.016 | 1.00 | 30.56 | 6 |
| ATOM | 2082 | CG2 | ILE | A | 433 | 118.024 | 80.309 | 70.785 | 1.00 | 32.44 | 6 |
| ATOM | 2083 | CG1 | ILE | A | 433 | 116.280 | 80.757 | 69.058 | 1.00 | 30.98 | 6 |
| ATOM | 2084 | CD1 | ILE | A | 433 | 115.875 | 82.044 | 69.744 | 1.00 | 35.38 | 6 |
| ATOM | 2085 | C | ILE | A | 433 | 117.884 | 77.408 | 70.134 | 1.00 | 30.49 | 6 |
| ATOM | 2086 | O | ILE | A | 433 | 117.152 | 76.820 | 70.937 | 1.00 | 30.63 | 8 |
| ATOM | 2087 | N | TYR | A | 434 | 119.176 | 77.141 | 69.983 | 1.00 | 33.94 | 7 |
| ATOM | 2088 | CA | TYR | A | 434 | 119.875 | 76.141 | 70.781 | 1.00 | 35.52 | 6 |
| ATOM | 2089 | CB | TYR | A | 434 | 120.702 | 75.229 | 69.866 | 1.00 | 34.69 | 6 |
| ATOM | 2090 | CG | TYR | A | 434 | 119.869 | 74.277 | 69.037 | 1.00 | 33.73 | 6 |
| ATOM | 2091 | CD1 | TYR | A | 434 | 119.451 | 73.053 | 69.552 | 1.00 | 33.37 | 6 |
| ATOM | 2092 | CE1 | TYR | A | 434 | 118.662 | 72.178 | 68.792 | 1.00 | 34.14 | 6 |
| ATOM | 2093 | CD2 | TYR | A | 434 | 119.478 | 74.612 | 67.739 | 1.00 | 33.83 | 6 |
| ATOM | 2094 | CE2 | TYR | A | 434 | 118.694 | 73.753 | 66.978 | 1.00 | 33.17 | 6 |
| ATOM | 2095 | CZ | TYR | A | 434 | 118.290 | 72.543 | 67.505 | 1.00 | 33.50 | 6 |
| ATOM | 2096 | OH | TYR | A | 434 | 117.507 | 71.707 | 66.751 | 1.00 | 36.20 | 8 |
| ATOM | 2097 | C | TYR | A | 434 | 120.789 | 76.825 | 71.792 | 1.00 | 37.82 | 6 |
| ATOM | 2098 | O | TYR | A | 434 | 121.382 | 77.856 | 71.498 | 1.00 | 36.48 | 8 |
| ATOM | 2099 | N | CYS | A | 435 | 120.889 | 76.240 | 72.982 | 1.00 | 40.03 | 7 |
| ATOM | 2100 | CA | CYS | A | 435 | 121.718 | 76.789 | 74.049 | 1.00 | 45.88 | 6 |
| ATOM | 2101 | CB | CYS | A | 435 | 120.831 | 77.289 | 75.192 | 1.00 | 48.29 | 6 |
| ATOM | 2102 | SG | CYS | A | 435 | 121.716 | 78.102 | 76.547 | 1.00 | 57.50 | 16 |
| ATOM | 2103 | C | CYS | A | 435 | 122.673 | 75.728 | 74.577 | 1.00 | 45.55 | 6 |
| ATOM | 2104 | O | CYS | A | 435 | 122.280 | 74.573 | 74.749 | 1.00 | 48.00 | 8 |
| ATOM | 2105 | N | LYS | A | 457 | 126.651 | 69.079 | 74.729 | 1.00 | 20.00 | 7 |
| ATOM | 2106 | CA | LYS | A | 457 | 125.487 | 69.039 | 73.861 | 1.00 | 20.00 | 6 |
| ATOM | 2107 | C | LYS | A | 457 | 125.386 | 70.312 | 73.034 | 1.00 | 20.00 | 6 |
| ATOM | 2108 | O | LYS | A | 457 | 126.215 | 71.250 | 73.319 | 1.00 | 20.00 | 8 |
| ATOM | 2109 | CB | LYS | A | 457 | 124.222 | 68.813 | 74.687 | 1.00 | 20.00 | 6 |
| ATOM | 2110 | CG | LYS | A | 457 | 124.219 | 67.503 | 75.459 | 1.00 | 20.00 | 6 |
| ATOM | 2111 | CD | LYS | A | 457 | 122.859 | 67.228 | 76.075 | 1.00 | 20.00 | 6 |
| ATOM | 2112 | CE | LYS | A | 457 | 122.860 | 65.921 | 76.853 | 1.00 | 20.00 | 6 |
| ATOM | 2113 | NZ | LYS | A | 457 | 121.532 | 65.633 | 77.459 | 1.00 | 20.00 | 7 |
| ATOM | 2114 | N | VAL | A | 458 | 124.220 | 70.143 | 72.413 | 1.00 | 20.00 | 7 |
| ATOM | 2115 | CA | VAL | A | 458 | 123.249 | 70.806 | 71.584 | 1.00 | 20.00 | 6 |
| ATOM | 2116 | C | VAL | A | 458 | 121.986 | 71.019 | 72.477 | 1.00 | 20.00 | 6 |
| ATOM | 2117 | O | VAL | A | 458 | 121.635 | 70.115 | 73.222 | 1.00 | 20.00 | 8 |
| ATOM | 2118 | CB | VAL | A | 458 | 122.794 | 69.850 | 70.442 | 1.00 | 20.00 | 6 |
| ATOM | 2119 | CG1 | VAL | A | 458 | 122.530 | 70.626 | 69.168 | 1.00 | 20.00 | 6 |
| ATOM | 2120 | CG2 | VAL | A | 458 | 123.835 | 68.764 | 70.201 | 1.00 | 20.00 | 6 |
| ATOM | 2121 | N | ARG | A | 459 | 121.303 | 72.150 | 72.428 | 1.00 | 20.00 | 7 |
| ATOM | 2122 | CA | ARG | A | 459 | 120.049 | 72.190 | 73.191 | 1.00 | 20.00 | 6 |
| ATOM | 2123 | C | ARG | A | 459 | 119.091 | 73.224 | 72.627 | 1.00 | 20.00 | 6 |
| ATOM | 2124 | O | ARG | A | 459 | 119.417 | 74.382 | 72.431 | 1.00 | 20.00 | 8 |
| ATOM | 2125 | CB | ARG | A | 459 | 120.297 | 72.310 | 74.702 | 1.00 | 20.00 | 6 |
| ATOM | 2126 | CG | ARG | A | 459 | 120.752 | 71.006 | 75.381 | 1.00 | 20.00 | 6 |
| ATOM | 2127 | CD | ARG | A | 459 | 119.953 | 70.557 | 76.630 | 1.00 | 20.00 | 6 |
| ATOM | 2128 | NE | ARG | A | 459 | 120.660 | 69.508 | 77.397 | 1.00 | 20.00 | 7 |
| ATOM | 2129 | CZ | ARG | A | 459 | 120.307 | 69.029 | 78.593 | 1.00 | 20.00 | 6 |
| ATOM | 2130 | NH1 | ARG | A | 459 | 119.227 | 69.478 | 79.218 | 1.00 | 20.00 | 7 |
| ATOM | 2131 | NH2 | ARG | A | 459 | 121.065 | 68.101 | 79.162 | 1.00 | 20.00 | 7 |
| ATOM | 2132 | N | LEU | A | 460 | 117.909 | 72.731 | 72.364 | 1.00 | 43.17 | 7 |
| ATOM | 2133 | CA | LEU | A | 460 | 116.809 | 73.500 | 71.819 | 1.00 | 41.32 | 6 |
| ATOM | 2134 | CB | LEU | A | 460 | 115.884 | 72.595 | 70.998 | 1.00 | 40.23 | 6 |
| ATOM | 2135 | CG | LEU | A | 460 | 114.649 | 73.239 | 70.353 | 1.00 | 39.58 | 6 |
| ATOM | 2136 | CD1 | LEU | A | 460 | 115.042 | 74.283 | 69.308 | 1.00 | 35.51 | 6 |
| ATOM | 2137 | CD2 | LEU | A | 460 | 113.778 | 72.160 | 69.727 | 1.00 | 37.87 | 6 |
| ATOM | 2138 | C | LEU | A | 460 | 116.014 | 74.149 | 72.955 | 1.00 | 40.53 | 6 |
| ATOM | 2139 | O | LEU | A | 460 | 115.479 | 73.457 | 73.825 | 1.00 | 41.80 | 8 |
| ATOM | 2140 | N | LEU | A | 461 | 115.939 | 75.476 | 72.937 | 1.00 | 38.69 | 7 |
| ATOM | 2141 | CA | LEU | A | 461 | 115.210 | 76.225 | 73.958 | 1.00 | 35.79 | 6 |
| ATOM | 2142 | CB | LEU | A | 461 | 116.097 | 77.323 | 74.554 | 1.00 | 38.17 | 6 |
| ATOM | 2143 | CG | LEU | A | 461 | 117.311 | 76.920 | 75.389 | 1.00 | 40.60 | 6 |
| ATOM | 2144 | CD1 | LEU | A | 461 | 117.865 | 78.161 | 76.076 | 1.00 | 41.76 | 6 |
| ATOM | 2145 | CD2 | LEU | A | 461 | 116.907 | 75.876 | 76.425 | 1.00 | 42.07 | 6 |
| ATOM | 2146 | C | LEU | A | 461 | 113.932 | 76.877 | 73.432 | 1.00 | 32.67 | 6 |
| ATOM | 2147 | O | LEU | A | 461 | 112.959 | 77.031 | 74.168 | 1.00 | 29.28 | 8 |
| ATOM | 2148 | N | TYR | A | 462 | 113.940 | 77.267 | 72.162 | 1.00 | 29.97 | 7 |
| ATOM | 2149 | CA | TYR | A | 462 | 112.782 | 77.933 | 71.572 | 1.00 | 27.52 | 6 |
| ATOM | 2150 | CB | TYR | A | 462 | 112.957 | 79.455 | 71.607 | 1.00 | 24.52 | 6 |
| ATOM | 2151 | CG | TYR | A | 462 | 113.228 | 80.040 | 72.974 | 1.00 | 27.06 | 6 |
| ATOM | 2152 | CD1 | TYR | A | 462 | 112.188 | 80.377 | 73.837 | 1.00 | 26.78 | 6 |
| ATOM | 2153 | CE1 | TYR | A | 462 | 112.453 | 80.889 | 75.116 | 1.00 | 27.50 | 6 |
| ATOM | 2154 | CD2 | TYR | A | 462 | 114.535 | 80.229 | 73.415 | 1.00 | 27.33 | 6 |
| ATOM | 2155 | CE2 | TYR | A | 462 | 114.807 | 80.731 | 74.678 | 1.00 | 27.86 | 6 |
| ATOM | 2156 | CZ | TYR | A | 462 | 113.771 | 81.059 | 75.526 | 1.00 | 28.84 | 6 |
| ATOM | 2157 | OH | TYR | A | 462 | 114.073 | 81.541 | 76.793 | 1.00 | 31.44 | 8 |
| ATOM | 2158 | C | TYR | A | 462 | 112.618 | 77.529 | 70.118 | 1.00 | 26.40 | 6 |
| ATOM | 2159 | O | TYR | A | 462 | 113.588 | 77.164 | 69.451 | 1.00 | 26.10 | 8 |
| ATOM | 2160 | N | TYR | A | 463 | 111.388 | 77.635 | 69.632 | 1.00 | 23.46 | 7 |
| ATOM | 2161 | CA | TYR | A | 463 | 111.085 | 77.328 | 68.244 | 1.00 | 22.49 | 6 |
| ATOM | 2162 | CB | TYR | A | 463 | 110.766 | 75.834 | 68.074 | 1.00 | 21.60 | 6 |
| ATOM | 2163 | CG | TYR | A | 463 | 109.440 | 75.405 | 68.657 | 1.00 | 23.47 | 6 |
| ATOM | 2164 | CD1 | TYR | A | 463 | 108.265 | 75.462 | 67.894 | 1.00 | 22.63 | 6 |
| ATOM | 2165 | CE1 | TYR | A | 463 | 107.040 | 75.073 | 68.431 | 1.00 | 21.14 | 6 |
| ATOM | 2166 | CD2 | TYR | A | 463 | 109.347 | 74.950 | 69.971 | 1.00 | 22.36 | 6 |
| ATOM | 2167 | CE2 | TYR | A | 463 | 108.124 | 74.562 | 70.515 | 1.00 | 22.49 | 6 |
| ATOM | 2168 | CZ | TYR | A | 463 | 106.978 | 74.630 | 69.735 | 1.00 | 21.04 | 6 |
| ATOM | 2169 | OH | TYR | A | 463 | 105.778 | 74.261 | 70.275 | 1.00 | 23.91 | 8 |
| ATOM | 2170 | C | TYR | A | 463 | 109.888 | 78.146 | 67.807 | 1.00 | 20.40 | 6 |
| ATOM | 2171 | O | TYR | A | 463 | 109.080 | 78.595 | 68.627 | 1.00 | 21.37 | 8 |
| ATOM | 2172 | N | VAL | A | 464 | 109.788 | 78.357 | 66.508 | 1.00 | 19.74 | 7 |
| ATOM | 2173 | CA | VAL | A | 464 | 108.653 | 79.062 | 65.944 | 1.00 | 19.17 | 6 |
| ATOM | 2174 | CB | VAL | A | 464 | 108.752 | 80.591 | 66.120 | 1.00 | 18.10 | 6 |
| ATOM | 2175 | CG1 | VAL | A | 464 | 109.804 | 81.169 | 65.177 | 1.00 | 18.04 | 6 |
| ATOM | 2176 | CG2 | VAL | A | 464 | 107.385 | 81.226 | 65.837 | 1.00 | 18.80 | 6 |
| ATOM | 2177 | C | VAL | A | 464 | 108.696 | 78.717 | 64.468 | 1.00 | 19.31 | 6 |
| ATOM | 2178 | O | VAL | A | 464 | 109.772 | 78.478 | 63.921 | 1.00 | 20.14 | 8 |
| ATOM | 2179 | N | ASN | A | 465 | 107.534 | 78.681 | 63.831 | 1.00 | 19.75 | 7 |
| ATOM | 2180 | CA | ASN | A | 465 | 107.454 | 78.349 | 62.415 | 1.00 | 20.03 | 6 |
| ATOM | 2181 | CB | ASN | A | 465 | 106.680 | 77.052 | 62.224 | 1.00 | 21.26 | 6 |
| ATOM | 2182 | CG | ASN | A | 465 | 107.296 | 75.891 | 62.965 | 1.00 | 23.37 | 6 |
| ATOM | 2183 | OD1 | ASN | A | 465 | 108.472 | 75.564 | 62.773 | 1.00 | 22.27 | 8 |
| ATOM | 2184 | ND2 | ASN | A | 465 | 106.496 | 75.245 | 63.816 | 1.00 | 24.07 | 7 |
| ATOM | 2185 | C | ASN | A | 465 | 106.732 | 79.435 | 61.647 | 1.00 | 22.95 | 6 |
| ATOM | 2186 | O | ASN | A | 465 | 105.954 | 80.186 | 62.217 | 1.00 | 24.87 | 8 |
| ATOM | 2187 | N | LEU | A | 466 | 106.976 | 79.489 | 60.343 | 1.00 | 24.46 | 7 |
| ATOM | 2188 | CA | LEU | A | 466 | 106.332 | 80.461 | 59.455 | 1.00 | 25.68 | 6 |
| ATOM | 2189 | CB | LEU | A | 466 | 107.234 | 81.687 | 59.256 | 1.00 | 25.27 | 6 |
| ATOM | 2190 | CG | LEU | A | 466 | 106.817 | 82.721 | 58.207 | 1.00 | 29.13 | 6 |
| ATOM | 2191 | CD1 | LEU | A | 466 | 105.563 | 83.468 | 58.670 | 1.00 | 27.35 | 6 |
| ATOM | 2192 | CD2 | LEU | A | 466 | 107.967 | 83.696 | 57.977 | 1.00 | 27.46 | 6 |
| ATOM | 2193 | C | LEU | A | 466 | 106.105 | 79.780 | 58.114 | 1.00 | 23.55 | 6 |
| ATOM | 2194 | O | LEU | A | 466 | 107.044 | 79.239 | 57.534 | 1.00 | 23.34 | 8 |
| ATOM | 2195 | N | LEU | A | 467 | 104.867 | 79.786 | 57.633 | 1.00 | 24.66 | 7 |
| ATOM | 2196 | CA | LEU | A | 467 | 104.561 | 79.176 | 56.342 | 1.00 | 26.32 | 6 |
| ATOM | 2197 | CB | LEU | A | 467 | 103.044 | 79.143 | 56.120 | 1.00 | 27.18 | 6 |
| ATOM | 2198 | CG | LEU | A | 467 | 102.233 | 78.007 | 56.766 | 1.00 | 26.69 | 6 |
| ATOM | 2199 | CD1 | LEU | A | 467 | 100.785 | 78.455 | 56.932 | 1.00 | 24.55 | 6 |
| ATOM | 2200 | CD2 | LEU | A | 467 | 102.314 | 76.746 | 55.907 | 1.00 | 24.63 | 6 |
| ATOM | 2201 | C | LEU | A | 467 | 105.238 | 80.027 | 55.269 | 1.00 | 29.39 | 6 |
| ATOM | 2202 | O | LEU | A | 467 | 105.011 | 81.239 | 55.200 | 1.00 | 28.78 | 8 |
| ATOM | 2203 | N | LEU | A | 468 | 106.078 | 79.402 | 54.448 | 1.00 | 28.07 | 7 |
| ATOM | 2204 | CA | LEU | A | 468 | 106.786 | 80.128 | 53.404 | 1.00 | 29.90 | 6 |
| ATOM | 2205 | CB | LEU | A | 468 | 107.924 | 79.275 | 52.841 | 1.00 | 30.56 | 6 |
| ATOM | 2206 | CG | LEU | A | 468 | 109.175 | 79.247 | 53.717 | 1.00 | 29.84 | 6 |
| ATOM | 2207 | CD1 | LEU | A | 468 | 110.238 | 78.397 | 53.057 | 1.00 | 33.07 | 6 |
| ATOM | 2208 | CD2 | LEU | A | 468 | 109.694 | 80.667 | 53.918 | 1.00 | 30.28 | 6 |
| ATOM | 2209 | C | LEU | A | 468 | 105.860 | 80.586 | 52.285 | 1.00 | 29.24 | 6 |
| ATOM | 2210 | O | LEU | A | 468 | 106.065 | 81.649 | 51.704 | 1.00 | 32.99 | 8 |
| ATOM | 2211 | N | ILE | A | 469 | 104.875 | 79.754 | 52.002 | 1.00 | 26.73 | 7 |
| ATOM | 2212 | CA | ILE | A | 469 | 103.921 | 80.044 | 50.979 | 1.00 | 28.57 | 6 |
| ATOM | 2213 | CB | ILE | A | 469 | 103.821 | 78.926 | 49.929 | 1.00 | 29.79 | 6 |
| ATOM | 2214 | CG2 | ILE | A | 469 | 102.662 | 79.169 | 48.968 | 1.00 | 29.69 | 6 |
| ATOM | 2215 | CG1 | ILE | A | 469 | 105.134 | 78.876 | 49.169 | 1.00 | 32.72 | 6 |
| ATOM | 2216 | CD1 | ILE | A | 469 | 105.223 | 77.746 | 48.165 | 1.00 | 35.64 | 6 |
| ATOM | 2217 | C | ILE | A | 469 | 102.610 | 80.218 | 51.675 | 1.00 | 28.28 | 6 |
| ATOM | 2218 | O | ILE | A | 469 | 102.192 | 79.372 | 52.451 | 1.00 | 25.93 | 8 |
| ATOM | 2219 | N | ASP | A | 470 | 101.958 | 81.319 | 51.412 | 1.00 | 28.05 | 7 |
| ATOM | 2220 | CA | ASP | A | 470 | 100.708 | 81.451 | 52.064 | 1.00 | 31.36 | 6 |
| ATOM | 2221 | CB | ASP | A | 470 | 100.231 | 82.899 | 52.096 | 1.00 | 31.62 | 6 |
| ATOM | 2222 | CG | ASP | A | 470 | 100.141 | 83.509 | 50.741 | 1.00 | 32.80 | 6 |
| ATOM | 2223 | OD1 | ASP | A | 470 | 100.045 | 82.747 | 49.751 | 1.00 | 29.25 | 8 |
| ATOM | 2224 | OD2 | ASP | A | 470 | 100.149 | 84.753 | 50.642 | 1.00 | 35.14 | 8 |
| ATOM | 2225 | C | ASP | A | 470 | 99.703 | 80.542 | 51.434 | 1.00 | 32.37 | 6 |
| ATOM | 2226 | O | ASP | A | 470 | 100.026 | 79.525 | 50.819 | 1.00 | 30.74 | 8 |
| ATOM | 2227 | N | HIS | A | 471 | 98.461 | 80.911 | 51.619 | 1.00 | 32.41 | 7 |
| ATOM | 2228 | CA | HIS | A | 471 | 97.329 | 80.129 | 51.186 | 1.00 | 33.88 | 6 |
| ATOM | 2229 | CB | HIS | A | 471 | 96.165 | 80.415 | 52.106 | 1.00 | 35.21 | 6 |
| ATOM | 2230 | CG | HIS | A | 471 | 96.004 | 81.920 | 52.408 | 1.00 | 33.30 | 6 |
| ATOM | 2231 | CD2 | HIS | A | 471 | 94.892 | 82.666 | 52.352 | 1.00 | 32.78 | 6 |
| ATOM | 2232 | ND1 | HIS | A | 471 | 97.002 | 82.748 | 52.841 | 1.00 | 34.03 | 7 |
| ATOM | 2233 | CE1 | HIS | A | 471 | 96.505 | 83.964 | 53.058 | 1.00 | 33.97 | 6 |
| ATOM | 2234 | NE2 | HIS | A | 471 | 95.224 | 83.920 | 52.769 | 1.00 | 30.29 | 7 |
| ATOM | 2235 | C | HIS | A | 471 | 96.934 | 80.331 | 49.747 | 1.00 | 34.98 | 6 |
| ATOM | 2236 | O | HIS | A | 471 | 96.657 | 79.364 | 49.038 | 1.00 | 33.12 | 8 |
| ATOM | 2237 | N | ARG | A | 472 | 96.905 | 81.578 | 49.316 | 1.00 | 36.54 | 7 |
| ATOM | 2238 | CA | ARG | A | 472 | 96.585 | 81.750 | 47.924 | 1.00 | 39.87 | 6 |
| ATOM | 2239 | CB | ARG | A | 472 | 96.291 | 83.187 | 47.610 | 1.00 | 40.05 | 6 |
| ATOM | 2240 | CG | ARG | A | 472 | 96.666 | 84.052 | 48.781 | 1.00 | 41.54 | 6 |
| ATOM | 2241 | CD | ARG | A | 472 | 96.384 | 85.477 | 48.446 | 1.00 | 43.55 | 6 |
| ATOM | 2242 | NE | ARG | A | 472 | 95.082 | 85.886 | 48.902 | 1.00 | 46.69 | 7 |
| ATOM | 2243 | CZ | ARG | A | 472 | 94.809 | 86.155 | 50.168 | 1.00 | 46.61 | 6 |
| ATOM | 2244 | NH1 | ARG | A | 472 | 95.759 | 86.023 | 51.079 | 1.00 | 45.88 | 7 |
| ATOM | 2245 | NH2 | ARG | A | 472 | 93.605 | 86.557 | 50.527 | 1.00 | 46.51 | 7 |
| ATOM | 2246 | C | ARG | A | 472 | 98.053 | 81.462 | 47.961 | 1.00 | 40.61 | 6 |
| ATOM | 2247 | O | ARG | A | 472 | 98.703 | 81.615 | 48.991 | 1.00 | 41.92 | 8 |
| ATOM | 2248 | N | PHE | A | 473 | 98.574 | 81.035 | 46.839 | 1.00 | 40.52 | 7 |
| ATOM | 2249 | CA | PHE | A | 473 | 99.943 | 80.730 | 46.950 | 1.00 | 40.97 | 6 |
| ATOM | 2250 | CB | PHE | A | 473 | 99.286 | 80.277 | 45.639 | 1.00 | 42.48 | 6 |
| ATOM | 2251 | CG | PHE | A | 473 | 98.325 | 79.106 | 45.836 | 1.00 | 44.36 | 6 |
| ATOM | 2252 | CD1 | PHE | A | 473 | 97.052 | 79.185 | 46.420 | 1.00 | 45.70 | 6 |
| ATOM | 2253 | CD2 | PHE | A | 473 | 98.779 | 77.888 | 45.404 | 1.00 | 45.18 | 6 |
| ATOM | 2254 | CE1 | PHE | A | 473 | 96.272 | 78.035 | 46.510 | 1.00 | 46.58 | 6 |
| ATOM | 2255 | CE2 | PHE | A | 473 | 98.006 | 76.763 | 45.489 | 1.00 | 45.41 | 6 |
| ATOM | 2256 | CZ | PHE | A | 473 | 96.735 | 76.821 | 46.042 | 1.00 | 45.03 | 6 |
| ATOM | 2257 | C | PHE | A | 473 | 100.933 | 81.888 | 46.805 | 1.00 | 39.14 | 6 |
| ATOM | 2258 | O | PHE | A | 473 | 101.658 | 81.974 | 45.820 | 1.00 | 39.66 | 8 |
| ATOM | 2259 | N | LEU | A | 474 | 100.969 | 82.791 | 47.812 | 1.00 | 38.07 | 7 |
| ATOM | 2260 | CA | LEU | A | 474 | 101.956 | 83.853 | 47.677 | 1.00 | 37.13 | 6 |
| ATOM | 2261 | CB | LEU | A | 474 | 101.317 | 85.208 | 47.991 | 1.00 | 38.77 | 6 |
| ATOM | 2262 | CG | LEU | A | 474 | 100.047 | 85.535 | 47.201 | 1.00 | 41.22 | 6 |
| ATOM | 2263 | CD1 | LEU | A | 474 | 99.567 | 86.940 | 47.544 | 1.00 | 42.37 | 6 |
| ATOM | 2264 | CD2 | LEU | A | 474 | 100.329 | 85.413 | 45.717 | 1.00 | 40.84 | 6 |
| ATOM | 2265 | C | LEU | A | 474 | 103.147 | 83.629 | 48.603 | 1.00 | 37.43 | 6 |
| ATOM | 2266 | O | LEU | A | 474 | 102.981 | 83.241 | 49.756 | 1.00 | 36.31 | 8 |
| ATOM | 2267 | N | LEU | A | 475 | 104.346 | 83.885 | 48.089 | 1.00 | 36.57 | 7 |
| ATOM | 2268 | CA | LEU | A | 475 | 105.564 | 83.720 | 48.864 | 1.00 | 35.93 | 6 |
| ATOM | 2269 | CB | LEU | A | 475 | 106.796 | 83.845 | 47.960 | 1.00 | 35.48 | 6 |
| ATOM | 2270 | CG | LEU | A | 475 | 108.124 | 83.670 | 48.699 | 1.00 | 35.75 | 6 |
| ATOM | 2271 | CD1 | LEU | A | 475 | 108.238 | 82.220 | 49.145 | 1.00 | 35.01 | 6 |
| ATOM | 2272 | CD2 | LEU | A | 475 | 109.296 | 84.047 | 47.804 | 1.00 | 36.22 | 6 |
| ATOM | 2273 | C | LEU | A | 475 | 105.645 | 84.774 | 49.963 | 1.00 | 37.15 | 6 |
| ATOM | 2274 | O | LEU | A | 475 | 105.429 | 85.959 | 49.713 | 1.00 | 37.59 | 8 |
| ATOM | 2275 | N | ARG | A | 476 | 105.968 | 84.341 | 51.175 | 1.00 | 35.70 | 7 |
| ATOM | 2276 | CA | ARG | A | 476 | 106.071 | 85.264 | 52.294 | 1.00 | 36.99 | 6 |
| ATOM | 2277 | CB | ARG | A | 476 | 106.423 | 84.514 | 53.575 | 1.00 | 38.57 | 6 |
| ATOM | 2278 | CG | ARG | A | 476 | 105.866 | 85.158 | 54.819 | 1.00 | 41.21 | 6 |
| ATOM | 2279 | CD | ARG | A | 476 | 104.418 | 85.497 | 54.610 | 1.00 | 43.36 | 6 |
| ATOM | 2280 | NE | ARG | A | 476 | 103.529 | 84.495 | 55.177 | 1.00 | 46.52 | 7 |
| ATOM | 2281 | CZ | ARG | A | 476 | 103.207 | 84.437 | 56.463 | 1.00 | 47.41 | 6 |
| ATOM | 2282 | NH1 | ARG | A | 476 | 103.698 | 85.332 | 57.310 | 1.00 | 48.24 | 7 |
| ATOM | 2283 | NH2 | ARG | A | 476 | 102.406 | 83.476 | 56.901 | 1.00 | 49.79 | 7 |
| ATOM | 2284 | C | ARG | A | 476 | 107.157 | 86.295 | 52.005 | 1.00 | 36.41 | 6 |
| ATOM | 2285 | O | ARG | A | 476 | 108.205 | 85.957 | 51.449 | 1.00 | 34.53 | 8 |
| ATOM | 2286 | N | ARG | A | 477 | 106.915 | 87.567 | 52.404 | 1.00 | 34.99 | 7 |
| ATOM | 2287 | CA | ARG | A | 477 | 107.897 | 88.608 | 52.157 | 1.00 | 34.81 | 6 |
| ATOM | 2288 | CB | ARG | A | 477 | 107.745 | 89.165 | 50.741 | 1.00 | 37.96 | 6 |
| ATOM | 2289 | CG | ARG | A | 477 | 106.492 | 89.999 | 50.533 | 1.00 | 44.58 | 6 |
| ATOM | 2290 | CD | ARG | A | 477 | 106.282 | 90.312 | 49.061 | 1.00 | 49.78 | 6 |
| ATOM | 2291 | NE | ARG | A | 477 | 105.376 | 89.362 | 48.424 | 1.00 | 53.83 | 7 |
| ATOM | 2292 | CZ | ARG | A | 477 | 104.053 | 89.486 | 48.414 | 1.00 | 55.72 | 6 |
| ATOM | 2293 | NH1 | ARG | A | 477 | 103.481 | 90.524 | 49.009 | 1.00 | 55.12 | 7 |
| ATOM | 2294 | NH2 | ARG | A | 477 | 103.306 | 88.574 | 47.809 | 1.00 | 54.81 | 7 |
| ATOM | 2295 | C | ARG | A | 477 | 107.770 | 89.725 | 53.180 | 1.00 | 33.77 | 6 |
| ATOM | 2296 | O | ARG | A | 477 | 106.706 | 90.078 | 53.671 | 1.00 | 32.96 | 8 |
| ATOM | 2297 | N | GLY | A | 478 | 108.921 | 90.353 | 53.507 | 1.00 | 33.23 | 7 |
| ATOM | 2298 | CA | GLY | A | 478 | 108.918 | 91.462 | 54.438 | 1.00 | 34.07 | 6 |
| ATOM | 2299 | C | GLY | A | 478 | 109.615 | 91.162 | 55.741 | 1.00 | 33.63 | 6 |
| ATOM | 2300 | O | GLY | A | 478 | 110.319 | 90.152 | 55.865 | 1.00 | 31.69 | 8 |
| ATOM | 2301 | N | GLU | A | 479 | 109.430 | 92.055 | 56.708 | 1.00 | 31.25 | 7 |
| ATOM | 2302 | CA | GLU | A | 479 | 110.000 | 91.932 | 58.024 | 1.00 | 31.23 | 6 |
| ATOM | 2303 | CB | GLU | A | 479 | 110.219 | 93.291 | 58.694 | 1.00 | 32.84 | 6 |
| ATOM | 2304 | CG | GLU | A | 479 | 111.130 | 94.248 | 57.946 | 1.00 | 35.78 | 6 |
| ATOM | 2305 | CD | GLU | A | 479 | 112.606 | 93.910 | 58.029 | 1.00 | 38.07 | 6 |
| ATOM | 2306 | OE1 | GLU | A | 479 | 112.996 | 93.116 | 58.911 | 1.00 | 39.74 | 8 |
| ATOM | 2307 | OE2 | GLU | A | 479 | 113.380 | 94.450 | 57.215 | 1.00 | 39.32 | 8 |
| ATOM | 2308 | C | GLU | A | 479 | 109.070 | 91.105 | 58.855 | 1.00 | 30.28 | 6 |
| ATOM | 2309 | O | GLU | A | 479 | 107.854 | 91.071 | 58.653 | 1.00 | 31.29 | 8 |
| ATOM | 2310 | N | TYR | A | 480 | 109.666 | 90.458 | 59.775 | 1.00 | 29.09 | 7 |
| ATOM | 2311 | CA | TYR | A | 480 | 108.875 | 89.680 | 60.652 | 1.00 | 28.48 | 6 |
| ATOM | 2312 | CB | TYR | A | 480 | 108.674 | 88.275 | 60.068 | 1.00 | 28.54 | 6 |
| ATOM | 2313 | CG | TYR | A | 480 | 107.772 | 88.253 | 58.856 | 1.00 | 30.36 | 6 |
| ATOM | 2314 | CD1 | TYR | A | 480 | 108.287 | 88.380 | 57.563 | 1.00 | 32.03 | 6 |
| ATOM | 2315 | CE1 | TYR | A | 480 | 107.442 | 88.372 | 56.447 | 1.00 | 31.90 | 6 |
| ATOM | 2316 | CD2 | TYR | A | 480 | 106.388 | 88.105 | 59.002 | 1.00 | 31.14 | 6 |
| ATOM | 2317 | CE2 | TYR | A | 480 | 105.533 | 88.131 | 57.890 | 1.00 | 31.42 | 6 |
| ATOM | 2318 | CZ | TYR | A | 480 | 106.071 | 88.267 | 56.620 | 1.00 | 32.20 | 6 |
| ATOM | 2319 | OH | TYR | A | 480 | 105.235 | 88.297 | 55.527 | 1.00 | 35.64 | 8 |
| ATOM | 2320 | C | TYR | A | 480 | 109.528 | 89.681 | 62.025 | 1.00 | 28.29 | 6 |
| ATOM | 2321 | O | TYR | A | 480 | 110.747 | 89.690 | 62.146 | 1.00 | 28.45 | 8 |
| ATOM | 2322 | N | VAL | A | 481 | 108.692 | 89.661 | 63.058 | 1.00 | 27.99 | 7 |
| ATOM | 2323 | CA | VAL | A | 481 | 109.092 | 89.617 | 64.468 | 1.00 | 26.72 | 6 |
| ATOM | 2324 | CB | VAL | A | 481 | 108.874 | 90.972 | 65.177 | 1.00 | 27.39 | 6 |
| ATOM | 2325 | CG1 | VAL | A | 481 | 109.415 | 90.922 | 66.599 | 1.00 | 27.27 | 6 |
| ATOM | 2326 | CG2 | VAL | A | 481 | 109.512 | 92.115 | 64.403 | 1.00 | 27.85 | 6 |
| ATOM | 2327 | C | VAL | A | 481 | 108.237 | 88.519 | 65.094 | 1.00 | 25.68 | 6 |
| ATOM | 2328 | O | VAL | A | 481 | 107.082 | 88.757 | 65.461 | 1.00 | 21.43 | 8 |
| ATOM | 2329 | N | LEU | A | 482 | 108.818 | 87.334 | 65.203 | 1.00 | 26.79 | 7 |
| ATOM | 2330 | CA | LEU | A | 482 | 108.081 | 86.195 | 65.714 | 1.00 | 25.91 | 6 |
| ATOM | 2331 | CB | LEU | A | 482 | 108.374 | 84.983 | 64.827 | 1.00 | 26.35 | 6 |
| ATOM | 2332 | CG | LEU | A | 482 | 108.187 | 85.278 | 63.330 | 1.00 | 28.44 | 6 |
| ATOM | 2333 | CD1 | LEU | A | 482 | 108.674 | 84.100 | 62.498 | 1.00 | 30.49 | 6 |
| ATOM | 2334 | CD2 | LEU | A | 482 | 106.721 | 85.569 | 63.046 | 1.00 | 26.52 | 6 |
| ATOM | 2335 | C | LEU | A | 482 | 108.330 | 85.840 | 67.172 | 1.00 | 24.79 | 6 |
| ATOM | 2336 | O | LEU | A | 482 | 109.448 | 85.547 | 67.564 | 1.00 | 25.64 | 8 |
| ATOM | 2337 | N | HIS | A | 483 | 107.261 | 85.845 | 67.959 | 1.00 | 22.33 | 7 |
| ATOM | 2338 | CA | HIS | A | 483 | 107.341 | 85.492 | 69.373 | 1.00 | 24.80 | 6 |
| ATOM | 2339 | CB | HIS | A | 483 | 106.195 | 86.189 | 70.109 | 1.00 | 24.79 | 6 |
| ATOM | 2340 | CG | HIS | A | 483 | 106.473 | 87.640 | 70.360 | 1.00 | 27.16 | 6 |
| ATOM | 2341 | CD2 | HIS | A | 483 | 106.378 | 88.717 | 69.544 | 1.00 | 28.54 | 6 |
| ATOM | 2342 | ND1 | HIS | A | 483 | 107.044 | 88.098 | 71.532 | 1.00 | 30.66 | 7 |
| ATOM | 2343 | CE1 | HIS | A | 483 | 107.288 | 89.392 | 71.424 | 1.00 | 29.55 | 6 |
| ATOM | 2344 | NE2 | HIS | A | 483 | 106.894 | 89.792 | 70.228 | 1.00 | 30.38 | 7 |
| ATOM | 2345 | C | HIS | A | 483 | 107.290 | 83.965 | 69.434 | 1.00 | 21.99 | 6 |
| ATOM | 2346 | O | HIS | A | 483 | 106.340 | 83.329 | 68.963 | 1.00 | 22.26 | 8 |
| ATOM | 2347 | N | MET | A | 484 | 108.350 | 83.395 | 69.991 | 1.00 | 23.99 | 7 |
| ATOM | 2348 | CA | MET | A | 484 | 108.544 | 81.947 | 70.049 | 1.00 | 23.89 | 6 |
| ATOM | 2349 | CB | MET | A | 484 | 110.044 | 81.666 | 69.928 | 1.00 | 25.18 | 6 |
| ATOM | 2350 | CG | MET | A | 484 | 110.756 | 82.496 | 68.864 | 1.00 | 20.83 | 6 |
| ATOM | 2351 | SD | MET | A | 484 | 112.548 | 82.223 | 68.849 | 1.00 | 24.86 | 16 |
| ATOM | 2352 | CE | MET | A | 484 | 112.616 | 80.543 | 68.026 | 1.00 | 19.74 | 6 |
| ATOM | 2353 | C | MET | A | 484 | 107.992 | 81.159 | 71.231 | 1.00 | 24.87 | 6 |
| ATOM | 2354 | O | MET | A | 484 | 107.636 | 81.718 | 72.272 | 1.00 | 25.16 | 8 |
| ATOM | 2355 | N | TRP | A | 485 | 107.939 | 79.841 | 71.050 | 1.00 | 24.32 | 7 |
| ATOM | 2356 | CA | TRP | A | 485 | 107.460 | 78.919 | 72.073 | 1.00 | 25.36 | 6 |
| ATOM | 2357 | CB | TRP | A | 485 | 106.706 | 77.754 | 71.416 | 1.00 | 23.42 | 6 |
| ATOM | 2358 | CG | TRP | A | 485 | 105.562 | 78.184 | 70.549 | 1.00 | 21.96 | 6 |
| ATOM | 2359 | CD2 | TRP | A | 485 | 104.270 | 78.603 | 70.991 | 1.00 | 21.08 | 6 |
| ATOM | 2360 | CE2 | TRP | A | 485 | 103.509 | 78.911 | 69.839 | 1.00 | 21.15 | 6 |
| ATOM | 2361 | CE3 | TRP | A | 485 | 103.678 | 78.747 | 72.254 | 1.00 | 22.72 | 6 |
| ATOM | 2362 | CD1 | TRP | A | 485 | 105.541 | 78.253 | 69.179 | 1.00 | 20.05 | 6 |
| ATOM | 2363 | NE1 | TRP | A | 485 | 104.307 | 78.688 | 68.748 | 1.00 | 21.19 | 7 |
| ATOM | 2364 | CZ2 | TRP | A | 485 | 102.188 | 79.355 | 69.912 | 1.00 | 21.65 | 6 |
| ATOM | 2365 | CZ3 | TRP | A | 485 | 102.359 | 79.188 | 72.326 | 1.00 | 21.63 | 6 |
| ATOM | 2366 | CH2 | TRP | A | 485 | 101.631 | 79.486 | 71.160 | 1.00 | 21.41 | 6 |
| ATOM | 2367 | C | TRP | A | 485 | 108.655 | 78.368 | 72.860 | 1.00 | 24.95 | 6 |
| ATOM | 2368 | O | TRP | A | 485 | 109.663 | 77.975 | 72.268 | 1.00 | 25.32 | 8 |
| ATOM | 2369 | N | GLN | A | 486 | 108.556 | 78.345 | 74.187 | 1.00 | 29.44 | 7 |
| ATOM | 2370 | CA | GLN | A | 486 | 109.652 | 77.824 | 75.009 | 1.00 | 34.03 | 6 |
| ATOM | 2371 | CB | GLN | A | 486 | 109.752 | 78.575 | 76.349 | 1.00 | 36.27 | 6 |
| ATOM | 2372 | CG | GLN | A | 486 | 111.008 | 78.218 | 77.156 | 1.00 | 40.57 | 6 |
| ATOM | 2373 | CD | GLN | A | 486 | 111.053 | 78.841 | 78.550 | 1.00 | 43.44 | 6 |
| ATOM | 2374 | OE1 | GLN | A | 486 | 110.200 | 78.569 | 79.395 | 1.00 | 46.41 | 8 |
| ATOM | 2375 | NE2 | GLN | A | 486 | 112.058 | 79.670 | 78.795 | 1.00 | 43.21 | 7 |
| ATOM | 2376 | C | GLN | A | 486 | 109.441 | 76.335 | 75.288 | 1.00 | 37.08 | 6 |
| ATOM | 2377 | O | GLN | A | 486 | 108.345 | 75.911 | 75.653 | 1.00 | 36.27 | 8 |
| ATOM | 2378 | N | ILE | A | 487 | 110.492 | 75.543 | 75.118 | 1.00 | 39.95 | 7 |
| ATOM | 2379 | CA | ILE | A | 487 | 110.393 | 74.115 | 75.370 | 1.00 | 45.46 | 6 |
| ATOM | 2380 | CB | ILE | A | 487 | 111.398 | 73.328 | 74.505 | 1.00 | 45.86 | 6 |
| ATOM | 2381 | CG2 | ILE | A | 487 | 111.607 | 71.924 | 75.071 | 1.00 | 46.37 | 6 |
| ATOM | 2382 | CG1 | ILE | A | 487 | 110.886 | 73.287 | 73.063 | 1.00 | 46.44 | 6 |
| ATOM | 2383 | CD1 | ILE | A | 487 | 111.585 | 72.286 | 72.182 | 1.00 | 48.95 | 6 |
| ATOM | 2384 | C | ILE | A | 487 | 110.622 | 73.808 | 76.846 | 1.00 | 49.06 | 6 |
| ATOM | 2385 | O | ILE | A | 487 | 111.408 | 74.481 | 77.513 | 1.00 | 49.80 | 8 |
| ATOM | 2386 | N | SER | A | 488 | 109.923 | 72.796 | 77.348 | 1.00 | 52.78 | 7 |
| ATOM | 2387 | CA | SER | A | 488 | 110.036 | 72.398 | 78.748 | 1.00 | 57.39 | 6 |
| ATOM | 2388 | CB | SER | A | 488 | 108.642 | 72.215 | 79.351 | 1.00 | 58.31 | 6 |
| ATOM | 2389 | OG | SER | A | 488 | 107.905 | 71.242 | 78.631 | 1.00 | 61.32 | 8 |
| ATOM | 2390 | C | SER | A | 488 | 110.841 | 71.111 | 78.927 | 1.00 | 58.99 | 6 |
| ATOM | 2391 | O | SER | A | 488 | 110.646 | 70.138 | 78.198 | 1.00 | 59.80 | 8 |
| ATOM | 2392 | N | ASN | A | 498 | 106.283 | 66.282 | 68.639 | 1.00 | 44.66 | 7 |
| ATOM | 2393 | CA | ASN | A | 498 | 104.871 | 66.463 | 68.950 | 1.00 | 39.90 | 6 |
| ATOM | 2394 | CB | ASN | A | 498 | 104.692 | 66.850 | 70.418 | 1.00 | 41.65 | 6 |
| ATOM | 2395 | CG | ASN | A | 498 | 103.240 | 66.821 | 70.855 | 1.00 | 41.93 | 6 |
| ATOM | 2396 | OD1 | ASN | A | 498 | 102.836 | 67.564 | 71.749 | 1.00 | 46.35 | 8 |
| ATOM | 2397 | ND2 | ASN | A | 498 | 102.451 | 65.959 | 70.224 | 1.00 | 43.41 | 7 |
| ATOM | 2398 | C | ASN | A | 498 | 104.238 | 67.511 | 68.041 | 1.00 | 35.33 | 6 |
| ATOM | 2399 | O | ASN | A | 498 | 104.880 | 68.410 | 67.518 | 1.00 | 32.74 | 8 |
| ATOM | 2400 | N | ALA | A | 499 | 102.901 | 67.401 | 67.874 | 1.00 | 30.40 | 7 |
| ATOM | 2401 | CA | ALA | A | 499 | 102.131 | 68.312 | 67.021 | 1.00 | 27.21 | 6 |
| ATOM | 2402 | CB | ALA | A | 499 | 100.644 | 67.935 | 67.066 | 1.00 | 24.53 | 6 |
| ATOM | 2403 | C | ALA | A | 499 | 102.300 | 69.806 | 67.341 | 1.00 | 23.91 | 6 |
| ATOM | 2404 | O | ALA | A | 499 | 102.237 | 70.641 | 66.439 | 1.00 | 24.36 | 8 |
| ATOM | 2405 | N | ASP | A | 500 | 102.505 | 70.152 | 68.607 | 1.00 | 21.80 | 7 |
| ATOM | 2406 | CA | ASP | A | 500 | 102.675 | 71.565 | 68.964 | 1.00 | 25.72 | 6 |
| ATOM | 2407 | CB | ASP | A | 500 | 102.819 | 71.732 | 70.478 | 1.00 | 28.09 | 6 |
| ATOM | 2408 | CG | ASP | A | 500 | 101.494 | 71.605 | 71.200 | 1.00 | 29.75 | 6 |
| ATOM | 2409 | OD1 | ASP | A | 500 | 100.463 | 71.492 | 70.513 | 1.00 | 31.93 | 8 |
| ATOM | 2410 | OD2 | ASP | A | 500 | 101.486 | 71.623 | 72.451 | 1.00 | 31.48 | 8 |
| ATOM | 2411 | C | ASP | A | 500 | 103.873 | 72.199 | 68.260 | 1.00 | 26.30 | 6 |
| ATOM | 2412 | O | ASP | A | 500 | 103.858 | 73.387 | 67.926 | 1.00 | 25.70 | 8 |
| ATOM | 2413 | N | LYS | A | 501 | 104.903 | 71.393 | 68.022 | 1.00 | 27.34 | 7 |
| ATOM | 2414 | CA | LYS | A | 501 | 106.110 | 71.853 | 67.344 | 1.00 | 27.60 | 6 |
| ATOM | 2415 | CB | LYS | A | 501 | 107.171 | 70.750 | 67.345 | 1.00 | 31.38 | 6 |
| ATOM | 2416 | CG | LYS | A | 501 | 108.503 | 71.153 | 67.946 | 1.00 | 36.52 | 6 |
| ATOM | 2417 | CD | LYS | A | 501 | 109.658 | 70.580 | 67.161 | 1.00 | 40.10 | 6 |
| ATOM | 2418 | CE | LYS | A | 501 | 110.980 | 71.069 | 67.718 | 1.00 | 41.87 | 6 |
| ATOM | 2419 | NZ | LYS | A | 501 | 111.131 | 72.542 | 67.568 | 1.00 | 45.28 | 7 |
| ATOM | 2420 | C | LYS | A | 501 | 105.833 | 72.318 | 65.882 | 1.00 | 27.00 | 6 |
| ATOM | 2421 | O | LYS | A | 501 | 106.642 | 73.063 | 65.314 | 1.00 | 26.50 | 8 |
| ATOM | 2422 | N | LEU | A | 502 | 104.695 | 71.897 | 65.274 | 1.00 | 25.82 | 7 |
| ATOM | 2423 | CA | LEU | A | 502 | 104.335 | 72.236 | 63.883 | 1.00 | 25.80 | 6 |
| ATOM | 2424 | CB | LEU | A | 502 | 103.551 | 71.099 | 63.204 | 1.00 | 29.47 | 6 |
| ATOM | 2425 | CG | LEU | A | 502 | 104.216 | 69.719 | 63.136 | 1.00 | 31.82 | 6 |
| ATOM | 2426 | CD1 | LEU | A | 502 | 103.212 | 68.647 | 62.739 | 1.00 | 31.54 | 6 |
| ATOM | 2427 | CD2 | LEU | A | 502 | 105.397 | 69.724 | 62.173 | 1.00 | 33.26 | 6 |
| ATOM | 2428 | C | LEU | A | 502 | 103.471 | 73.452 | 63.687 | 1.00 | 26.11 | 6 |
| ATOM | 2429 | O | LEU | A | 502 | 103.340 | 73.945 | 62.561 | 1.00 | 25.95 | 8 |
| ATOM | 2430 | N | THR | A | 503 | 102.876 | 73.934 | 64.748 | 1.00 | 24.15 | 7 |
| ATOM | 2431 | CA | THR | A | 503 | 102.002 | 75.073 | 64.568 | 1.00 | 23.50 | 6 |
| ATOM | 2432 | CB | THR | A | 503 | 101.454 | 75.486 | 65.916 | 1.00 | 23.24 | 6 |
| ATOM | 2433 | OG1 | THR | A | 503 | 100.361 | 76.383 | 65.734 | 1.00 | 23.05 | 8 |
| ATOM | 2434 | CG2 | THR | A | 503 | 102.555 | 76.137 | 66.741 | 1.00 | 19.81 | 6 |
| ATOM | 2435 | C | THR | A | 503 | 102.664 | 76.262 | 63.904 | 1.00 | 23.46 | 6 |
| ATOM | 2436 | O | THR | A | 503 | 103.838 | 76.571 | 64.140 | 1.00 | 22.97 | 8 |
| ATOM | 2437 | N | SER | A | 504 | 101.859 | 76.926 | 63.050 | 1.00 | 24.06 | 7 |
| ATOM | 2438 | CA | SER | A | 504 | 102.268 | 78.167 | 62.397 | 1.00 | 24.93 | 6 |
| ATOM | 2439 | CB | SER | A | 504 | 101.622 | 78.296 | 61.009 | 1.00 | 23.19 | 6 |
| ATOM | 2440 | OG | SER | A | 504 | 100.208 | 78.251 | 61.106 | 1.00 | 27.28 | 8 |
| ATOM | 2441 | C | SER | A | 504 | 101.833 | 79.357 | 63.270 | 1.00 | 24.61 | 6 |
| ATOM | 2442 | O | SER | A | 504 | 102.098 | 80.506 | 62.934 | 1.00 | 26.18 | 8 |
| ATOM | 2443 | N | ALA | A | 505 | 101.157 | 79.087 | 64.383 | 1.00 | 25.51 | 7 |
| ATOM | 2444 | CA | ALA | A | 505 | 100.729 | 80.180 | 65.256 | 1.00 | 25.51 | 6 |
| ATOM | 2445 | CB | ALA | A | 505 | 99.589 | 79.736 | 66.157 | 1.00 | 25.12 | 6 |
| ATOM | 2446 | C | ALA | A | 505 | 101.903 | 80.631 | 66.107 | 1.00 | 25.87 | 6 |
| ATOM | 2447 | O | ALA | A | 505 | 102.813 | 79.845 | 66.396 | 1.00 | 25.27 | 8 |
| ATOM | 2448 | N | THR | A | 506 | 101.886 | 81.897 | 66.511 | 1.00 | 24.03 | 7 |
| ATOM | 2449 | CA | THR | A | 506 | 102.958 | 82.422 | 67.353 | 1.00 | 24.84 | 6 |
| ATOM | 2450 | CB | THR | A | 506 | 103.450 | 83.798 | 66.856 | 1.00 | 23.83 | 6 |
| ATOM | 2451 | OG1 | THR | A | 506 | 102.335 | 84.682 | 66.730 | 1.00 | 21.21 | 8 |
| ATOM | 2452 | CG2 | THR | A | 506 | 104.152 | 83.657 | 65.510 | 1.00 | 23.58 | 6 |
| ATOM | 2453 | C | THR | A | 506 | 102.491 | 82.543 | 68.805 | 1.00 | 23.71 | 6 |
| ATOM | 2454 | O | THR | A | 506 | 101.293 | 82.500 | 69.089 | 1.00 | 25.70 | 8 |
| ATOM | 2455 | N | ASN | A | 507 | 103.447 | 82.707 | 69.718 | 1.00 | 23.90 | 7 |
| ATOM | 2456 | CA | ASN | A | 507 | 103.152 | 82.846 | 71.142 | 1.00 | 24.41 | 6 |
| ATOM | 2457 | CB | ASN | A | 507 | 104.463 | 82.771 | 71.931 | 1.00 | 23.91 | 6 |
| ATOM | 2458 | CG | ASN | A | 507 | 104.260 | 82.549 | 73.408 | 1.00 | 24.42 | 6 |
| ATOM | 2459 | OD1 | ASN | A | 507 | 103.272 | 82.989 | 73.994 | 1.00 | 24.62 | 8 |
| ATOM | 2460 | ND2 | ASN | A | 507 | 105.204 | 81.843 | 74.022 | 1.00 | 23.40 | 7 |
| ATOM | 2461 | C | ASN | A | 507 | 102.409 | 84.117 | 71.496 | 1.00 | 24.10 | 6 |
| ATOM | 2462 | O | ASN | A | 507 | 102.877 | 85.215 | 71.222 | 1.00 | 25.78 | 8 |
| ATOM | 2463 | N | PRO | A | 508 | 101.219 | 83.948 | 72.106 | 1.00 | 25.79 | 7 |
| ATOM | 2464 | CD | PRO | A | 508 | 100.345 | 82.862 | 71.606 | 1.00 | 25.89 | 6 |
| ATOM | 2465 | CA | PRO | A | 508 | 100.300 | 85.085 | 72.484 | 1.00 | 29.11 | 6 |
| ATOM | 2466 | CB | PRO | A | 508 | 99.101 | 84.368 | 73.049 | 1.00 | 26.98 | 6 |
| ATOM | 2467 | CG | PRO | A | 508 | 98.997 | 83.168 | 72.168 | 1.00 | 29.40 | 6 |
| ATOM | 2468 | C | PRO | A | 508 | 100.917 | 86.165 | 73.313 | 1.00 | 30.58 | 6 |
| ATOM | 2469 | O | PRO | A | 508 | 100.694 | 67.361 | 73.133 | 1.00 | 29.18 | 8 |
| ATOM | 2470 | N | ASP | A | 509 | 101.689 | 85.687 | 74.247 | 1.00 | 32.03 | 7 |
| ATOM | 2471 | CA | ASP | A | 509 | 102.380 | 86.494 | 75.208 | 1.00 | 34.03 | 6 |
| ATOM | 2472 | CB | ASP | A | 509 | 102.944 | 85.609 | 76.323 | 1.00 | 37.27 | 6 |
| ATOM | 2473 | CG | ASP | A | 509 | 103.456 | 86.406 | 77.509 | 1.00 | 41.74 | 6 |
| ATOM | 2474 | OD1 | ASP | A | 509 | 103.765 | 87.597 | 77.335 | 1.00 | 41.44 | 8 |
| ATOM | 2475 | OD2 | ASP | A | 509 | 103.533 | 85.827 | 78.618 | 1.00 | 44.22 | 8 |
| ATOM | 2476 | C | ASP | A | 509 | 103.508 | 87.298 | 74.567 | 1.00 | 33.79 | 6 |
| ATOM | 2477 | O | ASP | A | 509 | 104.646 | 86.846 | 74.510 | 1.00 | 35.02 | 8 |
| ATOM | 2478 | N | LYS | A | 510 | 103.229 | 88.490 | 74.078 | 1.00 | 34.74 | 7 |
| ATOM | 2479 | CA | LYS | A | 510 | 104.249 | 89.307 | 73.433 | 1.00 | 37.91 | 6 |
| ATOM | 2480 | CB | LYS | A | 510 | 103.569 | 90.275 | 72.434 | 1.00 | 39.34 | 6 |
| ATOM | 2481 | CG | LYS | A | 510 | 103.243 | 89.663 | 71.068 | 1.00 | 41.56 | 6 |
| ATOM | 2482 | CD | LYS | A | 510 | 102.048 | 88.706 | 71.136 | 1.00 | 43.30 | 6 |
| ATOM | 2483 | CE | LYS | A | 510 | 101.556 | 88.350 | 69.739 | 1.00 | 44.13 | 6 |
| ATOM | 2484 | NZ | LYS | A | 510 | 102.452 | 87.378 | 69.063 | 1.00 | 44.47 | 7 |
| ATOM | 2485 | C | LYS | A | 510 | 105.097 | 90.124 | 74.411 | 1.00 | 38.87 | 6 |
| ATOM | 2486 | O | LYS | A | 510 | 105.957 | 90.890 | 73.982 | 1.00 | 39.40 | 8 |
| ATOM | 2487 | N | GLU | A | 511 | 104.847 | 89.996 | 75.701 | 1.00 | 40.90 | 7 |
| ATOM | 2488 | CA | GLU | A | 511 | 105.522 | 90.794 | 76.718 | 1.00 | 41.77 | 6 |
| ATOM | 2489 | CB | GLU | A | 511 | 104.473 | 91.348 | 77.687 | 1.00 | 44.02 | 6 |
| ATOM | 2490 | CG | GLU | A | 511 | 103.239 | 91.887 | 76.985 | 1.00 | 49.38 | 6 |
| ATOM | 2491 | CD | GLU | A | 511 | 102.012 | 91.948 | 77.876 | 1.00 | 53.25 | 6 |
| ATOM | 2492 | OE1 | GLU | A | 511 | 101.383 | 93.028 | 77.954 | 1.00 | 55.24 | 8 |
| ATOM | 2493 | OE2 | GLU | A | 511 | 101.673 | 90.913 | 78.493 | 1.00 | 55.46 | 8 |
| ATOM | 2494 | C | GLU | A | 511 | 106.612 | 90.032 | 77.482 | 1.00 | 40.78 | 6 |
| ATOM | 2495 | O | GLU | A | 511 | 107.492 | 90.622 | 78.090 | 1.00 | 39.95 | 8 |
| ATOM | 2496 | N | ASN | A | 512 | 106.548 | 88.698 | 77.427 | 1.00 | 39.88 | 7 |
| ATOM | 2497 | CA | ASN | A | 512 | 107.503 | 87.869 | 78.151 | 1.00 | 39.87 | 6 |
| ATOM | 2498 | CB | ASN | A | 512 | 106.803 | 87.049 | 79.195 | 1.00 | 41.09 | 6 |
| ATOM | 2499 | CG | ASN | A | 512 | 106.067 | 87.919 | 80.149 | 1.00 | 40.94 | 6 |
| ATOM | 2500 | OD1 | ASN | A | 512 | 106.684 | 88.675 | 80.904 | 1.00 | 43.47 | 8 |
| ATOM | 2501 | ND2 | ASN | A | 512 | 104.743 | 87.817 | 80.140 | 1.00 | 41.72 | 7 |
| ATOM | 2502 | C | ASN | A | 512 | 108.232 | 86.865 | 77.295 | 1.00 | 39.20 | 6 |
| ATOM | 2503 | O | ASN | A | 512 | 109.200 | 86.239 | 77.737 | 1.00 | 39.14 | 8 |
| ATOM | 2504 | N | SER | A | 513 | 107.766 | 86.705 | 76.082 | 1.00 | 37.70 | 7 |
| ATOM | 2505 | CA | SER | A | 513 | 108.364 | 85.672 | 75.270 | 1.00 | 36.22 | 6 |
| ATOM | 2506 | CB | SER | A | 513 | 107.329 | 85.170 | 74.258 | 1.00 | 37.61 | 6 |
| ATOM | 2507 | OG | SER | A | 513 | 106.189 | 86.016 | 74.248 | 1.00 | 40.03 | 8 |
| ATOM | 2508 | C | SER | A | 513 | 109.600 | 86.076 | 74.522 | 1.00 | 34.54 | 6 |
| ATOM | 2509 | O | SER | A | 513 | 109.913 | 87.255 | 74.358 | 1.00 | 31.23 | 8 |
| ATOM | 2510 | N | MET | A | 514 | 110.310 | 85.054 | 74.066 | 1.00 | 31.81 | 7 |
| ATOM | 2511 | CA | MET | A | 514 | 111.483 | 85.262 | 73.236 | 1.00 | 30.54 | 6 |
| ATOM | 2512 | CB | MET | A | 514 | 112.305 | 83.966 | 73.147 | 1.00 | 31.80 | 6 |
| ATOM | 2513 | CG | MET | A | 514 | 113.384 | 83.954 | 72.065 | 1.00 | 32.96 | 6 |
| ATOM | 2514 | SD | MET | A | 514 | 114.680 | 85.192 | 72.299 | 1.00 | 35.28 | 16 |
| ATOM | 2515 | CE | MET | A | 514 | 115.720 | 84.347 | 73.523 | 1.00 | 34.70 | 6 |
| ATOM | 2516 | C | MET | A | 514 | 110.949 | 85.631 | 71.858 | 1.00 | 29.07 | 6 |
| ATOM | 2517 | O | MET | A | 514 | 109.900 | 85.138 | 71.443 | 1.00 | 27.75 | 8 |
| ATOM | 2518 | N | SER | A | 515 | 111.649 | 86.514 | 71.156 | 1.00 | 29.25 | 7 |
| ATOM | 2519 | CA | SER | A | 515 | 111.222 | 86.883 | 69.813 | 1.00 | 29.37 | 6 |
| ATOM | 2520 | CB | SER | A | 515 | 110.645 | 88.302 | 69.792 | 1.00 | 28.56 | 6 |
| ATOM | 2521 | OG | SER | A | 515 | 111.669 | 89.260 | 69.984 | 1.00 | 29.13 | 8 |
| ATOM | 2522 | C | SER | A | 515 | 112.416 | 86.798 | 68.870 | 1.00 | 28.19 | 6 |
| ATOM | 2523 | O | SER | A | 515 | 113.569 | 86.935 | 69.294 | 1.00 | 26.89 | 8 |
| ATOM | 2524 | N | ILE | A | 516 | 112.135 | 86.545 | 67.595 | 1.00 | 26.12 | 7 |
| ATOM | 2525 | CA | ILE | A | 516 | 113.186 | 86.467 | 66.585 | 1.00 | 24.23 | 6 |
| ATOM | 2526 | CB | ILE | A | 516 | 113.443 | 85.001 | 66.129 | 1.00 | 23.37 | 6 |
| ATOM | 2527 | CG2 | ILE | A | 516 | 112.187 | 84.421 | 65.506 | 1.00 | 20.37 | 6 |
| ATOM | 2528 | CG1 | ILE | A | 516 | 114.625 | 84.952 | 65.153 | 1.00 | 21.87 | 6 |
| ATOM | 2529 | CD1 | ILE | A | 516 | 115.063 | 83.520 | 64.767 | 1.00 | 21.61 | 6 |
| ATOM | 2530 | C | ILE | A | 516 | 112.758 | 87.317 | 65.392 | 1.00 | 24.93 | 6 |
| ATOM | 2531 | O | ILE | A | 516 | 111.647 | 87.181 | 64.874 | 1.00 | 23.44 | 8 |
| ATOM | 2532 | N | SER | A | 517 | 113.650 | 88.206 | 64.970 | 1.00 | 24.79 | 7 |
| ATOM | 2533 | CA | SER | A | 517 | 113.378 | 89.098 | 63.861 | 1.00 | 25.17 | 6 |
| ATOM | 2534 | CB | SER | A | 517 | 113.835 | 90.517 | 64.224 | 1.00 | 25.01 | 6 |
| ATOM | 2535 | OG | SER | A | 517 | 113.227 | 90.927 | 65.438 | 1.00 | 29.91 | 8 |
| ATOM | 2536 | C | SER | A | 517 | 114.104 | 88.631 | 62.615 | 1.00 | 24.52 | 6 |
| ATOM | 2537 | O | SER | A | 517 | 115.309 | 88.360 | 62.652 | 1.00 | 26.27 | 8 |
| ATOM | 2538 | N | ILE | A | 518 | 113.376 | 88.545 | 61.511 | 1.00 | 23.76 | 7 |
| ATOM | 2539 | CA | ILE | A | 518 | 113.967 | 88.121 | 60.245 | 1.00 | 23.15 | 6 |
| ATOM | 2540 | CB | ILE | A | 518 | 113.581 | 86.651 | 59.895 | 1.00 | 21.98 | 6 |
| ATOM | 2541 | CG2 | ILE | A | 518 | 114.065 | 85.685 | 61.004 | 1.00 | 23.34 | 6 |
| ATOM | 2542 | CG1 | ILE | A | 518 | 112.068 | 86.557 | 59.671 | 1.00 | 21.53 | 6 |
| ATOM | 2543 | CD1 | ILE | A | 518 | 111.581 | 85.190 | 59.129 | 1.00 | 23.43 | 6 |
| ATOM | 2544 | C | ILE | A | 518 | 113.453 | 89.006 | 59.106 | 1.00 | 26.69 | 6 |
| ATOM | 2545 | O | ILE | A | 518 | 112.462 | 89.726 | 59.266 | 1.00 | 23.93 | 8 |
| ATOM | 2546 | N | LEU | A | 519 | 114.135 | 88.930 | 57.964 | 1.00 | 26.46 | 7 |
| ATOM | 2547 | CA | LEU | A | 519 | 113.753 | 89.660 | 56.761 | 1.00 | 28.71 | 6 |
| ATOM | 2548 | CB | LEU | A | 519 | 114.785 | 90.736 | 56.396 | 1.00 | 29.16 | 6 |
| ATOM | 2549 | CG | LEU | A | 519 | 114.562 | 91.354 | 55.004 | 1.00 | 28.15 | 6 |
| ATOM | 2550 | CD1 | LEU | A | 519 | 113.194 | 92.007 | 54.935 | 1.00 | 30.77 | 6 |
| ATOM | 2551 | CD2 | LEU | A | 519 | 115.648 | 92.369 | 54.704 | 1.00 | 32.15 | 6 |
| ATOM | 2552 | C | LEU | A | 519 | 113.658 | 88.675 | 55.601 | 1.00 | 30.98 | 6 |
| ATOM | 2553 | O | LEU | A | 519 | 114.567 | 87.878 | 55.376 | 1.00 | 28.40 | 8 |
| ATOM | 2554 | N | LEU | A | 520 | 112.479 | 88.709 | 54.978 | 1.00 | 34.09 | 7 |
| ATOM | 2555 | CA | LEU | A | 520 | 112.186 | 87.841 | 53.869 | 1.00 | 39.00 | 6 |
| ATOM | 2556 | CB | LEU | A | 520 | 110.882 | 87.073 | 54.065 | 1.00 | 36.64 | 6 |
| ATOM | 2557 | CG | LEU | A | 520 | 111.124 | 85.608 | 54.379 | 1.00 | 38.86 | 6 |
| ATOM | 2558 | CD1 | LEU | A | 520 | 112.223 | 85.440 | 55.426 | 1.00 | 38.04 | 6 |
| ATOM | 2559 | CD2 | LEU | A | 520 | 109.837 | 84.953 | 54.868 | 1.00 | 35.20 | 6 |
| ATOM | 2560 | C | LEU | A | 520 | 112.193 | 88.732 | 52.669 | 1.00 | 43.86 | 6 |
| ATOM | 2561 | O | LEU | A | 520 | 111.458 | 89.715 | 52.521 | 1.00 | 44.46 | 8 |
| ATOM | 2562 | N | ASP | A | 521 | 113.087 | 88.311 | 51.813 | 1.00 | 49.55 | 7 |
| ATOM | 2563 | CA | ASP | A | 521 | 113.431 | 89.014 | 50.614 | 1.00 | 54.26 | 6 |
| ATOM | 2564 | CB | ASP | A | 521 | 114.476 | 88.255 | 49.833 | 1.00 | 57.21 | 6 |
| ATOM | 2565 | CG | ASP | A | 521 | 114.723 | 89.013 | 48.582 | 1.00 | 59.63 | 6 |
| ATOM | 2566 | OD1 | ASP | A | 521 | 114.909 | 90.249 | 48.650 | 1.00 | 60.15 | 8 |
| ATOM | 2567 | OD2 | ASP | A | 521 | 114.742 | 88.362 | 47.516 | 1.00 | 61.26 | 8 |
| ATOM | 2568 | C | ASP | A | 521 | 112.304 | 89.386 | 49.678 | 1.00 | 55.67 | 6 |
| ATOM | 2569 | O | ASP | A | 521 | 111.833 | 88.605 | 48.845 | 1.00 | 55.90 | 8 |
| ATOM | 2570 | N | ASN | A | 522 | 111.936 | 90.693 | 49.629 | 1.00 | 57.59 | 7 |
| ATOM | 2571 | CA | ASN | A | 522 | 110.811 | 91.358 | 48.821 | 1.00 | 59.08 | 6 |
| ATOM | 2572 | CB | ASN | A | 522 | 110.686 | 92.763 | 49.372 | 1.00 | 60.15 | 6 |
| ATOM | 2573 | CG | ASN | A | 522 | 109.322 | 92.927 | 49.935 | 1.00 | 60.28 | 6 |
| ATOM | 2574 | OD1 | ASN | A | 522 | 108.399 | 92.209 | 49.528 | 1.00 | 60.62 | 8 |
| ATOM | 2575 | ND2 | ASN | A | 522 | 109.160 | 93.850 | 50.877 | 1.00 | 61.71 | 7 |
| ATOM | 2576 | C | ASN | A | 522 | 110.766 | 91.464 | 47.230 | 1.00 | 59.95 | 6 |
| ATOM | 2577 | O | ASN | A | 522 | 111.621 | 90.912 | 46.522 | 1.00 | 60.83 | 8 |
| ATOM | 2578 | N | TYR | A | 523 | 109.719 | 92.186 | 46.757 | 1.00 | 20.00 | 7 |
| ATOM | 2579 | CA | TYR | A | 523 | 109.455 | 92.305 | 45.342 | 1.00 | 20.00 | 6 |
| ATOM | 2580 | C | TYR | A | 523 | 108.573 | 93.429 | 44.862 | 1.00 | 20.00 | 6 |
| ATOM | 2581 | O | TYR | A | 523 | 107.591 | 93.857 | 45.471 | 1.00 | 20.00 | 8 |
| ATOM | 2582 | CB | TYR | A | 523 | 108.907 | 90.999 | 44.845 | 1.00 | 20.00 | 6 |
| ATOM | 2583 | CG | TYR | A | 523 | 109.926 | 89.975 | 45.181 | 1.00 | 20.00 | 6 |
| ATOM | 2584 | CD1 | TYR | A | 523 | 109.572 | 88.714 | 45.629 | 1.00 | 20.00 | 6 |
| ATOM | 2585 | CD2 | TYR | A | 523 | 111.271 | 90.331 | 45.048 | 1.00 | 20.00 | 6 |
| ATOM | 2586 | CE1 | TYR | A | 523 | 110.554 | 87.834 | 45.996 | 1.00 | 20.00 | 6 |
| ATOM | 2587 | CE2 | TYR | A | 523 | 112.261 | 89.458 | 45.411 | 1.00 | 20.00 | 6 |
| ATOM | 2588 | CZ | TYR | A | 523 | 111.882 | 88.211 | 45.885 | 1.00 | 20.00 | 6 |
| ATOM | 2589 | OH | TYR | A | 523 | 112.865 | 87.307 | 46.248 | 1.00 | 20.00 | 8 |
| ATOM | 2590 | N | CYS | A | 524 | 108.686 | 93.646 | 43.523 | 1.00 | 20.00 | 7 |
| ATOM | 2591 | CA | CYS | A | 524 | 107.834 | 94.513 | 42.647 | 1.00 | 20.00 | 6 |
| ATOM | 2592 | C | CYS | A | 524 | 106.325 | 94.079 | 42.549 | 1.00 | 20.00 | 6 |
| ATOM | 2593 | O | CYS | A | 524 | 106.071 | 92.887 | 42.489 | 1.00 | 20.00 | 8 |
| ATOM | 2594 | CB | CYS | A | 524 | 108.394 | 94.465 | 41.239 | 1.00 | 20.00 | 6 |
| ATOM | 2595 | SG | CYS | A | 524 | 109.146 | 96.025 | 40.688 | 1.00 | 20.00 | 16 |
| ATOM | 2596 | N | HIS | A | 525 | 105.339 | 95.015 | 42.525 | 1.00 | 20.00 | 7 |
| ATOM | 2597 | CA | HIS | A | 525 | 103.890 | 94.619 | 42.360 | 1.00 | 20.00 | 6 |
| ATOM | 2598 | C | HIS | A | 525 | 102.875 | 95.667 | 41.947 | 1.00 | 20.00 | 6 |
| ATOM | 2599 | O | HIS | A | 525 | 102.911 | 96.791 | 42.439 | 1.00 | 20.00 | 8 |
| ATOM | 2600 | CB | HIS | A | 525 | 103.070 | 94.434 | 43.623 | 1.00 | 20.00 | 6 |
| ATOM | 2601 | CG | HIS | A | 525 | 103.537 | 93.702 | 44.833 | 1.00 | 20.00 | 6 |
| ATOM | 2602 | ND1 | HIS | A | 525 | 103.057 | 92.455 | 45.084 | 1.00 | 20.00 | 7 |
| ATOM | 2603 | CD2 | HIS | A | 525 | 104.359 | 94.034 | 45.860 | 1.00 | 20.00 | 6 |
| ATOM | 2604 | CE1 | HIS | A | 525 | 103.550 | 92.008 | 46.229 | 1.00 | 20.00 | 6 |
| ATOM | 2605 | NE2 | HIS | A | 525 | 104.341 | 92.944 | 46.712 | 1.00 | 20.00 | 7 |
| ATOM | 2606 | N | PRO | A | 526 | 101.983 | 95.299 | 41.049 | 1.00 | 20.00 | 7 |
| ATOM | 2607 | CA | PRO | A | 526 | 100.817 | 96.132 | 40.874 | 1.00 | 20.00 | 6 |
| ATOM | 2608 | C | PRO | A | 526 | 99.895 | 96.178 | 42.075 | 1.00 | 20.00 | 6 |
| ATOM | 2609 | O | PRO | A | 526 | 100.143 | 95.651 | 43.156 | 1.00 | 20.00 | 8 |
| ATOM | 2610 | CB | PRO | A | 526 | 100.164 | 95.598 | 39.633 | 1.00 | 20.00 | 6 |
| ATOM | 2611 | CG | PRO | A | 526 | 101.334 | 95.289 | 38.783 | 1.00 | 20.00 | 6 |
| ATOM | 2612 | CD | PRO | A | 526 | 102.523 | 95.122 | 39.679 | 1.00 | 20.00 | 6 |
| ATOM | 2613 | N | ILE | A | 527 | 98.793 | 96.870 | 41.784 | 1.00 | 20.00 | 7 |
| ATOM | 2614 | CA | ILE | A | 527 | 97.609 | 96.990 | 42.603 | 1.00 | 20.00 | 6 |
| ATOM | 2615 | C | ILE | A | 527 | 96.458 | 96.887 | 41.606 | 1.00 | 20.00 | 6 |
| ATOM | 2616 | O | ILE | A | 527 | 96.678 | 96.793 | 40.397 | 1.00 | 20.00 | 8 |
| ATOM | 2617 | CB | ILE | A | 527 | 97.460 | 98.333 | 43.378 | 1.00 | 20.00 | 6 |
| ATOM | 2618 | CG1 | ILE | A | 527 | 98.553 | 98.476 | 44.438 | 1.00 | 20.00 | 6 |
| ATOM | 2619 | CG2 | ILE | A | 527 | 96.079 | 98.445 | 44.015 | 1.00 | 20.00 | 6 |
| ATOM | 2620 | CD1 | ILE | A | 527 | 98.590 | 99.835 | 45.100 | 1.00 | 20.00 | 6 |
| ATOM | 2621 | N | TRP | A | 617 | 91.176 | 53.634 | 43.913 | 1.00 | 45.70 | 7 |
| ATOM | 2622 | CA | TRP | A | 617 | 91.003 | 54.192 | 45.249 | 1.00 | 44.93 | 6 |
| ATOM | 2623 | CB | TRP | A | 617 | 91.940 | 55.386 | 45.444 | 1.00 | 43.14 | 6 |
| ATOM | 2624 | CG | TRP | A | 617 | 91.701 | 56.136 | 46.720 | 1.00 | 42.60 | 6 |
| ATOM | 2625 | CD2 | TRP | A | 617 | 92.221 | 55.813 | 48.011 | 1.00 | 41.19 | 6 |
| ATOM | 2626 | CE2 | TRP | A | 617 | 91.746 | 56.795 | 48.910 | 1.00 | 41.70 | 6 |
| ATOM | 2627 | CE3 | TRP | A | 617 | 93.044 | 54.789 | 48.500 | 1.00 | 41.13 | 6 |
| ATOM | 2628 | CD1 | TRP | A | 617 | 90.948 | 57.264 | 46.879 | 1.00 | 40.98 | 6 |
| ATOM | 2629 | NE1 | TRP | A | 617 | 90.972 | 57.668 | 48.192 | 1.00 | 41.86 | 7 |
| ATOM | 2630 | CZ2 | TRP | A | 617 | 92.070 | 56.785 | 50.273 | 1.00 | 41.18 | 6 |
| ATOM | 2631 | CZ3 | TRP | A | 617 | 93.365 | 54.779 | 49.856 | 1.00 | 41.43 | 6 |
| ATOM | 2632 | CH2 | TRP | A | 617 | 92.878 | 55.773 | 50.727 | 1.00 | 39.94 | 6 |
| ATOM | 2633 | C | TRP | A | 617 | 89.565 | 54.619 | 45.510 | 1.00 | 45.29 | 6 |
| ATOM | 2634 | O | TRP | A | 617 | 89.002 | 54.324 | 46.566 | 1.00 | 45.16 | 8 |
| ATOM | 2635 | N | ASP | A | 618 | 88.977 | 55.309 | 44.539 | 1.00 | 47.06 | 7 |
| ATOM | 2636 | CA | ASP | A | 618 | 87.606 | 55.781 | 44.657 | 1.00 | 49.28 | 6 |
| ATOM | 2637 | CB | ASP | A | 618 | 87.225 | 56.609 | 43.424 | 1.00 | 50.80 | 6 |
| ATOM | 2638 | CG | ASP | A | 618 | 88.207 | 57.739 | 43.145 | 1.00 | 53.47 | 6 |
| ATOM | 2639 | OD1 | ASP | A | 618 | 88.568 | 58.470 | 44.089 | 1.00 | 53.71 | 8 |
| ATOM | 2640 | OD2 | ASP | A | 618 | 88.612 | 57.907 | 41.975 | 1.00 | 54.75 | 8 |
| ATOM | 2641 | C | ASP | A | 618 | 86.606 | 54.635 | 44.823 | 1.00 | 49.55 | 6 |
| ATOM | 2642 | O | ASP | A | 618 | 85.541 | 54.817 | 45.414 | 1.00 | 49.38 | 8 |
| ATOM | 2643 | N | SER | A | 620 | 82.160 | 47.834 | 53.597 | 1.00 | 120.04 | 7 |
| ATOM | 2644 | CA | SER | A | 620 | 83.467 | 48.156 | 54.184 | 1.00 | 120.04 | 6 |
| ATOM | 2645 | CB | SER | A | 620 | 84.478 | 48.514 | 53.091 | 1.00 | 114.87 | 6 |
| ATOM | 2646 | OG | SER | A | 620 | 84.542 | 47.499 | 52.106 | 1.00 | 114.87 | 8 |
| ATOM | 2647 | C | SER | A | 620 | 83.268 | 49.275 | 55.216 | 1.00 | 120.04 | 6 |
| ATOM | 2648 | O | SER | A | 620 | 82.527 | 50.231 | 54.932 | 1.00 | 120.04 | 8 |
| ATOM | 2649 | N | ALA | A | 621 | 83.799 | 49.215 | 56.414 | 1.00 | 126.83 | 7 |
| ATOM | 2650 | CA | ALA | A | 621 | 83.421 | 50.284 | 57.320 | 1.00 | 126.83 | 6 |
| ATOM | 2651 | CB | ALA | A | 621 | 82.262 | 49.848 | 58.202 | 1.00 | 53.16 | 6 |
| ATOM | 2652 | C | ALA | A | 621 | 84.568 | 50.738 | 58.190 | 1.00 | 126.83 | 6 |
| ATOM | 2653 | O | ALA | A | 621 | 84.907 | 51.909 | 58.269 | 1.00 | 126.83 | 8 |
| ATOM | 2654 | N | LEU | A | 622 | 85.180 | 49.756 | 58.837 | 1.00 | 87.63 | 7 |
| ATOM | 2655 | CA | LEU | A | 622 | 86.364 | 50.000 | 59.626 | 1.00 | 85.57 | 6 |
| ATOM | 2656 | CB | LEU | A | 622 | 86.603 | 48.852 | 60.581 | 1.00 | 104.76 | 6 |
| ATOM | 2657 | CG | LEU | A | 622 | 85.448 | 48.518 | 61.511 | 1.00 | 105.42 | 6 |
| ATOM | 2658 | CD1 | LEU | A | 622 | 85.788 | 47.329 | 62.386 | 1.00 | 104.68 | 6 |
| ATOM | 2659 | CD2 | LEU | A | 622 | 85.080 | 49.734 | 62.347 | 1.00 | 107.13 | 6 |
| ATOM | 2660 | C | LEU | A | 622 | 87.556 | 50.264 | 58.687 | 1.00 | 84.15 | 6 |
| ATOM | 2661 | O | LEU | A | 622 | 88.403 | 51.100 | 58.993 | 1.00 | 82.55 | 8 |
| ATOM | 2662 | N | ASP | A | 623 | 87.626 | 49.553 | 57.545 | 1.00 | 63.77 | 7 |
| ATOM | 2663 | CA | ASP | A | 623 | 88.701 | 49.751 | 56.577 | 1.00 | 63.34 | 6 |
| ATOM | 2664 | CB | ASP | A | 623 | 88.361 | 49.112 | 55.239 | 1.00 | 133.05 | 6 |
| ATOM | 2665 | CG | ASP | A | 623 | 88.051 | 47.674 | 55.436 | 1.00 | 135.81 | 6 |
| ATOM | 2666 | OD1 | ASP | A | 623 | 88.558 | 47.085 | 56.420 | 1.00 | 136.27 | 8 |
| ATOM | 2667 | OD2 | ASP | A | 623 | 87.299 | 47.119 | 54.624 | 1.00 | 137.84 | 8 |
| ATOM | 2668 | C | ASP | A | 623 | 88.919 | 51.205 | 56.382 | 1.00 | 63.15 | 6 |
| ATOM | 2669 | O | ASP | A | 623 | 90.037 | 51.702 | 56.410 | 1.00 | 60.15 | 8 |
| ATOM | 2670 | N | VAL | A | 624 | 87.814 | 51.866 | 56.195 | 1.00 | 62.81 | 7 |
| ATOM | 2671 | CA | VAL | A | 624 | 87.841 | 53.278 | 56.030 | 1.00 | 60.77 | 6 |
| ATOM | 2672 | CB | VAL | A | 624 | 86.427 | 53.751 | 55.804 | 1.00 | 146.44 | 6 |
| ATOM | 2673 | CG1 | VAL | A | 624 | 86.368 | 55.269 | 55.723 | 1.00 | 145.95 | 6 |
| ATOM | 2674 | CG2 | VAL | A | 624 | 85.851 | 53.120 | 54.543 | 1.00 | 146.22 | 6 |
| ATOM | 2675 | C | VAL | A | 624 | 88.489 | 53.984 | 57.213 | 1.00 | 60.38 | 6 |
| ATOM | 2676 | O | VAL | A | 624 | 88.758 | 55.178 | 57.142 | 1.00 | 60.38 | 8 |
| ATOM | 2677 | N | GLY | A | 625 | 88.755 | 53.272 | 58.289 | 1.00 | 127.53 | 7 |
| ATOM | 2678 | CA | GLY | A | 625 | 89.402 | 53.958 | 59.396 | 1.00 | 127.53 | 6 |
| ATOM | 2679 | C | GLY | A | 625 | 90.868 | 54.234 | 59.044 | 1.00 | 127.53 | 6 |
| ATOM | 2680 | O | GLY | A | 625 | 91.509 | 55.141 | 59.566 | 1.00 | 127.53 | 8 |
| ATOM | 2681 | N | LEU | A | 626 | 91.366 | 53.394 | 58.148 | 1.00 | 56.23 | 7 |
| ATOM | 2682 | CA | LEU | A | 626 | 92.752 | 53.437 | 57.671 | 1.00 | 56.23 | 6 |
| ATOM | 2683 | CB | LEU | A | 626 | 93.111 | 52.137 | 56.940 | 1.00 | 197.30 | 6 |
| ATOM | 2684 | CG | LEU | A | 626 | 93.013 | 50.846 | 57.755 | 1.00 | 198.37 | 6 |
| ATOM | 2685 | CD1 | LEU | A | 626 | 93.390 | 49.643 | 56.903 | 1.00 | 197.30 | 6 |
| ATOM | 2686 | CD2 | LEU | A | 626 | 93.896 | 50.923 | 58.990 | 1.00 | 198.98 | 6 |
| ATOM | 2687 | C | LEU | A | 626 | 92.966 | 54.664 | 56.809 | 1.00 | 56.23 | 6 |
| ATOM | 2688 | O | LEU | A | 626 | 94.071 | 54.994 | 56.401 | 1.00 | 56.23 | 8 |
| ATOM | 2689 | N | THR | A | 627 | 91.878 | 55.333 | 56.609 | 1.00 | 53.16 | 7 |
| ATOM | 2690 | CA | THR | A | 627 | 91.834 | 56.585 | 55.904 | 1.00 | 53.16 | 6 |
| ATOM | 2691 | CB | THR | A | 627 | 90.379 | 56.924 | 55.569 | 1.00 | 152.09 | 6 |
| ATOM | 2692 | OG1 | THR | A | 627 | 89.832 | 55.914 | 54.715 | 1.00 | 153.90 | 8 |
| ATOM | 2693 | CG2 | THR | A | 627 | 90.310 | 58.287 | 54.895 | 1.00 | 152.95 | 6 |
| ATOM | 2694 | C | THR | A | 627 | 92.481 | 57.650 | 56.802 | 1.00 | 53.16 | 6 |
| ATOM | 2695 | O | THR | A | 627 | 92.631 | 58.792 | 56.408 | 1.00 | 53.16 | 8 |
| ATOM | 2696 | N | MET | A | 628 | 92.873 | 57.240 | 58.039 | 1.00 | 26.65 | 7 |
| ATOM | 2697 | CA | MET | A | 628 | 93.499 | 58.112 | 59.089 | 1.00 | 27.89 | 6 |
| ATOM | 2698 | CB | MET | A | 628 | 93.286 | 57.492 | 60.468 | 1.00 | 29.54 | 6 |
| ATOM | 2699 | CG | MET | A | 628 | 91.830 | 57.358 | 60.843 | 1.00 | 38.86 | 6 |
| ATOM | 2700 | SD | MET | A | 628 | 91.127 | 58.927 | 61.381 | 1.00 | 46.14 | 16 |
| ATOM | 2701 | CE | MET | A | 628 | 91.736 | 58.999 | 63.061 | 1.00 | 46.74 | 6 |
| ATOM | 2702 | C | MET | A | 628 | 95.004 | 58.330 | 58.874 | 1.00 | 25.76 | 6 |
| ATOM | 2703 | O | MET | A | 628 | 95.538 | 59.412 | 59.145 | 1.00 | 24.67 | 8 |
| ATOM | 2704 | N | GLN | A | 629 | 95.664 | 57.327 | 58.386 | 1.00 | 24.48 | 7 |
| ATOM | 2705 | CA | GLN | A | 629 | 97.083 | 57.507 | 58.141 | 1.00 | 25.53 | 6 |
| ATOM | 2706 | CB | GLN | A | 629 | 97.692 | 56.313 | 57.357 | 1.00 | 26.03 | 6 |
| ATOM | 2707 | CG | GLN | A | 629 | 97.510 | 54.916 | 57.925 | 1.00 | 30.99 | 6 |
| ATOM | 2708 | CD | GLN | A | 629 | 97.705 | 53.859 | 56.858 | 1.00 | 35.86 | 6 |
| ATOM | 2709 | OE1 | GLN | A | 629 | 98.796 | 53.680 | 56.320 | 1.00 | 34.53 | 8 |
| ATOM | 2710 | NE2 | GLN | A | 629 | 96.774 | 53.053 | 56.384 | 1.00 | 34.70 | 7 |
| ATOM | 2711 | C | GLN | A | 629 | 97.322 | 58.776 | 57.341 | 1.00 | 24.77 | 6 |
| ATOM | 2712 | O | GLN | A | 629 | 98.003 | 59.696 | 57.799 | 1.00 | 26.11 | 8 |
| ATOM | 2713 | N | LEU | A | 630 | 96.733 | 58.786 | 56.139 | 1.00 | 22.59 | 7 |
| ATOM | 2714 | CA | LEU | A | 630 | 96.879 | 59.899 | 55.225 | 1.00 | 23.66 | 6 |
| ATOM | 2715 | CB | LEU | A | 630 | 96.017 | 59.656 | 53.985 | 1.00 | 21.56 | 6 |
| ATOM | 2716 | CG | LEU | A | 630 | 96.326 | 58.355 | 53.263 | 1.00 | 25.59 | 6 |
| ATOM | 2717 | CD1 | LEU | A | 630 | 95.374 | 58.140 | 52.109 | 1.00 | 23.07 | 6 |
| ATOM | 2718 | CD2 | LEU | A | 630 | 97.768 | 58.353 | 52.791 | 1.00 | 24.62 | 6 |
| ATOM | 2719 | C | LEU | A | 630 | 96.601 | 61.248 | 55.843 | 1.00 | 24.21 | 6 |
| ATOM | 2720 | O | LEU | A | 630 | 96.854 | 62.264 | 55.199 | 1.00 | 23.22 | 8 |
| ATOM | 2721 | N | LEU | A | 631 | 96.076 | 61.290 | 57.060 | 1.00 | 22.93 | 7 |
| ATOM | 2722 | CA | LEU | A | 631 | 95.787 | 62.578 | 57.687 | 1.00 | 23.87 | 6 |
| ATOM | 2723 | CB | LEU | A | 631 | 94.309 | 62.643 | 58.115 | 1.00 | 18.83 | 6 |
| ATOM | 2724 | CG | LEU | A | 631 | 93.248 | 62.573 | 57.006 | 1.00 | 21.91 | 6 |
| ATOM | 2725 | CD1 | LEU | A | 631 | 91.868 | 62.423 | 57.621 | 1.00 | 23.94 | 6 |
| ATOM | 2726 | CD2 | LEU | A | 631 | 93.302 | 63.798 | 56.112 | 1.00 | 18.53 | 6 |
| ATOM | 2727 | C | LEU | A | 631 | 96.690 | 62.851 | 58.883 | 1.00 | 25.65 | 6 |
| ATOM | 2728 | O | LEU | A | 631 | 96.472 | 63.819 | 59.612 | 1.00 | 27.22 | 8 |
| ATOM | 2729 | N | ASP | A | 632 | 97.707 | 62.015 | 59.096 | 1.00 | 25.96 | 7 |
| ATOM | 2730 | CA | ASP | A | 632 | 98.595 | 62.251 | 60.226 | 1.00 | 28.42 | 6 |
| ATOM | 2731 | CB | ASP | A | 632 | 99.066 | 60.918 | 60.842 | 1.00 | 31.86 | 6 |
| ATOM | 2732 | CG | ASP | A | 632 | 100.191 | 60.254 | 60.063 | 1.00 | 37.69 | 6 |
| ATOM | 2733 | OD1 | ASP | A | 632 | 100.418 | 60.605 | 58.887 | 1.00 | 39.16 | 8 |
| ATOM | 2734 | OD2 | ASP | A | 632 | 100.844 | 59.356 | 60.640 | 1.00 | 37.54 | 8 |
| ATOM | 2735 | C | ASP | A | 632 | 99.776 | 63.150 | 59.848 | 1.00 | 29.40 | 6 |
| ATOM | 2736 | O | ASP | A | 632 | 99.800 | 63.723 | 58.753 | 1.00 | 27.08 | 8 |
| ATOM | 2737 | N | CYS | A | 633 | 100.745 | 63.272 | 60.753 | 1.00 | 27.95 | 7 |
| ATOM | 2738 | CA | CYS | A | 633 | 101.907 | 64.133 | 60.545 | 1.00 | 30.56 | 6 |
| ATOM | 2739 | CB | CYS | A | 633 | 102.750 | 64.188 | 61.827 | 1.00 | 32.03 | 6 |
| ATOM | 2740 | SG | CYS | A | 633 | 103.472 | 62.598 | 62.313 | 1.00 | 38.56 | 16 |
| ATOM | 2741 | C | CYS | A | 633 | 102.828 | 63.791 | 59.374 | 1.00 | 30.45 | 6 |
| ATOM | 2742 | O | CYS | A | 633 | 103.683 | 64.594 | 59.016 | 1.00 | 29.49 | 8 |
| ATOM | 2743 | N | ASN | A | 634 | 102.667 | 62.613 | 58.781 | 1.00 | 28.86 | 7 |
| ATOM | 2744 | CA | ASN | A | 634 | 103.566 | 62.195 | 57.679 | 1.00 | 29.86 | 6 |
| ATOM | 2745 | CB | ASN | A | 634 | 103.659 | 60.665 | 57.608 | 1.00 | 32.44 | 6 |
| ATOM | 2746 | CG | ASN | A | 634 | 103.618 | 60.047 | 58.979 | 1.00 | 34.47 | 6 |
| ATOM | 2747 | OD1 | ASN | A | 634 | 102.563 | 59.596 | 59.421 | 1.00 | 37.22 | 8 |
| ATOM | 2748 | ND2 | ASN | A | 634 | 104.767 | 60.013 | 59.665 | 1.00 | 38.81 | 7 |
| ATOM | 2749 | C | ASN | A | 634 | 103.164 | 62.708 | 56.308 | 1.00 | 28.53 | 6 |
| ATOM | 2750 | O | ASN | A | 634 | 103.992 | 62.851 | 55.410 | 1.00 | 29.23 | 8 |
| ATOM | 2751 | N | PHE | A | 635 | 101.883 | 62.966 | 56.163 | 1.00 | 29.34 | 7 |
| ATOM | 2752 | CA | PHE | A | 635 | 101.469 | 63.463 | 54.892 | 1.00 | 30.28 | 6 |
| ATOM | 2753 | CB | PHE | A | 635 | 100.291 | 62.766 | 54.335 | 1.00 | 31.95 | 6 |
| ATOM | 2754 | CG | PHE | A | 635 | 100.700 | 61.416 | 54.311 | 1.00 | 34.24 | 6 |
| ATOM | 2755 | CD1 | PHE | A | 635 | 100.438 | 60.669 | 55.421 | 1.00 | 34.24 | 6 |
| ATOM | 2756 | CD2 | PHE | A | 635 | 101.354 | 60.880 | 53.236 | 1.00 | 34.16 | 6 |
| ATOM | 2757 | CE1 | PHE | A | 635 | 100.828 | 59.350 | 55.463 | 1.00 | 34.73 | 6 |
| ATOM | 2758 | CE2 | PHE | A | 635 | 101.717 | 59.557 | 53.268 | 1.00 | 35.46 | 6 |
| ATOM | 2759 | CZ | PHE | A | 635 | 101.454 | 58.794 | 54.384 | 1.00 | 35.46 | 6 |
| ATOM | 2760 | C | PHE | A | 635 | 101.117 | 64.881 | 54.929 | 1.00 | 30.90 | 6 |
| ATOM | 2761 | O | PHE | A | 635 | 100.361 | 65.393 | 55.751 | 1.00 | 30.64 | 8 |
| ATOM | 2762 | N | SER | A | 636 | 101.738 | 65.546 | 53.998 | 1.00 | 29.14 | 7 |
| ATOM | 2763 | CA | SER | A | 636 | 101.376 | 66.874 | 53.712 | 1.00 | 30.07 | 6 |
| ATOM | 2764 | CB | SER | A | 636 | 102.526 | 67.850 | 53.578 | 1.00 | 26.76 | 6 |
| ATOM | 2765 | OG | SER | A | 636 | 103.633 | 67.261 | 52.928 | 1.00 | 30.07 | 8 |
| ATOM | 2766 | C | SER | A | 636 | 100.654 | 66.635 | 52.401 | 1.00 | 30.71 | 6 |
| ATOM | 2767 | O | SER | A | 636 | 99.757 | 65.788 | 52.334 | 1.00 | 33.36 | 8 |
| ATOM | 2768 | N | ASP | A | 637 | 101.016 | 67.323 | 51.353 | 1.00 | 30.06 | 7 |
| ATOM | 2769 | CA | ASP | A | 637 | 100.352 | 67.136 | 50.063 | 1.00 | 29.28 | 6 |
| ATOM | 2770 | CB | ASP | A | 637 | 100.856 | 65.941 | 49.312 | 1.00 | 27.34 | 6 |
| ATOM | 2771 | CG | ASP | A | 637 | 100.989 | 66.366 | 47.864 | 1.00 | 28.35 | 6 |
| ATOM | 2772 | OD1 | ASP | A | 637 | 99.951 | 66.590 | 47.209 | 1.00 | 27.54 | 8 |
| ATOM | 2773 | OD2 | ASP | A | 637 | 102.152 | 66.463 | 47.386 | 1.00 | 27.70 | 8 |
| ATOM | 2774 | C | ASP | A | 637 | 98.823 | 67.083 | 50.217 | 1.00 | 29.20 | 6 |
| ATOM | 2775 | O | ASP | A | 637 | 98.165 | 66.041 | 50.284 | 1.00 | 29.90 | 8 |
| ATOM | 2776 | N | GLU | A | 638 | 98.346 | 68.324 | 50.279 | 1.00 | 30.77 | 7 |
| ATOM | 2777 | CA | GLU | A | 638 | 96.980 | 68.739 | 50.385 | 1.00 | 33.60 | 6 |
| ATOM | 2778 | CB | GLU | A | 638 | 96.875 | 70.271 | 50.229 | 1.00 | 35.81 | 6 |
| ATOM | 2779 | CG | GLU | A | 638 | 97.778 | 71.034 | 51.184 | 1.00 | 43.69 | 6 |
| ATOM | 2780 | CD | GLU | A | 638 | 97.851 | 72.515 | 50.867 | 1.00 | 47.58 | 6 |
| ATOM | 2781 | OE1 | GLU | A | 638 | 97.336 | 72.921 | 49.804 | 1.00 | 50.12 | 8 |
| ATOM | 2782 | OE2 | GLU | A | 638 | 98.425 | 73.270 | 51.680 | 1.00 | 51.13 | 8 |
| ATOM | 2783 | C | GLU | A | 638 | 96.138 | 68.005 | 49.364 | 1.00 | 32.80 | 6 |
| ATOM | 2784 | O | GLU | A | 638 | 94.924 | 67.920 | 49.522 | 1.00 | 34.10 | 8 |
| ATOM | 2785 | N | ASN | A | 639 | 96.742 | 67.469 | 48.297 | 1.00 | 32.79 | 7 |
| ATOM | 2786 | CA | ASN | A | 639 | 95.998 | 66.652 | 47.336 | 1.00 | 31.08 | 6 |
| ATOM | 2787 | CB | ASN | A | 639 | 96.642 | 66.688 | 45.938 | 1.00 | 35.24 | 6 |
| ATOM | 2788 | CG | ASN | A | 639 | 97.508 | 67.916 | 45.738 | 1.00 | 39.09 | 6 |
| ATOM | 2789 | OD1 | ASN | A | 639 | 97.019 | 69.045 | 45.771 | 1.00 | 42.32 | 8 |
| ATOM | 2790 | ND2 | ASN | A | 639 | 98.804 | 67.706 | 45.549 | 1.00 | 43.18 | 7 |
| ATOM | 2791 | C | ASN | A | 639 | 95.945 | 65.243 | 47.879 | 1.00 | 29.13 | 6 |
| ATOM | 2792 | O | ASN | A | 639 | 94.958 | 64.532 | 47.715 | 1.00 | 28.74 | 8 |
| ATOM | 2793 | N | VAL | A | 640 | 97.025 | 64.816 | 48.537 | 1.00 | 28.91 | 7 |
| ATOM | 2794 | CA | VAL | A | 640 | 97.087 | 63.502 | 49.150 | 1.00 | 26.24 | 6 |
| ATOM | 2795 | CB | VAL | A | 640 | 98.510 | 63.229 | 49.700 | 1.00 | 28.62 | 6 |
| ATOM | 2796 | CG1 | VAL | A | 640 | 98.523 | 61.999 | 50.598 | 1.00 | 25.93 | 6 |
| ATOM | 2797 | CG2 | VAL | A | 640 | 99.481 | 63.046 | 48.548 | 1.00 | 26.18 | 6 |
| ATOM | 2798 | C | VAL | A | 640 | 96.091 | 63.390 | 50.276 | 1.00 | 27.41 | 6 |
| ATOM | 2799 | O | VAL | A | 640 | 95.337 | 62.424 | 50.387 | 1.00 | 27.12 | 8 |
| ATOM | 2800 | N | ARG | A | 641 | 96.121 | 64.406 | 51.109 | 1.00 | 25.14 | 7 |
| ATOM | 2801 | CA | ARG | A | 641 | 95.253 | 64.470 | 52.258 | 1.00 | 25.26 | 6 |
| ATOM | 2802 | CB | ARG | A | 641 | 95.719 | 65.606 | 53.179 | 1.00 | 24.56 | 6 |
| ATOM | 2803 | CG | ARG | A | 641 | 94.646 | 66.174 | 54.082 | 1.00 | 21.86 | 6 |
| ATOM | 2804 | CD | ARG | A | 641 | 95.221 | 66.949 | 55.247 | 1.00 | 22.05 | 6 |
| ATOM | 2805 | NE | ARG | A | 641 | 96.468 | 66.362 | 55.736 | 1.00 | 20.96 | 7 |
| ATOM | 2806 | CZ | ARG | A | 641 | 96.790 | 66.180 | 57.038 | 1.00 | 22.89 | 6 |
| ATOM | 2807 | NH1 | ARG | A | 641 | 95.938 | 66.565 | 57.989 | 1.00 | 17.75 | 7 |
| ATOM | 2808 | NH2 | ARG | A | 641 | 97.938 | 65.622 | 57.369 | 1.00 | 20.93 | 7 |
| ATOM | 2809 | C | ARG | A | 641 | 93.772 | 64.654 | 51.893 | 1.00 | 25.39 | 6 |
| ATOM | 2810 | O | ARG | A | 641 | 92.878 | 64.162 | 52.604 | 1.00 | 26.76 | 8 |
| ATOM | 2811 | N | ALA | A | 642 | 93.514 | 65.371 | 50.794 | 1.00 | 26.97 | 7 |
| ATOM | 2812 | CA | ALA | A | 642 | 92.172 | 65.689 | 50.332 | 1.00 | 28.07 | 6 |
| ATOM | 2813 | CB | ALA | A | 642 | 92.214 | 66.757 | 49.238 | 1.00 | 30.04 | 6 |
| ATOM | 2814 | C | ALA | A | 642 | 91.478 | 64.460 | 49.808 | 1.00 | 27.10 | 6 |
| ATOM | 2815 | O | ALA | A | 642 | 90.295 | 64.518 | 49.501 | 1.00 | 27.04 | 8 |
| ATOM | 2816 | N | ILE | A | 643 | 92.148 | 63.345 | 49.699 | 1.00 | 28.59 | 7 |
| ATOM | 2817 | CA | ILE | A | 643 | 91.420 | 62.245 | 49.169 | 1.00 | 28.32 | 6 |
| ATOM | 2818 | CB | ILE | A | 643 | 92.109 | 61.751 | 47.896 | 1.00 | 31.87 | 6 |
| ATOM | 2819 | CG2 | ILE | A | 643 | 92.215 | 62.884 | 46.884 | 1.00 | 32.42 | 6 |
| ATOM | 2820 | CG1 | ILE | A | 643 | 93.492 | 61.182 | 48.201 | 1.00 | 31.17 | 6 |
| ATOM | 2821 | CD1 | ILE | A | 643 | 94.266 | 60.761 | 46.970 | 1.00 | 36.35 | 6 |
| ATOM | 2822 | C | ILE | A | 643 | 91.186 | 61.229 | 50.268 | 1.00 | 27.87 | 6 |
| ATOM | 2823 | O | ILE | A | 643 | 90.420 | 60.268 | 50.105 | 1.00 | 27.38 | 8 |
| ATOM | 2824 | N | ALA | A | 644 | 91.872 | 61.447 | 51.400 | 1.00 | 26.59 | 7 |
| ATOM | 2825 | CA | ALA | A | 644 | 91.603 | 60.682 | 52.586 | 1.00 | 25.99 | 6 |
| ATOM | 2826 | CB | ALA | A | 644 | 92.615 | 60.900 | 53.707 | 1.00 | 26.96 | 6 |
| ATOM | 2827 | C | ALA | A | 644 | 90.217 | 61.224 | 52.970 | 1.00 | 24.66 | 6 |
| ATOM | 2828 | O | ALA | A | 644 | 89.295 | 60.470 | 53.298 | 1.00 | 24.86 | 8 |
| ATOM | 2829 | N | VAL | A | 645 | 90.079 | 62.544 | 52.906 | 1.00 | 25.43 | 7 |
| ATOM | 2830 | CA | VAL | A | 645 | 88.819 | 63.212 | 53.211 | 1.00 | 27.33 | 6 |
| ATOM | 2831 | CB | VAL | A | 645 | 88.971 | 64.744 | 53.123 | 1.00 | 25.65 | 6 |
| ATOM | 2832 | CG1 | VAL | A | 645 | 87.611 | 65.416 | 53.217 | 1.00 | 27.03 | 6 |
| ATOM | 2833 | CG2 | VAL | A | 645 | 89.895 | 65.263 | 54.217 | 1.00 | 24.42 | 6 |
| ATOM | 2834 | C | VAL | A | 645 | 87.698 | 62.760 | 52.278 | 1.00 | 28.88 | 6 |
| ATOM | 2835 | O | VAL | A | 645 | 86.603 | 62.462 | 52.761 | 1.00 | 27.58 | 8 |
| ATOM | 2836 | N | GLN | A | 646 | 87.906 | 62.709 | 50.976 | 1.00 | 30.84 | 7 |
| ATOM | 2837 | CA | GLN | A | 646 | 86.794 | 62.283 | 50.097 | 1.00 | 32.04 | 6 |
| ATOM | 2838 | CB | GLN | A | 646 | 87.283 | 62.229 | 48.641 | 1.00 | 35.35 | 6 |
| ATOM | 2839 | CG | GLN | A | 646 | 86.403 | 61.425 | 47.699 | 1.00 | 41.08 | 6 |
| ATOM | 2840 | CD | GLN | A | 646 | 87.072 | 61.170 | 46.371 | 1.00 | 44.00 | 6 |
| ATOM | 2841 | OE1 | GLN | A | 646 | 86.731 | 60.209 | 45.691 | 1.00 | 45.31 | 8 |
| ATOM | 2842 | NE2 | GLN | A | 646 | 88.034 | 61.893 | 45.802 | 1.00 | 44.33 | 7 |
| ATOM | 2843 | C | GLN | A | 646 | 86.214 | 60.922 | 50.509 | 1.00 | 32.19 | 6 |
| ATOM | 2844 | O | GLN | A | 646 | 85.045 | 60.597 | 50.297 | 1.00 | 33.01 | 8 |
| ATOM | 2845 | N | LYS | A | 647 | 87.110 | 60.149 | 51.130 | 1.00 | 30.13 | 7 |
| ATOM | 2846 | CA | LYS | A | 647 | 86.897 | 58.814 | 51.658 | 1.00 | 31.36 | 6 |
| ATOM | 2847 | CB | LYS | A | 647 | 88.206 | 58.299 | 52.286 | 1.00 | 32.66 | 6 |
| ATOM | 2848 | CG | LYS | A | 647 | 88.713 | 56.998 | 51.749 | 1.00 | 39.31 | 6 |
| ATOM | 2849 | CD | LYS | A | 647 | 89.077 | 57.152 | 50.288 | 1.00 | 41.07 | 6 |
| ATOM | 2850 | CE | LYS | A | 647 | 88.805 | 55.868 | 49.504 | 1.00 | 43.21 | 6 |
| ATOM | 2851 | NZ | LYS | A | 647 | 89.529 | 54.698 | 50.081 | 1.00 | 44.13 | 7 |
| ATOM | 2852 | C | LYS | A | 647 | 85.891 | 58.796 | 52.747 | 1.00 | 31.09 | 6 |
| ATOM | 2853 | O | LYS | A | 647 | 84.910 | 58.059 | 52.783 | 1.00 | 32.34 | 8 |
| ATOM | 2854 | N | LEU | A | 648 | 86.253 | 59.668 | 53.643 | 1.00 | 28.97 | 7 |
| ATOM | 2855 | CA | LEU | A | 648 | 85.526 | 59.898 | 54.839 | 1.00 | 27.95 | 6 |
| ATOM | 2856 | CB | LEU | A | 648 | 86.345 | 60.842 | 55.724 | 1.00 | 25.96 | 6 |
| ATOM | 2857 | CG | LEU | A | 648 | 87.722 | 60.272 | 56.086 | 1.00 | 26.50 | 6 |
| ATOM | 2858 | CD1 | LEU | A | 648 | 88.592 | 61.339 | 56.730 | 1.00 | 26.87 | 6 |
| ATOM | 2859 | CD2 | LEU | A | 648 | 87.578 | 59.075 | 57.017 | 1.00 | 27.55 | 6 |
| ATOM | 2860 | C | LEU | A | 648 | 84.094 | 60.379 | 54.553 | 1.00 | 27.19 | 6 |
| ATOM | 2861 | O | LEU | A | 648 | 83.193 | 60.147 | 55.368 | 1.00 | 28.32 | 8 |
| ATOM | 2862 | N | GLU | A | 649 | 83.882 | 61.059 | 53.422 | 1.00 | 28.97 | 7 |
| ATOM | 2863 | CA | GLU | A | 649 | 82.539 | 61.568 | 53.006 | 1.00 | 31.88 | 6 |
| ATOM | 2864 | CB | GLU | A | 649 | 82.628 | 62.192 | 51.605 | 1.00 | 33.27 | 6 |
| ATOM | 2865 | CG | GLU | A | 649 | 83.505 | 63.451 | 51.527 | 1.00 | 36.44 | 6 |
| ATOM | 2866 | CD | GLU | A | 649 | 82.815 | 64.701 | 52.051 | 1.00 | 37.45 | 6 |
| ATOM | 2867 | OE1 | GLU | A | 649 | 81.690 | 64.585 | 52.582 | 1.00 | 38.09 | 8 |
| ATOM | 2868 | OE2 | GLU | A | 649 | 83.395 | 65.812 | 51.933 | 1.00 | 39.56 | 8 |
| ATOM | 2869 | C | GLU | A | 649 | 81.457 | 60.484 | 53.030 | 1.00 | 32.19 | 6 |
| ATOM | 2870 | O | GLU | A | 649 | 80.276 | 60.789 | 53.176 | 1.00 | 32.73 | 8 |
| ATOM | 2871 | N | SER | A | 650 | 81.852 | 59.222 | 52.873 | 1.00 | 31.70 | 7 |
| ATOM | 2872 | CA | SER | A | 650 | 80.881 | 58.136 | 52.862 | 1.00 | 33.40 | 6 |
| ATOM | 2873 | CB | SER | A | 650 | 81.363 | 57.006 | 51.948 | 1.00 | 34.84 | 6 |
| ATOM | 2874 | OG | SER | A | 650 | 82.337 | 56.217 | 52.605 | 1.00 | 35.88 | 8 |
| ATOM | 2875 | C | SER | A | 650 | 80.543 | 57.551 | 54.236 | 1.00 | 33.28 | 6 |
| ATOM | 2876 | O | SER | A | 650 | 79.770 | 56.605 | 54.328 | 1.00 | 33.85 | 8 |
| ATOM | 2877 | N | LEU | A | 651 | 81.124 | 58.095 | 55.302 | 1.00 | 32.18 | 7 |
| ATOM | 2878 | CA | LEU | A | 651 | 80.828 | 57.590 | 56.639 | 1.00 | 31.39 | 6 |
| ATOM | 2879 | CB | LEU | A | 651 | 81.821 | 58.149 | 57.662 | 1.00 | 32.40 | 6 |
| ATOM | 2880 | CG | LEU | A | 651 | 83.256 | 57.629 | 57.682 | 1.00 | 34.49 | 6 |
| ATOM | 2881 | CD1 | LEU | A | 651 | 84.109 | 58.559 | 58.527 | 1.00 | 33.00 | 6 |
| ATOM | 2882 | CD2 | LEU | A | 651 | 83.286 | 56.221 | 58.230 | 1.00 | 32.88 | 6 |
| ATOM | 2883 | C | LEU | A | 651 | 79.427 | 58.028 | 57.033 | 1.00 | 30.88 | 6 |
| ATOM | 2884 | O | LEU | A | 651 | 79.014 | 59.149 | 56.721 | 1.00 | 29.87 | 8 |
| ATOM | 2885 | N | GLU | A | 652 | 78.677 | 57.152 | 57.693 | 1.00 | 30.09 | 7 |
| ATOM | 2886 | CA | GLU | A | 652 | 77.348 | 57.558 | 58.123 | 1.00 | 31.63 | 6 |
| ATOM | 2887 | CB | GLU | A | 652 | 76.410 | 56.354 | 58.263 | 1.00 | 34.09 | 6 |
| ATOM | 2888 | CG | GLU | A | 652 | 76.765 | 55.333 | 59.337 | 1.00 | 36.31 | 6 |
| ATOM | 2889 | CD | GLU | A | 652 | 75.753 | 54.188 | 59.376 | 1.00 | 39.22 | 6 |
| ATOM | 2890 | OE1 | GLU | A | 652 | 75.802 | 53.358 | 60.308 | 1.00 | 36.29 | 8 |
| ATOM | 2891 | OE2 | GLU | A | 652 | 74.905 | 54.122 | 58.455 | 1.00 | 42.27 | 8 |
| ATOM | 2892 | C | GLU | A | 652 | 77.551 | 58.268 | 59.459 | 1.00 | 31.80 | 6 |
| ATOM | 2893 | O | GLU | A | 652 | 78.568 | 58.066 | 60.115 | 1.00 | 31.04 | 8 |
| ATOM | 2894 | N | ASP | A | 653 | 76.601 | 59.112 | 59.850 | 1.00 | 32.26 | 7 |
| ATOM | 2895 | CA | ASP | A | 653 | 76.730 | 59.864 | 61.094 | 1.00 | 31.46 | 6 |
| ATOM | 2896 | CB | ASP | A | 653 | 75.410 | 60.529 | 61.458 | 1.00 | 29.85 | 6 |
| ATOM | 2897 | CG | ASP | A | 653 | 75.035 | 61.636 | 60.512 | 1.00 | 30.95 | 6 |
| ATOM | 2898 | OD1 | ASP | A | 653 | 75.931 | 62.244 | 59.883 | 1.00 | 30.50 | 8 |
| ATOM | 2899 | OD2 | ASP | A | 653 | 73.825 | 61.919 | 60.422 | 1.00 | 32.47 | 8 |
| ATOM | 2900 | C | ASP | A | 653 | 77.213 | 59.056 | 62.290 | 1.00 | 30.77 | 6 |
| ATOM | 2901 | O | ASP | A | 653 | 78.063 | 59.522 | 63.060 | 1.00 | 29.88 | 8 |
| ATOM | 2902 | N | ASP | A | 654 | 76.681 | 57.851 | 62.462 | 1.00 | 30.30 | 7 |
| ATOM | 2903 | CA | ASP | A | 654 | 77.099 | 57.041 | 63.600 | 1.00 | 31.22 | 6 |
| ATOM | 2904 | CB | ASP | A | 654 | 76.340 | 55.716 | 63.667 | 1.00 | 33.98 | 6 |
| ATOM | 2905 | CG | ASP | A | 654 | 76.957 | 54.746 | 64.676 | 1.00 | 38.76 | 6 |
| ATOM | 2906 | OD1 | ASP | A | 654 | 76.974 | 55.072 | 65.886 | 1.00 | 38.21 | 8 |
| ATOM | 2907 | OD2 | ASP | A | 654 | 77.427 | 53.663 | 64.261 | 1.00 | 39.42 | 8 |
| ATOM | 2908 | C | ASP | A | 654 | 78.587 | 56.752 | 63.579 | 1.00 | 29.90 | 6 |
| ATOM | 2909 | O | ASP | A | 654 | 79.215 | 56.669 | 64.636 | 1.00 | 30.74 | 8 |
| ATOM | 2910 | N | ASP | A | 655 | 79.159 | 56.589 | 62.391 | 1.00 | 26.96 | 7 |
| ATOM | 2911 | CA | ASP | A | 655 | 80.589 | 56.312 | 62.312 | 1.00 | 27.98 | 6 |
| ATOM | 2912 | CB | ASP | A | 655 | 80.916 | 55.608 | 61.000 | 1.00 | 27.70 | 6 |
| ATOM | 2913 | CG | ASP | A | 655 | 80.631 | 54.119 | 61.046 | 1.00 | 34.13 | 6 |
| ATOM | 2914 | OD1 | ASP | A | 655 | 80.964 | 53.487 | 62.079 | 1.00 | 35.58 | 8 |
| ATOM | 2915 | OD2 | ASP | A | 655 | 80.090 | 53.580 | 60.058 | 1.00 | 37.41 | 8 |
| ATOM | 2916 | C | ASP | A | 655 | 81.427 | 57.569 | 62.500 | 1.00 | 23.22 | 6 |
| ATOM | 2917 | O | ASP | A | 655 | 82.499 | 57.512 | 63.091 | 1.00 | 24.27 | 8 |
| ATOM | 2918 | N | VAL | A | 656 | 80.933 | 58.696 | 62.009 | 1.00 | 22.37 | 7 |
| ATOM | 2919 | CA | VAL | A | 656 | 81.643 | 59.952 | 62.205 | 1.00 | 22.52 | 6 |
| ATOM | 2920 | CB | VAL | A | 656 | 80.913 | 61.134 | 61.470 | 1.00 | 23.31 | 6 |
| ATOM | 2921 | CG1 | VAL | A | 656 | 81.421 | 62.474 | 61.993 | 1.00 | 21.57 | 6 |
| ATOM | 2922 | CG2 | VAL | A | 656 | 81.110 | 61.034 | 59.953 | 1.00 | 22.09 | 6 |
| ATOM | 2923 | C | VAL | A | 656 | 81.813 | 60.243 | 63.734 | 1.00 | 23.54 | 6 |
| ATOM | 2924 | O | VAL | A | 656 | 82.875 | 60.710 | 64.149 | 1.00 | 24.13 | 8 |
| ATOM | 2925 | N | LEU | A | 657 | 80.795 | 59.995 | 64.540 | 1.00 | 25.78 | 7 |
| ATOM | 2926 | CA | LEU | A | 657 | 80.894 | 60.249 | 65.982 | 1.00 | 25.09 | 6 |
| ATOM | 2927 | CB | LEU | A | 657 | 79.530 | 60.096 | 66.637 | 1.00 | 25.44 | 6 |
| ATOM | 2928 | CG | LEU | A | 657 | 78.673 | 61.352 | 66.789 | 1.00 | 25.86 | 6 |
| ATOM | 2929 | CD1 | LEU | A | 657 | 77.308 | 61.010 | 67.361 | 1.00 | 24.25 | 6 |
| ATOM | 2930 | CD2 | LEU | A | 657 | 79.381 | 62.385 | 67.658 | 1.00 | 24.53 | 6 |
| ATOM | 2931 | C | LEU | A | 657 | 81.841 | 59.321 | 66.673 | 1.00 | 26.32 | 6 |
| ATOM | 2932 | O | LEU | A | 657 | 82.149 | 59.497 | 67.865 | 1.00 | 25.53 | 8 |
| ATOM | 2933 | N | HIS | A | 658 | 82.319 | 58.326 | 65.972 | 1.00 | 26.32 | 7 |
| ATOM | 2934 | CA | HIS | A | 658 | 83.261 | 57.403 | 66.565 | 1.00 | 25.83 | 6 |
| ATOM | 2935 | CB | HIS | A | 658 | 83.123 | 56.030 | 65.956 | 1.00 | 30.78 | 6 |
| ATOM | 2936 | CG | HIS | A | 658 | 81.789 | 55.446 | 66.341 | 1.00 | 36.07 | 6 |
| ATOM | 2937 | CD2 | HIS | A | 658 | 81.427 | 54.696 | 67.407 | 1.00 | 36.62 | 6 |
| ATOM | 2938 | ND1 | HIS | A | 658 | 80.652 | 55.603 | 65.578 | 1.00 | 41.49 | 7 |
| ATOM | 2939 | CE1 | HIS | A | 658 | 79.645 | 54.976 | 66.162 | 1.00 | 40.53 | 6 |
| ATOM | 2940 | NE2 | HIS | A | 658 | 80.089 | 54.419 | 67.274 | 1.00 | 40.67 | 7 |
| ATOM | 2941 | C | HIS | A | 658 | 84.640 | 57.842 | 66.346 | 1.00 | 24.83 | 6 |
| ATOM | 2942 | O | HIS | A | 658 | 85.551 | 57.510 | 67.095 | 1.00 | 24.83 | 8 |
| ATOM | 2943 | N | TYR | A | 659 | 84.801 | 58.583 | 65.322 | 1.00 | 22.48 | 7 |
| ATOM | 2944 | CA | TYR | A | 659 | 86.099 | 59.040 | 65.029 | 1.00 | 22.72 | 6 |
| ATOM | 2945 | CB | TYR | A | 659 | 86.502 | 58.562 | 63.642 | 1.00 | 22.56 | 6 |
| ATOM | 2946 | CG | TYR | A | 659 | 86.517 | 57.070 | 63.520 | 1.00 | 27.07 | 6 |
| ATOM | 2947 | CD1 | TYR | A | 659 | 85.519 | 56.356 | 62.861 | 1.00 | 26.24 | 6 |
| ATOM | 2948 | CE1 | TYR | A | 659 | 85.558 | 54.960 | 62.774 | 1.00 | 31.15 | 6 |
| ATOM | 2949 | CD2 | TYR | A | 659 | 87.554 | 56.357 | 64.080 | 1.00 | 27.01 | 6 |
| ATOM | 2950 | CE2 | TYR | A | 659 | 87.599 | 54.974 | 64.031 | 1.00 | 30.22 | 6 |
| ATOM | 2951 | CZ | TYR | A | 659 | 86.606 | 54.282 | 63.384 | 1.00 | 31.42 | 6 |
| ATOM | 2952 | OH | TYR | A | 659 | 86.636 | 52.907 | 63.332 | 1.00 | 37.03 | 8 |
| ATOM | 2953 | C | TYR | A | 659 | 86.198 | 60.546 | 65.122 | 1.00 | 21.59 | 6 |
| ATOM | 2954 | O | TYR | A | 659 | 87.224 | 61.125 | 64.759 | 1.00 | 22.01 | 8 |
| ATOM | 2955 | N | LEU | A | 660 | 85.167 | 61.196 | 65.614 | 1.00 | 20.37 | 7 |
| ATOM | 2956 | CA | LEU | A | 660 | 85.309 | 62.641 | 65.672 | 1.00 | 20.61 | 6 |
| ATOM | 2957 | CB | LEU | A | 660 | 84.136 | 63.317 | 66.359 | 1.00 | 19.19 | 6 |
| ATOM | 2958 | CG | LEU | A | 660 | 84.078 | 64.840 | 66.118 | 1.00 | 21.13 | 6 |
| ATOM | 2959 | CD1 | LEU | A | 660 | 84.151 | 65.145 | 64.629 | 1.00 | 19.95 | 6 |
| ATOM | 2960 | CD2 | LEU | A | 660 | 82.824 | 65.440 | 66.727 | 1.00 | 19.56 | 6 |
| ATOM | 2961 | C | LEU | A | 660 | 86.625 | 63.043 | 66.347 | 1.00 | 19.85 | 6 |
| ATOM | 2962 | O | LEU | A | 660 | 87.541 | 63.487 | 65.650 | 1.00 | 22.45 | 8 |
| ATOM | 2963 | N | LEU | A | 661 | 86.747 | 62.910 | 67.699 | 1.00 | 19.28 | 7 |
| ATOM | 2964 | CA | LEU | A | 661 | 87.956 | 63.350 | 68.458 | 1.00 | 19.95 | 6 |
| ATOM | 2965 | CB | LEU | A | 661 | 88.142 | 62.552 | 69.772 | 1.00 | 19.00 | 6 |
| ATOM | 2966 | CG | LEU | A | 661 | 89.145 | 63.107 | 70.805 | 1.00 | 20.12 | 6 |
| ATOM | 2967 | CD1 | LEU | A | 661 | 88.904 | 64.589 | 71.060 | 1.00 | 16.41 | 6 |
| ATOM | 2968 | CD2 | LEU | A | 661 | 89.066 | 62.321 | 72.109 | 1.00 | 18.83 | 6 |
| ATOM | 2969 | C | LEU | A | 661 | 89.295 | 63.308 | 67.657 | 1.00 | 20.90 | 6 |
| ATOM | 2970 | O | LEU | A | 661 | 89.953 | 64.344 | 67.562 | 1.00 | 19.64 | 8 |
| ATOM | 2971 | N | GLN | A | 662 | 89.728 | 62.188 | 67.079 | 1.00 | 20.78 | 7 |
| ATOM | 2972 | CA | GLN | A | 662 | 90.994 | 62.233 | 66.315 | 1.00 | 20.85 | 6 |
| ATOM | 2973 | CB | GLN | A | 662 | 91.432 | 60.839 | 65.828 | 1.00 | 24.65 | 6 |
| ATOM | 2974 | CG | GLN | A | 662 | 91.848 | 59.853 | 66.901 | 1.00 | 32.64 | 6 |
| ATOM | 2975 | CD | GLN | A | 662 | 90.698 | 58.958 | 67.237 | 1.00 | 33.81 | 6 |
| ATOM | 2976 | OE1 | GLN | A | 662 | 89.647 | 59.428 | 67.682 | 1.00 | 36.40 | 8 |
| ATOM | 2977 | NE2 | GLN | A | 662 | 90.659 | 57.646 | 67.100 | 1.00 | 37.45 | 7 |
| ATOM | 2978 | C | GLN | A | 662 | 90.801 | 63.124 | 65.092 | 1.00 | 21.61 | 6 |
| ATOM | 2979 | O | GLN | A | 662 | 91.646 | 63.957 | 64.784 | 1.00 | 21.18 | 8 |
| ATOM | 2980 | N | LEU | A | 663 | 89.676 | 62.932 | 64.380 | 1.00 | 20.11 | 7 |
| ATOM | 2981 | CA | LEU | A | 663 | 89.412 | 63.761 | 63.213 | 1.00 | 19.06 | 6 |
| ATOM | 2982 | CB | LEU | A | 663 | 88.016 | 63.506 | 62.675 | 1.00 | 18.31 | 6 |
| ATOM | 2983 | CG | LEU | A | 663 | 87.840 | 62.211 | 61.880 | 1.00 | 21.54 | 6 |
| ATOM | 2984 | CD1 | LEU | A | 663 | 86.477 | 62.189 | 61.209 | 1.00 | 18.85 | 6 |
| ATOM | 2985 | CD2 | LEU | A | 663 | 88.947 | 62.053 | 60.864 | 1.00 | 24.05 | 6 |
| ATOM | 2986 | C | LEU | A | 663 | 89.668 | 65.234 | 63.603 | 1.00 | 20.38 | 6 |
| ATOM | 2987 | O | LEU | A | 663 | 90.396 | 65.950 | 62.915 | 1.00 | 20.16 | 8 |
| ATOM | 2988 | N | VAL | A | 664 | 89.083 | 65.679 | 64.710 | 1.00 | 18.99 | 7 |
| ATOM | 2989 | CA | VAL | A | 664 | 89.319 | 67.043 | 65.180 | 1.00 | 18.29 | 6 |
| ATOM | 2990 | CB | VAL | A | 664 | 88.461 | 67.367 | 66.427 | 1.00 | 19.25 | 6 |
| ATOM | 2991 | CG1 | VAL | A | 664 | 88.778 | 68.763 | 66.959 | 1.00 | 20.97 | 6 |
| ATOM | 2992 | CG2 | VAL | A | 664 | 86.966 | 67.256 | 66.114 | 1.00 | 20.75 | 6 |
| ATOM | 2993 | C | VAL | A | 664 | 90.797 | 67.203 | 65.537 | 1.00 | 19.43 | 6 |
| ATOM | 2994 | O | VAL | A | 664 | 91.427 | 68.198 | 65.183 | 1.00 | 19.07 | 8 |
| ATOM | 2995 | N | GLN | A | 665 | 91.369 | 66.225 | 66.236 | 1.00 | 19.27 | 7 |
| ATOM | 2996 | CA | GLN | A | 665 | 92.789 | 66.330 | 66.579 | 1.00 | 19.89 | 6 |
| ATOM | 2997 | CB | GLN | A | 665 | 93.221 | 65.154 | 67.467 | 1.00 | 21.40 | 6 |
| ATOM | 2998 | CG | GLN | A | 665 | 93.043 | 65.386 | 68.976 | 1.00 | 22.65 | 6 |
| ATOM | 2999 | CD | GLN | A | 665 | 93.872 | 66.563 | 69.489 | 1.00 | 28.77 | 6 |
| ATOM | 3000 | OE1 | GLN | A | 665 | 95.065 | 66.663 | 69.203 | 1.00 | 32.49 | 8 |
| ATOM | 3001 | NE2 | GLN | A | 665 | 93.428 | 67.550 | 70.248 | 1.00 | 29.04 | 7 |
| ATOM | 3002 | C | GLN | A | 665 | 93.676 | 66.373 | 65.329 | 1.00 | 19.94 | 6 |
| ATOM | 3003 | O | GLN | A | 665 | 94.696 | 67.072 | 65.295 | 1.00 | 16.63 | 8 |
| ATOM | 3004 | N | ALA | A | 666 | 93.276 | 65.630 | 64.300 | 1.00 | 20.46 | 7 |
| ATOM | 3005 | CA | ALA | A | 666 | 94.048 | 65.557 | 63.064 | 1.00 | 20.53 | 6 |
| ATOM | 3006 | CB | ALA | A | 666 | 93.480 | 64.484 | 62.164 | 1.00 | 16.77 | 6 |
| ATOM | 3007 | C | ALA | A | 666 | 94.104 | 66.905 | 62.327 | 1.00 | 19.79 | 6 |
| ATOM | 3008 | O | ALA | A | 666 | 94.919 | 67.064 | 61.419 | 1.00 | 19.71 | 8 |
| ATOM | 3009 | N | VAL | A | 667 | 93.253 | 67.848 | 62.686 | 1.00 | 19.58 | 7 |
| ATOM | 3010 | CA | VAL | A | 667 | 93.299 | 69.163 | 62.055 | 1.00 | 19.41 | 6 |
| ATOM | 3011 | CB | VAL | A | 667 | 92.147 | 70.074 | 62.587 | 1.00 | 21.46 | 6 |
| ATOM | 3012 | CG1 | VAL | A | 667 | 92.389 | 71.511 | 62.171 | 1.00 | 22.17 | 6 |
| ATOM | 3013 | CG2 | VAL | A | 667 | 90.787 | 69.593 | 62.093 | 1.00 | 22.12 | 6 |
| ATOM | 3014 | C | VAL | A | 667 | 94.660 | 69.775 | 62.324 | 1.00 | 20.78 | 6 |
| ATOM | 3015 | O | VAL | A | 667 | 95.136 | 70.625 | 61.572 | 1.00 | 20.17 | 8 |
| ATOM | 3016 | N | LYS | A | 668 | 95.286 | 69.338 | 63.376 | 1.00 | 20.03 | 7 |
| ATOM | 3017 | CA | LYS | A | 668 | 96.610 | 69.855 | 63.700 | 1.00 | 19.79 | 6 |
| ATOM | 3018 | CB | LYS | A | 668 | 97.110 | 69.301 | 65.041 | 1.00 | 20.06 | 6 |
| ATOM | 3019 | CG | LYS | A | 668 | 96.283 | 69.761 | 66.223 | 1.00 | 23.77 | 6 |
| ATOM | 3020 | CD | LYS | A | 668 | 96.841 | 69.188 | 67.521 | 1.00 | 21.56 | 6 |
| ATOM | 3021 | CE | LYS | A | 668 | 96.071 | 69.689 | 68.729 | 1.00 | 23.99 | 6 |
| ATOM | 3022 | NZ | LYS | A | 668 | 96.599 | 69.125 | 69.999 | 1.00 | 24.85 | 7 |
| ATOM | 3023 | C | LYS | A | 668 | 97.605 | 69.549 | 62.624 | 1.00 | 19.92 | 6 |
| ATOM | 3024 | O | LYS | A | 668 | 98.441 | 70.386 | 62.256 | 1.00 | 18.46 | 8 |
| ATOM | 3025 | N | PHE | A | 669 | 97.496 | 68.356 | 62.131 | 1.00 | 17.67 | 7 |
| ATOM | 3026 | CA | PHE | A | 669 | 98.432 | 67.987 | 61.118 | 1.00 | 19.12 | 6 |
| ATOM | 3027 | CB | PHE | A | 669 | 98.368 | 66.468 | 60.932 | 1.00 | 21.70 | 6 |
| ATOM | 3028 | CG | PHE | A | 669 | 98.971 | 65.704 | 62.072 | 1.00 | 18.18 | 6 |
| ATOM | 3029 | CD1 | PHE | A | 669 | 98.215 | 64.743 | 62.718 | 1.00 | 20.74 | 6 |
| ATOM | 3030 | CD2 | PHE | A | 669 | 100.279 | 65.920 | 62.493 | 1.00 | 21.98 | 6 |
| ATOM | 3031 | CE1 | PHE | A | 669 | 98.745 | 64.006 | 63.778 | 1.00 | 23.55 | 6 |
| ATOM | 3032 | CE2 | PHE | A | 669 | 100.817 | 65.191 | 63.548 | 1.00 | 22.52 | 6 |
| ATOM | 3033 | CZ | PHE | A | 669 | 100.047 | 64.232 | 64.190 | 1.00 | 21.70 | 6 |
| ATOM | 3034 | C | PHE | A | 669 | 98.250 | 68.786 | 59.829 | 1.00 | 22.99 | 6 |
| ATOM | 3035 | O | PHE | A | 669 | 99.057 | 68.618 | 58.922 | 1.00 | 23.48 | 8 |
| ATOM | 3036 | N | GLU | A | 670 | 97.221 | 69.651 | 59.717 | 1.00 | 21.13 | 7 |
| ATOM | 3037 | CA | GLU | A | 670 | 96.994 | 70.481 | 58.523 | 1.00 | 22.57 | 6 |
| ATOM | 3038 | CB | GLU | A | 670 | 95.522 | 70.913 | 58.471 | 1.00 | 23.59 | 6 |
| ATOM | 3039 | CG | GLU | A | 670 | 94.604 | 70.102 | 57.583 | 1.00 | 27.36 | 6 |
| ATOM | 3040 | CD | GLU | A | 670 | 95.359 | 69.091 | 56.732 | 1.00 | 29.50 | 6 |
| ATOM | 3041 | OE1 | GLU | A | 670 | 95.582 | 69.366 | 55.535 | 1.00 | 30.49 | 8 |
| ATOM | 3042 | OE2 | GLU | A | 670 | 95.734 | 68.023 | 57.268 | 1.00 | 30.23 | 8 |
| ATOM | 3043 | C | GLU | A | 670 | 97.841 | 71.760 | 58.529 | 1.00 | 23.57 | 6 |
| ATOM | 3044 | O | GLU | A | 670 | 97.997 | 72.378 | 59.592 | 1.00 | 19.88 | 8 |
| ATOM | 3045 | N | PRO | A | 671 | 98.428 | 72.197 | 57.352 | 1.00 | 24.42 | 7 |
| ATOM | 3046 | CD | PRO | A | 671 | 99.296 | 71.297 | 56.597 | 1.00 | 26.18 | 6 |
| ATOM | 3047 | CA | PRO | A | 671 | 99.182 | 73.472 | 57.378 | 1.00 | 23.70 | 6 |
| ATOM | 3048 | CB | PRO | A | 671 | 99.750 | 73.615 | 55.991 | 1.00 | 25.04 | 6 |
| ATOM | 3049 | CG | PRO | A | 671 | 100.113 | 72.200 | 55.697 | 1.00 | 27.66 | 6 |
| ATOM | 3050 | C | PRO | A | 671 | 98.304 | 74.597 | 57.761 | 1.00 | 23.60 | 6 |
| ATOM | 3051 | O | PRO | A | 671 | 98.259 | 75.162 | 58.853 | 1.00 | 21.85 | 8 |
| ATOM | 3052 | N | TYR | A | 672 | 97.627 | 74.819 | 56.684 | 1.00 | 24.29 | 7 |
| ATOM | 3053 | CA | TYR | A | 672 | 96.646 | 75.770 | 56.457 | 1.00 | 24.50 | 6 |
| ATOM | 3054 | CB | TYR | A | 672 | 96.343 | 75.766 | 54.963 | 1.00 | 22.54 | 6 |
| ATOM | 3055 | CG | TYR | A | 672 | 97.599 | 76.135 | 54.159 | 1.00 | 23.87 | 6 |
| ATOM | 3056 | CD1 | TYR | A | 672 | 98.350 | 75.195 | 53.443 | 1.00 | 20.60 | 6 |
| ATOM | 3057 | CE1 | TYR | A | 672 | 99.517 | 75.581 | 52.748 | 1.00 | 22.81 | 6 |
| ATOM | 3058 | CD2 | TYR | A | 672 | 98.030 | 77.461 | 54.160 | 1.00 | 19.62 | 6 |
| ATOM | 3059 | CE2 | TYR | A | 672 | 99.184 | 77.850 | 53.476 | 1.00 | 23.42 | 6 |
| ATOM | 3060 | CZ | TYR | A | 672 | 99.924 | 76.907 | 52.778 | 1.00 | 21.55 | 6 |
| ATOM | 3061 | OH | TYR | A | 672 | 101.056 | 77.292 | 52.116 | 1.00 | 24.17 | 8 |
| ATOM | 3062 | C | TYR | A | 672 | 95.486 | 75.470 | 57.322 | 1.00 | 24.40 | 6 |
| ATOM | 3063 | O | TYR | A | 672 | 95.115 | 74.324 | 57.590 | 1.00 | 24.35 | 8 |
| ATOM | 3064 | N | HIS | A | 673 | 94.920 | 76.579 | 57.728 | 1.00 | 24.90 | 7 |
| ATOM | 3065 | CA | HIS | A | 673 | 93.787 | 76.632 | 58.582 | 1.00 | 24.47 | 6 |
| ATOM | 3066 | CB | HIS | A | 673 | 93.562 | 78.067 | 59.039 | 1.00 | 24.94 | 6 |
| ATOM | 3067 | CG | HIS | A | 673 | 94.734 | 78.625 | 59.859 | 1.00 | 22.77 | 6 |
| ATOM | 3068 | CD2 | HIS | A | 673 | 95.416 | 79.787 | 59.741 | 1.00 | 25.61 | 6 |
| ATOM | 3069 | ND1 | HIS | A | 673 | 95.296 | 77.961 | 60.929 | 1.00 | 27.26 | 7 |
| ATOM | 3070 | CE1 | HIS | A | 673 | 96.275 | 78.689 | 61.435 | 1.00 | 24.31 | 6 |
| ATOM | 3071 | NE2 | HIS | A | 673 | 96.369 | 79.803 | 60.733 | 1.00 | 25.27 | 7 |
| ATOM | 3072 | C | HIS | A | 673 | 92.569 | 76.157 | 57.858 | 1.00 | 26.37 | 6 |
| ATOM | 3073 | O | HIS | A | 673 | 91.763 | 75.412 | 58.423 | 1.00 | 26.41 | 8 |
| ATOM | 3074 | N | ASP | A | 674 | 92.429 | 76.574 | 56.609 | 1.00 | 27.20 | 7 |
| ATOM | 3075 | CA | ASP | A | 674 | 91.287 | 76.162 | 55.825 | 1.00 | 28.01 | 6 |
| ATOM | 3076 | CB | ASP | A | 674 | 90.886 | 77.275 | 54.856 | 1.00 | 28.51 | 6 |
| ATOM | 3077 | CG | ASP | A | 674 | 89.651 | 76.930 | 54.063 | 1.00 | 29.86 | 6 |
| ATOM | 3078 | OD1 | ASP | A | 674 | 89.246 | 75.750 | 54.065 | 1.00 | 31.34 | 8 |
| ATOM | 3079 | OD2 | ASP | A | 674 | 89.084 | 77.839 | 53.430 | 1.00 | 34.32 | 8 |
| ATOM | 3080 | C | ASP | A | 674 | 91.717 | 74.911 | 55.062 | 1.00 | 28.62 | 6 |
| ATOM | 3081 | O | ASP | A | 674 | 92.607 | 74.964 | 54.212 | 1.00 | 31.42 | 8 |
| ATOM | 3082 | N | SER | A | 675 | 91.100 | 73.783 | 55.380 | 1.00 | 27.13 | 7 |
| ATOM | 3083 | CA | SER | A | 675 | 91.444 | 72.534 | 54.718 | 1.00 | 25.20 | 6 |
| ATOM | 3084 | CB | SER | A | 675 | 92.378 | 71.703 | 55.603 | 1.00 | 23.67 | 6 |
| ATOM | 3085 | OG | SER | A | 675 | 92.408 | 72.215 | 56.927 | 1.00 | 21.86 | 8 |
| ATOM | 3086 | C | SER | A | 675 | 90.195 | 71.734 | 54.441 | 1.00 | 25.11 | 6 |
| ATOM | 3087 | O | SER | A | 675 | 89.117 | 72.040 | 54.958 | 1.00 | 25.84 | 8 |
| ATOM | 3088 | N | ALA | A | 676 | 90.347 | 70.706 | 53.617 | 1.00 | 24.48 | 7 |
| ATOM | 3089 | CA | ALA | A | 676 | 89.236 | 69.840 | 53.270 | 1.00 | 21.55 | 6 |
| ATOM | 3090 | CB | ALA | A | 676 | 89.689 | 68.796 | 52.236 | 1.00 | 21.95 | 6 |
| ATOM | 3091 | C | ALA | A | 676 | 88.748 | 69.149 | 54.548 | 1.00 | 20.66 | 6 |
| ATOM | 3092 | O | ALA | A | 676 | 87.551 | 68.962 | 54.738 | 1.00 | 21.51 | 8 |
| ATOM | 3093 | N | LEU | A | 677 | 89.671 | 68.759 | 55.423 | 1.00 | 21.16 | 7 |
| ATOM | 3094 | CA | LEU | A | 677 | 89.272 | 68.094 | 56.671 | 1.00 | 19.62 | 6 |
| ATOM | 3095 | CB | LEU | A | 677 | 90.502 | 67.635 | 57.474 | 1.00 | 20.42 | 6 |
| ATOM | 3096 | CG | LEU | A | 677 | 90.209 | 67.044 | 58.872 | 1.00 | 20.11 | 6 |
| ATOM | 3097 | CD1 | LEU | A | 677 | 89.240 | 65.856 | 58.760 | 1.00 | 18.78 | 6 |
| ATOM | 3098 | CD2 | LEU | A | 677 | 91.522 | 66.612 | 59.538 | 1.00 | 20.29 | 6 |
| ATOM | 3099 | C | LEU | A | 677 | 88.429 | 69.048 | 57.525 | 1.00 | 17.57 | 6 |
| ATOM | 3100 | O | LEU | A | 677 | 87.387 | 68.665 | 58.054 | 1.00 | 18.12 | 8 |
| ATOM | 3101 | N | ALA | A | 678 | 88.875 | 70.293 | 57.645 | 1.00 | 18.34 | 7 |
| ATOM | 3102 | CA | ALA | A | 678 | 88.142 | 71.272 | 58.448 | 1.00 | 17.93 | 6 |
| ATOM | 3103 | CB | ALA | A | 678 | 88.919 | 72.597 | 58.492 | 1.00 | 17.83 | 6 |
| ATOM | 3104 | C | ALA | A | 678 | 86.757 | 71.484 | 57.864 | 1.00 | 20.58 | 6 |
| ATOM | 3105 | O | ALA | A | 678 | 85.752 | 71.595 | 58.595 | 1.00 | 17.39 | 8 |
| ATOM | 3106 | N | ARG | A | 679 | 86.691 | 71.522 | 56.534 | 1.00 | 21.36 | 7 |
| ATOM | 3107 | CA | ARG | A | 679 | 85.409 | 71.731 | 55.870 | 1.00 | 23.88 | 6 |
| ATOM | 3108 | CB | ARG | A | 679 | 85.614 | 71.984 | 54.377 | 1.00 | 23.79 | 6 |
| ATOM | 3109 | CG | ARG | A | 679 | 86.250 | 73.337 | 54.120 | 1.00 | 27.65 | 6 |
| ATOM | 3110 | CD | ARG | A | 679 | 86.092 | 73.784 | 52.669 | 1.00 | 26.88 | 6 |
| ATOM | 3111 | NE | ARG | A | 679 | 86.528 | 75.169 | 52.523 | 1.00 | 29.31 | 7 |
| ATOM | 3112 | CZ | ARG | A | 679 | 86.198 | 75.955 | 51.503 | 1.00 | 30.12 | 6 |
| ATOM | 3113 | NH1 | ARG | A | 679 | 85.424 | 75.491 | 50.528 | 1.00 | 33.23 | 7 |
| ATOM | 3114 | NH2 | ARG | A | 679 | 86.624 | 77.211 | 51.471 | 1.00 | 28.97 | 7 |
| ATOM | 3115 | C | ARG | A | 679 | 84.529 | 70.530 | 56.084 | 1.00 | 23.78 | 6 |
| ATOM | 3116 | O | ARG | A | 679 | 83.327 | 70.659 | 56.294 | 1.00 | 22.63 | 8 |
| ATOM | 3117 | N | PHE | A | 680 | 85.135 | 69.354 | 56.050 | 1.00 | 22.59 | 7 |
| ATOM | 3118 | CA | PHE | A | 680 | 84.388 | 68.125 | 56.280 | 1.00 | 23.68 | 6 |
| ATOM | 3119 | CB | PHE | A | 680 | 85.306 | 66.916 | 56.136 | 1.00 | 20.56 | 6 |
| ATOM | 3120 | CG | PHE | A | 680 | 84.785 | 65.686 | 56.809 | 1.00 | 22.84 | 6 |
| ATOM | 3121 | CD1 | PHE | A | 680 | 83.718 | 64.972 | 56.260 | 1.00 | 22.03 | 6 |
| ATOM | 3122 | CD2 | PHE | A | 680 | 85.359 | 65.237 | 57.997 | 1.00 | 22.79 | 6 |
| ATOM | 3123 | CE1 | PHE | A | 680 | 83.232 | 63.820 | 56.886 | 1.00 | 22.38 | 6 |
| ATOM | 3124 | CE2 | PHE | A | 680 | 84.881 | 64.089 | 58.631 | 1.00 | 21.92 | 6 |
| ATOM | 3125 | CZ | PHE | A | 680 | 83.812 | 63.377 | 58.070 | 1.00 | 23.29 | 6 |
| ATOM | 3126 | C | PHE | A | 680 | 83.775 | 68.118 | 57.692 | 1.00 | 23.45 | 6 |
| ATOM | 3127 | O | PHE | A | 680 | 82.633 | 67.694 | 57.879 | 1.00 | 24.83 | 8 |
| ATOM | 3128 | N | LEU | A | 681 | 84.540 | 68.555 | 58.686 | 1.00 | 23.38 | 7 |
| ATOM | 3129 | CA | LEU | A | 681 | 84.032 | 68.593 | 60.064 | 1.00 | 24.14 | 6 |
| ATOM | 3130 | CB | LEU | A | 681 | 85.166 | 68.936 | 61.042 | 1.00 | 22.85 | 6 |
| ATOM | 3131 | CG | LEU | A | 681 | 86.239 | 67.841 | 61.125 | 1.00 | 25.25 | 6 |
| ATOM | 3132 | CD1 | LEU | A | 681 | 87.431 | 68.307 | 61.944 | 1.00 | 25.16 | 6 |
| ATOM | 3133 | CD2 | LEU | A | 681 | 85.627 | 66.601 | 61.721 | 1.00 | 23.88 | 6 |
| ATOM | 3134 | C | LEU | A | 681 | 82.916 | 69.631 | 60.169 | 1.00 | 22.54 | 6 |
| ATOM | 3135 | O | LEU | A | 681 | 81.842 | 69.362 | 60.706 | 1.00 | 22.59 | 8 |
| ATOM | 3136 | N | LEU | A | 682 | 83.185 | 70.816 | 59.640 | 1.00 | 24.92 | 7 |
| ATOM | 3137 | CA | LEU | A | 682 | 82.219 | 71.907 | 59.638 | 1.00 | 26.58 | 6 |
| ATOM | 3138 | CB | LEU | A | 682 | 82.733 | 73.035 | 58.746 | 1.00 | 29.58 | 6 |
| ATOM | 3139 | CG | LEU | A | 682 | 82.258 | 74.468 | 58.971 | 1.00 | 33.24 | 6 |
| ATOM | 3140 | CD1 | LEU | A | 682 | 82.784 | 75.351 | 57.857 | 1.00 | 31.20 | 6 |
| ATOM | 3141 | CD2 | LEU | A | 682 | 80.777 | 74.520 | 59.015 | 1.00 | 33.22 | 6 |
| ATOM | 3142 | C | LEU | A | 682 | 80.893 | 71.381 | 59.090 | 1.00 | 28.13 | 6 |
| ATOM | 3143 | O | LEU | A | 682 | 79.844 | 71.522 | 59.725 | 1.00 | 27.44 | 8 |
| ATOM | 3144 | N | LYS | A | 683 | 80.950 | 70.760 | 57.914 | 1.00 | 27.87 | 7 |
| ATOM | 3145 | CA | LYS | A | 683 | 79.762 | 70.211 | 57.260 | 1.00 | 28.96 | 6 |
| ATOM | 3146 | CB | LYS | A | 683 | 80.128 | 69.655 | 55.879 | 1.00 | 30.63 | 6 |
| ATOM | 3147 | CG | LYS | A | 683 | 80.418 | 70.697 | 54.807 | 1.00 | 33.89 | 6 |
| ATOM | 3148 | CD | LYS | A | 683 | 80.898 | 70.009 | 53.524 | 1.00 | 39.63 | 6 |
| ATOM | 3149 | CE | LYS | A | 683 | 82.189 | 69.216 | 53.775 | 1.00 | 41.44 | 6 |
| ATOM | 3150 | NZ | LYS | A | 683 | 82.633 | 68.343 | 52.633 | 1.00 | 43.89 | 7 |
| ATOM | 3151 | C | LYS | A | 683 | 78.998 | 69.128 | 58.027 | 1.00 | 27.32 | 6 |
| ATOM | 3152 | O | LYS | A | 683 | 77.775 | 69.200 | 58.155 | 1.00 | 28.85 | 8 |
| ATOM | 3153 | N | ARG | A | 684 | 79.693 | 68.110 | 58.519 | 1.00 | 25.99 | 7 |
| ATOM | 3154 | CA | ARG | A | 684 | 78.993 | 67.046 | 59.235 | 1.00 | 24.50 | 6 |
| ATOM | 3155 | CB | ARG | A | 684 | 79.923 | 65.845 | 59.465 | 1.00 | 24.46 | 6 |
| ATOM | 3156 | CG | ARG | A | 684 | 80.471 | 65.214 | 58.166 | 1.00 | 24.64 | 6 |
| ATOM | 3157 | CD | ARG | A | 684 | 79.350 | 64.802 | 57.194 | 1.00 | 25.04 | 6 |
| ATOM | 3158 | NE | ARG | A | 684 | 78.507 | 63.740 | 57.727 | 1.00 | 22.67 | 7 |
| ATOM | 3159 | CZ | ARG | A | 684 | 78.685 | 62.444 | 57.487 | 1.00 | 25.99 | 6 |
| ATOM | 3160 | NH1 | ARG | A | 684 | 79.679 | 62.038 | 56.705 | 1.00 | 27.19 | 7 |
| ATOM | 3161 | NH2 | ARG | A | 684 | 77.884 | 61.547 | 58.050 | 1.00 | 23.41 | 7 |
| ATOM | 3162 | C | ARG | A | 684 | 78.409 | 67.550 | 60.559 | 1.00 | 24.11 | 6 |
| ATOM | 3163 | O | ARG | A | 684 | 77.387 | 67.041 | 61.030 | 1.00 | 24.09 | 8 |
| ATOM | 3164 | N | GLY | A | 685 | 79.052 | 68.557 | 61.141 | 1.00 | 24.81 | 7 |
| ATOM | 3165 | CA | GLY | A | 685 | 78.567 | 69.128 | 62.386 | 1.00 | 25.62 | 6 |
| ATOM | 3166 | C | GLY | A | 685 | 77.308 | 69.937 | 62.131 | 1.00 | 26.02 | 6 |
| ATOM | 3167 | O | GLY | A | 685 | 76.388 | 69.942 | 62.950 | 1.00 | 26.21 | 8 |
| ATOM | 3168 | N | LEU | A | 686 | 77.264 | 70.611 | 60.983 | 1.00 | 25.15 | 7 |
| ATOM | 3169 | CA | LEU | A | 686 | 76.101 | 71.416 | 60.611 | 1.00 | 24.42 | 6 |
| ATOM | 3170 | CB | LEU | A | 686 | 76.472 | 72.422 | 59.522 | 1.00 | 21.60 | 6 |
| ATOM | 3171 | CG | LEU | A | 686 | 77.310 | 73.618 | 59.983 | 1.00 | 21.47 | 6 |
| ATOM | 3172 | CD1 | LEU | A | 686 | 77.751 | 74.415 | 58.772 | 1.00 | 21.47 | 6 |
| ATOM | 3173 | CD2 | LEU | A | 686 | 76.500 | 74.496 | 60.936 | 1.00 | 21.23 | 6 |
| ATOM | 3174 | C | LEU | A | 686 | 74.970 | 70.533 | 60.126 | 1.00 | 24.96 | 6 |
| ATOM | 3175 | O | LEU | A | 686 | 73.794 | 70.921 | 60.152 | 1.00 | 23.52 | 8 |
| ATOM | 3176 | N | ARG | A | 687 | 75.326 | 69.331 | 59.703 | 1.00 | 26.61 | 7 |
| ATOM | 3177 | CA | ARG | A | 687 | 74.344 | 68.385 | 59.197 | 1.00 | 28.53 | 6 |
| ATOM | 3178 | CB | ARG | A | 687 | 75.032 | 67.397 | 58.244 | 1.00 | 29.39 | 6 |
| ATOM | 3179 | CG | ARG | A | 687 | 74.174 | 66.190 | 57.858 | 1.00 | 35.52 | 6 |
| ATOM | 3180 | CD | ARG | A | 687 | 74.407 | 65.778 | 56.405 | 1.00 | 38.10 | 6 |
| ATOM | 3181 | NE | ARG | A | 687 | 75.806 | 65.915 | 56.009 | 1.00 | 42.37 | 7 |
| ATOM | 3182 | CZ | ARG | A | 687 | 76.264 | 66.843 | 55.173 | 1.00 | 43.67 | 6 |
| ATOM | 3183 | NH1 | ARG | A | 687 | 75.431 | 67.724 | 54.638 | 1.00 | 48.16 | 7 |
| ATOM | 3184 | NH2 | ARG | A | 687 | 77.554 | 66.895 | 54.875 | 1.00 | 45.13 | 7 |
| ATOM | 3185 | C | ARG | A | 687 | 73.604 | 67.615 | 60.287 | 1.00 | 28.89 | 6 |
| ATOM | 3186 | O | ARG | A | 687 | 72.457 | 67.208 | 60.102 | 1.00 | 29.17 | 8 |
| ATOM | 3187 | N | ASN | A | 688 | 74.247 | 67.425 | 61.429 | 1.00 | 28.40 | 7 |
| ATOM | 3188 | CA | ASN | A | 688 | 73.630 | 66.658 | 62.497 | 1.00 | 26.84 | 6 |
| ATOM | 3189 | CB | ASN | A | 688 | 74.149 | 65.214 | 62.418 | 1.00 | 28.88 | 6 |
| ATOM | 3190 | CG | ASN | A | 688 | 73.729 | 64.371 | 63.596 | 1.00 | 31.06 | 6 |
| ATOM | 3191 | OD1 | ASN | A | 688 | 74.287 | 64.491 | 64.681 | 1.00 | 36.66 | 8 |
| ATOM | 3192 | ND2 | ASN | A | 688 | 72.737 | 63.511 | 63.391 | 1.00 | 33.45 | 7 |
| ATOM | 3193 | C | ASN | A | 688 | 73.887 | 67.267 | 63.872 | 1.00 | 26.97 | 6 |
| ATOM | 3194 | O | ASN | A | 688 | 75.026 | 67.489 | 64.257 | 1.00 | 25.22 | 8 |
| ATOM | 3195 | N | LYS | A | 689 | 72.812 | 67.512 | 64.614 | 1.00 | 27.02 | 7 |
| ATOM | 3196 | CA | LYS | A | 689 | 72.912 | 68.129 | 65.935 | 1.00 | 26.47 | 6 |
| ATOM | 3197 | CB | LYS | A | 689 | 71.512 | 68.346 | 66.535 | 1.00 | 26.83 | 6 |
| ATOM | 3198 | CG | LYS | A | 689 | 70.782 | 69.573 | 65.975 | 1.00 | 30.57 | 6 |
| ATOM | 3199 | CD | LYS | A | 689 | 69.516 | 69.901 | 66.772 | 1.00 | 28.22 | 6 |
| ATOM | 3200 | CE | LYS | A | 689 | 68.833 | 71.170 | 66.239 | 1.00 | 33.75 | 6 |
| ATOM | 3201 | NZ | LYS | A | 689 | 67.757 | 71.682 | 67.149 | 1.00 | 27.87 | 7 |
| ATOM | 3202 | C | LYS | A | 689 | 73.793 | 67.416 | 66.955 | 1.00 | 23.72 | 6 |
| ATOM | 3203 | O | LYS | A | 689 | 74.439 | 68.079 | 67.759 | 1.00 | 23.16 | 8 |
| ATOM | 3204 | N | ARG | A | 690 | 73.821 | 66.086 | 66.938 | 1.00 | 23.70 | 7 |
| ATOM | 3205 | CA | ARG | A | 690 | 74.646 | 65.363 | 67.905 | 1.00 | 23.00 | 6 |
| ATOM | 3206 | CB | ARG | A | 690 | 74.274 | 63.876 | 67.963 | 1.00 | 23.52 | 6 |
| ATOM | 3207 | CG | ARG | A | 690 | 74.962 | 63.174 | 69.153 | 1.00 | 28.33 | 6 |
| ATOM | 3208 | CD | ARG | A | 690 | 74.641 | 61.689 | 69.274 | 1.00 | 28.61 | 6 |
| ATOM | 3209 | NE | ARG | A | 690 | 75.443 | 61.050 | 70.322 | 1.00 | 28.35 | 7 |
| ATOM | 3210 | CZ | ARG | A | 690 | 75.213 | 61.148 | 71.634 | 1.00 | 31.84 | 6 |
| ATOM | 3211 | NH1 | ARG | A | 690 | 74.189 | 61.861 | 72.099 | 1.00 | 30.60 | 7 |
| ATOM | 3212 | NH2 | ARG | A | 690 | 76.015 | 60.528 | 72.492 | 1.00 | 29.15 | 7 |
| ATOM | 3213 | C | ARG | A | 690 | 76.126 | 65.514 | 67.547 | 1.00 | 25.33 | 6 |
| ATOM | 3214 | O | ARG | A | 690 | 76.988 | 65.632 | 68.432 | 1.00 | 23.93 | 8 |
| ATOM | 3215 | N | ILE | A | 691 | 76.431 | 65.503 | 66.250 | 1.00 | 20.50 | 7 |
| ATOM | 3216 | CA | ILE | A | 691 | 77.810 | 65.688 | 65.833 | 1.00 | 19.45 | 6 |
| ATOM | 3217 | CB | ILE | A | 691 | 78.009 | 65.356 | 64.336 | 1.00 | 19.37 | 6 |
| ATOM | 3218 | CG2 | ILE | A | 691 | 79.376 | 65.857 | 63.870 | 1.00 | 18.47 | 6 |
| ATOM | 3219 | CG1 | ILE | A | 691 | 77.881 | 63.838 | 64.133 | 1.00 | 19.36 | 6 |
| ATOM | 3220 | CD1 | ILE | A | 691 | 77.773 | 63.399 | 62.665 | 1.00 | 21.08 | 6 |
| ATOM | 3221 | C | ILE | A | 691 | 78.210 | 67.134 | 66.091 | 1.00 | 20.50 | 6 |
| ATOM | 3222 | O | ILE | A | 691 | 79.326 | 67.405 | 66.535 | 1.00 | 18.96 | 8 |
| ATOM | 3223 | N | GLY | A | 692 | 77.300 | 68.067 | 65.804 | 1.00 | 20.94 | 7 |
| ATOM | 3224 | CA | GLY | A | 692 | 77.595 | 69.474 | 66.022 | 1.00 | 21.64 | 6 |
| ATOM | 3225 | C | GLY | A | 692 | 77.870 | 69.771 | 67.492 | 1.00 | 21.61 | 6 |
| ATOM | 3226 | O | GLY | A | 692 | 78.757 | 70.553 | 67.836 | 1.00 | 21.78 | 8 |
| ATOM | 3227 | N | HIS | A | 693 | 77.097 | 69.133 | 68.358 | 1.00 | 24.01 | 7 |
| ATOM | 3228 | CA | HIS | A | 693 | 77.233 | 69.312 | 69.796 | 1.00 | 23.94 | 6 |
| ATOM | 3229 | CB | HIS | A | 693 | 76.186 | 68.479 | 70.536 | 1.00 | 23.26 | 6 |
| ATOM | 3230 | CG | HIS | A | 693 | 76.541 | 68.215 | 71.966 | 1.00 | 26.61 | 6 |
| ATOM | 3231 | CD2 | HIS | A | 693 | 77.138 | 67.150 | 72.557 | 1.00 | 24.10 | 6 |
| ATOM | 3232 | ND1 | HIS | A | 693 | 76.335 | 69.142 | 72.966 | 1.00 | 23.94 | 7 |
| ATOM | 3233 | CE1 | HIS | A | 693 | 76.789 | 68.661 | 74.111 | 1.00 | 23.91 | 6 |
| ATOM | 3234 | NE2 | HIS | A | 693 | 77.280 | 67.455 | 73.889 | 1.00 | 24.99 | 7 |
| ATOM | 3235 | C | HIS | A | 693 | 78.621 | 68.918 | 70.285 | 1.00 | 23.88 | 6 |
| ATOM | 3236 | O | HIS | A | 693 | 79.284 | 69.689 | 70.986 | 1.00 | 21.95 | 8 |
| ATOM | 3237 | N | PHE | A | 694 | 79.056 | 67.711 | 69.940 | 1.00 | 22.55 | 7 |
| ATOM | 3238 | CA | PHE | A | 694 | 80.369 | 67.260 | 70.372 | 1.00 | 21.87 | 6 |
| ATOM | 3239 | CB | PHE | A | 694 | 80.506 | 65.749 | 70.177 | 1.00 | 21.31 | 6 |
| ATOM | 3240 | CG | PHE | A | 694 | 79.825 | 64.945 | 71.247 | 1.00 | 21.68 | 6 |
| ATOM | 3241 | CD1 | PHE | A | 694 | 78.583 | 64.357 | 71.022 | 1.00 | 21.60 | 6 |
| ATOM | 3242 | CD2 | PHE | A | 694 | 80.427 | 64.785 | 72.493 | 1.00 | 23.10 | 6 |
| ATOM | 3243 | CE1 | PHE | A | 694 | 77.950 | 63.619 | 72.019 | 1.00 | 20.83 | 6 |
| ATOM | 3244 | CE2 | PHE | A | 694 | 79.800 | 64.043 | 73.506 | 1.00 | 19.98 | 6 |
| ATOM | 3245 | CZ | PHE | A | 694 | 78.559 | 63.460 | 73.266 | 1.00 | 21.79 | 6 |
| ATOM | 3246 | C | PHE | A | 694 | 81.491 | 68.020 | 69.660 | 1.00 | 23.01 | 6 |
| ATOM | 3247 | O | PHE | A | 694 | 82.529 | 68.302 | 70.257 | 1.00 | 22.44 | 8 |
| ATOM | 3248 | N | LEU | A | 695 | 81.285 | 68.363 | 68.391 | 1.00 | 20.00 | 7 |
| ATOM | 3249 | CA | LEU | A | 695 | 82.290 | 69.128 | 67.664 | 1.00 | 18.25 | 6 |
| ATOM | 3250 | CB | LEU | A | 695 | 81.786 | 69.470 | 66.261 | 1.00 | 19.55 | 6 |
| ATOM | 3251 | CG | LEU | A | 695 | 82.786 | 70.204 | 65.356 | 1.00 | 21.85 | 6 |
| ATOM | 3252 | CD1 | LEU | A | 695 | 83.968 | 69.303 | 65.065 | 1.00 | 20.89 | 6 |
| ATOM | 3253 | CD2 | LEU | A | 695 | 82.108 | 70.626 | 64.050 | 1.00 | 21.95 | 6 |
| ATOM | 3254 | C | LEU | A | 695 | 82.552 | 70.427 | 68.447 | 1.00 | 19.08 | 6 |
| ATOM | 3255 | O | LEU | A | 695 | 83.694 | 70.878 | 68.568 | 1.00 | 16.96 | 8 |
| ATOM | 3256 | N | PHE | A | 696 | 81.479 | 71.019 | 68.972 | 1.00 | 18.16 | 7 |
| ATOM | 3257 | CA | PHE | A | 696 | 81.567 | 72.258 | 69.761 | 1.00 | 19.05 | 6 |
| ATOM | 3258 | CB | PHE | A | 696 | 80.172 | 72.649 | 70.287 | 1.00 | 17.13 | 6 |
| ATOM | 3259 | CG | PHE | A | 696 | 80.176 | 73.824 | 71.224 | 1.00 | 21.10 | 6 |
| ATOM | 3260 | CD1 | PHE | A | 696 | 80.023 | 75.124 | 70.742 | 1.00 | 20.90 | 6 |
| ATOM | 3261 | CD2 | PHE | A | 696 | 80.320 | 73.631 | 72.604 | 1.00 | 18.88 | 6 |
| ATOM | 3262 | CE1 | PHE | A | 696 | 80.011 | 76.206 | 71.606 | 1.00 | 23.80 | 6 |
| ATOM | 3263 | CE2 | PHE | A | 696 | 80.310 | 74.708 | 73.475 | 1.00 | 22.82 | 6 |
| ATOM | 3264 | CZ | PHE | A | 696 | 80.155 | 76.000 | 72.984 | 1.00 | 22.92 | 6 |
| ATOM | 3265 | C | PHE | A | 696 | 82.511 | 72.067 | 70.947 | 1.00 | 18.13 | 6 |
| ATOM | 3266 | O | PHE | A | 696 | 83.480 | 72.801 | 71.109 | 1.00 | 19.82 | 8 |
| ATOM | 3267 | N | TRP | A | 697 | 82.218 | 71.066 | 71.770 | 1.00 | 17.27 | 7 |
| ATOM | 3268 | CA | TRP | A | 697 | 83.026 | 70.810 | 72.960 | 1.00 | 21.12 | 6 |
| ATOM | 3269 | CB | TRP | A | 697 | 82.293 | 69.825 | 73.876 | 1.00 | 19.20 | 6 |
| ATOM | 3270 | CG | TRP | A | 697 | 81.111 | 70.497 | 74.525 | 1.00 | 24.42 | 6 |
| ATOM | 3271 | CD2 | TRP | A | 697 | 81.165 | 71.541 | 75.502 | 1.00 | 23.09 | 6 |
| ATOM | 3272 | CE2 | TRP | A | 697 | 79.837 | 71.947 | 75.759 | 1.00 | 24.00 | 6 |
| ATOM | 3273 | CE3 | TRP | A | 697 | 82.212 | 72.175 | 76.186 | 1.00 | 24.06 | 6 |
| ATOM | 3274 | CD1 | TRP | A | 697 | 79.781 | 70.309 | 74.241 | 1.00 | 24.37 | 6 |
| ATOM | 3275 | NE1 | TRP | A | 697 | 79.010 | 71.181 | 74.978 | 1.00 | 22.76 | 7 |
| ATOM | 3276 | CZ2 | TRP | A | 697 | 79.529 | 72.956 | 76.671 | 1.00 | 25.31 | 6 |
| ATOM | 3277 | CZ3 | TRP | A | 697 | 81.906 | 73.179 | 77.092 | 1.00 | 25.77 | 6 |
| ATOM | 3278 | CH2 | TRP | A | 697 | 80.575 | 73.560 | 77.328 | 1.00 | 24.27 | 6 |
| ATOM | 3279 | C | TRP | A | 697 | 84.459 | 70.364 | 72.698 | 1.00 | 20.13 | 6 |
| ATOM | 3280 | O | TRP | A | 697 | 85.359 | 70.718 | 73.468 | 1.00 | 18.09 | 8 |
| ATOM | 3281 | N | PHE | A | 698 | 84.676 | 69.620 | 71.610 | 1.00 | 18.95 | 7 |
| ATOM | 3282 | CA | PHE | A | 698 | 86.019 | 69.155 | 71.254 | 1.00 | 19.41 | 6 |
| ATOM | 3283 | CB | PHE | A | 698 | 85.965 | 68.062 | 70.170 | 1.00 | 17.87 | 6 |
| ATOM | 3284 | CG | PHE | A | 698 | 85.440 | 66.721 | 70.654 | 1.00 | 20.65 | 6 |
| ATOM | 3285 | CD1 | PHE | A | 698 | 86.268 | 65.600 | 70.660 | 1.00 | 21.11 | 6 |
| ATOM | 3286 | CD2 | PHE | A | 698 | 84.131 | 66.579 | 71.125 | 1.00 | 22.12 | 6 |
| ATOM | 3287 | CE1 | PHE | A | 698 | 85.815 | 64.360 | 71.125 | 1.00 | 20.90 | 6 |
| ATOM | 3288 | CE2 | PHE | A | 698 | 83.659 | 65.343 | 71.595 | 1.00 | 22.84 | 6 |
| ATOM | 3289 | CZ | PHE | A | 698 | 84.505 | 64.231 | 71.593 | 1.00 | 22.20 | 6 |
| ATOM | 3290 | C | PHE | A | 698 | 86.853 | 70.328 | 70.746 | 1.00 | 18.83 | 6 |
| ATOM | 3291 | O | PHE | A | 698 | 88.054 | 70.389 | 70.984 | 1.00 | 20.49 | 8 |
| ATOM | 3292 | N | LEU | A | 699 | 86.227 | 71.254 | 70.022 | 1.00 | 19.21 | 7 |
| ATOM | 3293 | CA | LEU | A | 699 | 86.947 | 72.420 | 69.528 | 1.00 | 17.18 | 6 |
| ATOM | 3294 | CB | LEU | A | 699 | 86.145 | 73.136 | 68.420 | 1.00 | 19.36 | 6 |
| ATOM | 3295 | CG | LEU | A | 699 | 86.012 | 72.460 | 67.045 | 1.00 | 18.10 | 6 |
| ATOM | 3296 | CD1 | LEU | A | 699 | 85.039 | 73.244 | 66.146 | 1.00 | 19.34 | 6 |
| ATOM | 3297 | CD2 | LEU | A | 699 | 87.377 | 72.392 | 66.394 | 1.00 | 19.57 | 6 |
| ATOM | 3298 | C | LEU | A | 699 | 87.197 | 73.396 | 70.683 | 1.00 | 19.71 | 6 |
| ATOM | 3299 | O | LEU | A | 699 | 88.300 | 73.924 | 70.833 | 1.00 | 20.65 | 8 |
| ATOM | 3300 | N | ARG | A | 700 | 86.177 | 73.613 | 71.509 | 1.00 | 19.34 | 7 |
| ATOM | 3301 | CA | ARG | A | 700 | 86.301 | 74.547 | 72.629 | 1.00 | 21.27 | 6 |
| ATOM | 3302 | CB | ARG | A | 700 | 84.936 | 74.715 | 73.344 | 1.00 | 18.59 | 6 |
| ATOM | 3303 | CG | ARG | A | 700 | 84.893 | 75.913 | 74.328 | 1.00 | 22.00 | 6 |
| ATOM | 3304 | CD | ARG | A | 700 | 83.470 | 76.244 | 74.827 | 1.00 | 23.77 | 6 |
| ATOM | 3305 | NE | ARG | A | 700 | 83.512 | 77.308 | 75.825 | 1.00 | 27.31 | 7 |
| ATOM | 3306 | CZ | ARG | A | 700 | 83.871 | 77.139 | 77.097 | 1.00 | 30.05 | 6 |
| ATOM | 3307 | NH1 | ARG | A | 700 | 84.207 | 75.932 | 77.543 | 1.00 | 28.62 | 7 |
| ATOM | 3308 | NH2 | ARG | A | 700 | 83.932 | 78.190 | 77.917 | 1.00 | 27.43 | 7 |
| ATOM | 3309 | C | ARG | A | 700 | 87.379 | 74.067 | 73.614 | 1.00 | 20.58 | 6 |
| ATOM | 3310 | O | ARG | A | 700 | 88.092 | 74.870 | 74.220 | 1.00 | 21.40 | 8 |
| ATOM | 3311 | N | SER | A | 701 | 87.510 | 72.750 | 73.737 | 1.00 | 21.94 | 7 |
| ATOM | 3312 | CA | SER | A | 701 | 88.490 | 72.145 | 74.624 | 1.00 | 23.81 | 6 |
| ATOM | 3313 | CB | SER | A | 701 | 88.365 | 70.623 | 74.585 | 1.00 | 24.36 | 6 |
| ATOM | 3314 | OG | SER | A | 701 | 89.447 | 70.040 | 75.277 | 1.00 | 26.31 | 8 |
| ATOM | 3315 | C | SER | A | 701 | 89.910 | 72.540 | 74.258 | 1.00 | 24.59 | 6 |
| ATOM | 3316 | O | SER | A | 701 | 90.751 | 72.791 | 75.135 | 1.00 | 23.14 | 8 |
| ATOM | 3317 | N | GLU | A | 702 | 90.180 | 72.587 | 72.959 | 1.00 | 23.77 | 7 |
| ATOM | 3318 | CA | GLU | A | 702 | 91.500 | 72.969 | 72.483 | 1.00 | 24.54 | 6 |
| ATOM | 3319 | CB | GLU | A | 702 | 91.763 | 72.427 | 71.067 | 1.00 | 22.85 | 6 |
| ATOM | 3320 | CG | GLU | A | 702 | 91.865 | 70.908 | 70.992 | 1.00 | 26.41 | 6 |
| ATOM | 3321 | CD | GLU | A | 702 | 93.026 | 70.334 | 71.785 | 1.00 | 31.08 | 6 |
| ATOM | 3322 | OE1 | GLU | A | 702 | 94.005 | 71.074 | 71.995 | 1.00 | 34.68 | 8 |
| ATOM | 3323 | OE2 | GLU | A | 702 | 92.940 | 69.162 | 72.204 | 1.00 | 28.17 | 8 |
| ATOM | 3324 | C | GLU | A | 702 | 91.673 | 74.492 | 72.482 | 1.00 | 20.46 | 6 |
| ATOM | 3325 | O | GLU | A | 702 | 92.753 | 74.992 | 72.765 | 1.00 | 23.16 | 8 |
| ATOM | 3326 | N | ILE | A | 703 | 90.614 | 75.225 | 72.162 | 1.00 | 19.71 | 7 |
| ATOM | 3327 | CA | ILE | A | 703 | 90.695 | 76.682 | 72.140 | 1.00 | 20.51 | 6 |
| ATOM | 3328 | CB | ILE | A | 703 | 89.375 | 77.300 | 71.588 | 1.00 | 22.47 | 6 |
| ATOM | 3329 | CG2 | ILE | A | 703 | 89.411 | 78.807 | 71.644 | 1.00 | 19.59 | 6 |
| ATOM | 3330 | CG1 | ILE | A | 703 | 89.155 | 76.825 | 70.143 | 1.00 | 18.98 | 6 |
| ATOM | 3331 | CD1 | ILE | A | 703 | 87.720 | 76.945 | 69.662 | 1.00 | 21.54 | 6 |
| ATOM | 3332 | C | ILE | A | 703 | 90.994 | 77.243 | 73.537 | 1.00 | 22.72 | 6 |
| ATOM | 3333 | O | ILE | A | 703 | 91.671 | 78.264 | 73.669 | 1.00 | 24.07 | 8 |
| ATOM | 3334 | N | ALA | A | 704 | 90.478 | 76.580 | 74.572 | 1.00 | 24.28 | 7 |
| ATOM | 3335 | CA | ALA | A | 704 | 90.652 | 77.010 | 75.957 | 1.00 | 26.37 | 6 |
| ATOM | 3336 | CB | ALA | A | 704 | 89.600 | 76.341 | 76.825 | 1.00 | 20.55 | 6 |
| ATOM | 3337 | C | ALA | A | 704 | 92.036 | 76.742 | 76.518 | 1.00 | 28.55 | 6 |
| ATOM | 3338 | O | ALA | A | 704 | 92.429 | 77.357 | 77.509 | 1.00 | 30.75 | 8 |
| ATOM | 3339 | N | GLN | A | 705 | 92.806 | 75.836 | 75.906 | 1.00 | 29.09 | 7 |
| ATOM | 3340 | CA | GLN | A | 705 | 94.087 | 75.490 | 76.529 | 1.00 | 29.22 | 6 |
| ATOM | 3341 | CB | GLN | A | 705 | 93.914 | 74.156 | 77.256 | 1.00 | 29.33 | 6 |
| ATOM | 3342 | CG | GLN | A | 705 | 93.412 | 73.064 | 76.336 | 1.00 | 31.10 | 6 |
| ATOM | 3343 | CD | GLN | A | 705 | 92.960 | 71.824 | 77.083 | 1.00 | 33.03 | 6 |
| ATOM | 3344 | OE1 | GLN | A | 705 | 93.757 | 71.180 | 77.763 | 1.00 | 33.61 | 8 |
| ATOM | 3345 | NE2 | GLN | A | 705 | 91.735 | 71.328 | 77.112 | 1.00 | 30.98 | 7 |
| ATOM | 3346 | C | GLN | A | 705 | 95.295 | 75.371 | 75.602 | 1.00 | 29.84 | 6 |
| ATOM | 3347 | O | GLN | A | 705 | 96.438 | 75.243 | 76.070 | 1.00 | 32.83 | 8 |
| ATOM | 3348 | N | SER | A | 706 | 95.078 | 75.411 | 74.311 | 1.00 | 27.20 | 7 |
| ATOM | 3349 | CA | SER | A | 706 | 96.225 | 75.233 | 73.453 | 1.00 | 25.05 | 6 |
| ATOM | 3350 | CB | SER | A | 706 | 95.926 | 74.156 | 72.429 | 1.00 | 25.33 | 6 |
| ATOM | 3351 | OG | SER | A | 706 | 96.921 | 74.124 | 71.417 | 1.00 | 23.33 | 8 |
| ATOM | 3352 | C | SER | A | 706 | 96.637 | 76.521 | 72.790 | 1.00 | 23.58 | 6 |
| ATOM | 3353 | O | SER | A | 706 | 95.958 | 77.021 | 71.898 | 1.00 | 21.82 | 8 |
| ATOM | 3354 | N | ARG | A | 707 | 97.428 | 76.846 | 73.355 | 1.00 | 23.04 | 7 |
| ATOM | 3355 | CA | ARG | A | 707 | 97.968 | 78.121 | 72.930 | 1.00 | 23.28 | 6 |
| ATOM | 3356 | CB | ARG | A | 707 | 99.034 | 78.646 | 73.895 | 1.00 | 25.15 | 6 |
| ATOM | 3357 | CG | ARG | A | 707 | 98.626 | 78.589 | 75.359 | 1.00 | 32.56 | 6 |
| ATOM | 3358 | CD | ARG | A | 707 | 99.572 | 79.413 | 76.209 | 1.00 | 37.92 | 6 |
| ATOM | 3359 | NE | ARG | A | 707 | 99.218 | 80.832 | 76.217 | 1.00 | 42.94 | 7 |
| ATOM | 3360 | CZ | ARG | A | 707 | 98.787 | 81.483 | 77.299 | 1.00 | 46.51 | 6 |
| ATOM | 3361 | NH1 | ARG | A | 707 | 98.655 | 80.834 | 78.452 | 1.00 | 46.28 | 7 |
| ATOM | 3362 | NH2 | ARG | A | 707 | 98.503 | 82.780 | 77.240 | 1.00 | 45.86 | 7 |
| ATOM | 3363 | C | ARG | A | 707 | 98.555 | 77.945 | 71.530 | 1.00 | 23.81 | 6 |
| ATOM | 3364 | O | ARG | A | 707 | 98.509 | 78.857 | 70.700 | 1.00 | 21.00 | 8 |
| ATOM | 3365 | N | HIS | A | 708 | 99.170 | 76.722 | 70.951 | 1.00 | 22.57 | 7 |
| ATOM | 3366 | CA | HIS | A | 708 | 99.610 | 76.301 | 69.612 | 1.00 | 22.49 | 6 |
| ATOM | 3367 | CB | HIS | A | 708 | 100.333 | 74.942 | 69.681 | 1.00 | 24.79 | 6 |
| ATOM | 3368 | CG | HIS | A | 708 | 101.510 | 74.920 | 70.616 | 1.00 | 23.46 | 6 |
| ATOM | 3369 | CD2 | HIS | A | 708 | 102.844 | 74.938 | 70.371 | 1.00 | 25.51 | 6 |
| ATOM | 3370 | ND1 | HIS | A | 708 | 101.375 | 74.888 | 71.989 | 1.00 | 23.49 | 7 |
| ATOM | 3371 | CE1 | HIS | A | 708 | 102.573 | 74.889 | 72.548 | 1.00 | 25.13 | 6 |
| ATOM | 3372 | NE2 | HIS | A | 708 | 103.482 | 74.920 | 71.591 | 1.00 | 27.29 | 7 |
| ATOM | 3373 | C | HIS | A | 708 | 98.505 | 76.227 | 68.541 | 1.00 | 22.67 | 6 |
| ATOM | 3374 | O | HIS | A | 708 | 98.768 | 76.494 | 67.363 | 1.00 | 19.35 | 8 |
| ATOM | 3375 | N | TYR | A | 709 | 97.284 | 75.873 | 68.933 | 1.00 | 19.56 | 7 |
| ATOM | 3376 | CA | TYR | A | 709 | 96.193 | 75.752 | 67.954 | 1.00 | 22.22 | 6 |
| ATOM | 3377 | CB | TYR | A | 709 | 95.896 | 74.266 | 67.691 | 1.00 | 23.12 | 6 |
| ATOM | 3378 | CG | TYR | A | 709 | 97.069 | 73.525 | 67.082 | 1.00 | 22.87 | 6 |
| ATOM | 3379 | CD1 | TYR | A | 709 | 97.938 | 72.769 | 67.874 | 1.00 | 21.66 | 6 |
| ATOM | 3380 | CE1 | TYR | A | 709 | 99.057 | 72.130 | 67.304 | 1.00 | 22.18 | 6 |
| ATOM | 3381 | CD2 | TYR | A | 709 | 97.336 | 73.631 | 65.719 | 1.00 | 22.51 | 6 |
| ATOM | 3382 | CE2 | TYR | A | 709 | 98.439 | 73.013 | 65.144 | 1.00 | 20.91 | 6 |
| ATOM | 3383 | CZ | TYR | A | 709 | 99.295 | 72.265 | 65.940 | 1.00 | 20.05 | 6 |
| ATOM | 3384 | OH | TYR | A | 709 | 100.391 | 71.690 | 65.360 | 1.00 | 22.66 | 8 |
| ATOM | 3385 | C | TYR | A | 709 | 94.866 | 76.460 | 68.251 | 1.00 | 19.40 | 6 |
| ATOM | 3386 | O | TYR | A | 709 | 93.936 | 76.395 | 67.436 | 1.00 | 19.16 | 8 |
| ATOM | 3387 | N | GLN | A | 710 | 94.762 | 77.143 | 69.390 | 1.00 | 19.21 | 7 |
| ATOM | 3388 | CA | GLN | A | 710 | 93.496 | 77.796 | 69.712 | 1.00 | 21.01 | 6 |
| ATOM | 3389 | CB | GLN | A | 710 | 93.578 | 78.545 | 71.051 | 1.00 | 21.79 | 6 |
| ATOM | 3390 | CG | GLN | A | 710 | 94.201 | 79.930 | 70.981 | 1.00 | 22.50 | 6 |
| ATOM | 3391 | CD | GLN | A | 710 | 94.564 | 80.477 | 72.357 | 1.00 | 25.58 | 6 |
| ATOM | 3392 | OE1 | GLN | A | 710 | 95.321 | 81.431 | 72.466 | 1.00 | 28.28 | 8 |
| ATOM | 3393 | NE2 | GLN | A | 710 | 94.023 | 79.867 | 73.407 | 1.00 | 22.40 | 7 |
| ATOM | 3394 | C | GLN | A | 710 | 93.022 | 78.744 | 68.616 | 1.00 | 18.17 | 6 |
| ATOM | 3395 | O | GLN | A | 710 | 91.825 | 78.850 | 68.382 | 1.00 | 21.47 | 8 |
| ATOM | 3396 | N | GLN | A | 711 | 93.947 | 79.407 | 67.925 | 1.00 | 19.34 | 7 |
| ATOM | 3397 | CA | GLN | A | 711 | 93.568 | 80.352 | 66.868 | 1.00 | 20.41 | 6 |
| ATOM | 3398 | CB | GLN | A | 711 | 94.789 | 81.119 | 66.371 | 1.00 | 22.98 | 6 |
| ATOM | 3399 | CG | GLN | A | 711 | 94.965 | 82.491 | 66.990 | 1.00 | 24.72 | 6 |
| ATOM | 3400 | CD | GLN | A | 711 | 96.273 | 83.121 | 66.573 | 1.00 | 26.38 | 6 |
| ATOM | 3401 | OE1 | GLN | A | 711 | 97.350 | 82.650 | 66.956 | 1.00 | 26.35 | 8 |
| ATOM | 3402 | NE2 | GLN | A | 711 | 96.196 | 84.176 | 65.760 | 1.00 | 23.68 | 7 |
| ATOM | 3403 | C | GLN | A | 711 | 92.891 | 79.658 | 65.687 | 1.00 | 21.02 | 6 |
| ATOM | 3404 | O | GLN | A | 711 | 91.822 | 80.089 | 65.212 | 1.00 | 16.34 | 8 |
| ATOM | 3405 | N | ARG | A | 712 | 93.525 | 78.598 | 65.195 | 1.00 | 20.62 | 7 |
| ATOM | 3406 | CA | ARG | A | 712 | 92.949 | 77.830 | 64.095 | 1.00 | 18.44 | 6 |
| ATOM | 3407 | CB | ARG | A | 712 | 93.897 | 76.713 | 63.645 | 1.00 | 20.40 | 6 |
| ATOM | 3408 | CG | ARG | A | 712 | 93.266 | 75.746 | 62.631 | 1.00 | 20.81 | 6 |
| ATOM | 3409 | CD | ARG | A | 712 | 94.254 | 74.662 | 62.182 | 1.00 | 23.42 | 6 |
| ATOM | 3410 | NE | ARG | A | 712 | 95.397 | 75.187 | 61.424 | 1.00 | 20.56 | 7 |
| ATOM | 3411 | CZ | ARG | A | 712 | 96.423 | 74.436 | 61.023 | 1.00 | 24.19 | 6 |
| ATOM | 3412 | NH1 | ARG | A | 712 | 96.448 | 73.136 | 61.314 | 1.00 | 23.05 | 7 |
| ATOM | 3413 | NH2 | ARG | A | 712 | 97.415 | 74.965 | 60.318 | 1.00 | 21.46 | 7 |
| ATOM | 3414 | C | ARG | A | 712 | 91.627 | 77.216 | 64.532 | 1.00 | 19.56 | 6 |
| ATOM | 3415 | O | ARG | A | 712 | 90.624 | 77.337 | 63.834 | 1.00 | 20.88 | 8 |
| ATOM | 3416 | N | PHE | A | 713 | 91.598 | 76.568 | 65.695 | 1.00 | 17.14 | 7 |
| ATOM | 3417 | CA | PHE | A | 713 | 90.355 | 75.955 | 66.138 | 1.00 | 17.40 | 6 |
| ATOM | 3418 | CB | PHE | A | 713 | 90.588 | 75.081 | 67.391 | 1.00 | 23.04 | 6 |
| ATOM | 3419 | CG | PHE | A | 713 | 91.271 | 73.758 | 67.106 | 1.00 | 23.61 | 6 |
| ATOM | 3420 | CD1 | PHE | A | 713 | 92.475 | 73.434 | 67.714 | 1.00 | 29.71 | 6 |
| ATOM | 3421 | CD2 | PHE | A | 713 | 90.698 | 72.834 | 66.238 | 1.00 | 29.09 | 6 |
| ATOM | 3422 | CE1 | PHE | A | 713 | 93.104 | 72.205 | 67.465 | 1.00 | 28.30 | 6 |
| ATOM | 3423 | CE2 | PHE | A | 713 | 91.318 | 71.603 | 65.981 | 1.00 | 28.33 | 6 |
| ATOM | 3424 | CZ | PHE | A | 713 | 92.520 | 71.290 | 66.595 | 1.00 | 26.28 | 6 |
| ATOM | 3425 | C | PHE | A | 713 | 89.236 | 76.979 | 66.416 | 1.00 | 19.45 | 6 |
| ATOM | 3426 | O | PHE | A | 713 | 88.060 | 76.664 | 66.251 | 1.00 | 18.59 | 8 |
| ATOM | 3427 | N | ALA | A | 714 | 89.588 | 78.193 | 66.837 | 1.00 | 17.12 | 7 |
| ATOM | 3428 | CA | ALA | A | 714 | 88.559 | 79.195 | 67.129 | 1.00 | 19.17 | 6 |
| ATOM | 3429 | CB | ALA | A | 714 | 89.184 | 80.426 | 67.826 | 1.00 | 14.73 | 6 |
| ATOM | 3430 | C | ALA | A | 714 | 87.834 | 79.629 | 65.851 | 1.00 | 18.89 | 6 |
| ATOM | 3431 | O | ALA | A | 714 | 86.634 | 79.879 | 65.856 | 1.00 | 19.59 | 8 |
| ATOM | 3432 | N | VAL | A | 715 | 88.575 | 79.700 | 64.758 | 1.00 | 22.18 | 7 |
| ATOM | 3433 | CA | VAL | A | 715 | 88.011 | 80.109 | 63.480 | 1.00 | 23.92 | 6 |
| ATOM | 3434 | CB | VAL | A | 715 | 89.142 | 80.379 | 62.459 | 1.00 | 27.54 | 6 |
| ATOM | 3435 | CG1 | VAL | A | 715 | 88.568 | 80.590 | 61.074 | 1.00 | 28.34 | 6 |
| ATOM | 3436 | CG2 | VAL | A | 715 | 89.919 | 81.623 | 62.881 | 1.00 | 29.51 | 6 |
| ATOM | 3437 | C | VAL | A | 715 | 87.043 | 79.059 | 62.944 | 1.00 | 24.22 | 6 |
| ATOM | 3438 | O | VAL | A | 715 | 86.005 | 79.391 | 62.381 | 1.00 | 26.51 | 8 |
| ATOM | 3439 | N | ILE | A | 716 | 87.377 | 77.787 | 63.115 | 1.00 | 21.55 | 7 |
| ATOM | 3440 | CA | ILE | A | 716 | 86.488 | 76.731 | 62.655 | 1.00 | 20.82 | 6 |
| ATOM | 3441 | CB | ILE | A | 716 | 87.188 | 75.361 | 62.699 | 1.00 | 21.53 | 6 |
| ATOM | 3442 | CG2 | ILE | A | 716 | 86.212 | 74.265 | 62.273 | 1.00 | 20.74 | 6 |
| ATOM | 3443 | CG1 | ILE | A | 716 | 88.431 | 75.394 | 61.800 | 1.00 | 20.45 | 6 |
| ATOM | 3444 | CD1 | ILE | A | 716 | 89.329 | 74.161 | 61.936 | 1.00 | 25.64 | 6 |
| ATOM | 3445 | C | ILE | A | 716 | 85.236 | 76.675 | 63.529 | 1.00 | 21.15 | 6 |
| ATOM | 3446 | O | ILE | A | 716 | 84.141 | 76.423 | 63.038 | 1.00 | 19.24 | 8 |
| ATOM | 3447 | N | LEU | A | 717 | 85.397 | 76.916 | 64.830 | 1.00 | 20.88 | 7 |
| ATOM | 3448 | CA | LEU | A | 717 | 84.254 | 76.877 | 65.736 | 1.00 | 23.03 | 6 |
| ATOM | 3449 | CB | LEU | A | 717 | 84.704 | 77.003 | 67.201 | 1.00 | 22.06 | 6 |
| ATOM | 3450 | CG | LEU | A | 717 | 83.956 | 76.220 | 68.296 | 1.00 | 27.45 | 6 |
| ATOM | 3451 | CD1 | LEU | A | 717 | 84.009 | 77.018 | 69.581 | 1.00 | 24.72 | 6 |
| ATOM | 3452 | CD2 | LEU | A | 717 | 82.511 | 75.941 | 67.926 | 1.00 | 26.80 | 6 |
| ATOM | 3453 | C | LEU | A | 717 | 83.314 | 78.027 | 65.397 | 1.00 | 22.17 | 6 |
| ATOM | 3454 | O | LEU | A | 717 | 82.108 | 77.834 | 65.316 | 1.00 | 21.03 | 8 |
| ATOM | 3455 | N | GLU | A | 718 | 83.861 | 79.230 | 65.216 | 1.00 | 25.23 | 7 |
| ATOM | 3456 | CA | GLU | A | 718 | 83.009 | 80.366 | 64.871 | 1.00 | 24.56 | 6 |
| ATOM | 3457 | CB | GLU | A | 718 | 83.824 | 81.624 | 64.566 | 1.00 | 24.34 | 6 |
| ATOM | 3458 | CG | GLU | A | 718 | 82.917 | 82.743 | 64.019 | 1.00 | 27.28 | 6 |
| ATOM | 3459 | CD | GLU | A | 718 | 83.660 | 83.895 | 63.388 | 1.00 | 30.88 | 6 |
| ATOM | 3460 | OE1 | GLU | A | 718 | 84.622 | 83.646 | 62.630 | 1.00 | 30.95 | 8 |
| ATOM | 3461 | OE2 | GLU | A | 718 | 83.261 | 85.056 | 63.633 | 1.00 | 34.67 | 8 |
| ATOM | 3462 | C | GLU | A | 718 | 82.182 | 80.019 | 63.631 | 1.00 | 24.39 | 6 |
| ATOM | 3463 | O | GLU | A | 718 | 80.984 | 80.284 | 63.581 | 1.00 | 25.59 | 8 |
| ATOM | 3464 | N | ALA | A | 719 | 82.827 | 79.423 | 62.633 | 1.00 | 24.55 | 7 |
| ATOM | 3465 | CA | ALA | A | 719 | 82.132 | 79.047 | 61.404 | 1.00 | 23.14 | 6 |
| ATOM | 3466 | CB | ALA | A | 719 | 83.104 | 78.395 | 60.424 | 1.00 | 19.50 | 6 |
| ATOM | 3467 | C | ALA | A | 719 | 80.951 | 78.119 | 61.682 | 1.00 | 22.38 | 6 |
| ATOM | 3468 | O | ALA | A | 719 | 79.888 | 78.248 | 61.067 | 1.00 | 22.07 | 8 |
| ATOM | 3469 | N | TYR | A | 720 | 81.135 | 77.178 | 62.599 | 1.00 | 23.16 | 7 |
| ATOM | 3470 | CA | TYR | A | 720 | 80.064 | 76.254 | 62.958 | 1.00 | 22.54 | 6 |
| ATOM | 3471 | CB | TYR | A | 720 | 80.610 | 75.110 | 63.827 | 1.00 | 21.12 | 6 |
| ATOM | 3472 | CG | TYR | A | 720 | 79.521 | 74.270 | 64.466 | 1.00 | 20.52 | 6 |
| ATOM | 3473 | CD1 | TYR | A | 720 | 78.816 | 73.314 | 63.731 | 1.00 | 19.63 | 6 |
| ATOM | 3474 | CE1 | TYR | A | 720 | 77.751 | 72.604 | 64.305 | 1.00 | 20.33 | 6 |
| ATOM | 3475 | CD2 | TYR | A | 720 | 79.143 | 74.492 | 65.796 | 1.00 | 19.63 | 6 |
| ATOM | 3476 | CE2 | TYR | A | 720 | 78.095 | 73.800 | 66.374 | 1.00 | 20.31 | 6 |
| ATOM | 3477 | CZ | TYR | A | 720 | 77.398 | 72.858 | 65.631 | 1.00 | 21.58 | 6 |
| ATOM | 3478 | OH | TYR | A | 720 | 76.345 | 72.189 | 66.214 | 1.00 | 23.51 | 8 |
| ATOM | 3479 | C | TYR | A | 720 | 78.957 | 76.989 | 63.732 | 1.00 | 23.72 | 6 |
| ATOM | 3480 | O | TYR | A | 720 | 77.768 | 76.774 | 63.496 | 1.00 | 22.32 | 8 |
| ATOM | 3481 | N | LEU | A | 721 | 79.348 | 77.853 | 64.661 | 1.00 | 24.22 | 7 |
| ATOM | 3482 | CA | LEU | A | 721 | 78.355 | 78.573 | 65.452 | 1.00 | 26.99 | 6 |
| ATOM | 3483 | CB | LEU | A | 721 | 79.039 | 79.410 | 66.537 | 1.00 | 24.63 | 6 |
| ATOM | 3484 | CG | LEU | A | 721 | 79.661 | 78.576 | 67.673 | 1.00 | 25.99 | 6 |
| ATOM | 3485 | CD1 | LEU | A | 721 | 80.298 | 79.490 | 68.709 | 1.00 | 25.48 | 6 |
| ATOM | 3486 | CD2 | LEU | A | 721 | 78.580 | 77.725 | 68.323 | 1.00 | 22.70 | 6 |
| ATOM | 3487 | C | LEU | A | 721 | 77.460 | 79.459 | 64.588 | 1.00 | 28.32 | 6 |
| ATOM | 3488 | O | LEU | A | 721 | 76.278 | 79.624 | 64.892 | 1.00 | 32.28 | 8 |
| ATOM | 3489 | N | ARG | A | 722 | 78.012 | 80.023 | 63.515 | 1.00 | 28.59 | 7 |
| ATOM | 3490 | CA | ARG | A | 722 | 77.229 | 80.879 | 62.625 | 1.00 | 27.58 | 6 |
| ATOM | 3491 | CB | ARG | A | 722 | 78.148 | 81.738 | 61.742 | 1.00 | 26.26 | 6 |
| ATOM | 3492 | CG | ARG | A | 722 | 78.561 | 83.060 | 62.369 | 1.00 | 27.58 | 6 |
| ATOM | 3493 | CD | ARG | A | 722 | 79.504 | 83.858 | 61.467 | 1.00 | 26.00 | 6 |
| ATOM | 3494 | NE | ARG | A | 722 | 79.956 | 85.096 | 62.105 | 1.00 | 28.55 | 7 |
| ATOM | 3495 | CZ | ARG | A | 722 | 79.230 | 86.209 | 62.201 | 1.00 | 31.16 | 6 |
| ATOM | 3496 | NH1 | ARG | A | 722 | 78.006 | 86.258 | 61.692 | 1.00 | 32.64 | 7 |
| ATOM | 3497 | NH2 | ARG | A | 722 | 79.721 | 87.271 | 62.826 | 1.00 | 29.66 | 7 |
| ATOM | 3498 | C | ARG | A | 722 | 76.266 | 80.088 | 61.737 | 1.00 | 28.76 | 6 |
| ATOM | 3499 | O | ARG | A | 722 | 75.571 | 80.662 | 60.903 | 1.00 | 29.86 | 8 |
| ATOM | 3500 | N | GLY | A | 723 | 76.214 | 78.777 | 61.919 | 1.00 | 27.73 | 7 |
| ATOM | 3501 | CA | GLY | A | 723 | 75.316 | 77.972 | 61.116 | 1.00 | 27.44 | 6 |
| ATOM | 3502 | C | GLY | A | 723 | 74.527 | 76.896 | 61.846 | 1.00 | 26.60 | 6 |
| ATOM | 3503 | O | GLY | A | 723 | 73.674 | 76.258 | 61.240 | 1.00 | 26.55 | 8 |
| ATOM | 3504 | N | CYS | A | 724 | 74.782 | 76.684 | 63.136 | 1.00 | 26.94 | 7 |
| ATOM | 3505 | CA | CYS | A | 724 | 74.057 | 75.641 | 63.874 | 1.00 | 25.15 | 6 |
| ATOM | 3506 | CB | CYS | A | 724 | 74.781 | 75.307 | 65.190 | 1.00 | 25.35 | 6 |
| ATOM | 3507 | SG | CYS | A | 724 | 74.966 | 76.728 | 66.319 | 1.00 | 25.08 | 16 |
| ATOM | 3508 | C | CYS | A | 724 | 72.592 | 75.980 | 64.182 | 1.00 | 28.42 | 6 |
| ATOM | 3509 | O | CYS | A | 724 | 71.781 | 75.080 | 64.442 | 1.00 | 26.43 | 8 |
| ATOM | 3510 | N | GLY | A | 725 | 72.280 | 77.267 | 64.158 | 1.00 | 30.59 | 7 |
| ATOM | 3511 | CA | GLY | A | 725 | 70.938 | 77.795 | 64.374 | 1.00 | 32.14 | 6 |
| ATOM | 3512 | C | GLY | A | 725 | 70.704 | 78.337 | 65.794 | 1.00 | 32.94 | 6 |
| ATOM | 3513 | O | GLY | A | 725 | 71.508 | 78.102 | 66.690 | 1.00 | 33.45 | 8 |
| ATOM | 3514 | N | THR | A | 726 | 69.596 | 79.046 | 65.979 | 1.00 | 35.63 | 7 |
| ATOM | 3515 | CA | THR | A | 726 | 69.298 | 79.622 | 67.291 | 1.00 | 34.78 | 6 |
| ATOM | 3516 | CB | THR | A | 726 | 68.360 | 80.832 | 67.079 | 1.00 | 36.55 | 6 |
| ATOM | 3517 | OG1 | THR | A | 726 | 67.025 | 80.354 | 66.947 | 1.00 | 41.00 | 8 |
| ATOM | 3518 | CG2 | THR | A | 726 | 68.748 | 81.604 | 65.820 | 1.00 | 36.19 | 6 |
| ATOM | 3519 | C | THR | A | 726 | 68.808 | 78.595 | 68.356 | 1.00 | 34.05 | 6 |
| ATOM | 3520 | O | THR | A | 726 | 69.098 | 78.827 | 69.528 | 1.00 | 34.11 | 8 |
| ATOM | 3521 | N | ALA | A | 727 | 68.089 | 77.500 | 68.065 | 1.00 | 33.80 | 7 |
| ATOM | 3522 | CA | ALA | A | 727 | 67.670 | 76.533 | 69.143 | 1.00 | 34.58 | 6 |
| ATOM | 3523 | CB | ALA | A | 727 | 66.774 | 75.460 | 68.563 | 1.00 | 34.24 | 6 |
| ATOM | 3524 | C | ALA | A | 727 | 68.897 | 75.895 | 69.809 | 1.00 | 35.51 | 6 |
| ATOM | 3525 | O | ALA | A | 727 | 69.021 | 75.854 | 71.031 | 1.00 | 35.48 | 8 |
| ATOM | 3526 | N | MET | A | 728 | 69.784 | 75.410 | 68.943 | 1.00 | 35.24 | 7 |
| ATOM | 3527 | CA | MET | A | 728 | 71.044 | 74.844 | 69.365 | 1.00 | 33.77 | 6 |
| ATOM | 3528 | CB | MET | A | 728 | 71.876 | 74.327 | 68.186 | 1.00 | 36.98 | 6 |
| ATOM | 3529 | CG | MET | A | 728 | 71.256 | 73.155 | 67.465 | 1.00 | 40.03 | 6 |
| ATOM | 3530 | SD | MET | A | 728 | 71.719 | 71.586 | 68.229 | 1.00 | 47.90 | 16 |
| ATOM | 3531 | CE | MET | A | 728 | 73.415 | 71.445 | 67.688 | 1.00 | 44.52 | 6 |
| ATOM | 3532 | C | MET | A | 728 | 71.867 | 75.902 | 70.045 | 1.00 | 31.36 | 6 |
| ATOM | 3533 | O | MET | A | 728 | 72.348 | 75.740 | 71.156 | 1.00 | 27.87 | 8 |
| ATOM | 3534 | N | LEU | A | 729 | 72.012 | 76.983 | 69.330 | 1.00 | 30.10 | 7 |
| ATOM | 3535 | CA | LEU | A | 729 | 72.793 | 78.094 | 69.807 | 1.00 | 30.76 | 6 |
| ATOM | 3536 | CB | LEU | A | 729 | 72.430 | 79.336 | 69.044 | 1.00 | 31.61 | 6 |
| ATOM | 3537 | CG | LEU | A | 729 | 73.206 | 79.536 | 67.752 | 1.00 | 34.19 | 6 |
| ATOM | 3538 | CD1 | LEU | A | 729 | 72.832 | 80.852 | 67.093 | 1.00 | 36.13 | 6 |
| ATOM | 3539 | CD2 | LEU | A | 729 | 74.703 | 79.488 | 68.008 | 1.00 | 34.00 | 6 |
| ATOM | 3540 | C | LEU | A | 729 | 72.609 | 78.334 | 71.258 | 1.00 | 31.63 | 6 |
| ATOM | 3541 | O | LEU | A | 729 | 73.521 | 78.786 | 71.936 | 1.00 | 31.40 | 8 |
| ATOM | 3542 | N | HIS | A | 730 | 71.448 | 78.062 | 71.739 | 1.00 | 32.99 | 7 |
| ATOM | 3543 | CA | HIS | A | 730 | 71.350 | 78.350 | 73.121 | 1.00 | 36.17 | 6 |
| ATOM | 3544 | CB | HIS | A | 730 | 70.134 | 79.187 | 73.333 | 1.00 | 40.54 | 6 |
| ATOM | 3545 | CG | HIS | A | 730 | 69.213 | 78.929 | 74.526 | 1.00 | 44.11 | 6 |
| ATOM | 3546 | CD2 | HIS | A | 730 | 67.867 | 78.736 | 74.485 | 1.00 | 44.29 | 6 |
| ATOM | 3547 | ND1 | HIS | A | 730 | 69.591 | 78.913 | 75.862 | 1.00 | 45.50 | 7 |
| ATOM | 3548 | CE1 | HIS | A | 730 | 68.501 | 78.745 | 76.594 | 1.00 | 44.90 | 6 |
| ATOM | 3549 | NE2 | HIS | A | 730 | 67.460 | 78.643 | 75.784 | 1.00 | 46.30 | 7 |
| ATOM | 3550 | C | HIS | A | 730 | 71.506 | 77.074 | 73.913 | 1.00 | 33.74 | 6 |
| ATOM | 3551 | O | HIS | A | 730 | 72.382 | 77.001 | 74.773 | 1.00 | 30.61 | 8 |
| ATOM | 3552 | N | ASP | A | 731 | 70.696 | 76.087 | 73.656 | 1.00 | 32.97 | 7 |
| ATOM | 3553 | CA | ASP | A | 731 | 70.998 | 74.919 | 74.432 | 1.00 | 33.98 | 6 |
| ATOM | 3554 | CB | ASP | A | 731 | 70.802 | 73.622 | 73.657 | 1.00 | 37.99 | 6 |
| ATOM | 3555 | CG | ASP | A | 731 | 69.355 | 73.357 | 73.402 | 1.00 | 42.51 | 6 |
| ATOM | 3556 | OD1 | ASP | A | 731 | 68.502 | 73.695 | 74.257 | 1.00 | 44.05 | 8 |
| ATOM | 3557 | OD2 | ASP | A | 731 | 69.041 | 72.796 | 72.327 | 1.00 | 43.97 | 8 |
| ATOM | 3558 | C | ASP | A | 731 | 72.464 | 75.057 | 74.805 | 1.00 | 32.49 | 6 |
| ATOM | 3559 | O | ASP | A | 731 | 72.864 | 74.787 | 75.951 | 1.00 | 33.78 | 8 |
| ATOM | 3560 | N | PHE | A | 732 | 73.282 | 75.499 | 73.841 | 1.00 | 29.82 | 7 |
| ATOM | 3561 | CA | PHE | A | 732 | 74.698 | 75.633 | 74.134 | 1.00 | 27.64 | 6 |
| ATOM | 3562 | CB | PHE | A | 732 | 75.540 | 76.041 | 72.918 | 1.00 | 27.65 | 6 |
| ATOM | 3563 | CG | PHE | A | 732 | 75.895 | 74.926 | 71.967 | 1.00 | 25.81 | 6 |
| ATOM | 3564 | CD1 | PHE | A | 732 | 75.759 | 75.149 | 70.599 | 1.00 | 23.08 | 6 |
| ATOM | 3565 | CD2 | PHE | A | 732 | 76.319 | 73.672 | 72.398 | 1.00 | 20.52 | 6 |
| ATOM | 3566 | CE1 | PHE | A | 732 | 76.070 | 74.145 | 69.676 | 1.00 | 23.70 | 6 |
| ATOM | 3567 | CE2 | PHE | A | 732 | 76.604 | 72.659 | 71.481 | 1.00 | 23.04 | 6 |
| ATOM | 3568 | CZ | PHE | A | 732 | 76.496 | 72.895 | 70.116 | 1.00 | 23.57 | 6 |
| ATOM | 3569 | C | PHE | A | 732 | 74.971 | 76.680 | 75.179 | 1.00 | 28.01 | 6 |
| ATOM | 3570 | O | PHE | A | 732 | 75.715 | 76.429 | 76.132 | 1.00 | 24.31 | 8 |
| ATOM | 3571 | N | THR | A | 733 | 74.413 | 77.853 | 75.046 | 1.00 | 29.61 | 7 |
| ATOM | 3572 | CA | THR | A | 733 | 74.775 | 78.815 | 76.060 | 1.00 | 29.51 | 6 |
| ATOM | 3573 | CB | THR | A | 733 | 74.297 | 80.186 | 75.594 | 1.00 | 31.32 | 6 |
| ATOM | 3574 | OG1 | THR | A | 733 | 74.473 | 80.288 | 74.174 | 1.00 | 29.17 | 8 |
| ATOM | 3575 | CG2 | THR | A | 733 | 75.088 | 81.270 | 76.304 | 1.00 | 30.83 | 6 |
| ATOM | 3576 | C | THR | A | 733 | 74.309 | 78.341 | 77.445 | 1.00 | 29.80 | 6 |
| ATOM | 3577 | O | THR | A | 733 | 75.052 | 78.499 | 78.432 | 1.00 | 30.30 | 8 |
| ATOM | 3578 | N | GLN | A | 734 | 73.101 | 77.774 | 77.551 | 1.00 | 29.60 | 7 |
| ATOM | 3579 | CA | GLN | A | 734 | 72.602 | 77.296 | 78.861 | 1.00 | 32.46 | 6 |
| ATOM | 3580 | CB | GLN | A | 734 | 71.301 | 76.495 | 78.724 | 1.00 | 35.93 | 6 |
| ATOM | 3581 | CG | GLN | A | 734 | 70.018 | 77.325 | 78.527 | 1.00 | 41.57 | 6 |
| ATOM | 3582 | CD | GLN | A | 734 | 69.060 | 77.246 | 79.720 | 1.00 | 45.73 | 6 |
| ATOM | 3583 | OE1 | GLN | A | 734 | 68.268 | 76.307 | 79.847 | 1.00 | 48.38 | 8 |
| ATOM | 3584 | NE2 | GLN | A | 734 | 68.962 | 78.132 | 80.718 | 1.00 | 48.03 | 7 |
| ATOM | 3585 | C | GLN | A | 734 | 73.670 | 76.433 | 79.474 | 1.00 | 32.05 | 6 |
| ATOM | 3586 | O | GLN | A | 734 | 74.040 | 76.536 | 80.634 | 1.00 | 30.04 | 8 |
| ATOM | 3587 | N | GLN | A | 735 | 74.148 | 75.564 | 78.612 | 1.00 | 30.98 | 7 |
| ATOM | 3588 | CA | GLN | A | 735 | 75.195 | 74.604 | 78.921 | 1.00 | 30.15 | 6 |
| ATOM | 3589 | CB | GLN | A | 735 | 75.604 | 73.827 | 77.685 | 1.00 | 30.80 | 6 |
| ATOM | 3590 | CG | GLN | A | 735 | 75.076 | 72.412 | 77.591 | 1.00 | 28.02 | 6 |
| ATOM | 3591 | CD | GLN | A | 735 | 75.381 | 71.843 | 76.237 | 1.00 | 29.18 | 6 |
| ATOM | 3592 | OE1 | GLN | A | 735 | 76.546 | 71.652 | 75.864 | 1.00 | 27.87 | 8 |
| ATOM | 3593 | NE2 | GLN | A | 735 | 74.475 | 71.503 | 75.323 | 1.00 | 29.11 | 7 |
| ATOM | 3594 | C | GLN | A | 735 | 76.464 | 75.283 | 79.433 | 1.00 | 29.53 | 6 |
| ATOM | 3595 | O | GLN | A | 735 | 76.971 | 75.017 | 80.530 | 1.00 | 28.10 | 8 |
| ATOM | 3596 | N | VAL | A | 736 | 76.959 | 76.163 | 78.576 | 1.00 | 29.43 | 7 |
| ATOM | 3597 | CA | VAL | A | 736 | 78.175 | 76.897 | 78.898 | 1.00 | 30.13 | 6 |
| ATOM | 3598 | CB | VAL | A | 736 | 78.580 | 77.832 | 77.744 | 1.00 | 29.01 | 6 |
| ATOM | 3599 | CG1 | VAL | A | 736 | 79.737 | 78.726 | 78.173 | 1.00 | 31.28 | 6 |
| ATOM | 3600 | CG2 | VAL | A | 736 | 78.941 | 77.027 | 76.507 | 1.00 | 30.67 | 6 |
| ATOM | 3601 | C | VAL | A | 736 | 78.019 | 77.686 | 80.205 | 1.00 | 30.77 | 6 |
| ATOM | 3602 | O | VAL | A | 736 | 78.948 | 77.668 | 81.034 | 1.00 | 31.00 | 8 |
| ATOM | 3603 | N | GLN | A | 737 | 76.901 | 78.367 | 80.432 | 1.00 | 31.96 | 7 |
| ATOM | 3604 | CA | GLN | A | 737 | 76.740 | 79.128 | 81.666 | 1.00 | 34.69 | 6 |
| ATOM | 3605 | CB | GLN | A | 737 | 75.407 | 79.874 | 81.670 | 1.00 | 37.40 | 6 |
| ATOM | 3606 | CG | GLN | A | 737 | 75.222 | 80.771 | 80.475 | 1.00 | 41.15 | 6 |
| ATOM | 3607 | CD | GLN | A | 737 | 73.963 | 81.586 | 80.563 | 1.00 | 42.60 | 6 |
| ATOM | 3608 | OE1 | GLN | A | 737 | 72.937 | 81.110 | 81.048 | 1.00 | 44.03 | 8 |
| ATOM | 3609 | NE2 | GLN | A | 737 | 73.807 | 82.853 | 80.173 | 1.00 | 45.10 | 7 |
| ATOM | 3610 | C | GLN | A | 737 | 76.804 | 78.194 | 82.865 | 1.00 | 33.27 | 6 |
| ATOM | 3611 | O | GLN | A | 737 | 77.386 | 78.534 | 83.891 | 1.00 | 33.38 | 8 |
| ATOM | 3612 | N | VAL | A | 738 | 76.209 | 77.015 | 82.734 | 1.00 | 33.58 | 7 |
| ATOM | 3613 | CA | VAL | A | 738 | 76.235 | 76.053 | 83.822 | 1.00 | 33.76 | 6 |
| ATOM | 3614 | CB | VAL | A | 738 | 75.311 | 74.843 | 83.531 | 1.00 | 35.79 | 6 |
| ATOM | 3615 | CG1 | VAL | A | 738 | 75.568 | 73.707 | 84.514 | 1.00 | 34.22 | 6 |
| ATOM | 3616 | CG2 | VAL | A | 738 | 73.847 | 75.269 | 83.589 | 1.00 | 33.91 | 6 |
| ATOM | 3617 | C | VAL | A | 738 | 77.639 | 75.541 | 84.129 | 1.00 | 34.42 | 6 |
| ATOM | 3618 | O | VAL | A | 738 | 78.008 | 75.522 | 85.300 | 1.00 | 33.60 | 8 |
| ATOM | 3619 | N | ILE | A | 739 | 78.426 | 75.115 | 83.141 | 1.00 | 33.03 | 7 |
| ATOM | 3620 | CA | ILE | A | 739 | 79.759 | 74.659 | 83.454 | 1.00 | 34.41 | 6 |
| ATOM | 3621 | CB | ILE | A | 739 | 80.569 | 74.188 | 82.216 | 1.00 | 34.02 | 6 |
| ATOM | 3622 | CG2 | ILE | A | 739 | 79.780 | 73.143 | 81.445 | 1.00 | 33.45 | 6 |
| ATOM | 3623 | CG1 | ILE | A | 739 | 80.923 | 75.352 | 81.286 | 1.00 | 37.98 | 6 |
| ATOM | 3624 | CD1 | ILE | A | 739 | 82.181 | 75.124 | 80.475 | 1.00 | 37.56 | 6 |
| ATOM | 3625 | C | ILE | A | 739 | 80.526 | 75.790 | 84.053 | 1.00 | 34.92 | 6 |
| ATOM | 3626 | O | ILE | A | 739 | 80.913 | 75.777 | 85.220 | 1.00 | 33.63 | 8 |
| ATOM | 3627 | N | GLU | A | 740 | 80.742 | 76.733 | 83.214 | 1.00 | 35.23 | 7 |
| ATOM | 3628 | CA | GLU | A | 740 | 81.443 | 77.896 | 83.616 | 1.00 | 38.19 | 6 |
| ATOM | 3629 | CB | GLU | A | 740 | 80.982 | 79.096 | 82.819 | 1.00 | 41.65 | 6 |
| ATOM | 3630 | CG | GLU | A | 740 | 82.123 | 80.009 | 82.462 | 1.00 | 46.36 | 6 |
| ATOM | 3631 | CD | GLU | A | 740 | 82.471 | 79.918 | 80.982 | 1.00 | 49.35 | 6 |
| ATOM | 3632 | OE1 | GLU | A | 740 | 81.714 | 80.473 | 80.150 | 1.00 | 49.25 | 8 |
| ATOM | 3633 | OE2 | GLU | A | 740 | 83.493 | 79.287 | 80.649 | 1.00 | 49.69 | 8 |
| ATOM | 3634 | C | GLU | A | 740 | 81.254 | 78.116 | 85.124 | 1.00 | 37.46 | 6 |
| ATOM | 3635 | O | GLU | A | 740 | 82.210 | 78.309 | 85.863 | 1.00 | 37.23 | 8 |
| ATOM | 3636 | N | MET | A | 741 | 79.997 | 78.072 | 85.538 | 1.00 | 38.07 | 7 |
| ATOM | 3637 | CA | MET | A | 741 | 79.594 | 78.250 | 86.949 | 1.00 | 37.88 | 6 |
| ATOM | 3638 | CB | MET | A | 741 | 78.077 | 78.137 | 87.077 | 1.00 | 40.77 | 6 |
| ATOM | 3639 | CG | MET | A | 741 | 77.404 | 79.455 | 87.402 | 1.00 | 47.17 | 6 |
| ATOM | 3640 | SD | MET | A | 741 | 75.604 | 79.335 | 87.408 | 1.00 | 53.04 | 16 |
| ATOM | 3641 | CE | MET | A | 741 | 75.183 | 80.691 | 88.498 | 1.00 | 53.54 | 6 |
| ATOM | 3642 | C | MET | A | 741 | 80.237 | 77.228 | 87.855 | 1.00 | 37.06 | 6 |
| ATOM | 3643 | O | MET | A | 741 | 80.919 | 77.541 | 88.833 | 1.00 | 35.78 | 8 |
| ATOM | 3644 | N | LEU | A | 742 | 79.990 | 75.966 | 87.493 | 1.00 | 34.36 | 7 |
| ATOM | 3645 | CA | LEU | A | 742 | 80.489 | 74.811 | 88.245 | 1.00 | 34.69 | 6 |
| ATOM | 3646 | CB | LEU | A | 742 | 79.816 | 73.531 | 87.765 | 1.00 | 35.38 | 6 |
| ATOM | 3647 | CG | LEU | A | 742 | 78.296 | 73.538 | 87.925 | 1.00 | 36.62 | 6 |
| ATOM | 3648 | CD1 | LEU | A | 742 | 77.680 | 72.297 | 87.299 | 1.00 | 36.03 | 6 |
| ATOM | 3649 | CD2 | LEU | A | 742 | 77.909 | 73.635 | 89.379 | 1.00 | 35.59 | 6 |
| ATOM | 3650 | C | LEU | A | 742 | 82.005 | 74.725 | 88.173 | 1.00 | 33.04 | 6 |
| ATOM | 3651 | O | LEU | A | 742 | 82.631 | 74.158 | 89.063 | 1.00 | 32.97 | 8 |
| ATOM | 3652 | N | GLN | A | 743 | 82.599 | 75.281 | 87.140 | 1.00 | 35.55 | 7 |
| ATOM | 3653 | CA | GLN | A | 743 | 84.051 | 75.248 | 87.007 | 1.00 | 35.35 | 6 |
| ATOM | 3654 | CB | GLN | A | 743 | 84.501 | 75.753 | 85.609 | 1.00 | 37.19 | 6 |
| ATOM | 3655 | CG | GLN | A | 743 | 84.510 | 74.679 | 84.512 | 1.00 | 41.76 | 6 |
| ATOM | 3656 | CD | GLN | A | 743 | 85.140 | 75.117 | 83.182 | 1.00 | 42.43 | 6 |
| ATOM | 3657 | OE1 | GLN | A | 743 | 84.853 | 76.204 | 82.677 | 1.00 | 46.38 | 8 |
| ATOM | 3658 | NE2 | GLN | A | 743 | 86.002 | 74.418 | 82.439 | 1.00 | 43.07 | 7 |
| ATOM | 3659 | C | GLN | A | 743 | 84.716 | 76.020 | 88.170 | 1.00 | 35.52 | 6 |
| ATOM | 3660 | O | GLN | A | 743 | 85.576 | 75.468 | 88.863 | 1.00 | 32.88 | 8 |
| ATOM | 3661 | N | LYS | A | 744 | 84.300 | 77.264 | 88.382 | 1.00 | 34.93 | 7 |
| ATOM | 3662 | CA | LYS | A | 744 | 84.768 | 78.096 | 89.477 | 1.00 | 36.49 | 6 |
| ATOM | 3663 | CB | LYS | A | 744 | 83.907 | 79.366 | 89.552 | 1.00 | 37.59 | 6 |
| ATOM | 3664 | CG | LYS | A | 744 | 84.526 | 80.605 | 90.201 | 1.00 | 41.70 | 6 |
| ATOM | 3665 | CD | LYS | A | 744 | 83.457 | 81.596 | 90.632 | 1.00 | 45.14 | 6 |
| ATOM | 3666 | CE | LYS | A | 744 | 83.830 | 83.038 | 90.305 | 1.00 | 46.60 | 6 |
| ATOM | 3667 | NZ | LYS | A | 744 | 82.828 | 84.006 | 90.826 | 1.00 | 46.89 | 7 |
| ATOM | 3668 | C | LYS | A | 744 | 84.678 | 77.316 | 90.792 | 1.00 | 34.45 | 6 |
| ATOM | 3669 | O | LYS | A | 744 | 85.678 | 76.930 | 91.383 | 1.00 | 34.90 | 8 |
| ATOM | 3670 | N | VAL | A | 745 | 83.417 | 77.109 | 91.237 | 1.00 | 34.37 | 7 |
| ATOM | 3671 | CA | VAL | A | 745 | 83.113 | 76.388 | 92.482 | 1.00 | 33.97 | 6 |
| ATOM | 3672 | CB | VAL | A | 745 | 81.692 | 75.817 | 92.443 | 1.00 | 35.32 | 6 |
| ATOM | 3673 | CG1 | VAL | A | 745 | 81.380 | 75.084 | 93.733 | 1.00 | 36.09 | 6 |
| ATOM | 3674 | CG2 | VAL | A | 745 | 80.670 | 76.920 | 92.206 | 1.00 | 35.76 | 6 |
| ATOM | 3675 | C | VAL | A | 745 | 84.101 | 75.284 | 92.663 | 1.00 | 34.87 | 6 |
| ATOM | 3676 | O | VAL | A | 745 | 84.687 | 75.124 | 93.732 | 1.00 | 34.85 | 8 |
| ATOM | 3677 | N | THR | A | 746 | 84.252 | 74.546 | 91.618 | 1.00 | 33.15 | 7 |
| ATOM | 3678 | CA | THR | A | 746 | 85.214 | 73.528 | 91.630 | 1.00 | 31.91 | 6 |
| ATOM | 3679 | CB | THR | A | 746 | 85.376 | 72.945 | 90.254 | 1.00 | 30.38 | 6 |
| ATOM | 3680 | OG1 | THR | A | 746 | 84.115 | 72.898 | 89.583 | 1.00 | 29.21 | 8 |
| ATOM | 3681 | CG2 | THR | A | 746 | 85.964 | 71.542 | 90.345 | 1.00 | 29.71 | 6 |
| ATOM | 3682 | C | THR | A | 746 | 86.527 | 74.103 | 92.141 | 1.00 | 33.46 | 6 |
| ATOM | 3683 | O | THR | A | 746 | 86.889 | 73.823 | 93.278 | 1.00 | 30.24 | 8 |
| ATOM | 3684 | N | LEU | A | 747 | 87.244 | 74.908 | 91.362 | 1.00 | 35.48 | 7 |
| ATOM | 3685 | CA | LEU | A | 747 | 88.528 | 75.424 | 91.844 | 1.00 | 38.82 | 6 |
| ATOM | 3686 | CB | LEU | A | 747 | 89.133 | 76.311 | 90.772 | 1.00 | 38.90 | 6 |
| ATOM | 3687 | CG | LEU | A | 747 | 89.717 | 75.531 | 89.587 | 1.00 | 40.10 | 6 |
| ATOM | 3688 | CD1 | LEU | A | 747 | 90.180 | 76.488 | 88.500 | 1.00 | 37.87 | 6 |
| ATOM | 3689 | CD2 | LEU | A | 747 | 90.848 | 74.632 | 90.029 | 1.00 | 38.63 | 6 |
| ATOM | 3690 | C | LEU | A | 747 | 88.536 | 76.145 | 93.164 | 1.00 | 40.05 | 6 |
| ATOM | 3691 | O | LEU | A | 747 | 89.503 | 76.068 | 93.903 | 1.00 | 41.90 | 8 |
| ATOM | 3692 | N | ASP | A | 748 | 87.475 | 76.838 | 93.482 | 1.00 | 42.06 | 7 |
| ATOM | 3693 | CA | ASP | A | 748 | 87.478 | 77.568 | 94.750 | 1.00 | 42.62 | 6 |
| ATOM | 3694 | CB | ASP | A | 748 | 86.179 | 78.365 | 94.884 | 1.00 | 43.08 | 6 |
| ATOM | 3695 | CG | ASP | A | 748 | 86.365 | 79.832 | 94.544 | 1.00 | 43.20 | 6 |
| ATOM | 3696 | OD1 | ASP | A | 748 | 87.516 | 80.290 | 94.478 | 1.00 | 43.54 | 8 |
| ATOM | 3697 | OD2 | ASP | A | 748 | 85.343 | 80.519 | 94.344 | 1.00 | 45.60 | 8 |
| ATOM | 3698 | C | ASP | A | 748 | 87.602 | 76.646 | 95.975 | 1.00 | 44.30 | 6 |
| ATOM | 3699 | O | ASP | A | 748 | 88.495 | 76.793 | 96.794 | 1.00 | 44.64 | 8 |
| ATOM | 3700 | N | ILE | A | 749 | 86.649 | 75.693 | 96.068 | 1.00 | 44.84 | 7 |
| ATOM | 3701 | CA | ILE | A | 749 | 86.601 | 74.714 | 97.146 | 1.00 | 45.33 | 6 |
| ATOM | 3702 | CB | ILE | A | 749 | 85.620 | 73.542 | 96.850 | 1.00 | 45.81 | 6 |
| ATOM | 3703 | CG2 | ILE | A | 749 | 86.193 | 72.210 | 97.313 | 1.00 | 45.18 | 6 |
| ATOM | 3704 | CG1 | ILE | A | 749 | 84.268 | 73.815 | 97.512 | 1.00 | 46.09 | 6 |
| ATOM | 3705 | CD1 | ILE | A | 749 | 83.748 | 75.219 | 97.295 | 1.00 | 47.83 | 6 |
| ATOM | 3706 | C | ILE | A | 749 | 88.009 | 74.229 | 97.339 | 1.00 | 46.11 | 6 |
| ATOM | 3707 | O | ILE | A | 749 | 88.375 | 73.591 | 98.322 | 1.00 | 45.22 | 8 |
| ATOM | 3708 | N | LYS | A | 750 | 88.799 | 74.581 | 96.340 | 1.00 | 48.07 | 7 |
| ATOM | 3709 | CA | LYS | A | 750 | 90.209 | 74.293 | 96.414 | 1.00 | 50.37 | 6 |
| ATOM | 3710 | CB | LYS | A | 750 | 90.817 | 73.923 | 95.033 | 1.00 | 50.55 | 6 |
| ATOM | 3711 | CG | LYS | A | 750 | 92.140 | 73.146 | 95.129 | 1.00 | 51.49 | 6 |
| ATOM | 3712 | CD | LYS | A | 750 | 92.325 | 72.097 | 94.044 | 1.00 | 53.90 | 6 |
| ATOM | 3713 | CE | LYS | A | 750 | 93.669 | 71.392 | 94.178 | 1.00 | 53.54 | 6 |
| ATOM | 3714 | NZ | LYS | A | 750 | 94.100 | 70.788 | 92.890 | 1.00 | 54.24 | 7 |
| ATOM | 3715 | C | LYS | A | 750 | 90.908 | 75.498 | 97.047 | 1.00 | 51.73 | 6 |
| ATOM | 3716 | O | LYS | A | 750 | 91.640 | 75.349 | 98.021 | 1.00 | 52.33 | 8 |
| ATOM | 3717 | N | SER | A | 751 | 90.678 | 76.691 | 96.524 | 1.00 | 52.79 | 7 |
| ATOM | 3718 | CA | SER | A | 751 | 91.319 | 77.850 | 97.125 | 1.00 | 54.48 | 6 |
| ATOM | 3719 | CB | SER | A | 751 | 90.568 | 79.138 | 96.844 | 1.00 | 52.83 | 6 |
| ATOM | 3720 | OG | SER | A | 751 | 91.002 | 79.741 | 95.631 | 1.00 | 53.21 | 8 |
| ATOM | 3721 | C | SER | A | 751 | 91.384 | 77.567 | 98.636 | 1.00 | 55.62 | 6 |
| ATOM | 3722 | O | SER | A | 751 | 91.181 | 78.451 | 99.460 | 1.00 | 57.35 | 8 |
| ATOM | 3723 | N | LEU | A | 752 | 91.686 | 76.298 | 99.003 | 1.00 | 56.26 | 7 |
| ATOM | 3724 | CA | LEU | A | 752 | 91.623 | 75.939 | 100.438 | 1.00 | 58.33 | 6 |
| ATOM | 3725 | CB | LEU | A | 752 | 90.242 | 75.303 | 100.728 | 1.00 | 56.15 | 6 |
| ATOM | 3726 | CG | LEU | A | 752 | 88.980 | 76.174 | 100.841 | 1.00 | 55.46 | 6 |
| ATOM | 3727 | CD1 | LEU | A | 752 | 87.912 | 75.450 | 101.645 | 1.00 | 54.86 | 6 |
| ATOM | 3728 | CD2 | LEU | A | 752 | 89.303 | 77.525 | 101.468 | 1.00 | 54.25 | 6 |
| ATOM | 3729 | C | LEU | A | 752 | 92.607 | 74.929 | 101.030 | 1.00 | 60.54 | 6 |
| ATOM | 3730 | O | LEU | A | 752 | 93.694 | 75.248 | 101.481 | 1.00 | 60.83 | 8 |
| ATOM | 3731 | N | SER | A | 753 | 92.144 | 73.692 | 100.983 | 1.00 | 63.74 | 7 |
| ATOM | 3732 | CA | SER | A | 753 | 92.739 | 72.472 | 101.563 | 1.00 | 67.43 | 6 |
| ATOM | 3733 | CB | SER | A | 753 | 91.655 | 71.397 | 101.513 | 1.00 | 67.80 | 6 |
| ATOM | 3734 | OG | SER | A | 753 | 90.391 | 71.951 | 101.180 | 1.00 | 68.98 | 8 |
| ATOM | 3735 | C | SER | A | 753 | 94.061 | 71.916 | 100.996 | 1.00 | 69.79 | 6 |
| ATOM | 3736 | O | SER | A | 753 | 95.109 | 72.132 | 101.604 | 1.00 | 70.29 | 8 |
| ATOM | 3737 | N | SER | A | 760 | 86.519 | 67.703 | 105.201 | 1.00 | 46.48 | 7 |
| ATOM | 3738 | CA | SER | A | 760 | 85.862 | 67.983 | 106.463 | 1.00 | 44.10 | 6 |
| ATOM | 3739 | CB | SER | A | 760 | 86.630 | 69.064 | 107.224 | 1.00 | 44.56 | 6 |
| ATOM | 3740 | OG | SER | A | 760 | 86.562 | 70.297 | 106.528 | 1.00 | 42.23 | 8 |
| ATOM | 3741 | C | SER | A | 760 | 84.459 | 68.487 | 106.182 | 1.00 | 41.64 | 6 |
| ATOM | 3742 | O | SER | A | 760 | 84.131 | 68.825 | 105.048 | 1.00 | 40.47 | 8 |
| ATOM | 3743 | N | SER | A | 761 | 83.636 | 68.535 | 107.222 | 1.00 | 40.81 | 7 |
| ATOM | 3744 | CA | SER | A | 761 | 82.278 | 69.029 | 107.083 | 1.00 | 40.39 | 6 |
| ATOM | 3745 | CB | SER | A | 761 | 81.513 | 68.854 | 108.397 | 1.00 | 42.54 | 6 |
| ATOM | 3746 | OG | SER | A | 761 | 82.158 | 69.538 | 109.456 | 1.00 | 44.07 | 8 |
| ATOM | 3747 | C | SER | A | 761 | 82.375 | 70.504 | 106.715 | 1.00 | 38.07 | 6 |
| ATOM | 3748 | O | SER | A | 761 | 81.432 | 71.081 | 106.179 | 1.00 | 38.40 | 8 |
| ATOM | 3749 | N | GLN | A | 762 | 83.534 | 71.096 | 107.000 | 1.00 | 36.50 | 7 |
| ATOM | 3750 | CA | GLN | A | 762 | 83.789 | 72.502 | 106.696 | 1.00 | 35.16 | 6 |
| ATOM | 3751 | CB | GLN | A | 762 | 85.141 | 72.939 | 107.281 | 1.00 | 34.85 | 6 |
| ATOM | 3752 | CG | GLN | A | 762 | 85.527 | 74.382 | 106.971 | 1.00 | 37.14 | 6 |
| ATOM | 3753 | CD | GLN | A | 762 | 86.845 | 74.790 | 107.619 | 1.00 | 37.45 | 6 |
| ATOM | 3754 | OE1 | GLN | A | 762 | 86.957 | 74.857 | 108.841 | 1.00 | 34.83 | 8 |
| ATOM | 3755 | NE2 | GLN | A | 762 | 87.850 | 75.057 | 106.796 | 1.00 | 40.74 | 7 |
| ATOM | 3756 | C | GLN | A | 762 | 83.785 | 72.755 | 105.186 | 1.00 | 33.53 | 6 |
| ATOM | 3757 | O | GLN | A | 762 | 83.036 | 73.602 | 104.698 | 1.00 | 31.40 | 8 |
| ATOM | 3758 | N | VAL | A | 763 | 84.618 | 72.024 | 104.451 | 1.00 | 34.48 | 7 |
| ATOM | 3759 | CA | VAL | A | 763 | 84.685 | 72.204 | 103.003 | 1.00 | 35.27 | 6 |
| ATOM | 3760 | CB | VAL | A | 763 | 85.835 | 71.378 | 102.368 | 1.00 | 36.65 | 6 |
| ATOM | 3761 | CG1 | VAL | A | 763 | 85.578 | 69.888 | 102.534 | 1.00 | 37.55 | 6 |
| ATOM | 3762 | CG2 | VAL | A | 763 | 85.970 | 71.735 | 100.892 | 1.00 | 35.79 | 6 |
| ATOM | 3763 | C | VAL | A | 763 | 83.367 | 71.813 | 102.341 | 1.00 | 35.57 | 6 |
| ATOM | 3764 | O | VAL | A | 763 | 82.966 | 72.408 | 101.342 | 1.00 | 35.71 | 8 |
| ATOM | 3765 | N | ILE | A | 764 | 82.688 | 70.813 | 102.891 | 1.00 | 36.41 | 7 |
| ATOM | 3766 | CA | ILE | A | 764 | 81.415 | 70.411 | 102.310 | 1.00 | 38.31 | 6 |
| ATOM | 3767 | CB | ILE | A | 764 | 80.910 | 69.077 | 102.894 | 1.00 | 38.35 | 6 |
| ATOM | 3768 | CG2 | ILE | A | 764 | 79.478 | 68.813 | 102.432 | 1.00 | 39.79 | 6 |
| ATOM | 3769 | CG1 | ILE | A | 764 | 81.835 | 67.938 | 102.437 | 1.00 | 40.32 | 6 |
| ATOM | 3770 | CD1 | ILE | A | 764 | 81.384 | 66.538 | 102.851 | 1.00 | 39.50 | 6 |
| ATOM | 3771 | C | ILE | A | 764 | 80.407 | 71.526 | 102.562 | 1.00 | 38.85 | 6 |
| ATOM | 3772 | O | ILE | A | 764 | 79.627 | 71.880 | 101.671 | 1.00 | 39.16 | 8 |
| ATOM | 3773 | N | SER | A | 765 | 80.440 | 72.094 | 103.765 | 1.00 | 38.04 | 7 |
| ATOM | 3774 | CA | SER | A | 765 | 79.540 | 73.190 | 104.108 | 1.00 | 36.94 | 6 |
| ATOM | 3775 | CB | SER | A | 765 | 79.800 | 73.670 | 105.541 | 1.00 | 37.54 | 6 |
| ATOM | 3776 | OG | SER | A | 765 | 79.144 | 74.904 | 105.809 | 1.00 | 34.49 | 8 |
| ATOM | 3777 | C | SER | A | 765 | 79.780 | 74.339 | 103.133 | 1.00 | 36.35 | 6 |
| ATOM | 3778 | O | SER | A | 765 | 78.842 | 74.960 | 102.640 | 1.00 | 37.12 | 8 |
| ATOM | 3779 | N | GLN | A | 766 | 81.050 | 74.613 | 102.857 | 1.00 | 36.40 | 7 |
| ATOM | 3780 | CA | GLN | A | 766 | 81.422 | 75.679 | 101.942 | 1.00 | 36.55 | 6 |
| ATOM | 3781 | CB | GLN | A | 766 | 82.939 | 75.871 | 101.965 | 1.00 | 39.15 | 6 |
| ATOM | 3782 | CG | GLN | A | 766 | 83.476 | 76.440 | 103.273 | 1.00 | 42.58 | 6 |
| ATOM | 3783 | CD | GLN | A | 766 | 83.090 | 77.900 | 103.496 | 1.00 | 46.15 | 6 |
| ATOM | 3784 | OE1 | GLN | A | 766 | 83.349 | 78.468 | 104.560 | 1.00 | 48.85 | 8 |
| ATOM | 3785 | NE2 | GLN | A | 766 | 82.475 | 78.517 | 102.489 | 1.00 | 49.82 | 7 |
| ATOM | 3786 | C | GLN | A | 766 | 80.951 | 75.366 | 100.520 | 1.00 | 36.58 | 6 |
| ATOM | 3787 | O | GLN | A | 766 | 80.649 | 76.272 | 99.742 | 1.00 | 34.87 | 8 |
| ATOM | 3788 | N | LEU | A | 767 | 80.899 | 74.082 | 100.182 | 1.00 | 35.53 | 7 |
| ATOM | 3789 | CA | LEU | A | 767 | 80.448 | 73.669 | 98.854 | 1.00 | 34.74 | 6 |
| ATOM | 3790 | CB | LEU | A | 767 | 80.677 | 72.166 | 98.630 | 1.00 | 32.27 | 6 |
| ATOM | 3791 | CG | LEU | A | 767 | 80.722 | 71.622 | 97.186 | 1.00 | 33.66 | 6 |
| ATOM | 3792 | CD1 | LEU | A | 767 | 80.246 | 70.169 | 97.185 | 1.00 | 32.28 | 6 |
| ATOM | 3793 | CD2 | LEU | A | 767 | 79.860 | 72.439 | 96.255 | 1.00 | 29.50 | 6 |
| ATOM | 3794 | C | LEU | A | 767 | 78.954 | 73.941 | 98.792 | 1.00 | 33.10 | 6 |
| ATOM | 3795 | O | LEU | A | 767 | 78.466 | 74.610 | 97.886 | 1.00 | 32.37 | 8 |
| ATOM | 3796 | N | LYS | A | 768 | 78.237 | 73.423 | 99.781 | 1.00 | 35.38 | 7 |
| ATOM | 3797 | CA | LYS | A | 768 | 76.796 | 73.574 | 99.841 | 1.00 | 37.51 | 6 |
| ATOM | 3798 | CB | LYS | A | 768 | 76.270 | 72.906 | 101.106 | 1.00 | 40.21 | 6 |
| ATOM | 3799 | CG | LYS | A | 768 | 74.892 | 72.316 | 100.947 | 1.00 | 43.48 | 6 |
| ATOM | 3800 | CD | LYS | A | 768 | 74.724 | 71.052 | 101.782 | 1.00 | 45.10 | 6 |
| ATOM | 3801 | CE | LYS | A | 768 | 75.717 | 69.977 | 101.367 | 1.00 | 44.44 | 6 |
| ATOM | 3802 | NZ | LYS | A | 768 | 75.363 | 68.651 | 101.936 | 1.00 | 45.41 | 7 |
| ATOM | 3803 | C | LYS | A | 768 | 76.366 | 75.037 | 99.772 | 1.00 | 39.18 | 6 |
| ATOM | 3804 | O | LYS | A | 768 | 75.421 | 75.375 | 99.049 | 1.00 | 40.00 | 8 |
| ATOM | 3805 | N | GLN | A | 769 | 77.069 | 75.909 | 100.494 | 1.00 | 37.09 | 7 |
| ATOM | 3806 | CA | GLN | A | 769 | 76.735 | 77.330 | 100.486 | 1.00 | 37.20 | 6 |
| ATOM | 3807 | CB | GLN | A | 769 | 77.622 | 78.124 | 101.465 | 1.00 | 38.77 | 6 |
| ATOM | 3808 | CG | GLN | A | 769 | 77.606 | 77.648 | 102.913 | 1.00 | 41.12 | 6 |
| ATOM | 3809 | CD | GLN | A | 769 | 78.145 | 78.700 | 103.873 | 1.00 | 43.44 | 6 |
| ATOM | 3810 | OE1 | GLN | A | 769 | 79.125 | 79.379 | 103.583 | 1.00 | 45.95 | 8 |
| ATOM | 3811 | NE2 | GLN | A | 769 | 77.504 | 78.830 | 105.022 | 1.00 | 47.56 | 7 |
| ATOM | 3812 | C | GLN | A | 769 | 76.916 | 77.903 | 99.081 | 1.00 | 37.84 | 6 |
| ATOM | 3813 | O | GLN | A | 769 | 76.058 | 78.627 | 98.582 | 1.00 | 34.96 | 8 |
| ATOM | 3814 | N | LYS | A | 770 | 78.044 | 77.583 | 98.451 | 1.00 | 39.41 | 7 |
| ATOM | 3815 | CA | LYS | A | 770 | 78.323 | 78.079 | 97.108 | 1.00 | 41.73 | 6 |
| ATOM | 3816 | CB | LYS | A | 770 | 79.698 | 77.605 | 96.640 | 1.00 | 43.11 | 6 |
| ATOM | 3817 | CG | LYS | A | 770 | 80.860 | 78.220 | 97.394 | 1.00 | 42.84 | 6 |
| ATOM | 3818 | CD | LYS | A | 770 | 82.179 | 77.692 | 96.862 | 1.00 | 43.55 | 6 |
| ATOM | 3819 | CE | LYS | A | 770 | 83.361 | 78.302 | 97.590 | 1.00 | 44.60 | 6 |
| ATOM | 3820 | NZ | LYS | A | 770 | 83.419 | 79.776 | 97.397 | 1.00 | 44.90 | 7 |
| ATOM | 3821 | C | LYS | A | 770 | 77.257 | 77.632 | 96.110 | 1.00 | 42.27 | 6 |
| ATOM | 3822 | O | LYS | A | 770 | 76.839 | 78.412 | 95.253 | 1.00 | 41.85 | 8 |
| ATOM | 3823 | N | LEU | A | 771 | 76.818 | 76.380 | 96.229 | 1.00 | 42.21 | 7 |
| ATOM | 3824 | CA | LEU | A | 771 | 75.792 | 75.847 | 95.339 | 1.00 | 43.45 | 6 |
| ATOM | 3825 | CB | LEU | A | 771 | 75.640 | 74.335 | 95.537 | 1.00 | 40.02 | 6 |
| ATOM | 3826 | CG | LEU | A | 771 | 76.824 | 73.473 | 95.076 | 1.00 | 37.80 | 6 |
| ATOM | 3827 | CD1 | LEU | A | 771 | 76.514 | 71.997 | 95.290 | 1.00 | 35.60 | 6 |
| ATOM | 3828 | CD2 | LEU | A | 771 | 77.103 | 73.747 | 93.606 | 1.00 | 36.43 | 6 |
| ATOM | 3829 | C | LEU | A | 771 | 74.455 | 76.540 | 95.581 | 1.00 | 46.60 | 6 |
| ATOM | 3830 | O | LEU | A | 771 | 73.726 | 76.850 | 94.634 | 1.00 | 45.26 | 8 |
| ATOM | 3831 | N | GLU | A | 772 | 74.134 | 76.779 | 96.850 | 1.00 | 49.24 | 7 |
| ATOM | 3832 | CA | GLU | A | 772 | 72.891 | 77.450 | 97.210 | 1.00 | 52.60 | 6 |
| ATOM | 3833 | CB | GLU | A | 772 | 72.811 | 77.667 | 98.724 | 1.00 | 54.62 | 6 |
| ATOM | 3834 | CG | GLU | A | 772 | 72.402 | 76.445 | 99.530 | 1.00 | 58.60 | 6 |
| ATOM | 3835 | CD | GLU | A | 772 | 70.932 | 76.087 | 99.359 | 1.00 | 61.85 | 6 |
| ATOM | 3836 | OE1 | GLU | A | 772 | 70.077 | 76.979 | 99.554 | 1.00 | 63.98 | 8 |
| ATOM | 3837 | OE2 | GLU | A | 772 | 70.627 | 74.915 | 99.040 | 1.00 | 61.89 | 8 |
| ATOM | 3838 | C | GLU | A | 772 | 72.813 | 78.798 | 96.504 | 1.00 | 53.53 | 6 |
| ATOM | 3839 | O | GLU | A | 772 | 71.757 | 79.192 | 96.015 | 1.00 | 54.00 | 8 |
| ATOM | 3840 | N | ASN | A | 773 | 73.939 | 79.499 | 96.448 | 1.00 | 54.36 | 7 |
| ATOM | 3841 | CA | ASN | A | 773 | 73.983 | 80.797 | 95.800 | 1.00 | 57.04 | 6 |
| ATOM | 3842 | CB | ASN | A | 773 | 75.333 | 81.471 | 96.055 | 1.00 | 57.86 | 6 |
| ATOM | 3843 | CG | ASN | A | 773 | 75.531 | 81.842 | 97.514 | 1.00 | 59.86 | 6 |
| ATOM | 3844 | OD1 | ASN | A | 773 | 76.572 | 82.373 | 97.899 | 1.00 | 60.74 | 8 |
| ATOM | 3845 | ND2 | ASN | A | 773 | 74.524 | 81.562 | 98.337 | 1.00 | 60.42 | 7 |
| ATOM | 3846 | C | ASN | A | 773 | 73.716 | 80.681 | 94.302 | 1.00 | 58.60 | 6 |
| ATOM | 3847 | O | ASN | A | 773 | 72.982 | 81.492 | 93.736 | 1.00 | 58.99 | 8 |
| ATOM | 3848 | N | LEU | A | 774 | 74.304 | 79.672 | 93.661 | 1.00 | 59.50 | 7 |
| ATOM | 3849 | CA | LEU | A | 774 | 74.075 | 79.467 | 92.228 | 1.00 | 60.33 | 6 |
| ATOM | 3850 | CB | LEU | A | 774 | 74.965 | 78.307 | 91.680 | 1.00 | 59.59 | 6 |
| ATOM | 3851 | CG | LEU | A | 774 | 76.487 | 78.451 | 91.672 | 1.00 | 59.52 | 6 |
| ATOM | 3852 | CD1 | LEU | A | 774 | 77.131 | 77.286 | 90.946 | 1.00 | 59.18 | 6 |
| ATOM | 3853 | CD2 | LEU | A | 774 | 76.879 | 79.765 | 91.022 | 1.00 | 58.36 | 6 |
| ATOM | 3854 | C | LEU | A | 774 | 72.615 | 79.119 | 91.939 | 1.00 | 61.58 | 6 |
| ATOM | 3855 | O | LEU | A | 774 | 71.963 | 79.806 | 91.145 | 1.00 | 62.38 | 8 |
| ATOM | 3856 | N | GLN | A | 775 | 72.107 | 78.047 | 92.572 | 1.00 | 62.93 | 7 |
| ATOM | 3857 | CA | GLN | A | 775 | 70.725 | 77.516 | 92.437 | 1.00 | 64.63 | 6 |
| ATOM | 3858 | CB | GLN | A | 775 | 70.333 | 76.759 | 93.700 | 1.00 | 63.69 | 6 |
| ATOM | 3859 | CG | GLN | A | 775 | 69.060 | 75.960 | 93.534 | 1.00 | 63.39 | 6 |
| ATOM | 3860 | CD | GLN | A | 775 | 69.335 | 74.784 | 92.627 | 1.00 | 63.58 | 6 |
| ATOM | 3861 | OE1 | GLN | A | 775 | 69.786 | 74.942 | 91.496 | 1.00 | 63.72 | 8 |
| ATOM | 3862 | NE2 | GLN | A | 775 | 69.116 | 73.519 | 92.924 | 1.00 | 63.03 | 7 |
| ATOM | 3863 | C | GLN | A | 775 | 69.696 | 78.595 | 92.188 | 1.00 | 66.62 | 6 |
| ATOM | 3864 | O | GLN | A | 775 | 69.249 | 78.842 | 91.061 | 1.00 | 66.42 | 8 |
| ATOM | 3865 | N | ASN | A | 776 | 69.357 | 79.245 | 93.291 | 1.00 | 69.46 | 7 |
| ATOM | 3866 | CA | ASN | A | 776 | 68.421 | 80.352 | 93.293 | 1.00 | 72.25 | 6 |
| ATOM | 3867 | CB | ASN | A | 776 | 68.407 | 80.901 | 94.726 | 1.00 | 73.02 | 6 |
| ATOM | 3868 | CG | ASN | A | 776 | 67.896 | 79.797 | 95.666 | 1.00 | 74.84 | 6 |
| ATOM | 3869 | OD1 | ASN | A | 776 | 66.722 | 79.434 | 95.605 | 1.00 | 75.60 | 8 |
| ATOM | 3870 | ND2 | ASN | A | 776 | 68.764 | 79.283 | 96.514 | 1.00 | 75.30 | 7 |
| ATOM | 3871 | C | ASN | A | 776 | 68.706 | 81.302 | 92.059 | 1.00 | 73.56 | 6 |
| ATOM | 3872 | O | ASN | A | 776 | 69.831 | 81.277 | 91.570 | 1.00 | 74.49 | 8 |
| ATOM | 3873 | N | SER | A | 777 | 67.748 | 82.110 | 91.544 | 1.00 | 74.25 | 7 |
| ATOM | 3874 | CA | SER | A | 777 | 67.879 | 82.935 | 90.273 | 1.00 | 74.53 | 6 |
| ATOM | 3875 | CB | SER | A | 777 | 66.997 | 84.179 | 90.360 | 1.00 | 75.23 | 6 |
| ATOM | 3876 | OG | SER | A | 777 | 66.780 | 84.739 | 89.072 | 1.00 | 73.47 | 8 |
| ATOM | 3877 | C | SER | A | 777 | 69.256 | 83.384 | 89.763 | 1.00 | 75.03 | 6 |
| ATOM | 3878 | O | SER | A | 777 | 69.609 | 84.571 | 89.851 | 1.00 | 75.46 | 8 |
| ATOM | 3879 | N | GLN | A | 778 | 70.001 | 82.418 | 89.244 | 1.00 | 75.28 | 7 |
| ATOM | 3880 | CA | GLN | A | 778 | 71.310 | 82.593 | 88.586 | 1.00 | 75.03 | 6 |
| ATOM | 3881 | CB | GLN | A | 778 | 72.533 | 82.780 | 89.547 | 1.00 | 75.82 | 6 |
| ATOM | 3882 | CG | GLN | A | 778 | 73.552 | 83.851 | 89.057 | 1.00 | 77.52 | 6 |
| ATOM | 3883 | CD | GLN | A | 778 | 74.962 | 83.824 | 89.659 | 1.00 | 78.46 | 6 |
| ATOM | 3884 | OE1 | GLN | A | 778 | 75.891 | 83.253 | 89.087 | 1.00 | 78.53 | 8 |
| ATOM | 3885 | NE2 | GLN | A | 778 | 75.335 | 84.398 | 90.801 | 1.00 | 79.22 | 7 |
| ATOM | 3886 | C | GLN | A | 778 | 71.469 | 81.332 | 87.747 | 1.00 | 73.94 | 6 |
| ATOM | 3887 | O | GLN | A | 778 | 71.313 | 81.338 | 86.534 | 1.00 | 74.20 | 8 |
| ATOM | 3888 | N | LEU | A | 779 | 71.785 | 80.245 | 88.422 | 1.00 | 72.78 | 7 |
| ATOM | 3889 | CA | LEU | A | 779 | 71.982 | 78.947 | 87.756 | 1.00 | 71.39 | 6 |
| ATOM | 3890 | CB | LEU | A | 779 | 72.505 | 77.925 | 88.730 | 1.00 | 72.21 | 6 |
| ATOM | 3891 | CG | LEU | A | 779 | 72.665 | 76.517 | 88.169 | 1.00 | 72.85 | 6 |
| ATOM | 3892 | CD1 | LEU | A | 779 | 73.683 | 75.736 | 88.987 | 1.00 | 72.47 | 6 |
| ATOM | 3893 | CD2 | LEU | A | 779 | 71.314 | 75.791 | 88.140 | 1.00 | 72.76 | 6 |
| ATOM | 3894 | C | LEU | A | 779 | 70.687 | 78.471 | 87.110 | 1.00 | 69.63 | 6 |
| ATOM | 3895 | O | LEU | A | 779 | 69.593 | 78.657 | 87.615 | 1.00 | 70.11 | 8 |
| ATOM | 3896 | N | PRO | A | 780 | 70.890 | 77.853 | 85.942 | 1.00 | 67.91 | 7 |
| ATOM | 3897 | CD | PRO | A | 780 | 71.884 | 78.390 | 84.991 | 1.00 | 68.03 | 6 |
| ATOM | 3898 | CA | PRO | A | 780 | 69.727 | 77.430 | 85.114 | 1.00 | 65.90 | 6 |
| ATOM | 3899 | CB | PRO | A | 780 | 70.385 | 76.850 | 83.866 | 1.00 | 66.31 | 6 |
| ATOM | 3900 | CG | PRO | A | 780 | 71.532 | 77.770 | 83.669 | 1.00 | 67.63 | 6 |
| ATOM | 3901 | C | PRO | A | 780 | 68.672 | 76.543 | 85.679 | 1.00 | 63.40 | 6 |
| ATOM | 3902 | O | PRO | A | 780 | 68.844 | 75.861 | 86.677 | 1.00 | 64.20 | 8 |
| ATOM | 3903 | N | GLU | A | 781 | 67.571 | 76.592 | 84.992 | 1.00 | 60.69 | 7 |
| ATOM | 3904 | CA | GLU | A | 781 | 66.494 | 75.708 | 85.309 | 1.00 | 58.15 | 6 |
| ATOM | 3905 | CB | GLU | A | 781 | 65.159 | 76.214 | 84.792 | 1.00 | 59.12 | 6 |
| ATOM | 3906 | CG | GLU | A | 781 | 64.722 | 77.532 | 85.410 | 1.00 | 61.94 | 6 |
| ATOM | 3907 | CD | GLU | A | 781 | 63.224 | 77.756 | 85.363 | 1.00 | 64.17 | 6 |
| ATOM | 3908 | OE1 | GLU | A | 781 | 62.607 | 77.442 | 84.321 | 1.00 | 65.21 | 8 |
| ATOM | 3909 | OE2 | GLU | A | 781 | 62.666 | 78.252 | 86.364 | 1.00 | 64.28 | 8 |
| ATOM | 3910 | C | GLU | A | 781 | 66.884 | 74.383 | 84.683 | 1.00 | 54.42 | 6 |
| ATOM | 3911 | O | GLU | A | 781 | 66.543 | 73.345 | 85.241 | 1.00 | 54.46 | 8 |
| ATOM | 3912 | N | SER | A | 782 | 67.576 | 74.366 | 83.531 | 1.00 | 49.82 | 7 |
| ATOM | 3913 | CA | SER | A | 782 | 67.963 | 73.094 | 82.935 | 1.00 | 45.58 | 6 |
| ATOM | 3914 | CB | SER | A | 782 | 66.739 | 72.385 | 82.355 | 1.00 | 45.52 | 6 |
| ATOM | 3915 | OG | SER | A | 782 | 66.098 | 73.186 | 81.382 | 1.00 | 47.08 | 8 |
| ATOM | 3916 | C | SER | A | 782 | 69.036 | 73.250 | 81.859 | 1.00 | 41.51 | 6 |
| ATOM | 3917 | O | SER | A | 782 | 69.379 | 74.361 | 81.451 | 1.00 | 41.53 | 8 |
| ATOM | 3918 | N | PHE | A | 783 | 69.574 | 72.121 | 81.417 | 1.00 | 37.19 | 7 |
| ATOM | 3919 | CA | PHE | A | 783 | 70.614 | 72.111 | 80.401 | 1.00 | 33.67 | 6 |
| ATOM | 3920 | CB | PHE | A | 783 | 71.916 | 72.694 | 80.964 | 1.00 | 33.31 | 6 |
| ATOM | 3921 | CG | PHE | A | 783 | 72.504 | 71.897 | 82.106 | 1.00 | 34.50 | 6 |
| ATOM | 3922 | CD1 | PHE | A | 783 | 72.018 | 72.043 | 83.403 | 1.00 | 35.11 | 6 |
| ATOM | 3923 | CD2 | PHE | A | 783 | 73.539 | 70.993 | 81.880 | 1.00 | 34.92 | 6 |
| ATOM | 3924 | CE1 | PHE | A | 783 | 72.555 | 71.299 | 84.464 | 1.00 | 35.01 | 6 |
| ATOM | 3925 | CE2 | PHE | A | 783 | 74.083 | 70.240 | 82.935 | 1.00 | 35.93 | 6 |
| ATOM | 3926 | CZ | PHE | A | 783 | 73.589 | 70.395 | 84.225 | 1.00 | 34.85 | 6 |
| ATOM | 3927 | C | PHE | A | 783 | 70.846 | 70.671 | 79.971 | 1.00 | 33.09 | 6 |
| ATOM | 3928 | O | PHE | A | 783 | 70.482 | 69.736 | 80.692 | 1.00 | 32.31 | 8 |
| ATOM | 3929 | N | ARG | A | 784 | 71.435 | 70.482 | 78.792 | 1.00 | 31.67 | 7 |
| ATOM | 3930 | CA | ARG | A | 784 | 71.718 | 69.131 | 78.322 | 1.00 | 30.61 | 6 |
| ATOM | 3931 | CB | ARG | A | 784 | 71.867 | 69.076 | 76.804 | 1.00 | 32.75 | 6 |
| ATOM | 3932 | CG | ARG | A | 784 | 70.655 | 69.487 | 76.028 | 1.00 | 36.52 | 6 |
| ATOM | 3933 | CD | ARG | A | 784 | 70.864 | 69.197 | 74.546 | 1.00 | 39.76 | 6 |
| ATOM | 3934 | NE | ARG | A | 784 | 69.860 | 69.873 | 73.736 | 1.00 | 44.36 | 7 |
| ATOM | 3935 | CZ | ARG | A | 784 | 68.560 | 69.867 | 74.010 | 1.00 | 46.66 | 6 |
| ATOM | 3936 | NH1 | ARG | A | 784 | 68.106 | 69.215 | 75.074 | 1.00 | 46.34 | 7 |
| ATOM | 3937 | NH2 | ARG | A | 784 | 67.712 | 70.529 | 73.232 | 1.00 | 47.21 | 7 |
| ATOM | 3938 | C | ARG | A | 784 | 73.025 | 68.673 | 78.924 | 1.00 | 27.12 | 6 |
| ATOM | 3939 | O | ARG | A | 784 | 73.964 | 69.455 | 79.058 | 1.00 | 26.83 | 8 |
| ATOM | 3940 | N | VAL | A | 785 | 73.085 | 67.402 | 79.284 | 1.00 | 25.48 | 7 |
| ATOM | 3941 | CA | VAL | A | 785 | 74.302 | 66.848 | 79.845 | 1.00 | 24.76 | 6 |
| ATOM | 3942 | CB | VAL | A | 785 | 74.028 | 65.468 | 80.465 | 1.00 | 24.92 | 6 |
| ATOM | 3943 | CG1 | VAL | A | 785 | 75.315 | 64.890 | 81.048 | 1.00 | 24.86 | 6 |
| ATOM | 3944 | CG2 | VAL | A | 785 | 72.953 | 65.602 | 81.553 | 1.00 | 27.03 | 6 |
| ATOM | 3945 | C | VAL | A | 785 | 75.242 | 66.724 | 78.645 | 1.00 | 23.91 | 6 |
| ATOM | 3946 | O | VAL | A | 785 | 74.983 | 65.941 | 77.735 | 1.00 | 25.78 | 8 |
| ATOM | 3947 | N | PRO | A | 786 | 76.374 | 67.466 | 78.728 | 1.00 | 23.48 | 7 |
| ATOM | 3948 | CD | PRO | A | 786 | 76.368 | 68.800 | 79.313 | 1.00 | 22.61 | 6 |
| ATOM | 3949 | CA | PRO | A | 786 | 77.372 | 67.484 | 77.593 | 1.00 | 23.56 | 6 |
| ATOM | 3950 | CB | PRO | A | 786 | 78.486 | 68.359 | 78.126 | 1.00 | 23.40 | 6 |
| ATOM | 3951 | CG | PRO | A | 786 | 77.684 | 69.404 | 78.873 | 1.00 | 24.69 | 6 |
| ATOM | 3952 | C | PRO | A | 786 | 77.771 | 66.177 | 76.960 | 1.00 | 26.48 | 6 |
| ATOM | 3953 | O | PRO | A | 786 | 77.883 | 66.064 | 75.740 | 1.00 | 27.16 | 8 |
| ATOM | 3954 | N | TYR | A | 787 | 77.994 | 65.164 | 77.794 | 1.00 | 26.51 | 7 |
| ATOM | 3955 | CA | TYR | A | 787 | 78.411 | 63.853 | 77.319 | 1.00 | 27.39 | 6 |
| ATOM | 3956 | CB | TYR | A | 787 | 79.350 | 63.162 | 78.335 | 1.00 | 25.26 | 6 |
| ATOM | 3957 | CG | TYR | A | 787 | 78.699 | 63.034 | 79.675 | 1.00 | 28.23 | 6 |
| ATOM | 3958 | CD1 | TYR | A | 787 | 77.715 | 62.087 | 79.934 | 1.00 | 30.67 | 6 |
| ATOM | 3959 | CE1 | TYR | A | 787 | 77.115 | 62.001 | 81.183 | 1.00 | 32.43 | 6 |
| ATOM | 3960 | CD2 | TYR | A | 787 | 79.074 | 63.896 | 80.706 | 1.00 | 31.75 | 6 |
| ATOM | 3961 | CE2 | TYR | A | 787 | 78.484 | 63.817 | 81.956 | 1.00 | 32.50 | 6 |
| ATOM | 3962 | CZ | TYR | A | 787 | 77.509 | 62.872 | 82.190 | 1.00 | 32.90 | 6 |
| ATOM | 3963 | OH | TYR | A | 787 | 76.933 | 62.794 | 83.433 | 1.00 | 33.46 | 8 |
| ATOM | 3964 | C | TYR | A | 787 | 77.241 | 62.929 | 77.022 | 1.00 | 28.59 | 6 |
| ATOM | 3965 | O | TYR | A | 787 | 77.412 | 61.701 | 76.998 | 1.00 | 30.35 | 8 |
| ATOM | 3966 | N | ASP | A | 788 | 76.065 | 63.507 | 76.813 | 1.00 | 30.11 | 7 |
| ATOM | 3967 | CA | ASP | A | 788 | 74.856 | 62.734 | 76.476 | 1.00 | 29.60 | 6 |
| ATOM | 3968 | CB | ASP | A | 788 | 74.507 | 61.716 | 77.531 | 1.00 | 30.20 | 6 |
| ATOM | 3969 | CG | ASP | A | 788 | 73.394 | 60.866 | 76.988 | 1.00 | 32.48 | 6 |
| ATOM | 3970 | OD1 | ASP | A | 788 | 72.745 | 61.276 | 76.004 | 1.00 | 32.27 | 8 |
| ATOM | 3971 | OD2 | ASP | A | 788 | 73.174 | 59.774 | 77.541 | 1.00 | 32.51 | 8 |
| ATOM | 3972 | C | ASP | A | 788 | 73.701 | 63.676 | 76.257 | 1.00 | 28.89 | 6 |
| ATOM | 3973 | O | ASP | A | 788 | 72.934 | 64.038 | 77.153 | 1.00 | 30.12 | 8 |
| ATOM | 3974 | N | PRO | A | 789 | 73.652 | 64.049 | 74.998 | 1.00 | 27.45 | 7 |
| ATOM | 3975 | CD | PRO | A | 789 | 74.921 | 64.187 | 74.299 | 1.00 | 26.17 | 6 |
| ATOM | 3976 | CA | PRO | A | 789 | 72.733 | 65.078 | 74.514 | 1.00 | 26.76 | 6 |
| ATOM | 3977 | CB | PRO | A | 789 | 73.083 | 65.132 | 73.048 | 1.00 | 25.26 | 6 |
| ATOM | 3978 | CG | PRO | A | 789 | 74.573 | 64.950 | 73.068 | 1.00 | 24.36 | 6 |
| ATOM | 3979 | C | PRO | A | 789 | 71.275 | 64.941 | 74.895 | 1.00 | 27.24 | 6 |
| ATOM | 3980 | O | PRO | A | 789 | 70.593 | 65.905 | 75.247 | 1.00 | 29.34 | 8 |
| ATOM | 3981 | N | GLY | A | 790 | 70.826 | 63.691 | 74.801 | 1.00 | 28.50 | 7 |
| ATOM | 3982 | CA | GLY | A | 790 | 69.438 | 63.339 | 75.058 | 1.00 | 31.00 | 6 |
| ATOM | 3983 | C | GLY | A | 790 | 69.013 | 63.550 | 76.492 | 1.00 | 33.67 | 6 |
| ATOM | 3984 | O | GLY | A | 790 | 67.822 | 63.484 | 76.841 | 1.00 | 33.95 | 8 |
| ATOM | 3985 | N | LEU | A | 791 | 69.993 | 63.824 | 77.315 | 1.00 | 33.18 | 7 |
| ATOM | 3986 | CA | LEU | A | 791 | 69.770 | 63.993 | 78.744 | 1.00 | 33.86 | 6 |
| ATOM | 3987 | CB | LEU | A | 791 | 70.904 | 63.360 | 79.557 | 1.00 | 34.55 | 6 |
| ATOM | 3988 | CG | LEU | A | 791 | 70.732 | 61.926 | 80.043 | 1.00 | 38.86 | 6 |
| ATOM | 3989 | CD1 | LEU | A | 791 | 71.771 | 61.603 | 81.111 | 1.00 | 39.50 | 6 |
| ATOM | 3990 | CD2 | LEU | A | 791 | 69.337 | 61.699 | 80.594 | 1.00 | 38.23 | 6 |
| ATOM | 3991 | C | LEU | A | 791 | 69.620 | 65.455 | 79.141 | 1.00 | 32.73 | 6 |
| ATOM | 3992 | O | LEU | A | 791 | 70.469 | 66.311 | 78.864 | 1.00 | 29.98 | 8 |
| ATOM | 3993 | N | LYS | A | 792 | 68.496 | 65.732 | 79.808 | 1.00 | 34.39 | 7 |
| ATOM | 3994 | CA | LYS | A | 792 | 68.192 | 67.074 | 80.325 | 1.00 | 35.90 | 6 |
| ATOM | 3995 | CB | LYS | A | 792 | 66.824 | 67.566 | 79.882 | 1.00 | 39.38 | 6 |
| ATOM | 3996 | CG | LYS | A | 792 | 66.861 | 68.638 | 78.808 | 1.00 | 44.79 | 6 |
| ATOM | 3997 | CD | LYS | A | 792 | 67.434 | 69.951 | 79.316 | 1.00 | 48.94 | 6 |
| ATOM | 3998 | CE | LYS | A | 792 | 67.228 | 71.067 | 78.304 | 1.00 | 50.78 | 6 |
| ATOM | 3999 | NZ | LYS | A | 792 | 65.779 | 71.348 | 78.089 | 1.00 | 52.65 | 7 |
| ATOM | 4000 | C | LYS | A | 792 | 68.290 | 67.121 | 81.873 | 1.00 | 35.39 | 6 |
| ATOM | 4001 | O | LYS | A | 792 | 67.550 | 66.431 | 82.571 | 1.00 | 33.43 | 8 |
| ATOM | 4002 | N | ALA | A | 793 | 69.211 | 67.933 | 82.380 | 1.00 | 36.45 | 7 |
| ATOM | 4003 | CA | ALA | A | 793 | 69.391 | 68.075 | 83.820 | 1.00 | 37.53 | 6 |
| ATOM | 4004 | CB | ALA | A | 793 | 70.868 | 68.257 | 84.150 | 1.00 | 37.18 | 6 |
| ATOM | 4005 | C | ALA | A | 793 | 68.585 | 69.263 | 84.347 | 1.00 | 38.45 | 6 |
| ATOM | 4006 | O | ALA | A | 793 | 68.537 | 70.325 | 83.724 | 1.00 | 37.12 | 8 |
| ATOM | 4007 | N | GLY | A | 794 | 67.959 | 69.073 | 85.505 | 1.00 | 40.25 | 7 |
| ATOM | 4008 | CA | GLY | A | 794 | 67.171 | 70.129 | 86.105 | 1.00 | 40.58 | 6 |
| ATOM | 4009 | C | GLY | A | 794 | 67.934 | 70.792 | 87.228 | 1.00 | 41.08 | 6 |
| ATOM | 4010 | O | GLY | A | 794 | 69.138 | 71.044 | 87.111 | 1.00 | 41.92 | 8 |
| ATOM | 4011 | N | ALA | A | 795 | 67.240 | 71.061 | 88.328 | 1.00 | 41.21 | 7 |
| ATOM | 4012 | CA | ALA | A | 795 | 67.858 | 71.703 | 89.482 | 1.00 | 40.04 | 6 |
| ATOM | 4013 | CB | ALA | A | 795 | 66.780 | 72.234 | 90.425 | 1.00 | 40.19 | 6 |
| ATOM | 4014 | C | ALA | A | 795 | 68.789 | 70.767 | 90.239 | 1.00 | 38.81 | 6 |
| ATOM | 4015 | O | ALA | A | 795 | 68.648 | 69.545 | 90.189 | 1.00 | 37.12 | 8 |
| ATOM | 4016 | N | LEU | A | 796 | 69.748 | 71.366 | 90.935 | 1.00 | 37.96 | 7 |
| ATOM | 4017 | CA | LEU | A | 796 | 70.708 | 70.621 | 91.735 | 1.00 | 37.80 | 6 |
| ATOM | 4018 | CB | LEU | A | 796 | 71.844 | 71.541 | 92.189 | 1.00 | 38.03 | 6 |
| ATOM | 4019 | CG | LEU | A | 796 | 72.997 | 71.838 | 91.236 | 1.00 | 37.56 | 6 |
| ATOM | 4020 | CD1 | LEU | A | 796 | 73.612 | 73.181 | 91.574 | 1.00 | 36.92 | 6 |
| ATOM | 4021 | CD2 | LEU | A | 796 | 74.024 | 70.716 | 91.337 | 1.00 | 37.37 | 6 |
| ATOM | 4022 | C | LEU | A | 796 | 70.030 | 70.052 | 92.971 | 1.00 | 37.40 | 6 |
| ATOM | 4023 | O | LEU | A | 796 | 69.133 | 70.672 | 93.542 | 1.00 | 38.32 | 8 |
| ATOM | 4024 | N | ALA | A | 797 | 70.457 | 68.859 | 93.364 | 1.00 | 36.73 | 7 |
| ATOM | 4025 | CA | ALA | A | 797 | 69.953 | 68.200 | 94.562 | 1.00 | 37.48 | 6 |
| ATOM | 4026 | CB | ALA | A | 797 | 69.798 | 66.710 | 94.314 | 1.00 | 37.45 | 6 |
| ATOM | 4027 | C | ALA | A | 797 | 71.090 | 68.467 | 95.551 | 1.00 | 38.36 | 6 |
| ATOM | 4028 | O | ALA | A | 797 | 71.875 | 67.574 | 95.877 | 1.00 | 36.66 | 8 |
| ATOM | 4029 | N | ILE | A | 798 | 71.175 | 69.718 | 95.994 | 1.00 | 39.34 | 7 |
| ATOM | 4030 | CA | ILE | A | 798 | 72.238 | 70.174 | 96.884 | 1.00 | 40.04 | 6 |
| ATOM | 4031 | CB | ILE | A | 798 | 71.979 | 71.637 | 97.334 | 1.00 | 39.14 | 6 |
| ATOM | 4032 | CG2 | ILE | A | 798 | 73.177 | 72.175 | 98.128 | 1.00 | 39.45 | 6 |
| ATOM | 4033 | CG1 | ILE | A | 798 | 71.777 | 72.515 | 96.092 | 1.00 | 37.29 | 6 |
| ATOM | 4034 | CD1 | ILE | A | 798 | 71.679 | 73.999 | 96.375 | 1.00 | 34.88 | 6 |
| ATOM | 4035 | C | ILE | A | 798 | 72.523 | 69.296 | 98.094 | 1.00 | 41.31 | 6 |
| ATOM | 4036 | O | ILE | A | 798 | 73.678 | 69.174 | 98.511 | 1.00 | 41.00 | 8 |
| ATOM | 4037 | N | GLU | A | 799 | 71.485 | 68.672 | 98.641 | 1.00 | 42.02 | 7 |
| ATOM | 4038 | CA | GLU | A | 799 | 71.645 | 67.807 | 99.801 | 1.00 | 44.18 | 6 |
| ATOM | 4039 | CB | GLU | A | 799 | 70.282 | 67.382 | 100.360 | 1.00 | 46.93 | 6 |
| ATOM | 4040 | CG | GLU | A | 799 | 69.399 | 68.535 | 100.827 | 1.00 | 51.61 | 6 |
| ATOM | 4041 | CD | GLU | A | 799 | 68.595 | 69.164 | 99.697 | 1.00 | 53.73 | 6 |
| ATOM | 4042 | OE1 | GLU | A | 799 | 69.202 | 69.696 | 98.742 | 1.00 | 53.92 | 8 |
| ATOM | 4043 | OE2 | GLU | A | 799 | 67.346 | 69.125 | 99.769 | 1.00 | 55.13 | 8 |
| ATOM | 4044 | C | GLU | A | 799 | 72.443 | 66.568 | 99.447 | 1.00 | 43.69 | 6 |
| ATOM | 4045 | O | GLU | A | 799 | 73.007 | 65.920 | 100.321 | 1.00 | 45.36 | 8 |
| ATOM | 4046 | N | LYS | A | 800 | 72.459 | 66.111 | 98.192 | 1.00 | 42.80 | 7 |
| ATOM | 4047 | CA | LYS | A | 800 | 73.163 | 64.838 | 97.811 | 1.00 | 41.11 | 6 |
| ATOM | 4048 | CB | LYS | A | 800 | 72.372 | 64.094 | 96.743 | 1.00 | 43.81 | 6 |
| ATOM | 4049 | CG | LYS | A | 800 | 71.182 | 63.348 | 97.269 | 1.00 | 47.82 | 6 |
| ATOM | 4050 | CD | LYS | A | 800 | 70.634 | 62.423 | 96.204 | 1.00 | 52.02 | 6 |
| ATOM | 4051 | CE | LYS | A | 800 | 70.188 | 61.100 | 96.792 | 1.00 | 54.37 | 6 |
| ATOM | 4052 | NZ | LYS | A | 800 | 69.394 | 60.294 | 95.823 | 1.00 | 57.39 | 7 |
| ATOM | 4053 | C | LYS | A | 800 | 74.535 | 65.079 | 97.243 | 1.00 | 38.66 | 6 |
| ATOM | 4054 | O | LYS | A | 800 | 75.268 | 64.150 | 96.919 | 1.00 | 38.72 | 8 |
| ATOM | 4055 | N | CYS | A | 801 | 74.854 | 66.357 | 97.121 | 1.00 | 36.79 | 7 |
| ATOM | 4056 | CA | CYS | A | 801 | 76.123 | 66.707 | 96.569 | 1.00 | 35.93 | 6 |
| ATOM | 4057 | CB | CYS | A | 801 | 75.993 | 67.975 | 95.728 | 1.00 | 33.45 | 6 |
| ATOM | 4058 | SG | CYS | A | 801 | 74.821 | 67.842 | 94.342 | 1.00 | 34.86 | 16 |
| ATOM | 4059 | C | CYS | A | 801 | 77.179 | 66.815 | 97.670 | 1.00 | 36.63 | 6 |
| ATOM | 4060 | O | CYS | A | 801 | 76.858 | 66.924 | 98.853 | 1.00 | 39.22 | 8 |
| ATOM | 4061 | N | LYS | A | 802 | 78.471 | 66.755 | 97.253 | 1.00 | 35.63 | 7 |
| ATOM | 4062 | CA | LYS | A | 802 | 79.614 | 66.779 | 98.170 | 1.00 | 36.28 | 6 |
| ATOM | 4063 | CB | LYS | A | 802 | 79.586 | 65.543 | 99.090 | 1.00 | 36.89 | 6 |
| ATOM | 4064 | CG | LYS | A | 802 | 80.133 | 64.255 | 98.448 | 1.00 | 39.74 | 6 |
| ATOM | 4065 | CD | LYS | A | 802 | 79.405 | 62.998 | 98.909 | 1.00 | 44.19 | 6 |
| ATOM | 4066 | CE | LYS | A | 802 | 79.888 | 61.768 | 98.160 | 1.00 | 46.18 | 6 |
| ATOM | 4067 | NZ | LYS | A | 802 | 79.331 | 60.506 | 98.736 | 1.00 | 46.54 | 7 |
| ATOM | 4068 | C | LYS | A | 802 | 80.918 | 66.820 | 97.378 | 1.00 | 34.53 | 6 |
| ATOM | 4069 | O | LYS | A | 802 | 80.932 | 66.485 | 96.192 | 1.00 | 33.26 | 8 |
| ATOM | 4070 | N | VAL | A | 803 | 82.031 | 67.242 | 98.040 | 1.00 | 59.31 | 7 |
| ATOM | 4071 | CA | VAL | A | 803 | 83.372 | 67.314 | 97.438 | 1.00 | 59.52 | 6 |
| ATOM | 4072 | CB | VAL | A | 803 | 84.308 | 68.307 | 98.169 | 1.00 | 61.03 | 6 |
| ATOM | 4073 | CG1 | VAL | A | 803 | 85.747 | 68.133 | 97.696 | 1.00 | 59.47 | 6 |
| ATOM | 4074 | CG2 | VAL | A | 803 | 83.830 | 69.738 | 97.964 | 1.00 | 60.86 | 6 |
| ATOM | 4075 | C | VAL | A | 803 | 84.036 | 65.982 | 97.465 | 1.00 | 60.95 | 6 |
| ATOM | 4076 | O | VAL | A | 803 | 84.589 | 65.578 | 98.488 | 1.00 | 61.46 | 8 |
| ATOM | 4077 | N | MET | A | 804 | 83.969 | 65.279 | 96.356 | 1.00 | 102.79 | 7 |
| ATOM | 4078 | CA | MET | A | 804 | 84.606 | 64.017 | 96.404 | 1.00 | 104.12 | 6 |
| ATOM | 4079 | CB | MET | A | 804 | 85.170 | 63.599 | 95.034 | 1.00 | 67.22 | 6 |
| ATOM | 4080 | CG | MET | A | 804 | 84.097 | 63.207 | 94.020 | 1.00 | 68.55 | 6 |
| ATOM | 4081 | SD | MET | A | 804 | 84.313 | 61.513 | 93.426 | 1.00 | 68.12 | 16 |
| ATOM | 4082 | CE | MET | A | 804 | 85.720 | 61.725 | 92.339 | 1.00 | 71.38 | 6 |
| ATOM | 4083 | C | MET | A | 804 | 85.664 | 64.132 | 97.445 | 1.00 | 105.88 | 6 |
| ATOM | 4084 | O | MET | A | 804 | 86.039 | 65.214 | 97.879 | 1.00 | 105.81 | 8 |
| ATOM | 4085 | N | ALA | A | 805 | 86.130 | 62.989 | 97.837 | 1.00 | 114.21 | 7 |
| ATOM | 4086 | CA | ALA | A | 805 | 87.218 | 62.901 | 98.782 | 1.00 | 118.20 | 6 |
| ATOM | 4087 | CB | ALA | A | 805 | 86.803 | 62.152 | 100.042 | 1.00 | 88.92 | 6 |
| ATOM | 4088 | C | ALA | A | 805 | 88.337 | 62.205 | 98.098 | 1.00 | 119.89 | 6 |
| ATOM | 4089 | O | ALA | A | 805 | 88.345 | 60.971 | 97.973 | 1.00 | 120.97 | 8 |
| ATOM | 4090 | N | SER | A | 806 | 89.304 | 62.979 | 97.671 | 1.00 | 114.78 | 7 |
| ATOM | 4091 | CA | SER | A | 806 | 90.412 | 62.424 | 96.945 | 1.00 | 116.27 | 6 |
| ATOM | 4092 | CB | SER | A | 806 | 90.002 | 62.144 | 95.489 | 1.00 | 108.86 | 6 |
| ATOM | 4093 | OG | SER | A | 806 | 90.881 | 61.217 | 94.883 | 1.00 | 112.13 | 8 |
| ATOM | 4094 | C | SER | A | 806 | 91.580 | 63.395 | 96.971 | 1.00 | 115.79 | 6 |
| ATOM | 4095 | O | SER | A | 806 | 91.622 | 64.278 | 97.824 | 1.00 | 115.90 | 8 |
| ATOM | 4096 | N | LYS | A | 807 | 92.513 | 63.217 | 96.048 | 1.00 | 71.87 | 7 |
| ATOM | 4097 | CA | LYS | A | 807 | 93.728 | 64.037 | 95.982 | 1.00 | 72.71 | 6 |
| ATOM | 4098 | CB | LYS | A | 807 | 94.781 | 63.269 | 95.178 | 1.00 | 99.86 | 6 |
| ATOM | 4099 | CG | LYS | A | 807 | 95.066 | 61.866 | 95.711 | 1.00 | 102.72 | 6 |
| ATOM | 4100 | CD | LYS | A | 807 | 93.876 | 60.904 | 95.526 | 1.00 | 104.34 | 6 |
| ATOM | 4101 | CE | LYS | A | 807 | 94.047 | 59.539 | 96.203 | 1.00 | 104.39 | 6 |
| ATOM | 4102 | NZ | LYS | A | 807 | 92.847 | 58.671 | 96.023 | 1.00 | 106.17 | 7 |
| ATOM | 4103 | C | LYS | A | 807 | 93.468 | 65.456 | 95.469 | 1.00 | 71.07 | 6 |
| ATOM | 4104 | O | LYS | A | 807 | 94.037 | 66.440 | 95.962 | 1.00 | 70.57 | 8 |
| ATOM | 4105 | N | LYS | A | 808 | 92.594 | 65.565 | 94.467 | 1.00 | 107.91 | 7 |
| ATOM | 4106 | CA | LYS | A | 808 | 92.144 | 66.856 | 93.999 | 1.00 | 107.44 | 6 |
| ATOM | 4107 | CB | LYS | A | 808 | 92.372 | 67.103 | 92.522 | 1.00 | 138.65 | 6 |
| ATOM | 4108 | CG | LYS | A | 808 | 92.914 | 65.900 | 91.799 | 1.00 | 139.70 | 6 |
| ATOM | 4109 | CD | LYS | A | 808 | 93.037 | 66.170 | 90.307 | 1.00 | 139.32 | 6 |
| ATOM | 4110 | CE | LYS | A | 808 | 91.734 | 65.892 | 89.558 | 1.00 | 141.19 | 6 |
| ATOM | 4111 | NZ | LYS | A | 808 | 91.398 | 66.974 | 88.595 | 1.00 | 143.43 | 7 |
| ATOM | 4112 | C | LYS | A | 808 | 90.693 | 66.898 | 94.315 | 1.00 | 106.49 | 6 |
| ATOM | 4113 | O | LYS | A | 808 | 90.055 | 65.844 | 94.452 | 1.00 | 109.13 | 8 |
| ATOM | 4114 | N | LYS | A | 809 | 90.142 | 68.058 | 94.430 | 1.00 | 46.06 | 7 |
| ATOM | 4115 | CA | LYS | A | 809 | 88.840 | 67.977 | 94.944 | 1.00 | 41.76 | 6 |
| ATOM | 4116 | CB | LYS | A | 809 | 88.665 | 69.122 | 95.926 | 1.00 | 43.79 | 6 |
| ATOM | 4117 | CG | LYS | A | 809 | 89.635 | 69.048 | 97.122 | 1.00 | 45.62 | 6 |
| ATOM | 4118 | CD | LYS | A | 809 | 89.829 | 67.637 | 97.680 | 1.00 | 47.29 | 6 |
| ATOM | 4119 | CE | LYS | A | 809 | 90.771 | 67.621 | 98.886 | 1.00 | 50.46 | 6 |
| ATOM | 4120 | NZ | LYS | A | 809 | 90.626 | 66.382 | 99.697 | 1.00 | 52.09 | 7 |
| ATOM | 4121 | C | LYS | A | 809 | 87.704 | 67.801 | 93.968 | 1.00 | 37.60 | 6 |
| ATOM | 4122 | O | LYS | A | 809 | 87.214 | 68.739 | 93.326 | 1.00 | 36.33 | 8 |
| ATOM | 4123 | N | PRO | A | 810 | 87.298 | 66.548 | 93.896 | 1.00 | 34.24 | 7 |
| ATOM | 4124 | CD | PRO | A | 810 | 88.214 | 65.407 | 93.633 | 1.00 | 33.52 | 6 |
| ATOM | 4125 | CA | PRO | A | 810 | 86.108 | 66.312 | 93.134 | 1.00 | 32.27 | 6 |
| ATOM | 4126 | CB | PRO | A | 810 | 85.893 | 64.801 | 93.259 | 1.00 | 31.33 | 6 |
| ATOM | 4127 | CG | PRO | A | 810 | 87.299 | 64.293 | 93.194 | 1.00 | 32.35 | 6 |
| ATOM | 4128 | C | PRO | A | 810 | 84.991 | 67.195 | 93.560 | 1.00 | 29.93 | 6 |
| ATOM | 4129 | O | PRO | A | 810 | 85.064 | 67.974 | 94.505 | 1.00 | 27.91 | 8 |
| ATOM | 4130 | N | LEU | A | 811 | 83.965 | 67.054 | 92.822 | 1.00 | 27.11 | 7 |
| ATOM | 4131 | CA | LEU | A | 811 | 82.721 | 67.633 | 93.164 | 1.00 | 26.51 | 6 |
| ATOM | 4132 | CB | LEU | A | 811 | 82.398 | 68.953 | 92.429 | 1.00 | 26.29 | 6 |
| ATOM | 4133 | CG | LEU | A | 811 | 83.029 | 70.240 | 92.960 | 1.00 | 27.80 | 6 |
| ATOM | 4134 | CD1 | LEU | A | 811 | 82.023 | 71.364 | 92.947 | 1.00 | 24.33 | 6 |
| ATOM | 4135 | CD2 | LEU | A | 811 | 83.586 | 70.035 | 94.360 | 1.00 | 28.64 | 6 |
| ATOM | 4136 | C | LEU | A | 811 | 81.813 | 66.550 | 92.718 | 1.00 | 27.39 | 6 |
| ATOM | 4137 | O | LEU | A | 811 | 81.826 | 66.173 | 91.550 | 1.00 | 27.53 | 8 |
| ATOM | 4138 | N | TRP | A | 812 | 81.036 | 66.038 | 93.609 | 1.00 | 26.76 | 7 |
| ATOM | 4139 | CA | TRP | A | 812 | 80.081 | 65.028 | 93.251 | 1.00 | 28.47 | 6 |
| ATOM | 4140 | CB | TRP | A | 812 | 80.135 | 63.885 | 94.239 | 1.00 | 27.76 | 6 |
| ATOM | 4141 | CG | TRP | A | 812 | 79.083 | 62.831 | 94.199 | 1.00 | 28.02 | 6 |
| ATOM | 4142 | CD2 | TRP | A | 812 | 79.290 | 61.440 | 93.924 | 1.00 | 26.96 | 6 |
| ATOM | 4143 | CE2 | TRP | A | 812 | 78.040 | 60.801 | 94.041 | 1.00 | 27.44 | 6 |
| ATOM | 4144 | CE3 | TRP | A | 812 | 80.418 | 60.671 | 93.590 | 1.00 | 30.52 | 6 |
| ATOM | 4145 | CD1 | TRP | A | 812 | 77.750 | 62.977 | 94.453 | 1.00 | 27.21 | 6 |
| ATOM | 4146 | NE1 | TRP | A | 812 | 77.117 | 61.760 | 94.367 | 1.00 | 29.41 | 7 |
| ATOM | 4147 | CZ2 | TRP | A | 812 | 77.877 | 59.420 | 93.837 | 1.00 | 29.36 | 6 |
| ATOM | 4148 | CZ3 | TRP | A | 812 | 80.257 | 59.295 | 93.387 | 1.00 | 29.09 | 6 |
| ATOM | 4149 | CH2 | TRP | A | 812 | 78.994 | 58.687 | 93.512 | 1.00 | 28.23 | 6 |
| ATOM | 4150 | C | TRP | A | 812 | 78.741 | 65.741 | 93.268 | 1.00 | 31.95 | 6 |
| ATOM | 4151 | O | TRP | A | 812 | 78.151 | 65.968 | 94.338 | 1.00 | 33.57 | 8 |
| ATOM | 4152 | N | LEU | A | 813 | 78.255 | 66.088 | 92.077 | 1.00 | 29.34 | 7 |
| ATOM | 4153 | CA | LEU | A | 813 | 77.017 | 66.836 | 91.933 | 1.00 | 29.69 | 6 |
| ATOM | 4154 | CB | LEU | A | 813 | 77.215 | 68.005 | 90.961 | 1.00 | 27.65 | 6 |
| ATOM | 4155 | CG | LEU | A | 813 | 78.391 | 68.943 | 91.241 | 1.00 | 29.50 | 6 |
| ATOM | 4156 | CD1 | LEU | A | 813 | 78.437 | 70.025 | 90.177 | 1.00 | 25.59 | 6 |
| ATOM | 4157 | CD2 | LEU | A | 813 | 78.255 | 69.553 | 92.627 | 1.00 | 25.28 | 6 |
| ATOM | 4158 | C | LEU | A | 813 | 75.880 | 65.979 | 91.429 | 1.00 | 30.08 | 6 |
| ATOM | 4159 | O | LEU | A | 813 | 76.073 | 65.088 | 90.602 | 1.00 | 28.54 | 8 |
| ATOM | 4160 | N | GLU | A | 814 | 74.690 | 66.242 | 91.945 | 1.00 | 30.50 | 7 |
| ATOM | 4161 | CA | GLU | A | 814 | 73.521 | 65.500 | 91.514 | 1.00 | 31.58 | 6 |
| ATOM | 4162 | CB | GLU | A | 814 | 73.052 | 64.556 | 92.616 | 1.00 | 33.32 | 6 |
| ATOM | 4163 | CG | GLU | A | 814 | 73.879 | 63.279 | 92.685 | 1.00 | 37.75 | 6 |
| ATOM | 4164 | CD | GLU | A | 814 | 73.422 | 62.334 | 93.780 | 1.00 | 40.58 | 6 |
| ATOM | 4165 | OE1 | GLU | A | 814 | 72.199 | 62.224 | 94.003 | 1.00 | 43.36 | 8 |
| ATOM | 4166 | OE2 | GLU | A | 814 | 74.284 | 61.688 | 94.407 | 1.00 | 41.51 | 8 |
| ATOM | 4167 | C | GLU | A | 814 | 72.414 | 66.454 | 91.117 | 1.00 | 31.76 | 6 |
| ATOM | 4168 | O | GLU | A | 814 | 72.136 | 67.430 | 91.820 | 1.00 | 29.86 | 8 |
| ATOM | 4169 | N | PHE | A | 815 | 71.801 | 66.180 | 89.969 | 1.00 | 32.36 | 7 |
| ATOM | 4170 | CA | PHE | A | 815 | 70.715 | 67.007 | 89.461 | 1.00 | 31.93 | 6 |
| ATOM | 4171 | CB | PHE | A | 815 | 71.072 | 67.637 | 88.101 | 1.00 | 31.03 | 6 |
| ATOM | 4172 | CG | PHE | A | 815 | 72.403 | 68.341 | 88.068 | 1.00 | 31.37 | 6 |
| ATOM | 4173 | CD1 | PHE | A | 815 | 73.592 | 67.616 | 88.013 | 1.00 | 30.17 | 6 |
| ATOM | 4174 | CD2 | PHE | A | 815 | 72.466 | 69.732 | 88.064 | 1.00 | 29.19 | 6 |
| ATOM | 4175 | CE1 | PHE | A | 815 | 74.827 | 68.266 | 87.951 | 1.00 | 27.77 | 6 |
| ATOM | 4176 | CE2 | PHE | A | 815 | 73.695 | 70.387 | 88.002 | 1.00 | 29.22 | 6 |
| ATOM | 4177 | CZ | PHE | A | 815 | 74.878 | 69.647 | 87.944 | 1.00 | 27.08 | 6 |
| ATOM | 4178 | C | PHE | A | 815 | 69.478 | 66.141 | 89.268 | 1.00 | 33.40 | 6 |
| ATOM | 4179 | O | PHE | A | 815 | 69.574 | 64.930 | 89.043 | 1.00 | 33.14 | 8 |
| ATOM | 4180 | N | LYS | A | 816 | 68.311 | 66.761 | 89.368 | 1.00 | 36.21 | 7 |
| ATOM | 4181 | CA | LYS | A | 816 | 67.064 | 66.044 | 89.142 | 1.00 | 38.94 | 6 |
| ATOM | 4182 | CB | LYS | A | 816 | 65.889 | 66.791 | 89.784 | 1.00 | 42.45 | 6 |
| ATOM | 4183 | CG | LYS | A | 816 | 66.055 | 67.122 | 91.268 | 1.00 | 47.51 | 6 |
| ATOM | 4184 | CD | LYS | A | 816 | 64.747 | 67.664 | 91.843 | 1.00 | 50.66 | 6 |
| ATOM | 4185 | CE | LYS | A | 816 | 64.893 | 68.133 | 93.283 | 1.00 | 50.91 | 6 |
| ATOM | 4186 | NZ | LYS | A | 816 | 65.793 | 69.320 | 93.391 | 1.00 | 53.90 | 7 |
| ATOM | 4187 | C | LYS | A | 816 | 66.910 | 66.058 | 87.617 | 1.00 | 38.78 | 6 |
| ATOM | 4188 | O | LYS | A | 816 | 67.462 | 66.938 | 86.954 | 1.00 | 37.41 | 8 |
| ATOM | 4189 | N | CYS | A | 817 | 66.183 | 65.093 | 87.058 | 1.00 | 40.01 | 7 |
| ATOM | 4190 | CA | CYS | A | 817 | 65.982 | 65.055 | 85.606 | 1.00 | 41.19 | 6 |
| ATOM | 4191 | CB | CYS | A | 817 | 65.569 | 63.649 | 85.141 | 1.00 | 41.55 | 6 |
| ATOM | 4192 | SG | CYS | A | 817 | 65.320 | 63.481 | 83.331 | 1.00 | 48.24 | 16 |
| ATOM | 4193 | C | CYS | A | 817 | 64.891 | 66.061 | 85.259 | 1.00 | 40.39 | 6 |
| ATOM | 4194 | O | CYS | A | 817 | 63.835 | 66.071 | 85.875 | 1.00 | 41.00 | 8 |
| ATOM | 4195 | N | ALA | A | 818 | 65.147 | 66.904 | 84.269 | 1.00 | 41.12 | 7 |
| ATOM | 4196 | CA | ALA | A | 818 | 64.181 | 67.920 | 83.872 | 1.00 | 42.28 | 6 |
| ATOM | 4197 | CB | ALA | A | 818 | 64.899 | 69.075 | 83.179 | 1.00 | 40.20 | 6 |
| ATOM | 4198 | C | ALA | A | 818 | 63.055 | 67.405 | 82.978 | 1.00 | 43.10 | 6 |
| ATOM | 4199 | O | ALA | A | 818 | 62.313 | 68.201 | 82.405 | 1.00 | 44.37 | 8 |
| ATOM | 4200 | N | ASP | A | 819 | 62.890 | 66.087 | 82.855 | 1.00 | 44.35 | 7 |
| ATOM | 4201 | CA | ASP | A | 819 | 61.833 | 65.558 | 82.011 | 1.00 | 46.07 | 6 |
| ATOM | 4202 | CB | ASP | A | 819 | 62.430 | 64.671 | 80.912 | 1.00 | 47.24 | 6 |
| ATOM | 4203 | CG | ASP | A | 819 | 61.463 | 64.338 | 79.795 | 1.00 | 50.39 | 6 |
| ATOM | 4204 | OD1 | ASP | A | 819 | 60.275 | 64.120 | 80.097 | 1.00 | 50.62 | 8 |
| ATOM | 4205 | OD2 | ASP | A | 819 | 61.901 | 64.309 | 78.614 | 1.00 | 50.13 | 8 |
| ATOM | 4206 | C | ASP | A | 819 | 60.760 | 64.771 | 82.816 | 1.00 | 46.48 | 6 |
| ATOM | 4207 | O | ASP | A | 819 | 61.070 | 63.812 | 83.515 | 1.00 | 46.17 | 8 |
| ATOM | 4208 | N | PRO | A | 820 | 59.499 | 65.221 | 82.708 | 1.00 | 48.60 | 7 |
| ATOM | 4209 | CD | PRO | A | 820 | 59.244 | 66.633 | 82.913 | 1.00 | 49.48 | 6 |
| ATOM | 4210 | CA | PRO | A | 820 | 58.364 | 64.516 | 83.398 | 1.00 | 49.59 | 6 |
| ATOM | 4211 | CB | PRO | A | 820 | 57.159 | 65.341 | 83.062 | 1.00 | 48.89 | 6 |
| ATOM | 4212 | CG | PRO | A | 820 | 57.749 | 66.705 | 83.135 | 1.00 | 49.30 | 6 |
| ATOM | 4213 | C | PRO | A | 820 | 58.229 | 63.087 | 83.063 | 1.00 | 50.35 | 6 |
| ATOM | 4214 | O | PRO | A | 820 | 58.113 | 62.181 | 83.897 | 1.00 | 50.89 | 8 |
| ATOM | 4215 | N | THR | A | 821 | 58.247 | 62.922 | 81.751 | 1.00 | 50.45 | 7 |
| ATOM | 4216 | CA | THR | A | 821 | 58.076 | 61.671 | 81.125 | 1.00 | 50.10 | 6 |
| ATOM | 4217 | CB | THR | A | 821 | 58.032 | 61.891 | 79.617 | 1.00 | 51.30 | 6 |
| ATOM | 4218 | OG1 | THR | A | 821 | 59.348 | 62.250 | 79.158 | 1.00 | 48.74 | 8 |
| ATOM | 4219 | CG2 | THR | A | 821 | 57.046 | 62.998 | 79.273 | 1.00 | 50.18 | 6 |
| ATOM | 4220 | C | THR | A | 821 | 59.118 | 60.646 | 81.499 | 1.00 | 50.66 | 6 |
| ATOM | 4221 | O | THR | A | 821 | 58.993 | 59.461 | 81.160 | 1.00 | 49.58 | 8 |
| ATOM | 4222 | N | ALA | A | 822 | 60.153 | 61.085 | 82.218 | 1.00 | 51.42 | 7 |
| ATOM | 4223 | CA | ALA | A | 822 | 61.191 | 60.176 | 62.657 | 1.00 | 53.36 | 6 |
| ATOM | 4224 | CB | ALA | A | 822 | 62.120 | 60.841 | 63.666 | 1.00 | 53.59 | 6 |
| ATOM | 4225 | C | ALA | A | 822 | 60.552 | 58.924 | 83.270 | 1.00 | 54.99 | 6 |
| ATOM | 4226 | O | ALA | A | 822 | 59.474 | 58.984 | 83.882 | 1.00 | 55.32 | 8 |
| ATOM | 4227 | N | LEU | A | 823 | 61.228 | 57.811 | 83.086 | 1.00 | 57.90 | 7 |
| ATOM | 4228 | CA | LEU | A | 823 | 60.721 | 56.572 | 83.585 | 1.00 | 61.04 | 6 |
| ATOM | 4229 | CB | LEU | A | 823 | 61.221 | 55.472 | 82.687 | 1.00 | 62.72 | 6 |
| ATOM | 4230 | CG | LEU | A | 823 | 60.366 | 54.227 | 82.728 | 1.00 | 64.75 | 6 |
| ATOM | 4231 | CD1 | LEU | A | 823 | 58.946 | 54.544 | 82.289 | 1.00 | 66.14 | 6 |
| ATOM | 4232 | CD2 | LEU | A | 823 | 60.959 | 53.129 | 81.847 | 1.00 | 65.73 | 6 |
| ATOM | 4233 | C | LEU | A | 823 | 61.121 | 56.287 | 85.041 | 1.00 | 62.70 | 6 |
| ATOM | 4234 | O | LEU | A | 823 | 60.826 | 55.202 | 85.555 | 1.00 | 63.01 | 8 |
| ATOM | 4235 | N | SER | A | 824 | 61.817 | 57.236 | 85.718 | 1.00 | 63.27 | 7 |
| ATOM | 4236 | CA | SER | A | 824 | 62.339 | 57.082 | 87.102 | 1.00 | 63.57 | 6 |
| ATOM | 4237 | CB | SER | A | 824 | 63.710 | 56.395 | 87.082 | 1.00 | 63.48 | 6 |
| ATOM | 4238 | OG | SER | A | 824 | 64.219 | 56.222 | 88.385 | 1.00 | 63.75 | 8 |
| ATOM | 4239 | C | SER | A | 824 | 62.454 | 58.397 | 87.901 | 1.00 | 63.74 | 6 |
| ATOM | 4240 | O | SER | A | 824 | 61.989 | 59.447 | 87.458 | 1.00 | 63.75 | 8 |
| ATOM | 4241 | N | ASN | A | 825 | 63.077 | 58.317 | 89.070 | 1.00 | 64.17 | 7 |
| ATOM | 4242 | CA | ASN | A | 825 | 63.274 | 59.475 | 89.924 | 1.00 | 63.55 | 6 |
| ATOM | 4243 | CB | ASN | A | 825 | 62.623 | 59.224 | 91.285 | 1.00 | 65.66 | 6 |
| ATOM | 4244 | CG | ASN | A | 825 | 61.150 | 58.885 | 91.169 | 1.00 | 67.25 | 6 |
| ATOM | 4245 | OD1 | ASN | A | 825 | 60.318 | 59.756 | 90.907 | 1.00 | 69.18 | 8 |
| ATOM | 4246 | ND2 | ASN | A | 825 | 60.821 | 57.610 | 91.348 | 1.00 | 67.62 | 7 |
| ATOM | 4247 | C | ASN | A | 825 | 64.774 | 59.676 | 90.080 | 1.00 | 62.15 | 6 |
| ATOM | 4248 | O | ASN | A | 825 | 65.225 | 60.616 | 90.740 | 1.00 | 62.20 | 8 |
| ATOM | 4249 | N | GLU | A | 826 | 65.539 | 58.779 | 89.459 | 1.00 | 59.99 | 7 |
| ATOM | 4250 | CA | GLU | A | 826 | 66.995 | 58.833 | 89.497 | 1.00 | 57.32 | 6 |
| ATOM | 4251 | CB | GLU | A | 826 | 67.596 | 57.854 | 88.490 | 1.00 | 58.57 | 6 |
| ATOM | 4252 | CG | GLU | A | 826 | 67.419 | 56.394 | 88.819 | 1.00 | 62.13 | 6 |
| ATOM | 4253 | CD | GLU | A | 826 | 68.044 | 55.493 | 87.765 | 1.00 | 64.47 | 6 |
| ATOM | 4254 | OE1 | GLU | A | 826 | 68.054 | 54.258 | 87.965 | 1.00 | 65.40 | 8 |
| ATOM | 4255 | OE2 | GLU | A | 826 | 68.521 | 56.021 | 86.734 | 1.00 | 65.47 | 8 |
| ATOM | 4256 | C | GLU | A | 826 | 67.511 | 60.227 | 89.163 | 1.00 | 54.68 | 6 |
| ATOM | 4257 | O | GLU | A | 826 | 67.023 | 60.876 | 88.237 | 1.00 | 53.65 | 8 |
| ATOM | 4258 | N | THR | A | 827 | 68.499 | 60.686 | 89.922 | 1.00 | 51.47 | 7 |
| ATOM | 4259 | CA | THR | A | 827 | 69.082 | 61.989 | 89.660 | 1.00 | 48.17 | 6 |
| ATOM | 4260 | CB | THR | A | 827 | 69.636 | 62.659 | 90.938 | 1.00 | 49.20 | 6 |
| ATOM | 4261 | OG1 | THR | A | 827 | 70.558 | 61.771 | 91.581 | 1.00 | 50.25 | 8 |
| ATOM | 4262 | CG2 | THR | A | 827 | 68.506 | 63.018 | 91.892 | 1.00 | 50.74 | 6 |
| ATOM | 4263 | C | THR | A | 827 | 70.240 | 61.770 | 88.702 | 1.00 | 44.57 | 6 |
| ATOM | 4264 | O | THR | A | 827 | 70.752 | 60.659 | 88.570 | 1.00 | 43.13 | 8 |
| ATOM | 4265 | N | ILE | A | 828 | 70.642 | 62.833 | 88.025 | 1.00 | 39.70 | 7 |
| ATOM | 4266 | CA | ILE | A | 828 | 71.751 | 62.749 | 87.099 | 1.00 | 35.22 | 6 |
| ATOM | 4267 | CB | ILE | A | 828 | 71.555 | 63.725 | 85.936 | 1.00 | 34.44 | 6 |
| ATOM | 4268 | CG2 | ILE | A | 828 | 72.747 | 63.653 | 84.983 | 1.00 | 32.18 | 6 |
| ATOM | 4269 | CG1 | ILE | A | 828 | 70.239 | 63.386 | 85.221 | 1.00 | 33.81 | 6 |
| ATOM | 4270 | CD1 | ILE | A | 828 | 69.774 | 64.435 | 84.250 | 1.00 | 33.61 | 6 |
| ATOM | 4271 | C | ILE | A | 828 | 73.004 | 63.110 | 87.880 | 1.00 | 32.07 | 6 |
| ATOM | 4272 | O | ILE | A | 828 | 73.101 | 64.199 | 88.440 | 1.00 | 30.62 | 8 |
| ATOM | 4273 | N | GLY | A | 829 | 73.950 | 62.181 | 87.927 | 1.00 | 30.08 | 7 |
| ATOM | 4274 | CA | GLY | A | 829 | 75.180 | 62.416 | 88.650 | 1.00 | 30.32 | 6 |
| ATOM | 4275 | C | GLY | A | 829 | 76.315 | 62.838 | 87.747 | 1.00 | 30.04 | 6 |
| ATOM | 4276 | O | GLY | A | 829 | 76.572 | 62.219 | 86.714 | 1.00 | 30.92 | 8 |
| ATOM | 4277 | N | ILE | A | 830 | 76.994 | 63.906 | 88.141 | 1.00 | 28.55 | 7 |
| ATOM | 4278 | CA | ILE | A | 830 | 78.115 | 64.425 | 87.386 | 1.00 | 28.08 | 6 |
| ATOM | 4279 | CB | ILE | A | 830 | 77.747 | 65.685 | 86.597 | 1.00 | 28.19 | 6 |
| ATOM | 4280 | CG2 | ILE | A | 830 | 78.972 | 66.182 | 85.851 | 1.00 | 28.86 | 6 |
| ATOM | 4281 | CG1 | ILE | A | 830 | 76.578 | 65.403 | 85.650 | 1.00 | 27.05 | 6 |
| ATOM | 4282 | CD1 | ILE | A | 830 | 76.118 | 66.638 | 84.884 | 1.00 | 27.96 | 6 |
| ATOM | 4283 | C | ILE | A | 830 | 79.230 | 64.806 | 88.338 | 1.00 | 28.63 | 6 |
| ATOM | 4284 | O | ILE | A | 830 | 78.993 | 65.467 | 89.356 | 1.00 | 26.85 | 8 |
| ATOM | 4285 | N | ILE | A | 831 | 80.441 | 64.378 | 87.999 | 1.00 | 26.21 | 7 |
| ATOM | 4286 | CA | ILE | A | 831 | 81.615 | 64.679 | 88.785 | 1.00 | 26.38 | 6 |
| ATOM | 4287 | CB | ILE | A | 831 | 82.558 | 63.457 | 88.913 | 1.00 | 26.98 | 6 |
| ATOM | 4288 | CG2 | ILE | A | 831 | 83.956 | 63.889 | 89.361 | 1.00 | 26.06 | 6 |
| ATOM | 4289 | CG1 | ILE | A | 831 | 81.976 | 62.421 | 89.877 | 1.00 | 27.70 | 6 |
| ATOM | 4290 | CD1 | ILE | A | 831 | 82.790 | 61.153 | 89.977 | 1.00 | 26.98 | 6 |
| ATOM | 4291 | C | ILE | A | 831 | 82.402 | 65.769 | 88.116 | 1.00 | 27.70 | 6 |
| ATOM | 4292 | O | ILE | A | 831 | 82.733 | 65.669 | 86.936 | 1.00 | 28.99 | 8 |
| ATOM | 4293 | N | PHE | A | 832 | 82.693 | 66.816 | 88.866 | 1.00 | 27.17 | 7 |
| ATOM | 4294 | CA | PHE | A | 832 | 83.471 | 67.925 | 88.321 | 1.00 | 26.96 | 6 |
| ATOM | 4295 | CB | PHE | A | 832 | 82.826 | 69.275 | 88.587 | 1.00 | 30.60 | 6 |
| ATOM | 4296 | CG | PHE | A | 832 | 82.323 | 69.917 | 87.293 | 1.00 | 32.02 | 6 |
| ATOM | 4297 | CD1 | PHE | A | 832 | 81.036 | 69.643 | 86.854 | 1.00 | 35.35 | 6 |
| ATOM | 4298 | CD2 | PHE | A | 832 | 83.132 | 70.754 | 86.535 | 1.00 | 35.30 | 6 |
| ATOM | 4299 | CE1 | PHE | A | 832 | 80.556 | 70.195 | 85.670 | 1.00 | 38.23 | 6 |
| ATOM | 4300 | CE2 | PHE | A | 832 | 82.666 | 71.313 | 85.346 | 1.00 | 37.43 | 6 |
| ATOM | 4301 | CZ | PHE | A | 832 | 81.378 | 71.033 | 84.915 | 1.00 | 37.52 | 6 |
| ATOM | 4302 | C | PHE | A | 832 | 84.894 | 67.835 | 88.838 | 1.00 | 29.18 | 6 |
| ATOM | 4303 | O | PHE | A | 832 | 85.147 | 67.866 | 90.042 | 1.00 | 27.37 | 8 |
| ATOM | 4304 | N | LYS | A | 833 | 85.835 | 67.728 | 87.907 | 1.00 | 28.65 | 7 |
| ATOM | 4305 | CA | LYS | A | 833 | 87.203 | 67.520 | 88.326 | 1.00 | 30.32 | 6 |
| ATOM | 4306 | CB | LYS | A | 833 | 87.519 | 66.037 | 88.105 | 1.00 | 29.20 | 6 |
| ATOM | 4307 | CG | LYS | A | 833 | 89.000 | 65.711 | 88.174 | 1.00 | 29.35 | 6 |
| ATOM | 4308 | CD | LYS | A | 833 | 89.244 | 64.206 | 88.286 | 1.00 | 32.24 | 6 |
| ATOM | 4309 | CE | LYS | A | 833 | 88.859 | 63.495 | 87.004 | 1.00 | 35.61 | 6 |
| ATOM | 4310 | NZ | LYS | A | 833 | 88.954 | 62.010 | 87.135 | 1.00 | 39.32 | 7 |
| ATOM | 4311 | C | LYS | A | 833 | 88.299 | 68.408 | 87.706 | 1.00 | 30.09 | 6 |
| ATOM | 4312 | O | LYS | A | 833 | 88.294 | 68.753 | 86.523 | 1.00 | 27.79 | 8 |
| ATOM | 4313 | N | HIS | A | 834 | 89.243 | 68.739 | 88.617 | 1.00 | 29.77 | 7 |
| ATOM | 4314 | CA | HIS | A | 834 | 90.469 | 69.537 | 88.384 | 1.00 | 29.14 | 6 |
| ATOM | 4315 | CB | HIS | A | 834 | 90.437 | 70.915 | 89.075 | 1.00 | 31.68 | 6 |
| ATOM | 4316 | CG | HIS | A | 834 | 91.815 | 71.616 | 88.954 | 1.00 | 35.07 | 6 |
| ATOM | 4317 | CD2 | HIS | A | 834 | 92.932 | 71.588 | 89.753 | 1.00 | 36.22 | 6 |
| ATOM | 4318 | ND1 | HIS | A | 834 | 92.126 | 72.422 | 87.889 | 1.00 | 38.17 | 7 |
| ATOM | 4319 | CE1 | HIS | A | 834 | 93.376 | 72.869 | 88.016 | 1.00 | 38.63 | 6 |
| ATOM | 4320 | NE2 | HIS | A | 834 | 93.888 | 72.364 | 89.138 | 1.00 | 37.08 | 7 |
| ATOM | 4321 | C | HIS | A | 834 | 91.680 | 68.767 | 88.920 | 1.00 | 30.26 | 6 |
| ATOM | 4322 | O | HIS | A | 834 | 91.635 | 68.215 | 90.017 | 1.00 | 30.74 | 8 |
| ATOM | 4323 | N | GLY | A | 835 | 92.749 | 68.734 | 88.160 | 1.00 | 30.76 | 7 |
| ATOM | 4324 | CA | GLY | A | 835 | 93.941 | 68.032 | 88.579 | 1.00 | 32.62 | 6 |
| ATOM | 4325 | C | GLY | A | 835 | 94.338 | 66.964 | 87.576 | 1.00 | 33.45 | 6 |
| ATOM | 4326 | O | GLY | A | 835 | 95.495 | 66.549 | 87.501 | 1.00 | 34.55 | 8 |
| ATOM | 4327 | N | ASP | A | 836 | 93.378 | 66.527 | 86.799 | 1.00 | 33.08 | 7 |
| ATOM | 4328 | CA | ASP | A | 836 | 93.579 | 65.500 | 85.784 | 1.00 | 31.14 | 6 |
| ATOM | 4329 | CB | ASP | A | 836 | 92.609 | 64.349 | 85.961 | 1.00 | 33.88 | 6 |
| ATOM | 4330 | CG | ASP | A | 836 | 93.048 | 63.327 | 86.977 | 1.00 | 37.89 | 6 |
| ATOM | 4331 | OD1 | ASP | A | 836 | 93.706 | 63.710 | 87.955 | 1.00 | 38.81 | 8 |
| ATOM | 4332 | OD2 | ASP | A | 836 | 92.742 | 62.128 | 86.793 | 1.00 | 38.90 | 8 |
| ATOM | 4333 | C | ASP | A | 836 | 93.291 | 66.085 | 84.447 | 1.00 | 28.79 | 6 |
| ATOM | 4334 | O | ASP | A | 836 | 92.237 | 66.670 | 84.237 | 1.00 | 27.78 | 8 |
| ATOM | 4335 | N | ASP | A | 837 | 94.197 | 65.956 | 83.524 | 1.00 | 29.31 | 7 |
| ATOM | 4336 | CA | ASP | A | 837 | 93.875 | 66.451 | 82.199 | 1.00 | 29.32 | 6 |
| ATOM | 4337 | CB | ASP | A | 837 | 95.076 | 66.585 | 81.293 | 1.00 | 30.00 | 6 |
| ATOM | 4338 | CG | ASP | A | 837 | 94.639 | 66.703 | 79.857 | 1.00 | 31.07 | 6 |
| ATOM | 4339 | OD1 | ASP | A | 837 | 93.446 | 66.479 | 79.575 | 1.00 | 28.53 | 8 |
| ATOM | 4340 | OD2 | ASP | A | 837 | 95.498 | 67.029 | 79.005 | 1.00 | 32.80 | 8 |
| ATOM | 4341 | C | ASP | A | 837 | 92.928 | 65.446 | 81.631 | 1.00 | 28.57 | 6 |
| ATOM | 4342 | O | ASP | A | 837 | 93.243 | 64.272 | 81.543 | 1.00 | 29.74 | 8 |
| ATOM | 4343 | N | LEU | A | 838 | 91.799 | 65.899 | 81.263 | 1.00 | 25.47 | 7 |
| ATOM | 4344 | CA | LEU | A | 838 | 90.845 | 64.926 | 80.831 | 1.00 | 26.83 | 6 |
| ATOM | 4345 | CB | LEU | A | 838 | 89.461 | 65.382 | 81.274 | 1.00 | 24.11 | 6 |
| ATOM | 4346 | CG | LEU | A | 838 | 88.917 | 64.823 | 82.594 | 1.00 | 29.24 | 6 |
| ATOM | 4347 | CD1 | LEU | A | 838 | 90.035 | 64.152 | 83.390 | 1.00 | 31.09 | 6 |
| ATOM | 4348 | CD2 | LEU | A | 838 | 88.259 | 65.933 | 83.404 | 1.00 | 27.44 | 6 |
| ATOM | 4349 | C | LEU | A | 838 | 90.853 | 64.640 | 79.353 | 1.00 | 23.12 | 6 |
| ATOM | 4350 | O | LEU | A | 838 | 90.045 | 63.827 | 78.869 | 1.00 | 20.45 | 8 |
| ATOM | 4351 | N | ARG | A | 839 | 91.750 | 65.291 | 78.617 | 1.00 | 23.63 | 7 |
| ATOM | 4352 | CA | ARG | A | 839 | 91.837 | 65.081 | 77.169 | 1.00 | 25.47 | 6 |
| ATOM | 4353 | CB | ARG | A | 839 | 92.722 | 66.137 | 76.511 | 1.00 | 26.36 | 6 |
| ATOM | 4354 | CG | ARG | A | 839 | 92.114 | 67.525 | 76.570 | 1.00 | 30.91 | 6 |
| ATOM | 4355 | CD | ARG | A | 839 | 92.842 | 68.488 | 75.666 | 1.00 | 34.49 | 6 |
| ATOM | 4356 | NE | ARG | A | 839 | 94.173 | 68.001 | 75.323 | 1.00 | 38.70 | 7 |
| ATOM | 4357 | CZ | ARG | A | 839 | 95.060 | 68.696 | 74.618 | 1.00 | 41.12 | 6 |
| ATOM | 4358 | NH1 | ARG | A | 839 | 94.753 | 69.913 | 74.179 | 1.00 | 42.88 | 7 |
| ATOM | 4359 | NH2 | ARG | A | 839 | 96.253 | 68.179 | 74.360 | 1.00 | 41.59 | 7 |
| ATOM | 4360 | C | ARG | A | 839 | 92.338 | 63.682 | 76.875 | 1.00 | 24.92 | 6 |
| ATOM | 4361 | O | ARG | A | 839 | 92.048 | 63.090 | 75.837 | 1.00 | 23.84 | 8 |
| ATOM | 4362 | N | GLN | A | 840 | 93.114 | 63.164 | 77.819 | 1.00 | 25.10 | 7 |
| ATOM | 4363 | CA | GLN | A | 840 | 93.694 | 61.842 | 77.693 | 1.00 | 26.20 | 6 |
| ATOM | 4364 | CB | GLN | A | 840 | 94.846 | 61.649 | 78.689 | 1.00 | 29.04 | 6 |
| ATOM | 4365 | CG | GLN | A | 840 | 95.997 | 62.619 | 78.472 | 1.00 | 35.48 | 6 |
| ATOM | 4366 | CD | GLN | A | 840 | 97.142 | 62.388 | 79.431 | 1.00 | 39.77 | 6 |
| ATOM | 4367 | OE1 | GLN | A | 840 | 97.552 | 61.259 | 79.667 | 1.00 | 44.25 | 8 |
| ATOM | 4368 | NE2 | GLN | A | 840 | 97.810 | 63.309 | 80.094 | 1.00 | 42.92 | 7 |
| ATOM | 4369 | C | GLN | A | 840 | 92.604 | 60.808 | 77.911 | 1.00 | 24.56 | 6 |
| ATOM | 4370 | O | GLN | A | 840 | 92.501 | 59.821 | 77.170 | 1.00 | 25.02 | 8 |
| ATOM | 4371 | N | ASP | A | 841 | 91.783 | 61.041 | 78.929 | 1.00 | 23.42 | 7 |
| ATOM | 4372 | CA | ASP | A | 841 | 90.677 | 60.150 | 79.223 | 1.00 | 23.09 | 6 |
| ATOM | 4373 | CB | ASP | A | 841 | 89.941 | 60.626 | 80.481 | 1.00 | 25.45 | 6 |
| ATOM | 4374 | CG | ASP | A | 841 | 90.538 | 60.045 | 81.780 | 1.00 | 30.39 | 6 |
| ATOM | 4375 | OD1 | ASP | A | 841 | 91.717 | 60.340 | 82.099 | 1.00 | 31.69 | 8 |
| ATOM | 4376 | OD2 | ASP | A | 841 | 89.824 | 59.293 | 82.476 | 1.00 | 30.95 | 8 |
| ATOM | 4377 | C | ASP | A | 841 | 89.720 | 60.126 | 78.014 | 1.00 | 22.44 | 6 |
| ATOM | 4378 | O | ASP | A | 841 | 89.209 | 59.072 | 77.637 | 1.00 | 20.36 | 8 |
| ATOM | 4379 | N | MET | A | 842 | 89.472 | 61.283 | 77.407 | 1.00 | 20.01 | 7 |
| ATOM | 4380 | CA | MET | A | 842 | 88.575 | 61.318 | 76.249 | 1.00 | 21.15 | 6 |
| ATOM | 4381 | CB | MET | A | 842 | 88.410 | 62.741 | 75.720 | 1.00 | 21.76 | 6 |
| ATOM | 4382 | CG | MET | A | 842 | 87.710 | 63.681 | 76.681 | 1.00 | 25.39 | 6 |
| ATOM | 4383 | SD | MET | A | 842 | 87.407 | 65.307 | 75.949 | 1.00 | 29.43 | 16 |
| ATOM | 4384 | CE | MET | A | 842 | 88.996 | 65.658 | 75.209 | 1.00 | 28.32 | 6 |
| ATOM | 4385 | C | MET | A | 842 | 89.098 | 60.424 | 75.126 | 1.00 | 21.60 | 6 |
| ATOM | 4386 | O | MET | A | 842 | 88.331 | 59.684 | 74.516 | 1.00 | 23.11 | 8 |
| ATOM | 4387 | N | LEU | A | 843 | 90.404 | 60.494 | 74.881 | 1.00 | 20.78 | 7 |
| ATOM | 4388 | CA | LEU | A | 843 | 91.048 | 59.704 | 73.843 | 1.00 | 21.23 | 6 |
| ATOM | 4389 | CB | LEU | A | 843 | 92.516 | 60.114 | 73.694 | 1.00 | 21.07 | 6 |
| ATOM | 4390 | CG | LEU | A | 843 | 92.916 | 61.142 | 72.612 | 1.00 | 26.77 | 6 |
| ATOM | 4391 | CD1 | LEU | A | 843 | 91.671 | 61.713 | 71.933 | 1.00 | 24.36 | 6 |
| ATOM | 4392 | CD2 | LEU | A | 843 | 93.752 | 62.251 | 73.210 | 1.00 | 23.36 | 6 |
| ATOM | 4393 | C | LEU | A | 843 | 90.942 | 58.215 | 74.127 | 1.00 | 21.17 | 6 |
| ATOM | 4394 | O | LEU | A | 843 | 90.652 | 57.449 | 73.217 | 1.00 | 18.83 | 8 |
| ATOM | 4395 | N | ILE | A | 844 | 91.175 | 57.799 | 75.379 | 1.00 | 21.07 | 7 |
| ATOM | 4396 | CA | ILE | A | 844 | 91.154 | 56.381 | 75.693 | 1.00 | 20.34 | 6 |
| ATOM | 4397 | CB | ILE | A | 844 | 91.709 | 56.119 | 77.117 | 1.00 | 20.04 | 6 |
| ATOM | 4398 | CG2 | ILE | A | 844 | 91.618 | 54.642 | 77.469 | 1.00 | 19.69 | 6 |
| ATOM | 4399 | CG1 | ILE | A | 844 | 93.161 | 56.604 | 77.212 | 1.00 | 20.78 | 6 |
| ATOM | 4400 | CD1 | ILE | A | 844 | 94.171 | 55.631 | 76.634 | 1.00 | 27.44 | 6 |
| ATOM | 4401 | C | ILE | A | 844 | 89.743 | 55.739 | 75.577 | 1.00 | 21.15 | 6 |
| ATOM | 4402 | O | ILE | A | 844 | 89.618 | 54.616 | 75.083 | 1.00 | 21.10 | 8 |
| ATOM | 4403 | N | LEU | A | 845 | 88.670 | 56.431 | 76.006 | 1.00 | 20.55 | 7 |
| ATOM | 4404 | CA | LEU | A | 845 | 87.277 | 55.903 | 75.926 | 1.00 | 23.92 | 6 |
| ATOM | 4405 | CB | LEU | A | 845 | 86.299 | 56.926 | 76.535 | 1.00 | 23.17 | 6 |
| ATOM | 4406 | CG | LEU | A | 845 | 85.670 | 56.624 | 77.890 | 1.00 | 27.74 | 6 |
| ATOM | 4407 | CD1 | LEU | A | 845 | 86.482 | 55.575 | 78.649 | 1.00 | 27.88 | 6 |
| ATOM | 4408 | CD2 | LEU | A | 845 | 85.529 | 57.897 | 78.698 | 1.00 | 21.27 | 6 |
| ATOM | 4409 | C | LEU | A | 845 | 86.852 | 55.680 | 74.497 | 1.00 | 23.24 | 6 |
| ATOM | 4410 | O | LEU | A | 845 | 86.360 | 54.608 | 74.112 | 1.00 | 22.70 | 8 |
| ATOM | 4411 | N | GLN | A | 846 | 87.077 | 56.728 | 73.741 | 1.00 | 22.52 | 7 |
| ATOM | 4412 | CA | GLN | A | 846 | 86.717 | 56.675 | 72.370 | 1.00 | 24.33 | 6 |
| ATOM | 4413 | CB | GLN | A | 846 | 87.069 | 57.959 | 71.655 | 1.00 | 25.76 | 6 |
| ATOM | 4414 | CG | GLN | A | 846 | 86.222 | 58.180 | 70.422 | 1.00 | 31.35 | 6 |
| ATOM | 4415 | CD | GLN | A | 846 | 84.741 | 57.988 | 70.738 | 1.00 | 35.35 | 6 |
| ATOM | 4416 | OE1 | GLN | A | 846 | 84.140 | 58.754 | 71.500 | 1.00 | 38.19 | 8 |
| ATOM | 4417 | NE2 | GLN | A | 846 | 83.951 | 57.030 | 70.263 | 1.00 | 37.33 | 7 |
| ATOM | 4418 | C | GLN | A | 846 | 87.393 | 55.504 | 71.689 | 1.00 | 23.13 | 6 |
| ATOM | 4419 | O | GLN | A | 846 | 86.754 | 54.835 | 70.872 | 1.00 | 24.23 | 8 |
| ATOM | 4420 | N | ILE | A | 847 | 88.661 | 55.228 | 72.016 | 1.00 | 23.30 | 7 |
| ATOM | 4421 | CA | ILE | A | 847 | 89.302 | 54.081 | 71.394 | 1.00 | 25.24 | 6 |
| ATOM | 4422 | CB | ILE | A | 847 | 90.805 | 54.025 | 71.696 | 1.00 | 24.57 | 6 |
| ATOM | 4423 | CG2 | ILE | A | 847 | 91.401 | 52.714 | 71.223 | 1.00 | 24.69 | 6 |
| ATOM | 4424 | CG1 | ILE | A | 847 | 91.519 | 55.198 | 71.021 | 1.00 | 26.75 | 6 |
| ATOM | 4425 | CD1 | ILE | A | 847 | 93.023 | 55.167 | 71.171 | 1.00 | 27.11 | 6 |
| ATOM | 4426 | C | ILE | A | 847 | 88.590 | 52.847 | 71.917 | 1.00 | 25.46 | 6 |
| ATOM | 4427 | O | ILE | A | 847 | 88.419 | 51.847 | 71.223 | 1.00 | 26.70 | 8 |
| ATOM | 4428 | N | LEU | A | 848 | 88.162 | 52.937 | 73.174 | 1.00 | 24.23 | 7 |
| ATOM | 4429 | CA | LEU | A | 848 | 87.438 | 51.842 | 73.813 | 1.00 | 24.46 | 6 |
| ATOM | 4430 | CB | LEU | A | 848 | 87.125 | 52.192 | 75.277 | 1.00 | 23.96 | 6 |
| ATOM | 4431 | CG | LEU | A | 848 | 87.220 | 51.087 | 76.366 | 1.00 | 26.12 | 6 |
| ATOM | 4432 | CD1 | LEU | A | 848 | 88.228 | 50.020 | 75.972 | 1.00 | 22.04 | 6 |
| ATOM | 4433 | CD2 | LEU | A | 848 | 87.601 | 51.699 | 77.701 | 1.00 | 22.56 | 6 |
| ATOM | 4434 | C | LEU | A | 848 | 86.128 | 51.609 | 73.053 | 1.00 | 21.24 | 6 |
| ATOM | 4435 | O | LEU | A | 848 | 85.735 | 50.469 | 72.800 | 1.00 | 19.25 | 8 |
| ATOM | 4436 | N | ARG | A | 849 | 85.451 | 52.687 | 72.676 | 1.00 | 22.38 | 7 |
| ATOM | 4437 | CA | ARG | A | 849 | 84.207 | 52.541 | 71.932 | 1.00 | 21.46 | 6 |
| ATOM | 4438 | CB | ARG | A | 849 | 83.490 | 53.889 | 71.816 | 1.00 | 25.04 | 6 |
| ATOM | 4439 | CG | ARG | A | 849 | 82.986 | 54.429 | 73.165 | 1.00 | 29.55 | 6 |
| ATOM | 4440 | CD | ARG | A | 849 | 82.241 | 55.762 | 73.017 | 1.00 | 33.96 | 6 |
| ATOM | 4441 | NE | ARG | A | 849 | 80.955 | 55.762 | 73.718 | 1.00 | 36.92 | 7 |
| ATOM | 4442 | CZ | ARG | A | 849 | 80.654 | 56.543 | 74.754 | 1.00 | 38.79 | 6 |
| ATOM | 4443 | NH1 | ARG | A | 849 | 81.545 | 57.407 | 75.227 | 1.00 | 37.36 | 7 |
| ATOM | 4444 | NH2 | ARG | A | 849 | 79.452 | 56.460 | 75.324 | 1.00 | 39.00 | 7 |
| ATOM | 4445 | C | ARG | A | 849 | 84.497 | 51.948 | 70.552 | 1.00 | 22.58 | 6 |
| ATOM | 4446 | O | ARG | A | 849 | 83.683 | 51.196 | 69.997 | 1.00 | 23.09 | 8 |
| ATOM | 4447 | N | ILE | A | 850 | 85.662 | 52.264 | 69.998 | 1.00 | 21.39 | 7 |
| ATOM | 4448 | CA | ILE | A | 850 | 86.018 | 51.718 | 68.701 | 1.00 | 24.04 | 6 |
| ATOM | 4449 | CB | ILE | A | 850 | 87.279 | 52.385 | 68.155 | 1.00 | 26.95 | 6 |
| ATOM | 4450 | CG2 | ILE | A | 850 | 87.798 | 51.608 | 66.961 | 1.00 | 23.93 | 6 |
| ATOM | 4451 | CG1 | ILE | A | 850 | 86.955 | 53.841 | 67.793 | 1.00 | 28.31 | 6 |
| ATOM | 4452 | CD1 | ILE | A | 850 | 88.136 | 54.626 | 67.313 | 1.00 | 28.18 | 6 |
| ATOM | 4453 | C | ILE | A | 850 | 86.248 | 50.213 | 68.839 | 1.00 | 25.30 | 6 |
| ATOM | 4454 | O | ILE | A | 850 | 85.786 | 49.418 | 68.001 | 1.00 | 22.28 | 8 |
| ATOM | 4455 | N | MET | A | 851 | 86.952 | 49.814 | 69.895 | 1.00 | 22.18 | 7 |
| ATOM | 4456 | CA | MET | A | 851 | 87.200 | 48.394 | 70.100 | 1.00 | 25.20 | 6 |
| ATOM | 4457 | CB | MET | A | 851 | 88.085 | 48.160 | 71.320 | 1.00 | 22.80 | 6 |
| ATOM | 4458 | CG | MET | A | 851 | 89.511 | 48.642 | 71.126 | 1.00 | 24.85 | 6 |
| ATOM | 4459 | SD | MET | A | 851 | 90.560 | 48.261 | 72.553 | 1.00 | 25.62 | 16 |
| ATOM | 4460 | CE | MET | A | 851 | 89.680 | 49.062 | 73.762 | 1.00 | 32.45 | 6 |
| ATOM | 4461 | C | MET | A | 851 | 85.895 | 47.626 | 70.233 | 1.00 | 27.14 | 6 |
| ATOM | 4462 | O | MET | A | 851 | 85.814 | 46.462 | 69.828 | 1.00 | 28.81 | 8 |
| ATOM | 4463 | N | GLU | A | 852 | 84.877 | 48.278 | 70.793 | 1.00 | 28.75 | 7 |
| ATOM | 4464 | CA | GLU | A | 852 | 83.562 | 47.665 | 70.941 | 1.00 | 29.10 | 6 |
| ATOM | 4465 | CB | GLU | A | 852 | 82.590 | 48.580 | 71.695 | 1.00 | 31.35 | 6 |
| ATOM | 4466 | CG | GLU | A | 852 | 81.937 | 48.011 | 72.937 | 1.00 | 37.95 | 6 |
| ATOM | 4467 | CD | GLU | A | 852 | 80.894 | 48.972 | 73.510 | 1.00 | 40.62 | 6 |
| ATOM | 4468 | OE1 | GLU | A | 852 | 81.211 | 50.173 | 73.696 | 1.00 | 43.21 | 8 |
| ATOM | 4469 | OE2 | GLU | A | 852 | 79.758 | 48.531 | 73.777 | 1.00 | 40.46 | 8 |
| ATOM | 4470 | C | GLU | A | 852 | 82.960 | 47.401 | 69.566 | 1.00 | 28.89 | 6 |
| ATOM | 4471 | O | GLU | A | 852 | 82.405 | 46.323 | 69.319 | 1.00 | 24.73 | 8 |
| ATOM | 4472 | N | SER | A | 853 | 83.037 | 48.396 | 68.681 | 1.00 | 28.85 | 7 |
| ATOM | 4473 | CA | SER | A | 853 | 82.473 | 48.242 | 67.348 | 1.00 | 31.66 | 6 |
| ATOM | 4474 | CB | SER | A | 853 | 82.587 | 49.543 | 66.546 | 1.00 | 31.63 | 6 |
| ATOM | 4475 | OG | SER | A | 853 | 83.931 | 49.853 | 66.230 | 1.00 | 39.87 | 8 |
| ATOM | 4476 | C | SER | A | 853 | 83.191 | 47.101 | 66.639 | 1.00 | 31.82 | 6 |
| ATOM | 4477 | O | SER | A | 853 | 82.560 | 46.283 | 65.979 | 1.00 | 34.71 | 8 |
| ATOM | 4478 | N | ILE | A | 854 | 84.507 | 47.041 | 66.791 | 1.00 | 32.37 | 7 |
| ATOM | 4479 | CA | ILE | A | 854 | 85.281 | 45.977 | 66.180 | 1.00 | 31.21 | 6 |
| ATOM | 4480 | CB | ILE | A | 854 | 86.780 | 46.097 | 66.525 | 1.00 | 31.90 | 6 |
| ATOM | 4481 | CG2 | ILE | A | 854 | 87.506 | 44.807 | 66.169 | 1.00 | 28.76 | 6 |
| ATOM | 4482 | CG1 | ILE | A | 854 | 87.392 | 47.290 | 65.779 | 1.00 | 30.08 | 6 |
| ATOM | 4483 | CD1 | ILE | A | 854 | 88.807 | 47.605 | 66.201 | 1.00 | 30.97 | 6 |
| ATOM | 4484 | C | ILE | A | 854 | 84.757 | 44.634 | 66.682 | 1.00 | 34.17 | 6 |
| ATOM | 4485 | O | ILE | A | 854 | 84.441 | 43.765 | 65.860 | 1.00 | 33.61 | 8 |
| ATOM | 4486 | N | TRP | A | 855 | 84.653 | 44.463 | 67.998 | 1.00 | 33.62 | 7 |
| ATOM | 4487 | CA | TRP | A | 855 | 84.201 | 43.199 | 68.537 | 1.00 | 33.75 | 6 |
| ATOM | 4488 | CB | TRP | A | 855 | 84.285 | 43.268 | 70.067 | 1.00 | 30.90 | 6 |
| ATOM | 4489 | CG | TRP | A | 855 | 85.706 | 43.114 | 70.542 | 1.00 | 29.06 | 6 |
| ATOM | 4490 | CD2 | TRP | A | 855 | 86.264 | 43.697 | 71.730 | 1.00 | 28.99 | 6 |
| ATOM | 4491 | CE2 | TRP | A | 855 | 87.616 | 43.297 | 71.794 | 1.00 | 25.88 | 6 |
| ATOM | 4492 | CE3 | TRP | A | 855 | 85.749 | 44.515 | 72.748 | 1.00 | 28.49 | 6 |
| ATOM | 4493 | CD1 | TRP | A | 855 | 86.715 | 42.410 | 69.953 | 1.00 | 27.52 | 6 |
| ATOM | 4494 | NE1 | TRP | A | 855 | 87.864 | 42.515 | 70.699 | 1.00 | 28.53 | 7 |
| ATOM | 4495 | CZ2 | TRP | A | 855 | 88.468 | 43.684 | 72.836 | 1.00 | 27.73 | 6 |
| ATOM | 4496 | CZ3 | TRP | A | 855 | 86.597 | 44.899 | 73.790 | 1.00 | 30.89 | 6 |
| ATOM | 4497 | CH2 | TRP | A | 855 | 87.945 | 44.480 | 73.822 | 1.00 | 28.83 | 6 |
| ATOM | 4498 | C | TRP | A | 855 | 82.793 | 42.718 | 68.091 | 1.00 | 34.39 | 6 |
| ATOM | 4499 | O | TRP | A | 855 | 82.523 | 41.521 | 68.221 | 1.00 | 32.96 | 8 |
| ATOM | 4500 | N | GLU | A | 856 | 81.900 | 43.590 | 67.587 | 1.00 | 36.17 | 7 |
| ATOM | 4501 | CA | GLU | A | 856 | 80.543 | 43.165 | 67.201 | 1.00 | 39.23 | 6 |
| ATOM | 4502 | CB | GLU | A | 856 | 79.529 | 44.270 | 67.470 | 1.00 | 41.22 | 6 |
| ATOM | 4503 | CG | GLU | A | 856 | 78.358 | 44.240 | 66.499 | 1.00 | 44.51 | 6 |
| ATOM | 4504 | CD | GLU | A | 856 | 77.608 | 45.562 | 66.473 | 1.00 | 48.79 | 6 |
| ATOM | 4505 | OE1 | GLU | A | 856 | 77.931 | 46.419 | 65.624 | 1.00 | 51.30 | 8 |
| ATOM | 4506 | OE2 | GLU | A | 856 | 76.697 | 45.748 | 67.308 | 1.00 | 50.83 | 8 |
| ATOM | 4507 | C | GLU | A | 856 | 80.463 | 42.710 | 65.751 | 1.00 | 39.46 | 6 |
| ATOM | 4508 | O | GLU | A | 856 | 79.469 | 42.143 | 65.313 | 1.00 | 40.68 | 8 |
| ATOM | 4509 | N | THR | A | 857 | 81.525 | 42.964 | 64.980 | 1.00 | 41.39 | 7 |
| ATOM | 4510 | CA | THR | A | 857 | 81.624 | 42.468 | 63.607 | 1.00 | 41.68 | 6 |
| ATOM | 4511 | CB | THR | A | 857 | 82.863 | 42.928 | 62.836 | 1.00 | 41.15 | 6 |
| ATOM | 4512 | OG1 | THR | A | 857 | 84.046 | 42.581 | 63.558 | 1.00 | 40.91 | 8 |
| ATOM | 4513 | CG2 | THR | A | 857 | 82.825 | 44.418 | 62.612 | 1.00 | 41.66 | 6 |
| ATOM | 4514 | C | THR | A | 857 | 81.906 | 41.007 | 63.725 | 1.00 | 43.53 | 6 |
| ATOM | 4515 | O | THR | A | 857 | 81.746 | 40.210 | 62.800 | 1.00 | 44.76 | 8 |
| ATOM | 4516 | N | GLU | A | 858 | 82.346 | 40.659 | 64.912 | 1.00 | 43.81 | 7 |
| ATOM | 4517 | CA | GLU | A | 858 | 82.694 | 39.286 | 65.139 | 1.00 | 44.45 | 6 |
| ATOM | 4518 | CB | GLU | A | 858 | 84.187 | 39.211 | 65.484 | 1.00 | 44.95 | 6 |
| ATOM | 4519 | CG | GLU | A | 858 | 85.028 | 39.890 | 64.414 | 1.00 | 48.01 | 6 |
| ATOM | 4520 | CD | GLU | A | 858 | 86.359 | 39.203 | 64.207 | 1.00 | 50.19 | 6 |
| ATOM | 4521 | OE1 | GLU | A | 858 | 87.146 | 39.653 | 63.362 | 1.00 | 50.25 | 8 |
| ATOM | 4522 | OE2 | GLU | A | 858 | 86.614 | 38.200 | 64.920 | 1.00 | 51.36 | 8 |
| ATOM | 4523 | C | GLU | A | 858 | 81.755 | 38.637 | 66.140 | 1.00 | 44.35 | 6 |
| ATOM | 4524 | O | GLU | A | 858 | 82.129 | 37.729 | 66.899 | 1.00 | 45.05 | 8 |
| ATOM | 4525 | N | SER | A | 859 | 80.507 | 39.142 | 66.131 | 1.00 | 43.64 | 7 |
| ATOM | 4526 | CA | SER | A | 859 | 79.463 | 38.661 | 67.010 | 1.00 | 44.87 | 6 |
| ATOM | 4527 | CB | SER | A | 859 | 79.025 | 37.241 | 66.662 | 1.00 | 46.73 | 6 |
| ATOM | 4528 | OG | SER | A | 859 | 78.627 | 37.137 | 65.305 | 1.00 | 49.76 | 8 |
| ATOM | 4529 | C | SER | A | 859 | 80.000 | 38.665 | 68.424 | 1.00 | 44.35 | 6 |
| ATOM | 4530 | O | SER | A | 859 | 80.133 | 37.617 | 69.062 | 1.00 | 42.84 | 8 |
| ATOM | 4531 | N | LEU | A | 860 | 80.352 | 39.819 | 68.869 | 1.00 | 43.06 | 7 |
| ATOM | 4532 | CA | LEU | A | 860 | 80.864 | 39.787 | 70.167 | 1.00 | 41.98 | 6 |
| ATOM | 4533 | CB | LEU | A | 860 | 82.395 | 39.619 | 70.132 | 1.00 | 44.00 | 6 |
| ATOM | 4534 | CG | LEU | A | 860 | 82.937 | 38.323 | 69.472 | 1.00 | 45.82 | 6 |
| ATOM | 4535 | CD1 | LEU | A | 860 | 84.459 | 38.395 | 69.296 | 1.00 | 44.32 | 6 |
| ATOM | 4536 | CD2 | LEU | A | 860 | 82.547 | 37.103 | 70.299 | 1.00 | 45.98 | 6 |
| ATOM | 4537 | C | LEU | A | 860 | 80.400 | 40.992 | 70.887 | 1.00 | 40.30 | 6 |
| ATOM | 4538 | O | LEU | A | 860 | 80.358 | 42.111 | 70.390 | 1.00 | 35.80 | 8 |
| ATOM | 4539 | N | ASP | A | 861 | 80.042 | 40.692 | 72.077 | 1.00 | 37.98 | 7 |
| ATOM | 4540 | CA | ASP | A | 861 | 79.640 | 41.628 | 73.049 | 1.00 | 36.81 | 6 |
| ATOM | 4541 | CB | ASP | A | 861 | 78.150 | 41.579 | 73.331 | 1.00 | 38.21 | 6 |
| ATOM | 4542 | CG | ASP | A | 861 | 77.525 | 42.929 | 73.121 | 1.00 | 37.96 | 6 |
| ATOM | 4543 | OD1 | ASP | A | 861 | 78.226 | 43.842 | 72.637 | 1.00 | 35.38 | 8 |
| ATOM | 4544 | OD2 | ASP | A | 861 | 76.326 | 43.099 | 73.427 | 1.00 | 39.83 | 8 |
| ATOM | 4545 | C | ASP | A | 861 | 80.436 | 41.284 | 74.283 | 1.00 | 36.84 | 6 |
| ATOM | 4546 | O | ASP | A | 861 | 80.454 | 40.155 | 74.756 | 1.00 | 36.77 | 8 |
| ATOM | 4547 | N | LEU | A | 862 | 81.085 | 42.278 | 74.808 | 1.00 | 33.55 | 7 |
| ATOM | 4548 | CA | LEU | A | 862 | 81.904 | 42.038 | 75.975 | 1.00 | 31.00 | 6 |
| ATOM | 4549 | CB | LEU | A | 862 | 83.328 | 42.438 | 75.639 | 1.00 | 30.05 | 6 |
| ATOM | 4550 | CG | LEU | A | 862 | 83.896 | 41.557 | 74.539 | 1.00 | 32.50 | 6 |
| ATOM | 4551 | CD1 | LEU | A | 862 | 84.710 | 40.420 | 75.141 | 1.00 | 29.68 | 6 |
| ATOM | 4552 | CD2 | LEU | A | 862 | 82.769 | 41.006 | 73.660 | 1.00 | 33.90 | 6 |
| ATOM | 4553 | C | LEU | A | 862 | 81.342 | 42.731 | 77.193 | 1.00 | 30.43 | 6 |
| ATOM | 4554 | O | LEU | A | 862 | 81.368 | 42.203 | 78.305 | 1.00 | 29.81 | 8 |
| ATOM | 4555 | N | CYS | A | 863 | 80.815 | 43.935 | 76.948 | 1.00 | 28.33 | 7 |
| ATOM | 4556 | CA | CYS | A | 863 | 80.122 | 44.778 | 77.926 | 1.00 | 28.97 | 6 |
| ATOM | 4557 | CB | CYS | A | 863 | 79.391 | 43.874 | 78.917 | 1.00 | 28.01 | 6 |
| ATOM | 4558 | SG | CYS | A | 863 | 78.316 | 42.621 | 78.152 | 1.00 | 34.15 | 16 |
| ATOM | 4559 | C | CYS | A | 863 | 81.041 | 45.821 | 78.609 | 1.00 | 27.73 | 6 |
| ATOM | 4560 | O | CYS | A | 863 | 80.903 | 46.120 | 79.803 | 1.00 | 29.68 | 8 |
| ATOM | 4561 | N | LEU | A | 864 | 81.966 | 46.366 | 77.801 | 1.00 | 23.28 | 7 |
| ATOM | 4562 | CA | LEU | A | 864 | 82.878 | 47.410 | 78.300 | 1.00 | 22.60 | 6 |
| ATOM | 4563 | CB | LEU | A | 864 | 83.802 | 47.917 | 77.188 | 1.00 | 20.65 | 6 |
| ATOM | 4564 | CG | LEU | A | 864 | 84.125 | 46.896 | 76.070 | 1.00 | 23.75 | 6 |
| ATOM | 4565 | CD1 | LEU | A | 864 | 85.202 | 47.445 | 75.140 | 1.00 | 24.03 | 6 |
| ATOM | 4566 | CD2 | LEU | A | 864 | 84.568 | 45.571 | 76.677 | 1.00 | 19.96 | 6 |
| ATOM | 4567 | C | LEU | A | 864 | 81.979 | 48.491 | 78.835 | 1.00 | 22.64 | 6 |
| ATOM | 4568 | O | LEU | A | 864 | 80.763 | 48.413 | 78.652 | 1.00 | 22.47 | 8 |
| ATOM | 4569 | N | LEU | A | 865 | 82.516 | 49.504 | 79.486 | 1.00 | 24.08 | 7 |
| ATOM | 4570 | CA | LEU | A | 865 | 81.559 | 50.462 | 80.000 | 1.00 | 27.29 | 6 |
| ATOM | 4571 | CB | LEU | A | 865 | 81.187 | 50.097 | 81.414 | 1.00 | 28.60 | 6 |
| ATOM | 4572 | CG | LEU | A | 865 | 79.909 | 50.704 | 81.919 | 1.00 | 33.50 | 6 |
| ATOM | 4573 | CD1 | LEU | A | 865 | 78.757 | 50.385 | 80.978 | 1.00 | 35.81 | 6 |
| ATOM | 4574 | CD2 | LEU | A | 865 | 79.609 | 50.214 | 83.325 | 1.00 | 31.76 | 6 |
| ATOM | 4575 | C | LEU | A | 865 | 82.055 | 51.879 | 79.907 | 1.00 | 27.15 | 6 |
| ATOM | 4576 | O | LEU | A | 865 | 82.392 | 52.574 | 80.877 | 1.00 | 28.70 | 8 |
| ATOM | 4577 | N | PRO | A | 866 | 82.076 | 52.246 | 78.634 | 1.00 | 28.02 | 7 |
| ATOM | 4578 | CD | PRO | A | 866 | 82.583 | 51.235 | 77.717 | 1.00 | 27.98 | 6 |
| ATOM | 4579 | CA | PRO | A | 866 | 82.534 | 53.558 | 78.253 | 1.00 | 26.93 | 6 |
| ATOM | 4580 | CB | PRO | A | 866 | 82.466 | 53.450 | 76.728 | 1.00 | 27.28 | 6 |
| ATOM | 4581 | CG | PRO | A | 866 | 82.897 | 52.027 | 76.496 | 1.00 | 29.48 | 6 |
| ATOM | 4582 | C | PRO | A | 866 | 81.803 | 54.681 | 78.948 | 1.00 | 25.41 | 6 |
| ATOM | 4583 | O | PRO | A | 866 | 80.751 | 55.082 | 78.448 | 1.00 | 26.68 | 8 |
| ATOM | 4584 | N | TYR | A | 867 | 82.270 | 55.213 | 80.036 | 1.00 | 24.65 | 7 |
| ATOM | 4585 | CA | TYR | A | 867 | 81.483 | 56.259 | 80.657 | 1.00 | 23.67 | 6 |
| ATOM | 4586 | CB | TYR | A | 867 | 81.701 | 56.190 | 82.167 | 1.00 | 22.27 | 6 |
| ATOM | 4587 | CG | TYR | A | 867 | 83.153 | 56.256 | 82.477 | 1.00 | 20.52 | 6 |
| ATOM | 4588 | CD1 | TYR | A | 867 | 83.735 | 57.506 | 82.658 | 1.00 | 20.96 | 6 |
| ATOM | 4589 | CE1 | TYR | A | 867 | 85.086 | 57.631 | 82.962 | 1.00 | 22.88 | 6 |
| ATOM | 4590 | CD2 | TYR | A | 867 | 83.959 | 55.125 | 82.610 | 1.00 | 20.74 | 6 |
| ATOM | 4591 | CE2 | TYR | A | 867 | 85.309 | 55.237 | 82.910 | 1.00 | 21.17 | 6 |
| ATOM | 4592 | CZ | TYR | A | 867 | 85.866 | 56.493 | 83.085 | 1.00 | 21.63 | 6 |
| ATOM | 4593 | OH | TYR | A | 867 | 87.205 | 56.614 | 83.373 | 1.00 | 26.21 | 8 |
| ATOM | 4594 | C | TYR | A | 867 | 81.774 | 57.645 | 80.077 | 1.00 | 21.26 | 6 |
| ATOM | 4595 | O | TYR | A | 867 | 82.874 | 57.893 | 79.578 | 1.00 | 19.77 | 8 |
| ATOM | 4596 | N | GLY | A | 868 | 80.799 | 58.568 | 80.159 | 1.00 | 23.51 | 7 |
| ATOM | 4597 | CA | GLY | A | 868 | 80.932 | 59.912 | 79.593 | 1.00 | 22.67 | 6 |
| ATOM | 4598 | C | GLY | A | 868 | 82.032 | 60.797 | 80.237 | 1.00 | 23.18 | 6 |
| ATOM | 4599 | O | GLY | A | 868 | 82.191 | 60.782 | 81.457 | 1.00 | 21.51 | 8 |
| ATOM | 4600 | N | CYS | A | 869 | 82.769 | 61.550 | 79.419 | 1.00 | 21.35 | 7 |
| ATOM | 4601 | CA | CYS | A | 869 | 83.852 | 62.388 | 79.930 | 1.00 | 21.65 | 6 |
| ATOM | 4602 | CB | CYS | A | 869 | 85.106 | 61.546 | 80.200 | 1.00 | 22.44 | 6 |
| ATOM | 4603 | SG | CYS | A | 869 | 86.515 | 62.525 | 80.791 | 1.00 | 29.86 | 16 |
| ATOM | 4604 | C | CYS | A | 869 | 84.178 | 63.494 | 78.955 | 1.00 | 23.04 | 6 |
| ATOM | 4605 | O | CYS | A | 869 | 84.322 | 63.259 | 77.761 | 1.00 | 22.26 | 8 |
| ATOM | 4606 | N | ILE | A | 870 | 84.281 | 64.714 | 79.458 | 1.00 | 22.74 | 7 |
| ATOM | 4607 | CA | ILE | A | 870 | 84.577 | 65.840 | 78.590 | 1.00 | 22.94 | 6 |
| ATOM | 4608 | CB | ILE | A | 870 | 83.271 | 66.542 | 78.140 | 1.00 | 27.36 | 6 |
| ATOM | 4609 | CG2 | ILE | A | 870 | 83.565 | 67.913 | 77.544 | 1.00 | 20.34 | 6 |
| ATOM | 4610 | CG1 | ILE | A | 870 | 82.549 | 65.669 | 77.112 | 1.00 | 27.35 | 6 |
| ATOM | 4611 | CD1 | ILE | A | 870 | 82.251 | 66.365 | 75.802 | 1.00 | 27.18 | 6 |
| ATOM | 4612 | C | ILE | A | 870 | 85.479 | 66.848 | 79.263 | 1.00 | 22.69 | 6 |
| ATOM | 4613 | O | ILE | A | 870 | 85.131 | 67.404 | 80.305 | 1.00 | 22.78 | 8 |
| ATOM | 4614 | N | SER | A | 871 | 86.648 | 67.064 | 78.670 | 1.00 | 21.95 | 7 |
| ATOM | 4615 | CA | SER | A | 871 | 87.566 | 68.037 | 79.215 | 1.00 | 20.58 | 6 |
| ATOM | 4616 | CB | SER | A | 871 | 88.974 | 67.826 | 78.670 | 1.00 | 20.62 | 6 |
| ATOM | 4617 | OG | SER | A | 871 | 89.820 | 68.909 | 79.013 | 1.00 | 21.45 | 8 |
| ATOM | 4618 | C | SER | A | 871 | 87.025 | 69.443 | 78.854 | 1.00 | 25.30 | 6 |
| ATOM | 4619 | O | SER | A | 871 | 86.885 | 69.733 | 77.665 | 1.00 | 22.73 | 8 |
| ATOM | 4620 | N | THR | A | 872 | 86.719 | 70.343 | 79.805 | 1.00 | 24.62 | 7 |
| ATOM | 4621 | CA | THR | A | 872 | 86.220 | 71.682 | 79.393 | 1.00 | 24.25 | 6 |
| ATOM | 4622 | CB | THR | A | 872 | 84.936 | 72.067 | 80.130 | 1.00 | 25.46 | 6 |
| ATOM | 4623 | OG1 | THR | A | 872 | 85.153 | 71.958 | 81.542 | 1.00 | 24.15 | 8 |
| ATOM | 4624 | CG2 | THR | A | 872 | 83.789 | 71.159 | 79.708 | 1.00 | 24.80 | 6 |
| ATOM | 4625 | C | THR | A | 872 | 87.313 | 72.764 | 79.581 | 1.00 | 26.84 | 6 |
| ATOM | 4626 | O | THR | A | 872 | 87.098 | 73.945 | 79.246 | 1.00 | 26.62 | 8 |
| ATOM | 4627 | N | GLY | A | 873 | 88.503 | 72.371 | 80.114 | 1.00 | 24.87 | 7 |
| ATOM | 4628 | CA | GLY | A | 873 | 89.609 | 73.311 | 80.346 | 1.00 | 27.57 | 6 |
| ATOM | 4629 | C | GLY | A | 873 | 90.950 | 72.696 | 80.781 | 1.00 | 32.09 | 6 |
| ATOM | 4630 | O | GLY | A | 873 | 91.085 | 71.469 | 80.863 | 1.00 | 32.02 | 8 |
| ATOM | 4631 | N | ASP | A | 874 | 91.954 | 73.568 | 81.060 | 1.00 | 32.51 | 7 |
| ATOM | 4632 | CA | ASP | A | 874 | 93.277 | 73.100 | 81.466 | 1.00 | 35.68 | 6 |
| ATOM | 4633 | CB | ASP | A | 874 | 94.257 | 74.222 | 81.780 | 1.00 | 39.63 | 6 |
| ATOM | 4634 | CG | ASP | A | 874 | 95.533 | 73.741 | 82.508 | 1.00 | 43.33 | 6 |
| ATOM | 4635 | OD1 | ASP | A | 874 | 96.218 | 72.845 | 81.974 | 1.00 | 44.81 | 8 |
| ATOM | 4636 | OD2 | ASP | A | 874 | 95.818 | 74.276 | 83.598 | 1.00 | 48.04 | 8 |
| ATOM | 4637 | C | ASP | A | 874 | 93.199 | 72.255 | 82.696 | 1.00 | 34.44 | 6 |
| ATOM | 4638 | O | ASP | A | 874 | 93.555 | 72.680 | 83.803 | 1.00 | 32.36 | 8 |
| ATOM | 4639 | N | LYS | A | 875 | 92.741 | 71.030 | 82.506 | 1.00 | 34.07 | 7 |
| ATOM | 4640 | CA | LYS | A | 875 | 92.664 | 70.191 | 83.656 | 1.00 | 34.58 | 6 |
| ATOM | 4641 | CB | LYS | A | 875 | 93.744 | 70.581 | 84.661 | 1.00 | 37.54 | 6 |
| ATOM | 4642 | CG | LYS | A | 875 | 95.151 | 70.297 | 84.167 | 1.00 | 41.83 | 6 |
| ATOM | 4643 | CD | LYS | A | 875 | 96.186 | 70.809 | 85.137 | 1.00 | 45.28 | 6 |
| ATOM | 4644 | CE | LYS | A | 875 | 96.200 | 72.333 | 85.185 | 1.00 | 49.42 | 6 |
| ATOM | 4645 | NZ | LYS | A | 875 | 97.118 | 72.841 | 86.243 | 1.00 | 49.43 | 7 |
| ATOM | 4646 | C | LYS | A | 875 | 91.314 | 70.239 | 84.298 | 1.00 | 32.61 | 6 |
| ATOM | 4647 | O | LYS | A | 875 | 91.103 | 69.561 | 85.307 | 1.00 | 30.76 | 8 |
| ATOM | 4648 | N | ILE | A | 876 | 90.395 | 71.001 | 83.774 | 1.00 | 29.12 | 7 |
| ATOM | 4649 | CA | ILE | A | 876 | 89.117 | 70.933 | 84.406 | 1.00 | 27.36 | 6 |
| ATOM | 4650 | CB | ILE | A | 876 | 88.602 | 72.326 | 84.801 | 1.00 | 27.59 | 6 |
| ATOM | 4651 | CG2 | ILE | A | 876 | 88.572 | 73.226 | 83.564 | 1.00 | 28.62 | 6 |
| ATOM | 4652 | CG1 | ILE | A | 876 | 87.235 | 72.293 | 85.471 | 1.00 | 31.32 | 6 |
| ATOM | 4653 | CD1 | ILE | A | 876 | 86.822 | 73.620 | 86.082 | 1.00 | 36.42 | 6 |
| ATOM | 4654 | C | ILE | A | 876 | 88.165 | 70.247 | 83.461 | 1.00 | 25.67 | 6 |
| ATOM | 4655 | O | ILE | A | 876 | 88.241 | 70.426 | 82.244 | 1.00 | 23.93 | 8 |
| ATOM | 4656 | N | GLY | A | 877 | 87.257 | 69.452 | 83.981 | 1.00 | 26.35 | 7 |
| ATOM | 4657 | CA | GLY | A | 877 | 86.299 | 68.759 | 83.149 | 1.00 | 25.30 | 6 |
| ATOM | 4658 | C | GLY | A | 877 | 85.274 | 68.004 | 83.973 | 1.00 | 25.67 | 6 |
| ATOM | 4659 | O | GLY | A | 877 | 85.358 | 67.959 | 85.210 | 1.00 | 25.77 | 8 |
| ATOM | 4660 | N | MET | A | 878 | 84.308 | 67.419 | 83.266 | 1.00 | 24.50 | 7 |
| ATOM | 4661 | CA | MET | A | 878 | 83.234 | 66.637 | 83.869 | 1.00 | 24.77 | 6 |
| ATOM | 4662 | CB | MET | A | 878 | 81.876 | 67.139 | 83.391 | 1.00 | 27.37 | 6 |
| ATOM | 4663 | CG | MET | A | 878 | 81.682 | 66.983 | 81.881 | 1.00 | 30.59 | 6 |
| ATOM | 4664 | SD | MET | A | 878 | 80.168 | 67.768 | 81.303 | 1.00 | 37.68 | 16 |
| ATOM | 4665 | CE | MET | A | 878 | 80.021 | 69.149 | 82.497 | 1.00 | 33.74 | 6 |
| ATOM | 4666 | C | MET | A | 878 | 83.333 | 65.163 | 83.519 | 1.00 | 24.44 | 6 |
| ATOM | 4667 | O | MET | A | 878 | 83.796 | 64.801 | 82.439 | 1.00 | 22.09 | 8 |
| ATOM | 4668 | N | ILE | A | 879 | 82.852 | 64.331 | 84.435 | 1.00 | 23.81 | 7 |
| ATOM | 4669 | CA | ILE | A | 879 | 82.887 | 62.879 | 84.305 | 1.00 | 25.21 | 6 |
| ATOM | 4670 | CB | ILE | A | 879 | 83.990 | 62.290 | 85.231 | 1.00 | 28.14 | 6 |
| ATOM | 4671 | CG2 | ILE | A | 879 | 83.994 | 60.767 | 85.165 | 1.00 | 24.50 | 6 |
| ATOM | 4672 | CG1 | ILE | A | 879 | 85.356 | 62.856 | 84.828 | 1.00 | 28.36 | 6 |
| ATOM | 4673 | CD1 | ILE | A | 879 | 86.456 | 62.566 | 85.817 | 1.00 | 33.59 | 6 |
| ATOM | 4674 | C | ILE | A | 879 | 81.528 | 62.312 | 84.706 | 1.00 | 25.42 | 6 |
| ATOM | 4675 | O | ILE | A | 879 | 80.940 | 62.729 | 85.706 | 1.00 | 27.54 | 8 |
| ATOM | 4676 | N | GLU | A | 880 | 81.022 | 61.363 | 83.924 | 1.00 | 22.89 | 7 |
| ATOM | 4677 | CA | GLU | A | 880 | 79.737 | 60.746 | 84.220 | 1.00 | 21.66 | 6 |
| ATOM | 4678 | CB | GLU | A | 680 | 79.307 | 59.829 | 83.067 | 1.00 | 23.33 | 6 |
| ATOM | 4679 | CG | GLU | A | 880 | 78.062 | 58.980 | 83.347 | 1.00 | 23.77 | 6 |
| ATOM | 4680 | CD | GLU | A | 880 | 77.625 | 58.123 | 82.142 | 1.00 | 27.52 | 6 |
| ATOM | 4681 | OE1 | GLU | A | 880 | 76.607 | 57.409 | 82.252 | 1.00 | 27.19 | 8 |
| ATOM | 4682 | OE2 | GLU | A | 880 | 78.294 | 58.158 | 81.086 | 1.00 | 26.96 | 8 |
| ATOM | 4683 | C | GLU | A | 880 | 79.817 | 59.916 | 85.490 | 1.00 | 23.74 | 6 |
| ATOM | 4684 | O | GLU | A | 880 | 80.807 | 59.213 | 85.708 | 1.00 | 21.46 | 8 |
| ATOM | 4685 | N | ILE | A | 881 | 78.759 | 59.845 | 86.304 | 1.00 | 23.28 | 7 |
| ATOM | 4686 | CA | ILE | A | 881 | 78.715 | 58.957 | 87.516 | 1.00 | 25.56 | 6 |
| ATOM | 4687 | CB | ILE | A | 881 | 78.047 | 59.726 | 88.701 | 1.00 | 25.02 | 6 |
| ATOM | 4688 | CG2 | ILE | A | 881 | 77.703 | 58.750 | 89.811 | 1.00 | 26.07 | 6 |
| ATOM | 4689 | CG1 | ILE | A | 881 | 78.899 | 60.865 | 89.268 | 1.00 | 24.22 | 6 |
| ATOM | 4690 | CD1 | ILE | A | 881 | 78.331 | 61.474 | 90.533 | 1.00 | 28.10 | 6 |
| ATOM | 4691 | C | ILE | A | 881 | 77.908 | 57.634 | 87.150 | 1.00 | 25.01 | 6 |
| ATOM | 4692 | O | ILE | A | 881 | 76.776 | 57.786 | 86.711 | 1.00 | 23.48 | 8 |
| ATOM | 4693 | N | VAL | A | 882 | 78.399 | 56.353 | 87.261 | 1.00 | 24.51 | 7 |
| ATOM | 4694 | CA | VAL | A | 882 | 77.560 | 55.165 | 86.816 | 1.00 | 28.02 | 6 |
| ATOM | 4695 | CB | VAL | A | 882 | 78.492 | 54.055 | 86.225 | 1.00 | 24.82 | 6 |
| ATOM | 4696 | CG1 | VAL | A | 882 | 77.709 | 52.778 | 85.958 | 1.00 | 26.06 | 6 |
| ATOM | 4697 | CG2 | VAL | A | 882 | 79.162 | 54.544 | 84.953 | 1.00 | 24.28 | 6 |
| ATOM | 4698 | C | VAL | A | 882 | 76.576 | 54.553 | 87.905 | 1.00 | 27.84 | 6 |
| ATOM | 4699 | O | VAL | A | 882 | 77.006 | 54.332 | 89.035 | 1.00 | 29.61 | 8 |
| ATOM | 4700 | N | LYS | A | 883 | 75.235 | 54.292 | 87.557 | 1.00 | 32.08 | 7 |
| ATOM | 4701 | CA | LYS | A | 883 | 74.093 | 53.793 | 88.456 | 1.00 | 34.32 | 6 |
| ATOM | 4702 | CB | LYS | A | 883 | 72.761 | 53.910 | 87.705 | 1.00 | 38.24 | 6 |
| ATOM | 4703 | CG | LYS | A | 883 | 72.848 | 55.031 | 88.183 | 1.00 | 44.77 | 6 |
| ATOM | 4704 | CD | LYS | A | 883 | 72.471 | 56.383 | 87.923 | 1.00 | 47.85 | 6 |
| ATOM | 4705 | CE | LYS | A | 883 | 71.687 | 57.514 | 88.559 | 1.00 | 50.43 | 6 |
| ATOM | 4706 | NZ | LYS | A | 883 | 71.717 | 57.443 | 90.046 | 1.00 | 49.61 | 7 |
| ATOM | 4707 | C | LYS | A | 883 | 74.244 | 52.363 | 89.026 | 1.00 | 33.92 | 6 |
| ATOM | 4708 | O | LYS | A | 883 | 74.729 | 51.462 | 88.333 | 1.00 | 33.00 | 8 |
| ATOM | 4709 | N | ASP | A | 884 | 73.815 | 52.136 | 90.315 | 1.00 | 35.30 | 7 |
| ATOM | 4710 | CA | ASP | A | 884 | 74.044 | 50.861 | 90.987 | 1.00 | 36.94 | 6 |
| ATOM | 4711 | CB | ASP | A | 884 | 72.948 | 49.857 | 90.613 | 1.00 | 39.67 | 6 |
| ATOM | 4712 | CG | ASP | A | 884 | 71.577 | 50.278 | 91.114 | 1.00 | 43.46 | 6 |
| ATOM | 4713 | OD1 | ASP | A | 884 | 71.512 | 51.078 | 92.075 | 1.00 | 44.96 | 8 |
| ATOM | 4714 | OD2 | ASP | A | 884 | 70.565 | 49.799 | 90.558 | 1.00 | 44.13 | 8 |
| ATOM | 4715 | C | ASP | A | 884 | 75.411 | 50.262 | 90.683 | 1.00 | 36.20 | 6 |
| ATOM | 4716 | O | ASP | A | 884 | 75.515 | 49.109 | 90.268 | 1.00 | 37.44 | 8 |
| ATOM | 4717 | N | ALA | A | 885 | 76.455 | 51.052 | 90.902 | 1.00 | 34.91 | 7 |
| ATOM | 4718 | CA | ALA | A | 885 | 77.820 | 50.616 | 90.648 | 1.00 | 33.41 | 6 |
| ATOM | 4719 | CB | ALA | A | 885 | 78.346 | 51.268 | 89.378 | 1.00 | 32.80 | 6 |
| ATOM | 4720 | C | ALA | A | 885 | 78.712 | 50.980 | 91.818 | 1.00 | 32.11 | 6 |
| ATOM | 4721 | O | ALA | A | 885 | 78.555 | 52.036 | 92.426 | 1.00 | 31.46 | 8 |
| ATOM | 4722 | N | THR | A | 886 | 79.648 | 50.099 | 92.133 | 1.00 | 31.43 | 7 |
| ATOM | 4723 | CA | THR | A | 886 | 80.576 | 50.353 | 93.223 | 1.00 | 32.43 | 6 |
| ATOM | 4724 | CB | THR | A | 886 | 80.249 | 49.517 | 94.478 | 1.00 | 34.98 | 6 |
| ATOM | 4725 | OG1 | THR | A | 886 | 78.870 | 49.679 | 94.815 | 1.00 | 39.61 | 8 |
| ATOM | 4726 | CG2 | THR | A | 886 | 81.092 | 49.995 | 95.655 | 1.00 | 36.44 | 6 |
| ATOM | 4727 | C | THR | A | 886 | 81.954 | 49.950 | 92.758 | 1.00 | 31.65 | 6 |
| ATOM | 4728 | O | THR | A | 886 | 82.100 | 49.289 | 91.734 | 1.00 | 31.97 | 8 |
| ATOM | 4729 | N | THR | A | 887 | 82.964 | 50.351 | 93.518 | 1.00 | 28.84 | 7 |
| ATOM | 4730 | CA | THR | A | 887 | 84.332 | 50.019 | 93.199 | 1.00 | 28.70 | 6 |
| ATOM | 4731 | CB | THR | A | 887 | 85.273 | 51.101 | 93.744 | 1.00 | 30.92 | 6 |
| ATOM | 4732 | OG1 | THR | A | 887 | 85.105 | 52.305 | 92.975 | 1.00 | 33.55 | 8 |
| ATOM | 4733 | CG2 | THR | A | 887 | 86.709 | 50.644 | 93.680 | 1.00 | 33.87 | 6 |
| ATOM | 4734 | C | THR | A | 887 | 84.663 | 48.664 | 93.827 | 1.00 | 29.33 | 6 |
| ATOM | 4735 | O | THR | A | 887 | 84.160 | 48.324 | 94.908 | 1.00 | 24.17 | 8 |
| ATOM | 4736 | N | ILE | A | 888 | 85.486 | 47.882 | 93.136 | 1.00 | 28.56 | 7 |
| ATOM | 4737 | CA | ILE | A | 888 | 85.893 | 46.577 | 93.648 | 1.00 | 29.62 | 6 |
| ATOM | 4738 | CB | ILE | A | 888 | 86.807 | 45.838 | 92.630 | 1.00 | 30.35 | 6 |
| ATOM | 4739 | CG2 | ILE | A | 888 | 87.544 | 44.690 | 93.309 | 1.00 | 31.74 | 6 |
| ATOM | 4740 | CG1 | ILE | A | 888 | 85.964 | 45.253 | 91.494 | 1.00 | 31.68 | 6 |
| ATOM | 4741 | CD1 | ILE | A | 888 | 85.340 | 46.272 | 90.583 | 1.00 | 37.93 | 6 |
| ATOM | 4742 | C | ILE | A | 888 | 86.627 | 46.771 | 94.984 | 1.00 | 30.72 | 6 |
| ATOM | 4743 | O | ILE | A | 888 | 86.579 | 45.908 | 95.867 | 1.00 | 29.52 | 8 |
| ATOM | 4744 | N | ALA | A | 889 | 87.297 | 47.910 | 95.134 | 1.00 | 29.97 | 7 |
| ATOM | 4745 | CA | ALA | A | 889 | 88.009 | 48.212 | 96.375 | 1.00 | 30.54 | 6 |
| ATOM | 4746 | CB | ALA | A | 889 | 88.838 | 49.484 | 96.211 | 1.00 | 30.03 | 6 |
| ATOM | 4747 | C | ALA | A | 889 | 87.006 | 48.386 | 97.523 | 1.00 | 31.08 | 6 |
| ATOM | 4748 | O | ALA | A | 889 | 87.197 | 47.840 | 98.615 | 1.00 | 29.12 | 8 |
| ATOM | 4749 | N | LYS | A | 890 | 85.937 | 49.148 | 97.284 | 1.00 | 31.92 | 7 |
| ATOM | 4750 | CA | LYS | A | 890 | 84.926 | 49.392 | 98.302 | 1.00 | 35.22 | 6 |
| ATOM | 4751 | CB | LYS | A | 890 | 83.903 | 50.415 | 97.807 | 1.00 | 36.48 | 6 |
| ATOM | 4752 | CG | LYS | A | 890 | 84.497 | 51.771 | 97.464 | 1.00 | 41.75 | 6 |
| ATOM | 4753 | CD | LYS | A | 890 | 84.934 | 52.514 | 98.715 | 1.00 | 46.13 | 6 |
| ATOM | 4754 | CE | LYS | A | 890 | 85.528 | 53.870 | 98.371 | 1.00 | 48.20 | 6 |
| ATOM | 4755 | NZ | LYS | A | 890 | 85.954 | 54.615 | 99.588 | 1.00 | 50.96 | 7 |
| ATOM | 4756 | C | LYS | A | 890 | 84.233 | 48.098 | 98.708 | 1.00 | 36.42 | 6 |
| ATOM | 4757 | O | LYS | A | 890 | 83.821 | 47.938 | 99.868 | 1.00 | 35.79 | 8 |
| ATOM | 4758 | N | ILE | A | 891 | 84.075 | 47.163 | 97.780 | 1.00 | 36.53 | 7 |
| ATOM | 4759 | CA | ILE | A | 891 | 83.438 | 45.893 | 98.098 | 1.00 | 37.57 | 6 |
| ATOM | 4760 | CB | ILE | A | 891 | 83.167 | 45.066 | 96.810 | 1.00 | 36.85 | 6 |
| ATOM | 4761 | CG2 | ILE | A | 891 | 82.697 | 43.667 | 97.167 | 1.00 | 35.40 | 6 |
| ATOM | 4762 | CG1 | ILE | A | 891 | 82.139 | 45.791 | 95.929 | 1.00 | 34.50 | 6 |
| ATOM | 4763 | CD1 | ILE | A | 891 | 81.674 | 44.981 | 94.734 | 1.00 | 36.61 | 6 |
| ATOM | 4764 | C | ILE | A | 891 | 84.350 | 45.199 | 99.065 | 1.00 | 39.09 | 6 |
| ATOM | 4765 | O | ILE | A | 891 | 83.915 | 44.534 | 99.993 | 1.00 | 39.84 | 8 |
| ATOM | 4766 | N | GLN | A | 892 | 85.652 | 45.365 | 98.825 | 1.00 | 39.79 | 7 |
| ATOM | 4767 | CA | GLN | A | 892 | 86.633 | 44.770 | 99.719 | 1.00 | 42.00 | 6 |
| ATOM | 4768 | CB | GLN | A | 892 | 88.063 | 44.827 | 99.118 | 1.00 | 40.36 | 6 |
| ATOM | 4769 | CG | GLN | A | 892 | 88.391 | 43.737 | 98.095 | 1.00 | 40.46 | 6 |
| ATOM | 4770 | CD | GLN | A | 892 | 88.998 | 44.341 | 96.834 | 1.00 | 40.74 | 6 |
| ATOM | 4771 | OE1 | GLN | A | 892 | 88.546 | 45.381 | 96.370 | 1.00 | 41.44 | 8 |
| ATOM | 4772 | NE2 | GLN | A | 892 | 90.017 | 43.854 | 96.122 | 1.00 | 38.32 | 7 |
| ATOM | 4773 | C | GLN | A | 892 | 86.536 | 45.424 | 101.088 | 1.00 | 43.70 | 6 |
| ATOM | 4774 | O | GLN | A | 892 | 86.600 | 44.742 | 102.118 | 1.00 | 43.72 | 8 |
| ATOM | 4775 | N | GLN | A | 893 | 86.371 | 46.719 | 101.117 | 1.00 | 45.30 | 7 |
| ATOM | 4776 | CA | GLN | A | 893 | 86.180 | 47.394 | 102.361 | 1.00 | 48.89 | 6 |
| ATOM | 4777 | CB | GLN | A | 893 | 85.606 | 48.774 | 102.132 | 1.00 | 50.56 | 6 |
| ATOM | 4778 | CG | GLN | A | 893 | 86.603 | 49.869 | 102.230 | 1.00 | 55.16 | 6 |
| ATOM | 4779 | CD | GLN | A | 893 | 85.908 | 51.129 | 102.634 | 1.00 | 56.62 | 6 |
| ATOM | 4780 | OE1 | GLN | A | 893 | 84.811 | 51.427 | 102.159 | 1.00 | 59.63 | 8 |
| ATOM | 4781 | NE2 | GLN | A | 893 | 86.347 | 52.043 | 103.506 | 1.00 | 57.15 | 7 |
| ATOM | 4782 | C | GLN | A | 893 | 85.116 | 46.658 | 103.137 | 1.00 | 49.71 | 6 |
| ATOM | 4783 | O | GLN | A | 893 | 85.397 | 45.885 | 104.058 | 1.00 | 49.41 | 8 |
| ATOM | 4784 | N | SER | A | 894 | 83.865 | 46.931 | 102.722 | 1.00 | 51.67 | 7 |
| ATOM | 4785 | CA | SER | A | 894 | 82.659 | 46.377 | 103.320 | 1.00 | 54.29 | 6 |
| ATOM | 4786 | CB | SER | A | 894 | 81.597 | 46.181 | 102.250 | 1.00 | 54.36 | 6 |
| ATOM | 4787 | OG | SER | A | 894 | 81.271 | 47.414 | 101.633 | 1.00 | 56.48 | 8 |
| ATOM | 4788 | C | SER | A | 894 | 82.998 | 45.103 | 104.135 | 1.00 | 55.85 | 6 |
| ATOM | 4789 | O | SER | A | 894 | 83.117 | 45.213 | 105.358 | 1.00 | 54.78 | 8 |
| ATOM | 4790 | N | THR | A | 895 | 83.155 | 43.921 | 103.546 | 1.00 | 57.42 | 7 |
| ATOM | 4791 | CA | THR | A | 895 | 83.568 | 42.744 | 104.315 | 1.00 | 59.74 | 6 |
| ATOM | 4792 | CB | THR | A | 895 | 84.113 | 41.695 | 103.343 | 1.00 | 59.87 | 6 |
| ATOM | 4793 | OG1 | THR | A | 895 | 83.144 | 41.443 | 102.319 | 1.00 | 61.40 | 8 |
| ATOM | 4794 | CG2 | THR | A | 895 | 84.439 | 40.413 | 104.103 | 1.00 | 61.24 | 6 |
| ATOM | 4795 | C | THR | A | 895 | 84.634 | 43.121 | 105.354 | 1.00 | 61.01 | 6 |
| ATOM | 4796 | O | THR | A | 895 | 84.577 | 42.698 | 106.508 | 1.00 | 60.66 | 8 |
| ATOM | 4797 | N | PHE | A | 902 | 88.425 | 41.072 | 104.267 | 1.00 | 44.77 | 7 |
| ATOM | 4798 | CA | PHE | A | 902 | 88.334 | 39.748 | 104.863 | 1.00 | 44.55 | 6 |
| ATOM | 4799 | CB | PHE | A | 902 | 87.033 | 39.678 | 105.735 | 1.00 | 45.66 | 6 |
| ATOM | 4800 | CG | PHE | A | 902 | 87.112 | 39.536 | 107.278 | 1.00 | 46.64 | 6 |
| ATOM | 4801 | CD1 | PHE | A | 902 | 86.478 | 40.439 | 108.142 | 1.00 | 45.62 | 6 |
| ATOM | 4802 | CD2 | PHE | A | 902 | 87.781 | 38.469 | 107.840 | 1.00 | 46.88 | 6 |
| ATOM | 4803 | CE1 | PHE | A | 902 | 86.511 | 40.253 | 109.514 | 1.00 | 47.22 | 6 |
| ATOM | 4804 | CE2 | PHE | A | 902 | 87.789 | 38.275 | 109.209 | 1.00 | 47.50 | 6 |
| ATOM | 4805 | CZ | PHE | A | 902 | 87.152 | 39.155 | 110.062 | 1.00 | 46.65 | 6 |
| ATOM | 4806 | C | PHE | A | 902 | 88.542 | 38.658 | 103.754 | 1.00 | 43.94 | 6 |
| ATOM | 4807 | O | PHE | A | 902 | 89.436 | 37.830 | 103.980 | 1.00 | 44.97 | 8 |
| ATOM | 4808 | N | LYS | A | 903 | 87.831 | 38.567 | 102.581 | 1.00 | 44.56 | 7 |
| ATOM | 4809 | CA | LYS | A | 903 | 88.264 | 37.491 | 101.650 | 1.00 | 44.97 | 6 |
| ATOM | 4810 | CB | LYS | A | 903 | 87.791 | 36.105 | 102.172 | 1.00 | 46.98 | 6 |
| ATOM | 4811 | CG | LYS | A | 903 | 86.839 | 36.095 | 103.344 | 1.00 | 49.93 | 6 |
| ATOM | 4812 | CD | LYS | A | 903 | 85.905 | 34.900 | 103.278 | 1.00 | 53.08 | 6 |
| ATOM | 4813 | CE | LYS | A | 903 | 86.138 | 33.919 | 104.415 | 1.00 | 54.60 | 6 |
| ATOM | 4814 | NZ | LYS | A | 903 | 85.499 | 32.603 | 104.162 | 1.00 | 56.34 | 7 |
| ATOM | 4815 | C | LYS | A | 903 | 87.918 | 37.545 | 100.136 | 1.00 | 44.75 | 6 |
| ATOM | 4816 | O | LYS | A | 903 | 87.235 | 38.434 | 99.607 | 1.00 | 43.97 | 8 |
| ATOM | 4817 | N | ASP | A | 904 | 88.469 | 36.465 | 99.481 | 1.00 | 43.31 | 7 |
| ATOM | 4818 | CA | ASP | A | 904 | 88.478 | 35.994 | 98.049 | 1.00 | 43.42 | 6 |
| ATOM | 4819 | CB | ASP | A | 904 | 89.423 | 34.747 | 97.912 | 1.00 | 43.24 | 6 |
| ATOM | 4820 | CG | ASP | A | 904 | 90.910 | 34.977 | 97.619 | 1.00 | 43.13 | 6 |
| ATOM | 4821 | OD1 | ASP | A | 904 | 91.625 | 33.993 | 97.353 | 1.00 | 42.69 | 8 |
| ATOM | 4822 | OD2 | ASP | A | 904 | 91.338 | 36.140 | 97.671 | 1.00 | 44.09 | 8 |
| ATOM | 4823 | C | ASP | A | 904 | 87.096 | 35.620 | 97.516 | 1.00 | 42.95 | 6 |
| ATOM | 4824 | O | ASP | A | 904 | 86.927 | 35.337 | 96.331 | 1.00 | 42.01 | 8 |
| ATOM | 4825 | N | GLU | A | 905 | 86.109 | 35.642 | 98.428 | 1.00 | 41.53 | 7 |
| ATOM | 4826 | CA | GLU | A | 905 | 84.691 | 35.323 | 98.206 | 1.00 | 41.31 | 6 |
| ATOM | 4827 | CB | GLU | A | 905 | 84.158 | 34.364 | 99.277 | 1.00 | 44.25 | 6 |
| ATOM | 4828 | CG | GLU | A | 905 | 84.859 | 33.015 | 99.349 | 1.00 | 49.36 | 6 |
| ATOM | 4829 | CD | GLU | A | 905 | 84.304 | 32.136 | 100.463 | 1.00 | 53.55 | 6 |
| ATOM | 4830 | OE1 | GLU | A | 905 | 84.437 | 32.513 | 101.650 | 1.00 | 55.59 | 8 |
| ATOM | 4831 | OE2 | GLU | A | 905 | 83.731 | 31.070 | 100.154 | 1.00 | 56.26 | 8 |
| ATOM | 4832 | C | GLU | A | 905 | 83.838 | 36.586 | 98.236 | 1.00 | 38.94 | 6 |
| ATOM | 4833 | O | GLU | A | 905 | 82.670 | 36.556 | 97.853 | 1.00 | 37.89 | 8 |
| ATOM | 4834 | N | VAL | A | 906 | 84.416 | 37.686 | 98.697 | 1.00 | 38.17 | 7 |
| ATOM | 4835 | CA | VAL | A | 906 | 83.700 | 38.955 | 98.815 | 1.00 | 37.24 | 6 |
| ATOM | 4836 | CB | VAL | A | 906 | 84.680 | 40.100 | 99.160 | 1.00 | 40.88 | 6 |
| ATOM | 4837 | CG1 | VAL | A | 906 | 83.901 | 41.361 | 99.536 | 1.00 | 43.77 | 6 |
| ATOM | 4838 | CG2 | VAL | A | 906 | 85.601 | 39.673 | 100.296 | 1.00 | 41.91 | 6 |
| ATOM | 4839 | C | VAL | A | 906 | 82.877 | 39.395 | 97.604 | 1.00 | 35.69 | 6 |
| ATOM | 4840 | O | VAL | A | 906 | 81.726 | 39.799 | 97.746 | 1.00 | 33.85 | 8 |
| ATOM | 4841 | N | LEU | A | 907 | 83.463 | 39.321 | 96.415 | 1.00 | 36.15 | 7 |
| ATOM | 4842 | CA | LEU | A | 907 | 82.762 | 39.757 | 95.207 | 1.00 | 35.22 | 6 |
| ATOM | 4843 | CB | LEU | A | 907 | 83.700 | 39.720 | 93.996 | 1.00 | 35.57 | 6 |
| ATOM | 4844 | CG | LEU | A | 907 | 83.596 | 40.895 | 93.012 | 1.00 | 37.66 | 6 |
| ATOM | 4845 | CD1 | LEU | A | 907 | 84.311 | 40.517 | 91.726 | 1.00 | 37.57 | 6 |
| ATOM | 4846 | CD2 | LEU | A | 907 | 82.143 | 41.248 | 92.727 | 1.00 | 35.68 | 6 |
| ATOM | 4847 | C | LEU | A | 907 | 81.544 | 38.897 | 94.931 | 1.00 | 33.81 | 6 |
| ATOM | 4848 | O | LEU | A | 907 | 80.441 | 39.410 | 94.686 | 1.00 | 32.25 | 8 |
| ATOM | 4849 | N | ASN | A | 908 | 81.737 | 37.585 | 94.973 | 1.00 | 33.23 | 7 |
| ATOM | 4850 | CA | ASN | A | 908 | 80.630 | 36.671 | 94.726 | 1.00 | 33.17 | 6 |
| ATOM | 4851 | CB | ASN | A | 908 | 81.121 | 35.222 | 94.773 | 1.00 | 34.36 | 6 |
| ATOM | 4852 | CG | ASN | A | 908 | 80.099 | 34.239 | 94.217 | 1.00 | 36.86 | 6 |
| ATOM | 4853 | OD1 | ASN | A | 908 | 79.532 | 33.428 | 94.954 | 1.00 | 34.79 | 8 |
| ATOM | 4854 | ND2 | ASN | A | 908 | 79.852 | 34.318 | 92.909 | 1.00 | 33.09 | 7 |
| ATOM | 4855 | C | ASN | A | 908 | 79.520 | 36.907 | 95.751 | 1.00 | 33.36 | 6 |
| ATOM | 4856 | O | ASN | A | 908 | 78.340 | 36.931 | 95.398 | 1.00 | 31.52 | 8 |
| ATOM | 4857 | N | HIS | A | 909 | 79.905 | 37.081 | 97.023 | 1.00 | 34.07 | 7 |
| ATOM | 4858 | CA | HIS | A | 909 | 78.916 | 37.367 | 98.048 | 1.00 | 35.84 | 6 |
| ATOM | 4859 | CB | HIS | A | 909 | 79.498 | 37.797 | 99.371 | 1.00 | 41.33 | 6 |
| ATOM | 4860 | CG | HIS | A | 909 | 78.338 | 37.833 | 100.394 | 1.00 | 46.80 | 6 |
| ATOM | 4861 | CD2 | HIS | A | 909 | 77.854 | 36.843 | 101.185 | 1.00 | 48.28 | 6 |
| ATOM | 4862 | ND1 | HIS | A | 909 | 77.564 | 38.944 | 100.644 | 1.00 | 48.43 | 7 |
| ATOM | 4863 | CE1 | HIS | A | 909 | 76.648 | 38.638 | 101.555 | 1.00 | 49.32 | 6 |
| ATOM | 4864 | NE2 | HIS | A | 909 | 76.804 | 37.369 | 101.898 | 1.00 | 49.80 | 7 |
| ATOM | 4865 | C | HIS | A | 909 | 78.097 | 38.601 | 97.763 | 1.00 | 35.30 | 6 |
| ATOM | 4866 | O | HIS | A | 909 | 76.872 | 38.606 | 97.795 | 1.00 | 34.56 | 8 |
| ATOM | 4867 | N | TRP | A | 910 | 78.818 | 39.675 | 97.408 | 1.00 | 34.97 | 7 |
| ATOM | 4868 | CA | TRP | A | 910 | 78.205 | 40.955 | 97.071 | 1.00 | 32.70 | 6 |
| ATOM | 4869 | CB | TRP | A | 910 | 79.297 | 41.952 | 96.682 | 1.00 | 33.23 | 6 |
| ATOM | 4870 | CG | TRP | A | 910 | 78.802 | 43.328 | 96.494 | 1.00 | 33.00 | 6 |
| ATOM | 4871 | CD2 | TRP | A | 910 | 78.419 | 43.936 | 95.259 | 1.00 | 34.62 | 6 |
| ATOM | 4872 | CE2 | TRP | A | 910 | 78.023 | 45.261 | 95.551 | 1.00 | 35.38 | 6 |
| ATOM | 4873 | CE3 | TRP | A | 910 | 78.373 | 43.492 | 93.929 | 1.00 | 33.89 | 6 |
| ATOM | 4874 | CD1 | TRP | A | 910 | 78.624 | 44.275 | 97.463 | 1.00 | 34.79 | 6 |
| ATOM | 4875 | NE1 | TRP | A | 910 | 78.158 | 45.440 | 96.904 | 1.00 | 35.64 | 7 |
| ATOM | 4876 | CZ2 | TRP | A | 910 | 77.585 | 46.147 | 94.562 | 1.00 | 35.81 | 6 |
| ATOM | 4877 | CZ3 | TRP | A | 910 | 77.940 | 44.366 | 92.948 | 1.00 | 33.51 | 6 |
| ATOM | 4878 | CH2 | TRP | A | 910 | 77.551 | 45.683 | 93.269 | 1.00 | 37.11 | 6 |
| ATOM | 4879 | C | TRP | A | 910 | 77.189 | 40.835 | 95.930 | 1.00 | 31.61 | 6 |
| ATOM | 4880 | O | TRP | A | 910 | 76.049 | 41.282 | 96.055 | 1.00 | 32.08 | 8 |
| ATOM | 4881 | N | LEU | A | 911 | 77.597 | 40.241 | 94.814 | 1.00 | 31.87 | 7 |
| ATOM | 4882 | CA | LEU | A | 911 | 76.680 | 40.086 | 93.681 | 1.00 | 34.08 | 6 |
| ATOM | 4883 | CB | LEU | A | 911 | 77.386 | 39.433 | 92.490 | 1.00 | 34.75 | 6 |
| ATOM | 4884 | CG | LEU | A | 911 | 78.126 | 40.369 | 91.524 | 1.00 | 38.89 | 6 |
| ATOM | 4885 | CD1 | LEU | A | 911 | 78.802 | 39.536 | 90.431 | 1.00 | 37.09 | 6 |
| ATOM | 4886 | CD2 | LEU | A | 911 | 77.139 | 41.372 | 90.905 | 1.00 | 37.39 | 6 |
| ATOM | 4887 | C | LEU | A | 911 | 75.436 | 39.279 | 94.039 | 1.00 | 33.11 | 6 |
| ATOM | 4888 | O | LEU | A | 911 | 74.325 | 39.607 | 93.606 | 1.00 | 34.10 | 8 |
| ATOM | 4889 | N | LYS | A | 912 | 75.615 | 38.220 | 94.820 | 1.00 | 32.64 | 7 |
| ATOM | 4890 | CA | LYS | A | 912 | 74.482 | 37.402 | 95.220 | 1.00 | 35.60 | 6 |
| ATOM | 4891 | CB | LYS | A | 912 | 74.955 | 36.143 | 95.955 | 1.00 | 38.04 | 6 |
| ATOM | 4892 | CG | LYS | A | 912 | 73.812 | 35.246 | 96.448 | 1.00 | 44.73 | 6 |
| ATOM | 4893 | CD | LYS | A | 912 | 72.804 | 34.944 | 95.339 | 1.00 | 47.24 | 6 |
| ATOM | 4894 | CE | LYS | A | 912 | 71.624 | 34.137 | 95.850 | 1.00 | 50.86 | 6 |
| ATOM | 4895 | NZ | LYS | A | 912 | 70.649 | 33.818 | 94.770 | 1.00 | 51.37 | 7 |
| ATOM | 4896 | C | LYS | A | 912 | 73.528 | 38.194 | 96.108 | 1.00 | 36.43 | 6 |
| ATOM | 4897 | O | LYS | A | 912 | 72.310 | 38.066 | 95.990 | 1.00 | 36.69 | 8 |
| ATOM | 4898 | N | GLU | A | 913 | 74.070 | 39.030 | 96.989 | 1.00 | 37.80 | 7 |
| ATOM | 4899 | CA | GLU | A | 913 | 73.204 | 39.813 | 97.862 | 1.00 | 39.75 | 6 |
| ATOM | 4900 | CB | GLU | A | 913 | 74.005 | 40.405 | 99.022 | 1.00 | 44.08 | 6 |
| ATOM | 4901 | CG | GLU | A | 913 | 73.149 | 41.045 | 100.105 | 1.00 | 50.66 | 6 |
| ATOM | 4902 | CD | GLU | A | 913 | 72.366 | 40.024 | 100.907 | 1.00 | 53.61 | 6 |
| ATOM | 4903 | OE1 | GLU | A | 913 | 72.503 | 38.815 | 100.626 | 1.00 | 56.35 | 8 |
| ATOM | 4904 | OE2 | GLU | A | 913 | 71.613 | 40.434 | 101.816 | 1.00 | 55.71 | 8 |
| ATOM | 4905 | C | GLU | A | 913 | 72.494 | 40.917 | 97.086 | 1.00 | 39.33 | 6 |
| ATOM | 4906 | O | GLU | A | 913 | 71.367 | 41.287 | 97.427 | 1.00 | 37.98 | 8 |
| ATOM | 4907 | N | LYS | A | 914 | 73.125 | 41.459 | 96.030 | 1.00 | 38.45 | 7 |
| ATOM | 4908 | CA | LYS | A | 914 | 72.524 | 42.536 | 95.239 | 1.00 | 38.52 | 6 |
| ATOM | 4909 | CB | LYS | A | 914 | 73.612 | 43.302 | 94.518 | 1.00 | 38.73 | 6 |
| ATOM | 4910 | CG | LYS | A | 914 | 74.338 | 44.302 | 95.400 | 1.00 | 39.67 | 6 |
| ATOM | 4911 | CD | LYS | A | 914 | 73.399 | 45.385 | 95.900 | 1.00 | 41.54 | 6 |
| ATOM | 4912 | CE | LYS | A | 914 | 74.138 | 46.382 | 96.782 | 1.00 | 44.62 | 6 |
| ATOM | 4913 | NZ | LYS | A | 914 | 73.264 | 47.516 | 97.196 | 1.00 | 45.99 | 7 |
| ATOM | 4914 | C | LYS | A | 914 | 71.475 | 42.068 | 94.249 | 1.00 | 39.89 | 6 |
| ATOM | 4915 | O | LYS | A | 914 | 70.618 | 42.853 | 93.828 | 1.00 | 40.63 | 8 |
| ATOM | 4916 | N | SER | A | 915 | 71.498 | 40.800 | 93.874 | 1.00 | 39.59 | 7 |
| ATOM | 4917 | CA | SER | A | 915 | 70.497 | 40.282 | 92.954 | 1.00 | 40.88 | 6 |
| ATOM | 4918 | CB | SER | A | 915 | 71.159 | 39.316 | 91.987 | 1.00 | 41.88 | 6 |
| ATOM | 4919 | OG | SER | A | 915 | 71.605 | 38.155 | 92.665 | 1.00 | 45.55 | 8 |
| ATOM | 4920 | C | SER | A | 915 | 69.374 | 39.519 | 93.655 | 1.00 | 41.11 | 6 |
| ATOM | 4921 | O | SER | A | 915 | 69.618 | 38.709 | 94.547 | 1.00 | 41.13 | 8 |
| ATOM | 4922 | N | PRO | A | 916 | 68.136 | 39.809 | 93.222 | 1.00 | 41.73 | 7 |
| ATOM | 4923 | CD | PRO | A | 916 | 67.687 | 41.222 | 93.300 | 1.00 | 42.00 | 6 |
| ATOM | 4924 | CA | PRO | A | 916 | 66.984 | 39.051 | 93.749 | 1.00 | 41.34 | 6 |
| ATOM | 4925 | CB | PRO | A | 916 | 65.795 | 39.751 | 93.164 | 1.00 | 42.05 | 6 |
| ATOM | 4926 | CG | PRO | A | 916 | 66.198 | 41.162 | 93.242 | 1.00 | 41.79 | 6 |
| ATOM | 4927 | C | PRO | A | 916 | 67.003 | 37.570 | 93.498 | 1.00 | 42.44 | 6 |
| ATOM | 4928 | O | PRO | A | 916 | 67.617 | 36.755 | 94.172 | 1.00 | 42.11 | 8 |
| ATOM | 4929 | N | THR | A | 917 | 66.253 | 37.323 | 92.441 | 1.00 | 41.69 | 7 |
| ATOM | 4930 | CA | THR | A | 917 | 66.005 | 36.060 | 91.833 | 1.00 | 40.36 | 6 |
| ATOM | 4931 | CB | THR | A | 917 | 65.071 | 36.248 | 90.630 | 1.00 | 40.33 | 6 |
| ATOM | 4932 | OG1 | THR | A | 917 | 64.656 | 34.975 | 90.123 | 1.00 | 36.52 | 8 |
| ATOM | 4933 | CG2 | THR | A | 917 | 65.771 | 37.039 | 89.548 | 1.00 | 40.53 | 6 |
| ATOM | 4934 | C | THR | A | 917 | 67.279 | 35.491 | 91.385 | 1.00 | 41.31 | 6 |
| ATOM | 4935 | O | THR | A | 917 | 68.052 | 36.133 | 90.684 | 1.00 | 40.05 | 8 |
| ATOM | 4936 | N | GLU | A | 918 | 67.505 | 34.302 | 91.761 | 1.00 | 41.19 | 7 |
| ATOM | 4937 | CA | GLU | A | 918 | 68.664 | 33.720 | 91.272 | 1.00 | 43.74 | 6 |
| ATOM | 4938 | CB | GLU | A | 918 | 68.674 | 32.250 | 91.618 | 1.00 | 47.26 | 6 |
| ATOM | 4939 | CG | GLU | A | 918 | 69.700 | 31.900 | 92.648 | 1.00 | 51.95 | 6 |
| ATOM | 4940 | CD | GLU | A | 918 | 71.096 | 32.096 | 92.090 | 1.00 | 54.03 | 6 |
| ATOM | 4941 | OE1 | GLU | A | 918 | 71.392 | 31.538 | 91.012 | 1.00 | 56.27 | 8 |
| ATOM | 4942 | OE2 | GLU | A | 918 | 71.902 | 32.803 | 92.729 | 1.00 | 54.71 | 8 |
| ATOM | 4943 | C | GLU | A | 918 | 68.736 | 33.934 | 89.786 | 1.00 | 42.74 | 6 |
| ATOM | 4944 | O | GLU | A | 918 | 69.809 | 34.195 | 89.262 | 1.00 | 43.18 | 8 |
| ATOM | 4945 | N | GLU | A | 919 | 67.604 | 33.840 | 89.084 | 1.00 | 42.58 | 7 |
| ATOM | 4946 | CA | GLU | A | 919 | 67.677 | 34.060 | 87.644 | 1.00 | 42.91 | 6 |
| ATOM | 4947 | CB | GLU | A | 919 | 66.297 | 34.244 | 87.004 | 1.00 | 45.76 | 6 |
| ATOM | 4948 | CG | GLU | A | 919 | 65.548 | 35.497 | 87.403 | 1.00 | 53.56 | 6 |
| ATOM | 4949 | CD | GLU | A | 919 | 64.294 | 35.757 | 86.575 | 1.00 | 57.82 | 6 |
| ATOM | 4950 | OE1 | GLU | A | 919 | 64.427 | 36.020 | 85.355 | 1.00 | 59.06 | 8 |
| ATOM | 4951 | OE2 | GLU | A | 919 | 63.178 | 35.698 | 87.146 | 1.00 | 58.73 | 8 |
| ATOM | 4952 | C | GLU | A | 919 | 68.598 | 35.252 | 87.403 | 1.00 | 40.15 | 6 |
| ATOM | 4953 | O | GLU | A | 919 | 69.588 | 35.117 | 86.675 | 1.00 | 38.49 | 8 |
| ATOM | 4954 | N | LYS | A | 920 | 68.283 | 36.438 | 87.996 | 1.00 | 37.55 | 7 |
| ATOM | 4955 | CA | LYS | A | 920 | 69.083 | 37.659 | 87.763 | 1.00 | 37.31 | 6 |
| ATOM | 4956 | CB | LYS | A | 920 | 68.439 | 38.873 | 88.406 | 1.00 | 36.74 | 6 |
| ATOM | 4957 | CG | LYS | A | 920 | 67.011 | 39.107 | 87.971 | 1.00 | 40.50 | 6 |
| ATOM | 4958 | CD | LYS | A | 920 | 66.540 | 40.502 | 88.363 | 1.00 | 42.38 | 6 |
| ATOM | 4959 | CE | LYS | A | 920 | 65.326 | 40.937 | 87.554 | 1.00 | 44.62 | 6 |
| ATOM | 4960 | NZ | LYS | A | 920 | 64.583 | 42.038 | 88.226 | 1.00 | 48.16 | 7 |
| ATOM | 4961 | C | LYS | A | 920 | 70.510 | 37.550 | 88.264 | 1.00 | 35.44 | 6 |
| ATOM | 4962 | O | LYS | A | 920 | 71.329 | 38.446 | 88.072 | 1.00 | 36.26 | 8 |
| ATOM | 4963 | N | PHE | A | 921 | 70.834 | 36.426 | 88.924 | 1.00 | 32.20 | 7 |
| ATOM | 4964 | CA | PHE | A | 921 | 72.184 | 36.244 | 89.412 | 1.00 | 32.75 | 6 |
| ATOM | 4965 | CB | PHE | A | 921 | 72.218 | 35.570 | 90.765 | 1.00 | 30.95 | 6 |
| ATOM | 4966 | CG | PHE | A | 921 | 73.593 | 35.151 | 91.109 | 1.00 | 30.75 | 6 |
| ATOM | 4967 | CD1 | PHE | A | 921 | 74.599 | 36.097 | 91.285 | 1.00 | 33.00 | 6 |
| ATOM | 4968 | CD2 | PHE | A | 921 | 73.899 | 33.810 | 91.281 | 1.00 | 31.91 | 6 |
| ATOM | 4969 | CE1 | PHE | A | 921 | 75.892 | 35.713 | 91.627 | 1.00 | 32.56 | 6 |
| ATOM | 4970 | CE2 | PHE | A | 921 | 75.190 | 33.416 | 91.654 | 1.00 | 31.39 | 6 |
| ATOM | 4971 | CZ | PHE | A | 921 | 76.187 | 34.372 | 91.825 | 1.00 | 31.78 | 6 |
| ATOM | 4972 | C | PHE | A | 921 | 73.020 | 35.464 | 88.400 | 1.00 | 33.97 | 6 |
| ATOM | 4973 | O | PHE | A | 921 | 74.026 | 35.978 | 87.929 | 1.00 | 34.50 | 8 |
| ATOM | 4974 | N | GLN | A | 922 | 72.624 | 34.245 | 88.071 | 1.00 | 32.35 | 7 |
| ATOM | 4975 | CA | GLN | A | 922 | 73.381 | 33.522 | 87.062 | 1.00 | 32.77 | 6 |
| ATOM | 4976 | CB | GLN | A | 922 | 72.692 | 32.223 | 86.617 | 1.00 | 32.57 | 6 |
| ATOM | 4977 | CG | GLN | A | 922 | 73.070 | 30.933 | 87.377 | 1.00 | 33.59 | 6 |
| ATOM | 4978 | CD | GLN | A | 922 | 74.525 | 30.811 | 87.789 | 1.00 | 33.56 | 6 |
| ATOM | 4979 | OE1 | GLN | A | 922 | 75.436 | 31.061 | 87.001 | 1.00 | 38.03 | 8 |
| ATOM | 4980 | NE2 | GLN | A | 922 | 74.960 | 30.424 | 88.982 | 1.00 | 30.72 | 7 |
| ATOM | 4981 | C | GLN | A | 922 | 73.542 | 34.509 | 85.899 | 1.00 | 31.31 | 6 |
| ATOM | 4982 | O | GLN | A | 922 | 74.576 | 34.512 | 85.245 | 1.00 | 32.17 | 8 |
| ATOM | 4983 | N | ALA | A | 923 | 72.543 | 35.362 | 85.627 | 1.00 | 30.57 | 7 |
| ATOM | 4984 | CA | ALA | A | 923 | 72.632 | 36.352 | 84.507 | 1.00 | 30.26 | 6 |
| ATOM | 4985 | CB | ALA | A | 923 | 71.332 | 37.106 | 84.326 | 1.00 | 30.08 | 6 |
| ATOM | 4986 | C | ALA | A | 923 | 73.778 | 37.341 | 84.779 | 1.00 | 31.19 | 6 |
| ATOM | 4987 | O | ALA | A | 923 | 74.543 | 37.700 | 83.888 | 1.00 | 31.12 | 8 |
| ATOM | 4988 | N | ALA | A | 924 | 73.863 | 37.769 | 86.039 | 1.00 | 29.64 | 7 |
| ATOM | 4989 | CA | ALA | A | 924 | 74.876 | 38.680 | 86.510 | 1.00 | 28.94 | 6 |
| ATOM | 4990 | CB | ALA | A | 924 | 74.591 | 39.105 | 87.933 | 1.00 | 29.70 | 6 |
| ATOM | 4991 | C | ALA | A | 924 | 76.259 | 38.033 | 86.419 | 1.00 | 29.17 | 6 |
| ATOM | 4992 | O | ALA | A | 924 | 77.226 | 38.671 | 85.976 | 1.00 | 28.41 | 8 |
| ATOM | 4993 | N | VAL | A | 925 | 76.358 | 36.773 | 86.856 | 1.00 | 27.66 | 7 |
| ATOM | 4994 | CA | VAL | A | 925 | 77.600 | 36.059 | 86.766 | 1.00 | 27.21 | 6 |
| ATOM | 4995 | CB | VAL | A | 925 | 77.475 | 34.644 | 87.392 | 1.00 | 28.63 | 6 |
| ATOM | 4996 | CG1 | VAL | A | 925 | 78.763 | 33.870 | 87.230 | 1.00 | 28.37 | 6 |
| ATOM | 4997 | CG2 | VAL | A | 925 | 77.074 | 34.743 | 88.856 | 1.00 | 25.58 | 6 |
| ATOM | 4998 | C | VAL | A | 925 | 77.997 | 35.951 | 85.296 | 1.00 | 26.27 | 6 |
| ATOM | 4999 | O | VAL | A | 925 | 79.182 | 36.081 | 84.969 | 1.00 | 25.78 | 8 |
| ATOM | 5000 | N | GLU | A | 926 | 77.051 | 35.707 | 84.371 | 1.00 | 29.62 | 7 |
| ATOM | 5001 | CA | GLU | A | 926 | 77.458 | 35.621 | 82.945 | 1.00 | 30.79 | 6 |
| ATOM | 5002 | CB | GLU | A | 926 | 76.420 | 34.947 | 82.051 | 1.00 | 32.29 | 6 |
| ATOM | 5003 | CG | GLU | A | 926 | 77.076 | 33.752 | 81.363 | 1.00 | 38.41 | 6 |
| ATOM | 5004 | CD | GLU | A | 926 | 76.153 | 32.921 | 80.496 | 1.00 | 44.82 | 6 |
| ATOM | 5005 | OE1 | GLU | A | 926 | 75.191 | 33.497 | 79.915 | 1.00 | 46.32 | 8 |
| ATOM | 5006 | OE2 | GLU | A | 926 | 76.383 | 31.691 | 80.408 | 1.00 | 47.36 | 8 |
| ATOM | 5007 | C | GLU | A | 926 | 77.812 | 36.982 | 82.388 | 1.00 | 30.93 | 6 |
| ATOM | 5008 | O | GLU | A | 926 | 78.601 | 37.088 | 81.449 | 1.00 | 29.08 | 8 |
| ATOM | 5009 | N | ARG | A | 927 | 77.253 | 38.028 | 82.947 | 1.00 | 30.71 | 7 |
| ATOM | 5010 | CA | ARG | A | 927 | 77.623 | 39.331 | 82.464 | 1.00 | 30.76 | 6 |
| ATOM | 5011 | CB | ARG | A | 927 | 76.632 | 40.377 | 82.981 | 1.00 | 33.32 | 6 |
| ATOM | 5012 | CG | ARG | A | 927 | 76.500 | 41.594 | 82.088 | 1.00 | 35.68 | 6 |
| ATOM | 5013 | CD | ARG | A | 927 | 75.393 | 42.496 | 82.583 | 1.00 | 39.83 | 6 |
| ATOM | 5014 | NE | ARG | A | 927 | 74.941 | 43.404 | 81.533 | 1.00 | 45.39 | 7 |
| ATOM | 5015 | CZ | ARG | A | 927 | 74.252 | 43.021 | 80.462 | 1.00 | 46.78 | 6 |
| ATOM | 5016 | NH1 | ARG | A | 927 | 73.922 | 41.742 | 80.297 | 1.00 | 45.40 | 7 |
| ATOM | 5017 | NH2 | ARG | A | 927 | 73.879 | 43.914 | 79.551 | 1.00 | 46.12 | 7 |
| ATOM | 5018 | C | ARG | A | 927 | 79.031 | 39.682 | 82.922 | 1.00 | 28.13 | 6 |
| ATOM | 5019 | O | ARG | A | 927 | 79.757 | 40.405 | 82.235 | 1.00 | 26.33 | 8 |
| ATOM | 5020 | N | PHE | A | 928 | 79.408 | 39.163 | 84.089 | 1.00 | 26.77 | 7 |
| ATOM | 5021 | CA | PHE | A | 928 | 80.732 | 39.389 | 84.665 | 1.00 | 27.71 | 6 |
| ATOM | 5022 | CB | PHE | A | 928 | 80.773 | 38.912 | 86.121 | 1.00 | 28.88 | 6 |
| ATOM | 5023 | CG | PHE | A | 928 | 82.141 | 38.999 | 86.743 | 1.00 | 28.68 | 6 |
| ATOM | 5024 | CD1 | PHE | A | 928 | 82.651 | 40.225 | 87.171 | 1.00 | 28.32 | 6 |
| ATOM | 5025 | CD2 | PHE | A | 928 | 82.935 | 37.864 | 86.871 | 1.00 | 26.65 | 6 |
| ATOM | 5026 | CE1 | PHE | A | 928 | 83.933 | 40.318 | 87.716 | 1.00 | 26.88 | 6 |
| ATOM | 5027 | CE2 | PHE | A | 928 | 84.214 | 37.947 | 87.412 | 1.00 | 27.96 | 6 |
| ATOM | 5028 | CZ | PHE | A | 928 | 84.714 | 39.175 | 87.835 | 1.00 | 27.48 | 6 |
| ATOM | 5029 | C | PHE | A | 928 | 81.811 | 38.639 | 83.890 | 1.00 | 26.97 | 6 |
| ATOM | 5030 | O | PHE | A | 928 | 82.916 | 39.139 | 83.693 | 1.00 | 28.16 | 8 |
| ATOM | 5031 | N | VAL | A | 929 | 81.504 | 37.418 | 83.478 | 1.00 | 26.50 | 7 |
| ATOM | 5032 | CA | VAL | A | 929 | 82.476 | 36.632 | 82.734 | 1.00 | 24.93 | 6 |
| ATOM | 5033 | CB | VAL | A | 929 | 81.915 | 35.221 | 82.413 | 1.00 | 25.58 | 6 |
| ATOM | 5034 | CG1 | VAL | A | 929 | 82.761 | 34.539 | 81.347 | 1.00 | 25.40 | 6 |
| ATOM | 5035 | CG2 | VAL | A | 929 | 81.923 | 34.374 | 83.688 | 1.00 | 25.05 | 6 |
| ATOM | 5036 | C | VAL | A | 929 | 82.829 | 37.378 | 81.455 | 1.00 | 22.94 | 6 |
| ATOM | 5037 | O | VAL | A | 929 | 83.993 | 37.629 | 81.178 | 1.00 | 24.72 | 8 |
| ATOM | 5038 | N | TYR | A | 930 | 81.809 | 37.760 | 80.698 | 1.00 | 23.58 | 7 |
| ATOM | 5039 | CA | TYR | A | 930 | 82.006 | 38.492 | 79.458 | 1.00 | 26.14 | 6 |
| ATOM | 5040 | CB | TYR | A | 930 | 80.668 | 38.713 | 78.762 | 1.00 | 27.98 | 6 |
| ATOM | 5041 | CG | TYR | A | 930 | 80.173 | 37.530 | 77.966 | 1.00 | 36.84 | 6 |
| ATOM | 5042 | CD1 | TYR | A | 930 | 79.746 | 36.362 | 78.600 | 1.00 | 38.14 | 6 |
| ATOM | 5043 | CE1 | TYR | A | 930 | 79.286 | 35.274 | 77.863 | 1.00 | 41.27 | 6 |
| ATOM | 5044 | CD2 | TYR | A | 930 | 80.128 | 37.577 | 76.569 | 1.00 | 38.11 | 6 |
| ATOM | 5045 | CE2 | TYR | A | 930 | 79.671 | 36.493 | 75.824 | 1.00 | 40.99 | 6 |
| ATOM | 5046 | CZ | TYR | A | 930 | 79.252 | 35.347 | 76.477 | 1.00 | 42.07 | 6 |
| ATOM | 5047 | OH | TYR | A | 930 | 78.801 | 34.269 | 75.744 | 1.00 | 45.19 | 8 |
| ATOM | 5048 | C | TYR | A | 930 | 82.695 | 39.850 | 79.657 | 1.00 | 25.01 | 6 |
| ATOM | 5049 | O | TYR | A | 930 | 83.724 | 40.115 | 79.036 | 1.00 | 25.77 | 8 |
| ATOM | 5050 | N | SER | A | 931 | 82.132 | 40.708 | 80.510 | 1.00 | 24.83 | 7 |
| ATOM | 5051 | CA | SER | A | 931 | 82.717 | 42.040 | 80.739 | 1.00 | 22.92 | 6 |
| ATOM | 5052 | CB | SER | A | 931 | 81.852 | 42.870 | 81.714 | 1.00 | 25.22 | 6 |
| ATOM | 5053 | OG | SER | A | 931 | 81.823 | 42.324 | 83.032 | 1.00 | 26.27 | 8 |
| ATOM | 5054 | C | SER | A | 931 | 84.149 | 41.933 | 81.246 | 1.00 | 23.43 | 6 |
| ATOM | 5055 | O | SER | A | 931 | 85.016 | 42.721 | 80.860 | 1.00 | 21.98 | 8 |
| ATOM | 5056 | N | CYS | A | 932 | 84.414 | 40.949 | 82.099 | 1.00 | 23.61 | 7 |
| ATOM | 5057 | CA | CYS | A | 932 | 85.764 | 40.771 | 82.599 | 1.00 | 23.47 | 6 |
| ATOM | 5058 | CB | CYS | A | 932 | 85.818 | 39.637 | 83.625 | 1.00 | 25.33 | 6 |
| ATOM | 5059 | SG | CYS | A | 932 | 87.438 | 39.452 | 84.403 | 1.00 | 33.59 | 16 |
| ATOM | 5060 | C | CYS | A | 932 | 86.689 | 40.451 | 81.424 | 1.00 | 24.10 | 6 |
| ATOM | 5061 | O | CYS | A | 932 | 87.764 | 41.030 | 81.294 | 1.00 | 23.84 | 8 |
| ATOM | 5062 | N | ALA | A | 933 | 86.271 | 39.525 | 80.563 | 1.00 | 25.16 | 7 |
| ATOM | 5063 | CA | ALA | A | 933 | 87.078 | 39.157 | 79.396 | 1.00 | 25.29 | 6 |
| ATOM | 5064 | CB | ALA | A | 933 | 86.387 | 38.026 | 78.604 | 1.00 | 23.73 | 6 |
| ATOM | 5065 | C | ALA | A | 933 | 87.270 | 40.388 | 78.500 | 1.00 | 24.29 | 6 |
| ATOM | 5066 | O | ALA | A | 933 | 86.368 | 40.659 | 78.022 | 1.00 | 24.42 | 8 |
| ATOM | 5067 | N | GLY | A | 934 | 86.189 | 41.125 | 78.267 | 1.00 | 24.49 | 7 |
| ATOM | 5068 | CA | GLY | A | 934 | 86.281 | 42.320 | 77.442 | 1.00 | 23.51 | 6 |
| ATOM | 5069 | C | GLY | A | 934 | 87.325 | 43.298 | 77.964 | 1.00 | 23.93 | 6 |
| ATOM | 5070 | O | GLY | A | 934 | 88.160 | 43.782 | 77.205 | 1.00 | 24.08 | 8 |
| ATOM | 5071 | N | TYR | A | 935 | 87.300 | 43.597 | 79.260 | 1.00 | 22.56 | 7 |
| ATOM | 5072 | CA | TYR | A | 935 | 88.288 | 44.534 | 79.785 | 1.00 | 22.62 | 6 |
| ATOM | 5073 | CB | TYR | A | 935 | 87.838 | 45.088 | 81.150 | 1.00 | 21.28 | 6 |
| ATOM | 5074 | CG | TYR | A | 935 | 87.022 | 46.355 | 80.997 | 1.00 | 17.20 | 6 |
| ATOM | 5075 | CD1 | TYR | A | 935 | 85.637 | 46.310 | 80.895 | 1.00 | 16.09 | 6 |
| ATOM | 5076 | CE1 | TYR | A | 935 | 84.889 | 47.470 | 80.631 | 1.00 | 20.12 | 6 |
| ATOM | 5077 | CD2 | TYR | A | 935 | 87.659 | 47.599 | 80.835 | 1.00 | 21.46 | 6 |
| ATOM | 5078 | CE2 | TYR | A | 935 | 86.917 | 48.769 | 80.551 | 1.00 | 18.58 | 6 |
| ATOM | 5079 | CZ | TYR | A | 935 | 85.544 | 48.692 | 80.455 | 1.00 | 16.08 | 6 |
| ATOM | 5080 | OH | TYR | A | 935 | 84.814 | 49.823 | 80.170 | 1.00 | 21.37 | 8 |
| ATOM | 5081 | C | TYR | A | 935 | 89.705 | 43.966 | 79.860 | 1.00 | 21.52 | 6 |
| ATOM | 5082 | O | TYR | A | 935 | 90.671 | 44.695 | 79.674 | 1.00 | 21.37 | 8 |
| ATOM | 5083 | N | CYS | A | 936 | 89.850 | 42.672 | 80.112 | 1.00 | 23.71 | 7 |
| ATOM | 5084 | CA | CYS | A | 936 | 91.197 | 42.107 | 80.173 | 1.00 | 23.92 | 6 |
| ATOM | 5085 | CB | CYS | A | 936 | 91.164 | 40.631 | 80.592 | 1.00 | 28.07 | 6 |
| ATOM | 5086 | SG | CYS | A | 936 | 90.899 | 40.315 | 82.385 | 1.00 | 30.68 | 16 |
| ATOM | 5087 | C | CYS | A | 936 | 91.860 | 42.236 | 78.807 | 1.00 | 25.62 | 6 |
| ATOM | 5088 | O | CYS | A | 936 | 93.036 | 42.570 | 78.707 | 1.00 | 27.39 | 8 |
| ATOM | 5089 | N | VAL | A | 937 | 91.102 | 41.977 | 77.746 | 1.00 | 25.56 | 7 |
| ATOM | 5090 | CA | VAL | A | 937 | 91.657 | 42.079 | 76.400 | 1.00 | 25.96 | 6 |
| ATOM | 5091 | CB | VAL | A | 937 | 90.721 | 41.422 | 75.351 | 1.00 | 25.04 | 6 |
| ATOM | 5092 | CG1 | VAL | A | 937 | 91.234 | 41.692 | 73.948 | 1.00 | 23.98 | 6 |
| ATOM | 5093 | CG2 | VAL | A | 937 | 90.634 | 39.910 | 75.608 | 1.00 | 28.43 | 6 |
| ATOM | 5094 | C | VAL | A | 937 | 91.891 | 43.540 | 76.015 | 1.00 | 22.98 | 6 |
| ATOM | 5095 | O | VAL | A | 937 | 92.974 | 43.894 | 75.581 | 1.00 | 19.75 | 8 |
| ATOM | 5096 | N | ALA | A | 938 | 90.869 | 44.377 | 76.175 | 1.00 | 23.05 | 7 |
| ATOM | 5097 | CA | ALA | A | 938 | 90.987 | 45.788 | 75.836 | 1.00 | 23.27 | 6 |
| ATOM | 5098 | CB | ALA | A | 938 | 89.683 | 46.514 | 76.163 | 1.00 | 21.59 | 6 |
| ATOM | 5099 | C | ALA | A | 938 | 92.153 | 46.478 | 76.537 | 1.00 | 23.00 | 6 |
| ATOM | 5100 | O | ALA | A | 938 | 92.992 | 47.112 | 75.895 | 1.00 | 23.96 | 8 |
| ATOM | 5101 | N | THR | A | 939 | 92.222 | 46.338 | 77.853 | 1.00 | 24.10 | 7 |
| ATOM | 5102 | CA | THR | A | 939 | 93.271 | 46.998 | 78.625 | 1.00 | 24.89 | 6 |
| ATOM | 5103 | CB | THR | A | 939 | 92.972 | 46.915 | 80.135 | 1.00 | 24.82 | 6 |
| ATOM | 5104 | OG1 | THR | A | 939 | 93.882 | 47.754 | 80.838 | 1.00 | 35.85 | 8 |
| ATOM | 5105 | CG2 | THR | A | 939 | 93.147 | 45.525 | 80.636 | 1.00 | 21.76 | 6 |
| ATOM | 5106 | C | THR | A | 939 | 94.676 | 46.468 | 78.342 | 1.00 | 23.64 | 6 |
| ATOM | 5107 | O | THR | A | 939 | 95.647 | 47.219 | 78.397 | 1.00 | 20.70 | 8 |
| ATOM | 5108 | N | PHE | A | 940 | 94.793 | 45.175 | 78.058 | 1.00 | 23.55 | 7 |
| ATOM | 5109 | CA | PHE | A | 940 | 96.104 | 44.602 | 77.731 | 1.00 | 25.49 | 6 |
| ATOM | 5110 | CB | PHE | A | 940 | 95.999 | 43.084 | 77.543 | 1.00 | 26.06 | 6 |
| ATOM | 5111 | CG | PHE | A | 940 | 97.289 | 42.444 | 77.105 | 1.00 | 26.78 | 6 |
| ATOM | 5112 | CD1 | PHE | A | 940 | 98.396 | 42.441 | 77.946 | 1.00 | 27.32 | 6 |
| ATOM | 5113 | CD2 | PHE | A | 940 | 97.407 | 41.881 | 75.835 | 1.00 | 28.12 | 6 |
| ATOM | 5114 | CE1 | PHE | A | 940 | 99.609 | 41.885 | 77.530 | 1.00 | 28.95 | 6 |
| ATOM | 5115 | CE2 | PHE | A | 940 | 98.620 | 41.319 | 75.406 | 1.00 | 29.15 | 6 |
| ATOM | 5116 | CZ | PHE | A | 940 | 99.719 | 41.322 | 76.254 | 1.00 | 28.05 | 6 |
| ATOM | 5117 | C | PHE | A | 940 | 96.620 | 45.230 | 76.426 | 1.00 | 24.52 | 6 |
| ATOM | 5118 | O | PHE | A | 940 | 97.751 | 45.703 | 76.339 | 1.00 | 25.64 | 8 |
| ATOM | 5119 | N | VAL | A | 941 | 95.776 | 45.244 | 75.408 | 1.00 | 27.01 | 7 |
| ATOM | 5120 | CA | VAL | A | 941 | 96.177 | 45.815 | 74.130 | 1.00 | 26.56 | 6 |
| ATOM | 5121 | CB | VAL | A | 941 | 95.038 | 45.670 | 73.111 | 1.00 | 28.51 | 6 |
| ATOM | 5122 | CG1 | VAL | A | 941 | 95.366 | 46.426 | 71.820 | 1.00 | 28.54 | 6 |
| ATOM | 5123 | CG2 | VAL | A | 941 | 94.829 | 44.182 | 72.818 | 1.00 | 31.97 | 6 |
| ATOM | 5124 | C | VAL | A | 941 | 96.616 | 47.279 | 74.259 | 1.00 | 26.89 | 6 |
| ATOM | 5125 | O | VAL | A | 941 | 97.634 | 47.680 | 73.694 | 1.00 | 24.15 | 8 |
| ATOM | 5126 | N | LEU | A | 942 | 95.861 | 48.065 | 75.024 | 1.00 | 27.42 | 7 |
| ATOM | 5127 | CA | LEU | A | 942 | 96.169 | 49.478 | 75.223 | 1.00 | 26.63 | 6 |
| ATOM | 5128 | CB | LEU | A | 942 | 94.883 | 50.243 | 75.568 | 1.00 | 25.46 | 6 |
| ATOM | 5129 | CG | LEU | A | 942 | 93.817 | 50.260 | 74.465 | 1.00 | 27.81 | 6 |
| ATOM | 5130 | CD1 | LEU | A | 942 | 92.543 | 50.887 | 74.971 | 1.00 | 26.84 | 6 |
| ATOM | 5131 | CD2 | LEU | A | 942 | 94.353 | 51.023 | 73.257 | 1.00 | 27.22 | 6 |
| ATOM | 5132 | C | LEU | A | 942 | 97.234 | 49.723 | 76.295 | 1.00 | 27.43 | 6 |
| ATOM | 5133 | O | LEU | A | 942 | 97.536 | 50.866 | 76.626 | 1.00 | 25.94 | 8 |
| ATOM | 5134 | N | GLY | A | 943 | 97.795 | 48.650 | 76.842 | 1.00 | 28.17 | 7 |
| ATOM | 5135 | CA | GLY | A | 943 | 98.831 | 48.789 | 77.850 | 1.00 | 27.03 | 6 |
| ATOM | 5136 | C | GLY | A | 943 | 98.440 | 49.476 | 79.141 | 1.00 | 28.17 | 6 |
| ATOM | 5137 | O | GLY | A | 943 | 99.275 | 50.118 | 79.778 | 1.00 | 29.42 | 8 |
| ATOM | 5138 | N | ILE | A | 944 | 97.180 | 49.354 | 79.539 | 1.00 | 27.49 | 7 |
| ATOM | 5139 | CA | ILE | A | 944 | 96.725 | 49.975 | 80.781 | 1.00 | 28.15 | 6 |
| ATOM | 5140 | CB | ILE | A | 944 | 95.719 | 51.098 | 80.492 | 1.00 | 26.59 | 6 |
| ATOM | 5141 | CG2 | ILE | A | 944 | 96.391 | 52.193 | 79.665 | 1.00 | 27.24 | 6 |
| ATOM | 5142 | CG1 | ILE | A | 944 | 94.512 | 50.519 | 79.746 | 1.00 | 27.72 | 6 |
| ATOM | 5143 | CD1 | ILE | A | 944 | 93.423 | 51.527 | 79.440 | 1.00 | 32.18 | 6 |
| ATOM | 5144 | C | ILE | A | 944 | 96.061 | 48.921 | 81.665 | 1.00 | 28.63 | 6 |
| ATOM | 5145 | O | ILE | A | 944 | 95.136 | 49.221 | 82.430 | 1.00 | 28.50 | 8 |
| ATOM | 5146 | N | GLY | A | 945 | 96.539 | 47.684 | 81.564 | 1.00 | 27.53 | 7 |
| ATOM | 5147 | CA | GLY | A | 945 | 95.949 | 46.610 | 82.345 | 1.00 | 28.27 | 6 |
| ATOM | 5148 | C | GLY | A | 945 | 96.313 | 46.506 | 83.819 | 1.00 | 28.76 | 6 |
| ATOM | 5149 | O | GLY | A | 945 | 95.500 | 46.029 | 84.618 | 1.00 | 27.40 | 8 |
| ATOM | 5150 | N | ASP | A | 946 | 97.514 | 46.946 | 84.187 | 1.00 | 28.53 | 7 |
| ATOM | 5151 | CA | ASP | A | 946 | 97.971 | 46.854 | 85.578 | 1.00 | 29.50 | 6 |
| ATOM | 5152 | CB | ASP | A | 946 | 99.494 | 46.939 | 85.649 | 1.00 | 31.27 | 6 |
| ATOM | 5153 | CG | ASP | A | 946 | 100.041 | 46.486 | 87.005 | 1.00 | 34.81 | 6 |
| ATOM | 5154 | OD1 | ASP | A | 946 | 99.260 | 46.443 | 87.984 | 1.00 | 34.71 | 8 |
| ATOM | 5155 | OD2 | ASP | A | 946 | 101.246 | 46.178 | 87.089 | 1.00 | 35.59 | 8 |
| ATOM | 5156 | C | ASP | A | 946 | 97.363 | 47.960 | 86.422 | 1.00 | 28.21 | 6 |
| ATOM | 5157 | O | ASP | A | 946 | 97.982 | 48.998 | 86.648 | 1.00 | 27.40 | 8 |
| ATOM | 5158 | N | ARG | A | 947 | 96.157 | 47.720 | 86.913 | 1.00 | 27.61 | 7 |
| ATOM | 5159 | CA | ARG | A | 947 | 95.461 | 48.728 | 87.689 | 1.00 | 29.78 | 6 |
| ATOM | 5160 | CB | ARG | A | 947 | 94.144 | 49.068 | 86.997 | 1.00 | 26.98 | 6 |
| ATOM | 5161 | CG | ARG | A | 947 | 94.367 | 49.525 | 85.561 | 1.00 | 28.90 | 6 |
| ATOM | 5162 | CD | ARG | A | 947 | 94.618 | 51.009 | 85.519 | 1.00 | 31.29 | 6 |
| ATOM | 5163 | NE | ARG | A | 947 | 93.460 | 51.673 | 84.941 | 1.00 | 37.62 | 7 |
| ATOM | 5164 | CZ | ARG | A | 947 | 92.921 | 52.789 | 85.400 | 1.00 | 36.41 | 6 |
| ATOM | 5165 | NH1 | ARG | A | 947 | 93.429 | 53.399 | 86.467 | 1.00 | 38.13 | 7 |
| ATOM | 5166 | NH2 | ARG | A | 947 | 91.864 | 53.316 | 84.787 | 1.00 | 38.45 | 7 |
| ATOM | 5167 | C | ARG | A | 947 | 95.177 | 48.354 | 89.130 | 1.00 | 30.77 | 6 |
| ATOM | 5168 | O | ARG | A | 947 | 95.160 | 47.184 | 89.520 | 1.00 | 28.88 | 8 |
| ATOM | 5169 | N | HIS | A | 948 | 94.950 | 49.389 | 89.909 | 1.00 | 32.18 | 7 |
| ATOM | 5170 | CA | HIS | A | 948 | 94.589 | 49.273 | 91.306 | 1.00 | 31.74 | 6 |
| ATOM | 5171 | CB | HIS | A | 948 | 95.075 | 50.495 | 92.036 | 1.00 | 35.02 | 6 |
| ATOM | 5172 | CG | HIS | A | 948 | 94.614 | 50.585 | 93.498 | 1.00 | 36.78 | 6 |
| ATOM | 5173 | CD2 | HIS | A | 948 | 94.158 | 51.643 | 94.212 | 1.00 | 37.70 | 6 |
| ATOM | 5174 | ND1 | HIS | A | 948 | 94.569 | 49.502 | 94.351 | 1.00 | 39.60 | 7 |
| ATOM | 5175 | CE1 | HIS | A | 948 | 94.108 | 49.889 | 95.527 | 1.00 | 39.36 | 6 |
| ATOM | 5176 | NE2 | HIS | A | 948 | 93.851 | 51.184 | 95.469 | 1.00 | 38.54 | 7 |
| ATOM | 5177 | C | HIS | A | 948 | 93.074 | 49.048 | 91.341 | 1.00 | 31.52 | 6 |
| ATOM | 5178 | O | HIS | A | 948 | 92.429 | 49.389 | 90.346 | 1.00 | 27.23 | 8 |
| ATOM | 5179 | N | ASN | A | 949 | 92.452 | 48.419 | 92.367 | 1.00 | 30.16 | 7 |
| ATOM | 5180 | CA | ASN | A | 949 | 90.982 | 48.203 | 92.275 | 1.00 | 33.49 | 6 |
| ATOM | 5181 | CB | ASN | A | 949 | 90.446 | 47.411 | 93.451 | 1.00 | 38.83 | 6 |
| ATOM | 5182 | CG | ASN | A | 949 | 91.528 | 46.611 | 94.111 | 1.00 | 43.48 | 6 |
| ATOM | 5183 | OD1 | ASN | A | 949 | 92.686 | 46.626 | 93.676 | 1.00 | 49.10 | 8 |
| ATOM | 5184 | ND2 | ASN | A | 949 | 91.172 | 45.884 | 95.167 | 1.00 | 41.84 | 7 |
| ATOM | 5185 | C | ASN | A | 949 | 90.380 | 49.565 | 92.219 | 1.00 | 32.97 | 6 |
| ATOM | 5186 | O | ASN | A | 949 | 89.976 | 50.064 | 91.162 | 1.00 | 33.92 | 8 |
| ATOM | 5187 | N | ASP | A | 950 | 90.326 | 50.180 | 93.386 | 1.00 | 31.20 | 7 |
| ATOM | 5188 | CA | ASP | A | 950 | 89.995 | 51.558 | 93.480 | 1.00 | 30.79 | 6 |
| ATOM | 5189 | CB | ASP | A | 950 | 91.310 | 52.295 | 93.797 | 1.00 | 34.28 | 6 |
| ATOM | 5190 | CG | ASP | A | 950 | 91.310 | 53.336 | 94.889 | 1.00 | 36.96 | 6 |
| ATOM | 5191 | OD1 | ASP | A | 950 | 90.764 | 53.053 | 95.972 | 1.00 | 37.18 | 8 |
| ATOM | 5192 | OD2 | ASP | A | 950 | 91.872 | 54.427 | 94.667 | 1.00 | 40.02 | 8 |
| ATOM | 5193 | C | ASP | A | 950 | 89.498 | 52.116 | 92.156 | 1.00 | 31.17 | 6 |
| ATOM | 5194 | O | ASP | A | 950 | 88.575 | 52.915 | 92.095 | 1.00 | 29.80 | 8 |
| ATOM | 5195 | N | ASN | A | 951 | 90.180 | 51.654 | 91.100 | 1.00 | 28.84 | 7 |
| ATOM | 5196 | CA | ASN | A | 951 | 89.994 | 52.083 | 89.699 | 1.00 | 27.59 | 6 |
| ATOM | 5197 | CB | ASN | A | 951 | 91.368 | 52.401 | 89.089 | 1.00 | 28.57 | 6 |
| ATOM | 5198 | CG | ASN | A | 951 | 92.155 | 53.424 | 89.867 | 1.00 | 30.95 | 6 |
| ATOM | 5199 | OD1 | ASN | A | 951 | 93.365 | 53.283 | 90.057 | 1.00 | 29.91 | 8 |
| ATOM | 5200 | ND2 | ASN | A | 951 | 91.472 | 54.462 | 90.333 | 1.00 | 28.90 | 7 |
| ATOM | 5201 | C | ASN | A | 951 | 89.252 | 51.093 | 88.791 | 1.00 | 26.53 | 6 |
| ATOM | 5202 | O | ASN | A | 951 | 89.335 | 51.165 | 87.560 | 1.00 | 25.92 | 8 |
| ATOM | 5203 | N | ILE | A | 952 | 88.546 | 50.155 | 89.411 | 1.00 | 25.51 | 7 |
| ATOM | 5204 | CA | ILE | A | 952 | 87.733 | 49.188 | 88.687 | 1.00 | 23.49 | 6 |
| ATOM | 5205 | CB | ILE | A | 952 | 88.358 | 47.779 | 88.698 | 1.00 | 25.21 | 6 |
| ATOM | 5206 | CG2 | ILE | A | 952 | 87.443 | 46.791 | 87.963 | 1.00 | 22.97 | 6 |
| ATOM | 5207 | CG1 | ILE | A | 952 | 89.736 | 47.818 | 88.042 | 1.00 | 24.01 | 6 |
| ATOM | 5208 | CD1 | ILE | A | 952 | 90.411 | 46.466 | 87.985 | 1.00 | 24.49 | 6 |
| ATOM | 5209 | C | ILE | A | 952 | 86.389 | 49.106 | 89.386 | 1.00 | 24.39 | 6 |
| ATOM | 5210 | O | ILE | A | 952 | 86.326 | 48.954 | 90.611 | 1.00 | 23.34 | 8 |
| ATOM | 5211 | N | MET | A | 953 | 85.315 | 49.229 | 88.616 | 1.00 | 22.84 | 7 |
| ATOM | 5212 | CA | MET | A | 953 | 83.980 | 49.146 | 89.180 | 1.00 | 23.47 | 6 |
| ATOM | 5213 | CB | MET | A | 953 | 83.209 | 50.446 | 88.919 | 1.00 | 24.10 | 6 |
| ATOM | 5214 | CG | MET | A | 953 | 83.851 | 51.672 | 89.567 | 1.00 | 27.68 | 6 |
| ATOM | 5215 | SD | MET | A | 953 | 82.869 | 53.173 | 89.359 | 1.00 | 32.58 | 16 |
| ATOM | 5216 | CE | MET | A | 953 | 81.557 | 52.885 | 90.549 | 1.00 | 30.06 | 6 |
| ATOM | 5217 | C | MET | A | 953 | 83.195 | 47.958 | 88.639 | 1.00 | 24.00 | 6 |
| ATOM | 5218 | O | MET | A | 953 | 83.580 | 47.334 | 87.645 | 1.00 | 25.29 | 8 |
| ATOM | 5219 | N | ILE | A | 954 | 82.090 | 47.658 | 89.309 | 1.00 | 23.16 | 7 |
| ATOM | 5220 | CA | ILE | A | 954 | 81.206 | 46.563 | 88.939 | 1.00 | 23.63 | 6 |
| ATOM | 5221 | CB | ILE | A | 954 | 81.490 | 45.310 | 89.796 | 1.00 | 24.30 | 6 |
| ATOM | 5222 | CG2 | ILE | A | 954 | 81.217 | 45.621 | 91.270 | 1.00 | 22.16 | 6 |
| ATOM | 5223 | CG1 | ILE | A | 954 | 80.586 | 44.146 | 89.374 | 1.00 | 27.74 | 6 |
| ATOM | 5224 | CD1 | ILE | A | 954 | 80.781 | 43.691 | 87.966 | 1.00 | 33.57 | 6 |
| ATOM | 5225 | C | ILE | A | 954 | 79.792 | 47.036 | 89.224 | 1.00 | 24.45 | 6 |
| ATOM | 5226 | O | ILE | A | 954 | 79.550 | 47.700 | 90.228 | 1.00 | 26.39 | 8 |
| ATOM | 5227 | N | THR | A | 955 | 78.860 | 46.714 | 88.339 | 1.00 | 24.02 | 7 |
| ATOM | 5228 | CA | THR | A | 955 | 77.485 | 47.100 | 88.552 | 1.00 | 26.11 | 6 |
| ATOM | 5229 | CB | THR | A | 955 | 76.735 | 47.319 | 87.224 | 1.00 | 28.11 | 6 |
| ATOM | 5230 | OG1 | THR | A | 955 | 76.639 | 46.070 | 86.523 | 1.00 | 25.35 | 8 |
| ATOM | 5231 | CG2 | THR | A | 955 | 77.464 | 48.357 | 86.366 | 1.00 | 23.16 | 6 |
| ATOM | 5232 | C | THR | A | 955 | 76.792 | 45.978 | 89.310 | 1.00 | 27.93 | 6 |
| ATOM | 5233 | O | THR | A | 955 | 77.271 | 44.842 | 89.345 | 1.00 | 26.88 | 8 |
| ATOM | 5234 | N | GLU | A | 956 | 75.662 | 46.299 | 89.921 | 1.00 | 28.76 | 7 |
| ATOM | 5235 | CA | GLU | A | 956 | 74.937 | 45.295 | 90.665 | 1.00 | 31.90 | 6 |
| ATOM | 5236 | CB | GLU | A | 956 | 73.803 | 45.944 | 91.455 | 1.00 | 33.98 | 6 |
| ATOM | 5237 | CG | GLU | A | 956 | 74.250 | 47.245 | 92.126 | 1.00 | 36.40 | 6 |
| ATOM | 5238 | CD | GLU | A | 956 | 73.114 | 47.977 | 92.803 | 1.00 | 38.59 | 6 |
| ATOM | 5239 | OE1 | GLU | A | 956 | 72.188 | 47.300 | 93.297 | 1.00 | 40.22 | 8 |
| ATOM | 5240 | OE2 | GLU | A | 956 | 73.153 | 49.226 | 92.850 | 1.00 | 40.59 | 8 |
| ATOM | 5241 | C | GLU | A | 956 | 74.422 | 44.233 | 89.720 | 1.00 | 32.86 | 6 |
| ATOM | 5242 | O | GLU | A | 956 | 74.083 | 43.139 | 90.149 | 1.00 | 32.13 | 8 |
| ATOM | 5243 | N | THR | A | 957 | 74.380 | 44.547 | 88.427 | 1.00 | 31.31 | 7 |
| ATOM | 5244 | CA | THR | A | 957 | 73.932 | 43.564 | 87.456 | 1.00 | 32.42 | 6 |
| ATOM | 5245 | CB | THR | A | 957 | 73.203 | 44.223 | 86.286 | 1.00 | 33.93 | 6 |
| ATOM | 5246 | OG1 | THR | A | 957 | 74.097 | 45.107 | 85.598 | 1.00 | 40.17 | 8 |
| ATOM | 5247 | CG2 | THR | A | 957 | 71.995 | 45.001 | 86.784 | 1.00 | 32.42 | 6 |
| ATOM | 5248 | C | THR | A | 957 | 75.106 | 42.753 | 86.938 | 1.00 | 31.82 | 6 |
| ATOM | 5249 | O | THR | A | 957 | 74.982 | 42.011 | 85.959 | 1.00 | 33.39 | 8 |
| ATOM | 5250 | N | GLY | A | 958 | 76.262 | 42.898 | 87.585 | 1.00 | 30.12 | 7 |
| ATOM | 5251 | CA | GLY | A | 958 | 77.442 | 42.131 | 87.219 | 1.00 | 28.78 | 6 |
| ATOM | 5252 | C | GLY | A | 958 | 78.403 | 42.666 | 86.175 | 1.00 | 28.21 | 6 |
| ATOM | 5253 | O | GLY | A | 958 | 79.419 | 42.032 | 85.894 | 1.00 | 27.30 | 8 |
| ATOM | 5254 | N | ASN | A | 959 | 78.112 | 43.830 | 85.607 | 1.00 | 26.26 | 7 |
| ATOM | 5255 | CA | ASN | A | 959 | 78.982 | 44.390 | 84.577 | 1.00 | 24.88 | 6 |
| ATOM | 5256 | CB | ASN | A | 959 | 78.218 | 45.436 | 83.761 | 1.00 | 25.27 | 6 |
| ATOM | 5257 | CG | ASN | A | 959 | 78.691 | 45.514 | 82.329 | 1.00 | 27.14 | 6 |
| ATOM | 5258 | OD1 | ASN | A | 959 | 79.889 | 45.609 | 82.054 | 1.00 | 28.62 | 8 |
| ATOM | 5259 | ND2 | ASN | A | 959 | 77.744 | 45.473 | 81.401 | 1.00 | 25.02 | 7 |
| ATOM | 5260 | C | ASN | A | 959 | 80.248 | 45.037 | 85.128 | 1.00 | 23.70 | 6 |
| ATOM | 5261 | O | ASN | A | 959 | 80.188 | 46.082 | 85.784 | 1.00 | 22.39 | 8 |
| ATOM | 5262 | N | LEU | A | 960 | 81.397 | 44.429 | 84.867 | 1.00 | 23.25 | 7 |
| ATOM | 5263 | CA | LEU | A | 960 | 82.650 | 45.020 | 85.324 | 1.00 | 25.46 | 6 |
| ATOM | 5264 | CB | LEU | A | 960 | 83.747 | 43.970 | 85.393 | 1.00 | 27.15 | 6 |
| ATOM | 5265 | CG | LEU | A | 960 | 85.042 | 44.526 | 85.977 | 1.00 | 28.10 | 6 |
| ATOM | 5266 | CD1 | LEU | A | 960 | 85.569 | 43.564 | 87.020 | 1.00 | 30.12 | 6 |
| ATOM | 5267 | CD2 | LEU | A | 960 | 86.060 | 44.759 | 84.874 | 1.00 | 29.30 | 6 |
| ATOM | 5268 | C | LEU | A | 960 | 83.086 | 46.128 | 84.358 | 1.00 | 25.45 | 6 |
| ATOM | 5269 | O | LEU | A | 960 | 82.721 | 46.104 | 83.187 | 1.00 | 27.12 | 8 |
| ATOM | 5270 | N | PHE | A | 961 | 83.838 | 47.105 | 84.853 | 1.00 | 22.85 | 7 |
| ATOM | 5271 | CA | PHE | A | 961 | 84.342 | 48.178 | 84.008 | 1.00 | 23.55 | 6 |
| ATOM | 5272 | CB | PHE | A | 961 | 83.192 | 49.057 | 83.484 | 1.00 | 23.57 | 6 |
| ATOM | 5273 | CG | PHE | A | 961 | 82.620 | 50.019 | 84.497 | 1.00 | 26.43 | 6 |
| ATOM | 5274 | CD1 | PHE | A | 961 | 82.944 | 51.367 | 84.452 | 1.00 | 24.70 | 6 |
| ATOM | 5275 | CD2 | PHE | A | 961 | 81.702 | 49.587 | 85.451 | 1.00 | 26.34 | 6 |
| ATOM | 5276 | CE1 | PHE | A | 961 | 82.356 | 52.279 | 85.338 | 1.00 | 28.62 | 6 |
| ATOM | 5277 | CE2 | PHE | A | 961 | 81.110 | 50.482 | 86.338 | 1.00 | 28.56 | 6 |
| ATOM | 5278 | CZ | PHE | A | 961 | 81.433 | 51.836 | 86.283 | 1.00 | 28.26 | 6 |
| ATOM | 5279 | C | PHE | A | 961 | 85.402 | 49.026 | 84.707 | 1.00 | 23.47 | 6 |
| ATOM | 5280 | O | PHE | A | 961 | 85.288 | 49.323 | 85.899 | 1.00 | 24.79 | 8 |
| ATOM | 5281 | N | HIS | A | 962 | 86.450 | 49.373 | 83.958 | 1.00 | 22.83 | 7 |
| ATOM | 5282 | CA | HIS | A | 962 | 87.551 | 50.184 | 84.463 | 1.00 | 22.12 | 6 |
| ATOM | 5283 | CB | HIS | A | 962 | 88.796 | 49.976 | 83.611 | 1.00 | 22.89 | 6 |
| ATOM | 5284 | CG | HIS | A | 962 | 89.489 | 48.627 | 83.912 | 1.00 | 22.12 | 6 |
| ATOM | 5285 | CD2 | HIS | A | 962 | 88.976 | 47.393 | 84.136 | 1.00 | 25.81 | 6 |
| ATOM | 5286 | ND1 | HIS | A | 962 | 90.857 | 48.493 | 84.004 | 1.00 | 22.00 | 7 |
| ATOM | 5287 | CE1 | HIS | A | 962 | 91.161 | 47.235 | 84.278 | 1.00 | 23.56 | 6 |
| ATOM | 5288 | NE2 | HIS | A | 962 | 90.038 | 46.545 | 84.363 | 1.00 | 22.35 | 7 |
| ATOM | 5289 | C | HIS | A | 962 | 87.203 | 51.666 | 84.411 | 1.00 | 23.07 | 6 |
| ATOM | 5290 | O | HIS | A | 962 | 86.367 | 52.086 | 83.611 | 1.00 | 22.28 | 8 |
| ATOM | 5291 | N | ILE | A | 963 | 87.847 | 52.459 | 85.265 | 1.00 | 21.24 | 7 |
| ATOM | 5292 | CA | ILE | A | 963 | 87.626 | 53.900 | 85.291 | 1.00 | 23.70 | 6 |
| ATOM | 5293 | CB | ILE | A | 963 | 86.638 | 54.307 | 86.416 | 1.00 | 24.96 | 6 |
| ATOM | 5294 | CG2 | ILE | A | 963 | 85.259 | 53.710 | 86.179 | 1.00 | 21.54 | 6 |
| ATOM | 5295 | CG1 | ILE | A | 963 | 87.167 | 53.893 | 87.791 | 1.00 | 24.91 | 6 |
| ATOM | 5296 | CD1 | ILE | A | 963 | 86.501 | 54.624 | 88.939 | 1.00 | 25.79 | 6 |
| ATOM | 5297 | C | ILE | A | 963 | 88.909 | 54.665 | 85.588 | 1.00 | 23.18 | 6 |
| ATOM | 5298 | O | ILE | A | 963 | 89.953 | 54.077 | 85.819 | 1.00 | 22.10 | 8 |
| ATOM | 5299 | N | ASP | A | 964 | 88.772 | 56.003 | 85.584 | 1.00 | 27.31 | 7 |
| ATOM | 5300 | CA | ASP | A | 964 | 89.853 | 56.909 | 85.984 | 1.00 | 28.48 | 6 |
| ATOM | 5301 | CB | ASP | A | 964 | 90.107 | 56.672 | 87.461 | 1.00 | 31.30 | 6 |
| ATOM | 5302 | CG | ASP | A | 964 | 90.800 | 57.808 | 88.169 | 1.00 | 39.52 | 6 |
| ATOM | 5303 | OD1 | ASP | A | 964 | 90.921 | 57.735 | 89.402 | 1.00 | 37.68 | 8 |
| ATOM | 5304 | OD2 | ASP | A | 964 | 91.232 | 58.777 | 87.499 | 1.00 | 45.25 | 8 |
| ATOM | 5305 | C | ASP | A | 964 | 91.166 | 56.724 | 85.169 | 1.00 | 29.96 | 6 |
| ATOM | 5306 | O | ASP | A | 964 | 92.262 | 56.615 | 85.722 | 1.00 | 26.26 | 8 |
| ATOM | 5307 | N | PHE | A | 965 | 91.068 | 56.618 | 83.848 | 1.00 | 30.89 | 7 |
| ATOM | 5308 | CA | PHE | A | 965 | 92.185 | 56.271 | 82.960 | 1.00 | 33.06 | 6 |
| ATOM | 5309 | CB | PHE | A | 965 | 91.681 | 56.169 | 81.534 | 1.00 | 30.23 | 6 |
| ATOM | 5310 | CG | PHE | A | 965 | 90.696 | 55.103 | 81.433 | 1.00 | 27.01 | 6 |
| ATOM | 5311 | CD1 | PHE | A | 965 | 89.342 | 55.395 | 81.356 | 1.00 | 24.78 | 6 |
| ATOM | 5312 | CD2 | PHE | A | 965 | 91.090 | 53.774 | 81.478 | 1.00 | 24.52 | 6 |
| ATOM | 5313 | CE1 | PHE | A | 965 | 88.396 | 54.384 | 81.325 | 1.00 | 25.44 | 6 |
| ATOM | 5314 | CE2 | PHE | A | 965 | 90.154 | 52.760 | 81.480 | 1.00 | 23.93 | 6 |
| ATOM | 5315 | CZ | PHE | A | 965 | 88.801 | 53.063 | 81.404 | 1.00 | 23.17 | 6 |
| ATOM | 5316 | C | PHE | A | 965 | 93.395 | 57.182 | 82.825 | 1.00 | 36.41 | 6 |
| ATOM | 5317 | O | PHE | A | 965 | 93.551 | 57.744 | 81.734 | 1.00 | 35.82 | 8 |
| ATOM | 5318 | N | GLY | A | 966 | 94.278 | 57.381 | 83.786 | 1.00 | 107.63 | 7 |
| ATOM | 5319 | CA | GLY | A | 966 | 95.331 | 58.319 | 83.442 | 1.00 | 110.99 | 6 |
| ATOM | 5320 | C | GLY | A | 966 | 96.660 | 57.683 | 83.015 | 1.00 | 113.94 | 6 |
| ATOM | 5321 | O | GLY | A | 966 | 97.690 | 57.996 | 83.627 | 1.00 | 113.72 | 8 |
| ATOM | 5322 | N | HIS | A | 967 | 96.743 | 56.793 | 81.998 | 1.00 | 97.03 | 7 |
| ATOM | 5323 | CA | HIS | A | 967 | 98.114 | 56.229 | 81.722 | 1.00 | 99.97 | 6 |
| ATOM | 5324 | CB | HIS | A | 967 | 98.545 | 55.427 | 83.005 | 1.00 | 113.91 | 6 |
| ATOM | 5325 | CG | HIS | A | 967 | 99.053 | 53.936 | 82.957 | 1.00 | 115.45 | 6 |
| ATOM | 5326 | CD2 | HIS | A | 967 | 99.316 | 53.103 | 81.919 | 1.00 | 117.36 | 6 |
| ATOM | 5327 | ND1 | HIS | A | 967 | 99.340 | 53.158 | 84.093 | 1.00 | 116.31 | 7 |
| ATOM | 5328 | CE1 | HIS | A | 967 | 99.765 | 51.949 | 83.722 | 1.00 | 117.98 | 6 |
| ATOM | 5329 | NE2 | HIS | A | 967 | 99.769 | 51.901 | 82.409 | 1.00 | 118.42 | 7 |
| ATOM | 5330 | C | HIS | A | 967 | 98.391 | 55.365 | 80.474 | 1.00 | 100.39 | 6 |
| ATOM | 5331 | O | HIS | A | 967 | 97.769 | 54.304 | 80.302 | 1.00 | 101.31 | 8 |
| ATOM | 5332 | N | ALA | A | 968 | 99.327 | 55.793 | 79.601 | 1.00 | 66.10 | 7 |
| ATOM | 5333 | CA | ALA | A | 968 | 99.726 | 55.044 | 78.402 | 1.00 | 65.86 | 6 |
| ATOM | 5334 | CB | ALA | A | 968 | 100.403 | 53.739 | 78.809 | 1.00 | 56.44 | 6 |
| ATOM | 5335 | C | ALA | A | 968 | 98.546 | 54.746 | 77.487 | 1.00 | 66.34 | 6 |
| ATOM | 5336 | O | ALA | A | 968 | 98.143 | 55.583 | 76.684 | 1.00 | 65.65 | 8 |
| ATOM | 5337 | N | GLU | A | 981 | 106.981 | 44.356 | 90.596 | 1.00 | 51.73 | 7 |
| ATOM | 5338 | CA | GLU | A | 981 | 106.190 | 43.301 | 89.974 | 1.00 | 51.07 | 6 |
| ATOM | 5339 | CB | GLU | A | 981 | 105.736 | 42.290 | 91.025 | 1.00 | 52.96 | 6 |
| ATOM | 5340 | CG | GLU | A | 981 | 106.888 | 41.529 | 91.657 | 1.00 | 56.80 | 6 |
| ATOM | 5341 | CD | GLU | A | 981 | 106.422 | 40.348 | 92.478 | 1.00 | 58.68 | 6 |
| ATOM | 5342 | OE1 | GLU | A | 981 | 105.840 | 40.564 | 93.565 | 1.00 | 60.29 | 8 |
| ATOM | 5343 | OE2 | GLU | A | 981 | 106.633 | 39.202 | 92.028 | 1.00 | 60.44 | 8 |
| ATOM | 5344 | C | GLU | A | 981 | 104.981 | 43.874 | 89.248 | 1.00 | 49.92 | 6 |
| ATOM | 5345 | O | GLU | A | 981 | 104.390 | 44.862 | 89.693 | 1.00 | 47.26 | 8 |
| ATOM | 5346 | N | ARG | A | 982 | 104.614 | 43.244 | 88.133 | 1.00 | 48.49 | 7 |
| ATOM | 5347 | CA | ARG | A | 982 | 103.491 | 43.707 | 87.329 | 1.00 | 47.50 | 6 |
| ATOM | 5348 | CB | ARG | A | 982 | 104.020 | 44.527 | 86.146 | 1.00 | 50.63 | 6 |
| ATOM | 5349 | CG | ARG | A | 982 | 105.060 | 45.580 | 86.539 | 1.00 | 55.18 | 6 |
| ATOM | 5350 | CD | ARG | A | 982 | 104.468 | 46.660 | 87.449 | 1.00 | 59.70 | 6 |
| ATOM | 5351 | NE | ARG | A | 982 | 105.321 | 46.966 | 88.602 | 1.00 | 62.10 | 7 |
| ATOM | 5352 | CZ | ARG | A | 982 | 106.507 | 47.564 | 88.535 | 1.00 | 63.24 | 6 |
| ATOM | 5353 | NH1 | ARG | A | 982 | 107.007 | 47.938 | 87.363 | 1.00 | 64.19 | 7 |
| ATOM | 5354 | NH2 | ARG | A | 982 | 107.194 | 47.788 | 89.646 | 1.00 | 62.79 | 7 |
| ATOM | 5355 | C | ARG | A | 982 | 102.608 | 42.567 | 86.820 | 1.00 | 44.65 | 6 |
| ATOM | 5356 | O | ARG | A | 982 | 102.935 | 41.388 | 86.971 | 1.00 | 42.55 | 8 |
| ATOM | 5357 | N | VAL | A | 983 | 101.454 | 42.967 | 86.216 | 1.00 | 41.69 | 7 |
| ATOM | 5358 | CA | VAL | A | 983 | 100.438 | 42.036 | 85.649 | 1.00 | 40.15 | 6 |
| ATOM | 5359 | CB | VAL | A | 983 | 99.361 | 41.743 | 86.711 | 1.00 | 41.03 | 6 |
| ATOM | 5360 | CG1 | VAL | A | 983 | 98.375 | 42.900 | 86.794 | 1.00 | 39.85 | 6 |
| ATOM | 5361 | CG2 | VAL | A | 983 | 98.640 | 40.439 | 86.394 | 1.00 | 42.54 | 6 |
| ATOM | 5362 | C | VAL | A | 983 | 99.798 | 42.504 | 84.320 | 1.00 | 39.78 | 6 |
| ATOM | 5363 | O | VAL | A | 983 | 99.600 | 43.694 | 84.094 | 1.00 | 40.12 | 8 |
| ATOM | 5364 | N | PRO | A | 984 | 99.457 | 41.535 | 83.412 | 1.00 | 37.60 | 7 |
| ATOM | 5365 | CD | PRO | A | 984 | 100.350 | 40.406 | 83.113 | 1.00 | 36.05 | 6 |
| ATOM | 5366 | CA | PRO | A | 984 | 98.876 | 41.926 | 82.083 | 1.00 | 35.74 | 6 |
| ATOM | 5367 | CB | PRO | A | 984 | 98.634 | 40.603 | 81.392 | 1.00 | 35.60 | 6 |
| ATOM | 5368 | CG | PRO | A | 984 | 99.837 | 39.828 | 81.807 | 1.00 | 34.93 | 6 |
| ATOM | 5369 | C | PRO | A | 984 | 97.737 | 42.854 | 82.187 | 1.00 | 34.88 | 6 |
| ATOM | 5370 | O | PRO | A | 984 | 97.627 | 43.910 | 81.566 | 1.00 | 36.00 | 8 |
| ATOM | 5371 | N | PHE | A | 985 | 96.918 | 42.341 | 83.055 | 1.00 | 32.49 | 7 |
| ATOM | 5372 | CA | PHE | A | 985 | 95.662 | 42.895 | 83.390 | 1.00 | 29.82 | 6 |
| ATOM | 5373 | CB | PHE | A | 985 | 94.681 | 42.484 | 82.312 | 1.00 | 29.88 | 6 |
| ATOM | 5374 | CG | PHE | A | 985 | 94.814 | 41.046 | 81.834 | 1.00 | 30.38 | 6 |
| ATOM | 5375 | CD1 | PHE | A | 985 | 94.274 | 40.004 | 82.592 | 1.00 | 30.15 | 6 |
| ATOM | 5376 | CD2 | PHE | A | 985 | 95.433 | 40.743 | 80.627 | 1.00 | 28.89 | 6 |
| ATOM | 5377 | CE1 | PHE | A | 985 | 94.342 | 38.687 | 82.148 | 1.00 | 27.99 | 6 |
| ATOM | 5378 | CE2 | PHE | A | 985 | 95.505 | 39.429 | 80.174 | 1.00 | 27.92 | 6 |
| ATOM | 5379 | CZ | PHE | A | 985 | 94.957 | 38.400 | 80.936 | 1.00 | 28.92 | 6 |
| ATOM | 5380 | C | PHE | A | 985 | 95.190 | 42.418 | 84.752 | 1.00 | 29.59 | 6 |
| ATOM | 5381 | O | PHE | A | 985 | 95.902 | 41.682 | 85.430 | 1.00 | 28.68 | 8 |
| ATOM | 5382 | N | VAL | A | 986 | 93.999 | 42.841 | 85.153 | 1.00 | 28.98 | 7 |
| ATOM | 5383 | CA | VAL | A | 986 | 93.469 | 42.474 | 86.451 | 1.00 | 27.73 | 6 |
| ATOM | 5384 | CB | VAL | A | 986 | 92.747 | 43.685 | 87.110 | 1.00 | 28.45 | 6 |
| ATOM | 5385 | CG1 | VAL | A | 986 | 92.163 | 43.290 | 88.440 | 1.00 | 26.12 | 6 |
| ATOM | 5386 | CG2 | VAL | A | 986 | 93.739 | 44.840 | 87.309 | 1.00 | 26.19 | 6 |
| ATOM | 5387 | C | VAL | A | 986 | 92.525 | 41.283 | 86.392 | 1.00 | 27.39 | 6 |
| ATOM | 5388 | O | VAL | A | 986 | 91.444 | 41.342 | 85.801 | 1.00 | 25.78 | 8 |
| ATOM | 5389 | N | LEU | A | 987 | 92.994 | 40.194 | 86.947 | 1.00 | 27.03 | 7 |
| ATOM | 5390 | CA | LEU | A | 987 | 92.285 | 38.956 | 87.027 | 1.00 | 28.50 | 6 |
| ATOM | 5391 | CB | LEU | A | 987 | 92.546 | 38.000 | 85.853 | 1.00 | 28.17 | 6 |
| ATOM | 5392 | CG | LEU | A | 987 | 91.821 | 36.661 | 85.899 | 1.00 | 27.63 | 6 |
| ATOM | 5393 | CD1 | LEU | A | 987 | 90.311 | 36.877 | 85.877 | 1.00 | 26.65 | 6 |
| ATOM | 5394 | CD2 | LEU | A | 987 | 92.271 | 35.769 | 84.751 | 1.00 | 28.75 | 6 |
| ATOM | 5395 | C | LEU | A | 987 | 92.779 | 38.404 | 88.314 | 1.00 | 28.89 | 6 |
| ATOM | 5396 | O | LEU | A | 987 | 93.756 | 37.676 | 88.401 | 1.00 | 24.90 | 8 |
| ATOM | 5397 | N | THR | A | 988 | 92.044 | 38.812 | 89.286 | 1.00 | 29.15 | 7 |
| ATOM | 5398 | CA | THR | A | 988 | 92.328 | 38.537 | 90.656 | 1.00 | 30.49 | 6 |
| ATOM | 5399 | CB | THR | A | 988 | 92.109 | 39.839 | 91.437 | 1.00 | 31.85 | 6 |
| ATOM | 5400 | OG1 | THR | A | 988 | 90.739 | 40.231 | 91.367 | 1.00 | 31.86 | 8 |
| ATOM | 5401 | CG2 | THR | A | 988 | 92.980 | 40.949 | 90.867 | 1.00 | 29.36 | 6 |
| ATOM | 5402 | C | THR | A | 988 | 91.491 | 37.352 | 91.173 | 1.00 | 30.12 | 6 |
| ATOM | 5403 | O | THR | A | 988 | 90.573 | 36.943 | 90.473 | 1.00 | 31.64 | 8 |
| ATOM | 5404 | N | PRO | A | 989 | 91.758 | 36.755 | 92.382 | 1.00 | 30.28 | 7 |
| ATOM | 5405 | CD | PRO | A | 989 | 93.103 | 36.370 | 92.819 | 1.00 | 28.64 | 6 |
| ATOM | 5406 | CA | PRO | A | 989 | 90.884 | 35.622 | 92.794 | 1.00 | 29.52 | 6 |
| ATOM | 5407 | CB | PRO | A | 989 | 91.437 | 35.219 | 94.158 | 1.00 | 30.96 | 6 |
| ATOM | 5408 | CG | PRO | A | 989 | 92.906 | 35.390 | 93.941 | 1.00 | 30.67 | 6 |
| ATOM | 5409 | C | PRO | A | 989 | 89.452 | 35.994 | 92.700 | 1.00 | 30.20 | 6 |
| ATOM | 5410 | O | PRO | A | 989 | 88.657 | 35.541 | 91.878 | 1.00 | 30.77 | 8 |
| ATOM | 5411 | N | ASP | A | 990 | 89.172 | 36.878 | 93.635 | 1.00 | 28.71 | 7 |
| ATOM | 5412 | CA | ASP | A | 990 | 87.847 | 37.350 | 93.806 | 1.00 | 29.74 | 6 |
| ATOM | 5413 | CB | ASP | A | 990 | 87.815 | 38.751 | 94.474 | 1.00 | 31.90 | 6 |
| ATOM | 5414 | CG | ASP | A | 990 | 88.553 | 39.857 | 93.741 | 1.00 | 35.47 | 6 |
| ATOM | 5415 | OD1 | ASP | A | 990 | 89.679 | 39.608 | 93.252 | 1.00 | 35.69 | 8 |
| ATOM | 5416 | OD2 | ASP | A | 990 | 88.016 | 40.986 | 93.644 | 1.00 | 37.34 | 8 |
| ATOM | 5417 | C | ASP | A | 990 | 87.145 | 37.300 | 92.469 | 1.00 | 30.20 | 6 |
| ATOM | 5418 | O | ASP | A | 990 | 85.984 | 36.910 | 92.415 | 1.00 | 29.80 | 8 |
| ATOM | 5419 | N | PHE | A | 991 | 87.821 | 37.696 | 91.384 | 1.00 | 30.30 | 7 |
| ATOM | 5420 | CA | PHE | A | 991 | 87.150 | 37.632 | 90.077 | 1.00 | 30.73 | 6 |
| ATOM | 5421 | CB | PHE | A | 991 | 87.990 | 38.332 | 88.988 | 1.00 | 29.28 | 6 |
| ATOM | 5422 | CG | PHE | A | 991 | 88.046 | 39.845 | 89.107 | 1.00 | 29.74 | 6 |
| ATOM | 5423 | CD1 | PHE | A | 991 | 87.586 | 40.499 | 90.245 | 1.00 | 29.62 | 6 |
| ATOM | 5424 | CD2 | PHE | A | 991 | 88.611 | 40.608 | 88.084 | 1.00 | 29.23 | 6 |
| ATOM | 5425 | CE1 | PHE | A | 991 | 87.693 | 41.899 | 90.367 | 1.00 | 29.60 | 6 |
| ATOM | 5426 | CE2 | PHE | A | 991 | 88.724 | 42.000 | 88.196 | 1.00 | 28.44 | 6 |
| ATOM | 5427 | CZ | PHE | A | 991 | 88.266 | 42.644 | 89.338 | 1.00 | 26.82 | 6 |
| ATOM | 5428 | C | PHE | A | 991 | 86.987 | 36.149 | 89.712 | 1.00 | 30.46 | 6 |
| ATOM | 5429 | O | PHE | A | 991 | 85.912 | 35.702 | 89.295 | 1.00 | 30.48 | 8 |
| ATOM | 5430 | N | LEU | A | 992 | 88.068 | 35.397 | 89.878 | 1.00 | 29.93 | 7 |
| ATOM | 5431 | CA | LEU | A | 992 | 88.070 | 33.971 | 89.579 | 1.00 | 31.60 | 6 |
| ATOM | 5432 | CB | LEU | A | 992 | 89.454 | 33.388 | 89.864 | 1.00 | 29.72 | 6 |
| ATOM | 5433 | CG | LEU | A | 992 | 90.612 | 34.226 | 89.321 | 1.00 | 31.61 | 6 |
| ATOM | 5434 | CD1 | LEU | A | 992 | 91.953 | 33.721 | 89.871 | 1.00 | 30.33 | 6 |
| ATOM | 5435 | CD2 | LEU | A | 992 | 90.586 | 34.170 | 87.801 | 1.00 | 32.06 | 6 |
| ATOM | 5436 | C | LEU | A | 992 | 87.005 | 33.228 | 90.383 | 1.00 | 32.29 | 6 |
| ATOM | 5437 | O | LEU | A | 992 | 86.279 | 32.383 | 89.841 | 1.00 | 32.10 | 8 |
| ATOM | 5438 | N | PHE | A | 993 | 86.903 | 33.550 | 91.670 | 1.00 | 33.79 | 7 |
| ATOM | 5439 | CA | PHE | A | 993 | 85.916 | 32.917 | 92.537 | 1.00 | 35.79 | 6 |
| ATOM | 5440 | CB | PHE | A | 993 | 85.944 | 33.547 | 93.933 | 1.00 | 39.33 | 6 |
| ATOM | 5441 | CG | PHE | A | 993 | 85.098 | 32.821 | 94.938 | 1.00 | 42.22 | 6 |
| ATOM | 5442 | CD1 | PHE | A | 993 | 85.622 | 31.763 | 95.673 | 1.00 | 44.43 | 6 |
| ATOM | 5443 | CD2 | PHE | A | 993 | 83.764 | 33.172 | 95.127 | 1.00 | 44.41 | 6 |
| ATOM | 5444 | CE1 | PHE | A | 993 | 84.832 | 31.060 | 96.580 | 1.00 | 45.21 | 6 |
| ATOM | 5445 | CE2 | PHE | A | 993 | 82.962 | 32.478 | 96.031 | 1.00 | 45.86 | 6 |
| ATOM | 5446 | CZ | PHE | A | 993 | 83.498 | 31.419 | 96.760 | 1.00 | 46.64 | 6 |
| ATOM | 5447 | C | PHE | A | 993 | 84.520 | 33.071 | 91.937 | 1.00 | 36.11 | 6 |
| ATOM | 5448 | O | PHE | A | 993 | 83.724 | 32.134 | 91.954 | 1.00 | 35.03 | 8 |
| ATOM | 5449 | N | VAL | A | 994 | 84.216 | 34.258 | 91.415 | 1.00 | 37.13 | 7 |
| ATOM | 5450 | CA | VAL | A | 994 | 82.911 | 34.492 | 90.800 | 1.00 | 36.74 | 6 |
| ATOM | 5451 | CB | VAL | A | 994 | 82.799 | 35.939 | 90.257 | 1.00 | 37.53 | 6 |
| ATOM | 5452 | CG1 | VAL | A | 994 | 81.477 | 36.125 | 89.518 | 1.00 | 36.82 | 6 |
| ATOM | 5453 | CG2 | VAL | A | 994 | 82.909 | 36.940 | 91.407 | 1.00 | 36.38 | 6 |
| ATOM | 5454 | C | VAL | A | 994 | 82.730 | 33.505 | 89.638 | 1.00 | 38.92 | 6 |
| ATOM | 5455 | O | VAL | A | 994 | 81.618 | 33.045 | 89.350 | 1.00 | 37.08 | 8 |
| ATOM | 5456 | N | MET | A | 995 | 83.837 | 33.174 | 88.985 | 1.00 | 38.83 | 7 |
| ATOM | 5457 | CA | MET | A | 995 | 83.809 | 32.260 | 87.857 | 1.00 | 41.43 | 6 |
| ATOM | 5458 | CB | MET | A | 995 | 85.029 | 32.507 | 86.965 | 1.00 | 42.16 | 6 |
| ATOM | 5459 | CG | MET | A | 995 | 85.166 | 33.961 | 86.527 | 1.00 | 43.41 | 6 |
| ATOM | 5460 | SD | MET | A | 995 | 86.420 | 34.258 | 85.261 | 1.00 | 43.84 | 16 |
| ATOM | 5461 | CE | MET | A | 995 | 86.677 | 35.969 | 85.522 | 1.00 | 44.97 | 6 |
| ATOM | 5462 | C | MET | A | 995 | 83.771 | 30.807 | 88.330 | 1.00 | 43.41 | 6 |
| ATOM | 5463 | O | MET | A | 995 | 83.724 | 29.881 | 87.517 | 1.00 | 42.52 | 8 |
| ATOM | 5464 | N | GLY | A | 996 | 83.795 | 30.622 | 89.648 | 1.00 | 45.12 | 7 |
| ATOM | 5465 | CA | GLY | A | 996 | 83.742 | 29.288 | 90.220 | 1.00 | 47.36 | 6 |
| ATOM | 5466 | C | GLY | A | 996 | 85.049 | 28.520 | 90.346 | 1.00 | 48.76 | 6 |
| ATOM | 5467 | O | GLY | A | 996 | 85.028 | 27.329 | 90.651 | 1.00 | 47.76 | 8 |
| ATOM | 5468 | N | THR | A | 997 | 86.183 | 29.180 | 90.090 | 1.00 | 50.76 | 7 |
| ATOM | 5469 | CA | THR | A | 997 | 87.449 | 28.485 | 90.189 | 1.00 | 54.25 | 6 |
| ATOM | 5470 | CB | THR | A | 997 | 88.256 | 28.609 | 88.881 | 1.00 | 56.50 | 6 |
| ATOM | 5471 | OG1 | THR | A | 997 | 87.492 | 28.089 | 87.791 | 1.00 | 57.73 | 8 |
| ATOM | 5472 | CG2 | THR | A | 997 | 89.562 | 27.828 | 88.994 | 1.00 | 57.63 | 6 |
| ATOM | 5473 | C | THR | A | 997 | 88.239 | 29.077 | 91.357 | 1.00 | 55.25 | 6 |
| ATOM | 5474 | O | THR | A | 997 | 88.057 | 30.257 | 91.703 | 1.00 | 54.96 | 8 |
| ATOM | 5475 | N | SER | A | 998 | 89.122 | 28.292 | 91.983 | 1.00 | 56.94 | 7 |
| ATOM | 5476 | CA | SER | A | 998 | 89.936 | 28.788 | 93.107 | 1.00 | 59.15 | 6 |
| ATOM | 5477 | CB | SER | A | 998 | 89.086 | 28.798 | 94.367 | 1.00 | 59.32 | 6 |
| ATOM | 5478 | OG | SER | A | 998 | 89.552 | 27.839 | 95.300 | 1.00 | 62.07 | 8 |
| ATOM | 5479 | C | SER | A | 998 | 91.251 | 28.051 | 93.390 | 1.00 | 60.57 | 6 |
| ATOM | 5480 | O | SER | A | 998 | 91.289 | 27.196 | 94.256 | 1.00 | 60.99 | 8 |
| ATOM | 5481 | N | GLY | A | 999 | 92.339 | 28.363 | 92.677 | 1.00 | 62.22 | 7 |
| ATOM | 5482 | CA | GLY | A | 999 | 93.618 | 27.713 | 92.953 | 1.00 | 65.00 | 6 |
| ATOM | 5483 | C | GLY | A | 999 | 94.409 | 27.491 | 91.690 | 1.00 | 66.89 | 6 |
| ATOM | 5484 | O | GLY | A | 999 | 95.339 | 28.219 | 91.372 | 1.00 | 67.58 | 8 |
| ATOM | 5485 | N | LYS | A | 1000 | 94.023 | 26.483 | 90.987 | 1.00 | 68.03 | 7 |
| ATOM | 5486 | CA | LYS | A | 1000 | 94.580 | 26.107 | 89.703 | 1.00 | 69.47 | 6 |
| ATOM | 5487 | CB | LYS | A | 1000 | 96.064 | 25.757 | 89.715 | 1.00 | 70.59 | 6 |
| ATOM | 5488 | CG | LYS | A | 1000 | 96.852 | 26.670 | 88.776 | 1.00 | 72.51 | 6 |
| ATOM | 5489 | CD | LYS | A | 1000 | 97.217 | 27.978 | 89.460 | 1.00 | 73.37 | 6 |
| ATOM | 5490 | CE | LYS | A | 1000 | 98.437 | 27.843 | 90.369 | 1.00 | 73.29 | 6 |
| ATOM | 5491 | NZ | LYS | A | 1000 | 98.839 | 29.158 | 90.941 | 1.00 | 73.66 | 7 |
| ATOM | 5492 | C | LYS | A | 1000 | 93.727 | 24.976 | 89.327 | 1.00 | 69.25 | 6 |
| ATOM | 5493 | O | LYS | A | 1000 | 94.072 | 23.803 | 89.293 | 1.00 | 69.64 | 8 |
| ATOM | 5494 | N | LYS | A | 1001 | 92.558 | 25.460 | 89.064 | 1.00 | 68.55 | 7 |
| ATOM | 5495 | CA | LYS | A | 1001 | 91.522 | 24.594 | 88.754 | 1.00 | 66.95 | 6 |
| ATOM | 5496 | CB | LYS | A | 1001 | 90.386 | 24.753 | 89.757 | 1.00 | 68.10 | 6 |
| ATOM | 5497 | CG | LYS | A | 1001 | 90.376 | 23.694 | 90.849 | 1.00 | 68.79 | 6 |
| ATOM | 5498 | CD | LYS | A | 1001 | 89.669 | 24.195 | 92.078 | 1.00 | 69.91 | 6 |
| ATOM | 5499 | CE | LYS | A | 1001 | 88.358 | 23.464 | 92.292 | 1.00 | 70.18 | 6 |
| ATOM | 5500 | NZ | LYS | A | 1001 | 87.172 | 24.289 | 91.911 | 1.00 | 71.09 | 7 |
| ATOM | 5501 | C | LYS | A | 1001 | 90.978 | 24.835 | 87.410 | 1.00 | 65.00 | 6 |
| ATOM | 5502 | O | LYS | A | 1001 | 91.648 | 24.920 | 86.397 | 1.00 | 65.19 | 8 |
| ATOM | 5503 | N | THR | A | 1002 | 89.742 | 24.924 | 87.533 | 1.00 | 61.66 | 7 |
| ATOM | 5504 | CA | THR | A | 1002 | 88.963 | 25.026 | 86.313 | 1.00 | 58.39 | 6 |
| ATOM | 5505 | CB | THR | A | 1002 | 89.279 | 23.873 | 85.360 | 1.00 | 59.54 | 6 |
| ATOM | 5506 | OG1 | THR | A | 1002 | 88.620 | 24.089 | 84.107 | 1.00 | 61.24 | 8 |
| ATOM | 5507 | CG2 | THR | A | 1002 | 88.827 | 22.549 | 85.957 | 1.00 | 59.77 | 6 |
| ATOM | 5508 | C | THR | A | 1002 | 87.473 | 25.058 | 86.621 | 1.00 | 55.30 | 6 |
| ATOM | 5509 | O | THR | A | 1002 | 87.054 | 24.718 | 87.720 | 1.00 | 56.42 | 8 |
| ATOM | 5510 | N | SER | A | 1003 | 86.710 | 25.446 | 85.613 | 1.00 | 51.09 | 7 |
| ATOM | 5511 | CA | SER | A | 1003 | 85.259 | 25.549 | 85.711 | 1.00 | 47.82 | 6 |
| ATOM | 5512 | CB | SER | A | 1003 | 84.875 | 26.558 | 86.798 | 1.00 | 47.37 | 6 |
| ATOM | 5513 | OG | SER | A | 1003 | 85.305 | 27.867 | 86.461 | 1.00 | 46.63 | 8 |
| ATOM | 5514 | C | SER | A | 1003 | 84.630 | 25.956 | 84.383 | 1.00 | 45.58 | 6 |
| ATOM | 5515 | O | SER | A | 1003 | 85.284 | 26.523 | 83.499 | 1.00 | 45.11 | 8 |
| ATOM | 5516 | N | PRO | A | 1004 | 83.343 | 25.665 | 84.250 | 1.00 | 43.39 | 7 |
| ATOM | 5517 | CD | PRO | A | 1004 | 82.843 | 24.326 | 84.630 | 1.00 | 42.06 | 6 |
| ATOM | 5518 | CA | PRO | A | 1004 | 82.622 | 26.063 | 83.021 | 1.00 | 41.41 | 6 |
| ATOM | 5519 | CB | PRO | A | 1004 | 81.231 | 25.525 | 83.256 | 1.00 | 42.02 | 6 |
| ATOM | 5520 | CG | PRO | A | 1004 | 81.513 | 24.214 | 83.950 | 1.00 | 43.96 | 6 |
| ATOM | 5521 | C | PRO | A | 1004 | 82.823 | 27.500 | 82.661 | 1.00 | 39.11 | 6 |
| ATOM | 5522 | O | PRO | A | 1004 | 83.103 | 27.858 | 81.530 | 1.00 | 37.60 | 8 |
| ATOM | 5523 | N | HIS | A | 1005 | 82.659 | 28.333 | 83.682 | 1.00 | 36.78 | 7 |
| ATOM | 5524 | CA | HIS | A | 1005 | 82.765 | 29.783 | 83.518 | 1.00 | 35.56 | 6 |
| ATOM | 5525 | CB | HIS | A | 1005 | 82.297 | 30.488 | 84.781 | 1.00 | 35.67 | 6 |
| ATOM | 5526 | CG | HIS | A | 1005 | 80.782 | 30.503 | 84.977 | 1.00 | 36.24 | 6 |
| ATOM | 5527 | CD2 | HIS | A | 1005 | 80.029 | 30.210 | 86.064 | 1.00 | 38.39 | 6 |
| ATOM | 5528 | ND1 | HIS | A | 1005 | 79.896 | 30.842 | 83.978 | 1.00 | 40.10 | 7 |
| ATOM | 5529 | CE1 | HIS | A | 1005 | 78.660 | 30.761 | 84.440 | 1.00 | 39.67 | 6 |
| ATOM | 5530 | NE2 | HIS | A | 1005 | 78.714 | 30.379 | 85.704 | 1.00 | 38.79 | 7 |
| ATOM | 5531 | C | HIS | A | 1005 | 84.156 | 30.221 | 83.183 | 1.00 | 34.58 | 6 |
| ATOM | 5532 | O | HIS | A | 1005 | 84.359 | 31.059 | 82.298 | 1.00 | 34.62 | 8 |
| ATOM | 5533 | N | PHE | A | 1006 | 85.146 | 29.669 | 83.880 | 1.00 | 31.65 | 7 |
| ATOM | 5534 | CA | PHE | A | 1006 | 86.520 | 30.054 | 83.592 | 1.00 | 32.27 | 6 |
| ATOM | 5535 | CB | PHE | A | 1006 | 87.509 | 29.389 | 84.552 | 1.00 | 35.31 | 6 |
| ATOM | 5536 | CG | PHE | A | 1006 | 88.944 | 29.747 | 84.272 | 1.00 | 37.62 | 6 |
| ATOM | 5537 | CD1 | PHE | A | 1006 | 89.382 | 31.067 | 84.392 | 1.00 | 39.64 | 6 |
| ATOM | 5538 | CD2 | PHE | A | 1006 | 89.855 | 28.772 | 83.870 | 1.00 | 39.21 | 6 |
| ATOM | 5539 | CE1 | PHE | A | 1006 | 90.708 | 31.411 | 84.116 | 1.00 | 41.57 | 6 |
| ATOM | 5540 | CE2 | PHE | A | 1006 | 91.182 | 29.099 | 83.590 | 1.00 | 38.36 | 6 |
| ATOM | 5541 | CZ | PHE | A | 1006 | 91.612 | 30.423 | 83.713 | 1.00 | 42.11 | 6 |
| ATOM | 5542 | C | PHE | A | 1006 | 86.867 | 29.666 | 82.164 | 1.00 | 32.65 | 6 |
| ATOM | 5543 | O | PHE | A | 1006 | 87.412 | 30.474 | 81.408 | 1.00 | 32.41 | 8 |
| ATOM | 5544 | N | GLN | A | 1007 | 86.556 | 28.424 | 81.792 | 1.00 | 31.71 | 7 |
| ATOM | 5545 | CA | GLN | A | 1007 | 86.827 | 27.973 | 80.437 | 1.00 | 32.34 | 6 |
| ATOM | 5546 | CB | GLN | A | 1007 | 86.365 | 26.520 | 80.252 | 1.00 | 34.57 | 6 |
| ATOM | 5547 | CG | GLN | A | 1007 | 87.107 | 25.525 | 81.128 | 1.00 | 38.89 | 6 |
| ATOM | 5548 | CD | GLN | A | 1007 | 86.848 | 24.079 | 80.727 | 1.00 | 42.63 | 6 |
| ATOM | 5549 | OE1 | GLN | A | 1007 | 87.362 | 23.157 | 81.353 | 1.00 | 44.52 | 8 |
| ATOM | 5550 | NE2 | GLN | A | 1007 | 86.054 | 23.880 | 79.673 | 1.00 | 43.10 | 7 |
| ATOM | 5551 | C | GLN | A | 1007 | 86.082 | 28.895 | 79.473 | 1.00 | 31.52 | 6 |
| ATOM | 5552 | O | GLN | A | 1007 | 86.581 | 29.220 | 78.399 | 1.00 | 30.63 | 8 |
| ATOM | 5553 | N | LYS | A | 1008 | 84.883 | 29.310 | 79.870 | 1.00 | 31.06 | 7 |
| ATOM | 5554 | CA | LYS | A | 1008 | 84.069 | 30.212 | 79.063 | 1.00 | 34.13 | 6 |
| ATOM | 5555 | CB | LYS | A | 1008 | 82.720 | 30.444 | 79.746 | 1.00 | 38.12 | 6 |
| ATOM | 5556 | CG | LYS | A | 1008 | 81.648 | 31.032 | 78.844 | 1.00 | 40.26 | 6 |
| ATOM | 5557 | CD | LYS | A | 1008 | 81.115 | 29.980 | 77.874 | 1.00 | 45.72 | 6 |
| ATOM | 5558 | CE | LYS | A | 1008 | 80.132 | 30.587 | 76.878 | 1.00 | 47.20 | 6 |
| ATOM | 5559 | NZ | LYS | A | 1008 | 79.501 | 29.563 | 75.995 | 1.00 | 48.34 | 7 |
| ATOM | 5560 | C | LYS | A | 1008 | 84.825 | 31.545 | 78.935 | 1.00 | 34.43 | 6 |
| ATOM | 5561 | O | LYS | A | 1008 | 84.821 | 32.177 | 77.873 | 1.00 | 32.44 | 8 |
| ATOM | 5562 | N | PHE | A | 1009 | 85.470 | 31.946 | 80.034 | 1.00 | 34.13 | 7 |
| ATOM | 5563 | CA | PHE | A | 1009 | 86.267 | 33.176 | 80.118 | 1.00 | 33.89 | 6 |
| ATOM | 5564 | CB | PHE | A | 1009 | 86.822 | 33.340 | 81.544 | 1.00 | 31.07 | 6 |
| ATOM | 5565 | CG | PHE | A | 1009 | 87.740 | 34.532 | 81.722 | 1.00 | 30.14 | 6 |
| ATOM | 5566 | CD1 | PHE | A | 1009 | 87.222 | 35.822 | 81.852 | 1.00 | 28.75 | 6 |
| ATOM | 5567 | CD2 | PHE | A | 1009 | 89.117 | 34.363 | 81.752 | 1.00 | 28.08 | 6 |
| ATOM | 5568 | CE1 | PHE | A | 1009 | 88.062 | 36.921 | 82.008 | 1.00 | 25.93 | 6 |
| ATOM | 5569 | CE2 | PHE | A | 1009 | 89.969 | 35.454 | 81.903 | 1.00 | 31.08 | 6 |
| ATOM | 5570 | CZ | PHE | A | 1009 | 89.434 | 36.741 | 82.032 | 1.00 | 30.47 | 6 |
| ATOM | 5571 | C | PHE | A | 1009 | 87.435 | 33.094 | 79.137 | 1.00 | 34.58 | 6 |
| ATOM | 5572 | O | PHE | A | 1009 | 87.664 | 34.007 | 78.342 | 1.00 | 34.74 | 8 |
| ATOM | 5573 | N | GLN | A | 1010 | 88.167 | 31.988 | 79.203 | 1.00 | 33.16 | 7 |
| ATOM | 5574 | CA | GLN | A | 1010 | 89.323 | 31.781 | 78.334 | 1.00 | 34.46 | 6 |
| ATOM | 5575 | CB | GLN | A | 1010 | 89.992 | 30.442 | 78.640 | 1.00 | 33.69 | 6 |
| ATOM | 5576 | CG | GLN | A | 1010 | 90.641 | 30.340 | 80.006 | 1.00 | 32.51 | 6 |
| ATOM | 5577 | CD | GLN | A | 1010 | 91.186 | 28.941 | 80.259 | 1.00 | 34.76 | 6 |
| ATOM | 5578 | OE1 | GLN | A | 1010 | 90.440 | 27.957 | 80.209 | 1.00 | 32.39 | 8 |
| ATOM | 5579 | NE2 | GLN | A | 1010 | 92.486 | 28.845 | 80.527 | 1.00 | 34.26 | 7 |
| ATOM | 5580 | C | GLN | A | 1010 | 88.967 | 31.813 | 76.858 | 1.00 | 33.02 | 6 |
| ATOM | 5581 | O | GLN | A | 1010 | 89.718 | 32.352 | 76.053 | 1.00 | 35.02 | 8 |
| ATOM | 5582 | N | ASP | A | 1011 | 87.826 | 31.226 | 76.511 | 1.00 | 33.84 | 7 |
| ATOM | 5583 | CA | ASP | A | 1011 | 87.369 | 31.164 | 75.122 | 1.00 | 33.16 | 6 |
| ATOM | 5584 | CB | ASP | A | 1011 | 86.195 | 30.192 | 75.003 | 1.00 | 35.01 | 6 |
| ATOM | 5585 | CG | ASP | A | 1011 | 85.730 | 30.015 | 73.566 | 1.00 | 37.85 | 6 |
| ATOM | 5586 | OD1 | ASP | A | 1011 | 86.599 | 29.877 | 72.680 | 1.00 | 41.16 | 8 |
| ATOM | 5587 | OD2 | ASP | A | 1011 | 84.506 | 30.001 | 73.322 | 1.00 | 40.48 | 8 |
| ATOM | 5588 | C | ASP | A | 1011 | 86.955 | 32.531 | 74.573 | 1.00 | 32.70 | 6 |
| ATOM | 5589 | O | ASP | A | 1011 | 87.262 | 32.880 | 73.429 | 1.00 | 30.83 | 8 |
| ATOM | 5590 | N | ILE | A | 1012 | 86.226 | 33.287 | 75.384 | 1.00 | 30.48 | 7 |
| ATOM | 5591 | CA | ILE | A | 1012 | 85.793 | 34.622 | 74.995 | 1.00 | 30.39 | 6 |
| ATOM | 5592 | CB | ILE | A | 1012 | 84.890 | 35.236 | 76.098 | 1.00 | 31.53 | 6 |
| ATOM | 5593 | CG2 | ILE | A | 1012 | 84.455 | 36.631 | 75.715 | 1.00 | 32.31 | 6 |
| ATOM | 5594 | CG1 | ILE | A | 1012 | 83.658 | 34.354 | 76.296 | 1.00 | 33.51 | 6 |
| ATOM | 5595 | CD1 | ILE | A | 1012 | 82.677 | 34.876 | 77.333 | 1.00 | 37.18 | 6 |
| ATOM | 5596 | C | ILE | A | 1012 | 87.041 | 35.490 | 74.786 | 1.00 | 28.24 | 6 |
| ATOM | 5597 | O | ILE | A | 1012 | 87.160 | 36.182 | 73.765 | 1.00 | 27.84 | 8 |
| ATOM | 5598 | N | CYS | A | 1013 | 87.971 | 35.444 | 75.742 | 1.00 | 27.17 | 7 |
| ATOM | 5599 | CA | CYS | A | 1013 | 89.211 | 36.228 | 75.655 | 1.00 | 27.49 | 6 |
| ATOM | 5600 | CB | CYS | A | 1013 | 90.145 | 35.916 | 76.828 | 1.00 | 26.31 | 6 |
| ATOM | 5601 | SG | CYS | A | 1013 | 89.807 | 36.774 | 78.387 | 1.00 | 28.87 | 16 |
| ATOM | 5602 | C | CYS | A | 1013 | 89.984 | 35.971 | 74.355 | 1.00 | 29.10 | 6 |
| ATOM | 5603 | O | CYS | A | 1013 | 90.356 | 36.911 | 73.641 | 1.00 | 25.44 | 8 |
| ATOM | 5604 | N | VAL | A | 1014 | 90.248 | 34.696 | 74.066 | 1.00 | 29.24 | 7 |
| ATOM | 5605 | CA | VAL | A | 1014 | 90.984 | 34.339 | 72.856 | 1.00 | 29.48 | 6 |
| ATOM | 5606 | CB | VAL | A | 1014 | 91.189 | 32.808 | 72.733 | 1.00 | 28.90 | 6 |
| ATOM | 5607 | CG1 | VAL | A | 1014 | 91.691 | 32.457 | 71.330 | 1.00 | 27.17 | 6 |
| ATOM | 5608 | CG2 | VAL | A | 1014 | 92.194 | 32.344 | 73.765 | 1.00 | 29.50 | 6 |
| ATOM | 5609 | C | VAL | A | 1014 | 90.288 | 34.831 | 71.598 | 1.00 | 28.98 | 6 |
| ATOM | 5610 | O | VAL | A | 1014 | 90.928 | 35.386 | 70.714 | 1.00 | 29.45 | 8 |
| ATOM | 5611 | N | LYS | A | 1015 | 88.982 | 34.619 | 71.515 | 1.00 | 31.22 | 7 |
| ATOM | 5612 | CA | LYS | A | 1015 | 88.228 | 35.041 | 70.344 | 1.00 | 33.01 | 6 |
| ATOM | 5613 | CB | LYS | A | 1015 | 86.798 | 34.506 | 70.408 | 1.00 | 36.59 | 6 |
| ATOM | 5614 | CG | LYS | A | 1015 | 85.984 | 34.824 | 69.166 | 1.00 | 42.84 | 6 |
| ATOM | 5615 | CD | LYS | A | 1015 | 84.588 | 34.241 | 69.240 | 1.00 | 47.48 | 6 |
| ATOM | 5616 | CE | LYS | A | 1015 | 83.789 | 34.573 | 67.983 | 1.00 | 49.76 | 6 |
| ATOM | 5617 | NZ | LYS | A | 1015 | 82.353 | 34.167 | 68.113 | 1.00 | 52.04 | 7 |
| ATOM | 5618 | C | LYS | A | 1015 | 88.195 | 36.559 | 70.221 | 1.00 | 32.44 | 6 |
| ATOM | 5619 | O | LYS | A | 1015 | 88.235 | 37.100 | 69.118 | 1.00 | 31.70 | 8 |
| ATOM | 5620 | N | ALA | A | 1016 | 88.111 | 37.243 | 71.357 | 1.00 | 30.35 | 7 |
| ATOM | 5621 | CA | ALA | A | 1016 | 88.071 | 38.702 | 71.355 | 1.00 | 29.73 | 6 |
| ATOM | 5622 | CB | ALA | A | 1016 | 87.703 | 39.223 | 72.740 | 1.00 | 28.52 | 6 |
| ATOM | 5623 | C | ALA | A | 1016 | 89.427 | 39.240 | 70.948 | 1.00 | 27.57 | 6 |
| ATOM | 5624 | O | ALA | A | 1016 | 89.532 | 40.212 | 70.199 | 1.00 | 27.03 | 8 |
| ATOM | 5625 | N | TYR | A | 1017 | 90.469 | 38.590 | 71.444 | 1.00 | 25.18 | 7 |
| ATOM | 5626 | CA | TYR | A | 1017 | 91.834 | 39.007 | 71.162 | 1.00 | 27.03 | 6 |
| ATOM | 5627 | CB | TYR | A | 1017 | 92.796 | 38.264 | 72.087 | 1.00 | 25.14 | 6 |
| ATOM | 5628 | CG | TYR | A | 1017 | 94.264 | 38.558 | 71.869 | 1.00 | 25.98 | 6 |
| ATOM | 5629 | CD1 | TYR | A | 1017 | 94.773 | 39.852 | 72.018 | 1.00 | 26.31 | 6 |
| ATOM | 5630 | CE1 | TYR | A | 1017 | 96.125 | 40.114 | 71.855 | 1.00 | 26.50 | 6 |
| ATOM | 5631 | CD2 | TYR | A | 1017 | 95.151 | 37.536 | 71.546 | 1.00 | 23.86 | 6 |
| ATOM | 5632 | CE2 | TYR | A | 1017 | 96.502 | 37.785 | 71.374 | 1.00 | 27.13 | 6 |
| ATOM | 5633 | CZ | TYR | A | 1017 | 96.982 | 39.074 | 71.530 | 1.00 | 25.88 | 6 |
| ATOM | 5634 | OH | TYR | A | 1017 | 98.317 | 39.319 | 71.362 | 1.00 | 29.91 | 8 |
| ATOM | 5635 | C | TYR | A | 1017 | 92.233 | 38.798 | 69.702 | 1.00 | 27.27 | 6 |
| ATOM | 5636 | O | TYR | A | 1017 | 92.813 | 39.686 | 69.086 | 1.00 | 26.20 | 8 |
| ATOM | 5637 | N | LEU | A | 1018 | 91.938 | 37.630 | 69.152 | 1.00 | 28.76 | 7 |
| ATOM | 5638 | CA | LEU | A | 1018 | 92.306 | 37.434 | 67.746 | 1.00 | 31.26 | 6 |
| ATOM | 5639 | CB | LEU | A | 1018 | 92.184 | 35.956 | 67.358 | 1.00 | 35.13 | 6 |
| ATOM | 5640 | CG | LEU | A | 1018 | 93.058 | 34.970 | 68.136 | 1.00 | 38.53 | 6 |
| ATOM | 5641 | CD1 | LEU | A | 1018 | 92.827 | 33.547 | 67.650 | 1.00 | 40.26 | 6 |
| ATOM | 5642 | CD2 | LEU | A | 1018 | 94.527 | 35.344 | 68.016 | 1.00 | 39.15 | 6 |
| ATOM | 5643 | C | LEU | A | 1018 | 91.445 | 38.287 | 66.811 | 1.00 | 30.45 | 6 |
| ATOM | 5644 | O | LEU | A | 1018 | 91.879 | 38.678 | 65.740 | 1.00 | 30.48 | 8 |
| ATOM | 5645 | N | ALA | A | 1019 | 90.229 | 38.596 | 67.268 | 1.00 | 28.02 | 7 |
| ATOM | 5646 | CA | ALA | A | 1019 | 89.358 | 39.458 | 66.488 | 1.00 | 27.22 | 6 |
| ATOM | 5647 | CB | ALA | A | 1019 | 87.975 | 39.525 | 67.128 | 1.00 | 29.42 | 6 |
| ATOM | 5648 | C | ALA | A | 1019 | 89.978 | 40.870 | 66.403 | 1.00 | 28.33 | 6 |
| ATOM | 5649 | O | ALA | A | 1019 | 89.825 | 41.556 | 65.389 | 1.00 | 29.72 | 8 |
| ATOM | 5650 | N | LEU | A | 1020 | 90.667 | 41.312 | 67.459 | 1.00 | 27.82 | 7 |
| ATOM | 5651 | CA | LEU | A | 1020 | 91.297 | 42.638 | 67.434 | 1.00 | 27.27 | 6 |
| ATOM | 5652 | CB | LEU | A | 1020 | 91.730 | 43.082 | 68.851 | 1.00 | 23.37 | 6 |
| ATOM | 5653 | CG | LEU | A | 1020 | 90.625 | 43.398 | 69.873 | 1.00 | 24.73 | 6 |
| ATOM | 5654 | CD1 | LEU | A | 1020 | 91.231 | 44.066 | 71.095 | 1.00 | 27.48 | 6 |
| ATOM | 5655 | CD2 | LEU | A | 1020 | 89.581 | 44.316 | 69.263 | 1.00 | 25.81 | 6 |
| ATOM | 5656 | C | LEU | A | 1020 | 92.519 | 42.626 | 66.496 | 1.00 | 27.87 | 6 |
| ATOM | 5657 | O | LEU | A | 1020 | 92.768 | 43.592 | 65.756 | 1.00 | 26.53 | 8 |
| ATOM | 5658 | N | ARG | A | 1021 | 93.269 | 41.528 | 66.530 | 1.00 | 27.15 | 7 |
| ATOM | 5659 | CA | ARG | A | 1021 | 94.454 | 41.379 | 65.692 | 1.00 | 30.63 | 6 |
| ATOM | 5660 | CB | ARG | A | 1021 | 95.162 | 40.053 | 65.991 | 1.00 | 28.33 | 6 |
| ATOM | 5661 | CG | ARG | A | 1021 | 95.616 | 39.929 | 67.432 | 1.00 | 29.38 | 6 |
| ATOM | 5662 | CD | ARG | A | 1021 | 96.338 | 38.618 | 67.723 | 1.00 | 29.69 | 6 |
| ATOM | 5663 | NE | ARG | A | 1021 | 97.636 | 38.515 | 67.054 | 1.00 | 32.93 | 7 |
| ATOM | 5664 | CZ | ARG | A | 1021 | 97.861 | 37.800 | 65.952 | 1.00 | 32.82 | 6 |
| ATOM | 5665 | NH1 | ARG | A | 1021 | 96.876 | 37.117 | 65.388 | 1.00 | 31.06 | 7 |
| ATOM | 5666 | NH2 | ARG | A | 1021 | 99.074 | 37.766 | 65.411 | 1.00 | 31.93 | 7 |
| ATOM | 5667 | C | ARG | A | 1021 | 94.121 | 41.472 | 64.202 | 1.00 | 30.35 | 6 |
| ATOM | 5668 | O | ARG | A | 1021 | 94.985 | 41.810 | 63.400 | 1.00 | 33.33 | 8 |
| ATOM | 5669 | N | HIS | A | 1022 | 92.875 | 41.177 | 63.843 | 1.00 | 30.72 | 7 |
| ATOM | 5670 | CA | HIS | A | 1022 | 92.424 | 41.248 | 62.453 | 1.00 | 31.74 | 6 |
| ATOM | 5671 | CB | HIS | A | 1022 | 91.051 | 40.577 | 62.306 | 1.00 | 34.08 | 6 |
| ATOM | 5672 | CG | HIS | A | 1022 | 91.060 | 39.099 | 62.574 | 1.00 | 36.26 | 6 |
| ATOM | 5673 | CD2 | HIS | A | 1022 | 92.068 | 38.195 | 62.573 | 1.00 | 36.98 | 6 |
| ATOM | 5674 | ND1 | HIS | A | 1022 | 89.915 | 38.393 | 62.879 | 1.00 | 37.46 | 7 |
| ATOM | 5675 | CE1 | HIS | A | 1022 | 90.217 | 37.120 | 63.055 | 1.00 | 37.31 | 6 |
| ATOM | 5676 | NE2 | HIS | A | 1022 | 91.518 | 36.972 | 62.876 | 1.00 | 36.81 | 7 |
| ATOM | 5677 | C | HIS | A | 1022 | 92.313 | 42.715 | 62.041 | 1.00 | 32.13 | 6 |
| ATOM | 5678 | O | HIS | A | 1022 | 92.137 | 43.041 | 60.862 | 1.00 | 33.00 | 8 |
| ATOM | 5679 | N | HIS | A | 1023 | 92.412 | 43.593 | 63.031 | 1.00 | 29.82 | 7 |
| ATOM | 5680 | CA | HIS | A | 1023 | 92.322 | 45.022 | 62.839 | 1.00 | 29.80 | 6 |
| ATOM | 5681 | CB | HIS | A | 1023 | 91.078 | 45.591 | 63.490 | 1.00 | 29.42 | 6 |
| ATOM | 5682 | CG | HIS | A | 1023 | 89.795 | 45.090 | 62.788 | 1.00 | 28.10 | 6 |
| ATOM | 5683 | CD2 | HIS | A | 1023 | 89.102 | 43.931 | 62.904 | 1.00 | 25.68 | 6 |
| ATOM | 5684 | ND1 | HIS | A | 1023 | 89.150 | 45.829 | 61.820 | 1.00 | 26.18 | 7 |
| ATOM | 5685 | CE1 | HIS | A | 1023 | 88.112 | 45.147 | 61.368 | 1.00 | 25.30 | 6 |
| ATOM | 5686 | NE2 | HIS | A | 1023 | 88.061 | 43.992 | 62.008 | 1.00 | 27.61 | 7 |
| ATOM | 5687 | C | HIS | A | 1023 | 93.549 | 45.682 | 63.397 | 1.00 | 28.34 | 6 |
| ATOM | 5688 | O | HIS | A | 1023 | 93.490 | 46.804 | 63.904 | 1.00 | 28.44 | 8 |
| ATOM | 5689 | N | THR | A | 1024 | 94.662 | 44.964 | 63.286 | 1.00 | 26.33 | 7 |
| ATOM | 5690 | CA | THR | A | 1024 | 95.941 | 45.424 | 63.833 | 1.00 | 27.35 | 6 |
| ATOM | 5691 | CB | THR | A | 1024 | 97.057 | 44.411 | 63.489 | 1.00 | 29.67 | 6 |
| ATOM | 5692 | OG1 | THR | A | 1024 | 96.887 | 43.236 | 64.291 | 1.00 | 28.42 | 8 |
| ATOM | 5693 | CG2 | THR | A | 1024 | 98.437 | 45.011 | 63.747 | 1.00 | 30.75 | 6 |
| ATOM | 5694 | C | THR | A | 1024 | 96.387 | 46.814 | 63.383 | 1.00 | 28.06 | 6 |
| ATOM | 5695 | O | THR | A | 1024 | 96.827 | 47.613 | 64.202 | 1.00 | 26.98 | 8 |
| ATOM | 5696 | N | ASN | A | 1025 | 96.275 | 47.128 | 62.101 | 1.00 | 26.47 | 7 |
| ATOM | 5697 | CA | ASN | A | 1025 | 96.706 | 48.423 | 61.621 | 1.00 | 26.63 | 6 |
| ATOM | 5698 | CB | ASN | A | 1025 | 96.668 | 48.472 | 60.093 | 1.00 | 27.64 | 6 |
| ATOM | 5699 | CG | ASN | A | 1025 | 97.356 | 49.703 | 59.532 | 1.00 | 31.86 | 6 |
| ATOM | 5700 | OD1 | ASN | A | 1025 | 96.995 | 50.193 | 58.463 | 1.00 | 31.24 | 8 |
| ATOM | 5701 | ND2 | ASN | A | 1025 | 98.349 | 50.204 | 60.256 | 1.00 | 32.19 | 7 |
| ATOM | 5702 | C | ASN | A | 1025 | 95.875 | 49.557 | 62.212 | 1.00 | 24.99 | 6 |
| ATOM | 5703 | O | ASN | A | 1025 | 96.404 | 50.554 | 62.699 | 1.00 | 27.11 | 8 |
| ATOM | 5704 | N | LEU | A | 1026 | 94.568 | 49.389 | 62.188 | 1.00 | 25.99 | 7 |
| ATOM | 5705 | CA | LEU | A | 1026 | 93.648 | 50.418 | 62.669 | 1.00 | 25.06 | 6 |
| ATOM | 5706 | CB | LEU | A | 1026 | 92.210 | 49.849 | 62.698 | 1.00 | 24.98 | 6 |
| ATOM | 5707 | CG | LEU | A | 1026 | 91.061 | 50.867 | 62.627 | 1.00 | 28.89 | 6 |
| ATOM | 5708 | CD1 | LEU | A | 1026 | 89.887 | 50.403 | 63.462 | 1.00 | 26.26 | 6 |
| ATOM | 5709 | CD2 | LEU | A | 1026 | 91.529 | 52.247 | 63.086 | 1.00 | 26.84 | 6 |
| ATOM | 5710 | C | LEU | A | 1026 | 94.009 | 51.002 | 64.017 | 1.00 | 25.98 | 6 |
| ATOM | 5711 | O | LEU | A | 1026 | 94.302 | 52.200 | 64.127 | 1.00 | 26.06 | 8 |
| ATOM | 5712 | N | LEU | A | 1027 | 93.977 | 50.214 | 65.041 | 1.00 | 53.16 | 7 |
| ATOM | 5713 | CA | LEU | A | 1027 | 94.312 | 50.783 | 66.305 | 1.00 | 53.16 | 6 |
| ATOM | 5714 | CB | LEU | A | 1027 | 94.350 | 49.710 | 67.365 | 1.00 | 53.16 | 6 |
| ATOM | 5715 | CG | LEU | A | 1027 | 93.108 | 48.839 | 67.356 | 1.00 | 53.16 | 6 |
| ATOM | 5716 | CD1 | LEU | A | 1027 | 93.243 | 47.722 | 68.386 | 1.00 | 53.16 | 6 |
| ATOM | 5717 | CD2 | LEU | A | 1027 | 91.855 | 49.678 | 67.613 | 1.00 | 53.16 | 6 |
| ATOM | 5718 | C | LEU | A | 1027 | 95.622 | 51.557 | 66.244 | 1.00 | 53.16 | 6 |
| ATOM | 5719 | O | LEU | A | 1027 | 95.751 | 52.604 | 66.896 | 1.00 | 53.16 | 8 |
| ATOM | 5720 | N | ILE | A | 1028 | 96.599 | 51.060 | 65.476 | 1.00 | 53.16 | 7 |
| ATOM | 5721 | CA | ILE | A | 1028 | 97.888 | 51.710 | 65.374 | 1.00 | 53.16 | 6 |
| ATOM | 5722 | CB | ILE | A | 1028 | 98.922 | 50.775 | 64.801 | 1.00 | 53.16 | 6 |
| ATOM | 5723 | CG2 | ILE | A | 1028 | 100.266 | 51.470 | 64.633 | 1.00 | 53.16 | 6 |
| ATOM | 5724 | CG1 | ILE | A | 1028 | 99.063 | 49.577 | 65.733 | 1.00 | 53.16 | 6 |
| ATOM | 5725 | CD1 | ILE | A | 1028 | 99.902 | 48.453 | 65.160 | 1.00 | 53.16 | 6 |
| ATOM | 5726 | C | ILE | A | 1028 | 97.786 | 52.960 | 64.543 | 1.00 | 53.16 | 6 |
| ATOM | 5727 | O | ILE | A | 1028 | 98.525 | 53.923 | 64.745 | 1.00 | 53.16 | 8 |
| ATOM | 5728 | N | ILE | A | 1029 | 96.861 | 52.952 | 63.602 | 1.00 | 53.16 | 7 |
| ATOM | 5729 | CA | ILE | A | 1029 | 96.664 | 54.111 | 62.756 | 1.00 | 53.16 | 6 |
| ATOM | 5730 | CB | ILE | A | 1029 | 95.787 | 53.750 | 61.532 | 1.00 | 53.16 | 6 |
| ATOM | 5731 | CG2 | ILE | A | 1029 | 94.401 | 53.326 | 61.987 | 1.00 | 53.16 | 6 |
| ATOM | 5732 | CG1 | ILE | A | 1029 | 95.723 | 54.920 | 60.552 | 1.00 | 53.16 | 6 |
| ATOM | 5733 | CD1 | ILE | A | 1029 | 94.969 | 56.124 | 61.078 | 1.00 | 53.16 | 6 |
| ATOM | 5734 | C | ILE | A | 1029 | 95.991 | 55.179 | 63.624 | 1.00 | 53.16 | 6 |
| ATOM | 5735 | O | ILE | A | 1029 | 96.372 | 56.347 | 63.588 | 1.00 | 53.16 | 8 |
| ATOM | 5736 | N | LEU | A | 1030 | 95.008 | 54.758 | 64.418 | 1.00 | 53.16 | 7 |
| ATOM | 5737 | CA | LEU | A | 1030 | 94.302 | 55.664 | 65.332 | 1.00 | 53.16 | 6 |
| ATOM | 5738 | CB | LEU | A | 1030 | 93.198 | 54.928 | 66.117 | 1.00 | 53.16 | 6 |
| ATOM | 5739 | CG | LEU | A | 1030 | 92.003 | 54.323 | 65.368 | 1.00 | 53.16 | 6 |
| ATOM | 5740 | CD1 | LEU | A | 1030 | 91.075 | 53.597 | 66.339 | 1.00 | 53.16 | 6 |
| ATOM | 5741 | CD2 | LEU | A | 1030 | 91.249 | 55.407 | 64.622 | 1.00 | 53.16 | 6 |
| ATOM | 5742 | C | LEU | A | 1030 | 95.246 | 56.309 | 66.365 | 1.00 | 53.16 | 6 |
| ATOM | 5743 | O | LEU | A | 1030 | 95.169 | 57.511 | 66.659 | 1.00 | 53.16 | 8 |
| ATOM | 5744 | N | PHE | A | 1031 | 96.111 | 55.477 | 66.903 | 1.00 | 53.16 | 7 |
| ATOM | 5745 | CA | PHE | A | 1031 | 97.061 | 55.815 | 67.930 | 1.00 | 53.16 | 6 |
| ATOM | 5746 | CB | PHE | A | 1031 | 97.969 | 54.647 | 68.229 | 1.00 | 53.16 | 6 |
| ATOM | 5747 | CG | PHE | A | 1031 | 97.767 | 54.390 | 69.629 | 1.00 | 53.16 | 6 |
| ATOM | 5748 | CD1 | PHE | A | 1031 | 96.628 | 53.685 | 69.994 | 1.00 | 53.16 | 6 |
| ATOM | 5749 | CD2 | PHE | A | 1031 | 98.625 | 54.888 | 70.595 | 1.00 | 53.16 | 6 |
| ATOM | 5750 | CE1 | PHE | A | 1031 | 96.334 | 53.493 | 71.328 | 1.00 | 53.16 | 6 |
| ATOM | 5751 | CE2 | PHE | A | 1031 | 98.315 | 54.705 | 71.932 | 1.00 | 53.16 | 6 |
| ATOM | 5752 | CZ | PHE | A | 1031 | 97.166 | 54.002 | 72.287 | 1.00 | 53.16 | 6 |
| ATOM | 5753 | C | PHE | A | 1031 | 98.016 | 56.894 | 67.553 | 1.00 | 53.16 | 6 |
| ATOM | 5754 | O | PHE | A | 1031 | 97.958 | 58.062 | 67.934 | 1.00 | 53.16 | 8 |
| ATOM | 5755 | N | SER | A | 1032 | 98.897 | 56.400 | 66.763 | 1.00 | 53.16 | 7 |
| ATOM | 5756 | CA | SER | A | 1032 | 99.906 | 57.174 | 66.204 | 1.00 | 53.16 | 6 |
| ATOM | 5757 | CB | SER | A | 1032 | 100.192 | 56.681 | 64.774 | 1.00 | 53.16 | 6 |
| ATOM | 5758 | OG | SER | A | 1032 | 99.549 | 55.440 | 64.525 | 1.00 | 53.16 | 8 |
| ATOM | 5759 | C | SER | A | 1032 | 99.470 | 58.620 | 66.224 | 1.00 | 53.16 | 6 |
| ATOM | 5760 | O | SER | A | 1032 | 99.985 | 59.427 | 66.999 | 1.00 | 53.16 | 8 |
| ATOM | 5761 | N | MET | A | 1033 | 98.511 | 58.949 | 65.367 | 1.00 | 53.16 | 7 |
| ATOM | 5762 | CA | MET | A | 1033 | 98.032 | 60.309 | 65.333 | 1.00 | 53.16 | 6 |
| ATOM | 5763 | CB | MET | A | 1033 | 96.663 | 60.404 | 64.653 | 1.00 | 53.16 | 6 |
| ATOM | 5764 | CG | MET | A | 1033 | 96.604 | 59.757 | 63.294 | 1.00 | 53.16 | 6 |
| ATOM | 5765 | SD | MET | A | 1033 | 94.970 | 59.901 | 62.550 | 1.00 | 53.16 | 16 |
| ATOM | 5766 | CE | MET | A | 1033 | 95.043 | 61.552 | 61.856 | 1.00 | 53.16 | 6 |
| ATOM | 5767 | C | MET | A | 1033 | 97.936 | 60.876 | 66.764 | 1.00 | 53.16 | 6 |
| ATOM | 5768 | O | MET | A | 1033 | 98.949 | 61.264 | 67.355 | 1.00 | 53.16 | 8 |
| ATOM | 5769 | N | MET | A | 1034 | 96.745 | 60.929 | 67.304 | 1.00 | 53.16 | 7 |
| ATOM | 5770 | CA | MET | A | 1034 | 96.522 | 61.530 | 68.624 | 1.00 | 53.16 | 6 |
| ATOM | 5771 | CB | MET | A | 1034 | 95.424 | 60.788 | 69.370 | 1.00 | 208.01 | 6 |
| ATOM | 5772 | CG | MET | A | 1034 | 94.584 | 61.736 | 70.202 | 1.00 | 208.01 | 6 |
| ATOM | 5773 | SD | MET | A | 1034 | 93.301 | 60.886 | 71.138 | 1.00 | 208.01 | 16 |
| ATOM | 5774 | CE | MET | A | 1034 | 94.289 | 60.015 | 72.351 | 1.00 | 208.01 | 6 |
| ATOM | 5775 | C | MET | A | 1034 | 97.751 | 61.715 | 69.499 | 1.00 | 53.16 | 6 |
| ATOM | 5776 | O | MET | A | 1034 | 97.931 | 62.803 | 70.046 | 1.00 | 53.16 | 8 |
| ATOM | 5777 | N | LEU | A | 1035 | 98.567 | 60.730 | 69.658 | 1.00 | 70.80 | 7 |
| ATOM | 5778 | CA | LEU | A | 1035 | 99.700 | 61.018 | 70.488 | 1.00 | 70.80 | 6 |
| ATOM | 5779 | CB | LEU | A | 1035 | 100.512 | 59.778 | 70.781 | 1.00 | 125.69 | 6 |
| ATOM | 5780 | CG | LEU | A | 1035 | 100.364 | 59.269 | 72.196 | 1.00 | 125.69 | 6 |
| ATOM | 5781 | CD1 | LEU | A | 1035 | 98.939 | 59.402 | 72.673 | 1.00 | 125.69 | 6 |
| ATOM | 5782 | CD2 | LEU | A | 1035 | 100.835 | 57.822 | 72.299 | 1.00 | 125.69 | 6 |
| ATOM | 5783 | C | LEU | A | 1035 | 100.562 | 62.067 | 69.807 | 1.00 | 70.80 | 6 |
| ATOM | 5784 | O | LEU | A | 1035 | 100.678 | 63.178 | 70.334 | 1.00 | 70.80 | 8 |
| ATOM | 5785 | N | MET | A | 1036 | 101.161 | 61.762 | 68.688 | 1.00 | 114.89 | 7 |
| ATOM | 5786 | CA | MET | A | 1036 | 101.926 | 62.857 | 68.185 | 1.00 | 114.89 | 6 |
| ATOM | 5787 | CB | MET | A | 1036 | 101.701 | 63.110 | 66.691 | 1.00 | 164.85 | 6 |
| ATOM | 5788 | CG | MET | A | 1036 | 102.415 | 62.138 | 65.765 | 1.00 | 164.85 | 6 |
| ATOM | 5789 | SD | MET | A | 1036 | 103.999 | 61.600 | 66.420 | 1.00 | 170.26 | 16 |
| ATOM | 5790 | CE | MET | A | 1036 | 104.607 | 60.618 | 65.055 | 1.00 | 168.19 | 6 |
| ATOM | 5791 | C | MET | A | 1036 | 101.426 | 64.057 | 69.043 | 1.00 | 114.89 | 6 |
| ATOM | 5792 | O | MET | A | 1036 | 102.132 | 65.040 | 69.246 | 1.00 | 114.89 | 8 |
| ATOM | 5793 | N | THR | A | 1037 | 100.172 | 63.871 | 69.564 | 1.00 | 171.76 | 7 |
| ATOM | 5794 | CA | THR | A | 1037 | 99.274 | 64.858 | 70.290 | 1.00 | 171.76 | 6 |
| ATOM | 5795 | CB | THR | A | 1037 | 97.828 | 64.739 | 69.788 | 1.00 | 189.82 | 6 |
| ATOM | 5796 | OG1 | THR | A | 1037 | 97.377 | 66.001 | 69.267 | 1.00 | 189.82 | 8 |
| ATOM | 5797 | CG2 | THR | A | 1037 | 96.911 | 64.308 | 70.923 | 1.00 | 189.82 | 6 |
| ATOM | 5798 | C | THR | A | 1037 | 98.899 | 64.663 | 71.762 | 1.00 | 171.76 | 6 |
| ATOM | 5799 | O | THR | A | 1037 | 97.815 | 65.122 | 72.129 | 1.00 | 171.76 | 8 |
| ATOM | 5800 | N | GLY | A | 1038 | 99.635 | 64.047 | 72.666 | 1.00 | 192.87 | 7 |
| ATOM | 5801 | CA | GLY | A | 1038 | 98.987 | 63.757 | 73.978 | 1.00 | 198.17 | 6 |
| ATOM | 5802 | C | GLY | A | 1038 | 99.388 | 64.530 | 75.239 | 1.00 | 201.70 | 6 |
| ATOM | 5803 | O | GLY | A | 1038 | 98.555 | 64.928 | 76.039 | 1.00 | 201.70 | 8 |
| ATOM | 5804 | N | GLU | A | 1046 | 109.105 | 54.836 | 72.571 | 1.00 | 208.41 | 7 |
| ATOM | 5805 | CA | GLU | A | 1046 | 107.968 | 55.471 | 71.924 | 1.00 | 208.41 | 6 |
| ATOM | 5806 | CB | GLU | A | 1046 | 108.030 | 55.233 | 70.422 | 1.00 | 207.86 | 6 |
| ATOM | 5807 | CG | GLU | A | 1046 | 108.010 | 56.501 | 69.583 | 1.00 | 208.41 | 6 |
| ATOM | 5808 | CD | GLU | A | 1046 | 109.104 | 56.531 | 68.512 | 1.00 | 208.41 | 6 |
| ATOM | 5809 | OE1 | GLU | A | 1046 | 109.354 | 55.461 | 67.888 | 1.00 | 208.41 | 8 |
| ATOM | 5810 | OE2 | GLU | A | 1046 | 109.712 | 57.591 | 68.297 | 1.00 | 208.41 | 8 |
| ATOM | 5811 | C | GLU | A | 1046 | 106.642 | 54.975 | 72.510 | 1.00 | 208.41 | 6 |
| ATOM | 5812 | O | GLU | A | 1046 | 106.240 | 55.336 | 73.613 | 1.00 | 207.33 | 8 |
| ATOM | 5813 | N | ASP | A | 1047 | 106.100 | 53.943 | 71.727 | 1.00 | 133.52 | 7 |
| ATOM | 5814 | CA | ASP | A | 1047 | 104.928 | 53.072 | 72.011 | 1.00 | 132.40 | 6 |
| ATOM | 5815 | CB | ASP | A | 1047 | 104.612 | 53.113 | 73.498 | 1.00 | 172.55 | 6 |
| ATOM | 5816 | CG | ASP | A | 1047 | 105.448 | 52.034 | 74.159 | 1.00 | 174.95 | 6 |
| ATOM | 5817 | OD1 | ASP | A | 1047 | 104.942 | 50.896 | 74.272 | 1.00 | 176.37 | 8 |
| ATOM | 5818 | OD2 | ASP | A | 1047 | 106.600 | 52.336 | 74.552 | 1.00 | 176.61 | 8 |
| ATOM | 5819 | C | ASP | A | 1047 | 103.704 | 53.279 | 71.113 | 1.00 | 131.14 | 6 |
| ATOM | 5820 | O | ASP | A | 1047 | 102.864 | 54.147 | 71.305 | 1.00 | 131.06 | 8 |
| ATOM | 5821 | N | ILE | A | 1048 | 103.676 | 52.422 | 70.135 | 1.00 | 53.16 | 7 |
| ATOM | 5822 | CA | ILE | A | 1048 | 102.761 | 52.251 | 69.023 | 1.00 | 53.16 | 6 |
| ATOM | 5823 | CB | ILE | A | 1048 | 103.038 | 53.306 | 67.940 | 1.00 | 147.54 | 6 |
| ATOM | 5824 | CG2 | ILE | A | 1048 | 104.091 | 52.801 | 66.966 | 1.00 | 147.03 | 6 |
| ATOM | 5825 | CG1 | ILE | A | 1048 | 101.766 | 53.689 | 67.184 | 1.00 | 148.57 | 6 |
| ATOM | 5826 | CD1 | ILE | A | 1048 | 101.156 | 52.551 | 66.392 | 1.00 | 133.82 | 6 |
| ATOM | 5827 | C | ILE | A | 1048 | 103.248 | 50.896 | 68.631 | 1.00 | 53.16 | 6 |
| ATOM | 5828 | O | ILE | A | 1048 | 102.760 | 50.162 | 67.771 | 1.00 | 53.16 | 8 |
| ATOM | 5829 | N | GLU | A | 1049 | 104.304 | 50.718 | 69.437 | 1.00 | 59.93 | 7 |
| ATOM | 5830 | CA | GLU | A | 1049 | 105.135 | 49.568 | 69.556 | 1.00 | 59.43 | 6 |
| ATOM | 5831 | CB | GLU | A | 1049 | 106.630 | 49.919 | 69.670 | 1.00 | 167.62 | 6 |
| ATOM | 5832 | CG | GLU | A | 1049 | 107.447 | 49.023 | 68.738 | 1.00 | 172.10 | 6 |
| ATOM | 5833 | CD | GLU | A | 1049 | 108.832 | 49.558 | 68.373 | 1.00 | 175.65 | 6 |
| ATOM | 5834 | OE1 | GLU | A | 1049 | 108.928 | 50.490 | 67.545 | 1.00 | 177.55 | 8 |
| ATOM | 5835 | OE2 | GLU | A | 1049 | 109.813 | 49.052 | 68.936 | 1.00 | 178.24 | 8 |
| ATOM | 5836 | C | GLU | A | 1049 | 104.637 | 48.728 | 70.727 | 1.00 | 57.90 | 6 |
| ATOM | 5837 | O | GLU | A | 1049 | 104.608 | 47.510 | 70.586 | 1.00 | 59.47 | 8 |
| ATOM | 5838 | N | TYR | A | 1050 | 104.240 | 49.318 | 71.879 | 1.00 | 71.92 | 7 |
| ATOM | 5839 | CA | TYR | A | 1050 | 103.710 | 48.402 | 72.897 | 1.00 | 67.86 | 6 |
| ATOM | 5840 | CB | TYR | A | 1050 | 103.436 | 48.952 | 74.300 | 1.00 | 107.06 | 6 |
| ATOM | 5841 | CG | TYR | A | 1050 | 103.066 | 47.801 | 75.236 | 1.00 | 104.92 | 6 |
| ATOM | 5842 | CD1 | TYR | A | 1050 | 104.073 | 47.085 | 75.886 | 1.00 | 104.95 | 6 |
| ATOM | 5843 | CE1 | TYR | A | 1050 | 103.769 | 46.003 | 76.715 | 1.00 | 105.25 | 6 |
| ATOM | 5844 | CD2 | TYR | A | 1050 | 101.744 | 47.406 | 75.442 | 1.00 | 102.95 | 6 |
| ATOM | 5845 | CE2 | TYR | A | 1050 | 101.433 | 46.323 | 76.269 | 1.00 | 104.05 | 6 |
| ATOM | 5846 | CZ | TYR | A | 1050 | 102.452 | 45.625 | 76.900 | 1.00 | 104.33 | 6 |
| ATOM | 5847 | OH | TYR | A | 1050 | 102.163 | 44.544 | 77.704 | 1.00 | 104.43 | 8 |
| ATOM | 5848 | C | TYR | A | 1050 | 102.391 | 47.897 | 72.351 | 1.00 | 67.37 | 6 |
| ATOM | 5849 | O | TYR | A | 1050 | 101.981 | 46.791 | 72.675 | 1.00 | 64.41 | 8 |
| ATOM | 5850 | N | ILE | A | 1051 | 102.043 | 48.475 | 71.755 | 1.00 | 39.30 | 7 |
| ATOM | 5851 | CA | ILE | A | 1051 | 100.896 | 47.854 | 71.115 | 1.00 | 39.35 | 6 |
| ATOM | 5852 | CB | ILE | A | 1051 | 99.928 | 48.916 | 70.536 | 1.00 | 39.54 | 6 |
| ATOM | 5853 | CG2 | ILE | A | 1051 | 100.474 | 49.485 | 69.224 | 1.00 | 39.48 | 6 |
| ATOM | 5854 | CG1 | ILE | A | 1051 | 98.544 | 48.283 | 70.342 | 1.00 | 37.48 | 6 |
| ATOM | 5855 | CD1 | ILE | A | 1051 | 97.460 | 49.248 | 69.910 | 1.00 | 41.66 | 6 |
| ATOM | 5856 | C | ILE | A | 1051 | 101.258 | 46.842 | 70.031 | 1.00 | 40.18 | 6 |
| ATOM | 5857 | O | ILE | A | 1051 | 100.490 | 45.920 | 69.761 | 1.00 | 39.01 | 8 |
| ATOM | 5858 | N | ARG | A | 1052 | 102.417 | 47.036 | 69.409 | 1.00 | 40.41 | 7 |
| ATOM | 5859 | CA | ARG | A | 1052 | 102.869 | 46.078 | 68.411 | 1.00 | 41.90 | 6 |
| ATOM | 5860 | CB | ARG | A | 1052 | 104.123 | 46.586 | 67.703 | 1.00 | 46.30 | 6 |
| ATOM | 5861 | CG | ARG | A | 1052 | 103.941 | 47.900 | 66.967 | 1.00 | 51.38 | 6 |
| ATOM | 5862 | CD | ARG | A | 1052 | 105.244 | 48.346 | 66.327 | 1.00 | 54.14 | 6 |
| ATOM | 5863 | NE | ARG | A | 1052 | 105.199 | 48.264 | 64.872 | 1.00 | 56.54 | 7 |
| ATOM | 5864 | CZ | ARG | A | 1052 | 106.243 | 48.487 | 64.083 | 1.00 | 58.76 | 6 |
| ATOM | 5865 | NH1 | ARG | A | 1052 | 107.416 | 48.806 | 64.612 | 1.00 | 57.11 | 7 |
| ATOM | 5866 | NH2 | ARG | A | 1052 | 106.113 | 48.389 | 62.767 | 1.00 | 58.91 | 7 |
| ATOM | 5867 | C | ARG | A | 1052 | 103.154 | 44.718 | 69.030 | 1.00 | 40.79 | 6 |
| ATOM | 5868 | O | ARG | A | 1052 | 102.710 | 43.698 | 68.557 | 1.00 | 39.07 | 8 |
| ATOM | 5869 | N | ASP | A | 1053 | 103.853 | 44.765 | 70.176 | 1.00 | 40.45 | 7 |
| ATOM | 5870 | CA | ASP | A | 1053 | 104.144 | 43.533 | 70.902 | 1.00 | 40.24 | 6 |
| ATOM | 5871 | CB | ASP | A | 1053 | 105.175 | 43.769 | 72.017 | 1.00 | 43.92 | 6 |
| ATOM | 5872 | CG | ASP | A | 1053 | 106.528 | 44.232 | 71.493 | 1.00 | 47.20 | 6 |
| ATOM | 5873 | OD1 | ASP | A | 1053 | 106.894 | 43.874 | 70.352 | 1.00 | 48.44 | 8 |
| ATOM | 5874 | OD2 | ASP | A | 1053 | 107.240 | 44.943 | 72.240 | 1.00 | 49.28 | 8 |
| ATOM | 5875 | C | ASP | A | 1053 | 102.860 | 42.980 | 71.526 | 1.00 | 39.20 | 6 |
| ATOM | 5876 | O | ASP | A | 1053 | 102.619 | 41.772 | 71.489 | 1.00 | 38.81 | 8 |
| ATOM | 5877 | N | ALA | A | 1054 | 102.036 | 43.865 | 72.093 | 1.00 | 35.67 | 7 |
| ATOM | 5878 | CA | ALA | A | 1054 | 100.787 | 43.448 | 72.727 | 1.00 | 34.45 | 6 |
| ATOM | 5879 | CB | ALA | A | 1054 | 100.058 | 44.654 | 73.323 | 1.00 | 35.08 | 6 |
| ATOM | 5880 | C | ALA | A | 1054 | 99.880 | 42.730 | 71.742 | 1.00 | 33.22 | 6 |
| ATOM | 5881 | O | ALA | A | 1054 | 99.153 | 41.822 | 72.113 | 1.00 | 32.20 | 8 |
| ATOM | 5882 | N | LEU | A | 1055 | 99.922 | 43.150 | 70.486 | 1.00 | 33.58 | 7 |
| ATOM | 5883 | CA | LEU | A | 1055 | 99.104 | 42.524 | 69.450 | 1.00 | 34.78 | 6 |
| ATOM | 5884 | CB | LEU | A | 1055 | 98.697 | 43.559 | 68.410 | 1.00 | 31.17 | 6 |
| ATOM | 5885 | CG | LEU | A | 1055 | 97.637 | 44.535 | 68.926 | 1.00 | 29.73 | 6 |
| ATOM | 5886 | CD1 | LEU | A | 1055 | 97.535 | 45.707 | 67.982 | 1.00 | 26.72 | 6 |
| ATOM | 5887 | CD2 | LEU | A | 1055 | 96.291 | 43.813 | 69.081 | 1.00 | 26.17 | 6 |
| ATOM | 5888 | C | LEU | A | 1055 | 99.874 | 41.390 | 68.789 | 1.00 | 35.64 | 6 |
| ATOM | 5889 | O | LEU | A | 1055 | 99.476 | 40.882 | 67.740 | 1.00 | 35.02 | 8 |
| ATOM | 5890 | N | THR | A | 1056 | 100.968 | 40.990 | 69.430 | 1.00 | 38.51 | 7 |
| ATOM | 5891 | CA | THR | A | 1056 | 101.822 | 39.918 | 68.931 | 1.00 | 42.12 | 6 |
| ATOM | 5892 | CB | THR | A | 1056 | 101.317 | 38.533 | 69.423 | 1.00 | 42.48 | 6 |
| ATOM | 5893 | OG1 | THR | A | 1056 | 102.105 | 37.499 | 68.823 | 1.00 | 49.40 | 8 |
| ATOM | 5894 | CG2 | THR | A | 1056 | 99.861 | 38.320 | 69.061 | 1.00 | 43.09 | 6 |
| ATOM | 5895 | C | THR | A | 1056 | 101.898 | 39.951 | 67.400 | 1.00 | 42.96 | 6 |
| ATOM | 5896 | O | THR | A | 1056 | 101.614 | 38.959 | 66.721 | 1.00 | 42.73 | 8 |
| ATOM | 5897 | N | VAL | A | 1057 | 102.279 | 41.112 | 66.871 | 1.00 | 42.66 | 7 |
| ATOM | 5898 | CA | VAL | A | 1057 | 102.398 | 41.316 | 65.433 | 1.00 | 44.19 | 6 |
| ATOM | 5899 | CB | VAL | A | 1057 | 102.828 | 42.764 | 65.114 | 1.00 | 44.89 | 6 |
| ATOM | 5900 | CG1 | VAL | A | 1057 | 103.118 | 42.910 | 63.631 | 1.00 | 44.47 | 6 |
| ATOM | 5901 | CG2 | VAL | A | 1057 | 101.730 | 43.734 | 65.528 | 1.00 | 44.12 | 6 |
| ATOM | 5902 | C | VAL | A | 1057 | 103.395 | 40.366 | 64.784 | 1.00 | 45.84 | 6 |
| ATOM | 5903 | O | VAL | A | 1057 | 104.517 | 40.200 | 65.265 | 1.00 | 45.98 | 8 |
| ATOM | 5904 | N | GLY | A | 1058 | 102.975 | 39.742 | 63.690 | 1.00 | 46.50 | 7 |
| ATOM | 5905 | CA | GLY | A | 1058 | 103.849 | 38.829 | 62.984 | 1.00 | 49.30 | 6 |
| ATOM | 5906 | C | GLY | A | 1058 | 103.562 | 37.358 | 63.202 | 1.00 | 50.55 | 6 |
| ATOM | 5907 | O | GLY | A | 1058 | 103.760 | 36.550 | 62.299 | 1.00 | 51.66 | 8 |
| ATOM | 5908 | N | LYS | A | 1059 | 103.067 | 36.989 | 64.385 | 1.00 | 50.32 | 7 |
| ATOM | 5909 | CA | LYS | A | 1059 | 102.819 | 35.596 | 64.713 | 1.00 | 49.64 | 6 |
| ATOM | 5910 | CB | LYS | A | 1059 | 102.904 | 35.388 | 66.224 | 1.00 | 51.29 | 6 |
| ATOM | 5911 | CG | LYS | A | 1059 | 104.265 | 35.724 | 66.814 | 1.00 | 53.09 | 6 |
| ATOM | 5912 | CD | LYS | A | 1059 | 104.362 | 35.288 | 68.265 | 1.00 | 56.35 | 6 |
| ATOM | 5913 | CE | LYS | A | 1059 | 105.721 | 35.632 | 68.854 | 1.00 | 58.08 | 6 |
| ATOM | 5914 | NZ | LYS | A | 1059 | 105.835 | 35.205 | 70.275 | 1.00 | 59.17 | 7 |
| ATOM | 5915 | C | LYS | A | 1059 | 101.470 | 35.146 | 64.170 | 1.00 | 48.94 | 6 |
| ATOM | 5916 | O | LYS | A | 1059 | 100.549 | 35.916 | 63.941 | 1.00 | 48.33 | 8 |
| ATOM | 5917 | N | ASN | A | 1060 | 101.344 | 33.841 | 63.954 | 1.00 | 48.46 | 7 |
| ATOM | 5918 | CA | ASN | A | 1060 | 100.109 | 33.218 | 63.479 | 1.00 | 47.99 | 6 |
| ATOM | 5919 | CB | ASN | A | 1060 | 100.392 | 31.812 | 62.940 | 1.00 | 49.47 | 6 |
| ATOM | 5920 | CG | ASN | A | 1060 | 100.563 | 30.798 | 64.045 | 1.00 | 49.99 | 6 |
| ATOM | 5921 | OD1 | ASN | A | 1060 | 101.569 | 30.822 | 64.752 | 1.00 | 51.12 | 8 |
| ATOM | 5922 | ND2 | ASN | A | 1060 | 99.593 | 29.908 | 64.210 | 1.00 | 52.46 | 7 |
| ATOM | 5923 | C | ASN | A | 1060 | 99.079 | 33.148 | 64.594 | 1.00 | 47.02 | 6 |
| ATOM | 5924 | O | ASN | A | 1060 | 99.366 | 33.419 | 65.762 | 1.00 | 46.58 | 8 |
| ATOM | 5925 | N | GLU | A | 1061 | 97.880 | 32.769 | 64.237 | 1.00 | 46.13 | 7 |
| ATOM | 5926 | CA | GLU | A | 1061 | 96.785 | 32.765 | 65.194 | 1.00 | 47.21 | 6 |
| ATOM | 5927 | CB | GLU | A | 1061 | 95.455 | 32.554 | 64.475 | 1.00 | 47.08 | 6 |
| ATOM | 5928 | CG | GLU | A | 1061 | 94.919 | 33.846 | 63.869 | 1.00 | 49.64 | 6 |
| ATOM | 5929 | CD | GLU | A | 1061 | 93.430 | 33.833 | 63.598 | 1.00 | 51.35 | 6 |
| ATOM | 5930 | OE1 | GLU | A | 1061 | 92.884 | 34.911 | 63.315 | 1.00 | 53.04 | 8 |
| ATOM | 5931 | OE2 | GLU | A | 1061 | 92.805 | 32.753 | 63.666 | 1.00 | 53.48 | 8 |
| ATOM | 5932 | C | GLU | A | 1061 | 96.995 | 31.827 | 66.360 | 1.00 | 46.86 | 6 |
| ATOM | 5933 | O | GLU | A | 1061 | 96.974 | 32.287 | 67.489 | 1.00 | 46.22 | 8 |
| ATOM | 5934 | N | GLU | A | 1062 | 97.206 | 30.565 | 66.174 | 1.00 | 46.82 | 7 |
| ATOM | 5935 | CA | GLU | A | 1062 | 97.395 | 29.728 | 67.339 | 1.00 | 47.47 | 6 |
| ATOM | 5936 | CB | GLU | A | 1062 | 97.736 | 28.296 | 66.933 | 1.00 | 49.16 | 6 |
| ATOM | 5937 | CG | GLU | A | 1062 | 96.553 | 27.320 | 66.937 | 1.00 | 51.24 | 6 |
| ATOM | 5938 | CD | GLU | A | 1062 | 95.660 | 27.339 | 68.165 | 1.00 | 52.23 | 6 |
| ATOM | 5939 | OE1 | GLU | A | 1062 | 96.183 | 27.320 | 69.299 | 1.00 | 53.22 | 8 |
| ATOM | 5940 | OE2 | GLU | A | 1062 | 94.425 | 27.365 | 67.990 | 1.00 | 54.15 | 8 |
| ATOM | 5941 | C | GLU | A | 1062 | 98.477 | 30.278 | 68.315 | 1.00 | 46.24 | 6 |
| ATOM | 5942 | O | GLU | A | 1062 | 98.206 | 30.324 | 69.520 | 1.00 | 44.89 | 8 |
| ATOM | 5943 | N | ASP | A | 1063 | 99.673 | 30.681 | 67.856 | 1.00 | 46.08 | 7 |
| ATOM | 5944 | CA | ASP | A | 1063 | 100.685 | 31.255 | 68.771 | 1.00 | 47.05 | 6 |
| ATOM | 5945 | CB | ASP | A | 1063 | 101.978 | 31.649 | 68.044 | 1.00 | 49.11 | 6 |
| ATOM | 5946 | CG | ASP | A | 1063 | 102.831 | 30.439 | 67.679 | 1.00 | 52.57 | 6 |
| ATOM | 5947 | OD1 | ASP | A | 1063 | 102.989 | 29.542 | 68.530 | 1.00 | 53.45 | 8 |
| ATOM | 5948 | OD2 | ASP | A | 1063 | 103.328 | 30.388 | 66.531 | 1.00 | 52.95 | 8 |
| ATOM | 5949 | C | ASP | A | 1063 | 100.105 | 32.471 | 69.474 | 1.00 | 45.56 | 6 |
| ATOM | 5950 | O | ASP | A | 1063 | 100.449 | 32.748 | 70.631 | 1.00 | 45.06 | 8 |
| ATOM | 5951 | N | ALA | A | 1064 | 99.235 | 33.193 | 68.775 | 1.00 | 44.08 | 7 |
| ATOM | 5952 | CA | ALA | A | 1064 | 98.583 | 34.361 | 69.363 | 1.00 | 41.41 | 6 |
| ATOM | 5953 | CB | ALA | A | 1064 | 97.722 | 35.064 | 68.321 | 1.00 | 39.65 | 6 |
| ATOM | 5954 | C | ALA | A | 1064 | 97.719 | 33.860 | 70.520 | 1.00 | 39.90 | 6 |
| ATOM | 5955 | O | ALA | A | 1064 | 97.852 | 34.310 | 71.663 | 1.00 | 37.36 | 8 |
| ATOM | 5956 | N | LYS | A | 1065 | 96.836 | 32.915 | 70.208 | 1.00 | 39.14 | 7 |
| ATOM | 5957 | CA | LYS | A | 1065 | 95.959 | 32.320 | 71.205 | 1.00 | 38.66 | 6 |
| ATOM | 5958 | CB | LYS | A | 1065 | 95.228 | 31.121 | 70.598 | 1.00 | 38.31 | 6 |
| ATOM | 5959 | CG | LYS | A | 1065 | 94.507 | 30.240 | 71.600 | 1.00 | 36.74 | 6 |
| ATOM | 5960 | CD | LYS | A | 1065 | 93.660 | 29.189 | 70.897 | 1.00 | 37.98 | 6 |
| ATOM | 5961 | CE | LYS | A | 1065 | 93.042 | 28.224 | 71.903 | 1.00 | 38.37 | 6 |
| ATOM | 5962 | NZ | LYS | A | 1065 | 92.053 | 27.296 | 71.279 | 1.00 | 38.40 | 7 |
| ATOM | 5963 | C | LYS | A | 1065 | 96.806 | 31.876 | 72.391 | 1.00 | 39.41 | 6 |
| ATOM | 5964 | O | LYS | A | 1065 | 96.509 | 32.197 | 73.544 | 1.00 | 38.65 | 8 |
| ATOM | 5965 | N | LYS | A | 1066 | 97.868 | 31.138 | 72.092 | 1.00 | 39.20 | 7 |
| ATOM | 5966 | CA | LYS | A | 1066 | 98.782 | 30.645 | 73.112 | 1.00 | 39.98 | 6 |
| ATOM | 5967 | CB | LYS | A | 1066 | 99.958 | 29.930 | 72.440 | 1.00 | 41.32 | 6 |
| ATOM | 5968 | CG | LYS | A | 1066 | 101.177 | 29.741 | 73.327 | 1.00 | 45.57 | 6 |
| ATOM | 5969 | CD | LYS | A | 1066 | 100.859 | 28.893 | 74.543 | 1.00 | 48.70 | 6 |
| ATOM | 5970 | CE | LYS | A | 1066 | 102.119 | 28.574 | 75.336 | 1.00 | 50.40 | 6 |
| ATOM | 5971 | NZ | LYS | A | 1066 | 101.790 | 27.827 | 76.579 | 1.00 | 51.30 | 7 |
| ATOM | 5972 | C | LYS | A | 1066 | 99.309 | 31.790 | 73.973 | 1.00 | 39.09 | 6 |
| ATOM | 5973 | O | LYS | A | 1066 | 99.337 | 31.701 | 75.199 | 1.00 | 39.69 | 8 |
| ATOM | 5974 | N | TYR | A | 1067 | 99.733 | 32.862 | 73.317 | 1.00 | 38.17 | 7 |
| ATOM | 5975 | CA | TYR | A | 1067 | 100.274 | 34.020 | 74.006 | 1.00 | 37.69 | 6 |
| ATOM | 5976 | CB | TYR | A | 1067 | 100.676 | 35.089 | 72.985 | 1.00 | 38.53 | 6 |
| ATOM | 5977 | CG | TYR | A | 1067 | 101.196 | 36.375 | 73.591 | 1.00 | 38.05 | 6 |
| ATOM | 5978 | CD1 | TYR | A | 1067 | 100.370 | 37.498 | 73.712 | 1.00 | 38.56 | 6 |
| ATOM | 5979 | CE1 | TYR | A | 1067 | 100.848 | 38.689 | 74.266 | 1.00 | 37.98 | 6 |
| ATOM | 5980 | CD2 | TYR | A | 1067 | 102.511 | 36.471 | 74.043 | 1.00 | 36.98 | 6 |
| ATOM | 5981 | CE2 | TYR | A | 1067 | 102.996 | 37.653 | 74.599 | 1.00 | 38.27 | 6 |
| ATOM | 5982 | CZ | TYR | A | 1067 | 102.161 | 38.757 | 74.706 | 1.00 | 38.75 | 6 |
| ATOM | 5983 | OH | TYR | A | 1067 | 102.650 | 39.927 | 75.246 | 1.00 | 39.46 | 8 |
| ATOM | 5984 | C | TYR | A | 1067 | 99.306 | 34.603 | 75.023 | 1.00 | 35.64 | 6 |
| ATOM | 5985 | O | TYR | A | 1067 | 99.685 | 34.847 | 76.166 | 1.00 | 35.55 | 8 |
| ATOM | 5986 | N | PHE | A | 1068 | 98.058 | 34.818 | 74.623 | 1.00 | 36.57 | 7 |
| ATOM | 5987 | CA | PHE | A | 1068 | 97.087 | 35.396 | 75.550 | 1.00 | 36.00 | 6 |
| ATOM | 5988 | CB | PHE | A | 1068 | 95.788 | 35.780 | 74.839 | 1.00 | 36.07 | 6 |
| ATOM | 5989 | CG | PHE | A | 1068 | 94.953 | 36.746 | 75.629 | 1.00 | 33.51 | 6 |
| ATOM | 5990 | CD1 | PHE | A | 1068 | 95.133 | 38.120 | 75.481 | 1.00 | 33.84 | 6 |
| ATOM | 5991 | CD2 | PHE | A | 1068 | 94.075 | 36.287 | 76.607 | 1.00 | 31.73 | 6 |
| ATOM | 5992 | CE1 | PHE | A | 1068 | 94.456 | 39.025 | 76.307 | 1.00 | 34.00 | 6 |
| ATOM | 5993 | CE2 | PHE | A | 1068 | 93.393 | 37.179 | 77.439 | 1.00 | 32.54 | 6 |
| ATOM | 5994 | CZ | PHE | A | 1068 | 93.587 | 38.553 | 77.290 | 1.00 | 34.18 | 6 |
| ATOM | 5995 | C | PHE | A | 1068 | 96.756 | 34.458 | 76.704 | 1.00 | 36.27 | 6 |
| ATOM | 5996 | O | PHE | A | 1068 | 96.627 | 34.892 | 77.849 | 1.00 | 36.86 | 8 |
| ATOM | 5997 | N | LEU | A | 1069 | 96.601 | 33.173 | 76.408 | 1.00 | 36.86 | 7 |
| ATOM | 5998 | CA | LEU | A | 1069 | 96.291 | 32.205 | 77.455 | 1.00 | 37.68 | 6 |
| ATOM | 5999 | CB | LEU | A | 1069 | 96.167 | 30.802 | 76.859 | 1.00 | 36.27 | 6 |
| ATOM | 6000 | CG | LEU | A | 1069 | 94.889 | 30.661 | 76.032 | 1.00 | 37.02 | 6 |
| ATOM | 6001 | CD1 | LEU | A | 1069 | 94.866 | 29.341 | 75.282 | 1.00 | 35.37 | 6 |
| ATOM | 6002 | CD2 | LEU | A | 1069 | 93.694 | 30.774 | 76.974 | 1.00 | 36.72 | 6 |
| ATOM | 6003 | C | LEU | A | 1069 | 97.368 | 32.247 | 78.534 | 1.00 | 37.68 | 6 |
| ATOM | 6004 | O | LEU | A | 1069 | 97.071 | 32.129 | 79.719 | 1.00 | 39.40 | 8 |
| ATOM | 6005 | N | ASP | A | 1070 | 98.617 | 32.442 | 78.123 | 1.00 | 38.74 | 7 |
| ATOM | 6006 | CA | ASP | A | 1070 | 99.718 | 32.518 | 79.070 | 1.00 | 39.24 | 6 |
| ATOM | 6007 | CB | ASP | A | 1070 | 101.063 | 32.481 | 78.333 | 1.00 | 40.04 | 6 |
| ATOM | 6008 | CG | ASP | A | 1070 | 101.339 | 31.128 | 77.696 | 1.00 | 44.75 | 6 |
| ATOM | 6009 | OD1 | ASP | A | 1070 | 102.462 | 30.918 | 77.193 | 1.00 | 46.70 | 8 |
| ATOM | 6010 | OD2 | ASP | A | 1070 | 100.434 | 30.268 | 77.697 | 1.00 | 45.89 | 8 |
| ATOM | 6011 | C | ASP | A | 1070 | 99.611 | 33.796 | 79.908 | 1.00 | 39.89 | 6 |
| ATOM | 6012 | O | ASP | A | 1070 | 100.014 | 33.818 | 81.067 | 1.00 | 39.66 | 8 |
| ATOM | 6013 | N | GLN | A | 1071 | 99.074 | 34.865 | 79.325 | 1.00 | 39.00 | 7 |
| ATOM | 6014 | CA | GLN | A | 1071 | 98.926 | 36.105 | 80.079 | 1.00 | 37.93 | 6 |
| ATOM | 6015 | CB | GLN | A | 1071 | 98.421 | 37.241 | 79.179 | 1.00 | 37.55 | 6 |
| ATOM | 6016 | CG | GLN | A | 1071 | 99.372 | 37.645 | 78.058 | 1.00 | 37.23 | 6 |
| ATOM | 6017 | CD | GLN | A | 1071 | 100.789 | 37.895 | 78.549 | 1.00 | 41.71 | 6 |
| ATOM | 6018 | OE1 | GLN | A | 1071 | 101.003 | 38.525 | 79.587 | 1.00 | 41.81 | 8 |
| ATOM | 6019 | NE2 | GLN | A | 1071 | 101.769 | 37.404 | 77.798 | 1.00 | 44.19 | 7 |
| ATOM | 6020 | C | GLN | A | 1071 | 97.929 | 35.844 | 81.209 | 1.00 | 36.56 | 6 |
| ATOM | 6021 | O | GLN | A | 1071 | 98.092 | 36.338 | 82.326 | 1.00 | 37.36 | 8 |
| ATOM | 6022 | N | ILE | A | 1072 | 96.905 | 35.052 | 80.912 | 1.00 | 36.58 | 7 |
| ATOM | 6023 | CA | ILE | A | 1072 | 95.887 | 34.707 | 81.897 | 1.00 | 38.85 | 6 |
| ATOM | 6024 | CB | ILE | A | 1072 | 94.768 | 33.832 | 81.262 | 1.00 | 38.98 | 6 |
| ATOM | 6025 | CG2 | ILE | A | 1072 | 93.917 | 33.185 | 82.350 | 1.00 | 36.69 | 6 |
| ATOM | 6026 | CG1 | ILE | A | 1072 | 93.903 | 34.691 | 80.332 | 1.00 | 39.19 | 6 |
| ATOM | 6027 | CD1 | ILE | A | 1072 | 92.802 | 33.924 | 79.619 | 1.00 | 38.94 | 6 |
| ATOM | 6028 | C | ILE | A | 1072 | 96.531 | 33.946 | 83.059 | 1.00 | 40.31 | 6 |
| ATOM | 6029 | O | ILE | A | 1072 | 96.137 | 34.102 | 84.220 | 1.00 | 40.30 | 8 |
| ATOM | 6030 | N | GLU | A | 1073 | 97.529 | 33.129 | 82.733 | 1.00 | 40.82 | 7 |
| ATOM | 6031 | CA | GLU | A | 1073 | 98.236 | 32.348 | 83.735 | 1.00 | 41.75 | 6 |
| ATOM | 6032 | CB | GLU | A | 1073 | 99.104 | 31.288 | 83.056 | 1.00 | 43.03 | 6 |
| ATOM | 6033 | CG | GLU | A | 1073 | 98.301 | 30.112 | 82.537 | 1.00 | 45.18 | 6 |
| ATOM | 6034 | CD | GLU | A | 1073 | 97.418 | 29.518 | 83.620 | 1.00 | 46.85 | 6 |
| ATOM | 6035 | OE1 | GLU | A | 1073 | 97.955 | 29.169 | 84.696 | 1.00 | 49.00 | 8 |
| ATOM | 6036 | OE2 | GLU | A | 1073 | 96.190 | 29.406 | 83.397 | 1.00 | 44.42 | 8 |
| ATOM | 6037 | C | GLU | A | 1073 | 99.093 | 33.222 | 84.641 | 1.00 | 41.37 | 6 |
| ATOM | 6038 | O | GLU | A | 1073 | 99.124 | 33.018 | 85.855 | 1.00 | 41.97 | 8 |
| ATOM | 6039 | N | VAL | A | 1074 | 99.790 | 34.191 | 84.059 | 1.00 | 40.60 | 7 |
| ATOM | 6040 | CA | VAL | A | 1074 | 100.621 | 35.079 | 84.856 | 1.00 | 40.80 | 6 |
| ATOM | 6041 | CB | VAL | A | 1074 | 101.249 | 36.191 | 83.995 | 1.00 | 42.91 | 6 |
| ATOM | 6042 | CG1 | VAL | A | 1074 | 102.158 | 37.087 | 84.819 | 1.00 | 42.32 | 6 |
| ATOM | 6043 | CG2 | VAL | A | 1074 | 102.029 | 35.576 | 82.840 | 1.00 | 43.22 | 6 |
| ATOM | 6044 | C | VAL | A | 1074 | 99.750 | 35.707 | 85.939 | 1.00 | 40.05 | 6 |
| ATOM | 6045 | O | VAL | A | 1074 | 100.199 | 35.921 | 87.062 | 1.00 | 40.30 | 8 |
| ATOM | 6046 | N | CYS | A | 1075 | 98.504 | 36.004 | 85.593 | 1.00 | 39.08 | 7 |
| ATOM | 6047 | CA | CYS | A | 1075 | 97.595 | 36.630 | 86.540 | 1.00 | 39.63 | 6 |
| ATOM | 6048 | CB | CYS | A | 1075 | 96.366 | 37.160 | 85.829 | 1.00 | 39.71 | 6 |
| ATOM | 6049 | SG | CYS | A | 1075 | 96.701 | 38.543 | 84.692 | 1.00 | 40.09 | 16 |
| ATOM | 6050 | C | CYS | A | 1075 | 97.143 | 35.661 | 87.649 | 1.00 | 41.23 | 6 |
| ATOM | 6051 | O | CYS | A | 1075 | 97.092 | 36.017 | 88.828 | 1.00 | 41.69 | 8 |
| ATOM | 6052 | N | ARG | A | 1076 | 96.829 | 34.406 | 87.234 | 1.00 | 41.04 | 7 |
| ATOM | 6053 | CA | ARG | A | 1076 | 96.351 | 33.280 | 88.048 | 1.00 | 42.95 | 6 |
| ATOM | 6054 | CB | ARG | A | 1076 | 95.974 | 32.103 | 87.165 | 1.00 | 43.58 | 6 |
| ATOM | 6055 | CG | ARG | A | 1076 | 94.727 | 32.300 | 86.329 | 1.00 | 46.19 | 6 |
| ATOM | 6056 | CD | ARG | A | 1076 | 93.715 | 31.236 | 86.645 | 1.00 | 47.98 | 6 |
| ATOM | 6057 | NE | ARG | A | 1076 | 94.170 | 29.903 | 86.243 | 1.00 | 51.87 | 7 |
| ATOM | 6058 | CZ | ARG | A | 1076 | 93.625 | 28.784 | 86.682 | 1.00 | 53.01 | 6 |
| ATOM | 6059 | NH1 | ARG | A | 1076 | 92.637 | 28.826 | 87.555 | 1.00 | 53.91 | 7 |
| ATOM | 6060 | NH2 | ARG | A | 1076 | 94.063 | 27.619 | 86.232 | 1.00 | 53.99 | 7 |
| ATOM | 6061 | C | ARG | A | 1076 | 97.396 | 32.778 | 88.975 | 1.00 | 41.88 | 6 |
| ATOM | 6062 | O | ARG | A | 1076 | 97.229 | 31.796 | 89.703 | 1.00 | 43.99 | 8 |
| ATOM | 6063 | N | ASP | A | 1077 | 98.498 | 33.489 | 88.945 | 1.00 | 42.14 | 7 |
| ATOM | 6064 | CA | ASP | A | 1077 | 99.616 | 33.108 | 89.728 | 1.00 | 42.73 | 6 |
| ATOM | 6065 | CB | ASP | A | 1077 | 100.622 | 32.431 | 88.830 | 1.00 | 45.06 | 6 |
| ATOM | 6066 | CG | ASP | A | 1077 | 99.860 | 31.489 | 87.909 | 1.00 | 49.08 | 6 |
| ATOM | 6067 | OD1 | ASP | A | 1077 | 99.047 | 30.699 | 88.440 | 1.00 | 49.67 | 8 |
| ATOM | 6068 | OD2 | ASP | A | 1077 | 100.067 | 31.530 | 86.677 | 1.00 | 51.30 | 8 |
| ATOM | 6069 | C | ASP | A | 1077 | 100.178 | 34.302 | 90.464 | 1.00 | 43.19 | 6 |
| ATOM | 6070 | O | ASP | A | 1077 | 101.027 | 34.149 | 91.354 | 1.00 | 44.32 | 8 |
| ATOM | 6071 | N | LYS | A | 1078 | 99.710 | 35.479 | 90.108 | 1.00 | 41.69 | 7 |
| ATOM | 6072 | CA | LYS | A | 1078 | 100.179 | 36.606 | 90.843 | 1.00 | 41.39 | 6 |
| ATOM | 6073 | CB | LYS | A | 1078 | 100.130 | 37.869 | 89.982 | 1.00 | 42.13 | 6 |
| ATOM | 6074 | CG | LYS | A | 1078 | 101.476 | 38.561 | 89.851 | 1.00 | 43.27 | 6 |
| ATOM | 6075 | CD | LYS | A | 1078 | 102.405 | 37.773 | 88.962 | 1.00 | 44.41 | 6 |
| ATOM | 6076 | CE | LYS | A | 1078 | 103.714 | 38.514 | 88.764 | 1.00 | 45.63 | 6 |
| ATOM | 6077 | NZ | LYS | A | 1078 | 104.653 | 37.754 | 87.896 | 1.00 | 47.98 | 7 |
| ATOM | 6078 | C | LYS | A | 1078 | 99.290 | 36.729 | 92.057 | 1.00 | 38.89 | 6 |
| ATOM | 6079 | O | LYS | A | 1078 | 99.676 | 37.266 | 93.083 | 1.00 | 38.69 | 8 |
| ATOM | 6080 | N | GLY | A | 1079 | 98.078 | 36.210 | 91.912 | 1.00 | 38.84 | 7 |
| ATOM | 6081 | CA | GLY | A | 1079 | 97.113 | 36.300 | 92.992 | 1.00 | 37.39 | 6 |
| ATOM | 6082 | C | GLY | A | 1079 | 96.844 | 37.741 | 93.394 | 1.00 | 39.24 | 6 |
| ATOM | 6083 | O | GLY | A | 1079 | 96.518 | 38.568 | 92.543 | 1.00 | 37.02 | 8 |
| ATOM | 6084 | N | TRP | A | 1080 | 96.995 | 38.041 | 94.685 | 1.00 | 39.16 | 7 |
| ATOM | 6085 | CA | TRP | A | 1080 | 96.764 | 39.380 | 95.226 | 1.00 | 40.17 | 6 |
| ATOM | 6086 | CB | TRP | A | 1080 | 96.129 | 39.284 | 96.617 | 1.00 | 40.13 | 6 |
| ATOM | 6087 | CG | TRP | A | 1080 | 94.760 | 38.715 | 96.631 | 1.00 | 42.16 | 6 |
| ATOM | 6088 | CD2 | TRP | A | 1080 | 93.550 | 39.392 | 96.271 | 1.00 | 41.45 | 6 |
| ATOM | 6089 | CE2 | TRP | A | 1080 | 92.492 | 38.468 | 96.424 | 1.00 | 43.14 | 6 |
| ATOM | 6090 | CE3 | TRP | A | 1080 | 93.258 | 40.693 | 95.834 | 1.00 | 39.85 | 6 |
| ATOM | 6091 | CD1 | TRP | A | 1080 | 94.401 | 37.443 | 96.978 | 1.00 | 43.22 | 6 |
| ATOM | 6092 | NE1 | TRP | A | 1080 | 93.039 | 37.287 | 96.857 | 1.00 | 44.87 | 7 |
| ATOM | 6093 | CZ2 | TRP | A | 1080 | 91.158 | 38.803 | 96.155 | 1.00 | 42.58 | 6 |
| ATOM | 6094 | CZ3 | TRP | A | 1080 | 91.931 | 41.026 | 95.567 | 1.00 | 41.26 | 6 |
| ATOM | 6095 | CH2 | TRP | A | 1080 | 90.898 | 40.081 | 95.729 | 1.00 | 41.20 | 6 |
| ATOM | 6096 | C | TRP | A | 1080 | 98.011 | 40.253 | 95.352 | 1.00 | 40.97 | 6 |
| ATOM | 6097 | O | TRP | A | 1080 | 97.914 | 41.415 | 95.764 | 1.00 | 41.95 | 8 |
| ATOM | 6098 | N | THR | A | 1081 | 99.171 | 39.702 | 95.010 | 1.00 | 39.61 | 7 |
| ATOM | 6099 | CA | THR | A | 1081 | 100.443 | 40.411 | 95.139 | 1.00 | 39.96 | 6 |
| ATOM | 6100 | CB | THR | A | 1081 | 101.584 | 39.598 | 94.511 | 1.00 | 41.50 | 6 |
| ATOM | 6101 | OG1 | THR | A | 1081 | 101.682 | 38.333 | 95.176 | 1.00 | 45.76 | 8 |
| ATOM | 6102 | CG2 | THR | A | 1081 | 102.913 | 40.338 | 94.649 | 1.00 | 44.46 | 6 |
| ATOM | 6103 | C | THR | A | 1081 | 100.514 | 41.832 | 94.583 | 1.00 | 40.96 | 6 |
| ATOM | 6104 | O | THR | A | 1081 | 100.896 | 42.765 | 95.296 | 1.00 | 41.20 | 8 |
| ATOM | 6105 | N | VAL | A | 1082 | 100.226 | 41.963 | 93.280 | 1.00 | 39.00 | 7 |
| ATOM | 6106 | CA | VAL | A | 1082 | 100.304 | 43.273 | 92.576 | 1.00 | 38.03 | 6 |
| ATOM | 6107 | CB | VAL | A | 1082 | 100.057 | 43.175 | 91.054 | 1.00 | 37.74 | 6 |
| ATOM | 6108 | CG1 | VAL | A | 1082 | 100.322 | 44.511 | 90.378 | 1.00 | 36.04 | 6 |
| ATOM | 6109 | CG2 | VAL | A | 1082 | 100.918 | 42.097 | 90.428 | 1.00 | 38.72 | 6 |
| ATOM | 6110 | C | VAL | A | 1082 | 99.275 | 44.207 | 93.066 | 1.00 | 38.08 | 6 |
| ATOM | 6111 | O | VAL | A | 1082 | 99.370 | 45.431 | 92.928 | 1.00 | 38.62 | 8 |
| ATOM | 6112 | N | GLN | A | 1083 | 98.286 | 43.636 | 93.641 | 1.00 | 38.04 | 7 |
| ATOM | 6113 | CA | GLN | A | 1083 | 97.244 | 44.439 | 94.154 | 1.00 | 37.09 | 6 |
| ATOM | 6114 | CB | GLN | A | 1083 | 95.927 | 43.682 | 94.113 | 1.00 | 36.47 | 6 |
| ATOM | 6115 | CG | GLN | A | 1083 | 94.999 | 44.199 | 93.063 | 1.00 | 35.34 | 6 |
| ATOM | 6116 | CD | GLN | A | 1083 | 93.900 | 45.001 | 93.687 | 1.00 | 35.22 | 6 |
| ATOM | 6117 | OE1 | GLN | A | 1083 | 93.037 | 44.457 | 94.378 | 1.00 | 34.15 | 8 |
| ATOM | 6118 | NE2 | GLN | A | 1083 | 93.734 | 46.302 | 93.584 | 1.00 | 29.69 | 7 |
| ATOM | 6119 | C | GLN | A | 1083 | 97.596 | 44.822 | 95.540 | 1.00 | 38.53 | 6 |
| ATOM | 6120 | O | GLN | A | 1083 | 97.444 | 45.985 | 95.904 | 1.00 | 37.03 | 8 |
| ATOM | 6121 | N | PHE | A | 1084 | 98.016 | 43.904 | 96.366 | 1.00 | 39.01 | 7 |
| ATOM | 6122 | CA | PHE | A | 1084 | 98.146 | 44.500 | 97.657 | 1.00 | 39.76 | 6 |
| ATOM | 6123 | CB | PHE | A | 1084 | 97.706 | 43.538 | 98.718 | 1.00 | 40.27 | 6 |
| ATOM | 6124 | CG | PHE | A | 1084 | 96.213 | 43.639 | 98.633 | 1.00 | 41.71 | 6 |
| ATOM | 6125 | CD1 | PHE | A | 1084 | 95.505 | 42.680 | 97.942 | 1.00 | 40.19 | 6 |
| ATOM | 6126 | CD2 | PHE | A | 1084 | 95.491 | 44.665 | 99.213 | 1.00 | 41.95 | 6 |
| ATOM | 6127 | CE1 | PHE | A | 1084 | 94.127 | 42.744 | 97.832 | 1.00 | 42.13 | 6 |
| ATOM | 6128 | CE2 | PHE | A | 1084 | 94.114 | 44.736 | 99.107 | 1.00 | 44.15 | 6 |
| ATOM | 6129 | CZ | PHE | A | 1084 | 93.415 | 43.774 | 98.416 | 1.00 | 41.24 | 6 |
| ATOM | 6130 | C | PHE | A | 1084 | 99.454 | 44.961 | 97.091 | 1.00 | 39.61 | 6 |
| ATOM | 6131 | O | PHE | A | 1084 | 99.726 | 44.700 | 95.913 | 1.00 | 39.02 | 8 |
| ATOM | 1 | N | MET | B | 201 | 53.741 | 61.791 | 99.131 | 1.00 | 51.28 | 7 |
| ATOM | 2 | CA | MET | B | 201 | 52.634 | 61.051 | 98.538 | 1.00 | 49.44 | 6 |
| ATOM | 3 | C | MET | B | 201 | 53.048 | 59.630 | 98.181 | 1.00 | 48.28 | 6 |
| ATOM | 4 | O | MET | B | 201 | 53.509 | 59.310 | 97.087 | 1.00 | 47.77 | 8 |
| ATOM | 5 | CB | MET | B | 201 | 52.101 | 61.744 | 97.303 | 1.00 | 50.50 | 6 |
| ATOM | 6 | CG | MET | B | 201 | 50.898 | 61.022 | 96.725 | 1.00 | 49.84 | 6 |
| ATOM | 7 | SD | MET | B | 201 | 50.316 | 61.763 | 95.202 | 1.00 | 49.51 | 16 |
| ATOM | 8 | CE | MET | B | 201 | 51.580 | 61.223 | 94.047 | 1.00 | 48.52 | 6 |
| ATOM | 9 | N | THR | B | 202 | 52.865 | 58.871 | 99.251 | 1.00 | 46.28 | 7 |
| ATOM | 10 | CA | THR | B | 202 | 53.086 | 57.459 | 99.345 | 1.00 | 45.23 | 6 |
| ATOM | 11 | C | THR | B | 202 | 52.490 | 56.806 | 98.123 | 1.00 | 43.28 | 6 |
| ATOM | 12 | O | THR | B | 202 | 51.377 | 57.141 | 97.702 | 1.00 | 40.67 | 8 |
| ATOM | 13 | CB | THR | B | 202 | 52.513 | 56.963 | 100.671 | 1.00 | 47.20 | 6 |
| ATOM | 14 | OG1 | THR | B | 202 | 53.005 | 57.777 | 101.737 | 1.00 | 48.61 | 8 |
| ATOM | 15 | CG2 | THR | B | 202 | 52.927 | 55.520 | 100.910 | 1.00 | 49.33 | 6 |
| ATOM | 16 | N | GLU | B | 203 | 53.254 | 55.872 | 97.573 | 1.00 | 41.53 | 7 |
| ATOM | 17 | CA | GLU | B | 203 | 52.875 | 55.192 | 96.330 | 1.00 | 38.67 | 6 |
| ATOM | 18 | C | GLU | B | 203 | 52.593 | 53.698 | 96.478 | 1.00 | 36.51 | 6 |
| ATOM | 19 | O | GLU | B | 203 | 53.359 | 52.967 | 97.117 | 1.00 | 35.10 | 8 |
| ATOM | 20 | CB | GLU | B | 203 | 53.985 | 55.452 | 95.313 | 1.00 | 38.84 | 6 |
| ATOM | 21 | CG | GLU | B | 203 | 53.722 | 54.907 | 93.932 | 1.00 | 42.79 | 6 |
| ATOM | 22 | CD | GLU | B | 203 | 54.829 | 55.235 | 92.936 | 1.00 | 44.78 | 6 |
| ATOM | 23 | OE1 | GLU | B | 203 | 54.897 | 56.379 | 92.469 | 1.00 | 47.37 | 8 |
| ATOM | 24 | OE2 | GLU | B | 203 | 55.617 | 54.314 | 92.635 | 1.00 | 46.62 | 8 |
| ATOM | 25 | N | TYR | B | 204 | 51.500 | 53.242 | 95.897 | 1.00 | 33.91 | 7 |
| ATOM | 26 | CA | TYR | B | 204 | 51.068 | 51.858 | 96.006 | 1.00 | 30.69 | 6 |
| ATOM | 27 | C | TYR | B | 204 | 51.101 | 51.162 | 94.640 | 1.00 | 28.79 | 6 |
| ATOM | 28 | O | TYR | B | 204 | 50.511 | 51.648 | 93.673 | 1.00 | 26.78 | 8 |
| ATOM | 29 | CB | TYR | B | 204 | 49.657 | 51.786 | 96.586 | 1.00 | 29.61 | 6 |
| ATOM | 30 | CG | TYR | B | 204 | 49.562 | 52.203 | 98.025 | 1.00 | 29.85 | 6 |
| ATOM | 31 | CD1 | TYR | B | 204 | 50.030 | 51.392 | 99.060 | 1.00 | 31.05 | 6 |
| ATOM | 32 | CD2 | TYR | B | 204 | 48.981 | 53.422 | 98.358 | 1.00 | 29.42 | 6 |
| ATOM | 33 | CE1 | TYR | B | 204 | 49.918 | 51.784 | 100.391 | 1.00 | 29.72 | 6 |
| ATOM | 34 | CE2 | TYR | B | 204 | 48.863 | 53.823 | 99.686 | 1.00 | 30.69 | 6 |
| ATOM | 35 | CZ | TYR | B | 204 | 49.332 | 52.998 | 100.696 | 1.00 | 31.20 | 6 |
| ATOM | 36 | OH | TYR | B | 204 | 49.225 | 53.408 | 102.011 | 1.00 | 35.67 | 8 |
| ATOM | 37 | N | LYS | B | 205 | 51.784 | 50.024 | 94.565 | 1.00 | 27.93 | 7 |
| ATOM | 38 | CA | LYS | B | 205 | 51.901 | 49.281 | 93.322 | 1.00 | 24.68 | 6 |
| ATOM | 39 | C | LYS | B | 205 | 50.954 | 48.089 | 93.314 | 1.00 | 21.57 | 6 |
| ATOM | 40 | O | LYS | B | 205 | 51.240 | 47.043 | 93.904 | 1.00 | 20.12 | 8 |
| ATOM | 41 | CB | LYS | B | 205 | 53.347 | 48.840 | 93.105 | 1.00 | 28.19 | 6 |
| ATOM | 42 | CG | LYS | B | 205 | 54.331 | 49.995 | 93.001 | 1.00 | 34.48 | 6 |
| ATOM | 43 | CD | LYS | B | 205 | 55.704 | 49.514 | 92.564 | 1.00 | 40.70 | 6 |
| ATOM | 44 | CE | LYS | B | 205 | 56.688 | 50.669 | 92.467 | 1.00 | 43.84 | 6 |
| ATOM | 45 | NZ | LYS | B | 205 | 58.037 | 50.215 | 92.034 | 1.00 | 49.59 | 7 |
| ATOM | 46 | N | LEU | B | 206 | 49.808 | 48.278 | 92.659 | 1.00 | 18.65 | 7 |
| ATOM | 47 | CA | LEU | B | 206 | 48.776 | 47.255 | 92.538 | 1.00 | 15.19 | 6 |
| ATOM | 48 | C | LEU | B | 206 | 48.889 | 46.505 | 91.215 | 1.00 | 14.14 | 6 |
| ATOM | 49 | O | LEU | B | 206 | 49.223 | 47.089 | 90.181 | 1.00 | 10.75 | 8 |
| ATOM | 50 | CB | LEU | B | 206 | 47.380 | 47.883 | 92.635 | 1.00 | 13.89 | 6 |
| ATOM | 51 | CG | LEU | B | 206 | 46.923 | 48.491 | 93.964 | 1.00 | 18.13 | 6 |
| ATOM | 52 | CD1 | LEU | B | 206 | 47.699 | 49.747 | 94.263 | 1.00 | 14.35 | 6 |
| ATOM | 53 | CD2 | LEU | B | 206 | 45.437 | 48.810 | 93.905 | 1.00 | 17.76 | 6 |
| ATOM | 54 | N | VAL | B | 207 | 48.609 | 45.206 | 91.259 | 1.00 | 11.52 | 7 |
| ATOM | 55 | CA | VAL | B | 207 | 48.658 | 44.370 | 90.068 | 1.00 | 11.37 | 6 |
| ATOM | 56 | C | VAL | B | 207 | 47.338 | 43.609 | 89.930 | 1.00 | 11.18 | 6 |
| ATOM | 57 | O | VAL | B | 207 | 46.879 | 42.960 | 90.875 | 1.00 | 9.73 | 8 |
| ATOM | 58 | CB | VAL | B | 207 | 49.841 | 43.363 | 90.124 | 1.00 | 11.42 | 6 |
| ATOM | 59 | CG1 | VAL | B | 207 | 49.956 | 42.606 | 88.794 | 1.00 | 8.29 | 6 |
| ATOM | 60 | CG2 | VAL | B | 207 | 51.147 | 44.095 | 90.418 | 1.00 | 8.29 | 6 |
| ATOM | 61 | N | VAL | B | 208 | 46.718 | 43.732 | 88.759 | 1.00 | 10.37 | 7 |
| ATOM | 62 | CA | VAL | B | 208 | 45.448 | 43.063 | 88.478 | 1.00 | 9.27 | 6 |
| ATOM | 63 | C | VAL | B | 208 | 45.743 | 41.787 | 87.692 | 1.00 | 11.11 | 6 |
| ATOM | 64 | O | VAL | B | 208 | 46.208 | 41.842 | 86.546 | 1.00 | 11.80 | 8 |
| ATOM | 65 | CB | VAL | B | 208 | 44.495 | 43.971 | 87.664 | 1.00 | 6.10 | 6 |
| ATOM | 66 | CG1 | VAL | B | 208 | 43.099 | 43.378 | 87.625 | 1.00 | 4.20 | 6 |
| ATOM | 67 | CG2 | VAL | B | 208 | 44.460 | 45.370 | 88.268 | 1.00 | 8.22 | 6 |
| ATOM | 68 | N | VAL | B | 209 | 45.489 | 40.643 | 88.324 | 1.00 | 9.18 | 7 |
| ATOM | 69 | CA | VAL | B | 209 | 45.743 | 39.341 | 87.713 | 1.00 | 9.00 | 6 |
| ATOM | 70 | C | VAL | B | 209 | 44.486 | 38.479 | 87.566 | 1.00 | 8.88 | 6 |
| ATOM | 71 | O | VAL | B | 209 | 43.485 | 38.696 | 88.246 | 1.00 | 6.64 | 8 |
| ATOM | 72 | CB | VAL | B | 209 | 46.802 | 38.545 | 88.518 | 1.00 | 6.04 | 6 |
| ATOM | 73 | CG1 | VAL | B | 209 | 48.118 | 39.303 | 88.555 | 1.00 | 4.79 | 6 |
| ATOM | 74 | CG2 | VAL | B | 209 | 46.308 | 38.283 | 89.925 | 1.00 | 2.58 | 6 |
| ATOM | 75 | N | GLY | B | 210 | 44.557 | 37.499 | 86.667 | 1.00 | 8.62 | 7 |
| ATOM | 76 | CA | GLY | B | 210 | 43.434 | 36.607 | 86.432 | 1.00 | 7.31 | 6 |
| ATOM | 77 | C | GLY | B | 210 | 43.402 | 36.110 | 85.000 | 1.00 | 6.94 | 6 |
| ATOM | 78 | O | GLY | B | 210 | 44.090 | 36.653 | 84.134 | 1.00 | 4.02 | 8 |
| ATOM | 79 | N | ALA | B | 211 | 42.596 | 35.082 | 84.748 | 1.00 | 7.14 | 7 |
| ATOM | 80 | CA | ALA | B | 211 | 42.469 | 34.492 | 83.417 | 1.00 | 7.36 | 6 |
| ATOM | 81 | C | ALA | B | 211 | 41.976 | 35.496 | 82.377 | 1.00 | 7.24 | 6 |
| ATOM | 82 | O | ALA | B | 211 | 41.410 | 36.537 | 82.715 | 1.00 | 8.46 | 8 |
| ATOM | 83 | CB | ALA | B | 211 | 41.538 | 33.276 | 83.465 | 1.00 | 6.62 | 6 |
| ATOM | 84 | N | VAL | B | 212 | 42.166 | 35.163 | 81.110 | 1.00 | 8.63 | 7 |
| ATOM | 85 | CA | VAL | B | 212 | 41.794 | 36.049 | 80.012 | 1.00 | 7.16 | 6 |
| ATOM | 86 | C | VAL | B | 212 | 40.275 | 36.231 | 79.957 | 1.00 | 7.92 | 6 |
| ATOM | 87 | O | VAL | B | 212 | 39.495 | 35.315 | 80.156 | 1.00 | 6.09 | 8 |
| ATOM | 88 | CB | VAL | B | 212 | 42.326 | 35.521 | 78.665 | 1.00 | 20.00 | 6 |
| ATOM | 89 | CG1 | VAL | B | 212 | 41.635 | 34.217 | 78.293 | 1.00 | 20.00 | 6 |
| ATOM | 90 | CG2 | VAL | B | 212 | 42.143 | 36.561 | 77.571 | 1.00 | 20.00 | 6 |
| ATOM | 91 | N | GLY | B | 213 | 39.886 | 37.485 | 79.674 | 1.00 | 9.19 | 7 |
| ATOM | 92 | CA | GLY | B | 213 | 38.482 | 37.829 | 79.530 | 1.00 | 8.57 | 6 |
| ATOM | 93 | C | GLY | B | 213 | 37.641 | 38.012 | 80.777 | 1.00 | 8.47 | 6 |
| ATOM | 94 | O | GLY | B | 213 | 36.440 | 38.265 | 80.665 | 1.00 | 9.56 | 8 |
| ATOM | 95 | N | VAL | B | 214 | 38.243 | 37.906 | 81.959 | 1.00 | 7.99 | 7 |
| ATOM | 96 | CA | VAL | B | 214 | 37.446 | 38.064 | 83.199 | 1.00 | 6.82 | 6 |
| ATOM | 97 | C | VAL | B | 214 | 37.057 | 39.503 | 83.510 | 1.00 | 8.46 | 6 |
| ATOM | 98 | O | VAL | B | 214 | 36.145 | 39.725 | 84.301 | 1.00 | 6.80 | 8 |
| ATOM | 99 | CB | VAL | B | 214 | 38.208 | 37.438 | 84.413 | 1.00 | 7.50 | 6 |
| ATOM | 100 | CG1 | VAL | B | 214 | 38.415 | 35.957 | 84.202 | 1.00 | 5.57 | 6 |
| ATOM | 101 | CG2 | VAL | B | 214 | 39.542 | 38.140 | 84.615 | 1.00 | 3.32 | 6 |
| ATOM | 102 | N | GLY | B | 215 | 37.731 | 40.472 | 82.924 | 1.00 | 7.60 | 7 |
| ATOM | 103 | CA | GLY | B | 215 | 37.397 | 41.867 | 83.155 | 1.00 | 6.99 | 6 |
| ATOM | 104 | C | GLY | B | 215 | 38.514 | 42.649 | 83.857 | 1.00 | 8.79 | 6 |
| ATOM | 105 | O | GLY | B | 215 | 38.246 | 43.636 | 84.539 | 1.00 | 10.93 | 8 |
| ATOM | 106 | N | LYS | B | 216 | 39.793 | 42.224 | 83.683 | 1.00 | 9.81 | 7 |
| ATOM | 107 | CA | LYS | B | 216 | 40.923 | 42.926 | 84.323 | 1.00 | 10.11 | 6 |
| ATOM | 108 | C | LYS | B | 216 | 40.985 | 44.404 | 83.943 | 1.00 | 9.57 | 6 |
| ATOM | 109 | O | LYS | B | 216 | 40.910 | 45.308 | 84.780 | 1.00 | 7.71 | 8 |
| ATOM | 110 | CB | LYS | B | 216 | 42.248 | 42.278 | 83.939 | 1.00 | 8.73 | 6 |
| ATOM | 111 | CG | LYS | B | 216 | 42.284 | 40.775 | 84.096 | 1.00 | 12.18 | 6 |
| ATOM | 112 | CD | LYS | B | 216 | 43.267 | 40.133 | 83.119 | 1.00 | 12.29 | 6 |
| ATOM | 113 | CE | LYS | B | 216 | 43.812 | 38.812 | 83.665 | 1.00 | 11.54 | 6 |
| ATOM | 114 | NZ | LYS | B | 216 | 43.286 | 37.637 | 82.914 | 1.00 | 11.90 | 7 |
| ATOM | 115 | N | SER | B | 217 | 41.110 | 44.600 | 82.629 | 1.00 | 8.65 | 7 |
| ATOM | 116 | CA | SER | B | 217 | 41.196 | 45.880 | 81.944 | 1.00 | 8.85 | 6 |
| ATOM | 117 | C | SER | B | 217 | 39.975 | 46.747 | 82.198 | 1.00 | 7.85 | 6 |
| ATOM | 118 | O | SER | B | 217 | 40.098 | 47.924 | 82.523 | 1.00 | 9.51 | 8 |
| ATOM | 119 | CB | SER | B | 217 | 41.370 | 45.677 | 80.435 | 1.00 | 9.24 | 6 |
| ATOM | 120 | OG | SER | B | 217 | 42.533 | 44.926 | 80.161 | 1.00 | 9.51 | 8 |
| ATOM | 121 | N | ALA | B | 218 | 38.797 | 46.151 | 82.058 | 1.00 | 7.69 | 7 |
| ATOM | 122 | CA | ALA | B | 218 | 37.542 | 46.861 | 82.279 | 1.00 | 9.30 | 6 |
| ATOM | 123 | C | ALA | B | 218 | 37.498 | 47.439 | 83.692 | 1.00 | 9.53 | 6 |
| ATOM | 124 | O | ALA | B | 218 | 37.121 | 48.598 | 83.884 | 1.00 | 11.33 | 8 |
| ATOM | 125 | CB | ALA | B | 218 | 36.370 | 45.928 | 82.056 | 1.00 | 4.97 | 6 |
| ATOM | 126 | N | LEU | B | 219 | 37.907 | 46.625 | 84.665 | 1.00 | 8.11 | 7 |
| ATOM | 127 | CA | LEU | B | 219 | 37.948 | 46.996 | 86.081 | 1.00 | 8.83 | 6 |
| ATOM | 128 | C | LEU | B | 219 | 38.904 | 48.163 | 86.307 | 1.00 | 9.77 | 6 |
| ATOM | 129 | O | LEU | B | 219 | 38.546 | 49.164 | 86.931 | 1.00 | 9.84 | 8 |
| ATOM | 130 | CB | LEU | B | 219 | 38.401 | 45.788 | 86.904 | 1.00 | 7.47 | 6 |
| ATOM | 131 | CG | LEU | B | 219 | 37.569 | 45.312 | 88.095 | 1.00 | 13.16 | 6 |
| ATOM | 132 | CD1 | LEU | B | 219 | 36.088 | 45.532 | 87.861 | 1.00 | 8.63 | 6 |
| ATOM | 133 | CD2 | LEU | B | 219 | 37.864 | 43.838 | 88.354 | 1.00 | 10.01 | 6 |
| ATOM | 134 | N | THR | B | 220 | 40.112 | 48.035 | 85.765 | 1.00 | 9.83 | 7 |
| ATOM | 135 | CA | THR | B | 220 | 41.146 | 49.058 | 85.893 | 1.00 | 6.71 | 6 |
| ATOM | 136 | C | THR | B | 220 | 40.708 | 50.380 | 85.263 | 1.00 | 9.11 | 6 |
| ATOM | 137 | O | THR | B | 220 | 40.793 | 51.437 | 85.891 | 1.00 | 7.07 | 8 |
| ATOM | 138 | CB | THR | B | 220 | 42.454 | 48.596 | 85.234 | 1.00 | 9.38 | 6 |
| ATOM | 139 | OG1 | THR | B | 220 | 42.893 | 47.377 | 85.847 | 1.00 | 7.13 | 8 |
| ATOM | 140 | CG2 | THR | B | 220 | 43.537 | 49.662 | 85.384 | 1.00 | 7.15 | 6 |
| ATOM | 141 | N | ILE | B | 221 | 40.231 | 50.309 | 84.023 | 1.00 | 10.23 | 7 |
| ATOM | 142 | CA | ILE | B | 221 | 39.772 | 51.486 | 83.294 | 1.00 | 11.37 | 6 |
| ATOM | 143 | C | ILE | B | 221 | 38.581 | 52.150 | 83.982 | 1.00 | 13.77 | 6 |
| ATOM | 144 | O | ILE | B | 221 | 38.449 | 53.372 | 83.950 | 1.00 | 17.70 | 8 |
| ATOM | 145 | CB | ILE | B | 221 | 39.432 | 51.116 | 81.835 | 1.00 | 11.83 | 6 |
| ATOM | 146 | CG1 | ILE | B | 221 | 40.721 | 50.742 | 81.092 | 1.00 | 9.78 | 6 |
| ATOM | 147 | CG2 | ILE | B | 221 | 38.712 | 52.264 | 81.138 | 1.00 | 13.38 | 6 |
| ATOM | 148 | CD1 | ILE | B | 221 | 40.506 | 49.908 | 79.851 | 1.00 | 13.87 | 6 |
| ATOM | 149 | N | GLN | B | 222 | 37.723 | 51.351 | 84.609 | 1.00 | 13.58 | 7 |
| ATOM | 150 | CA | GLN | B | 222 | 36.567 | 51.892 | 85.312 | 1.00 | 14.08 | 6 |
| ATOM | 151 | C | GLN | B | 222 | 37.041 | 52.745 | 86.485 | 1.00 | 16.48 | 6 |
| ATOM | 152 | O | GLN | B | 222 | 36.529 | 53.841 | 86.712 | 1.00 | 16.85 | 8 |
| ATOM | 153 | CB | GLN | B | 222 | 35.660 | 50.768 | 85.815 | 1.00 | 14.15 | 6 |
| ATOM | 154 | CG | GLN | B | 222 | 34.392 | 51.245 | 86.520 | 1.00 | 15.60 | 6 |
| ATOM | 155 | CD | GLN | B | 222 | 33.469 | 52.070 | 85.626 | 1.00 | 16.95 | 6 |
| ATOM | 156 | OE1 | GLN | B | 222 | 33.520 | 53.299 | 85.634 | 1.00 | 16.90 | 8 |
| ATOM | 157 | NE2 | GLN | B | 222 | 32.602 | 51.395 | 84.876 | 1.00 | 14.47 | 7 |
| ATOM | 158 | N | LEU | B | 223 | 38.057 | 52.260 | 87.193 | 1.00 | 17.36 | 7 |
| ATOM | 159 | CA | LEU | B | 223 | 38.609 | 52.975 | 88.342 | 1.00 | 18.18 | 6 |
| ATOM | 160 | C | LEU | B | 223 | 39.400 | 54.222 | 87.914 | 1.00 | 17.63 | 6 |
| ATOM | 161 | O | LEU | B | 223 | 39.193 | 55.312 | 88.440 | 1.00 | 19.17 | 8 |
| ATOM | 162 | CB | LEU | B | 223 | 39.513 | 52.050 | 89.162 | 1.00 | 17.42 | 6 |
| ATOM | 163 | CG | LEU | B | 223 | 40.070 | 52.699 | 90.430 | 1.00 | 20.15 | 6 |
| ATOM | 164 | CD1 | LEU | B | 223 | 39.113 | 52.514 | 91.599 | 1.00 | 18.81 | 6 |
| ATOM | 165 | CD2 | LEU | B | 223 | 41.449 | 52.150 | 90.769 | 1.00 | 18.18 | 6 |
| ATOM | 166 | N | ILE | B | 224 | 40.312 | 54.047 | 86.929 | 1.00 | 17.04 | 7 |
| ATOM | 167 | CA | ILE | B | 224 | 41.167 | 55.164 | 86.495 | 1.00 | 16.09 | 6 |
| ATOM | 168 | C | ILE | B | 224 | 40.386 | 56.265 | 85.727 | 1.00 | 19.03 | 6 |
| ATOM | 169 | O | ILE | B | 224 | 40.615 | 57.431 | 85.994 | 1.00 | 18.33 | 8 |
| ATOM | 170 | CB | ILE | B | 224 | 42.384 | 54.616 | 85.724 | 1.00 | 16.45 | 6 |
| ATOM | 171 | CG1 | ILE | B | 224 | 43.125 | 53.589 | 86.589 | 1.00 | 15.55 | 6 |
| ATOM | 172 | CG2 | ILE | B | 224 | 43.323 | 55.752 | 85.324 | 1.00 | 13.57 | 6 |
| ATOM | 173 | CD1 | ILE | B | 224 | 43.833 | 54.198 | 87.782 | 1.00 | 17.84 | 6 |
| ATOM | 174 | N | GLN | B | 225 | 39.488 | 55.925 | 84.770 | 1.00 | 18.73 | 7 |
| ATOM | 175 | CA | GLN | B | 225 | 38.731 | 56.946 | 83.983 | 1.00 | 20.33 | 6 |
| ATOM | 176 | C | GLN | B | 225 | 37.269 | 57.034 | 84.397 | 1.00 | 21.59 | 6 |
| ATOM | 177 | O | GLN | B | 225 | 36.701 | 58.126 | 84.358 | 1.00 | 19.80 | 8 |
| ATOM | 178 | CB | GLN | B | 225 | 38.784 | 56.642 | 82.446 | 1.00 | 20.16 | 6 |
| ATOM | 179 | CG | GLN | B | 225 | 39.711 | 57.525 | 81.585 | 1.00 | 20.02 | 6 |
| ATOM | 180 | CD | GLN | B | 225 | 39.427 | 57.507 | 80.079 | 1.00 | 22.47 | 6 |
| ATOM | 181 | OE1 | GLN | B | 225 | 38.362 | 57.048 | 79.638 | 1.00 | 24.08 | 8 |
| ATOM | 182 | NE2 | GLN | B | 225 | 40.235 | 57.943 | 79.134 | 1.00 | 22.96 | 7 |
| ATOM | 183 | N | ASN | B | 226 | 36.660 | 55.953 | 84.791 | 1.00 | 24.10 | 7 |
| ATOM | 184 | CA | ASN | B | 226 | 35.275 | 56.144 | 85.062 | 1.00 | 28.68 | 6 |
| ATOM | 185 | C | ASN | B | 226 | 34.582 | 55.986 | 83.737 | 1.00 | 31.72 | 6 |
| ATOM | 186 | O | ASN | B | 226 | 34.201 | 56.952 | 83.074 | 1.00 | 33.20 | 8 |
| ATOM | 187 | CB | ASN | B | 226 | 35.009 | 57.539 | 85.626 | 1.00 | 33.17 | 6 |
| ATOM | 188 | CG | ASN | B | 226 | 33.643 | 57.609 | 86.225 | 1.00 | 39.89 | 6 |
| ATOM | 189 | OD1 | ASN | B | 226 | 33.396 | 58.439 | 87.108 | 1.00 | 42.01 | 8 |
| ATOM | 190 | ND2 | ASN | B | 226 | 32.731 | 56.741 | 85.771 | 1.00 | 41.16 | 7 |
| ATOM | 191 | N | HIS | B | 227 | 34.424 | 54.757 | 83.380 | 1.00 | 30.85 | 7 |
| ATOM | 192 | CA | HIS | B | 227 | 33.788 | 54.490 | 82.161 | 1.00 | 29.41 | 6 |
| ATOM | 193 | C | HIS | B | 227 | 34.030 | 53.072 | 81.755 | 1.00 | 26.28 | 6 |
| ATOM | 194 | O | HIS | B | 227 | 35.033 | 52.448 | 82.110 | 1.00 | 21.97 | 8 |
| ATOM | 195 | CB | HIS | B | 227 | 34.215 | 55.533 | 81.131 | 1.00 | 32.75 | 6 |
| ATOM | 196 | CG | HIS | B | 227 | 34.911 | 54.992 | 79.861 | 1.00 | 36.45 | 6 |
| ATOM | 197 | ND1 | HIS | B | 227 | 34.213 | 54.660 | 78.718 | 1.00 | 39.88 | 7 |
| ATOM | 198 | CD2 | HIS | B | 227 | 36.218 | 54.790 | 79.574 | 1.00 | 38.48 | 6 |
| ATOM | 199 | CE1 | HIS | B | 227 | 35.064 | 54.275 | 77.783 | 1.00 | 41.01 | 6 |
| ATOM | 200 | NE2 | HIS | B | 227 | 36.286 | 54.345 | 78.276 | 1.00 | 39.45 | 7 |
| ATOM | 201 | N | PHE | B | 228 | 33.014 | 52.585 | 81.002 | 1.00 | 23.47 | 7 |
| ATOM | 202 | CA | PHE | B | 228 | 33.007 | 51.207 | 80.562 | 1.00 | 22.82 | 6 |
| ATOM | 203 | C | PHE | B | 228 | 32.696 | 50.960 | 79.113 | 1.00 | 24.50 | 6 |
| ATOM | 204 | O | PHE | B | 228 | 31.753 | 51.424 | 78.477 | 1.00 | 26.79 | 8 |
| ATOM | 205 | CB | PHE | B | 228 | 32.097 | 50.400 | 81.445 | 1.00 | 19.66 | 6 |
| ATOM | 206 | CG | PHE | B | 228 | 31.927 | 48.995 | 80.923 | 1.00 | 14.82 | 6 |
| ATOM | 207 | CD1 | PHE | B | 228 | 30.879 | 48.716 | 80.082 | 1.00 | 15.86 | 6 |
| ATOM | 208 | CD2 | PHE | B | 228 | 32.815 | 47.987 | 81.271 | 1.00 | 14.34 | 6 |
| ATOM | 209 | CE1 | PHE | B | 228 | 30.663 | 47.452 | 79.593 | 1.00 | 9.36 | 6 |
| ATOM | 210 | CE2 | PHE | B | 228 | 32.608 | 46.693 | 80.789 | 1.00 | 11.60 | 6 |
| ATOM | 211 | CZ | PHE | B | 228 | 31.526 | 46.428 | 79.946 | 1.00 | 10.60 | 6 |
| ATOM | 212 | N | VAL | B | 229 | 33.616 | 50.157 | 78.732 | 1.00 | 25.32 | 7 |
| ATOM | 213 | CA | VAL | B | 229 | 33.873 | 49.609 | 77.453 | 1.00 | 27.53 | 6 |
| ATOM | 214 | C | VAL | B | 229 | 33.023 | 48.483 | 77.082 | 1.00 | 28.42 | 6 |
| ATOM | 215 | O | VAL | B | 229 | 32.510 | 47.733 | 77.906 | 1.00 | 28.15 | 8 |
| ATOM | 216 | CB | VAL | B | 229 | 35.272 | 48.949 | 77.470 | 1.00 | 28.47 | 6 |
| ATOM | 217 | CG1 | VAL | B | 229 | 36.332 | 49.936 | 77.936 | 1.00 | 27.03 | 6 |
| ATOM | 218 | CG2 | VAL | B | 229 | 35.251 | 47.728 | 78.383 | 1.00 | 31.22 | 6 |
| ATOM | 219 | N | ASP | B | 230 | 32.890 | 48.367 | 75.835 | 1.00 | 27.38 | 7 |
| ATOM | 220 | CA | ASP | B | 230 | 32.314 | 47.180 | 75.402 | 1.00 | 27.07 | 6 |
| ATOM | 221 | C | ASP | B | 230 | 33.231 | 46.783 | 74.361 | 1.00 | 25.75 | 6 |
| ATOM | 222 | O | ASP | B | 230 | 33.062 | 47.165 | 73.205 | 1.00 | 25.41 | 8 |
| ATOM | 223 | CB | ASP | B | 230 | 30.926 | 47.257 | 74.843 | 1.00 | 29.51 | 6 |
| ATOM | 224 | CG | ASP | B | 230 | 30.433 | 45.812 | 74.766 | 1.00 | 32.34 | 6 |
| ATOM | 225 | OD1 | ASP | B | 230 | 30.808 | 45.094 | 73.819 | 1.00 | 37.01 | 8 |
| ATOM | 226 | OD2 | ASP | B | 230 | 29.674 | 45.409 | 75.657 | 1.00 | 37.81 | 8 |
| ATOM | 227 | N | GLU | B | 231 | 34.211 | 46.049 | 74.704 | 1.00 | 24.23 | 7 |
| ATOM | 228 | CA | GLU | B | 231 | 35.011 | 45.751 | 73.584 | 1.00 | 21.88 | 6 |
| ATOM | 229 | C | GLU | B | 231 | 36.034 | 44.814 | 73.923 | 1.00 | 20.00 | 6 |
| ATOM | 230 | O | GLU | B | 231 | 36.269 | 44.426 | 75.061 | 1.00 | 19.03 | 8 |
| ATOM | 231 | CB | GLU | B | 231 | 35.685 | 46.971 | 73.009 | 1.00 | 23.67 | 6 |
| ATOM | 232 | CG | GLU | B | 231 | 34.894 | 48.247 | 73.159 | 1.00 | 28.14 | 6 |
| ATOM | 233 | CD | GLU | B | 231 | 35.658 | 49.442 | 72.604 | 1.00 | 33.02 | 6 |
| ATOM | 234 | OE1 | GLU | B | 231 | 36.906 | 49.378 | 72.547 | 1.00 | 20.00 | 8 |
| ATOM | 235 | OE2 | GLU | B | 231 | 35.009 | 50.427 | 72.218 | 1.00 | 20.00 | 8 |
| ATOM | 236 | N | TYR | B | 232 | 36.666 | 44.368 | 72.924 | 1.00 | 17.58 | 7 |
| ATOM | 237 | CA | TYR | B | 232 | 37.616 | 43.481 | 73.357 | 1.00 | 15.09 | 6 |
| ATOM | 238 | C | TYR | B | 232 | 38.871 | 43.648 | 72.639 | 1.00 | 14.72 | 6 |
| ATOM | 239 | O | TYR | B | 232 | 39.037 | 43.446 | 71.431 | 1.00 | 13.19 | 8 |
| ATOM | 240 | CB | TYR | B | 232 | 37.020 | 42.137 | 73.413 | 1.00 | 15.62 | 6 |
| ATOM | 241 | CG | TYR | B | 232 | 37.999 | 41.319 | 74.146 | 1.00 | 15.84 | 6 |
| ATOM | 242 | CD1 | TYR | B | 232 | 38.312 | 40.117 | 73.588 | 1.00 | 16.94 | 6 |
| ATOM | 243 | CD2 | TYR | B | 232 | 38.626 | 41.729 | 75.316 | 1.00 | 18.16 | 6 |
| ATOM | 244 | CE1 | TYR | B | 232 | 39.188 | 39.283 | 74.186 | 1.00 | 14.73 | 6 |
| ATOM | 245 | CE2 | TYR | B | 232 | 39.531 | 40.903 | 75.925 | 1.00 | 16.00 | 6 |
| ATOM | 246 | CZ | TYR | B | 232 | 39.788 | 39.685 | 75.357 | 1.00 | 15.50 | 6 |
| ATOM | 247 | OH | TYR | B | 232 | 40.689 | 38.837 | 75.968 | 1.00 | 15.37 | 8 |
| ATOM | 248 | N | ASP | B | 233 | 39.762 | 44.057 | 73.501 | 1.00 | 14.46 | 7 |
| ATOM | 249 | CA | ASP | B | 233 | 41.066 | 44.495 | 73.172 | 1.00 | 13.56 | 6 |
| ATOM | 250 | C | ASP | B | 233 | 42.138 | 43.998 | 74.132 | 1.00 | 11.24 | 6 |
| ATOM | 251 | O | ASP | B | 233 | 42.402 | 44.539 | 75.213 | 1.00 | 10.70 | 8 |
| ATOM | 252 | CB | ASP | B | 233 | 40.938 | 46.009 | 73.048 | 1.00 | 13.01 | 6 |
| ATOM | 253 | CG | ASP | B | 233 | 42.210 | 46.800 | 72.901 | 1.00 | 15.77 | 6 |
| ATOM | 254 | OD1 | ASP | B | 233 | 43.211 | 46.233 | 72.406 | 1.00 | 19.40 | 8 |
| ATOM | 255 | OD2 | ASP | B | 233 | 42.214 | 47.985 | 73.288 | 1.00 | 17.35 | 8 |
| ATOM | 256 | N | PRO | B | 234 | 42.709 | 42.911 | 73.629 | 1.00 | 10.67 | 7 |
| ATOM | 257 | CA | PRO | B | 234 | 43.742 | 42.168 | 74.324 | 1.00 | 8.90 | 6 |
| ATOM | 258 | C | PRO | B | 234 | 44.865 | 42.970 | 74.873 | 1.00 | 9.38 | 6 |
| ATOM | 259 | O | PRO | B | 234 | 45.531 | 43.817 | 74.274 | 1.00 | 7.82 | 8 |
| ATOM | 260 | CB | PRO | B | 234 | 44.032 | 41.004 | 73.389 | 1.00 | 11.52 | 6 |
| ATOM | 261 | CG | PRO | B | 234 | 42.672 | 40.705 | 72.876 | 1.00 | 8.85 | 6 |
| ATOM | 262 | CD | PRO | B | 234 | 41.833 | 41.948 | 72.936 | 1.00 | 8.82 | 6 |
| ATOM | 263 | N | THR | B | 235 | 45.004 | 42.584 | 76.094 | 1.00 | 9.48 | 7 |
| ATOM | 264 | CA | THR | B | 235 | 45.897 | 43.076 | 77.061 | 1.00 | 8.60 | 6 |
| ATOM | 265 | C | THR | B | 235 | 47.251 | 42.320 | 77.106 | 1.00 | 8.10 | 6 |
| ATOM | 266 | O | THR | B | 235 | 47.284 | 41.166 | 77.517 | 1.00 | 9.36 | 8 |
| ATOM | 267 | CB | THR | B | 235 | 45.176 | 42.960 | 78.394 | 1.00 | 6.31 | 6 |
| ATOM | 268 | OG1 | THR | B | 235 | 43.767 | 43.206 | 78.195 | 1.00 | 7.96 | 8 |
| ATOM | 269 | CG2 | THR | B | 235 | 45.743 | 43.964 | 79.390 | 1.00 | 2.14 | 6 |
| ATOM | 270 | N | ILE | B | 236 | 48.346 | 42.945 | 76.689 | 1.00 | 7.23 | 7 |
| ATOM | 271 | CA | ILE | B | 236 | 49.693 | 42.326 | 76.803 | 1.00 | 7.94 | 6 |
| ATOM | 272 | C | ILE | B | 236 | 50.250 | 42.697 | 78.184 | 1.00 | 9.45 | 6 |
| ATOM | 273 | O | ILE | B | 236 | 50.585 | 41.852 | 78.999 | 1.00 | 9.93 | 8 |
| ATOM | 274 | CB | ILE | B | 236 | 50.684 | 42.852 | 75.730 | 1.00 | 6.39 | 6 |
| ATOM | 275 | CG1 | ILE | B | 236 | 50.356 | 42.274 | 74.349 | 1.00 | 6.36 | 6 |
| ATOM | 276 | CG2 | ILE | B | 236 | 52.129 | 42.515 | 76.106 | 1.00 | 6.69 | 6 |
| ATOM | 277 | CD1 | ILE | B | 236 | 51.337 | 42.672 | 73.276 | 1.00 | 10.80 | 6 |
| ATOM | 278 | N | GLU | B | 237 | 50.305 | 44.018 | 78.405 | 1.00 | 9.32 | 7 |
| ATOM | 279 | CA | GLU | B | 237 | 50.707 | 44.640 | 79.644 | 1.00 | 10.29 | 6 |
| ATOM | 280 | C | GLU | B | 237 | 50.503 | 46.147 | 79.579 | 1.00 | 10.56 | 6 |
| ATOM | 281 | O | GLU | B | 237 | 51.213 | 46.838 | 78.857 | 1.00 | 11.70 | 8 |
| ATOM | 282 | CB | GLU | B | 237 | 52.162 | 44.329 | 80.023 | 1.00 | 11.79 | 6 |
| ATOM | 283 | CG | GLU | B | 237 | 52.516 | 44.886 | 81.406 | 1.00 | 17.72 | 6 |
| ATOM | 284 | CD | GLU | B | 237 | 54.008 | 44.980 | 81.676 | 1.00 | 17.71 | 6 |
| ATOM | 285 | OE1 | GLU | B | 237 | 54.689 | 45.839 | 81.050 | 1.00 | 22.77 | 8 |
| ATOM | 286 | OE2 | GLU | B | 237 | 54.497 | 44.194 | 82.509 | 1.00 | 18.09 | 8 |
| ATOM | 287 | N | ASP | B | 238 | 49.536 | 46.621 | 80.340 | 1.00 | 12.33 | 7 |
| ATOM | 288 | CA | ASP | B | 238 | 49.255 | 48.057 | 80.374 | 1.00 | 11.37 | 6 |
| ATOM | 289 | C | ASP | B | 238 | 49.496 | 48.571 | 81.773 | 1.00 | 12.68 | 6 |
| ATOM | 290 | O | ASP | B | 238 | 49.258 | 47.866 | 82.763 | 1.00 | 13.32 | 8 |
| ATOM | 291 | CB | ASP | B | 238 | 47.808 | 48.365 | 79.962 | 1.00 | 12.06 | 6 |
| ATOM | 292 | CG | ASP | B | 238 | 47.483 | 47.852 | 78.575 | 1.00 | 14.27 | 6 |
| ATOM | 293 | OD1 | ASP | B | 238 | 48.417 | 47.599 | 77.781 | 1.00 | 10.32 | 8 |
| ATOM | 294 | OD2 | ASP | B | 238 | 46.280 | 47.711 | 78.276 | 1.00 | 14.55 | 8 |
| ATOM | 295 | N | SER | B | 239 | 49.956 | 49.789 | 81.873 | 1.00 | 12.94 | 7 |
| ATOM | 296 | CA | SER | B | 239 | 50.161 | 50.386 | 83.192 | 1.00 | 13.42 | 6 |
| ATOM | 297 | C | SER | B | 239 | 49.141 | 51.528 | 83.408 | 1.00 | 12.55 | 6 |
| ATOM | 298 | O | SER | B | 239 | 48.487 | 51.959 | 82.458 | 1.00 | 12.18 | 8 |
| ATOM | 299 | CB | SER | B | 239 | 51.595 | 50.882 | 83.343 | 1.00 | 15.17 | 6 |
| ATOM | 300 | OG | SER | B | 239 | 51.641 | 52.269 | 83.617 | 1.00 | 18.45 | 8 |
| ATOM | 301 | N | TYR | B | 240 | 49.001 | 52.014 | 84.627 | 1.00 | 13.21 | 7 |
| ATOM | 302 | CA | TYR | B | 240 | 48.106 | 53.162 | 84.893 | 1.00 | 12.92 | 6 |
| ATOM | 303 | C | TYR | B | 240 | 48.606 | 53.768 | 86.193 | 1.00 | 16.77 | 6 |
| ATOM | 304 | O | TYR | B | 240 | 49.296 | 53.112 | 86.967 | 1.00 | 13.76 | 8 |
| ATOM | 305 | CB | TYR | B | 240 | 46.625 | 52.779 | 85.049 | 1.00 | 14.15 | 6 |
| ATOM | 306 | CG | TYR | B | 240 | 45.961 | 52.350 | 83.771 | 1.00 | 14.05 | 6 |
| ATOM | 307 | CD1 | TYR | B | 240 | 46.029 | 51.017 | 83.369 | 1.00 | 14.29 | 6 |
| ATOM | 308 | CD2 | TYR | B | 240 | 45.257 | 53.247 | 82.968 | 1.00 | 13.70 | 6 |
| ATOM | 309 | CE1 | TYR | B | 240 | 45.411 | 50.589 | 82.200 | 1.00 | 13.77 | 6 |
| ATOM | 310 | CE2 | TYR | B | 240 | 44.636 | 52.828 | 81.798 | 1.00 | 13.45 | 6 |
| ATOM | 311 | CZ | TYR | B | 240 | 44.717 | 51.500 | 81.422 | 1.00 | 12.96 | 6 |
| ATOM | 312 | OH | TYR | B | 240 | 44.103 | 51.084 | 80.270 | 1.00 | 15.08 | 8 |
| ATOM | 313 | N | ARG | B | 241 | 48.242 | 55.025 | 86.405 | 1.00 | 20.46 | 7 |
| ATOM | 314 | CA | ARG | B | 241 | 48.645 | 55.757 | 87.590 | 1.00 | 23.22 | 6 |
| ATOM | 315 | C | ARG | B | 241 | 47.620 | 56.851 | 87.864 | 1.00 | 24.40 | 6 |
| ATOM | 316 | O | ARG | B | 241 | 47.245 | 57.612 | 86.963 | 1.00 | 24.95 | 8 |
| ATOM | 317 | CB | ARG | B | 241 | 50.041 | 56.348 | 87.374 | 1.00 | 26.13 | 6 |
| ATOM | 318 | CG | ARG | B | 241 | 50.506 | 57.292 | 88.455 | 1.00 | 29.67 | 6 |
| ATOM | 319 | CD | ARG | B | 241 | 51.934 | 57.758 | 88.217 | 1.00 | 33.23 | 6 |
| ATOM | 320 | NE | ARG | B | 241 | 52.921 | 56.783 | 88.675 | 1.00 | 34.47 | 7 |
| ATOM | 321 | CZ | ARG | B | 241 | 54.224 | 57.031 | 88.773 | 1.00 | 35.55 | 6 |
| ATOM | 322 | NH1 | ARG | B | 241 | 54.705 | 58.220 | 88.439 | 1.00 | 35.95 | 7 |
| ATOM | 323 | NH2 | ARG | B | 241 | 55.042 | 56.100 | 89.213 | 1.00 | 37.97 | 7 |
| ATOM | 324 | N | LYS | B | 242 | 47.171 | 56.931 | 89.108 | 1.00 | 27.52 | 7 |
| ATOM | 325 | CA | LYS | B | 242 | 46.194 | 57.936 | 89.484 | 1.00 | 30.61 | 6 |
| ATOM | 326 | C | LYS | B | 242 | 46.335 | 58.174 | 90.961 | 1.00 | 30.89 | 6 |
| ATOM | 327 | O | LYS | B | 242 | 46.571 | 57.251 | 91.748 | 1.00 | 28.07 | 8 |
| ATOM | 328 | CB | LYS | B | 242 | 44.778 | 57.481 | 89.129 | 1.00 | 34.16 | 6 |
| ATOM | 329 | CG | LYS | B | 242 | 43.677 | 58.118 | 89.967 | 1.00 | 35.51 | 6 |
| ATOM | 330 | CD | LYS | B | 242 | 42.310 | 57.582 | 89.566 | 1.00 | 37.84 | 6 |
| ATOM | 331 | CE | LYS | B | 242 | 41.204 | 58.155 | 90.435 | 1.00 | 37.75 | 6 |
| ATOM | 332 | NZ | LYS | B | 242 | 39.878 | 57.578 | 90.081 | 1.00 | 38.94 | 7 |
| ATOM | 333 | N | GLN | B | 243 | 46.183 | 59.400 | 91.347 | 1.00 | 32.69 | 7 |
| ATOM | 334 | CA | GLN | B | 243 | 46.279 | 59.730 | 92.715 | 1.00 | 34.93 | 6 |
| ATOM | 335 | C | GLN | B | 243 | 44.880 | 59.812 | 93.251 | 1.00 | 34.91 | 6 |
| ATOM | 336 | O | GLN | B | 243 | 43.957 | 60.271 | 92.572 | 1.00 | 35.97 | 8 |
| ATOM | 337 | CB | GLN | B | 243 | 47.062 | 61.026 | 92.846 | 1.00 | 34.09 | 6 |
| ATOM | 338 | CG | GLN | B | 243 | 47.113 | 61.600 | 94.240 | 1.00 | 37.09 | 6 |
| ATOM | 339 | CD | GLN | B | 243 | 47.566 | 63.031 | 94.182 | 1.00 | 39.39 | 6 |
| ATOM | 340 | OE1 | GLN | B | 243 | 48.654 | 63.322 | 93.681 | 1.00 | 40.63 | 8 |
| ATOM | 341 | NE2 | GLN | B | 243 | 46.909 | 64.093 | 94.647 | 1.00 | 38.39 | 7 |
| ATOM | 342 | N | VAL | B | 244 | 44.718 | 59.373 | 94.468 | 1.00 | 36.59 | 7 |
| ATOM | 343 | CA | VAL | B | 244 | 43.406 | 59.393 | 95.009 | 1.00 | 37.18 | 6 |
| ATOM | 344 | C | VAL | B | 244 | 43.468 | 59.384 | 96.510 | 1.00 | 37.74 | 6 |
| ATOM | 345 | O | VAL | B | 244 | 44.535 | 59.262 | 97.102 | 1.00 | 37.52 | 8 |
| ATOM | 346 | CB | VAL | B | 244 | 42.616 | 58.214 | 94.435 | 1.00 | 36.95 | 6 |
| ATOM | 347 | CG1 | VAL | B | 244 | 43.233 | 56.906 | 94.890 | 1.00 | 36.04 | 6 |
| ATOM | 348 | CG2 | VAL | B | 244 | 41.155 | 58.302 | 94.859 | 1.00 | 38.56 | 6 |
| ATOM | 349 | N | VAL | B | 245 | 42.297 | 59.549 | 97.067 | 1.00 | 38.33 | 7 |
| ATOM | 350 | CA | VAL | B | 245 | 42.255 | 59.621 | 98.489 | 1.00 | 39.76 | 6 |
| ATOM | 351 | C | VAL | B | 245 | 41.666 | 58.402 | 99.084 | 1.00 | 39.05 | 6 |
| ATOM | 352 | O | VAL | B | 245 | 40.656 | 57.879 | 98.609 | 1.00 | 39.51 | 8 |
| ATOM | 353 | CB | VAL | B | 245 | 41.457 | 60.864 | 98.957 | 1.00 | 41.36 | 6 |
| ATOM | 354 | CG1 | VAL | B | 245 | 42.155 | 62.143 | 98.540 | 1.00 | 42.45 | 6 |
| ATOM | 355 | CG2 | VAL | B | 245 | 40.048 | 60.833 | 98.405 | 1.00 | 41.58 | 6 |
| ATOM | 356 | N | ILE | B | 246 | 42.306 | 57.977 | 100.102 | 1.00 | 38.11 | 7 |
| ATOM | 357 | CA | ILE | B | 246 | 41.716 | 56.887 | 100.765 | 1.00 | 36.88 | 6 |
| ATOM | 358 | C | ILE | B | 246 | 41.337 | 57.441 | 102.116 | 1.00 | 37.71 | 6 |
| ATOM | 359 | O | ILE | B | 246 | 41.021 | 58.623 | 102.232 | 1.00 | 37.36 | 8 |
| ATOM | 360 | CB | ILE | B | 246 | 42.671 | 55.676 | 100.801 | 1.00 | 35.21 | 6 |
| ATOM | 361 | CG1 | ILE | B | 246 | 43.487 | 55.607 | 99.503 | 1.00 | 33.18 | 6 |
| ATOM | 362 | CG2 | ILE | B | 246 | 41.899 | 54.373 | 101.002 | 1.00 | 33.95 | 6 |
| ATOM | 363 | CD1 | ILE | B | 246 | 43.655 | 54.207 | 98.965 | 1.00 | 30.37 | 6 |
| ATOM | 364 | N | ASP | B | 247 | 41.379 | 56.618 | 103.113 | 1.00 | 39.08 | 7 |
| ATOM | 365 | CA | ASP | B | 247 | 41.026 | 57.135 | 104.414 | 1.00 | 41.19 | 6 |
| ATOM | 366 | C | ASP | B | 247 | 41.469 | 58.548 | 104.601 | 1.00 | 42.67 | 6 |
| ATOM | 367 | O | ASP | B | 247 | 40.801 | 59.325 | 105.294 | 1.00 | 42.85 | 8 |
| ATOM | 368 | CB | ASP | B | 247 | 41.542 | 56.191 | 105.454 | 1.00 | 39.66 | 6 |
| ATOM | 369 | CG | ASP | B | 247 | 40.996 | 54.849 | 105.034 | 1.00 | 39.63 | 6 |
| ATOM | 370 | OD1 | ASP | B | 247 | 39.784 | 54.754 | 104.830 | 1.00 | 40.80 | 8 |
| ATOM | 371 | OD2 | ASP | B | 247 | 41.776 | 53.879 | 104.907 | 1.00 | 40.43 | 8 |
| ATOM | 372 | N | GLY | B | 248 | 42.551 | 58.914 | 104.020 | 1.00 | 44.34 | 7 |
| ATOM | 373 | CA | GLY | B | 248 | 42.909 | 60.269 | 104.265 | 1.00 | 46.95 | 6 |
| ATOM | 374 | C | GLY | B | 248 | 44.380 | 60.455 | 104.122 | 1.00 | 47.75 | 6 |
| ATOM | 375 | O | GLY | B | 248 | 44.992 | 61.284 | 104.786 | 1.00 | 48.28 | 8 |
| ATOM | 376 | N | GLU | B | 249 | 44.951 | 59.682 | 103.217 | 1.00 | 49.39 | 7 |
| ATOM | 377 | CA | GLU | B | 249 | 46.376 | 59.722 | 102.912 | 1.00 | 50.74 | 6 |
| ATOM | 378 | C | GLU | B | 249 | 46.604 | 60.068 | 101.431 | 1.00 | 50.53 | 6 |
| ATOM | 379 | O | GLU | B | 249 | 47.463 | 59.438 | 100.813 | 1.00 | 50.71 | 8 |
| ATOM | 380 | CB | GLU | B | 249 | 47.005 | 58.321 | 103.151 | 1.00 | 52.59 | 6 |
| ATOM | 381 | CG | GLU | B | 249 | 46.708 | 57.275 | 102.054 | 1.00 | 58.27 | 6 |
| ATOM | 382 | CD | GLU | B | 249 | 46.776 | 55.798 | 102.452 | 1.00 | 60.54 | 6 |
| ATOM | 383 | OE1 | GLU | B | 249 | 46.023 | 55.416 | 103.375 | 1.00 | 61.57 | 8 |
| ATOM | 384 | OE2 | GLU | B | 249 | 47.566 | 55.042 | 101.858 | 1.00 | 62.66 | 8 |
| ATOM | 385 | N | THR | B | 250 | 45.896 | 61.021 | 100.823 | 1.00 | 49.93 | 7 |
| ATOM | 386 | CA | THR | B | 250 | 46.167 | 61.135 | 99.370 | 1.00 | 46.91 | 6 |
| ATOM | 387 | C | THR | B | 250 | 47.385 | 60.282 | 99.090 | 1.00 | 44.79 | 6 |
| ATOM | 388 | O | THR | B | 250 | 48.403 | 60.423 | 99.746 | 1.00 | 43.24 | 8 |
| ATOM | 389 | CB | THR | B | 250 | 46.410 | 62.567 | 98.871 | 1.00 | 48.41 | 6 |
| ATOM | 390 | OG1 | THR | B | 250 | 45.225 | 63.364 | 99.052 | 1.00 | 47.77 | 8 |
| ATOM | 391 | CG2 | THR | B | 250 | 46.810 | 62.543 | 97.406 | 1.00 | 49.37 | 6 |
| ATOM | 392 | N | CYS | B | 251 | 47.263 | 59.400 | 98.104 | 1.00 | 42.00 | 7 |
| ATOM | 393 | CA | CYS | B | 251 | 48.340 | 58.493 | 97.739 | 1.00 | 38.38 | 6 |
| ATOM | 394 | C | CYS | B | 251 | 48.362 | 58.246 | 96.231 | 1.00 | 35.09 | 6 |
| ATOM | 395 | O | CYS | B | 251 | 47.395 | 58.508 | 95.525 | 1.00 | 35.14 | 8 |
| ATOM | 396 | CB | CYS | B | 251 | 48.206 | 57.174 | 98.501 | 1.00 | 38.73 | 6 |
| ATOM | 397 | SG | CYS | B | 251 | 46.508 | 56.535 | 98.621 | 1.00 | 36.78 | 16 |
| ATOM | 398 | N | LEU | B | 252 | 49.484 | 57.734 | 95.740 | 1.00 | 31.83 | 7 |
| ATOM | 399 | CA | LEU | B | 252 | 49.646 | 57.458 | 94.322 | 1.00 | 32.24 | 6 |
| ATOM | 400 | C | LEU | B | 252 | 49.388 | 55.986 | 94.025 | 1.00 | 31.25 | 6 |
| ATOM | 401 | O | LEU | B | 252 | 50.054 | 55.110 | 94.580 | 1.00 | 30.93 | 8 |
| ATOM | 402 | CB | LEU | B | 252 | 51.064 | 57.819 | 93.876 | 1.00 | 34.14 | 6 |
| ATOM | 403 | CG | LEU | B | 252 | 51.216 | 58.582 | 92.561 | 1.00 | 34.96 | 6 |
| ATOM | 404 | CD1 | LEU | B | 252 | 51.493 | 57.621 | 91.409 | 1.00 | 36.92 | 6 |
| ATOM | 405 | CD2 | LEU | B | 252 | 49.983 | 59.431 | 92.281 | 1.00 | 33.48 | 6 |
| ATOM | 406 | N | LEU | B | 253 | 48.426 | 55.718 | 93.147 | 1.00 | 27.51 | 7 |
| ATOM | 407 | CA | LEU | B | 253 | 48.108 | 54.343 | 92.781 | 1.00 | 25.51 | 6 |
| ATOM | 408 | C | LEU | B | 253 | 48.688 | 53.947 | 91.432 | 1.00 | 23.87 | 6 |
| ATOM | 409 | O | LEU | B | 253 | 48.318 | 54.505 | 90.398 | 1.00 | 21.99 | 8 |
| ATOM | 410 | CB | LEU | B | 253 | 46.591 | 54.104 | 92.763 | 1.00 | 24.50 | 6 |
| ATOM | 411 | CG | LEU | B | 253 | 45.777 | 54.192 | 94.058 | 1.00 | 24.94 | 6 |
| ATOM | 412 | CD1 | LEU | B | 253 | 44.410 | 53.570 | 93.822 | 1.00 | 23.95 | 6 |
| ATOM | 413 | CD2 | LEU | B | 253 | 46.485 | 53.468 | 95.191 | 1.00 | 24.50 | 6 |
| ATOM | 414 | N | ASP | B | 254 | 49.642 | 53.023 | 91.457 | 1.00 | 23.18 | 7 |
| ATOM | 415 | CA | ASP | B | 254 | 50.248 | 52.506 | 90.234 | 1.00 | 22.76 | 6 |
| ATOM | 416 | C | ASP | B | 254 | 49.555 | 51.176 | 89.984 | 1.00 | 22.41 | 6 |
| ATOM | 417 | O | ASP | B | 254 | 49.613 | 50.275 | 90.826 | 1.00 | 23.13 | 8 |
| ATOM | 418 | CB | ASP | B | 254 | 51.753 | 52.279 | 90.413 | 1.00 | 27.20 | 6 |
| ATOM | 419 | CG | ASP | B | 254 | 52.553 | 53.572 | 90.386 | 1.00 | 29.86 | 6 |
| ATOM | 420 | OD1 | ASP | B | 254 | 51.968 | 54.662 | 90.559 | 1.00 | 31.54 | 8 |
| ATOM | 421 | OD2 | ASP | B | 254 | 53.783 | 53.490 | 90.187 | 1.00 | 32.40 | 8 |
| ATOM | 422 | N | ILE | B | 255 | 48.864 | 51.068 | 88.855 | 1.00 | 18.90 | 7 |
| ATOM | 423 | CA | ILE | B | 255 | 48.149 | 49.841 | 88.534 | 1.00 | 15.08 | 6 |
| ATOM | 424 | C | ILE | B | 255 | 48.683 | 49.146 | 87.298 | 1.00 | 12.73 | 6 |
| ATOM | 425 | O | ILE | B | 255 | 48.663 | 49.707 | 86.203 | 1.00 | 13.38 | 8 |
| ATOM | 426 | CB | ILE | B | 255 | 46.643 | 50.099 | 88.331 | 1.00 | 16.48 | 6 |
| ATOM | 427 | CG1 | ILE | B | 255 | 46.047 | 50.763 | 89.578 | 1.00 | 18.10 | 6 |
| ATOM | 428 | CG2 | ILE | B | 255 | 45.929 | 48.788 | 88.033 | 1.00 | 14.72 | 6 |
| ATOM | 429 | CD1 | ILE | B | 255 | 44.552 | 51.013 | 89.495 | 1.00 | 21.51 | 6 |
| ATOM | 430 | N | LEU | B | 256 | 49.165 | 47.920 | 87.482 | 1.00 | 9.73 | 7 |
| ATOM | 431 | CA | LEU | B | 256 | 49.682 | 47.131 | 86.375 | 1.00 | 8.12 | 6 |
| ATOM | 432 | C | LEU | B | 256 | 48.586 | 46.164 | 85.930 | 1.00 | 7.21 | 6 |
| ATOM | 433 | O | LEU | B | 256 | 48.192 | 45.266 | 86.674 | 1.00 | 6.29 | 8 |
| ATOM | 434 | CB | LEU | B | 256 | 50.936 | 46.356 | 86.791 | 1.00 | 6.84 | 6 |
| ATOM | 435 | CG | LEU | B | 256 | 51.690 | 45.635 | 85.670 | 1.00 | 4.43 | 6 |
| ATOM | 436 | CD1 | LEU | B | 256 | 52.229 | 46.653 | 84.682 | 1.00 | 6.66 | 6 |
| ATOM | 437 | CD2 | LEU | B | 256 | 52.819 | 44.814 | 86.242 | 1.00 | 2.53 | 6 |
| ATOM | 438 | N | ASP | B | 257 | 48.052 | 46.412 | 84.738 | 1.00 | 7.21 | 7 |
| ATOM | 439 | CA | ASP | B | 257 | 46.998 | 45.594 | 84.145 | 1.00 | 7.45 | 6 |
| ATOM | 440 | C | ASP | B | 257 | 47.696 | 44.548 | 83.275 | 1.00 | 8.22 | 6 |
| ATOM | 441 | O | ASP | B | 257 | 48.224 | 44.860 | 82.205 | 1.00 | 8.74 | 8 |
| ATOM | 442 | CB | ASP | B | 257 | 46.073 | 46.504 | 83.329 | 1.00 | 8.70 | 6 |
| ATOM | 443 | CG | ASP | B | 257 | 44.950 | 45.753 | 82.657 | 1.00 | 9.50 | 6 |
| ATOM | 444 | OD1 | ASP | B | 257 | 44.511 | 44.702 | 83.173 | 1.00 | 6.85 | 8 |
| ATOM | 445 | OD2 | ASP | B | 257 | 44.508 | 46.236 | 81.596 | 1.00 | 10.23 | 8 |
| ATOM | 446 | N | THR | B | 258 | 47.699 | 43.308 | 83.756 | 1.00 | 10.11 | 7 |
| ATOM | 447 | CA | THR | B | 258 | 48.418 | 42.229 | 83.092 | 1.00 | 8.35 | 6 |
| ATOM | 448 | C | THR | B | 258 | 47.582 | 41.437 | 82.091 | 1.00 | 8.23 | 6 |
| ATOM | 449 | O | THR | B | 258 | 46.371 | 41.621 | 81.967 | 1.00 | 11.99 | 8 |
| ATOM | 450 | CB | THR | B | 258 | 48.989 | 41.243 | 84.142 | 1.00 | 8.13 | 6 |
| ATOM | 451 | OG1 | THR | B | 258 | 47.908 | 40.502 | 84.744 | 1.00 | 6.51 | 8 |
| ATOM | 452 | CG2 | THR | B | 258 | 49.759 | 42.007 | 85.204 | 1.00 | 2.22 | 6 |
| ATOM | 453 | N | ALA | B | 259 | 48.285 | 40.557 | 81.385 | 1.00 | 5.52 | 7 |
| ATOM | 454 | CA | ALA | B | 259 | 47.688 | 39.713 | 80.384 | 1.00 | 7.48 | 6 |
| ATOM | 455 | C | ALA | B | 259 | 47.105 | 38.476 | 80.990 | 1.00 | 9.19 | 6 |
| ATOM | 456 | O | ALA | B | 259 | 47.532 | 38.035 | 82.052 | 1.00 | 11.31 | 8 |
| ATOM | 457 | CB | ALA | B | 259 | 48.699 | 39.337 | 79.312 | 1.00 | 6.66 | 6 |
| ATOM | 458 | N | GLY | B | 260 | 46.125 | 37.902 | 80.346 | 1.00 | 9.72 | 7 |
| ATOM | 459 | CA | GLY | B | 260 | 45.586 | 36.662 | 80.807 | 1.00 | 11.00 | 6 |
| ATOM | 460 | C | GLY | B | 260 | 46.120 | 35.608 | 79.867 | 1.00 | 12.53 | 6 |
| ATOM | 461 | O | GLY | B | 260 | 46.256 | 34.439 | 80.207 | 1.00 | 13.43 | 8 |
| ATOM | 462 | N | GLN | B | 261 | 46.430 | 36.074 | 78.644 | 1.00 | 12.40 | 7 |
| ATOM | 463 | CA | GLN | B | 261 | 46.917 | 35.237 | 77.522 | 1.00 | 15.05 | 6 |
| ATOM | 464 | C | GLN | B | 261 | 48.180 | 34.429 | 77.861 | 1.00 | 15.70 | 6 |
| ATOM | 465 | O | GLN | B | 261 | 49.300 | 34.940 | 77.745 | 1.00 | 16.74 | 8 |
| ATOM | 466 | CB | GLN | B | 261 | 47.116 | 36.116 | 76.258 | 1.00 | 17.11 | 6 |
| ATOM | 467 | CG | GLN | B | 261 | 45.954 | 36.071 | 75.220 | 1.00 | 15.52 | 6 |
| ATOM | 468 | CD | GLN | B | 261 | 46.201 | 36.835 | 73.916 | 1.00 | 16.27 | 6 |
| ATOM | 469 | OE1 | GLN | B | 261 | 46.262 | 36.235 | 72.832 | 1.00 | 16.77 | 8 |
| ATOM | 470 | NE2 | GLN | B | 261 | 46.386 | 38.148 | 73.795 | 1.00 | 10.58 | 7 |
| ATOM | 471 | N | GLU | B | 262 | 47.959 | 33.140 | 78.307 | 1.00 | 15.37 | 7 |
| ATOM | 472 | CA | GLU | B | 262 | 49.081 | 32.279 | 78.657 | 1.00 | 18.10 | 6 |
| ATOM | 473 | C | GLU | B | 262 | 50.305 | 32.595 | 77.803 | 1.00 | 17.51 | 6 |
| ATOM | 474 | O | GLU | B | 262 | 51.428 | 32.313 | 78.142 | 1.00 | 18.72 | 8 |
| ATOM | 475 | CB | GLU | B | 262 | 48.692 | 30.807 | 78.513 | 1.00 | 19.14 | 6 |
| ATOM | 476 | CG | GLU | B | 262 | 47.535 | 30.383 | 79.401 | 1.00 | 24.07 | 6 |
| ATOM | 477 | CD | GLU | B | 262 | 47.995 | 29.842 | 80.741 | 1.00 | 25.10 | 6 |
| ATOM | 478 | OE1 | GLU | B | 262 | 49.221 | 29.807 | 80.977 | 1.00 | 23.90 | 8 |
| ATOM | 479 | OE2 | GLU | B | 262 | 47.130 | 29.454 | 81.554 | 1.00 | 28.42 | 8 |
| ATOM | 480 | N | GLU | B | 263 | 50.012 | 33.249 | 76.671 | 1.00 | 18.44 | 7 |
| ATOM | 481 | CA | GLU | B | 263 | 51.039 | 33.652 | 75.719 | 1.00 | 22.43 | 6 |
| ATOM | 482 | C | GLU | B | 263 | 52.074 | 34.558 | 76.376 | 1.00 | 23.64 | 6 |
| ATOM | 483 | O | GLU | B | 263 | 53.238 | 34.522 | 75.992 | 1.00 | 23.59 | 8 |
| ATOM | 484 | CB | GLU | B | 263 | 50.409 | 34.349 | 74.512 | 1.00 | 21.78 | 6 |
| ATOM | 485 | CG | GLU | B | 263 | 51.375 | 34.601 | 73.365 | 1.00 | 25.68 | 6 |
| ATOM | 486 | CD | GLU | B | 263 | 50.673 | 35.064 | 72.103 | 1.00 | 28.86 | 6 |
| ATOM | 487 | OE1 | GLU | B | 263 | 49.432 | 35.202 | 72.130 | 1.00 | 25.25 | 8 |
| ATOM | 488 | OE2 | GLU | B | 263 | 51.364 | 35.287 | 71.087 | 1.00 | 31.70 | 8 |
| ATOM | 489 | N | TYR | B | 264 | 51.656 | 35.348 | 77.330 | 1.00 | 25.75 | 7 |
| ATOM | 490 | CA | TYR | B | 264 | 52.595 | 36.244 | 77.976 | 1.00 | 28.14 | 6 |
| ATOM | 491 | C | TYR | B | 264 | 52.795 | 35.748 | 79.380 | 1.00 | 29.31 | 6 |
| ATOM | 492 | O | TYR | B | 264 | 52.692 | 36.465 | 80.375 | 1.00 | 32.38 | 8 |
| ATOM | 493 | CB | TYR | B | 264 | 52.060 | 37.679 | 77.965 | 1.00 | 26.09 | 6 |
| ATOM | 494 | CG | TYR | B | 264 | 51.618 | 38.064 | 76.572 | 1.00 | 25.37 | 6 |
| ATOM | 495 | CD1 | TYR | B | 264 | 52.548 | 38.385 | 75.587 | 1.00 | 24.51 | 6 |
| ATOM | 496 | CD2 | TYR | B | 264 | 50.269 | 38.057 | 76.228 | 1.00 | 27.35 | 6 |
| ATOM | 497 | CE1 | TYR | B | 264 | 52.142 | 38.682 | 74.292 | 1.00 | 26.47 | 6 |
| ATOM | 498 | CE2 | TYR | B | 264 | 49.857 | 38.353 | 74.939 | 1.00 | 27.67 | 6 |
| ATOM | 499 | CZ | TYR | B | 264 | 50.796 | 38.662 | 73.976 | 1.00 | 25.73 | 6 |
| ATOM | 500 | OH | TYR | B | 264 | 50.385 | 38.936 | 72.694 | 1.00 | 30.28 | 8 |
| ATOM | 501 | N | SER | B | 265 | 53.085 | 34.472 | 79.430 | 1.00 | 29.70 | 7 |
| ATOM | 502 | CA | SER | B | 265 | 53.271 | 33.818 | 80.697 | 1.00 | 29.71 | 6 |
| ATOM | 503 | C | SER | B | 265 | 54.618 | 34.108 | 81.316 | 1.00 | 30.48 | 6 |
| ATOM | 504 | O | SER | B | 265 | 54.808 | 33.947 | 82.531 | 1.00 | 30.50 | 8 |
| ATOM | 505 | CB | SER | B | 265 | 53.063 | 32.305 | 80.512 | 1.00 | 28.92 | 6 |
| ATOM | 506 | OG | SER | B | 265 | 52.070 | 32.030 | 79.530 | 1.00 | 30.09 | 8 |
| ATOM | 507 | N | ALA | B | 266 | 55.548 | 34.524 | 80.484 | 1.00 | 31.11 | 7 |
| ATOM | 508 | CA | ALA | B | 266 | 56.875 | 34.811 | 80.991 | 1.00 | 30.12 | 6 |
| ATOM | 509 | C | ALA | B | 266 | 57.068 | 36.260 | 81.413 | 1.00 | 29.72 | 6 |
| ATOM | 510 | O | ALA | B | 266 | 58.191 | 36.728 | 81.417 | 1.00 | 31.16 | 8 |
| ATOM | 511 | CB | ALA | B | 266 | 57.925 | 34.425 | 79.963 | 1.00 | 31.38 | 6 |
| ATOM | 512 | N | MET | B | 267 | 56.041 | 36.985 | 81.751 | 1.00 | 27.80 | 7 |
| ATOM | 513 | CA | MET | B | 267 | 56.268 | 38.359 | 82.176 | 1.00 | 24.41 | 6 |
| ATOM | 514 | C | MET | B | 267 | 55.636 | 38.516 | 83.520 | 1.00 | 22.53 | 6 |
| ATOM | 515 | O | MET | B | 267 | 55.717 | 39.543 | 84.187 | 1.00 | 20.93 | 8 |
| ATOM | 516 | CB | MET | B | 267 | 55.643 | 39.371 | 81.221 | 1.00 | 25.81 | 6 |
| ATOM | 517 | CG | MET | B | 267 | 55.983 | 39.137 | 79.776 | 1.00 | 27.94 | 6 |
| ATOM | 518 | SD | MET | B | 267 | 54.796 | 39.878 | 78.638 | 1.00 | 29.01 | 16 |
| ATOM | 519 | CE | MET | B | 267 | 55.268 | 41.606 | 78.718 | 1.00 | 30.75 | 6 |
| ATOM | 520 | N | ARG | B | 268 | 54.973 | 37.417 | 83.859 | 1.00 | 20.74 | 7 |
| ATOM | 521 | CA | ARG | B | 268 | 54.300 | 37.353 | 85.113 | 1.00 | 21.74 | 6 |
| ATOM | 522 | C | ARG | B | 268 | 55.300 | 37.447 | 86.220 | 1.00 | 23.40 | 6 |
| ATOM | 523 | O | ARG | B | 268 | 55.081 | 38.106 | 87.245 | 1.00 | 24.31 | 8 |
| ATOM | 524 | CB | ARG | B | 268 | 53.535 | 36.063 | 85.299 | 1.00 | 21.08 | 6 |
| ATOM | 525 | CG | ARG | B | 268 | 53.105 | 35.395 | 84.017 | 1.00 | 22.29 | 6 |
| ATOM | 526 | CD | ARG | B | 268 | 52.081 | 34.355 | 84.395 | 1.00 | 25.37 | 6 |
| ATOM | 527 | NE | ARG | B | 268 | 50.994 | 34.346 | 83.457 | 1.00 | 26.23 | 7 |
| ATOM | 528 | CZ | ARG | B | 268 | 50.397 | 33.229 | 83.149 | 1.00 | 27.97 | 6 |
| ATOM | 529 | NH1 | ARG | B | 268 | 50.775 | 32.098 | 83.737 | 1.00 | 25.98 | 7 |
| ATOM | 530 | NH2 | ARG | B | 268 | 49.426 | 33.207 | 82.246 | 1.00 | 23.99 | 7 |
| ATOM | 531 | N | ASP | B | 269 | 56.425 | 36.765 | 86.003 | 1.00 | 25.00 | 7 |
| ATOM | 532 | CA | ASP | B | 269 | 57.494 | 36.673 | 86.993 | 1.00 | 28.24 | 6 |
| ATOM | 533 | C | ASP | B | 269 | 57.922 | 38.039 | 87.590 | 1.00 | 27.60 | 6 |
| ATOM | 534 | O | ASP | B | 269 | 57.868 | 38.206 | 88.812 | 1.00 | 26.41 | 8 |
| ATOM | 535 | CB | ASP | B | 269 | 58.618 | 35.819 | 86.438 | 1.00 | 31.92 | 6 |
| ATOM | 536 | CG | ASP | B | 269 | 58.386 | 34.349 | 86.748 | 1.00 | 34.91 | 6 |
| ATOM | 537 | OD1 | ASP | B | 269 | 57.290 | 33.828 | 86.438 | 1.00 | 38.62 | 8 |
| ATOM | 538 | OD2 | ASP | B | 269 | 59.307 | 33.714 | 87.290 | 1.00 | 38.97 | 8 |
| ATOM | 539 | N | GLN | B | 270 | 58.346 | 39.010 | 86.785 | 1.00 | 27.67 | 7 |
| ATOM | 540 | CA | GLN | B | 270 | 58.742 | 40.314 | 87.336 | 1.00 | 26.52 | 6 |
| ATOM | 541 | C | GLN | B | 270 | 57.555 | 41.086 | 87.963 | 1.00 | 24.96 | 6 |
| ATOM | 542 | O | GLN | B | 270 | 57.655 | 41.578 | 89.089 | 1.00 | 23.72 | 8 |
| ATOM | 543 | CB | GLN | B | 270 | 59.449 | 41.190 | 86.246 | 1.00 | 31.64 | 6 |
| ATOM | 544 | CG | GLN | B | 270 | 60.815 | 41.761 | 86.654 | 1.00 | 39.68 | 6 |
| ATOM | 545 | CD | GLN | B | 270 | 61.347 | 42.954 | 85.831 | 1.00 | 44.31 | 6 |
| ATOM | 546 | OE1 | GLN | B | 270 | 61.003 | 43.145 | 84.660 | 1.00 | 45.91 | 8 |
| ATOM | 547 | NE2 | GLN | B | 270 | 62.211 | 43.876 | 86.256 | 1.00 | 45.94 | 7 |
| ATOM | 548 | N | TYR | B | 271 | 56.465 | 41.182 | 87.225 | 1.00 | 21.40 | 7 |
| ATOM | 549 | CA | TYR | B | 271 | 55.347 | 41.931 | 87.757 | 1.00 | 17.72 | 6 |
| ATOM | 550 | C | TYR | B | 271 | 54.986 | 41.351 | 89.098 | 1.00 | 16.86 | 6 |
| ATOM | 551 | O | TYR | B | 271 | 54.292 | 41.965 | 89.917 | 1.00 | 16.16 | 8 |
| ATOM | 552 | CB | TYR | B | 271 | 54.172 | 41.995 | 86.727 | 1.00 | 13.67 | 6 |
| ATOM | 553 | CG | TYR | B | 271 | 53.196 | 40.835 | 86.401 | 1.00 | 13.98 | 6 |
| ATOM | 554 | CD1 | TYR | B | 271 | 52.302 | 40.312 | 87.337 | 1.00 | 17.13 | 6 |
| ATOM | 555 | CD2 | TYR | B | 271 | 53.180 | 40.306 | 85.110 | 1.00 | 13.22 | 6 |
| ATOM | 556 | CE1 | TYR | B | 271 | 51.420 | 39.294 | 86.993 | 1.00 | 16.95 | 6 |
| ATOM | 557 | CE2 | TYR | B | 271 | 52.297 | 39.319 | 84.754 | 1.00 | 12.52 | 6 |
| ATOM | 558 | CZ | TYR | B | 271 | 51.421 | 38.821 | 85.701 | 1.00 | 14.39 | 6 |
| ATOM | 559 | OH | TYR | B | 271 | 50.537 | 37.830 | 85.356 | 1.00 | 16.88 | 8 |
| ATOM | 560 | N | MET | B | 272 | 55.457 | 40.159 | 89.336 | 1.00 | 15.53 | 7 |
| ATOM | 561 | CA | MET | B | 272 | 55.146 | 39.625 | 90.628 | 1.00 | 16.73 | 6 |
| ATOM | 562 | C | MET | B | 272 | 56.218 | 40.055 | 91.575 | 1.00 | 19.26 | 6 |
| ATOM | 563 | O | MET | B | 272 | 55.950 | 40.366 | 92.725 | 1.00 | 21.54 | 8 |
| ATOM | 564 | CB | MET | B | 272 | 55.037 | 38.105 | 90.604 | 1.00 | 17.85 | 6 |
| ATOM | 565 | CG | MET | B | 272 | 53.688 | 37.606 | 90.161 | 1.00 | 16.55 | 6 |
| ATOM | 566 | SD | MET | B | 272 | 53.775 | 35.984 | 89.392 | 1.00 | 14.21 | 16 |
| ATOM | 567 | CE | MET | B | 272 | 52.131 | 35.842 | 88.709 | 1.00 | 16.80 | 6 |
| ATOM | 568 | N | ARG | B | 273 | 57.436 | 40.089 | 91.072 | 1.00 | 21.00 | 7 |
| ATOM | 569 | CA | ARG | B | 273 | 58.417 | 40.492 | 92.006 | 1.00 | 21.44 | 6 |
| ATOM | 570 | C | ARG | B | 273 | 58.091 | 41.836 | 92.536 | 1.00 | 20.10 | 6 |
| ATOM | 571 | O | ARG | B | 273 | 57.993 | 42.042 | 93.741 | 1.00 | 19.92 | 8 |
| ATOM | 572 | CB | ARG | B | 273 | 59.813 | 40.482 | 91.416 | 1.00 | 23.58 | 6 |
| ATOM | 573 | CG | ARG | B | 273 | 60.475 | 39.145 | 91.650 | 1.00 | 28.23 | 6 |
| ATOM | 574 | CD | ARG | B | 273 | 61.924 | 39.165 | 91.272 | 1.00 | 31.68 | 6 |
| ATOM | 575 | NE | ARG | B | 273 | 62.116 | 39.066 | 89.831 | 1.00 | 33.66 | 7 |
| ATOM | 576 | CZ | ARG | B | 273 | 62.268 | 37.918 | 89.176 | 1.00 | 37.50 | 6 |
| ATOM | 577 | NH1 | ARG | B | 273 | 62.217 | 36.764 | 89.832 | 1.00 | 37.42 | 7 |
| ATOM | 578 | NH2 | ARG | B | 273 | 62.481 | 37.925 | 87.867 | 1.00 | 38.80 | 7 |
| ATOM | 579 | N | THR | B | 274 | 57.906 | 42.780 | 91.662 | 1.00 | 18.35 | 7 |
| ATOM | 580 | CA | THR | B | 274 | 57.749 | 44.117 | 92.113 | 1.00 | 20.36 | 6 |
| ATOM | 581 | C | THR | B | 274 | 56.394 | 44.512 | 92.708 | 1.00 | 18.73 | 6 |
| ATOM | 582 | O | THR | B | 274 | 56.323 | 45.441 | 93.518 | 1.00 | 19.21 | 8 |
| ATOM | 583 | CB | THR | B | 274 | 58.097 | 44.995 | 90.924 | 1.00 | 19.30 | 6 |
| ATOM | 584 | OG1 | THR | B | 274 | 57.173 | 44.775 | 89.852 | 1.00 | 24.51 | 8 |
| ATOM | 585 | CG2 | THR | B | 274 | 59.524 | 44.689 | 90.450 | 1.00 | 21.44 | 6 |
| ATOM | 586 | N | GLY | B | 275 | 55.330 | 43.823 | 92.330 | 1.00 | 16.89 | 7 |
| ATOM | 587 | CA | GLY | B | 275 | 53.993 | 44.150 | 92.803 | 1.00 | 14.72 | 6 |
| ATOM | 588 | C | GLY | B | 275 | 53.882 | 44.036 | 94.312 | 1.00 | 15.24 | 6 |
| ATOM | 589 | O | GLY | B | 275 | 54.337 | 43.066 | 94.918 | 1.00 | 16.13 | 8 |
| ATOM | 590 | N | GLU | B | 276 | 53.265 | 45.033 | 94.922 | 1.00 | 14.31 | 7 |
| ATOM | 591 | CA | GLU | B | 276 | 53.031 | 44.998 | 96.337 | 1.00 | 15.06 | 6 |
| ATOM | 592 | C | GLU | B | 276 | 51.622 | 44.500 | 96.558 | 1.00 | 16.32 | 6 |
| ATOM | 593 | O | GLU | B | 276 | 51.282 | 44.001 | 97.629 | 1.00 | 19.21 | 8 |
| ATOM | 594 | CB | GLU | B | 276 | 53.191 | 46.381 | 96.959 | 1.00 | 18.07 | 6 |
| ATOM | 595 | CG | GLU | B | 276 | 54.454 | 47.090 | 96.493 | 1.00 | 19.61 | 6 |
| ATOM | 596 | CD | GLU | B | 276 | 54.539 | 48.522 | 96.977 | 1.00 | 23.51 | 6 |
| ATOM | 597 | OE1 | GLU | B | 276 | 53.493 | 49.158 | 97.215 | 1.00 | 25.72 | 8 |
| ATOM | 598 | OE2 | GLU | B | 276 | 55.677 | 49.018 | 97.113 | 1.00 | 25.46 | 8 |
| ATOM | 599 | N | GLY | B | 277 | 50.787 | 44.655 | 95.529 | 1.00 | 16.19 | 7 |
| ATOM | 600 | CA | GLY | B | 277 | 49.381 | 44.251 | 95.623 | 1.00 | 13.63 | 6 |
| ATOM | 601 | C | GLY | B | 277 | 48.963 | 43.412 | 94.418 | 1.00 | 14.91 | 6 |
| ATOM | 602 | O | GLY | B | 277 | 49.495 | 43.577 | 93.334 | 1.00 | 14.02 | 8 |
| ATOM | 603 | N | PHE | B | 278 | 48.003 | 42.501 | 94.639 | 1.00 | 12.85 | 7 |
| ATOM | 604 | CA | PHE | B | 278 | 47.560 | 41.659 | 93.524 | 1.00 | 13.60 | 6 |
| ATOM | 605 | C | PHE | B | 278 | 46.102 | 41.389 | 93.558 | 1.00 | 12.89 | 6 |
| ATOM | 606 | O | PHE | B | 278 | 45.575 | 40.662 | 94.403 | 1.00 | 14.83 | 8 |
| ATOM | 607 | CB | PHE | B | 278 | 48.294 | 40.339 | 93.513 | 1.00 | 10.69 | 6 |
| ATOM | 608 | CG | PHE | B | 278 | 49.667 | 40.526 | 93.022 | 1.00 | 10.61 | 6 |
| ATOM | 609 | CD1 | PHE | B | 278 | 50.715 | 39.894 | 93.658 | 1.00 | 11.20 | 6 |
| ATOM | 610 | CD2 | PHE | B | 278 | 49.926 | 41.347 | 91.940 | 1.00 | 8.70 | 6 |
| ATOM | 611 | CE1 | PHE | B | 278 | 52.000 | 40.086 | 93.254 | 1.00 | 10.58 | 6 |
| ATOM | 612 | CE2 | PHE | B | 278 | 51.238 | 41.544 | 91.497 | 1.00 | 5.93 | 6 |
| ATOM | 613 | CZ | PHE | B | 278 | 52.276 | 40.914 | 92.169 | 1.00 | 7.62 | 6 |
| ATOM | 614 | N | LEU | B | 279 | 45.477 | 41.981 | 92.596 | 1.00 | 14.40 | 7 |
| ATOM | 615 | CA | LEU | B | 279 | 44.066 | 41.803 | 92.439 | 1.00 | 13.51 | 6 |
| ATOM | 616 | C | LEU | B | 279 | 43.810 | 40.540 | 91.679 | 1.00 | 12.64 | 6 |
| ATOM | 617 | O | LEU | B | 279 | 43.995 | 40.475 | 90.466 | 1.00 | 13.82 | 8 |
| ATOM | 618 | CB | LEU | B | 279 | 43.471 | 43.023 | 91.747 | 1.00 | 14.31 | 6 |
| ATOM | 619 | CG | LEU | B | 279 | 42.008 | 43.337 | 92.072 | 1.00 | 15.70 | 6 |
| ATOM | 620 | CD1 | LEU | B | 279 | 41.870 | 43.732 | 93.536 | 1.00 | 16.34 | 6 |
| ATOM | 621 | CD2 | LEU | B | 279 | 41.464 | 44.424 | 91.170 | 1.00 | 13.91 | 6 |
| ATOM | 622 | N | CYS | B | 280 | 43.376 | 39.513 | 92.389 | 1.00 | 11.95 | 7 |
| ATOM | 623 | CA | CYS | B | 280 | 43.085 | 38.230 | 91.729 | 1.00 | 9.99 | 6 |
| ATOM | 624 | C | CYS | B | 280 | 41.620 | 38.156 | 91.292 | 1.00 | 8.55 | 6 |
| ATOM | 625 | O | CYS | B | 280 | 40.735 | 37.826 | 92.094 | 1.00 | 10.52 | 8 |
| ATOM | 626 | CB | CYS | B | 280 | 43.482 | 37.053 | 92.640 | 1.00 | 11.29 | 6 |
| ATOM | 627 | SG | CYS | B | 280 | 45.205 | 37.119 | 93.235 | 1.00 | 15.57 | 16 |
| ATOM | 628 | N | VAL | B | 281 | 41.360 | 38.454 | 90.003 | 1.00 | 7.99 | 7 |
| ATOM | 629 | CA | VAL | B | 281 | 40.029 | 38.539 | 89.401 | 1.00 | 8.37 | 6 |
| ATOM | 630 | C | VAL | B | 281 | 39.594 | 37.286 | 88.649 | 1.00 | 9.01 | 6 |
| ATOM | 631 | O | VAL | B | 281 | 40.392 | 36.590 | 88.021 | 1.00 | 10.18 | 8 |
| ATOM | 632 | CB | VAL | B | 281 | 39.978 | 39.715 | 88.388 | 1.00 | 6.85 | 6 |
| ATOM | 633 | CG1 | VAL | B | 281 | 38.566 | 39.928 | 87.891 | 1.00 | 7.84 | 6 |
| ATOM | 634 | CG2 | VAL | B | 281 | 40.516 | 40.981 | 89.021 | 1.00 | 6.40 | 6 |
| ATOM | 635 | N | PHE | B | 282 | 38.301 | 37.029 | 88.743 | 1.00 | 10.93 | 7 |
| ATOM | 636 | CA | PHE | B | 282 | 37.672 | 35.924 | 88.028 | 1.00 | 11.84 | 6 |
| ATOM | 637 | C | PHE | B | 282 | 36.242 | 36.328 | 87.722 | 1.00 | 8.39 | 6 |
| ATOM | 638 | O | PHE | B | 282 | 35.701 | 37.253 | 88.329 | 1.00 | 8.10 | 8 |
| ATOM | 639 | CB | PHE | B | 282 | 37.698 | 34.617 | 88.843 | 1.00 | 13.30 | 6 |
| ATOM | 640 | CG | PHE | B | 282 | 36.692 | 34.527 | 89.967 | 1.00 | 15.63 | 6 |
| ATOM | 641 | CD1 | PHE | B | 282 | 37.023 | 34.981 | 91.243 | 1.00 | 15.64 | 6 |
| ATOM | 642 | CD2 | PHE | B | 282 | 35.420 | 33.998 | 89.753 | 1.00 | 12.04 | 6 |
| ATOM | 643 | CE1 | PHE | B | 282 | 36.101 | 34.910 | 92.288 | 1.00 | 15.69 | 6 |
| ATOM | 644 | CE2 | PHE | B | 282 | 34.493 | 33.925 | 90.789 | 1.00 | 14.14 | 6 |
| ATOM | 645 | CZ | PHE | B | 282 | 34.835 | 34.382 | 92.060 | 1.00 | 14.92 | 6 |
| ATOM | 646 | N | ALA | B | 283 | 35.663 | 35.637 | 86.790 | 1.00 | 6.99 | 7 |
| ATOM | 647 | CA | ALA | B | 283 | 34.291 | 35.923 | 86.409 | 1.00 | 8.48 | 6 |
| ATOM | 648 | C | ALA | B | 283 | 33.319 | 34.949 | 87.054 | 1.00 | 8.42 | 6 |
| ATOM | 649 | O | ALA | B | 283 | 33.575 | 33.743 | 87.089 | 1.00 | 5.63 | 8 |
| ATOM | 650 | CB | ALA | B | 283 | 34.157 | 35.879 | 84.892 | 1.00 | 3.99 | 6 |
| ATOM | 651 | N | ILE | B | 284 | 32.185 | 35.464 | 87.565 | 1.00 | 11.08 | 7 |
| ATOM | 652 | CA | ILE | B | 284 | 31.169 | 34.645 | 88.227 | 1.00 | 11.64 | 6 |
| ATOM | 653 | C | ILE | B | 284 | 30.447 | 33.819 | 87.164 | 1.00 | 13.24 | 6 |
| ATOM | 654 | O | ILE | B | 284 | 29.654 | 32.925 | 87.461 | 1.00 | 13.51 | 8 |
| ATOM | 655 | CB | ILE | B | 284 | 30.206 | 35.517 | 89.098 | 1.00 | 13.10 | 6 |
| ATOM | 656 | CG1 | ILE | B | 284 | 29.599 | 36.660 | 88.265 | 1.00 | 13.98 | 6 |
| ATOM | 657 | CG2 | ILE | B | 284 | 30.938 | 36.051 | 90.325 | 1.00 | 9.82 | 6 |
| ATOM | 658 | CD1 | ILE | B | 284 | 28.344 | 36.266 | 87.520 | 1.00 | 13.48 | 6 |
| ATOM | 659 | N | ASN | B | 285 | 30.746 | 34.126 | 85.925 | 1.00 | 14.49 | 7 |
| ATOM | 660 | CA | ASN | B | 285 | 30.125 | 33.369 | 84.848 | 1.00 | 17.12 | 6 |
| ATOM | 661 | C | ASN | B | 285 | 31.130 | 32.434 | 84.141 | 1.00 | 17.14 | 6 |
| ATOM | 662 | O | ASN | B | 285 | 30.736 | 31.596 | 83.331 | 1.00 | 20.42 | 8 |
| ATOM | 663 | CB | ASN | B | 285 | 29.474 | 34.315 | 83.841 | 1.00 | 16.59 | 6 |
| ATOM | 664 | CG | ASN | B | 285 | 30.486 | 35.056 | 82.990 | 1.00 | 17.52 | 6 |
| ATOM | 665 | OD1 | ASN | B | 285 | 31.636 | 35.215 | 83.377 | 1.00 | 16.16 | 8 |
| ATOM | 666 | ND2 | ASN | B | 285 | 30.055 | 35.508 | 81.810 | 1.00 | 19.89 | 7 |
| ATOM | 667 | N | ASN | B | 286 | 32.433 | 32.548 | 84.440 | 1.00 | 16.60 | 7 |
| ATOM | 668 | CA | ASN | B | 286 | 33.480 | 31.722 | 83.846 | 1.00 | 15.02 | 6 |
| ATOM | 669 | C | ASN | B | 286 | 34.139 | 30.890 | 84.913 | 1.00 | 16.06 | 6 |
| ATOM | 670 | O | ASN | B | 286 | 35.030 | 31.376 | 85.611 | 1.00 | 15.12 | 8 |
| ATOM | 671 | CB | ASN | B | 286 | 34.515 | 32.628 | 83.168 | 1.00 | 16.22 | 6 |
| ATOM | 672 | CG | ASN | B | 286 | 35.717 | 31.920 | 82.569 | 1.00 | 18.01 | 6 |
| ATOM | 673 | OD1 | ASN | B | 286 | 35.795 | 30.690 | 82.579 | 1.00 | 19.15 | 8 |
| ATOM | 674 | ND2 | ASN | B | 286 | 36.644 | 32.705 | 82.027 | 1.00 | 19.29 | 7 |
| ATOM | 675 | N | THR | B | 287 | 33.710 | 29.649 | 85.078 | 1.00 | 15.22 | 7 |
| ATOM | 676 | CA | THR | B | 287 | 34.234 | 28.799 | 86.142 | 1.00 | 15.55 | 6 |
| ATOM | 677 | C | THR | B | 287 | 35.700 | 28.605 | 86.001 | 1.00 | 13.49 | 6 |
| ATOM | 678 | O | THR | B | 287 | 36.474 | 28.921 | 86.905 | 1.00 | 13.57 | 8 |
| ATOM | 679 | CB | THR | B | 287 | 33.592 | 27.457 | 86.097 | 1.00 | 16.78 | 6 |
| ATOM | 680 | OG1 | THR | B | 287 | 32.194 | 27.607 | 86.323 | 1.00 | 19.11 | 8 |
| ATOM | 681 | CG2 | THR | B | 287 | 34.198 | 26.555 | 87.149 | 1.00 | 20.00 | 6 |
| ATOM | 682 | N | LYS | B | 288 | 36.082 | 28.100 | 84.864 | 1.00 | 13.23 | 7 |
| ATOM | 683 | CA | LYS | B | 288 | 37.462 | 27.911 | 84.630 | 1.00 | 16.48 | 6 |
| ATOM | 684 | C | LYS | B | 288 | 38.287 | 29.007 | 85.279 | 1.00 | 15.41 | 6 |
| ATOM | 685 | O | LYS | B | 288 | 38.984 | 28.780 | 86.270 | 1.00 | 17.54 | 8 |
| ATOM | 686 | CB | LYS | B | 288 | 37.703 | 27.873 | 83.127 | 1.00 | 18.67 | 6 |
| ATOM | 687 | CG | LYS | B | 288 | 38.674 | 26.789 | 82.712 | 1.00 | 27.96 | 6 |
| ATOM | 688 | CD | LYS | B | 288 | 40.099 | 27.190 | 83.091 | 1.00 | 34.28 | 6 |
| ATOM | 689 | CE | LYS | B | 288 | 41.111 | 26.105 | 82.744 | 1.00 | 37.35 | 6 |
| ATOM | 690 | NZ | LYS | B | 288 | 41.045 | 24.955 | 83.691 | 1.00 | 38.62 | 7 |
| ATOM | 691 | N | SER | B | 289 | 38.186 | 30.203 | 84.685 | 1.00 | 14.60 | 7 |
| ATOM | 692 | CA | SER | B | 289 | 38.923 | 31.358 | 85.151 | 1.00 | 13.62 | 6 |
| ATOM | 693 | C | SER | B | 289 | 39.014 | 31.316 | 86.644 | 1.00 | 13.51 | 6 |
| ATOM | 694 | O | SER | B | 289 | 39.880 | 31.972 | 87.218 | 1.00 | 11.81 | 8 |
| ATOM | 695 | CB | SER | B | 289 | 38.283 | 32.658 | 84.679 | 1.00 | 7.70 | 6 |
| ATOM | 696 | OG | SER | B | 289 | 37.051 | 32.900 | 85.339 | 1.00 | 14.33 | 8 |
| ATOM | 697 | N | PHE | B | 290 | 38.162 | 30.566 | 87.272 | 1.00 | 16.19 | 7 |
| ATOM | 698 | CA | PHE | B | 290 | 38.177 | 30.511 | 88.710 | 1.00 | 20.54 | 6 |
| ATOM | 699 | C | PHE | B | 290 | 39.284 | 29.589 | 89.204 | 1.00 | 23.94 | 6 |
| ATOM | 700 | O | PHE | B | 290 | 40.243 | 29.997 | 89.871 | 1.00 | 26.17 | 8 |
| ATOM | 701 | CB | PHE | B | 290 | 36.823 | 30.097 | 89.202 | 1.00 | 21.91 | 6 |
| ATOM | 702 | CG | PHE | B | 290 | 36.894 | 29.870 | 90.660 | 1.00 | 23.19 | 6 |
| ATOM | 703 | CD1 | PHE | B | 290 | 37.068 | 30.937 | 91.537 | 1.00 | 24.20 | 6 |
| ATOM | 704 | CD2 | PHE | B | 290 | 36.778 | 28.585 | 91.156 | 1.00 | 24.48 | 6 |
| ATOM | 705 | CE1 | PHE | B | 290 | 37.126 | 30.728 | 92.883 | 1.00 | 24.07 | 6 |
| ATOM | 706 | CE2 | PHE | B | 290 | 36.836 | 28.366 | 92.530 | 1.00 | 23.65 | 6 |
| ATOM | 707 | CZ | PHE | B | 290 | 37.010 | 29.442 | 93.395 | 1.00 | 24.70 | 6 |
| ATOM | 708 | N | GLU | B | 291 | 39.103 | 28.325 | 88.837 | 1.00 | 25.09 | 7 |
| ATOM | 709 | CA | GLU | B | 291 | 40.049 | 27.295 | 89.187 | 1.00 | 25.94 | 6 |
| ATOM | 710 | C | GLU | B | 291 | 41.403 | 27.887 | 88.930 | 1.00 | 26.14 | 6 |
| ATOM | 711 | O | GLU | B | 291 | 42.392 | 27.567 | 89.603 | 1.00 | 28.61 | 8 |
| ATOM | 712 | CB | GLU | B | 291 | 39.885 | 26.018 | 88.346 | 1.00 | 26.99 | 6 |
| ATOM | 713 | CG | GLU | B | 291 | 38.516 | 25.391 | 88.483 | 1.00 | 32.52 | 6 |
| ATOM | 714 | CD | GLU | B | 291 | 38.256 | 24.273 | 87.488 | 1.00 | 38.36 | 6 |
| ATOM | 715 | OE1 | GLU | B | 291 | 38.969 | 24.180 | 86.472 | 1.00 | 39.10 | 8 |
| ATOM | 716 | OE2 | GLU | B | 291 | 37.323 | 23.488 | 87.727 | 1.00 | 39.23 | 8 |
| ATOM | 717 | N | ASP | B | 292 | 41.410 | 28.760 | 87.944 | 1.00 | 24.07 | 7 |
| ATOM | 718 | CA | ASP | B | 292 | 42.632 | 29.400 | 87.560 | 1.00 | 22.65 | 6 |
| ATOM | 719 | C | ASP | B | 292 | 43.135 | 30.300 | 88.692 | 1.00 | 21.52 | 6 |
| ATOM | 720 | O | ASP | B | 292 | 44.342 | 30.320 | 88.962 | 1.00 | 18.98 | 8 |
| ATOM | 721 | CB | ASP | B | 292 | 42.426 | 30.211 | 86.294 | 1.00 | 22.60 | 6 |
| ATOM | 722 | CG | ASP | B | 292 | 42.621 | 29.373 | 85.047 | 1.00 | 24.88 | 6 |
| ATOM | 723 | OD1 | ASP | B | 292 | 43.200 | 28.275 | 85.154 | 1.00 | 26.11 | 8 |
| ATOM | 724 | OD2 | ASP | B | 292 | 42.186 | 29.811 | 83.948 | 1.00 | 27.57 | 8 |
| ATOM | 725 | N | ILE | B | 293 | 42.229 | 31.033 | 89.335 | 1.00 | 21.66 | 7 |
| ATOM | 726 | CA | ILE | B | 293 | 42.642 | 31.926 | 90.389 | 1.00 | 22.90 | 6 |
| ATOM | 727 | C | ILE | B | 293 | 43.410 | 31.156 | 91.471 | 1.00 | 20.93 | 6 |
| ATOM | 728 | O | ILE | B | 293 | 43.951 | 31.773 | 92.377 | 1.00 | 20.20 | 8 |
| ATOM | 729 | CB | ILE | B | 293 | 41.408 | 32.672 | 90.961 | 1.00 | 23.92 | 6 |
| ATOM | 730 | CG1 | ILE | B | 293 | 41.774 | 34.116 | 91.301 | 1.00 | 27.22 | 6 |
| ATOM | 731 | CG2 | ILE | B | 293 | 40.861 | 31.969 | 92.197 | 1.00 | 26.92 | 6 |
| ATOM | 732 | CD1 | ILE | B | 293 | 42.411 | 34.865 | 90.159 | 1.00 | 26.07 | 6 |
| ATOM | 733 | N | HIS | B | 294 | 43.471 | 29.817 | 91.406 | 1.00 | 20.43 | 7 |
| ATOM | 734 | CA | HIS | B | 294 | 44.207 | 29.015 | 92.411 | 1.00 | 21.44 | 6 |
| ATOM | 735 | C | HIS | B | 294 | 45.662 | 29.135 | 92.133 | 1.00 | 20.03 | 6 |
| ATOM | 736 | O | HIS | B | 294 | 46.498 | 29.405 | 92.983 | 1.00 | 18.65 | 8 |
| ATOM | 737 | CB | HIS | B | 294 | 43.919 | 27.525 | 92.255 | 1.00 | 25.77 | 6 |
| ATOM | 738 | CG | HIS | B | 294 | 42.434 | 27.217 | 92.436 | 1.00 | 29.47 | 6 |
| ATOM | 739 | ND1 | HIS | B | 294 | 41.428 | 28.071 | 92.036 | 1.00 | 30.04 | 7 |
| ATOM | 740 | CD2 | HIS | B | 294 | 41.816 | 26.160 | 93.019 | 1.00 | 31.40 | 6 |
| ATOM | 741 | CE1 | HIS | B | 294 | 40.256 | 27.552 | 92.359 | 1.00 | 31.12 | 6 |
| ATOM | 742 | NE2 | HIS | B | 294 | 40.463 | 26.394 | 92.957 | 1.00 | 31.99 | 7 |
| ATOM | 743 | N | GLN | B | 295 | 45.902 | 28.933 | 90.862 | 1.00 | 18.43 | 7 |
| ATOM | 744 | CA | GLN | B | 295 | 47.214 | 28.978 | 90.254 | 1.00 | 19.41 | 6 |
| ATOM | 745 | C | GLN | B | 295 | 47.914 | 30.340 | 90.463 | 1.00 | 17.28 | 6 |
| ATOM | 746 | O | GLN | B | 295 | 49.045 | 30.393 | 90.955 | 1.00 | 16.23 | 8 |
| ATOM | 747 | CB | GLN | B | 295 | 47.111 | 28.630 | 88.796 | 1.00 | 23.63 | 6 |
| ATOM | 748 | CG | GLN | B | 295 | 46.125 | 27.494 | 88.552 | 1.00 | 29.09 | 6 |
| ATOM | 749 | CD | GLN | B | 295 | 46.595 | 26.180 | 89.133 | 1.00 | 33.99 | 6 |
| ATOM | 750 | OE1 | GLN | B | 295 | 46.102 | 25.726 | 90.162 | 1.00 | 40.04 | 8 |
| ATOM | 751 | NE2 | GLN | B | 295 | 47.546 | 25.396 | 88.625 | 1.00 | 36.69 | 7 |
| ATOM | 752 | N | TYR | B | 296 | 47.257 | 31.423 | 90.115 | 1.00 | 14.91 | 7 |
| ATOM | 753 | CA | TYR | B | 296 | 47.837 | 32.713 | 90.299 | 1.00 | 14.09 | 6 |
| ATOM | 754 | C | TYR | B | 296 | 48.242 | 32.938 | 91.800 | 1.00 | 15.50 | 6 |
| ATOM | 755 | O | TYR | B | 296 | 49.353 | 33.410 | 92.086 | 1.00 | 16.74 | 8 |
| ATOM | 756 | CB | TYR | B | 296 | 46.878 | 33.752 | 89.640 | 1.00 | 11.04 | 6 |
| ATOM | 757 | CG | TYR | B | 296 | 46.899 | 33.720 | 88.101 | 1.00 | 13.49 | 6 |
| ATOM | 758 | CD1 | TYR | B | 296 | 47.907 | 34.404 | 87.419 | 1.00 | 13.01 | 6 |
| ATOM | 759 | CD2 | TYR | B | 296 | 45.948 | 33.031 | 87.352 | 1.00 | 14.29 | 6 |
| ATOM | 760 | CE1 | TYR | B | 296 | 47.972 | 34.398 | 86.036 | 1.00 | 13.01 | 6 |
| ATOM | 761 | CE2 | TYR | B | 296 | 46.006 | 33.016 | 85.958 | 1.00 | 14.55 | 6 |
| ATOM | 762 | CZ | TYR | B | 296 | 47.025 | 33.703 | 85.310 | 1.00 | 16.45 | 6 |
| ATOM | 763 | OH | TYR | B | 296 | 47.079 | 33.690 | 83.938 | 1.00 | 21.81 | 8 |
| ATOM | 764 | N | ARG | B | 297 | 47.349 | 32.609 | 92.759 | 1.00 | 16.79 | 7 |
| ATOM | 765 | CA | ARG | B | 297 | 47.642 | 32.792 | 94.215 | 1.00 | 19.11 | 6 |
| ATOM | 766 | C | ARG | B | 297 | 48.909 | 32.041 | 94.611 | 1.00 | 19.28 | 6 |
| ATOM | 767 | O | ARG | B | 297 | 49.685 | 32.523 | 95.446 | 1.00 | 16.61 | 8 |
| ATOM | 768 | CB | ARG | B | 297 | 46.457 | 32.315 | 95.110 | 1.00 | 24.36 | 6 |
| ATOM | 769 | CG | ARG | B | 297 | 46.243 | 33.059 | 96.427 | 1.00 | 28.41 | 6 |
| ATOM | 770 | CD | ARG | B | 297 | 47.235 | 32.656 | 97.517 | 1.00 | 34.72 | 6 |
| ATOM | 771 | NE | ARG | B | 297 | 46.599 | 32.095 | 98.726 | 1.00 | 40.89 | 7 |
| ATOM | 772 | CZ | ARG | B | 297 | 47.022 | 32.275 | 99.977 | 1.00 | 42.53 | 6 |
| ATOM | 773 | NH1 | ARG | B | 297 | 48.101 | 33.013 | 100.219 | 1.00 | 42.99 | 7 |
| ATOM | 774 | NH2 | ARG | B | 297 | 46.365 | 31.716 | 100.992 | 1.00 | 42.30 | 7 |
| ATOM | 775 | N | GLU | B | 298 | 49.107 | 30.856 | 94.022 | 1.00 | 18.62 | 7 |
| ATOM | 776 | CA | GLU | B | 298 | 50.266 | 30.062 | 94.385 | 1.00 | 20.41 | 6 |
| ATOM | 777 | C | GLU | B | 298 | 51.598 | 30.467 | 93.680 | 1.00 | 19.65 | 6 |
| ATOM | 778 | O | GLU | B | 298 | 52.657 | 30.256 | 94.275 | 1.00 | 20.78 | 8 |
| ATOM | 779 | CB | GLU | B | 298 | 49.962 | 28.577 | 94.191 | 1.00 | 23.16 | 6 |
| ATOM | 780 | CG | GLU | B | 298 | 49.546 | 28.127 | 92.795 | 1.00 | 30.82 | 6 |
| ATOM | 781 | CD | GLU | B | 298 | 49.230 | 26.641 | 92.685 | 1.00 | 36.03 | 6 |
| ATOM | 782 | OE1 | GLU | B | 298 | 48.642 | 26.091 | 93.647 | 1.00 | 42.37 | 8 |
| ATOM | 783 | OE2 | GLU | B | 298 | 49.575 | 26.018 | 91.649 | 1.00 | 35.30 | 8 |
| ATOM | 784 | N | GLN | B | 299 | 51.592 | 31.044 | 92.452 | 1.00 | 18.00 | 7 |
| ATOM | 785 | CA | GLN | B | 299 | 52.852 | 31.493 | 91.796 | 1.00 | 19.09 | 6 |
| ATOM | 786 | C | GLN | B | 299 | 53.218 | 32.885 | 92.247 | 1.00 | 19.41 | 6 |
| ATOM | 787 | O | GLN | B | 299 | 54.136 | 33.519 | 91.723 | 1.00 | 19.62 | 8 |
| ATOM | 788 | CB | GLN | B | 299 | 52.801 | 31.523 | 90.253 | 1.00 | 21.04 | 6 |
| ATOM | 789 | CG | GLN | B | 299 | 54.101 | 32.054 | 89.597 | 1.00 | 23.94 | 6 |
| ATOM | 790 | CD | GLN | B | 299 | 54.005 | 32.413 | 88.105 | 1.00 | 28.64 | 6 |
| ATOM | 791 | OE1 | GLN | B | 299 | 54.973 | 32.854 | 87.490 | 1.00 | 30.41 | 8 |
| ATOM | 792 | NE2 | GLN | B | 299 | 52.930 | 32.282 | 87.347 | 1.00 | 26.45 | 7 |
| ATOM | 793 | N | ILE | B | 300 | 52.469 | 33.339 | 93.214 | 1.00 | 18.61 | 7 |
| ATOM | 794 | CA | ILE | B | 300 | 52.773 | 34.624 | 93.761 | 1.00 | 18.21 | 6 |
| ATOM | 795 | C | ILE | B | 300 | 53.516 | 34.375 | 95.018 | 1.00 | 18.07 | 6 |
| ATOM | 796 | O | ILE | B | 300 | 54.332 | 35.178 | 95.456 | 1.00 | 17.22 | 8 |
| ATOM | 797 | CB | ILE | B | 300 | 51.512 | 35.424 | 94.066 | 1.00 | 15.78 | 6 |
| ATOM | 798 | CG1 | ILE | B | 300 | 50.878 | 35.876 | 92.757 | 1.00 | 15.72 | 6 |
| ATOM | 799 | CG2 | ILE | B | 300 | 51.829 | 36.621 | 94.943 | 1.00 | 14.51 | 6 |
| ATOM | 800 | CD1 | ILE | B | 300 | 49.631 | 36.707 | 92.939 | 1.00 | 12.62 | 6 |
| ATOM | 801 | N | LYS | B | 301 | 53.239 | 33.258 | 95.604 | 1.00 | 20.69 | 7 |
| ATOM | 802 | CA | LYS | B | 301 | 53.943 | 32.952 | 96.812 | 1.00 | 23.14 | 6 |
| ATOM | 803 | C | LYS | B | 301 | 55.394 | 32.744 | 96.457 | 1.00 | 23.52 | 6 |
| ATOM | 804 | O | LYS | B | 301 | 56.278 | 33.488 | 96.899 | 1.00 | 24.82 | 8 |
| ATOM | 805 | CB | LYS | B | 301 | 53.353 | 31.741 | 97.509 | 1.00 | 25.27 | 6 |
| ATOM | 806 | CG | LYS | B | 301 | 51.854 | 31.833 | 97.582 | 1.00 | 29.79 | 6 |
| ATOM | 807 | CD | LYS | B | 301 | 51.260 | 30.734 | 98.440 | 1.00 | 32.39 | 6 |
| ATOM | 808 | CE | LYS | B | 301 | 51.661 | 29.353 | 97.936 | 1.00 | 33.03 | 6 |
| ATOM | 809 | NZ | LYS | B | 301 | 51.328 | 28.281 | 98.914 | 1.00 | 33.49 | 7 |
| ATOM | 810 | N | ARG | B | 302 | 55.601 | 31.714 | 95.657 | 1.00 | 24.99 | 7 |
| ATOM | 811 | CA | ARG | B | 302 | 56.895 | 31.287 | 95.118 | 1.00 | 29.10 | 6 |
| ATOM | 812 | C | ARG | B | 302 | 57.887 | 32.412 | 95.082 | 1.00 | 28.73 | 6 |
| ATOM | 813 | O | ARG | B | 302 | 58.783 | 32.526 | 95.912 | 1.00 | 31.22 | 8 |
| ATOM | 814 | CB | ARG | B | 302 | 56.690 | 30.708 | 93.713 | 1.00 | 32.23 | 6 |
| ATOM | 815 | CG | ARG | B | 302 | 57.953 | 30.188 | 93.043 | 1.00 | 39.66 | 6 |
| ATOM | 816 | CD | ARG | B | 302 | 57.656 | 29.640 | 91.647 | 1.00 | 46.37 | 6 |
| ATOM | 817 | NE | ARG | B | 302 | 56.709 | 28.527 | 91.698 | 1.00 | 52.02 | 7 |
| ATOM | 818 | CZ | ARG | B | 302 | 55.781 | 28.283 | 90.772 | 1.00 | 54.94 | 6 |
| ATOM | 819 | NH1 | ARG | B | 302 | 55.661 | 29.075 | 89.712 | 1.00 | 54.32 | 7 |
| ATOM | 820 | NH2 | ARG | B | 302 | 54.979 | 27.233 | 90.899 | 1.00 | 55.66 | 7 |
| ATOM | 821 | N | VAL | B | 303 | 57.695 | 33.212 | 94.056 | 1.00 | 27.82 | 7 |
| ATOM | 822 | CA | VAL | B | 303 | 58.504 | 34.390 | 93.779 | 1.00 | 26.23 | 6 |
| ATOM | 823 | C | VAL | B | 303 | 58.691 | 35.210 | 95.034 | 1.00 | 25.98 | 6 |
| ATOM | 824 | O | VAL | B | 303 | 59.755 | 35.241 | 95.642 | 1.00 | 28.38 | 8 |
| ATOM | 825 | CB | VAL | B | 303 | 57.882 | 35.214 | 92.677 | 1.00 | 23.16 | 6 |
| ATOM | 826 | CG1 | VAL | B | 303 | 58.763 | 36.384 | 92.307 | 1.00 | 23.59 | 6 |
| ATOM | 827 | CG2 | VAL | B | 303 | 57.628 | 34.338 | 91.455 | 1.00 | 24.44 | 6 |
| ATOM | 828 | N | LYS | B | 304 | 57.623 | 35.867 | 95.408 | 1.00 | 25.53 | 7 |
| ATOM | 829 | CA | LYS | B | 304 | 57.670 | 36.645 | 96.577 | 1.00 | 25.49 | 6 |
| ATOM | 830 | C | LYS | B | 304 | 58.117 | 35.791 | 97.760 | 1.00 | 26.69 | 6 |
| ATOM | 831 | O | LYS | B | 304 | 58.681 | 36.316 | 98.724 | 1.00 | 29.45 | 8 |
| ATOM | 832 | CB | LYS | B | 304 | 56.322 | 37.296 | 96.834 | 1.00 | 23.46 | 6 |
| ATOM | 833 | CG | LYS | B | 304 | 55.772 | 38.098 | 95.654 | 1.00 | 22.09 | 6 |
| ATOM | 834 | CD | LYS | B | 304 | 54.750 | 39.111 | 96.115 | 1.00 | 22.17 | 6 |
| ATOM | 835 | CE | LYS | B | 304 | 55.412 | 40.358 | 96.674 | 1.00 | 18.46 | 6 |
| ATOM | 836 | NZ | LYS | B | 304 | 56.105 | 41.146 | 95.615 | 1.00 | 15.26 | 7 |
| ATOM | 837 | N | ASP | B | 305 | 57.893 | 34.477 | 97.719 | 1.00 | 25.00 | 7 |
| ATOM | 838 | CA | ASP | B | 305 | 58.265 | 33.681 | 98.861 | 1.00 | 24.80 | 6 |
| ATOM | 839 | C | ASP | B | 305 | 57.669 | 34.284 | 100.110 | 1.00 | 24.89 | 6 |
| ATOM | 840 | O | ASP | B | 305 | 58.357 | 34.845 | 100.979 | 1.00 | 20.71 | 8 |
| ATOM | 841 | CB | ASP | B | 305 | 59.790 | 33.568 | 98.973 | 1.00 | 28.25 | 6 |
| ATOM | 842 | CG | ASP | B | 305 | 60.239 | 32.534 | 99.982 | 1.00 | 33.23 | 6 |
| ATOM | 843 | OD1 | ASP | B | 305 | 59.562 | 31.496 | 100.102 | 1.00 | 33.95 | 8 |
| ATOM | 844 | OD2 | ASP | B | 305 | 61.285 | 32.754 | 100.652 | 1.00 | 34.62 | 8 |
| ATOM | 845 | N | SER | B | 306 | 56.341 | 34.151 | 100.165 | 1.00 | 25.98 | 7 |
| ATOM | 846 | CA | SER | B | 306 | 55.493 | 34.689 | 101.223 | 1.00 | 30.29 | 6 |
| ATOM | 847 | C | SER | B | 306 | 54.074 | 34.093 | 101.324 | 1.00 | 33.52 | 6 |
| ATOM | 848 | O | SER | B | 306 | 53.396 | 33.922 | 100.308 | 1.00 | 33.04 | 8 |
| ATOM | 849 | CB | SER | B | 306 | 55.393 | 36.213 | 101.046 | 1.00 | 30.37 | 6 |
| ATOM | 850 | OG | SER | B | 306 | 54.882 | 36.833 | 102.214 | 1.00 | 31.79 | 8 |
| ATOM | 851 | N | ASP | B | 307 | 53.659 | 33.761 | 102.569 | 1.00 | 36.46 | 7 |
| ATOM | 852 | CA | ASP | B | 307 | 52.329 | 33.236 | 102.930 | 1.00 | 38.66 | 6 |
| ATOM | 853 | C | ASP | B | 307 | 51.451 | 34.459 | 103.301 | 1.00 | 37.84 | 6 |
| ATOM | 854 | O | ASP | B | 307 | 50.268 | 34.334 | 103.583 | 1.00 | 38.03 | 8 |
| ATOM | 855 | CB | ASP | B | 307 | 52.401 | 32.228 | 104.103 | 1.00 | 43.58 | 6 |
| ATOM | 856 | CG | ASP | B | 307 | 53.136 | 30.936 | 103.745 | 1.00 | 48.79 | 6 |
| ATOM | 857 | OD1 | ASP | B | 307 | 52.598 | 30.161 | 102.924 | 1.00 | 50.11 | 8 |
| ATOM | 858 | OD2 | ASP | B | 307 | 54.226 | 30.696 | 104.292 | 1.00 | 51.33 | 8 |
| ATOM | 859 | N | ASP | B | 308 | 52.083 | 35.644 | 103.305 | 1.00 | 36.46 | 7 |
| ATOM | 860 | CA | ASP | B | 308 | 51.395 | 36.928 | 103.576 | 1.00 | 36.34 | 6 |
| ATOM | 861 | C | ASP | B | 308 | 51.653 | 37.997 | 102.534 | 1.00 | 30.99 | 6 |
| ATOM | 862 | O | ASP | B | 308 | 52.464 | 38.897 | 102.711 | 1.00 | 29.55 | 8 |
| ATOM | 863 | CB | ASP | B | 308 | 51.730 | 37.467 | 104.972 | 1.00 | 40.75 | 6 |
| ATOM | 864 | CG | ASP | B | 308 | 50.575 | 38.336 | 105.494 | 1.00 | 45.02 | 6 |
| ATOM | 865 | OD1 | ASP | B | 308 | 50.552 | 39.542 | 105.170 | 1.00 | 48.17 | 8 |
| ATOM | 866 | OD2 | ASP | B | 308 | 49.717 | 37.816 | 106.221 | 1.00 | 45.72 | 8 |
| ATOM | 867 | N | VAL | B | 309 | 50.921 | 37.874 | 101.447 | 1.00 | 27.11 | 7 |
| ATOM | 868 | CA | VAL | B | 309 | 51.040 | 38.828 | 100.376 | 1.00 | 21.48 | 6 |
| ATOM | 869 | C | VAL | B | 309 | 49.741 | 39.577 | 100.251 | 1.00 | 18.32 | 6 |
| ATOM | 870 | O | VAL | B | 309 | 48.645 | 39.034 | 100.447 | 1.00 | 17.92 | 8 |
| ATOM | 871 | CB | VAL | B | 309 | 51.321 | 38.118 | 99.025 | 1.00 | 21.27 | 6 |
| ATOM | 872 | CG1 | VAL | B | 309 | 51.520 | 39.145 | 97.914 | 1.00 | 18.26 | 6 |
| ATOM | 873 | CG2 | VAL | B | 309 | 52.529 | 37.195 | 99.138 | 1.00 | 19.59 | 6 |
| ATOM | 874 | N | PRO | B | 310 | 49.879 | 40.860 | 99.921 | 1.00 | 16.40 | 7 |
| ATOM | 875 | CA | PRO | B | 310 | 48.670 | 41.712 | 99.670 | 1.00 | 16.13 | 6 |
| ATOM | 876 | C | PRO | B | 310 | 47.745 | 41.187 | 98.570 | 1.00 | 15.20 | 6 |
| ATOM | 877 | O | PRO | B | 310 | 48.088 | 41.253 | 97.388 | 1.00 | 14.88 | 8 |
| ATOM | 878 | CB | PRO | B | 310 | 49.279 | 43.078 | 99.484 | 1.00 | 16.76 | 6 |
| ATOM | 879 | CG | PRO | B | 310 | 50.396 | 43.055 | 100.489 | 1.00 | 16.46 | 6 |
| ATOM | 880 | CD | PRO | B | 310 | 50.793 | 41.637 | 100.764 | 1.00 | 17.57 | 6 |
| ATOM | 881 | N | MET | B | 311 | 46.610 | 40.687 | 98.963 | 1.00 | 15.46 | 7 |
| ATOM | 882 | CA | MET | B | 311 | 45.743 | 40.158 | 97.980 | 1.00 | 17.63 | 6 |
| ATOM | 883 | C | MET | B | 311 | 44.322 | 40.402 | 98.272 | 1.00 | 16.15 | 6 |
| ATOM | 684 | O | MET | B | 311 | 43.947 | 40.926 | 99.323 | 1.00 | 17.35 | 8 |
| ATOM | 885 | CB | MET | B | 311 | 45.962 | 38.660 | 97.849 | 1.00 | 16.52 | 6 |
| ATOM | 886 | CG | MET | B | 311 | 46.959 | 38.273 | 96.790 | 1.00 | 21.56 | 6 |
| ATOM | 887 | SD | MET | B | 311 | 47.610 | 36.613 | 97.073 | 1.00 | 22.66 | 16 |
| ATOM | 888 | CE | MET | B | 311 | 48.602 | 36.392 | 95.605 | 1.00 | 23.62 | 6 |
| ATOM | 889 | N | VAL | B | 312 | 43.536 | 40.016 | 97.338 | 1.00 | 16.14 | 7 |
| ATOM | 890 | CA | VAL | B | 312 | 42.158 | 40.232 | 97.457 | 1.00 | 14.33 | 6 |
| ATOM | 891 | C | VAL | B | 312 | 41.548 | 39.408 | 96.357 | 1.00 | 15.23 | 6 |
| ATOM | 892 | O | VAL | B | 312 | 42.098 | 39.330 | 95.267 | 1.00 | 15.07 | 8 |
| ATOM | 893 | CB | VAL | B | 312 | 41.803 | 41.728 | 97.284 | 1.00 | 12.34 | 6 |
| ATOM | 894 | CG1 | VAL | B | 312 | 40.281 | 41.915 | 97.241 | 1.00 | 10.09 | 6 |
| ATOM | 895 | CG2 | VAL | B | 312 | 42.418 | 42.564 | 98.408 | 1.00 | 12.76 | 6 |
| ATOM | 896 | N | LEU | B | 313 | 40.413 | 38.765 | 96.632 | 1.00 | 14.69 | 7 |
| ATOM | 897 | CA | LEU | B | 313 | 39.708 | 37.920 | 95.621 | 1.00 | 14.64 | 6 |
| ATOM | 898 | C | LEU | B | 313 | 38.525 | 38.697 | 95.032 | 1.00 | 14.42 | 6 |
| ATOM | 899 | O | LEU | B | 313 | 37.662 | 39.146 | 95.783 | 1.00 | 14.55 | 8 |
| ATOM | 900 | CB | LEU | B | 313 | 39.245 | 36.615 | 96.275 | 1.00 | 13.14 | 6 |
| ATOM | 901 | CG | LEU | B | 313 | 38.647 | 35.566 | 95.352 | 1.00 | 14.44 | 6 |
| ATOM | 902 | CD1 | LEU | B | 313 | 39.699 | 34.972 | 94.432 | 1.00 | 14.50 | 6 |
| ATOM | 903 | CD2 | LEU | B | 313 | 37.976 | 34.475 | 96.164 | 1.00 | 15.81 | 6 |
| ATOM | 904 | N | VAL | B | 314 | 38.500 | 38.864 | 93.713 | 1.00 | 14.99 | 7 |
| ATOM | 905 | CA | VAL | B | 314 | 37.432 | 39.607 | 93.055 | 1.00 | 11.91 | 6 |
| ATOM | 906 | C | VAL | B | 314 | 36.583 | 38.766 | 92.110 | 1.00 | 10.69 | 6 |
| ATOM | 907 | O | VAL | B | 314 | 37.101 | 38.113 | 91.211 | 1.00 | 11.82 | 8 |
| ATOM | 908 | CB | VAL | B | 314 | 38.000 | 40.825 | 92.281 | 1.00 | 11.45 | 6 |
| ATOM | 909 | CG1 | VAL | B | 314 | 36.900 | 41.536 | 91.518 | 1.00 | 12.32 | 6 |
| ATOM | 910 | CG2 | VAL | B | 314 | 38.674 | 41.788 | 93.251 | 1.00 | 11.49 | 6 |
| ATOM | 911 | N | GLY | B | 315 | 35.273 | 38.788 | 92.346 | 1.00 | 11.35 | 7 |
| ATOM | 912 | CA | GLY | B | 315 | 34.330 | 38.077 | 91.504 | 1.00 | 9.22 | 6 |
| ATOM | 913 | C | GLY | B | 315 | 33.689 | 39.127 | 90.615 | 1.00 | 12.51 | 6 |
| ATOM | 914 | O | GLY | B | 315 | 32.731 | 39.799 | 91.017 | 1.00 | 12.53 | 8 |
| ATOM | 915 | N | ASN | B | 316 | 34.237 | 39.287 | 89.414 | 1.00 | 10.62 | 7 |
| ATOM | 916 | CA | ASN | B | 316 | 33.748 | 40.285 | 88.469 | 1.00 | 10.66 | 6 |
| ATOM | 917 | C | ASN | B | 316 | 32.563 | 39.799 | 87.642 | 1.00 | 11.60 | 6 |
| ATOM | 918 | O | ASN | B | 316 | 32.296 | 38.595 | 87.566 | 1.00 | 11.45 | 8 |
| ATOM | 919 | CB | ASN | B | 316 | 34.893 | 40.744 | 87.549 | 1.00 | 6.83 | 6 |
| ATOM | 920 | CG | ASN | B | 316 | 34.547 | 41.995 | 86.760 | 1.00 | 7.52 | 6 |
| ATOM | 921 | OD1 | ASN | B | 316 | 34.029 | 42.962 | 87.310 | 1.00 | 9.62 | 8 |
| ATOM | 922 | ND2 | ASN | B | 316 | 34.834 | 41.980 | 85.462 | 1.00 | 6.51 | 7 |
| ATOM | 923 | N | LYS | B | 317 | 31.888 | 40.755 | 87.003 | 1.00 | 11.29 | 7 |
| ATOM | 924 | CA | LYS | B | 317 | 30.721 | 40.505 | 86.159 | 1.00 | 11.62 | 6 |
| ATOM | 925 | C | LYS | B | 317 | 29.506 | 40.053 | 86.974 | 1.00 | 14.05 | 6 |
| ATOM | 926 | O | LYS | B | 317 | 28.733 | 39.201 | 86.530 | 1.00 | 14.80 | 8 |
| ATOM | 927 | CB | LYS | B | 317 | 31.049 | 39.478 | 85.065 | 1.00 | 11.25 | 6 |
| ATOM | 928 | CG | LYS | B | 317 | 32.190 | 39.886 | 84.146 | 1.00 | 15.13 | 6 |
| ATOM | 929 | CD | LYS | B | 317 | 32.355 | 38.911 | 83.000 | 1.00 | 15.16 | 6 |
| ATOM | 930 | CE | LYS | B | 317 | 33.537 | 39.291 | 82.121 | 1.00 | 15.28 | 6 |
| ATOM | 931 | NZ | LYS | B | 317 | 33.660 | 38.392 | 80.939 | 1.00 | 11.37 | 7 |
| ATOM | 932 | N | CYS | B | 318 | 29.325 | 40.633 | 88.163 | 1.00 | 14.30 | 7 |
| ATOM | 933 | CA | CYS | B | 318 | 28.211 | 40.281 | 89.032 | 1.00 | 15.00 | 6 |
| ATOM | 934 | C | CYS | B | 318 | 26.903 | 40.873 | 88.524 | 1.00 | 14.73 | 6 |
| ATOM | 935 | O | CYS | B | 318 | 25.837 | 40.685 | 89.137 | 1.00 | 15.53 | 8 |
| ATOM | 936 | CB | CYS | B | 318 | 28.482 | 40.747 | 90.463 | 1.00 | 14.78 | 6 |
| ATOM | 937 | SG | CYS | B | 318 | 29.868 | 39.913 | 91.271 | 1.00 | 21.51 | 16 |
| ATOM | 938 | N | ASP | B | 319 | 26.950 | 41.595 | 87.413 | 1.00 | 15.68 | 7 |
| ATOM | 939 | CA | ASP | B | 319 | 25.767 | 42.195 | 86.805 | 1.00 | 17.22 | 6 |
| ATOM | 940 | C | ASP | B | 319 | 25.043 | 41.167 | 85.929 | 1.00 | 19.24 | 6 |
| ATOM | 941 | O | ASP | B | 319 | 23.852 | 41.307 | 85.645 | 1.00 | 20.82 | 8 |
| ATOM | 942 | CB | ASP | B | 319 | 26.169 | 43.404 | 85.957 | 1.00 | 17.35 | 6 |
| ATOM | 943 | CG | ASP | B | 319 | 27.175 | 43.049 | 84.883 | 1.00 | 16.69 | 6 |
| ATOM | 944 | OD1 | ASP | B | 319 | 28.385 | 43.018 | 85.187 | 1.00 | 14.67 | 8 |
| ATOM | 945 | OD2 | ASP | B | 319 | 26.755 | 42.783 | 83.741 | 1.00 | 13.22 | 8 |
| ATOM | 946 | N | LEU | B | 320 | 25.777 | 40.146 | 85.495 | 1.00 | 20.76 | 7 |
| ATOM | 947 | CA | LEU | B | 320 | 25.232 | 39.086 | 84.652 | 1.00 | 20.59 | 6 |
| ATOM | 948 | C | LEU | B | 320 | 24.428 | 38.086 | 85.473 | 1.00 | 22.70 | 6 |
| ATOM | 949 | O | LEU | B | 320 | 24.870 | 37.639 | 86.531 | 1.00 | 22.34 | 8 |
| ATOM | 950 | CB | LEU | B | 320 | 26.359 | 38.367 | 83.910 | 1.00 | 20.19 | 6 |
| ATOM | 951 | CG | LEU | B | 320 | 27.196 | 39.216 | 82.947 | 1.00 | 22.49 | 6 |
| ATOM | 952 | CD1 | LEU | B | 320 | 28.331 | 38.387 | 82.364 | 1.00 | 22.21 | 6 |
| ATOM | 953 | CD2 | LEU | B | 320 | 26.320 | 39.773 | 81.834 | 1.00 | 20.51 | 6 |
| ATOM | 954 | N | ALA | B | 321 | 23.250 | 37.698 | 84.969 | 1.00 | 24.37 | 7 |
| ATOM | 955 | CA | ALA | B | 321 | 22.362 | 36.760 | 85.648 | 1.00 | 25.35 | 6 |
| ATOM | 956 | C | ALA | B | 321 | 22.850 | 35.303 | 85.543 | 1.00 | 25.06 | 6 |
| ATOM | 957 | O | ALA | B | 321 | 22.642 | 34.523 | 86.488 | 1.00 | 24.31 | 8 |
| ATOM | 958 | CB | ALA | B | 321 | 20.946 | 36.894 | 85.095 | 1.00 | 25.21 | 6 |
| ATOM | 959 | N | ALA | B | 322 | 23.499 | 34.891 | 84.442 | 1.00 | 27.03 | 7 |
| ATOM | 960 | CA | ALA | B | 322 | 24.041 | 33.524 | 84.351 | 1.00 | 27.13 | 6 |
| ATOM | 961 | C | ALA | B | 322 | 25.325 | 33.427 | 85.159 | 1.00 | 28.15 | 6 |
| ATOM | 962 | O | ALA | B | 322 | 26.342 | 34.022 | 84.804 | 1.00 | 28.32 | 8 |
| ATOM | 963 | CB | ALA | B | 322 | 24.303 | 33.130 | 82.909 | 1.00 | 28.01 | 6 |
| ATOM | 964 | N | ARG | B | 323 | 25.319 | 32.665 | 86.266 | 1.00 | 27.87 | 7 |
| ATOM | 965 | CA | ARG | B | 323 | 26.502 | 32.518 | 87.133 | 1.00 | 27.49 | 6 |
| ATOM | 966 | C | ARG | B | 323 | 26.871 | 31.058 | 87.236 | 1.00 | 28.01 | 6 |
| ATOM | 967 | O | ARG | B | 323 | 26.003 | 30.217 | 87.425 | 1.00 | 29.35 | 8 |
| ATOM | 968 | CB | ARG | B | 323 | 26.223 | 33.096 | 88.529 | 1.00 | 25.77 | 6 |
| ATOM | 969 | CG | ARG | B | 323 | 27.067 | 32.531 | 89.661 | 1.00 | 28.26 | 6 |
| ATOM | 970 | CD | ARG | B | 323 | 26.557 | 32.982 | 91.025 | 1.00 | 28.88 | 6 |
| ATOM | 971 | NE | ARG | B | 323 | 26.522 | 34.436 | 91.170 | 1.00 | 29.61 | 7 |
| ATOM | 972 | CZ | ARG | B | 323 | 27.514 | 35.138 | 91.712 | 1.00 | 29.96 | 6 |
| ATOM | 973 | NH1 | ARG | B | 323 | 28.601 | 34.527 | 92.161 | 1.00 | 30.42 | 7 |
| ATOM | 974 | NH2 | ARG | B | 323 | 27.411 | 36.455 | 91.792 | 1.00 | 28.26 | 7 |
| ATOM | 975 | N | THR | B | 324 | 28.125 | 30.789 | 87.106 | 1.00 | 27.10 | 7 |
| ATOM | 976 | CA | THR | B | 324 | 28.524 | 29.419 | 87.261 | 1.00 | 28.12 | 6 |
| ATOM | 977 | C | THR | B | 324 | 29.352 | 29.364 | 88.554 | 1.00 | 29.04 | 6 |
| ATOM | 978 | O | THR | B | 324 | 29.711 | 26.281 | 89.010 | 1.00 | 28.56 | 8 |
| ATOM | 979 | CB | THR | B | 324 | 29.285 | 28.956 | 86.017 | 1.00 | 26.48 | 6 |
| ATOM | 980 | OG1 | THR | B | 324 | 30.547 | 29.643 | 85.959 | 1.00 | 25.89 | 8 |
| ATOM | 981 | CG2 | THR | B | 324 | 28.479 | 29.248 | 84.755 | 1.00 | 26.61 | 6 |
| ATOM | 982 | N | VAL | B | 325 | 29.652 | 30.532 | 89.129 | 1.00 | 30.37 | 7 |
| ATOM | 983 | CA | VAL | B | 325 | 30.459 | 30.565 | 90.360 | 1.00 | 31.86 | 6 |
| ATOM | 984 | C | VAL | B | 325 | 29.808 | 31.333 | 91.489 | 1.00 | 34.18 | 6 |
| ATOM | 985 | O | VAL | B | 325 | 29.773 | 32.556 | 91.589 | 1.00 | 34.29 | 8 |
| ATOM | 986 | CB | VAL | B | 325 | 31.854 | 31.123 | 90.118 | 1.00 | 30.27 | 6 |
| ATOM | 987 | CG1 | VAL | B | 325 | 32.680 | 31.031 | 91.398 | 1.00 | 28.13 | 6 |
| ATOM | 988 | CG2 | VAL | B | 325 | 32.536 | 30.403 | 88.973 | 1.00 | 27.79 | 6 |
| ATOM | 989 | N | GLU | B | 326 | 29.296 | 30.457 | 92.313 | 1.00 | 36.05 | 7 |
| ATOM | 990 | CA | GLU | B | 326 | 28.547 | 30.657 | 93.527 | 1.00 | 37.94 | 6 |
| ATOM | 991 | C | GLU | B | 326 | 29.434 | 31.050 | 94.689 | 1.00 | 36.63 | 6 |
| ATOM | 992 | O | GLU | B | 326 | 30.157 | 30.274 | 95.307 | 1.00 | 34.91 | 8 |
| ATOM | 993 | CB | GLU | B | 326 | 27.859 | 29.359 | 93.873 | 1.00 | 40.60 | 6 |
| ATOM | 994 | CG | GLU | B | 326 | 28.863 | 28.220 | 93.698 | 1.00 | 43.96 | 6 |
| ATOM | 995 | CD | GLU | B | 326 | 28.222 | 26.856 | 93.537 | 1.00 | 46.02 | 6 |
| ATOM | 996 | OE1 | GLU | B | 326 | 27.162 | 26.618 | 94.170 | 1.00 | 44.64 | 8 |
| ATOM | 997 | OE2 | GLU | B | 326 | 28.767 | 26.011 | 92.800 | 1.00 | 47.53 | 8 |
| ATOM | 998 | N | SER | B | 327 | 29.301 | 32.315 | 94.927 | 1.00 | 35.64 | 7 |
| ATOM | 999 | CA | SER | B | 327 | 29.983 | 33.071 | 95.948 | 1.00 | 36.21 | 6 |
| ATOM | 1000 | C | SER | B | 327 | 30.604 | 32.249 | 97.116 | 1.00 | 36.71 | 6 |
| ATOM | 1001 | O | SER | B | 327 | 31.810 | 32.343 | 97.325 | 1.00 | 37.79 | 8 |
| ATOM | 1002 | CB | SER | B | 327 | 29.023 | 34.143 | 96.444 | 1.00 | 34.47 | 6 |
| ATOM | 1003 | OG | SER | B | 327 | 28.347 | 34.768 | 95.363 | 1.00 | 36.70 | 8 |
| ATOM | 1004 | N | ARG | B | 328 | 29.834 | 31.449 | 97.864 | 1.00 | 36.85 | 7 |
| ATOM | 1005 | CA | ARG | B | 328 | 30.323 | 30.666 | 99.009 | 1.00 | 37.15 | 6 |
| ATOM | 1006 | C | ARG | B | 328 | 31.701 | 29.991 | 98.770 | 1.00 | 35.23 | 6 |
| ATOM | 1007 | O | ARG | B | 328 | 32.637 | 30.199 | 99.540 | 1.00 | 33.28 | 8 |
| ATOM | 1008 | CB | ARG | B | 328 | 29.278 | 29.624 | 99.368 | 1.00 | 40.41 | 6 |
| ATOM | 1009 | CG | ARG | B | 328 | 29.594 | 28.789 | 100.601 | 1.00 | 47.12 | 6 |
| ATOM | 1010 | CD | ARG | B | 328 | 29.943 | 29.654 | 101.803 | 1.00 | 54.33 | 6 |
| ATOM | 1011 | NE | ARG | B | 328 | 28.927 | 30.666 | 102.091 | 1.00 | 58.74 | 7 |
| ATOM | 1012 | CZ | ARG | B | 328 | 29.126 | 31.666 | 102.948 | 1.00 | 60.74 | 6 |
| ATOM | 1013 | NH1 | ARG | B | 328 | 30.274 | 31.763 | 103.609 | 1.00 | 60.97 | 7 |
| ATOM | 1014 | NH2 | ARG | B | 328 | 28.185 | 32.584 | 103.128 | 1.00 | 61.45 | 7 |
| ATOM | 1015 | N | GLN | B | 329 | 31.796 | 29.191 | 97.701 | 1.00 | 33.86 | 7 |
| ATOM | 1016 | CA | GLN | B | 329 | 33.049 | 28.485 | 97.360 | 1.00 | 30.87 | 6 |
| ATOM | 1017 | C | GLN | B | 329 | 34.146 | 29.497 | 97.094 | 1.00 | 30.48 | 6 |
| ATOM | 1018 | O | GLN | B | 329 | 35.317 | 29.148 | 97.130 | 1.00 | 31.09 | 8 |
| ATOM | 1019 | CB | GLN | B | 329 | 32.820 | 27.483 | 96.203 | 1.00 | 31.54 | 6 |
| ATOM | 1020 | CG | GLN | B | 329 | 32.863 | 28.079 | 94.779 | 1.00 | 33.41 | 6 |
| ATOM | 1021 | CD | GLN | B | 329 | 32.556 | 27.067 | 93.675 | 1.00 | 35.16 | 6 |
| ATOM | 1022 | OE1 | GLN | B | 329 | 33.358 | 26.175 | 93.400 | 1.00 | 35.03 | 8 |
| ATOM | 1023 | NE2 | GLN | B | 329 | 31.450 | 27.012 | 92.939 | 1.00 | 32.98 | 7 |
| ATOM | 1024 | N | ALA | B | 330 | 33.777 | 30.728 | 96.841 | 1.00 | 29.35 | 7 |
| ATOM | 1025 | CA | ALA | B | 330 | 34.778 | 31.748 | 96.675 | 1.00 | 29.00 | 6 |
| ATOM | 1026 | C | ALA | B | 330 | 34.958 | 32.411 | 98.035 | 1.00 | 28.83 | 6 |
| ATOM | 1027 | O | ALA | B | 330 | 36.061 | 32.726 | 98.470 | 1.00 | 26.52 | 8 |
| ATOM | 1028 | CB | ALA | B | 330 | 34.360 | 32.778 | 95.638 | 1.00 | 25.88 | 6 |
| ATOM | 1029 | N | GLN | B | 331 | 33.839 | 32.590 | 98.688 | 1.00 | 29.03 | 7 |
| ATOM | 1030 | CA | GLN | B | 331 | 33.963 | 33.150 | 99.982 | 1.00 | 30.45 | 6 |
| ATOM | 1031 | C | GLN | B | 331 | 34.838 | 32.206 | 100.813 | 1.00 | 29.26 | 6 |
| ATOM | 1032 | O | GLN | B | 331 | 35.760 | 32.636 | 101.513 | 1.00 | 29.19 | 8 |
| ATOM | 1033 | CB | GLN | B | 331 | 32.583 | 33.373 | 100.609 | 1.00 | 32.66 | 6 |
| ATOM | 1034 | CG | GLN | B | 331 | 31.710 | 34.274 | 99.763 | 1.00 | 39.01 | 6 |
| ATOM | 1035 | CD | GLN | B | 331 | 30.500 | 34.733 | 100.496 | 1.00 | 42.03 | 6 |
| ATOM | 1036 | OE1 | GLN | B | 331 | 30.155 | 34.184 | 101.535 | 1.00 | 42.26 | 8 |
| ATOM | 1037 | NE2 | GLN | B | 331 | 29.683 | 35.716 | 100.136 | 1.00 | 42.84 | 7 |
| ATOM | 1038 | N | ASP | B | 332 | 34.578 | 30.899 | 100.741 | 1.00 | 28.82 | 7 |
| ATOM | 1039 | CA | ASP | B | 332 | 35.325 | 29.900 | 101.503 | 1.00 | 29.36 | 6 |
| ATOM | 1040 | C | ASP | B | 332 | 36.776 | 29.759 | 101.050 | 1.00 | 28.87 | 6 |
| ATOM | 1041 | O | ASP | B | 332 | 37.565 | 29.154 | 101.760 | 1.00 | 31.96 | 8 |
| ATOM | 1042 | CB | ASP | B | 332 | 34.641 | 28.525 | 101.401 | 1.00 | 30.32 | 6 |
| ATOM | 1043 | CG | ASP | B | 332 | 33.176 | 28.561 | 101.779 | 1.00 | 34.65 | 6 |
| ATOM | 1044 | OD1 | ASP | B | 332 | 32.752 | 29.525 | 102.445 | 1.00 | 38.52 | 8 |
| ATOM | 1045 | OD2 | ASP | B | 332 | 32.431 | 27.623 | 101.399 | 1.00 | 35.26 | 8 |
| ATOM | 1046 | N | LEU | B | 333 | 37.142 | 30.285 | 99.873 | 1.00 | 28.35 | 7 |
| ATOM | 1047 | CA | LEU | B | 333 | 38.538 | 30.120 | 99.432 | 1.00 | 26.00 | 6 |
| ATOM | 1048 | C | LEU | B | 333 | 39.447 | 31.151 | 100.026 | 1.00 | 24.97 | 6 |
| ATOM | 1049 | O | LEU | B | 333 | 40.598 | 30.892 | 100.384 | 1.00 | 24.07 | 8 |
| ATOM | 1050 | CB | LEU | B | 333 | 38.623 | 30.217 | 97.932 | 1.00 | 24.43 | 6 |
| ATOM | 1051 | CG | LEU | B | 333 | 40.043 | 30.371 | 97.417 | 1.00 | 23.13 | 6 |
| ATOM | 1052 | CD1 | LEU | B | 333 | 40.793 | 29.047 | 97.511 | 1.00 | 22.58 | 6 |
| ATOM | 1053 | CD2 | LEU | B | 333 | 40.026 | 30.877 | 95.979 | 1.00 | 21.94 | 6 |
| ATOM | 1054 | N | ALA | B | 334 | 38.906 | 32.361 | 100.143 | 1.00 | 23.89 | 7 |
| ATOM | 1055 | CA | ALA | B | 334 | 39.631 | 33.451 | 100.749 | 1.00 | 24.51 | 6 |
| ATOM | 1056 | C | ALA | B | 334 | 39.704 | 33.242 | 102.240 | 1.00 | 25.10 | 6 |
| ATOM | 1057 | O | ALA | B | 334 | 40.111 | 34.113 | 103.009 | 1.00 | 24.28 | 8 |
| ATOM | 1058 | CB | ALA | B | 334 | 38.979 | 34.800 | 100.432 | 1.00 | 25.48 | 6 |
| ATOM | 1059 | N | ARG | B | 335 | 39.283 | 32.069 | 102.639 | 1.00 | 25.04 | 7 |
| ATOM | 1060 | CA | ARG | B | 335 | 39.290 | 31.822 | 104.043 | 1.00 | 27.24 | 6 |
| ATOM | 1061 | C | ARG | B | 335 | 40.443 | 30.945 | 104.429 | 1.00 | 25.86 | 6 |
| ATOM | 1062 | O | ARG | B | 335 | 40.782 | 30.805 | 105.619 | 1.00 | 27.08 | 8 |
| ATOM | 1063 | CB | ARG | B | 335 | 37.954 | 31.252 | 104.459 | 1.00 | 30.11 | 6 |
| ATOM | 1064 | CG | ARG | B | 335 | 37.625 | 31.552 | 105.898 | 1.00 | 36.34 | 6 |
| ATOM | 1065 | CD | ARG | B | 335 | 37.316 | 30.244 | 106.550 | 1.00 | 41.48 | 6 |
| ATOM | 1066 | NE | ARG | B | 335 | 36.829 | 29.299 | 105.552 | 1.00 | 45.90 | 7 |
| ATOM | 1067 | CZ | ARG | B | 335 | 37.450 | 28.206 | 105.136 | 1.00 | 47.90 | 6 |
| ATOM | 1068 | NH1 | ARG | B | 335 | 38.622 | 27.895 | 105.663 | 1.00 | 48.82 | 7 |
| ATOM | 1069 | NH2 | ARG | B | 335 | 36.902 | 27.417 | 104.214 | 1.00 | 47.30 | 7 |
| ATOM | 1070 | N | SER | B | 336 | 41.031 | 30.358 | 103.416 | 1.00 | 23.50 | 7 |
| ATOM | 1071 | CA | SER | B | 336 | 42.210 | 29.580 | 103.570 | 1.00 | 22.24 | 6 |
| ATOM | 1072 | C | SER | B | 336 | 43.358 | 30.478 | 103.130 | 1.00 | 20.50 | 6 |
| ATOM | 1073 | O | SER | B | 336 | 44.438 | 30.454 | 103.721 | 1.00 | 20.92 | 8 |
| ATOM | 1074 | CB | SER | B | 336 | 42.156 | 28.287 | 102.768 | 1.00 | 22.76 | 6 |
| ATOM | 1075 | OG | SER | B | 336 | 41.969 | 28.552 | 101.390 | 1.00 | 26.43 | 8 |
| ATOM | 1076 | N | TYR | B | 337 | 43.118 | 31.293 | 102.075 | 1.00 | 20.56 | 7 |
| ATOM | 1077 | CA | TYR | B | 337 | 44.099 | 32.211 | 101.494 | 1.00 | 20.39 | 6 |
| ATOM | 1078 | C | TYR | B | 337 | 44.572 | 33.168 | 102.501 | 1.00 | 20.26 | 6 |
| ATOM | 1079 | O | TYR | B | 337 | 45.771 | 33.406 | 102.657 | 1.00 | 18.58 | 8 |
| ATOM | 1080 | CB | TYR | B | 337 | 43.532 | 32.993 | 100.338 | 1.00 | 20.73 | 6 |
| ATOM | 1081 | CG | TYR | B | 337 | 43.852 | 32.189 | 99.151 | 1.00 | 20.00 | 6 |
| ATOM | 1082 | CD1 | TYR | B | 337 | 45.060 | 32.300 | 98.520 | 1.00 | 19.20 | 6 |
| ATOM | 1083 | CD2 | TYR | B | 337 | 42.898 | 31.287 | 98.708 | 1.00 | 18.26 | 6 |
| ATOM | 1084 | CE1 | TYR | B | 337 | 45.333 | 31.482 | 97.449 | 1.00 | 20.11 | 6 |
| ATOM | 1085 | CE2 | TYR | B | 337 | 43.162 | 30.463 | 97.639 | 1.00 | 18.81 | 6 |
| ATOM | 1086 | CZ | TYR | B | 337 | 44.394 | 30.561 | 97.010 | 1.00 | 18.76 | 6 |
| ATOM | 1087 | OH | TYR | B | 337 | 44.690 | 29.740 | 95.950 | 1.00 | 21.89 | 8 |
| ATOM | 1088 | N | GLY | B | 338 | 43.638 | 33.720 | 103.251 | 1.00 | 20.86 | 7 |
| ATOM | 1089 | CA | GLY | B | 338 | 43.953 | 34.698 | 104.265 | 1.00 | 21.55 | 6 |
| ATOM | 1090 | C | GLY | B | 338 | 43.695 | 36.045 | 103.618 | 1.00 | 21.71 | 6 |
| ATOM | 1091 | O | GLY | B | 338 | 44.521 | 36.961 | 103.673 | 1.00 | 23.64 | 8 |
| ATOM | 1092 | N | ILE | B | 339 | 42.526 | 36.135 | 102.977 | 1.00 | 20.19 | 7 |
| ATOM | 1093 | CA | ILE | B | 339 | 42.229 | 37.339 | 102.256 | 1.00 | 17.89 | 6 |
| ATOM | 1094 | C | ILE | B | 339 | 40.826 | 37.495 | 101.770 | 1.00 | 17.77 | 6 |
| ATOM | 1095 | O | ILE | B | 339 | 39.985 | 36.578 | 101.759 | 1.00 | 17.95 | 8 |
| ATOM | 1096 | CB | ILE | B | 339 | 43.114 | 37.421 | 100.997 | 1.00 | 16.61 | 6 |
| ATOM | 1097 | CG1 | ILE | B | 339 | 42.682 | 36.303 | 100.058 | 1.00 | 17.70 | 6 |
| ATOM | 1098 | CG2 | ILE | B | 339 | 44.577 | 37.285 | 101.345 | 1.00 | 15.97 | 6 |
| ATOM | 1099 | CD1 | ILE | B | 339 | 43.095 | 36.516 | 98.623 | 1.00 | 18.76 | 6 |
| ATOM | 1100 | N | PRO | B | 340 | 40.643 | 38.716 | 101.356 | 1.00 | 18.40 | 7 |
| ATOM | 1101 | CA | PRO | B | 340 | 39.378 | 39.039 | 100.834 | 1.00 | 18.85 | 6 |
| ATOM | 1102 | C | PRO | B | 340 | 38.833 | 38.168 | 99.767 | 1.00 | 20.47 | 6 |
| ATOM | 1103 | O | PRO | B | 340 | 39.235 | 37.066 | 99.417 | 1.00 | 20.43 | 8 |
| ATOM | 1104 | CB | PRO | B | 340 | 39.484 | 40.498 | 100.459 | 1.00 | 21.51 | 6 |
| ATOM | 1105 | CG | PRO | B | 340 | 40.300 | 41.028 | 101.584 | 1.00 | 21.02 | 6 |
| ATOM | 1106 | CD | PRO | B | 340 | 41.087 | 39.867 | 102.138 | 1.00 | 20.38 | 6 |
| ATOM | 1107 | N | TYR | B | 341 | 37.855 | 38.871 | 99.290 | 1.00 | 19.86 | 7 |
| ATOM | 1108 | CA | TYR | B | 341 | 36.973 | 38.491 | 98.291 | 1.00 | 20.61 | 6 |
| ATOM | 1109 | C | TYR | B | 341 | 35.904 | 39.604 | 98.183 | 1.00 | 21.47 | 6 |
| ATOM | 1110 | O | TYR | B | 341 | 35.267 | 39.906 | 99.191 | 1.00 | 22.73 | 8 |
| ATOM | 1111 | CB | TYR | B | 341 | 36.350 | 37.149 | 98.693 | 1.00 | 19.26 | 6 |
| ATOM | 1112 | CG | TYR | B | 341 | 35.521 | 36.752 | 97.553 | 1.00 | 19.28 | 6 |
| ATOM | 1113 | CD1 | TYR | B | 341 | 36.113 | 36.701 | 96.325 | 1.00 | 20.55 | 6 |
| ATOM | 1114 | CD2 | TYR | B | 341 | 34.149 | 36.503 | 97.674 | 1.00 | 17.12 | 6 |
| ATOM | 1115 | CE1 | TYR | B | 341 | 35.364 | 36.410 | 95.214 | 1.00 | 19.33 | 6 |
| ATOM | 1116 | CE2 | TYR | B | 341 | 33.388 | 36.240 | 96.568 | 1.00 | 17.88 | 6 |
| ATOM | 1117 | CZ | TYR | B | 341 | 34.005 | 36.193 | 95.325 | 1.00 | 19.65 | 6 |
| ATOM | 1118 | OH | TYR | B | 341 | 33.276 | 35.940 | 94.192 | 1.00 | 19.58 | 8 |
| ATOM | 1119 | N | ILE | B | 342 | 35.748 | 40.219 | 96.993 | 1.00 | 20.40 | 7 |
| ATOM | 1120 | CA | ILE | B | 342 | 34.757 | 41.266 | 96.746 | 1.00 | 17.21 | 6 |
| ATOM | 1121 | C | ILE | B | 342 | 34.189 | 41.048 | 95.371 | 1.00 | 17.08 | 6 |
| ATOM | 1122 | O | ILE | B | 342 | 34.921 | 40.832 | 94.395 | 1.00 | 13.95 | 8 |
| ATOM | 1123 | CB | ILE | B | 342 | 35.361 | 42.686 | 96.796 | 1.00 | 18.65 | 6 |
| ATOM | 1124 | CG1 | ILE | B | 342 | 35.697 | 43.100 | 98.225 | 1.00 | 19.16 | 6 |
| ATOM | 1125 | CG2 | ILE | B | 342 | 34.414 | 43.692 | 96.165 | 1.00 | 16.96 | 6 |
| ATOM | 1126 | CD1 | ILE | B | 342 | 36.549 | 44.346 | 98.309 | 1.00 | 15.59 | 6 |
| ATOM | 1127 | N | GLU | B | 343 | 32.877 | 41.091 | 95.266 | 1.00 | 15.73 | 7 |
| ATOM | 1128 | CA | GLU | B | 343 | 32.177 | 40.894 | 94.003 | 1.00 | 16.49 | 6 |
| ATOM | 1129 | C | GLU | B | 343 | 32.016 | 42.246 | 93.273 | 1.00 | 15.41 | 6 |
| ATOM | 1130 | O | GLU | B | 343 | 31.543 | 43.208 | 93.881 | 1.00 | 14.98 | 8 |
| ATOM | 1131 | CB | GLU | B | 343 | 30.854 | 40.175 | 94.248 | 1.00 | 16.75 | 6 |
| ATOM | 1132 | CG | GLU | B | 343 | 31.005 | 38.776 | 94.836 | 1.00 | 21.78 | 6 |
| ATOM | 1133 | CD | GLU | B | 343 | 29.866 | 37.855 | 94.510 | 1.00 | 23.12 | 6 |
| ATOM | 1134 | OE1 | GLU | B | 343 | 28.745 | 38.347 | 94.255 | 1.00 | 26.37 | 8 |
| ATOM | 1135 | OE2 | GLU | B | 343 | 30.078 | 36.623 | 94.511 | 1.00 | 24.53 | 8 |
| ATOM | 1136 | N | THR | B | 344 | 32.387 | 42.334 | 91.973 | 1.00 | 13.00 | 7 |
| ATOM | 1137 | CA | THR | B | 344 | 32.351 | 43.614 | 91.228 | 1.00 | 11.49 | 6 |
| ATOM | 1138 | C | THR | B | 344 | 31.711 | 43.542 | 89.838 | 1.00 | 12.27 | 6 |
| ATOM | 1139 | O | THR | B | 344 | 31.422 | 42.459 | 89.336 | 1.00 | 9.62 | 8 |
| ATOM | 1140 | CB | THR | B | 344 | 33.792 | 44.112 | 90.956 | 1.00 | 10.85 | 6 |
| ATOM | 1141 | OG1 | THR | B | 344 | 34.463 | 43.173 | 90.106 | 1.00 | 6.78 | 8 |
| ATOM | 1142 | CG2 | THR | B | 344 | 34.550 | 44.273 | 92.254 | 1.00 | 8.15 | 6 |
| ATOM | 1143 | N | SER | B | 345 | 31.472 | 44.690 | 89.216 | 1.00 | 11.09 | 7 |
| ATOM | 1144 | CA | SER | B | 345 | 30.976 | 44.719 | 87.827 | 1.00 | 13.01 | 6 |
| ATOM | 1145 | C | SER | B | 345 | 31.542 | 45.927 | 87.098 | 1.00 | 14.26 | 6 |
| ATOM | 1146 | O | SER | B | 345 | 30.972 | 47.023 | 87.174 | 1.00 | 13.73 | 8 |
| ATOM | 1147 | CB | SER | B | 345 | 29.466 | 44.750 | 87.743 | 1.00 | 14.15 | 6 |
| ATOM | 1148 | OG | SER | B | 345 | 29.041 | 44.858 | 86.395 | 1.00 | 13.24 | 8 |
| ATOM | 1149 | N | ALA | B | 346 | 32.660 | 45.781 | 86.408 | 1.00 | 13.33 | 7 |
| ATOM | 1150 | CA | ALA | B | 346 | 33.220 | 46.904 | 85.669 | 1.00 | 12.18 | 6 |
| ATOM | 1151 | C | ALA | B | 346 | 32.136 | 47.551 | 84.804 | 1.00 | 13.44 | 6 |
| ATOM | 1152 | O | ALA | B | 346 | 32.363 | 48.596 | 84.196 | 1.00 | 13.49 | 8 |
| ATOM | 1153 | CB | ALA | B | 346 | 34.378 | 46.454 | 84.787 | 1.00 | 10.34 | 6 |
| ATOM | 1154 | N | LYS | B | 347 | 30.962 | 46.906 | 84.763 | 1.00 | 12.65 | 7 |
| ATOM | 1155 | CA | LYS | B | 347 | 29.857 | 47.359 | 83.912 | 1.00 | 15.70 | 6 |
| ATOM | 1156 | C | LYS | B | 347 | 29.010 | 48.385 | 84.592 | 1.00 | 15.47 | 6 |
| ATOM | 1157 | O | LYS | B | 347 | 28.598 | 49.367 | 83.964 | 1.00 | 16.65 | 8 |
| ATOM | 1158 | CB | LYS | B | 347 | 28.963 | 46.185 | 83.472 | 1.00 | 19.13 | 6 |
| ATOM | 1159 | CG | LYS | B | 347 | 27.673 | 46.627 | 82.800 | 1.00 | 20.67 | 6 |
| ATOM | 1160 | CD | LYS | B | 347 | 27.126 | 45.557 | 81.849 | 1.00 | 24.06 | 6 |
| ATOM | 1161 | CE | LYS | B | 347 | 25.916 | 46.033 | 81.054 | 1.00 | 26.11 | 6 |
| ATOM | 1162 | NZ | LYS | B | 347 | 25.201 | 44.895 | 80.406 | 1.00 | 29.99 | 7 |
| ATOM | 1163 | N | THR | B | 348 | 28.745 | 48.185 | 85.852 | 1.00 | 16.90 | 7 |
| ATOM | 1164 | CA | THR | B | 348 | 27.909 | 49.132 | 86.527 | 1.00 | 17.62 | 6 |
| ATOM | 1165 | C | THR | B | 348 | 28.724 | 49.884 | 87.548 | 1.00 | 20.92 | 6 |
| ATOM | 1166 | O | THR | B | 348 | 28.405 | 50.990 | 87.988 | 1.00 | 24.25 | 8 |
| ATOM | 1167 | CB | THR | B | 348 | 26.788 | 48.377 | 87.212 | 1.00 | 15.61 | 6 |
| ATOM | 1168 | OG1 | THR | B | 348 | 27.338 | 47.446 | 88.141 | 1.00 | 20.18 | 8 |
| ATOM | 1169 | CG2 | THR | B | 348 | 25.949 | 47.635 | 86.176 | 1.00 | 15.65 | 6 |
| ATOM | 1170 | N | ARG | B | 349 | 29.783 | 49.187 | 87.889 | 1.00 | 20.17 | 7 |
| ATOM | 1171 | CA | ARG | B | 349 | 30.729 | 49.659 | 88.856 | 1.00 | 19.87 | 6 |
| ATOM | 1172 | C | ARG | B | 349 | 30.452 | 49.101 | 90.217 | 1.00 | 19.65 | 6 |
| ATOM | 1173 | O | ARG | B | 349 | 30.739 | 49.729 | 91.238 | 1.00 | 21.24 | 8 |
| ATOM | 1174 | CB | ARG | B | 349 | 30.738 | 51.183 | 88.911 | 1.00 | 21.41 | 6 |
| ATOM | 1175 | CG | ARG | B | 349 | 29.915 | 51.862 | 87.832 | 1.00 | 23.33 | 6 |
| ATOM | 1176 | CD | ARG | B | 349 | 30.103 | 53.364 | 87.882 | 1.00 | 26.23 | 6 |
| ATOM | 1177 | NE | ARG | B | 349 | 29.333 | 54.046 | 86.849 | 1.00 | 29.02 | 7 |
| ATOM | 1178 | CZ | ARG | B | 349 | 29.856 | 54.885 | 85.964 | 1.00 | 28.12 | 6 |
| ATOM | 1179 | NE1 | ARG | B | 349 | 31.157 | 55.144 | 85.986 | 1.00 | 29.44 | 7 |
| ATOM | 1180 | NH2 | ARG | B | 349 | 29.081 | 55.461 | 85.056 | 1.00 | 26.81 | 7 |
| ATOM | 1181 | N | GLN | B | 350 | 29.900 | 47.909 | 90.237 | 1.00 | 19.31 | 7 |
| ATOM | 1182 | CA | GLN | B | 350 | 29.647 | 47.320 | 91.491 | 1.00 | 20.61 | 6 |
| ATOM | 1183 | C | GLN | B | 350 | 30.958 | 47.037 | 92.199 | 1.00 | 18.91 | 6 |
| ATOM | 1184 | O | GLN | B | 350 | 31.787 | 46.255 | 91.707 | 1.00 | 20.14 | 8 |
| ATOM | 1185 | CB | GLN | B | 350 | 28.863 | 46.030 | 91.334 | 1.00 | 22.74 | 6 |
| ATOM | 1186 | CG | GLN | B | 350 | 28.811 | 45.172 | 92.581 | 1.00 | 28.13 | 6 |
| ATOM | 1187 | CD | GLN | B | 350 | 28.112 | 43.870 | 92.279 | 1.00 | 33.79 | 6 |
| ATOM | 1188 | OE1 | GLN | B | 350 | 27.228 | 43.823 | 91.432 | 1.00 | 35.61 | 8 |
| ATOM | 1189 | NE2 | GLN | B | 350 | 28.340 | 42.703 | 92.853 | 1.00 | 36.94 | 7 |
| ATOM | 1190 | N | GLY | B | 351 | 31.147 | 47.666 | 93.347 | 1.00 | 15.77 | 7 |
| ATOM | 1191 | CA | GLY | B | 351 | 32.301 | 47.446 | 94.178 | 1.00 | 14.29 | 6 |
| ATOM | 1192 | C | GLY | B | 351 | 33.644 | 47.596 | 93.494 | 1.00 | 16.18 | 6 |
| ATOM | 1193 | O | GLY | B | 351 | 34.615 | 46.970 | 93.917 | 1.00 | 16.35 | 8 |
| ATOM | 1194 | N | VAL | B | 352 | 33.734 | 48.431 | 92.449 | 1.00 | 15.01 | 7 |
| ATOM | 1195 | CA | VAL | B | 352 | 35.038 | 48.627 | 91.788 | 1.00 | 16.69 | 6 |
| ATOM | 1196 | C | VAL | B | 352 | 36.066 | 49.312 | 92.705 | 1.00 | 18.61 | 6 |
| ATOM | 1197 | O | VAL | B | 352 | 37.268 | 49.011 | 92.666 | 1.00 | 18.11 | 8 |
| ATOM | 1198 | CB | VAL | B | 352 | 34.924 | 49.433 | 90.453 | 1.00 | 16.08 | 6 |
| ATOM | 1199 | CG1 | VAL | B | 352 | 36.297 | 49.789 | 89.913 | 1.00 | 14.40 | 6 |
| ATOM | 1200 | CG2 | VAL | B | 352 | 34.142 | 48.625 | 89.422 | 1.00 | 13.93 | 6 |
| ATOM | 1201 | N | GLU | B | 353 | 35.584 | 50.216 | 93.522 | 1.00 | 20.53 | 7 |
| ATOM | 1202 | CA | GLU | B | 353 | 36.397 | 50.913 | 94.475 | 1.00 | 22.53 | 6 |
| ATOM | 1203 | C | GLU | B | 353 | 36.769 | 50.019 | 95.611 | 1.00 | 21.83 | 6 |
| ATOM | 1204 | O | GLU | B | 353 | 37.951 | 49.812 | 95.920 | 1.00 | 22.92 | 8 |
| ATOM | 1205 | CB | GLU | B | 353 | 35.593 | 52.033 | 95.125 | 1.00 | 24.74 | 6 |
| ATOM | 1206 | CG | GLU | B | 353 | 35.794 | 53.394 | 94.551 | 1.00 | 30.79 | 6 |
| ATOM | 1207 | CD | GLU | B | 353 | 35.144 | 54.396 | 95.450 | 1.00 | 33.69 | 6 |
| ATOM | 1208 | OE1 | GLU | B | 353 | 35.511 | 54.464 | 96.634 | 1.00 | 37.05 | 8 |
| ATOM | 1209 | OE2 | GLU | B | 353 | 34.258 | 55.121 | 94.959 | 1.00 | 35.15 | 8 |
| ATOM | 1210 | N | ASP | B | 354 | 35.752 | 49.509 | 96.230 | 1.00 | 21.41 | 7 |
| ATOM | 1211 | CA | ASP | B | 354 | 36.122 | 48.698 | 97.329 | 1.00 | 21.01 | 6 |
| ATOM | 1212 | C | ASP | B | 354 | 37.239 | 47.781 | 96.949 | 1.00 | 20.27 | 6 |
| ATOM | 1213 | O | ASP | B | 354 | 38.269 | 47.720 | 97.623 | 1.00 | 17.50 | 8 |
| ATOM | 1214 | CB | ASP | B | 354 | 34.970 | 47.914 | 97.845 | 1.00 | 24.36 | 6 |
| ATOM | 1215 | CG | ASP | B | 354 | 34.995 | 48.038 | 99.330 | 1.00 | 30.77 | 6 |
| ATOM | 1216 | OD1 | ASP | B | 354 | 35.665 | 47.203 | 99.993 | 1.00 | 33.05 | 8 |
| ATOM | 1217 | OD2 | ASP | B | 354 | 34.336 | 48.956 | 99.861 | 1.00 | 31.44 | 8 |
| ATOM | 1218 | N | ALA | B | 355 | 37.039 | 47.068 | 95.866 | 1.00 | 17.49 | 7 |
| ATOM | 1219 | CA | ALA | B | 355 | 38.023 | 46.136 | 95.458 | 1.00 | 15.18 | 6 |
| ATOM | 1220 | C | ALA | B | 355 | 39.415 | 46.748 | 95.446 | 1.00 | 16.54 | 6 |
| ATOM | 1221 | O | ALA | B | 355 | 40.322 | 46.156 | 96.032 | 1.00 | 18.35 | 8 |
| ATOM | 1222 | CB | ALA | B | 355 | 37.698 | 45.567 | 94.085 | 1.00 | 15.15 | 6 |
| ATOM | 1223 | N | PHE | B | 356 | 39.616 | 47.904 | 94.803 | 1.00 | 15.85 | 7 |
| ATOM | 1224 | CA | PHE | B | 356 | 40.940 | 48.508 | 94.696 | 1.00 | 16.22 | 6 |
| ATOM | 1225 | C | PHE | B | 356 | 41.396 | 49.169 | 95.995 | 1.00 | 17.66 | 6 |
| ATOM | 1226 | O | PHE | B | 356 | 42.571 | 49.084 | 96.362 | 1.00 | 14.98 | 8 |
| ATOM | 1227 | CB | PHE | B | 356 | 40.961 | 49.522 | 93.559 | 1.00 | 15.23 | 6 |
| ATOM | 1228 | CG | PHE | B | 356 | 41.158 | 48.905 | 92.202 | 1.00 | 14.51 | 6 |
| ATOM | 1229 | CD1 | PHE | B | 356 | 42.427 | 48.602 | 91.713 | 1.00 | 14.99 | 6 |
| ATOM | 1230 | CD2 | PHE | B | 356 | 40.054 | 48.645 | 91.402 | 1.00 | 12.98 | 6 |
| ATOM | 1231 | CE1 | PHE | B | 356 | 42.591 | 48.051 | 90.446 | 1.00 | 14.46 | 6 |
| ATOM | 1232 | CE2 | PHE | B | 356 | 40.209 | 48.093 | 90.132 | 1.00 | 15.29 | 6 |
| ATOM | 1233 | CZ | PHE | B | 356 | 41.483 | 47.796 | 89.654 | 1.00 | 12.95 | 6 |
| ATOM | 1234 | N | TYR | B | 357 | 40.467 | 49.834 | 96.680 | 1.00 | 19.54 | 7 |
| ATOM | 1235 | CA | TYR | B | 357 | 40.770 | 50.522 | 97.932 | 1.00 | 20.37 | 6 |
| ATOM | 1236 | C | TYR | B | 357 | 41.050 | 49.544 | 99.063 | 1.00 | 19.02 | 6 |
| ATOM | 1237 | O | TYR | B | 357 | 41.887 | 49.811 | 99.927 | 1.00 | 20.01 | 8 |
| ATOM | 1238 | CB | TYR | B | 357 | 39.640 | 51.486 | 98.312 | 1.00 | 21.80 | 6 |
| ATOM | 1239 | CG | TYR | B | 357 | 39.631 | 52.753 | 97.483 | 1.00 | 23.79 | 6 |
| ATOM | 1240 | CD1 | TYR | B | 357 | 40.662 | 53.027 | 96.582 | 1.00 | 25.01 | 6 |
| ATOM | 1241 | CD2 | TYR | B | 357 | 38.602 | 53.683 | 97.607 | 1.00 | 26.46 | 6 |
| ATOM | 1242 | CE1 | TYR | B | 357 | 40.673 | 54.191 | 95.831 | 1.00 | 28.21 | 6 |
| ATOM | 1243 | CE2 | TYR | B | 357 | 38.604 | 54.857 | 96.859 | 1.00 | 30.15 | 6 |
| ATOM | 1244 | CZ | TYR | B | 357 | 39.644 | 55.103 | 95.974 | 1.00 | 30.94 | 6 |
| ATOM | 1245 | OH | TYR | B | 357 | 39.666 | 56.266 | 95.237 | 1.00 | 34.33 | 8 |
| ATOM | 1246 | N | THR | B | 358 | 40.354 | 48.409 | 99.044 | 1.00 | 17.62 | 7 |
| ATOM | 1247 | CA | THR | B | 358 | 40.553 | 47.379 | 100.055 | 1.00 | 15.39 | 6 |
| ATOM | 1248 | C | THR | B | 358 | 41.936 | 46.757 | 99.879 | 1.00 | 13.79 | 6 |
| ATOM | 1249 | O | THR | B | 358 | 42.572 | 46.345 | 100.851 | 1.00 | 16.35 | 8 |
| ATOM | 1250 | CB | THR | B | 358 | 39.474 | 46.290 | 99.968 | 1.00 | 15.92 | 6 |
| ATOM | 1251 | OG1 | THR | B | 358 | 38.185 | 46.879 | 100.189 | 1.00 | 16.52 | 8 |
| ATOM | 1252 | CG2 | THR | B | 358 | 39.714 | 45.212 | 101.009 | 1.00 | 16.77 | 6 |
| ATOM | 1253 | N | LEU | B | 359 | 42.410 | 46.708 | 98.638 | 1.00 | 10.71 | 7 |
| ATOM | 1254 | CA | LEU | B | 359 | 43.730 | 46.158 | 98.364 | 1.00 | 10.63 | 6 |
| ATOM | 1255 | C | LEU | B | 359 | 44.790 | 47.134 | 98.867 | 1.00 | 10.52 | 6 |
| ATOM | 1256 | O | LEU | B | 359 | 45.825 | 46.722 | 99.386 | 1.00 | 11.97 | 8 |
| ATOM | 1257 | CB | LEU | B | 359 | 43.911 | 45.900 | 96.870 | 1.00 | 6.43 | 6 |
| ATOM | 1258 | CG | LEU | B | 359 | 45.262 | 45.313 | 96.457 | 1.00 | 4.47 | 6 |
| ATOM | 1259 | CD1 | LEU | B | 359 | 45.542 | 44.017 | 97.214 | 1.00 | 7.62 | 6 |
| ATOM | 1260 | CD2 | LEU | B | 359 | 45.258 | 45.074 | 94.957 | 1.00 | 6.43 | 6 |
| ATOM | 1261 | N | VAL | B | 360 | 44.517 | 48.425 | 98.706 | 1.00 | 12.43 | 7 |
| ATOM | 1262 | CA | VAL | B | 360 | 45.418 | 49.486 | 99.160 | 1.00 | 12.53 | 6 |
| ATOM | 1263 | C | VAL | B | 360 | 45.569 | 49.435 | 100.682 | 1.00 | 12.35 | 6 |
| ATOM | 1264 | O | VAL | B | 360 | 46.675 | 49.554 | 101.209 | 1.00 | 11.56 | 8 |
| ATOM | 1265 | CB | VAL | B | 360 | 44.871 | 50.876 | 98.763 | 1.00 | 12.07 | 6 |
| ATOM | 1266 | CG1 | VAL | B | 360 | 45.701 | 51.991 | 99.400 | 1.00 | 11.14 | 6 |
| ATOM | 1267 | CG2 | VAL | B | 360 | 44.864 | 51.015 | 97.254 | 1.00 | 14.35 | 6 |
| ATOM | 1268 | N | ARG | B | 361 | 44.447 | 49.274 | 101.376 | 1.00 | 11.63 | 7 |
| ATOM | 1269 | CA | ARG | B | 361 | 44.439 | 49.198 | 102.834 | 1.00 | 15.50 | 6 |
| ATOM | 1270 | C | ARG | B | 361 | 45.174 | 47.947 | 103.291 | 1.00 | 15.39 | 6 |
| ATOM | 1271 | O | ARG | B | 361 | 45.841 | 47.953 | 104.319 | 1.00 | 17.95 | 8 |
| ATOM | 1272 | CB | ARG | B | 361 | 43.003 | 49.171 | 103.353 | 1.00 | 16.24 | 6 |
| ATOM | 1273 | CG | ARG | B | 361 | 42.224 | 50.420 | 103.015 | 1.00 | 21.26 | 6 |
| ATOM | 1274 | CD | ARG | B | 361 | 40.801 | 50.342 | 103.518 | 1.00 | 25.92 | 6 |
| ATOM | 1275 | NE | ARG | B | 361 | 40.053 | 51.545 | 103.173 | 1.00 | 26.30 | 7 |
| ATOM | 1276 | CZ | ARG | B | 361 | 39.014 | 51.572 | 102.346 | 1.00 | 25.63 | 6 |
| ATOM | 1277 | NH1 | ARG | B | 361 | 38.586 | 50.455 | 101.775 | 1.00 | 25.03 | 7 |
| ATOM | 1278 | NH2 | ARG | B | 361 | 38.410 | 52.722 | 102.086 | 1.00 | 26.64 | 7 |
| ATOM | 1279 | N | GLU | B | 362 | 45.030 | 46.881 | 102.506 | 1.00 | 18.13 | 7 |
| ATOM | 1280 | CA | GLU | B | 362 | 45.697 | 45.614 | 102.777 | 1.00 | 15.00 | 6 |
| ATOM | 1281 | C | GLU | B | 362 | 47.213 | 45.767 | 102.722 | 1.00 | 14.50 | 6 |
| ATOM | 1282 | O | GLU | B | 362 | 47.926 | 45.285 | 103.586 | 1.00 | 14.64 | 8 |
| ATOM | 1283 | CB | GLU | B | 362 | 45.232 | 44.541 | 101.791 | 1.00 | 16.54 | 6 |
| ATOM | 1284 | CG | GLU | B | 362 | 45.699 | 43.136 | 102.136 | 1.00 | 24.18 | 6 |
| ATOM | 1285 | CD | GLU | B | 362 | 44.656 | 42.348 | 102.906 | 1.00 | 24.52 | 6 |
| ATOM | 1286 | OE1 | GLU | B | 362 | 43.634 | 42.946 | 103.304 | 1.00 | 29.72 | 8 |
| ATOM | 1287 | OE2 | GLU | B | 362 | 44.861 | 41.134 | 103.111 | 1.00 | 29.14 | 8 |
| ATOM | 1288 | N | ILE | B | 363 | 47.670 | 46.490 | 101.696 | 1.00 | 13.62 | 7 |
| ATOM | 1289 | CA | ILE | B | 363 | 49.098 | 46.752 | 101.513 | 1.00 | 14.51 | 6 |
| ATOM | 1290 | C | ILE | B | 363 | 49.612 | 47.607 | 102.660 | 1.00 | 18.09 | 6 |
| ATOM | 1291 | O | ILE | B | 363 | 50.695 | 47.362 | 103.188 | 1.00 | 17.21 | 8 |
| ATOM | 1292 | CB | ILE | B | 363 | 49.381 | 47.499 | 100.189 | 1.00 | 13.80 | 6 |
| ATOM | 1293 | CG1 | ILE | B | 363 | 48.919 | 46.663 | 98.989 | 1.00 | 11.48 | 6 |
| ATOM | 1294 | CG2 | ILE | B | 363 | 50.871 | 47.815 | 100.068 | 1.00 | 12.40 | 6 |
| ATOM | 1295 | CD1 | ILE | B | 363 | 49.046 | 47.380 | 97.653 | 1.00 | 10.17 | 6 |
| ATOM | 1296 | N | ARG | B | 364 | 48.821 | 48.609 | 103.038 | 1.00 | 20.25 | 7 |
| ATOM | 1297 | CA | ARG | B | 364 | 49.170 | 49.518 | 104.127 | 1.00 | 24.82 | 6 |
| ATOM | 1298 | C | ARG | B | 364 | 49.318 | 48.752 | 105.446 | 1.00 | 25.29 | 6 |
| ATOM | 1299 | O | ARG | B | 364 | 50.225 | 49.020 | 106.241 | 1.00 | 27.02 | 8 |
| ATOM | 1300 | CB | ARG | B | 364 | 48.100 | 50.613 | 104.243 | 1.00 | 27.21 | 6 |
| ATOM | 1301 | CG | ARG | B | 364 | 48.426 | 51.754 | 105.196 | 1.00 | 30.99 | 6 |
| ATOM | 1302 | CD | ARG | B | 364 | 47.361 | 52.841 | 105.113 | 1.00 | 35.30 | 6 |
| ATOM | 1303 | NE | ARG | B | 364 | 46.017 | 52.323 | 105.384 | 1.00 | 39.11 | 7 |
| ATOM | 1304 | CZ | ARG | B | 364 | 44.891 | 52.859 | 104.916 | 1.00 | 39.38 | 6 |
| ATOM | 1305 | NH1 | ARG | B | 364 | 44.925 | 53.940 | 104.144 | 1.00 | 41.49 | 7 |
| ATOM | 1306 | NH2 | ARG | B | 364 | 43.725 | 52.313 | 105.223 | 1.00 | 41.33 | 7 |
| ATOM | 1307 | N | GLN | B | 365 | 48.435 | 47.782 | 105.656 | 1.00 | 25.79 | 7 |
| ATOM | 1308 | CA | GLN | B | 365 | 48.424 | 46.986 | 106.894 | 1.00 | 27.70 | 6 |
| ATOM | 1309 | C | GLN | B | 365 | 49.620 | 46.087 | 107.025 | 1.00 | 29.58 | 6 |
| ATOM | 1310 | O | GLN | B | 365 | 50.130 | 45.778 | 108.114 | 1.00 | 31.20 | 8 |
| ATOM | 1311 | CB | GLN | B | 365 | 47.181 | 46.095 | 106.944 | 1.00 | 29.53 | 6 |
| ATOM | 1312 | CG | GLN | B | 365 | 45.868 | 46.852 | 107.089 | 1.00 | 31.95 | 6 |
| ATOM | 1313 | CD | GLN | B | 365 | 44.645 | 46.031 | 106.720 | 1.00 | 35.63 | 6 |
| ATOM | 1314 | OE1 | GLN | B | 365 | 44.752 | 44.850 | 106.371 | 1.00 | 32.96 | 8 |
| ATOM | 1315 | NE2 | GLN | B | 365 | 43.382 | 46.444 | 106.724 | 1.00 | 35.21 | 7 |
| ATOM | 1316 | N | HIS | B | 366 | 50.021 | 45.688 | 105.838 | 1.00 | 30.22 | 7 |
| ATOM | 1317 | CA | HIS | B | 366 | 51.123 | 44.795 | 105.582 | 1.00 | 30.69 | 6 |
| ATOM | 1318 | C | HIS | B | 366 | 52.475 | 45.543 | 105.678 | 1.00 | 31.24 | 6 |
| ATOM | 1319 | O | HIS | B | 366 | 53.422 | 45.038 | 106.284 | 1.00 | 31.93 | 8 |
| ATOM | 1320 | CB | HIS | B | 366 | 50.829 | 44.130 | 104.254 | 1.00 | 31.85 | 6 |
| ATOM | 1321 | CG | HIS | B | 366 | 51.892 | 43.130 | 103.835 | 1.00 | 35.22 | 6 |
| ATOM | 1322 | ND1 | HIS | B | 366 | 52.789 | 43.401 | 102.823 | 1.00 | 35.30 | 7 |
| ATOM | 1323 | CD2 | HIS | B | 366 | 52.203 | 41.893 | 104.291 | 1.00 | 34.70 | 6 |
| ATOM | 1324 | CE1 | HIS | B | 366 | 53.606 | 42.373 | 102.673 | 1.00 | 35.81 | 6 |
| ATOM | 1325 | NE2 | HIS | B | 366 | 53.272 | 41.446 | 103.552 | 1.00 | 36.63 | 7 |
| ATOM | 1326 | PG | GNP | $ | 1380 | 42.807 | 39.954 | 78.851 | 1.00 | 8.68 | 15 |
| ATOM | 1327 | O1G | GNP | $ | 1380 | 43.395 | 39.015 | 80.005 | 1.00 | 8.34 | 8 |
| ATOM | 1328 | O2G | GNP | $ | 1380 | 42.997 | 39.263 | 77.587 | 1.00 | 11.57 | 8 |
| ATOM | 1329 | O3G | GNP | $ | 1380 | 43.676 | 41.101 | 78.946 | 1.00 | 13.99 | 8 |
| ATOM | 1330 | N3B | GNP | $ | 1380 | 41.222 | 40.232 | 79.146 | 1.00 | 7.92 | 7 |
| ATOM | 1331 | PB | GNP | $ | 1380 | 40.622 | 41.012 | 80.468 | 1.00 | 8.51 | 15 |
| ATOM | 1332 | O1B | GNP | $ | 1380 | 40.631 | 40.224 | 81.725 | 1.00 | 9.22 | 8 |
| ATOM | 1333 | O2B | GNP | $ | 1380 | 41.154 | 42.379 | 80.644 | 1.00 | 10.55 | 8 |
| ATOM | 1334 | O3A | GNP | $ | 1380 | 39.082 | 41.193 | 80.101 | 1.00 | 10.51 | 8 |
| ATOM | 1335 | PA | GNP | $ | 1380 | 38.413 | 42.583 | 79.491 | 1.00 | 9.87 | 15 |
| ATOM | 1336 | O1A | GNP | $ | 1380 | 38.420 | 43.677 | 80.506 | 1.00 | 7.44 | 8 |
| ATOM | 1337 | O2A | GNP | $ | 1380 | 38.965 | 42.868 | 78.142 | 1.00 | 13.51 | 8 |
| ATOM | 1338 | O5* | GNP | $ | 1380 | 36.910 | 42.062 | 79.346 | 1.00 | 8.97 | 8 |
| ATOM | 1339 | C5* | GNP | $ | 1380 | 36.520 | 41.047 | 78.395 | 1.00 | 10.25 | 6 |
| ATOM | 1340 | C4* | GNP | $ | 1380 | 35.053 | 41.226 | 77.960 | 1.00 | 11.24 | 6 |
| ATOM | 1341 | O4* | GNP | $ | 1380 | 34.180 | 41.173 | 79.119 | 1.00 | 11.67 | 8 |
| ATOM | 1342 | C3* | GNP | $ | 1380 | 34.841 | 42.619 | 77.313 | 1.00 | 11.16 | 6 |
| ATOM | 1343 | O3* | GNP | $ | 1380 | 33.887 | 42.627 | 76.221 | 1.00 | 19.79 | 8 |
| ATOM | 1344 | C2* | GNP | $ | 1380 | 34.324 | 43.431 | 78.498 | 1.00 | 11.63 | 6 |
| ATOM | 1345 | O2* | GNP | $ | 1380 | 33.508 | 44.570 | 78.167 | 1.00 | 8.36 | 8 |
| ATOM | 1346 | C1* | GNP | $ | 1380 | 33.466 | 42.404 | 79.238 | 1.00 | 8.70 | 6 |
| ATOM | 1347 | N9 | GNP | $ | 1380 | 33.326 | 42.740 | 80.671 | 1.00 | 8.66 | 7 |
| ATOM | 1348 | C8 | GNP | $ | 1380 | 34.314 | 43.075 | 81.566 | 1.00 | 6.11 | 6 |
| ATOM | 1349 | N7 | GNP | $ | 1380 | 33.899 | 43.320 | 82.743 | 1.00 | 8.97 | 7 |
| ATOM | 1350 | C5 | GNP | $ | 1380 | 32.538 | 43.148 | 82.661 | 1.00 | 10.36 | 6 |
| ATOM | 1351 | C6 | GNP | $ | 1380 | 31.550 | 43.287 | 83.661 | 1.00 | 12.02 | 6 |
| ATOM | 1352 | O6 | GNP | $ | 1380 | 31.711 | 43.601 | 84.850 | 1.00 | 14.72 | 8 |
| ATOM | 1353 | N1 | GNP | $ | 1380 | 30.270 | 43.018 | 83.152 | 1.00 | 9.31 | 7 |
| ATOM | 1354 | C2 | GNP | $ | 1380 | 29.979 | 42.659 | 81.852 | 1.00 | 11.23 | 6 |
| ATOM | 1355 | N2 | GNP | $ | 1380 | 28.694 | 42.440 | 81.564 | 1.00 | 8.24 | 7 |
| ATOM | 1356 | N3 | GNP | $ | 1380 | 30.923 | 42.531 | 80.915 | 1.00 | 9.36 | 7 |
| ATOM | 1357 | C4 | GNP | $ | 1380 | 32.170 | 42.789 | 81.391 | 1.00 | 9.70 | 6 |
| ATOM | 1358 | MG | MG | $ | 1373 | 41.839 | 43.269 | 77.994 | 1.00 | 34.76 | 12 |
| END | | | | | | | | | | | |

1. A crystal comprising Ras and PI3K.

2. A crystal according to claim 1, wherein the Ras and PI3K are present in the form of a complex.

3. A crystal according to claim 1 or claim 2, comprising a further molecule capable of interacting with Ras, PI3K and/or a Ras/PI3K complex.

4. A crystal according to claim 1, wherein Ras and/or PI3K is a Ras and/or PI3K variant, which variant comprises an amino acid insertion, deletion or substitution.

5. A crystal according to claim 1, wherein Ras is human Ras.

6. A crystal according to claim 1, wherein Ras is H-Ras

7. A crystal according to claim 1, wherein Ras is the Ras effector domain.

8. A crystal according to claim 1, wherein PI3K is PI3Kγ.

9. A crystal according to claim 8, wherein the PI3K is human PI3K.

10. A crystal according to claim 1, wherein the PI3K is the catalytic subunit of PI3K.

11. A crystal according to any of claim 1, wherein the PI3K comprises the mutation V223K.

12. A crystal having the structural coordinates shown in table 1.

13. A model for at least part of the Ras effector domain and/or at least part of the PI3K catalytic subunit, made using a crystal according to claim 1.

14. A method of screening for or designing a ligand capable of modulating the interaction between Ras and PI3K, comprising the use of a model according to claim 13.

15. A method of screening for or designing a ligand capable of modulating the activation of PI3K by Ras, comprising the use of a model according to claim 13.

16. A computer readable medium having stored thereon: the structure of a crystal according to claim 1, or a model for at least part of the Ras effector domain and/or at least part of the PI3K catalytic subunit according to claim 13.

17. A ligand identified by the method according to claim 14 or 15.

18. A ligand according to claim 17, which interacts with any one or more residues present in the β-2 strand of Ras or the Rβ2 strand of PI3K.

19. The use of a ligand according to claim 17, in the manufacture of a medicament to treat and/or prevent a disease in a mammalian patient.

20. A pharmaceutical composition comprising a ligand according to claim 17 and optionally a pharmaceutically acceptable carrier, diluent, excipient or adjuvant or any combination thereof.

21. A method of treating and/or preventing a disease comprising administering a ligand according to claim 17 a mammalian patient.

22. Method of claim 21 further comprising administering a pharmaceutical composition according to claim 20.

23. A method of treating and/or preventing a disease comprising administering a pharmaceutical composition according to claim 20.
